SEQUENCE LISTING (1176 sequences)

SEQ ID NO: 1-23 - skipped (23 entries)

SEQ ID NO: 24 - protein, 361 aa, unidentified
  REGION 1..361: note = Description of Unknown: proTGFBETA1 sequence
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 25 - protein, 361 aa, unidentified
  REGION 1..361: note = Description of Unknown: proTGFBETA1 C4S sequence
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - protein, 360 aa, unidentified
  REGION 1..360: note = Description of Unknown: proTGFBETA1 D2G sequence
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 27 - protein, 360 aa, unidentified
  REGION 1..360: note = Description of Unknown: proTGFBETA1 C4S D2G sequence
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 28 - protein, 395 aa, unidentified
  REGION 1..395: note = Description of Unknown: proTGFBETA2 sequence
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 29 - protein, 395 aa, unidentified
  REGION 1..395: note = Description of Unknown: proTGFBETA2 C5S sequence
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 30 - protein, 394 aa, unidentified
  REGION 1..394: note = Description of Unknown: proTGFBETA2 C5S D2G sequence
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 31 - protein, 394 aa, unidentified
  REGION 1..394: note = Description of Unknown: proTGFBETA2 D2G sequence
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 32 - protein, 392 aa, unidentified
  REGION 1..392: note = Description of Unknown: proTGFBETA3 sequence
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 33 - protein, 392 aa, unidentified
  REGION 1..392: note = Description of Unknown: proTGFBETA3 C7S sequence
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 34 - protein, 391 aa, unidentified
  REGION 1..391: note = Description of Unknown: proTGFBETA3 C7S D2G sequence
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 35 - protein, 391 aa, unidentified
  REGION 1..391: note = Description of Unknown: proTGFBETA3 D2G sequence
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 36 - protein, 361 aa, Mus sp.
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 37 - protein, 361 aa, Macaca fascicularis
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 38 - protein, 249 aa, Mus sp.
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 39 - protein, 249 aa, Macaca fascicularis
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRR

SEQ ID NO: 40 - protein, 360 aa, Mus sp.
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHGALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 41 - protein, 361 aa, Mus sp.
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 42 - protein, 361 aa, Macaca fascicularis
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 43 - protein, 360 aa, Macaca fascicularis
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 44 - protein, 1260 aa, Macaca fascicularis
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRAGALSTGALPPLAPEGDSVASKHAIYAVQVIADPPGPGEGPPAQHAAFLVPLGPGQISAEVQAPPPVVNVRVHHPPEASVQVHRIESSNAEGAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPADGTAAFKEICPAGKGYHILTSHQTLTIQGESDFSLFLHPDGPPKPQQLPESPSQAPPPEDTEEERGVTTDSPVSEERSVQQSHPTATTSPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCVDVDECEAGDVCDNGICTNTPGSFQCQCLSGYHLSRDRSHCEDIDECDFPAACIGGDCINTNGSYRCLCPQGHRLVGGRKCQDIDECTQDPGLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGAEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPQAGPALTFDDCCCRQGRGWGAQCRPCPPRGAGSQCPTSQSESNSFWDTSPLLLGKPRRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 45 - protein, 1228 aa, Mus sp.
GPAGERGTGGGGALARERFKVVFAPVICKRTCLKGQCRDSCQQGSNMTLIGENGHSTDTLTGSAFRVVVCPLPCMNGGQCSSRNQCLCPPDFTGRFCQVPAAGTGAGTGSSGPGLARTGAMSTGPLPPLAPEGESVASKHAIYAVQVIADPPGPGEGPPAQHAAFLVPLGPGQISAEVQAPPPVVNVRVHHPPEASVQVHRIEGPNAEGPASSQHLLPHPKPPHPRPPTQKPLGRCFQDTLPKQPCGSNPLPGLTKQEDCCGSIGTAWGQSKCHKCPQLQYTGVQKPVPVRGEVGADCPQGYKRLNSTHCQDINECAMPGNVCHGDCLNNPGSYRCVCPPGHSLGPLAAQCIADKPEEKSLCFRLVSTEHQCQHPLTTRLTRQLCCCSVGKAWGARCQRCPADGTAAFKEICPGKGYHILTSHQTLTIQGESDFSLFLHPDGPPKPQQLPESPSRAPPLEDTEEERGVTMDPPVSEERSVQQSHPTTTTSPPRPYPELISRPSPPTFHRFLPDLPPSRSAVEIAPTQVTETDECRLNQNICGHGQCVPGPSDYSCHCNAGYRSHPQHRYCVDVNECEAEPCGPGKGICMNTGGSYNCHCNRGYRLHVGAGGRSCVDLNECAKPHLCGDGGFCINFPGHYKCNCYPGYRLKASRPPICEDIDECRDPSTCPDGKCENKPGSFKCIACQPGYRSQGGGACRDVNECSEGTPCSPGWCENLPGSYRCTCAQYEPAQDGLSCIDVDECEAGKVCQDGICTNTPGSFQCQCLSGYHLSRDRSRCEDIDECDFPAACIGGDCINTNGSYRCLCPLGHRLVGGRKCKKDIDECSQDPGLCLPHACENLQGSYVCVCDEGFTLTQDQHGCEEVEQPHHKKECYLNFDDTVFCDSVLATNVTQQECCCSLGAGWGDHCEIYPCPVYSSAEFHSLVPDGKRLHSGQQHCELCIPAHRDIDECILFGAEICKEGKCVNTQPGYECYCKQGFYYDGNLLECVDVDECLDESNCRNGVCENTRGGYRCACTPPAEYSPAQAQCLIPERWSTPQRDVKCAGASEERTACVWGPWAGPALTFDDCCCRQPRLGTQCRPCPPRGTGSQCPTSQSESNSFWDTSPLLLGKSPRDEDSSEEDSDECRCVSGRCVPRPGGAVCECPGGFQLDASRARCVDIDECRELNQRGLLCKSERCVNTSGSFRCVCKAGFTRSRPHGPACLSAAADDAAIAHTSVIDHRGYFH

SEQ ID NO: 46 - protein, 1373 aa, Macaca fascicularis
NHTGRIKVVFTPSICKVTCTKGSCQNSCEKGNTTTLISENGHAADTLTATNFRVVLCHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYKFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCTNGDCSNLEGSYMCSCHKGYTRTPDHKHCKDIDECQQGNLCVNGQCKNTEGSFRCTCGQGYQLSAAKDQCEDIDECQHHHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVEQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPMKDSDDYAQLCNIPVTGRRQPYGRDALVDFSEQYAPEADPYFIQDRFLNSFEELQAEECGILNGCENGRCVRVQEGYTCDCFDGYHLDTAKMTCVDVNECDELNNRMSLCKNAKCINTEGSYKCLCLPGYVPSDKPNYCTPLNTALNLEKDSDLE

SEQ ID NO: 47 - protein, 1374 aa, Mus sp.
NHTGRIKVVFTPSICKVTCTKGNCQNSCQKGNTTTLISENGHAADTLTATNFRVVICHLPCMNGGQCSSRDKCQCPPNFTGKLCQIPVLGASMPKLYQHAQQQGKALGSHVIHSTHTLPLTMTSQQGVKVKFPPNIVNIHVKHPPEASVQIHQVSRIDSPGGQKVKEAQPGQSQVSYQGLPVQKTQTVHSTYSHQQLIPHVYPVAAKTQLGRCFQETIGSQCGKALPGLSKQEDCCGTVGTSWGFNKCQKCPKKQSYHGYTQMMECLQGYKRVNNTFCQDINECQLQGVCPNGECLNTMGSYRCSCKMGFGPDPTFSSCVPDPPVISEEKGPCYRLVSPGRHCMHPLSVHLTKQICCCSVGKAWGPHCEKCPLPGTAAFKEICPGGMGYTVSGVHRRRPIHQHIGKEAVYVKPKNTQPVAKSTHPPPLPAKEEPVEALTSSWEHGPRGAEPEVVTAPPEKEIPSLDQEKTRLEPGQPQLSPGVSTIHLHPQFPVVVEKTSPPVPVEVAPEASTSSASQVIAPTQVTEINECTVNPDICGAGHCINLPVRYTCICYEGYKFSEQLRKCVDIDECAQVRHLCSQGRCENTEGSFLCVCPAGFMASEEGTNCIDVDECLRPDMCRDGRCINTAGAFRCEYCDSGYRMSRRGYCEDIDECLKPSTCPEEQCVNTPGSYQCVPCTEGFRGWNGQCLDVDECLQPKVCTNGSCTNLEGSYMCSCHRGYSPTPDHRHCQDIDECQQGNLCMNGQCRNTDGSFRCTCGQGYQLSAAKDQCEDIDECEHHHLCSHGQCRNTEGSFQCVCNQGYRASVLGDHCEDINECLEDSSVCQGGDCINTAGSYDCTCPDGFQLNDNKGCQDINECAQPGLCGSHGECLNTQGSFHCVCEQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRVTEDSGVDRQPREEKKECYYNLNDASLCDNVLAPNVTKQECCCTSGAGWGDNCEIFPCPVQGTAEFTEMCPRGKGLVPAGESSYDTGGENYKDADECLLFGEEICKNGYCLNTQPGYECYCKQGTYYDPVKLQCFDMDECQDPNSCIDGQCVNTEGSYNCFCTHPMVLDASEKRCVQPTESNEQIEETDVYQDLCWEHLSEEYVCSRPLVGKQTTYTECCCLYGEAWGMQCALCPMKDSDDYAQLCNIPVTGRRRPYGRDALVDFSEQYGPETDPYFIQDRFLNSFEELQAEECGILNGCENGRCVRVQEGYTCDCFDGYHLDMAKMTCVDVNECSELNNRMSLCKNAKCINTEGSYKCLCLPGYIPSDKPNYCTPLNSALNLDKESDLE

SEQ ID NO: 48 - protein, 646 aa, Mus sp.
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVNLILLLSFTLVSAIVLTTLATICFLRRQKLSQQYKA

SEQ ID NO: 49 - protein, 611 aa, Mus sp.
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVN

SEQ ID NO: 50 - protein, 1375 aa, unidentified
  REGION 1..1375: note = Description of Unknown: LTBP1S sequence
NHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGVGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 51 - protein, 1260 aa, unidentified
  REGION 1..1260: note = Description of Unknown: LTBP3 sequence
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRTGALSTGALPPLAPEGDSVASKHAIYAVQVIADPPGPGEGPPAQHAAFLVPLGPGQISAEVQAPPPVVNVRVHHPPEASVQVHRIESSNAESAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPTDGTAAFKEICPAGKGYHILTSHQTLTIQGESDFSLFLHPDGPPKPQQLPESPSQAPPPEDTEEERGVTTDSPVSEERSVQQSHPTATTTPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCLDVDECEAGDVCDNGICSNTPGSFQCQCLSGYHLSRDRSHCEDIDECDFPAACIGGDCINTNGSYRCLCPQGHRLVGGRKCQDIDECSQDPSLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGSEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPLAGPALTFDDCCCRQGRGWGAQCRPCPPRGAGSHCPTSQSESNSFWDTSPLLLGKPPRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 52 - protein, 645 aa, unidentified
  REGION 1..645: note = Description of Unknown: GARP sequence
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 53 - protein, 610 aa, unidentified
  REGION 1..610: note = Description of Unknown: sGARP sequence
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNIN

SEQ ID NO: 54 - protein, 5 aa, synthetic construct
  REGION 1..5: note = Description of Artificial Sequence: Synthetic peptide
CPPCP

SEQ ID NO: 55 - protein, 13 aa, synthetic construct
  REGION 1..13: note = Description of Artificial Sequence: Synthetic peptide
ASTKGPSVFPLAP

SEQ ID NO: 56 - protein, 6 aa, synthetic construct
  REGION 1..6: note = Description of Artificial Sequence: Synthetic peptide
ASTKGP

SEQ ID NO: 57 - protein, 12 aa, synthetic construct
  REGION 1..12: note = Description of Artificial Sequence: Synthetic peptide
TVAAPSVFIFPP

SEQ ID NO: 58 - protein, 5 aa, synthetic construct
  REGION 1..5: note = Description of Artificial Sequence: Synthetic peptide
TVAAP

SEQ ID NO: 59 - protein, 16 aa, synthetic construct
  REGION 1..16: note = Description of Artificial Sequence: Synthetic peptide
AKTTPKLEEGEFSEAR

SEQ ID NO: 60 - protein, 17 aa, synthetic construct
  REGION 1..17: note = Description of Artificial Sequence: Synthetic peptide
AKTTPKLEEGEFSEARV

SEQ ID NO: 61 - protein, 9 aa, synthetic construct
  REGION 1..9: note = Description of Artificial Sequence: Synthetic peptide
AKTTPKLGG

SEQ ID NO: 62 - protein, 10 aa, synthetic construct
  REGION 1..10: note = Description of Artificial Sequence: Synthetic peptide
SAKTTPKLGG

SEQ ID NO: 63 - protein, 6 aa, synthetic construct
  REGION 1..6: note = Description of Artificial Sequence: Synthetic peptide
SAKTTP

SEQ ID NO: 64 - protein, 6 aa, synthetic construct
  REGION 1..6: note = Description of Artificial Sequence: Synthetic peptide
RADAAP

SEQ ID NO: 65 - protein, 9 aa, synthetic construct
  REGION 1..9: note = Description of Artificial Sequence: Synthetic peptide
RADAAPTVS

SEQ ID NO: 66 - protein, 12 aa, synthetic construct
  REGION 1..12: note = Description of Artificial Sequence: Synthetic peptide
RADAAAAGGPGS

SEQ ID NO: 67 - protein, 27 aa, synthetic construct
  REGION 1..27: note = Description of Artificial Sequence: Synthetic peptide
RADAAAAGGGGSGGGGSGGGGSGGGGS

SEQ ID NO: 68 - protein, 18 aa, synthetic construct
  REGION 1..18: note = Description of Artificial Sequence: Synthetic peptide
SAKTTPKLEEGEFSEARV

SEQ ID NO: 69 - protein, 5 aa, synthetic construct
  REGION 1..5: note = Description of Artificial Sequence: Synthetic peptide
ADAAP

SEQ ID NO: 70 - protein, 12 aa, synthetic construct
  REGION 1..12: note = Description of Artificial Sequence: Synthetic peptide
ADAAPTVSIFPP

SEQ ID NO: 71 - protein, 6 aa, synthetic construct
  REGION 1..6: note = Description of Artificial Sequence: Synthetic peptide
QPKAAP

SEQ ID NO: 72 - protein, 13 aa, synthetic construct
  REGION 1..13: note = Description of Artificial Sequence: Synthetic peptide
QPKAAPSVTLFPP

SEQ ID NO: 73 - protein, 6 aa, synthetic construct
  REGION 1..6: note = Description of Artificial Sequence: Synthetic peptide
AKTTPP

SEQ ID NO: 74 - protein, 13 aa, synthetic construct
  REGION 1..13: note = Description of Artificial Sequence: Synthetic peptide
AKTTPPSVTPLAP

SEQ ID NO: 75 - protein, 6 aa, synthetic construct
  REGION 1..6: note = Description of Artificial Sequence: Synthetic peptide
AKTTAP

SEQ ID NO: 76 - protein, 13 aa, synthetic construct
  REGION 1..13: note = Description of Artificial Sequence: Synthetic peptide
AKTTAPSVYPLAP

SEQ ID NO: 77 - protein, 15 aa, synthetic construct
  REGION 1..15: note = Description of Artificial Sequence: Synthetic peptide
GGGGSGGGGSGGGGS

SEQ ID NO: 78 - protein, 15 aa, synthetic construct
  REGION 1..15: note = Description of Artificial Sequence: Synthetic peptide
GENKVEYAPALMALS

SEQ ID NO: 79 - protein, 15 aa, synthetic construct
  REGION 1..15: note = Description of Artificial Sequence: Synthetic peptide
GPAKELTPLKEAKVS

SEQ ID NO: 80 - protein, 15 aa, synthetic construct
  REGION 1..15: note = Description of Artificial Sequence: Synthetic peptide
GHEAAAVMQVQYPAS

SEQ ID NO: 81 - protein, 24 aa, synthetic construct
  REGION 1..24: note = Description of Artificial Sequence: Synthetic peptide
TVAAPSVFIFPPTVAAPSVFIFPP

SEQ ID NO: 82 - protein, 26 aa, synthetic construct
  REGION 1..26: note = Description of Artificial Sequence: Synthetic peptide
ASTKGPSVFPLAPASTKGPSVFPLAP

SEQ ID NO: 83 - protein, 692 aa, Homo sapiens
MELLPLWLCLGFHFLTVGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLYLVLILPSCLTLLVACTVIVLTFKKPLLQVIKSRCHWSSVY

SEQ ID NO: 84 - protein, 660 aa, synthetic construct
  REGION 1..660: note = Description of Artificial Sequence: Synthetic polypeptide
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLHHHHHH

SEQ ID NO: 85-100 - skipped (16 entries)

SEQ ID NO: 101 - protein, 689 aa, synthetic construct
  REGION 1..689: note = Description of Artificial Sequence: Synthetic polypeptide
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLIIILTFILVSAILLTTLAACCCVRRQKFNQQYKA

SEQ ID NO: 102-257 - skipped (156 entries)

SEQ ID NO: 258 - protein, 21 aa, Homo sapiens
SQGEVPPGPLPEAVLALYNST

SEQ ID NO: 259 - protein, 7 aa, Homo sapiens
LREAVPE

SEQ ID NO: 260 - protein, 8 aa, Homo sapiens
YHANFCLG

SEQ ID NO: 261 - protein, 24 aa, Homo sapiens
SPPSQGEVPPGPLPEAVLALYNST

SEQ ID NO: 262 - protein, 17 aa, Homo sapiens
WKWIHEPKGYHANFCLG

SEQ ID NO: 263 - protein, 249 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 264 - protein, 45 aa, Homo sapiens
LSTCKTIDMELVKRKRIEAIRGQILSKLRLASPPSQGEVPPGPLP

SEQ ID NO: 265 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 266 - protein, 4 aa, Homo sapiens
RHRR

SEQ ID NO: 267 - protein, 204 aa, Homo sapiens
EAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 268 - protein, 29 aa, Homo sapiens
CVRQLYIDFRKDLGWKWIHEPKGYHANFC

SEQ ID NO: 269 - protein, 35 aa, Homo sapiens
CVPQALEPLPIVYYVGRKPKVEQLSNMIVRSCKCS

SEQ ID NO: 270 - protein, 15 aa, Homo sapiens
LASPPSQGEVPPGPL

SEQ ID NO: 271 - protein, 27 aa, Homo sapiens
LASPPSQGEVPPGPLPEAVLALYNSTR

SEQ ID NO: 272 - protein, 29 aa, Homo sapiens
LSTCKTIDMELVKRKRIEAIRGQILSKLR

SEQ ID NO: 273 - protein, 10 aa, Homo sapiens
AVLALYNSTR

SEQ ID NO: 274 - protein, 20 aa, Homo sapiens
NGFTTGRRGDLATIHGMNRP

SEQ ID NO: 275 - protein, 13 aa, Homo sapiens
CSCDSRDNTLQVD

SEQ ID NO: 276 - protein, 8 aa, synthetic construct
  REGION 1..8: note = Description of Artificial Sequence: Synthetic peptide
GFTFADYA

SEQ ID NO: 277 - protein, 8 aa, synthetic construct
  REGION 1..8: note = Description of Artificial Sequence: Synthetic peptide
  MOD_RES 6: note = A or K
ISGSGXAT

SEQ ID NO: 278 - protein, 9 aa, synthetic construct
  REGION 1..9: note = Description of Artificial Sequence: Synthetic peptide
  MOD_RES 5: note = H, D or Q
  MOD_RES 8: note = F or Y
VSSGXWDXD

SEQ ID NO: 279 - protein, 6 aa, synthetic construct
  REGION 1..6: note = Description of Artificial Sequence: Synthetic peptide
QSISSY

SEQ ID NO: 280 - skipped

SEQ ID NO: 281 - protein, 9 aa, synthetic construct
  REGION 1..9: note = Description of Artificial Sequence: Synthetic peptide
  MOD_RES 5: note = T or G
QQTYXVPLT

SEQ ID NO: 282 - protein, 8 aa, synthetic construct
  REGION 1..8: note = Description of Artificial Sequence: Synthetic peptide
ISGSGAAT

SEQ ID NO: 283 - protein, 9 aa, synthetic construct
  REGION 1..9: note = Description of Artificial Sequence: Synthetic peptide
  MOD_RES 5: note = H or Q
  MOD_RES 8: note = Y or F
VSSGXWDXD

SEQ ID NO: 284 - protein, 7 aa, synthetic construct
  REGION 1..7: note = Description of Artificial Sequence: Synthetic peptide
AASGLES

SEQ ID NO: 285 - protein, 9 aa, synthetic construct
  REGION 1..9: note = Description of Artificial Sequence: Synthetic peptide
QQTYGVPLT

SEQ ID NO: 286 - protein, 14 aa, Homo sapiens
ASPPSQGEVPPGPL

SEQ ID NO: 287 - protein, 9 aa, synthetic construct
  REGION 1..9: note = Description of Artificial Sequence: Synthetic peptide
VSSGHWDYD

SEQ ID NO: 288 - protein, 8 aa, synthetic construct
  REGION 1..8: note = Description of Artificial Sequence: Synthetic peptide
ISGSGKAT

SEQ ID NO: 289 - protein, 9 aa, synthetic construct
  REGION 1..9: note = Description of Artificial Sequence: Synthetic peptide
VSSGHWDFD

SEQ ID NO: 290 - protein, 9 aa, synthetic construct
  REGION 1..9: note = Description of Artificial Sequence: Synthetic peptide
VSSGDWDYD

SEQ ID NO: 291 - protein, 9 aa, synthetic construct
  REGION 1..9: note = Description of Artificial Sequence: Synthetic peptide
VSSGQWDYD

SEQ ID NO: 292 - protein, 7 aa, synthetic construct
  REGION 1..7: note = Description of Artificial Sequence: Synthetic peptide
AASNLES

SEQ ID NO: 293 - protein, 7 aa, synthetic construct
  REGION 1..7: note = Description of Artificial Sequence: Synthetic peptide
AASVNES

SEQ ID NO: 294 - protein, 7 aa, synthetic construct
  REGION 1..7: note = Description of Artificial Sequence: Synthetic peptide
AASNEET

SEQ ID NO: 295 - protein, 7 aa, synthetic construct
  REGION 1..7: note = Description of Artificial Sequence: Synthetic peptide
AASNLQS

SEQ ID NO: 296 - protein, 9 aa, synthetic construct
  REGION 1..9: note = Description of Artificial Sequence: Synthetic peptide
QQTYTVPLT

SEQ ID NO: 297 - protein, 119 aa, synthetic construct
  REGION 1..119: note = Description of Artificial Sequence: Synthetic polypeptide
EVQLLESGGGLVQPGGSLRLSCAASGFTFADYAMTWVRQAPGKGLEWVSAISGSGAATYFADSVKGRFTISRDNSKNTLYLQMNSLRAEDTAVYYCARVSSGHWDYDYWGQGTLVTVSS

SEQ ID NO: 298 - protein, 107 aa, synthetic construct
  REGION 1..107: note = Description of Artificial Sequence: Synthetic peptide
DIQLTQSPSSLSASVGDRVTITCRASQSISSYLNWYQQKPGKAPKLLIYAASGLESGVPSRFSGSGSGTDFTLTISSLQPEDFATYYCQQTYGVPLTFGGGTKVEIK

SEQ ID NO: 299 - protein, 119 aa, synthetic construct
  REGION 1..119: note = Description of Artificial Sequence: Synthetic polypeptide
EVQLLESGGGLVQPGGSLRLSCAASGFTFADYAMTWVRQAPGKGLEWVSAISGSGAATYFADSVKGRFTISRDNSKNTLYLQMNSLRAEDTAVYYCARVSSGHWDFDYWGQGTLVTVSS

SEQ ID NO: 300 - protein, 107 aa, synthetic construct
  REGION 1..107: note = Description of Artificial Sequence: Synthetic polypeptide
DIQLTQSPSSLSASVGDRVTITCRASQSISSYLNWYQQKPGKAPKLLIYAASNLQSGVPSRFSGSGSGTDFTLTISSLQPEDFATYYCQQTYTVPLTFGGGTKVEIK

SEQ ID NO: 301 - protein, 6 aa, unidentified
  REGION 1..6: note = Description of Unknown: TGFBETA1 sequence
LSKLRL

SEQ ID NO: 302 - protein, 6 aa, synthetic construct
  REGION 1..6: note = Description of Artificial Sequence: Synthetic 6xHis tag
HHHHHH

SEQ ID NO: 303 - protein, 7 aa, synthetic construct
  REGION 1..7: note = Description of Artificial Sequence: Synthetic peptide
  MOD_RES 4: note = G or N
AASXLES

SEQ ID NO: 304 - protein, 20 aa, Homo sapiens
EDYPEPEEVPPEVISIYNST

SEQ ID NO: 305 - protein, 4 aa, Homo sapiens
MEKN

SEQ ID NO: 306 - protein, 8 aa, Homo sapiens
YNANFCAG

SEQ ID NO: 307 - protein, 19 aa, Homo sapiens
EPTVMTHVPYQVLALYNST

SEQ ID NO: 308 - protein, 4 aa, Homo sapiens
VEKN

SEQ ID NO: 309 - protein, 8 aa, Homo sapiens
YYANFCSG

SEQ ID NO: 310 - protein, 23 aa, Homo sapiens
SPPEDYPEPEEVPPEVISIYNST

SEQ ID NO: 311 - protein, 17 aa, Homo sapiens
WKWIHEPKGYNANFCAG

SEQ ID NO: 312 - protein, 22 aa, Homo sapiens
SPPEPTVMTHVPYQVLALYNST

SEQ ID NO: 313 - protein, 17 aa, Homo sapiens
WKWVHEPKGYYANFCSG

SEQ ID NO: 314 - protein, 23 aa, Homo sapiens
KLRLASPPSQGEVPPGPLPEAVL

SEQ ID NO: 315 - protein, 22 aa, Homo sapiens
KLKLTSPPEDYPEPEEVPPEVI

SEQ ID NO: 316 - protein, 21 aa, Homo sapiens
KLRLTSPPEPTVMTHVPYQVL

SEQ ID NO: 317-1000 - skipped (684 entries)

SEQ ID NO: 1001 - protein, 8 aa, synthetic construct
  REGION 1..8: note = Description of Artificial Sequence: Synthetic peptide
GFTFSSFS

SEQ ID NO: 1002 - protein, 8 aa, synthetic construct
  REGION 1..8: note = Description of Artificial Sequence: Synthetic peptide
ISPSADTI

SEQ ID NO: 1003 - protein, 13 aa, synthetic construct
  REGION 1..13: note = Description of Artificial Sequence: Synthetic peptide
ARGVLDYGDMLMP

SEQ ID NO: 1004 - protein, 6 aa, synthetic construct
  REGION 1..6: note = Description of Artificial Sequence: Synthetic peptide
QDITNY

SEQ ID NO: 1005 - skipped

SEQ ID NO: 1006 - protein, 10 aa, synthetic construct
  REGION 1..10: note = Description of Artificial Sequence: Synthetic peptide
QQADNHPPWT

SEQ ID NO: 1007 - protein, 120 aa, synthetic construct
  REGION 1..120: note = Description of Artificial Sequence: Synthetic polypeptide
EVQLVESGGGLVQPGGSLRLSCTASGFTFSSFSMDWVRQAPGKGLEWVSYISPSADTIYYADSVKGRFTISRDNAKNTLYLQMNSLRAEDTAVYYCARGVLDYGDMLMPWGQGTLVTVSS

SEQ ID NO: 1008 - protein, 108 aa, synthetic construct
  REGION 1..108: note = Description of Artificial Sequence: Synthetic polypeptide
DIQMTQSPSSLSASVGDRVTITCQASQDITNYLNWYQQKPGKAPKLLIYDASNLETGVPSRFSGSGSGTDFTFTISSLQPEDIATYYCQQADNHPPWTFGGGTKVEIK

SEQ ID NO: 1009 - protein, 446 aa, synthetic construct
  REGION 1..446: note = Description of Artificial Sequence: Synthetic polypeptide
EVQLVESGGGLVQPGGSLRLSCTASGFTFSSFSMDWVRQAPGKGLEWVSYISPSADTIYYADSVKGRFTISRDNAKNTLYLQMNSLRAEDTAVYYCARGVLDYGDMLMPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 1010 - DNA, 1338 bp, synthetic construct
  misc_feature 1..1338: note = Description of Artificial Sequence: Synthetic polynucleotide
  source 1..1338: mol_type = other DNA
gaggtgcagctggtggagtctgggggaggcttggtacagcctggggggtccctgagactctcctgtacagcctctggattcaccttcagtagcttcagcatggactgggtccgccaggctccagggaaggggctggagtgggtttcatacattagtcccagtgcagacaccatatactacgcagactctgtgaagggccgattcaccatctccagagacaatgccaagaacacgctgtatctgcaaatgaacagcctgagagccgaggacacggcggtgtactactgcgccagaggggtgctcgactacggagacatgttaatgccatggggccagggaaccctggtcaccgtctcctcagcgtcgaccaagggcccttccgtgttccctctggccccttgctcccggtccacctccgagtccaccgccgctctgggctgtctggtgaaggactacttccctgagcctgtgaccgtgagctggaactctggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgggtggagtccaagtacggccctccttgccctccctgccctgcccctgagttcctgggcggaccctccgtgttcctgttccctcctaagcctaaggacaccctgatgatctcccggacccctgaggtgacctgcgtggtggtggacgtgtcccaggaagatcctgaggtccagttcaattggtacgtggatggcgtggaggtgcacaacgccaagaccaagcctcgggaggaacagttcaactccacctaccgggtggtgtctgtgctgaccgtgctgcaccaggactggctgaacggcaaggaatacaagtgcaaggtcagcaacaagggcctgccctcctccatcgagaaaaccatctccaaggccaagggccagcctcgcgagcctcaggtgtacaccctgcctcctagccaggaagagatgaccaagaatcaggtgtccctgacatgcctggtgaagggcttctacccttccgatatcgccgtggagtgggagagcaacggccagccagagaacaactacaagaccacccctcctgtgctggactccgacggctccttcttcctgtactccaggctgaccgtggacaagtcccggtggcaggaaggcaacgtcttttcctgctccgtgatgcacgaggccctgcacaaccactacacccagaagtccctgtccctgtctctgggc

SEQ ID NO: 1011 - protein, 215 aa, synthetic construct
  REGION 1..215: note = Description of Artificial Sequence: Synthetic polypeptide
DIQMTQSPSSLSASVGDRVTITCQASQDITNYLNWYQQKPGKAPKLLIYDASNLETGVPSRFSGSGSGTDFTFTISSLQPEDIATYYCQQADNHPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1012 - DNA, 645 bp, synthetic construct
  misc_feature 1..645: note = Description of Artificial Sequence: Synthetic polynucleotide
  source 1..645: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcagcaggccgacaatcaccctccttggacttttggcggagggaccaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 1013 - protein, 361 aa, Homo sapiens; identical to SEQ ID NO: 24

SEQ ID NO: 1014 - protein, 361 aa, synthetic construct; identical to SEQ ID NO: 25
  REGION 1..361: note = Description of Artificial Sequence: Synthetic polypeptide

SEQ ID NO: 1015 - protein, 360 aa, synthetic construct; identical to SEQ ID NO: 26
  REGION 1..360: note = Description of Artificial Sequence: Synthetic polypeptide

SEQ ID NO: 1016 - protein, 360 aa, synthetic construct; identical to SEQ ID NO: 27
  REGION 1..360: note = Description of Artificial Sequence: Synthetic polypeptide

SEQ ID NO: 1017 - protein, 395 aa, Homo sapiens; identical to SEQ ID NO: 28

SEQ ID NO: 1018 - protein, 395 aa, synthetic construct; identical to SEQ ID NO: 29
  REGION 1..395: note = Description of Artificial Sequence: Synthetic polypeptide

SEQ ID NO: 1019 - protein, 394 aa, synthetic construct; identical to SEQ ID NO: 30
  REGION 1..394: note = Description of Artificial Sequence: Synthetic polypeptide

SEQ ID NO: 1020 - protein, 394 aa, synthetic construct; identical to SEQ ID NO: 31
  REGION 1..394: note = Description of Artificial Sequence: Synthetic polypeptide

SEQ ID NO: 1021 - protein, 392 aa, Homo sapiens; identical to SEQ ID NO: 32

SEQ ID NO: 1022 - protein, 392 aa, synthetic construct; identical to SEQ ID NO: 33
  REGION 1..392: note = Description of Artificial Sequence: Synthetic polypeptide

SEQ ID NO: 1023 - protein, 391 aa, synthetic construct; identical to SEQ ID NO: 34
  REGION 1..391: note = Description of Artificial Sequence: Synthetic polypeptide

SEQ ID NO: 1024 - protein, 391 aa, synthetic construct; identical to SEQ ID NO: 35
  REGION 1..391: note = Description of Artificial Sequence: Synthetic polypeptide

SEQ ID NO: 1025 - protein, 361 aa, Mus musculus; identical to SEQ ID NO: 36

SEQ ID NO: 1026 - protein, 361 aa, Macaca fascicularis; identical to SEQ ID NO: 37

SEQ ID NO: 1027 - protein, 249 aa, synthetic construct; identical to SEQ ID NO: 38
  REGION 1..249: note = Description of Artificial Sequence: Synthetic polypeptide

SEQ ID NO: 1028 - protein, 249 aa, synthetic construct; identical to SEQ ID NO: 39
  REGION 1..249: note = Description of Artificial Sequence: Synthetic polypeptide

SEQ ID NO: 1029 - protein, 360 aa, synthetic construct; identical to SEQ ID NO: 40
  REGION 1..360: note = Description of Artificial Sequence: Synthetic polypeptide

SEQ ID NO: 1030 - protein, 361 aa, synthetic construct; identical to SEQ ID NO: 41
  REGION 1..361: note = Description of Artificial Sequence: Synthetic polypeptide

SEQ ID NO: 1031 - protein, 361 aa, synthetic construct; identical to SEQ ID NO: 42
  REGION 1..361: note = Description of Artificial Sequence: Synthetic polypeptide

SEQ ID NO: 1032 - protein, 360 aa, synthetic construct; identical to SEQ ID NO: 43
  REGION 1..360: note = Description of Artificial Sequence: Synthetic polypeptide

SEQ ID NO: 1033 - protein, 1260 aa, Macaca fascicularis; identical to SEQ ID NO: 44

SEQ ID NO: 1034 - protein, 1228 aa, Mus musculus; identical to SEQ ID NO: 45

SEQ ID NO: 1035 - protein, 1373 aa, Macaca fascicularis; identical to SEQ ID NO: 46

SEQ ID NO: 1036 - protein, 1374 aa, Mus musculus; identical to SEQ ID NO: 47

SEQ ID NO: 1037 - protein, 646 aa, Mus musculus; identical to SEQ ID NO: 48

SEQ ID NO: 1038 - protein, 611 aa, Mus musculus; identical to SEQ ID NO: 49

SEQ ID NO: 1039 - protein, 1375 aa, unidentified; identical to SEQ ID NO: 50
  REGION 1..1375: note = Description of Unknown: LTBP1S sequence

SEQ ID NO: 1040 - protein, 1260 aa, unidentified; identical to SEQ ID NO: 51
  REGION 1..1260: note = Description of Unknown: LTBP3 sequence

SEQ ID NO: 1041 - protein, 645 aa, unidentified; identical to SEQ ID NO: 52
  REGION 1..645: note = Description of Unknown: GARP sequence

SEQ ID NO: 1042 - protein, 610 aa, unidentified; identical to SEQ ID NO: 53
  REGION 1..610: note = Description of Unknown: sGARP sequence

SEQ ID NO: 1043 - protein, 5 aa, synthetic construct
  REGION 1..5: note = Description of Artificial Sequence: Synthetic peptide
CPPCP

SEQ ID NO: 1044 - protein, 13 aa, synthetic construct
  REGION 1..13: note = Description of Artificial Sequence: Synthetic peptide
ASTKGPSVFPLAP

SEQ ID NO: 1045 - protein, 6 aa, synthetic construct
  REGION 1..6: note = Description of Artificial Sequence: Synthetic peptide
ASTKGP

SEQ ID NO: 1046 - protein, 12 aa, synthetic construct
  REGION 1..12: note = Description of Artificial Sequence: Synthetic peptide
TVAAPSVFIFPP

SEQ ID NO: 1047 - protein, 5 aa, synthetic construct
  REGION 1..5: note = Description of Artificial Sequence: Synthetic peptide
TVAAP

SEQ ID NO: 1048 - protein, 16 aa, synthetic construct
  REGION 1..16: note = Description of Artificial Sequence: Synthetic peptide
AKTTPKLEEGEFSEAR

SEQ ID NO: 1049 - protein, 17 aa, synthetic construct
  REGION 1..17: note = Description of Artificial Sequence: Synthetic peptide
AKTTPKLEEGEFSEARV

SEQ ID NO: 1050 - protein, 9 aa, synthetic construct
  REGION 1..9: note = Description of Artificial Sequence: Synthetic peptide
AKTTPKLGG

SEQ ID NO: 1051 - protein, 10 aa, synthetic construct
  REGION 1..10: note = Description of Artificial Sequence: Synthetic peptide
SAKTTPKLGG

SEQ ID NO: 1052 - protein, 6 aa, synthetic construct
  REGION 1..6: note = Description of Artificial Sequence: Synthetic peptide
SAKTTP

SEQ ID NO: 1053 - protein, 6 aa, synthetic construct
  REGION 1..6: note = Description of Artificial Sequence: Synthetic peptide
RADAAP

SEQ ID NO: 1054 - protein, 9 aa, synthetic construct
  REGION 1..9: note = Description of Artificial Sequence: Synthetic peptide
RADAAPTVS

SEQ ID NO: 1055 - protein, 12 aa, synthetic construct
  REGION 1..12: note = Description of Artificial Sequence: Synthetic peptide
RADAAAAGGPGS

SEQ ID NO: 1056 - protein, 27 aa, synthetic construct
  REGION 1..27: note = Description of Artificial Sequence: Synthetic peptide
RADAAAAGGGGSGGGGSGGGGSGGGGS

SEQ ID NO: 1057 - protein, 18 aa, synthetic construct
  REGION 1..18: note = Description of Artificial Sequence: Synthetic peptide
SAKTTPKLEEGEFSEARV

SEQ ID NO: 1058 - protein, 5 aa, synthetic construct
  REGION 1..5: note = Description of Artificial Sequence: Synthetic peptide
ADAAP

SEQ ID NO: 1059 - protein, 12 aa, synthetic construct
  REGION 1..12: note = Description of Artificial Sequence: Synthetic peptide
ADAAPTVSIFPP

SEQ ID NO: 1060 - protein, 6 aa, synthetic construct
  REGION 1..6: note = Description of Artificial Sequence: Synthetic peptide
QPKAAP

SEQ ID NO: 1061 - protein, 13 aa, synthetic construct
  REGION 1..13: note = Description of Artificial Sequence: Synthetic peptide
QPKAAPSVTLFPP

SEQ ID NO: 1062 - protein, 6 aa, synthetic construct
  REGION 1..6: note = Description of Artificial Sequence: Synthetic peptide
AKTTPP

SEQ ID NO: 1063 - protein, 13 aa, synthetic construct
  REGION 1..13: note = Description of Artificial Sequence: Synthetic peptide
AKTTPPSVTPLAP

SEQ ID NO: 1064 - protein, 6 aa, synthetic construct
  REGION 1..6: note = Description of Artificial Sequence: Synthetic peptide
AKTTAP

SEQ ID NO: 1065 - skipped

SEQ ID NO: 1066 - protein, 15 aa, synthetic construct
  REGION 1..15: note = Description of Artificial Sequence: Synthetic peptide
GGGGSGGGGSGGGGS

SEQ ID NO: 1067 - protein, 15 aa, synthetic construct
  REGION 1..15: note = Description of Artificial Sequence: Synthetic peptide
GENKVEYAPALMALS

SEQ ID NO: 1068 - protein, 15 aa, synthetic construct
  REGION 1..15: note = Description of Artificial Sequence: Synthetic peptide
GPAKELTPLKEAKVS

SEQ ID NO: 1069 - protein, 15 aa, synthetic construct
  REGION 1..15: note = Description of Artificial Sequence: Synthetic peptide
GHEAAAVMQVQYPAS

SEQ ID NO: 1070 - protein, 24 aa, synthetic construct
  REGION 1..24: note = Description of Artificial Sequence: Synthetic peptide
TVAAPSVFIFPPTVAAPSVFIFPP

SEQ ID NO: 1071 - protein, 26 aa, synthetic construct
  REGION 1..26: note = Description of Artificial Sequence: Synthetic peptide
ASTKGPSVFPLAPASTKGPSVFPLAP

SEQ ID NO: 1072 - protein, 692 aa, Homo sapiens; identical to SEQ ID NO: 83

SEQ ID NO: 1073 - protein, 660 aa, synthetic construct; identical to SEQ ID NO: 84
  REGION 1..660: note = Description of Artificial Sequence: Synthetic polypeptide

SEQ ID NO: 1074 - protein, 689 aa, synthetic construct; identical to SEQ ID NO: 101
  REGION 1..689: note = Description of Artificial Sequence: Synthetic polypeptide

SEQ ID NO: 1075 - protein, 5 aa, synthetic construct
  REGION 1..5: note = Description of Artificial Sequence: Synthetic peptide
SFSMD

SEQ ID NO: 1076 - protein, 17 aa, synthetic construct
  REGION 1..17: note = Description of Artificial Sequence: Synthetic peptide
YISPSADTIYYADSVKG

SEQ ID NO: 1077 - protein, 11 aa, synthetic construct
  REGION 1..11: note = Description of Artificial Sequence: Synthetic peptide
GVLDYGDMLMP

SEQ ID NO: 1078 - protein, 11 aa, synthetic construct
  REGION 1..11: note = Description of Artificial Sequence: Synthetic peptide
QASQDITNYLN

SEQ ID NO: 1079 - protein, 7 aa, synthetic construct
  REGION 1..7: note = Description of Artificial Sequence: Synthetic peptide
DASNLET

SEQ ID NO: 1080 - protein, 9 aa, synthetic construct
  REGION 1..9: note = Description of Artificial Sequence: Synthetic peptide
FTFSSFSMD

SEQ ID NO: 1081 - protein, 9 aa, synthetic construct
  REGION 1..9: note = Description of Artificial Sequence: Synthetic peptide
FTFSSYSMN

SEQ ID NO: 1082 - protein, 17 aa, synthetic construct
  REGION 1..17: note = Description of Artificial Sequence: Synthetic peptide
YISSSSSTIYYADSVKG

SEQ ID NO: 1083 - protein, 13 aa, synthetic construct
  REGION 1..13: note = Description of Artificial Sequence: Synthetic peptide
ARGVLDYGDMLDP

SEQ ID NO: 1084 - protein, 17 aa, synthetic construct
  REGION 1..17: note = Description of Artificial Sequence: Synthetic peptide
YISPDASTIYYADSVKG

SEQ ID NO: 1085 - skipped

SEQ ID NO: 1086 - protein, 17 aa, synthetic construct
  REGION 1..17: note = Description of Artificial Sequence: Synthetic peptide
YIHSDASTIYYADSVKG

SEQ ID NO: 1087 - protein, 9 aa, synthetic construct
  REGION 1..9: note = Description of Artificial Sequence: Synthetic peptide
FTFSSFSMN

SEQ ID NO: 1088 - protein, 9 aa, synthetic construct
  REGION 1..9: note = Description of Artificial Sequence: Synthetic peptide
FTFGSFSMN

SEQ ID NO: 1089 - skipped

SEQ ID NO: 1090 - protein, 9 aa, synthetic construct
  REGION 1..9: note = Description of Artificial Sequence: Synthetic peptide
FTFSFYAMN

SEQ ID NO: 1091 - protein, 13 aa, synthetic construct
  REGION 1..13: note = Description of Artificial Sequence: Synthetic peptide
VRGVLDYGDMLDP

SEQ ID NO: 1092 - protein, 9 aa, synthetic construct
  REGION 1..9: note = Description of Artificial Sequence: Synthetic peptide
FTFSSFAMN

SEQ ID NO: 1093 - protein, 17 aa, synthetic construct
  REGION 1..17: note = Description of Artificial Sequence: Synthetic peptide
YISPDASTIYYAGSVKG

SEQ ID NO: 1094 - protein, 13 aa, synthetic construct
  REGION 1..13: note = Description of Artificial Sequence: Synthetic peptide
VRAVLDYGDMLDP

SEQ ID NO: 1095 - protein, 13 aa, synthetic construct
  REGION 1..13: note = Description of Artificial Sequence: Synthetic peptide
ARGTLDYGDMLDP

SEQ ID NO: 1096 - protein, 13 aa, synthetic construct
  REGION 1..13: note = Description of Artificial Sequence: Synthetic peptide
ARAVLDYGDMLDP

SEQ ID NO: 1097 - protein, 13 aa, synthetic construct
  REGION 1..13: note = Description of Artificial Sequence: Synthetic peptide
ARGVWDMGDMLDP

SEQ ID NO: 1098 - protein, 13 aa, synthetic construct
  REGION 1..13: note = Description of Artificial Sequence: Synthetic peptide
AHGVLDYGDMLDP

SEQ ID NO: 1099 - protein, 9 aa, synthetic construct
  REGION 1..9: note = Description of Artificial Sequence: Synthetic peptide
FTFAFYSMN

SEQ ID NO: 1100 - protein, 120 aa, synthetic construct
  REGION 1..120: note = Description of Artificial Sequence: Synthetic polypeptide
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGVLDYGDMLDPWGQGTLVTVSS

SEQ ID NO: 1101 - protein, 120 aa, synthetic construct
  REGION 1..120: note = Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 1102 - protein, 120 aa, synthetic construct; identical to SEQ ID NO: 1101
  REGION 1..120: note = Description of Artificial Sequence: Synthetic polypeptide

SEQ ID NO: 1103 - protein, 120 aa, synthetic construct
  REGION 1..120: note = Description of Artificial Sequence: Synthetic polypeptide
EVQLVESGGGLVQPGGSLRLSCAASGFTFGSFSMNWVRQAPGKGLEWVSYIHSDASTIYYADSVKGRFTISRDNAKNSLYLQMNSLRAEDTAVYYCARGVLDYGDMLDPWGQGTLVTVSS

SEQ ID NO: 1104 - protein, 120 aa, synthetic construct
  REGION 1..120: note = Description of Artificial Sequence: Synthetic polypeptide
EVQLVESGGGLVQPGGSLRLSCAASGFTFSSFSMNWVRQAPGKGLEWVSYISPSADTIYYADSVKGRFTISRDNAKNTLYLQMNSLRAEDTAVYYCARGVLDYGDMLDPWGQGTLVTVSS

SEQ ID NO: 1105 - protein, 120 aa, synthetic construct
  REGION 1..120: note = Description of Artificial Sequence: Synthetic polypeptide
EVQLVESGGGLVQPGRSLRLSCAASGFTFSSFAMYWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 1106 - protein, 120 aa, synthetic construct
  REGION 1..120: note = Description of Artificial Sequence: Synthetic polypeptide
EVQLVESGGGLVQPGGSLRLSCAASGFTFGSFSMDWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 1107 - protein, 120 aa, synthetic construct
  REGION 1..120: note = Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNTLYLQMNSLRAEDTAVYYCARGVLDYGDMLDPWGQGTLVTVSS

SEQ ID NO: 1108 - protein, 120 aa, synthetic construct
  REGION 1..120: note = Description of Artificial Sequence: Synthetic polypeptide
EVQLVESGGGLVQPGGSLRLSCAASGFTFSFYAMNWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 1109 - protein, 120 aa, synthetic construct
  REGION 1..120: note = Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNSLYLQMNSLRAEDTAVYYCVRGVLDYGDMLDPWGQGTLVTVSS

SEQ ID NO: 1110 - protein, 120 aa, synthetic construct
  REGION 1..120: note = Description of Artificial Sequence: Synthetic polypeptide
EVQLVESGGGLVQPGRSLRLSCAASGFTFSSFAMNWVRQAPGKGLEWVSYISPDASTIYYAGSVKGRFTISRDNAKNSLYLQMNSLRAEDTAVYYCVRAVLDYGDMLDPWGQGTLVTVSS

SEQ ID NO: 1111 - protein, 120 aa, synthetic construct
  REGION 1..120: note = Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNSLYLQMNSLRAEDTAVYYCARGTLDYGDMLDPWGQGTLVTVSS

SEQ ID NO: 1112 - protein, 120 aa, synthetic construct
  REGION 1..120: note = Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNTLYLQMNSLRAEDTAVYYCARAVLDYGDMLDPWGQGTLVTVSS

SEQ ID NO: 1113 - protein, 120 aa, synthetic construct
  REGION 1..120: note = Description of Artificial Sequence: Synthetic polypeptide
EVQLVESGGGLVQPGGSLRLSCAASGFTFSSFSMNWVRQAPGKGLEWVSYISPSADTIYYADSVKGRFTISRDNAKNTLYLQMNSLRAEDTAVYYCARGVWDMGDMLDPWGQGTLVTVSS

SEQ ID NO: 1114 - protein, 120 aa, synthetic construct
  REGION 1..120: note = Description of Artificial Sequence: Synthetic polypeptide
EVQLVESGGGLVQPGGSLRLSCAASGFTFSSFSMNWVRQAPGKGLEWVSYISPSADTIYYADSVKGRFTISRDNAKNTLYLQMNSLRAEDTAVYYCAHGVLDYGDMLDPWGQGTLVTVSS

SEQ ID NO: 1115 - protein, 120 aa, synthetic construct
  REGION 1..120: note = Description of Artificial Sequence: Synthetic polypeptide
EVQLVESGGGLVQPGGSLRLSCAASGFTFAFYSMNWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 1116 - protein, 9 aa, synthetic construct
  REGION 1..9: note = Description of Artificial Sequence: Synthetic peptide
  MOD_RES 4: note = S, G or A
  MOD_RES 5: note = S or F
  MOD_RES 6: note = F or Y
  MOD_RES 7: note = S or A
  MOD_RES 9: note = D, N or Y
FTFXXXXMX

SEQ ID NO: 1117 - protein, 17 aa, synthetic construct
  REGION 1..17: note = Description of Artificial Sequence: Synthetic peptide
  MOD_RES 3: note = S or H
  MOD_RES 4: note = P or S
  MOD_RES 5: note = S or D
  MOD_RES 7: note = D or S
  MOD_RES 13: note = D or G
YIXXXAXTIYYAXSVKG

SEQ ID NO: 1118 - protein, 13 aa, synthetic construct
  REGION 1..13: note = Description of Artificial Sequence: Synthetic peptide
  MOD_RES 1: note = A or V
  MOD_RES 3: note = G or A
  MOD_RES 4: note = V or T
  MOD_RES 5: note = L or W
  MOD_RES 7: note = Y or M
  MOD_RES 12: note = M or D
XRXXXDXGDMLXP

SEQ ID NO: 1119 - protein, 249 aa, Homo sapiens; identical to SEQ ID NO: 263

SEQ ID NO: 1120 - protein, 45 aa, Homo sapiens; identical to SEQ ID NO: 264

SEQ ID NO: 1121 - protein, 112 aa, Homo sapiens; identical to SEQ ID NO: 265

SEQ ID NO: 1122 - protein, 4 aa, Homo sapiens
RHRR

SEQ ID NO: 1123 - protein, 204 aa, Homo sapiens; identical to SEQ ID NO: 267

SEQ ID NO: 1124 - protein, 29 aa, Homo sapiens
CVRQLYIDFRKDLGWKWIHEPKGYHANFC

SEQ ID NO: 1125 - protein, 35 aa, Homo sapiens; identical to SEQ ID NO: 269

SEQ ID NO: 1126 - protein, 15 aa, Homo sapiens
LASPPSQGEVPPGPL

SEQ ID NO: 1127 - protein, 27 aa, Homo sapiens
LASPPSQGEVPPGPLPEAVLALYNSTR

SEQ ID NO: 1128 - protein, 29 aa, Homo sapiens
LSTCKTIDMELVKRKRIEAIRGQILSKLR

SEQ ID NO: 1129 - protein, 10 aa, Homo sapiens
AVLALYNSTR

SEQ ID NO: 1130 - protein, 20 aa, Homo sapiens
NGFTTGRRGDLATIHGMNRP

SEQ ID NO: 1131 - protein, 13 aa, Homo sapiens
CSCDSRDNTLQVD

SEQ ID NO: 1132 - protein, 9 aa, Homo sapiens
LVKRKRIEA

SEQ ID NO: 1133 - protein, 11 aa, Homo sapiens
LASPPSQGEVP

SEQ ID NO: 1134 - protein, 8 aa, Homo sapiens
PGPLPEAV

SEQ ID NO: 1135 - protein, 8 aa, Homo sapiens
LALYNSTR

SEQ ID NO: 1136 - protein, 9 aa, Homo sapiens
REAVPEPVL

SEQ ID NO: 1137 - protein, 10 aa, Homo sapiens
YQKYSNNSWR

SEQ ID NO: 1138 - protein, 19 aa, Homo sapiens
RKDLGWKWIHEPKGYHANF

SEQ ID NO: 1139 - protein, 9 aa, Homo sapiens
LGPCPYIWS

SEQ ID NO: 1140 - protein, 8 aa, Homo sapiens
ALEPLPIV

SEQ ID NO: 1141 - protein, 10 aa, Homo sapiens
VGRKPKVEQL

SEQ ID NO: 1142 - protein, 23 aa, Homo sapiens
KLRLASPPSQGEVPPGPLPEAVL

SEQ ID NO: 1143 - protein, 8 aa, Homo sapiens
RKDLGWKW

SEQ ID NO: 1144 - protein, 11 aa, Homo sapiens
RKDLGWKWIHE

SEQ ID NO: 1145 - protein, 10 aa, Homo sapiens
HEPKGYHANF

SEQ ID NO: 1146 - protein, 21 aa, Homo sapiens
LRLASPPSQGEVPPGPLPEAV

SEQ ID NO: 1147 - protein, 26 aa, synthetic construct
  REGION 1..26: note = Description of Artificial Sequence: Synthetic peptide
EVQLVESGGGLVQPGGSLRLSCAASG

SEQ ID NO: 1148 - protein, 14 aa, synthetic construct
  REGION 1..14: note = Description of Artificial Sequence: Synthetic peptide
WVRQAPGKGLEWVS

SEQ ID NO: 1149 - protein, 30 aa, synthetic construct
  REGION 1..30: note = Description of Artificial Sequence: Synthetic polypeptide
RFTISRDNAKNSLYLQMNSLRAEDTAVYYC

SEQ ID NO: 1150 - protein, 11 aa, synthetic construct
  REGION 1..11: note = Description of Artificial Sequence: Synthetic peptide
WGQGTLVTVSS

SEQ ID NO: 1151 - protein, 23 aa, synthetic construct
  REGION 1..23: note = Description of Artificial Sequence: Synthetic peptide
DIQMTQSPSSLSASVGDRVTITC

SEQ ID NO: 1152 - protein, 15 aa, synthetic construct
  REGION 1..15: note = Description of Artificial Sequence: Synthetic peptide
WYQQKPGKAPKLLIY

SEQ ID NO: 1153 - protein, 32 aa, synthetic construct
  REGION 1..32: note = Description of Artificial Sequence: Synthetic polypeptide
GVPSRFSGSGSGTDFTFTISSLQPEDIATYYC

SEQ ID NO: 1154 - protein, 10 aa, synthetic construct
  REGION 1..10: note = Description of Artificial Sequence: Synthetic peptide
FGGGTKVEIK

SEQ ID NO: 1155-1164 - skipped (10 entries)

SEQ ID NO: 1165 - protein, 18 aa, Homo sapiens
SPPSQGEVPPGPLPEAVL

SEQ ID NO: 1166 - protein, 14 aa, Homo sapiens
WKWIHEPKGYHANF

SEQ ID NO: 1167 - protein, 9 aa, Homo sapiens
PGPLPEAVL

SEQ ID NO: 1168 - protein, 7 aa, synthetic construct
  REGION 1..7: note = Description of Artificial Sequence: Synthetic peptide
GFTFSSF

SEQ ID NO: 1169 - protein, 6 aa, synthetic construct
  REGION 1..6: note = Description of Artificial Sequence: Synthetic peptide
SPSADT

SEQ ID NO: 1170 - protein, 14 aa, Homo sapiens
ASPPSQGEVPPGPL

SEQ ID NO: 1171 - protein, 9 aa, synthetic construct
  REGION 1..9: note = Description of Artificial Sequence: Synthetic peptide
FTFSSFAMY

SEQ ID NO: 1172 - protein, 9 aa, synthetic construct
  REGION 1..9: note = Description of Artificial Sequence: Synthetic peptide
FTFGSFSMD

SEQ ID NO: 1173 - protein, 118 aa, synthetic construct
  source 1..118: note = Heavy chain variable domain
EVQLVQSGGVVVQPGGSLRLSCAASGFTFDDYTMHWVRQAPGKGLEWVSLISWDGGSTYYADSVKGRFTISRDNSKNSLYLQMNSLRTEDTALYYCAKDADDSTFDIWGQGTMVTVSS

SEQ ID NO: 1174 - protein, 107 aa, synthetic construct
  source 1..107: note = Light chain variable domain
ETTLTQSPATLSVSPGERVTLSCRASQSVSRNLAWYQQKPGQPPKLLIYWASTRESGVPDRFSGSGSGTDFTLTISSLQAEDVAVYYCQQYYSVPYTFGQGTKLEIK

SEQ ID NO: 1175 - protein, 121 aa, synthetic construct
  source 1..121: note = Heavy chain variable domain
QMQLVQSGAEVKKPGSSVKVSCKASGGTFSSYAISWVRQAPGQGLEWMGGIIPMFGTTNYAQKFQGRVTIIADESTSTAYMELRSLRSDDTAVYYCARDREWEPAYGMDVWGQGTTVTVSS

SEQ ID NO: 1176 - protein, 110 aa, synthetic construct
  source 1..110: note = Light chain variable domain
QSALTQPASVSGSPGQSITISCIGTSSDVGGYNYVSWYQQHPGKAPKLMIYDVSNRPSGVSNRFSGSKSGNTASLTISGLQAEDEAMYYCSAYTVSSTWVFGGGTKVTVL

(19) United States
(12) Patent Application Publication
(10) Pub. No.: US 2026/0250370 A1
(43) Pub. Date: Aug. 27, 2026

(54) TGF-BETA INHIBITORS FOR USE FOR TREATING RESISTANT OR UNRESPONSIVE CANCER IN PATIENTS

(71) Applicant: SCHOLAR ROCK, INC., Cambridge, MA (US)

(72) Inventors: Lu GAN, Cambridge, MA (US); Thomas SCHURPF, Cambridge, MA (US); George CORICOR, Cambridge, MA (US); Justin William JACKSON, Cambridge, MA (US); Si Tuen LEE-HOEFLICH, Cambridge, MA (US); Christopher BRUECKNER, Cambridge, MA (US); Constance MARTIN, Cambridge, MA (US); Ryan FAUCETTE, Cambridge, MA (US); Frederick C. STREICH, Jr., Cambridge, MA (US)

(21) Appl. No.: 19/162,789

(22) PCT Filed: Mar. 7, 2024
(86) PCT No.: PCT/US2024/018970

§ 371 Date: Sep. 5, 2025

Related U.S. Application Data
(60) Provisional application No. 63/488,953, filed on Mar. 7, 2023.
(60) Provisional application No. 63/590,186, filed on Oct. 13, 2023.

(51) Int. Cl. C07K 16/22; A61K 31/337; A61K 31/513; A61K 33/243; A61K 38/14; A61K 39/395; A61P 35/00

(52) U.S. Cl. CPC C07K 16/22; A61K 31/337; A61K 31/513; A61K 33/243; A61K 38/14; A61K 39/3955; A61P 35/00; C07K 2317/21

(57) ABSTRACT

The present disclosure provides TGFβ inhibitor therapy for treating immunosuppressive conditions, such as cancer, either as a monotherapy or combination/adjunct therapy. Selection of suitable therapy and patients who are likely to benefit from such therapy are also disclosed, as well as methods of treating cancer and methods of predicting and monitoring therapeutic response. Related compositions, methods and therapeutic use are also disclosed.

### CROSS-REFERENCE TO RELATED APPLICATIONS

This application claims the benefit of and priority to U.S. Provisional Application No. 63/488,953, filed on Mar. 7, 2023, and U.S. Provisional Application No. 63/590,186, filed on Oct. 13, 2023. The entire contents of each of the aforementioned applications are incorporated herein by reference in their entireties.

### SEQUENCE LISTING

The XML-format Sequence Listing filed herewith, which was created on Feb. 29, 2024 with the name “15094-0056-00304SL.xml” and a size of 555,151 bytes, is incorporated herein by reference in its entirety, FIELD

The instant application relates generally to TGFβ inhibitors and therapeutic use thereof, as well as related assays for diagnosing, monitoring, prognosticating, and treating disorders, including cancer.

### BACKGROUND

Transforming growth factor beta 1 (TGFβ1) is a member of the TGFβ superfamily of growth factors, along with two other structurally related isoforms, namely, TGFβ2 and TGFβ3, each of which is encoded by a separate gene. These TGFβ isoforms function as pleiotropic cytokines that regulate cell proliferation, differentiation, immunomodulation (e.g., adaptive immune response), and other diverse biological processes both in homeostasis and in disease contexts. The three TGFβ isoforms signal through the same cell-surface receptors and trigger similar canonical downstream signal transduction events that include the SMAD2/3 pathway.

Transforming growth factor beta-1 (TGFβ1) has been implicated in mediating immunosuppressive phenotype of tumors, associated with resistance to cancer therapies, such as immune checkpoint inhibitors (Martin et al., Science Translational Medicine, 2020, 12(536): eaay8456).

In immuno-oncology, the phrases “immune dessert” and “immune-excluded” were coined to describe tumors that are devoid of cytotoxic (CD8+) T cells and those that suppress cytotoxic T cells in the tumor microenvironment, respectively, while “inflamed” tumors are that that are infiltrated with cytotoxic T cells.

Applicant previously demonstrated that selectively inhibiting TGFβ1 can be sufficient to overcome tumor immunosuppression by promoting T cell infiltration into tumors, thereby rendering them responsive to checkpoint inhibitors. These results suggested that lack of intratumoral CD8+ T cells may indicate patients who are likely to benefit from TGFβ1 inhibitor therapy (WO/2020/014460, WO 2021/142448 and, WO 2022/256723).

Despite progress, a significant fraction of cancer patients remain resistant or refractory to cancer therapies. There remains unmet need for improved cancer patient selection and treatment.

### SUMMARY

The present disclosure includes the surprising finding that, contrary to the prior belief that having or converting a tumor to an immune-infiltrated status would facilitate the use of treatments such as checkpoint inhibitor therapy, for some patients additional intervention may be needed, e.g., TGFβ1 inhibition may further facilitate treatment, e.g., with checkpoint inhibitor therapy. For example, patients with an elevated level of Tregs in the TME, or who have a higher ratio of Tregs to CD8+ T cells in the TME may benefit from TGFβ1 inhibition despite exhibiting an immune-infiltrated phenotype. Such patients may also exhibit tumors enriched with platelets, wherein optionally the platelets express nicotinamide N-methyltransferase (NNMT). In some embodiments, the tumor may comprise cells undergoing or undergone epithelial-to-mesenchymal transition (EMT), wherein EMT may be optionally characterized by an increased expression of stem-cell-like or mesenchymal markers and/or a reduced expression of epithelial markers. In some embodiments, the stem-cell-like or mesenchymal markers include α-SMA, vimentin, N-cadherin, fibronectin and/or TCF7. These Patients who may benefit from TGFβ1 inhibition may have failed to respond or inadequately responded to prior lines of therapy, e.g., prior checkpoint inhibitor and/or genotoxic therapy. In some patients, an increased level of circulating MDSCs, e.g., gMDSCs, may also be detected. In some embodiments, the carcinoma is renal cell carcinoma, preferably clear cell renal cell carcinoma (ccRCC).

The present disclosure also provides the surprising finding that reactive oxygen species (ROS) can potentiate or otherwise promote TGFβ activation. The TME is characterized by elevated levels of cancer-derived as well as cellular ROS. Furthermore, cancer therapies comprising genotoxic agent such as radiation therapy and chemotherapy, can induce high levels of ROS. Therefore, TGFβ inhibitors, TGFβ1 inhibitors in particular, may provide protective effects in countering ROS-induced damage. Accordingly, a TGFβ inhibitor may be used in the treatment of cancer in a subject who undergoes genotoxic agent therapy, wherein optionally the genotoxic agent therapy is radiation therapy and/or chemotherapy. In preferred embodiments, the TGFβ inhibitor is a TGFβ1-selective inhibitor. Most preferably, the TGFβ1-selective inhibitor is SRK-181.

The present disclosure includes, inter alia, the recognition that lack of intratumoral CD8+ T cells alone is not always a sufficient marker for predicting a patient population likely to benefit from TGFβ1 inhibitor therapy. This recognition is based on the observation that a subset of carcinoma patients (e.g., renal cell carcinoma (RCC) patients) are resistant or refractory to prior cancer therapies, such as checkpoint inhibitor therapies, even though the tumor is infiltrated with cytotoxic T cells. The present disclosure provides, in part, a method of addressing this deficiency in treating immune-infiltrated tumors through the use of TGFβ1 inhibitors to treat a patient, particularly those patients having an immune infiltrated tumor. The TGFβ1 inhibitors may be administered in an amount effective to treat a carcinoma either as monotherapy and/or in conjunction with additional agents. The additional agents may be given in combination with the TGFβ1 inhibitors or as add-on/adjunct therapies, and can include checkpoint inhibitors and/or genotoxic agents, e.g., radiation therapy and chemotherapy. The patient may have received prior cancer therapy, e.g., prior checkpoint inhibitor or genotoxic therapy.

The prior cancer therapies include but are not limited to checkpoint inhibitor therapy, chemotherapy and radiation therapy. In some embodiments, a patient has received multiple lines of prior cancer therapy aimed to treat the carcinoma. In some embodiments, the carcinoma is resistant or unresponsive to the prior cancer therapies. In some embodiments, disease progresses during the prior therapies. In some embodiments, the patients experiences adverse events in response to the prior cancer therapies, leading to the discontinuation of the therapy or therapies.

Examples of prior cancer therapies include but are not limited to: anti-PD-(L)1 (e.g., pembrolizumab, nivolumab, cemiplimab, atezolizumab, dostarlimab, durvalumab, avelumab), anti-CTLA4 (e.g., ipilimumab, tremelimumab), tyrosine kinase inhibitors (e.g., sunitinib, cabozantinib, imatinib, gefitinib, sorafenib, erlotinib, lapatinib, canertinib, semaxinib, vatalanib, leflunomide, etc.), phosphoinositide 3-kinase (PI3K) inhibitors, paclitaxel, carboplatin, topotecan, doxil, gemcitabine, altretamine, bevacizumab, letrozole, carboplatin/taxol, leuprorelin, carboplatin/docetaxel, bevacizumab, etc. In some embodiments, one or more of the listed cancer therapies may be continued in conjunction with the treatments disclosed herein.

In some embodiments, the carcinoma is renal cell carcinoma, preferably clear cell renal cell carcinoma (ccRCC). In some embodiments, the renal cell carcinoma contains tumor-infiltrated CD8+ T cells (e.g., “CD8+ T cell-infiltrated tumor”) but is resistant to or poorly responsive to cancer therapy such as checkpoint inhibitors and genotoxic agents (e.g., radiation therapy and chemotherapy). In some embodiments, the renal cell carcinoma is enriched with Tregs. In some embodiments, the renal cell carcinoma is enriched with platelets, wherein optionally the platelets express nicotinamide N-methyltransferase (NNMT). In some embodiments, the renal cell carcinoma comprises cells undergoing or undergone epithelial-to-mesenchymal transition (EMT), wherein EMT may be optionally characterized by an increased expression of stem-cell-like or mesenchymal markers and/or a reduced expression of epithelial markers. In some embodiments, the stem-cell-like or mesenchymal markers include α-SMA, vimentin, N-cadherin, fibronectin and/or TCF7. In some embodiments, the carcinoma is non-small cell lung carcinoma (NSCLC). In some embodiments, the carcinoma is urothelial carcinoma (UC). In some embodiments, the carcinoma is head and neck carcinoma, such as head and neck squamous cell carcinoma (HNSCC). In some embodiments, the carcinoma is ovarian carcinoma. In some embodiments, the carcinoma is invasive ductal carcinoma of the breast, optionally a triple-negative breast cancer. In some embodiments, the carcinoma is pancreatic adenocarcinoma. In some embodiments, the carcinoma is colorectal carcinoma. In some embodiments, the carcinoma is squamous cell skin carcinoma.

In some embodiments, the patient has a metastasis (i.e., the primary caner has metastasized) at screening (prior to initiating the treatment of the TGFβ1 inhibitor). In some cases, the cancer has metastasized to multiple sites.

The TGFβ1 inhibitor may be administered to the patient in an amount effective to treat the carcinoma. In some embodiments, a therapeutically effective amount is an amount that achieves a stable disease (SD), wherein SD indicates no disease progression for a set period of time, such as 16 weeks or longer (e.g., 6 months, 7 months, 8 months, 9 months, 10 months or longer) upon/during the treatment. In some embodiments, a therapeutically effective amount is an amount that achieves a partial response (PR), e.g., 30% or greater tumor reduction. In some embodiments, tumor reduction is measured by percent change in sum of diameters (SOD) in target lesions from baseline. In some embodiments, the therapeutically effective amount achieves 50% or greater reduction in SOD from baseline. In some embodiments, the response rate, defined as the percentage of patients achieving either a complete response (CR) or PR, is 20% or greater, e.g., 25%, 30%, 35%, 40%, 45%, 50%, or greater. In some embodiments, the durability of response is at least 6 months, e.g., 6 months, 7 months, 8 months, 9 months, 10 months, 11 months, 12 months, or longer.

According to the present disclosure, TGFβ1 inhibitors include any pharmacological agents aimed to reduce and capable of reducing the TGFβ1 signaling pathway. These include, for example: inhibitors of TGFβ1 activators, such as integrins that bind the RGD motif within the LAP domain of latent TGFβ1; inhibitors of TGFβ1 activation, such as antibodies that bind latent TGFβ1 thereby inhibiting the release of the growth factor from the latent complex; inhibitors of the mature (soluble) TGFβ1 ligand, such as neutralizing antibodies, ligand traps that incorporate ligand-binding modules of the TGFβ receptor(s), and nucleic acid-based inhibitors, e.g., siRNA and antisense oligonucleotides; and, TGFβ receptor antagonists, such as Alk5 inhibitors.

In some embodiments, the TGFβ1 inhibitor preferentially inhibits TGFβ1 over TGFβ2 and/or TGFβ3. In some embodiments, the TGFβ1 inhibitor preferentially inhibits TGFβ1 and TGFβ2 over TGFβ3. In preferred embodiments, the TGFβ1 inhibitor is a TGFβ1-selective inhibitor

Non-limiting examples of TGFβ1 inhibitors that may be used in the methods disclosed herein include: SRK-181 (by Scholar Rock), RG6440 (SOF10) (by Roche/Chugai), ABBV-151 (livmoniplimab) (by AbbVie), NIS793 (XOMA-089) (by Novartis), PLN-10195 (by Pliant), ESO14 (by Elpiscience), Cotsiranib (STP705) (by Sirnaomics), Bintrafusp alpha (M7824), Dalutrafusp alpha (AGEN14423), BMS-986416 (AVID200), MK-2225 (by MERCK), PM8001 (by Biotheus), Vactosertib (by Medpacto), BCA101 (by Bicara), TU2218 (NCE401) (by TiUM), ATB-301 (by Autotelic Bio/Clinigen), AdAPT-001 (AIM-001) (by EpicentRx), CART-PSMA-TGF-bRDN (by Tmunity Therapeutics), HCW9218 (by HCW Biologics), SH3051 (by Sanhome), TST005 (by Transcenta), GS19 (GT90008) (by Kintor (Gensun)), BJ-005 (by BJ Bioscience), QLS31901 (by Qilu Pharmaceutical), TQB2858 (by Chia Tal-tianqing), Y101D (by YZY Biopharma), Charis 1000 (C1K) (by Ensol Biosciences), and Fresolimumab (GC1008). In some embodiments, the TGFβ1 inhibitor is SRK-181 (by Scholar Rock), RG6440 (SOF10) (by Roche/Chugai), ABBV-151 (livmoniplimab) (by AbbVie), or Bintrafusp alpha (M7824). In some embodiments, the TGFβ1 inhibitor is a TGFβ1-selective inhibitor such as SRK-181, RG6440 (SOF10) or ABBV-151 (livmoniplimab). In preferred embodiments, the TGFβ1-selective inhibitor is SRK-181.

Additional examples of TGFβ1 inhibitors include antibodies and antigen-binding fragments thereof disclosed in the following publications, as well as those that compete or cross-compete for antigen binding (e.g., sharing overlapping epitopes) with such antibodies: WO 2020/104460, WO 2020/014473, WO 2019/163927, WO 2021/039945, WO 2015/015003, WO 2018/013939, WO 2021/142427, WO 2016/161410, WO 2019/075090, and WO 2020/160291.

In various embodiments, the TGFβ1-selective inhibitor is SRK-181 (also referred to as Ab6 herein) or an antibody or engineered construct comprising antigen-binding fragments (e.g., the 6 CDRs) of Ab6. The CDR sequences of Ab6 are shown in Table 7 and the variable domains are shown in Table 8. In some embodiments, the TGFβ1-selective inhibitor comprises heavy chain CDRs from Ab6 comprising amino acid sequences of SEQ ID NO: 1001 (H-CDR1), SEQ ID NO: 1002 (H-CDR2), SEQ ID NO: 1003 (H-CDR3), and light chain CDRs from Ab6 comprising amino acid sequences of SEQ ID NO: 1004 (L-CDR1), SEQ ID NO: 1005 (L-CDR2), and SEQ ID NO: 1006 (L-CDR3), as defined by the IMTG numbering system. In some embodiments, the TGFβ1-selective inhibitor comprises a heavy chain variable domain from Ab6 comprising SEQ ID NO: 1007 and a light chain variable domain from Ab6 comprising SEQ ID NO: 1008. In some embodiments, the TGFβ1-selective inhibitor comprises a heavy chain from Ab6 comprising SEQ ID NO: 1009 and a light chain from Ab6 comprising SEQ ID NO: 1011. In some embodiments, the TGFβ1-selective inhibitor comprises IgG4 constant domain.

According to the present disclosure, an effective amount of TGFβ1-selective inhibitor such as SRK-181 may be used to treat cancer in patients. For example, SRK-181 is administered to a patient either as monotherapy or combination therapy (e.g., in conjunction with a checkpoint inhibitor) at 240-3000 mg SRK-181 per dose every 2 weeks or 3 weeks, so as to reduce or slow tumor growth. For example, the dosing regimen may be aligned with the dosing schedule for another therapy to be used in a combination therapy, such as a checkpoint inhibitor therapy. For instance, if the TGFβ1 inhibitor is to be used in conjunction with a PD-1 antibody to be dosed every 3 weeks, Q3W dosing schedule can be selected for convenience. Similarly, with a PD-L1 antibody therapy to be dosed every 2 weeks, Q2W dosing schedule can be selected. In some embodiments, an effective amount of the TGFβ1-selective inhibitor, such as SRK-181, is sufficient to achieve stable disease (SD). In some embodiments, an effective amount of the TGFβ1-selective inhibitor, such as SRK-181, is sufficient to achieve partial response (PR).

In some embodiments, the cancer to be treated with the TGFβ1-selective inhibitor, either as monotherapy or combination or adjunct therapy, is characterized by increased alternative end-joining DNA repair or impaired double-strand break repair.

In some embodiments, the cancer to be treated with the TGFβ1-selective inhibitor, either as monotherapy or combination or adjunct therapy, comprises a solid tumor that is resistant or nonresponsive to checkpoint inhibitor therapy, chemotherapy, radiation therapy, or any combinations thereof.

In some embodiments, the cancer to be treated with the TGFβ1-selective inhibitor, either as monotherapy or combination or adjunct therapy is ovarian cancer, renal cell carcinoma, breast cancer (such as triple-negative breast cancer), prostate cancer, or esophagus cancer.

In various embodiments, the cancer to be treated with the TGFβ1-selective inhibitor, either as monotherapy or combination or adjunct therapy, may be carcinoma, wherein optionally the carcinoma is a basal cell carcinoma, squamous cell carcinoma, transitional cell carcinoma, renal cell carcinoma, or adenocarcinoma. In some embodiments, the basal cell carcinoma is basal cell carcinoma of the skin. In some embodiments, the squamous cell carcinoma (SCC) is squamous cell carcinoma of the skin (cutaneous SCC), SCC of the lung, SCC of the esophagus, SCC of the head and neck. In some embodiments, the transitional cell carcinoma is a transitional cell carcinoma of the kidney. In some embodiments, the adenocarcinoma is breast adenocarcinoma, colorectal adenocarcinoma, lung adenocarcinoma, pancreatic adenocarcinoma, or prostate adenocarcinoma.

In various embodiments, the cancer to be treated with the TGFβ1-selective inhibitor, either as monotherapy or combination or adjunct therapy is: uterine corpus endometrial carcinoma (UCEC), thyroid carcinoma (THCA), testicular germ cell tumors (TGCT), skin cutaneous melanoma (SKCM), prostate adenocarcinoma (PRAD), ovarian serous cystadenocarcinoma (OV), lung squamous cell carcinoma (LUSC), lung adenocarcinoma (LUAD), liver hepatocellular carcinoma (LIHC), kidney renal clear cell carcinoma (KIRC), clear cell renal cell carcinoma (ccRCC), head and neck squamous cell carcinoma (HNSCC), glioblastoma multiforme (GMB), esophageal carcinoma (ESCA), colon adenocarcinoma (COAD), breast invasive carcinoma (BRCA), or bladder urothelial carcinoma (BLCA).

In some embodiments, a TGFβ1-selective inhibitor (such as SRK-181) is used in the treatment of cancer in a subject who is or has been treated with a background therapy comprising a checkpoint inhibitor, chemotherapy and/or radiation therapy.

In some embodiments, a genotoxic therapy (such as chemotherapy and/or radiation therapy) is used in the treatment of cancer in a subject, who is treated with a TGFβ1-selective inhibitor (such as SRK-181).

In some embodiments, a TGFβ1-selective inhibitor and a genotoxic therapy are used as combination therapy in the treatment of cancer in a subject, wherein the genotoxic therapy comprises chemotherapy and/or radiation therapy.

In some embodiments, a TGFβ1-selective inhibitor is used as monotherapy in the treatment of cancer in a subject, wherein optionally the TGFβ1-selective inhibitor is SRK-181 (also referred to as Ab6), an antibody that comprises an antigen-binding fragment of Ab6, a variant thereof, or an engineered construct comprising the same. In some embodiments, the subject has a cancer for which no checkpoint inhibitor is approved by a regulatory authority such as the FDA, EMA and MHLW. In some embodiments, the subject has a carcinoma. Optionally, the carcinoma is a basal cell carcinoma, squamous cell carcinoma, transitional cell carcinoma, renal cell carcinoma, adenocarcinoma. In some embodiments, the basal cell carcinoma is basal cell carcinoma of the skin. In some embodiments, the squamous cell carcinoma (SCC) is squamous cell carcinoma of the skin (cutaneous SCC), SCC of the lung, SCC of the esophagus, SCC of the head and neck. In some embodiments, the transitional cell carcinoma is a transitional cell carcinoma of the kidney. In some embodiments, the adenocarcinoma is breast adenocarcinoma, colorectal adenocarcinoma, lung adenocarcinoma, pancreatic adenocarcinoma, or prostate adenocarcinoma. In some embodiments, the subject has ovarian cancer, e.g., ovarian carcinoma.

In various embodiments, a subject or patient to be administered with (e.g., a candidate for) the cancer therapy, genotoxic agent, chemotherapy, radiation therapy, TGFβ inhibitor and/or the TGFβ1-selective inhibitor in accordance with the present disclosure, is naïve to checkpoint inhibitor therapy, chemotherapy and/or radiation therapy.

In various embodiments, a subject or patient to be administered with (e.g., a candidate for) the cancer therapy, genotoxic agent, chemotherapy, radiation therapy, TGFβ inhibitor and/or the TGFβ1-selective inhibitor in accordance with the present disclosure, is a non-responder to a checkpoint inhibitor therapy, chemotherapy and/or radiation therapy.

The cancer therapy, genotoxic agent, chemotherapy, radiation therapy, TGFβ inhibitor and/or the TGFβ1-selective inhibitor is used to treat cancer in the subject who may further receive a checkpoint inhibitor therapy, wherein optionally the checkpoint inhibitor therapy comprises an anti-PD-1 antibody or an anti-PD-L1 antibody.

In some embodiments, the present disclosure also provides i) enhanced methods for image analysis aimed to provide better characterization of the cellular architecture within and surrounding a tumor; ii) improved methods for determining circulatory TGFβ levels aimed to achieve greater accuracy; and/or, iii) LRRC33 as a potential blood-based biomarker indicative of immunosuppression, and/or treatment, e.g., cancer treatment, that incorporates i), ii), and/or iii). Thus, one or more of these features may be employed as part of diagnostic and/or therapeutic regimen for subjects (e.g., patients) either as monotherapy or combination/adjunct therapy to treat cancer.

The present disclosure also relates to compositions comprising TGFβ inhibitors and methods for selecting suitable TGFβ inhibitors for treating certain patient populations, as well as related treatments using the TGFβ inhibitors. The disclosure provides better and more targeted therapeutics and treatment modalities, including improved ways of identifying candidates for treatment and/or monitoring treatment efficacy, e.g., patients or patient populations who are likely to benefit from the TGFβ inhibitor therapy. Related methods, including therapeutic regimens, and methods for manufacturing such inhibitors are encompassed herein. The selection of particular TGFβ inhibitors for therapeutic use is aimed to achieve in vivo efficacy while controlling potential risk, e.g., toxicities known to be associated with pan-inhibition of TGFβ.

The disclosure includes, in some embodiments, methods comprising selecting and/or administering a TGFβ inhibitor that does not target TGFβ3 signaling for therapeutic use. In some embodiments, the TGFβ inhibitor does not inhibit TGFβ2 signaling at a therapeutically effective dose. In some embodiments, the TGFβ inhibitor does not inhibit TGFβ3 signaling at a therapeutically effective dose. In some embodiments, the TGFβ inhibitor does not inhibit TGFβ2 signaling and TGFβ3 signaling at a therapeutically effective dose. In preferred embodiments, such inhibitor is TGFβ1-selective.

Related embodiments include manufacturing methods comprising selecting a TGFβ inhibitor that does not inhibit TGFβ3 and/or TGFβ2 for producing a medicament. In some embodiments, the medicament may be for a cancer therapy. In preferred embodiments, such inhibitor is TGFβ1-selective.

According to the present disclosure, selection of TGFβ inhibitors for therapeutic use may involve testing a candidate TGFβ inhibitor for immune safety. Such tests may include cytokine release assays and may further include platelet assays.

In some embodiments, a candidate TGFβ inhibitor selected to be produced at large scale and used in, e.g., cancer treatment does not trigger cytokine release (described herein) or platelet aggression (described herein). In preferred embodiments, such inhibitor is TGFβ1-selective. In some embodiments, the disclosure provides a method of manufacturing a pharmaceutical composition comprising a TGFβ inhibitor, wherein the method comprises the steps of: i) selecting a TGFβ inhibitor that meets immune safety criteria characterized by: no significant cytokine release triggered as compared to control (such as IgG) in in vitro cytokine release assays and/or in vivo study in which serum concentrations of such cytokines are measured in response to administration of the TGFβ inhibitor; and/or, no significant binding to, aggregation/activation of human platelets, wherein the TGFβ inhibitor is efficacious in one or more preclinical animal models at a dose below MTD or NOAEL as determined in a preclinical toxicology study; ii) producing the TGFβ inhibitor, e.g., an inhibitor selected as described herein, in a culture (e.g., bioreactor) with a volume of 250 L or greater, optionally further comprising: iii) formulating into a pharmaceutical composition comprising the TGFβ inhibitor and an excipient.

In some embodiments, the pharmaceutical composition and/or treatment regimen disclosed herein may further comprise a checkpoint inhibitor (e.g., as a cancer therapy agent, e.g., a PD-1 antibody, a PD-L1 antibody, or a CTLA-4 antibody) either as a separate molecular entity administered separately, as a single formulation (e.g., an admixture), or as part of a single molecular entity, e.g., an engineered multifunctional construct that functions as both a checkpoint inhibitor and a TGFβ inhibitor. In the methods and treatment regimens described herein referring to a cancer therapy agent (e.g., checkpoint inhibitor) and a TGFβ inhibitor, these components may be provided as a single molecular entity.

In various embodiments, the disclosure provided herein involves the use of circulating MDSC levels as a predictive biomarker to improve the diagnosis, monitoring, patient selection, prognosis, and/or continued treatment of a subject being administered a TGFβ inhibitor (e.g., a TGFβ1 inhibitor, e.g., a TGFβ1-selective inhibitor such as Ab6) by monitoring circulating MDSC levels. In some embodiments, the disclosure also encompasses methods of determining therapeutic efficacy and therapeutic agents (e.g., compositions) or regiments for use in subjects with cancer by measuring levels of circulating MDSCs. Without being bound by theory, the instant inventors have discovered that reversal of or overcoming an immunosuppressive phenotype, e.g., in a cancer or related condition that manifests dysregulation of the ECM, by administration of a TGFβ inhibitor can be indicated by analyzing circulating MDSC levels, e.g., in a sample obtained from a subject, e.g., in blood or a blood component, e.g., prior to the time point when a reduction in tumor volume or other biomarkers might be used to confirm treatment efficacy. In some embodiments, circulatory MDSCs are g-MDSCs. In some embodiments, circulatory MDSCs are m-MDSCs. In some embodiments, circulatory MDSCs are g-MDSCs and m-MDSCs. In some embodiments, circulatory MDSCs are characterized by cell-surface expression of LRRC33. The terms circulating and circulatory (as in “circulating MDSCs” and “circulatory MDSCs”) may be used interchangeably.

Tumor-associated MDSC cells may contribute to TGFβ1-mediated immunosuppression in the tumor microenvironment. Previously, Applicant showed that MDSCs were indeed enriched in solid tumors and that inhibition of TGFβ1 in conjunction with a checkpoint inhibitor treatment significantly reduced intratumoral MDSCs, which correlated with slowed tumor growth and, in some cases, achieved complete regression in multiple preclinical tumor models (PCT/US2019/041373). In these efficacy studies, effectiveness of such combination therapy was observed over the course of weeks to months (for example, 6-12 weeks) by monitoring tumor growth. Tumor biopsy may reveal an immune profile of a tumor microenvironment (TME); however, in addition to being invasive, biopsy-based information may be inaccurate or skewed because tumor-infiltrating lymphocytes (TILs) may not be uniformly present within the whole tumor, and therefore, depending on which portion of the tumor is sampled by biopsy, results may vary. To overcome the limitation (e.g., shortcomings) of biopsy-based analyses, data presented herein now establish the correlation between tumor-associated (e.g., intratumoral) MDSC levels and circulatory MDSC levels, raising the possibility that MDSCs measured in blood samples (e.g., whole blood or a blood component, e.g., PBMCs) may serve as a surrogate to more accurately predict patient populations that are likely to benefit from certain therapeutic regimens. MDSCs may be measured in blood samples by flow cytometry. Furthermore, evidence suggests the degree of tumor burden (e.g., the size of tumor) correlates with the relative level of circulating MDSCs in the subject bearing the tumor. Therefore, by monitoring circulating MDSC levels in a subject after receiving the therapy, response to the therapy (e.g., therapeutic effects) may be evaluated without the need for painful biopsies, and sooner than conventional methods. Moreover, more recent findings presented herein identify LRRC33 as a novel cell-surface marker for MDSCs in circulation (e.g., blood samples). This observation raises the possibility that LRRC33 may be used as a blood-based predictive biomarker.

In various embodiments, the instant inventors identify circulating MDSCs, especially gMDSCs, as an early biomarker to predict the efficacy of combination therapy comprising a TGFβ inhibitor. Data disclosed herein show that after TGFβ1 inhibitor treatment, there is a marked reduction in circulating MDSC levels, e.g., as measured in blood or a blood component, which can be detected well before antitumor efficacy outcome can readily be obtained, in some cases shortening the timeline by weeks. Thus, the disclosure provides, the use of circulating MDSCs as a predictive biomarker for the patient's responsiveness to a cancer therapy, e.g., a combination therapy. In related aspects of the disclosure provided herein, the level of circulating MDSC cells may be determined within 1-10 weeks, e.g., 3-6 weeks, following administration of a dose of TGFβ inhibitor, optionally within 3 weeks or at about 3 weeks following administration of the dose of TGFβ inhibitor. In some embodiments, the level of circulating MDSC cells may be determined within 2 weeks following administration of the dose of TGFβ inhibitor. In some embodiments, the level of circulating MDSC cells may be determined at about 10 days following administration of the dose of TGFβ inhibitor.

Cancer immunotherapy may harness or enhance the body's immunity to combat cancer. Without being bound by theory, it is contemplated that low levels of circulating MDSCs in subjects with cancer indicate that the body has retained or restored disease-fighting immunity (e.g., antitumor activity), more specifically, lymphocytes such as CD8+ T cells, which can be mobilized to attack malignant cells. Thus, reduced levels of circulating MDSCs, especially gMDSCs, upon TGFβ inhibitor treatment may indicate pharmacodynamic effects of TGFβ inhibition (e.g., TGFβ1 inhibition) and serve as an early predictive biomarker for therapeutic efficacy when treated with a cancer therapy such as checkpoint inhibitors.

Advantageously, the likelihood of patient's responsiveness to cancer immunotherapy may be assessed by measuring circulating MDSCs, e.g., in blood or a blood component, as an indicator of TGFβ (e.g., TGFβ1)-mediated immunosuppression. In some embodiments, the circulating MDSCs are characterized by expression of one or more of the following markers: CD11b, CD33, CD14, CD15, LOX-1, CD66b, and HLA-DR^{lo/-}. In some embodiments, the circulating MDSCs are G-MDSCs.

Where cancer patients receive a combination therapy comprising a cancer therapy (such as checkpoint inhibitor) and a TGFβ inhibitor that is not selective for TGFβ1 (non-selective TGFβ inhibitor), there may be a greater risk of toxicity. To mitigate or manage such risk, the non-selective TGFβ inhibitor may be administered infrequently or intermittently, for example on an “as-needed” basis. For example, circulating MDSC levels may be monitored periodically in order to determine that the effects of overcoming immunosuppression are sufficiently maintained, so as to ensure antitumor effects of the cancer therapy. During the course of cancer treatment, if MDSCs become elevated, this may indicate that the patient may benefit from additional dose(s) of a TGFβ inhibitor. Such approach may help reduce unnecessary risk and adverse events associated with over-exposure to a TGFβ inhibitor, particularly a non-TGFβ1-selective inhibitor. In some embodiments, the TGFβ inhibitor targets TGFβ1/2 signaling. In some embodiments, the TGFβ inhibitor targets TGFβ1/3 signaling. In some embodiments, the TGFβ inhibitor targets TGFβ1/2/3 signaling. In some embodiments, the TGFβ inhibitor selectively targets TGFβ1 signaling. In some embodiments, a second TGFβ1-selective inhibitor is used to further reduce the frequency of exposure to a non-TGFβ1-selective inhibitor.

In some embodiments, disclosed herein are methods of treating cancer (also described herein in the context of compositions for use in treating cancer or cancer treatments). Also disclosed are methods of predicting, determining, or monitoring therapeutic efficacy in subjects with cancer, e.g., monitoring a patient's responsiveness to treatment and/or making continued treatment decisions based on the monitored parameters. In some embodiments, the cancer is an immune-excluded cancer and/or a myeloproliferative disorder, wherein the myeloproliferative disorder may be myelofibrosis. The cancer may have an immunosuppressive phenotype. In some embodiments, the cancer has an immune-excluded, immunosuppressive phenotype. In some embodiments, the cancer has an immune desert, immunosuppressive phenotype. In certain other embodiments, the cancer is not an immune-excluded or immune desert cancer. In certain embodiments, the cancer is an immune-infiltrated cancer. In certain embodiments, the cancer has an immune-infiltrated, immunosuppressive phenotype. Sometimes, a cancer that has an immune-excluded phenotype comprises <5% CD8+ cells in the tumor and ≥5% CD8+ cells in the margin and/or stroma. Sometimes, a cancer that has an immune desert phenotype comprises <5% CD8+ cells in all tumor compartments. Sometimes, a cancer that has an immune infiltrated phenotype comprises ≥5% CD8+ cells in the tumor. Sometimes, the cancer has an immune-infiltrated phenotype, but the infiltrated CD8+ cells have reduced cytotoxic function, e.g., the CD8+ cells express reduced amounts of cytotoxic enzymes, such as perforin and/or granzyme B. In some embodiments, the cancer is resistant or refractory to a checkpoint inhibitor therapy, such as an anti-PD(L)1 therapy. In some embodiments, the cancer is an immune-excluded cancer and is resistant or refractory to a checkpoint inhibitor therapy, such as an anti-PD(L)1 therapy. Alternatively, in some embodiments, the cancer is an immune-infiltrated cancer and is resistant or refractory to a checkpoint inhibitor therapy, such as an anti-PD(L)1 therapy. In some embodiments, the cancer is a TGFβ1-positive cancer. The TGFβ1-positive cancer may co-express TGFβ1, TGFβ2, and/or TGFβ3. The TGFβ1-positive cancer may be a TGFβ1-dominant tumor. The TGFβ1-positive cancer may be a TGFβ1-dominant tumor and may co-express TGFβ1, TGFβ2, and/or TGFβ3. For instance, the TGFβ1-positive cancer may be a TGFβ1-dominant tumor and may co-express TGFβ1 and TGFβ2. As another example, the TGFβ1-positive cancer may be a TGFβ1-dominant tumor and may co-express TGFβ1 and TGFβ3. Such cancer includes advanced cancer, e.g., metastatic cancer (e.g., metastatic solid tumors) and cancer with a locally advanced tumor (e.g., locally advanced solid tumors). In some embodiments, the treatment comprises administering to the subject a TGFβ inhibitor in an amount sufficient to reduce circulating MDSC levels, especially circulating gMDSC levels. Circulating MDSC levels are reduced as compared to circulating MDSC levels before treatment with the TGFβ inhibitor. In some embodiments, the TGFβ inhibitor is a TGFβ1-selective inhibitor.

In some embodiments, the cancer is a solid tumor, such as an advanced solid tumor, which solid tumor may be resistant or refractory to a checkpoint inhibitor therapy, such as an anti-PD(L)1 therapy. For example, the cancer may be a carcinoma. In some embodiments, the carcinoma comprises cells that have undergone epithelial-to-mesenchymal transition (EMT). The cancer may additionally or alternatively be non-small cell lung cancer (NSCLC), urothelial carcinoma, melanoma, renal cell carcinoma (such as clear cell renal cell carcinoma (ccRCC)), or head and neck cancer. The cancer may be, or may be suspected of being, an immune-excluded cancer, and optionally may also be or may be suspected of having an immunosuppressive phenotype. Sometimes, e.g., when the cancer is ccRCC, the cancer may be, or may be suspected of being, an immune-infiltrated cancer, and optionally may also be or may be suspected of having an immunosuppressive phenotype. When the cancer is a renal cell carcinoma, and especially when the cancer is ccRCC, the cancer may comprise cells that have undergone epithelial-to-mesenchymal transition (EMT).

In some embodiments, the cancer is a solid tumor, such as an advanced solid tumor, which solid tumor may be resistant or refractory to a checkpoint inhibitor therapy, such as an anti-PD(L)1 therapy, and the patient is administered a TGFβ1 inhibitor, such as a TGFβ1-selective inhibitor, in combination with a checkpoint inhibitor therapy, such as a PD-1 antagonist, a PDL1 antagonist, or a CTLA4 antagonist. For example, the cancer may be non-small cell lung cancer (NSCLC), urothelial carcinoma, melanoma, clear cell renal cell carcinoma (ccRCC), or head and neck cancer. The cancer may be, or may be suspected of being, an immune-excluded cancer, and optionally may also be or may be suspected of having an immunosuppressive phenotype. Sometimes, e.g., when the cancer is ccRCC, the patient is administered a TGFβ1 inhibitor, such as a TGFβ1-selective inhibitor, in combination with a PD-1 antagonist, a PDL1 antagonist, or a CTLA4 antagonist. The checkpoint inhibitor therapy may be an anti-PD-1 antibody, an anti-PDL1 antibody, or an anti-CTLA4 antibody. Sometimes, e.g., when the cancer is ccRCC, the patient is administered a TGFβ1 inhibitor, such as a TGFβ1-selective inhibitor, in combination with a CTLA4 antagonist (e.g., anti-CTLA4 antibody).

In some embodiments, the subject has already received at least one prior line of cancer therapy, such as at least two, three, four, or five prior lines of therapy. Prior lines of therapy may include checkpoint inhibitor therapies (such as PD-1 antagonists, PD-L1 antagonists, and/or CTLA4 antagonists), chemotherapy, and/or radiotherapy. In some embodiments, the subject has already received one or more prior lines of therapy, and at least one of the prior lines of therapy is a checkpoint inhibitor therapy (such as a PD-1 antagonist, a PD-L1 antagonist, and/or a CTLA4 antagonist). In some embodiments, the subject has already received at least one, two, three, four, or five prior lines of therapy, and at least one of the prior lines of therapy is a checkpoint inhibitor therapy (such as a PD-1 antagonist, a PD-L1 antagonist, and/or a CTLA4 antagonist). In some embodiments, the subject has received at least two prior lines of therapy. In some embodiments, the subject has received at least three prior lines of therapy. In some embodiments, the subject has received at least four prior lines of therapy.

In some embodiments, the disclosure encompasses a method of predicting or determining therapeutic efficacy in a subject having cancer comprising the steps of determining circulating MDSC levels (e.g., circulating gMDSC levels) in the subject prior to administering a TGFβ inhibitor (alone or in combination with a cancer therapy), administering to the subject a therapeutically effective amount of the TGFβ inhibitor (alone or in combination with a cancer therapy), and determining circulating MDSC levels in the subject after the administration, wherein a reduction in circulating MDSC levels after administration, as compared to circulating MDSC levels before administration, predicts therapeutic efficacy. In an embodiment, circulating MDSC levels are determined by measuring MDSCs in a blood sample by flow cytometry.

In some embodiments, the disclosure encompasses a method of determining therapeutic efficacy of a cancer treatment in a subject, wherein the treatment comprises administering to the subject a combination therapy comprising a dose of a TGFβ inhibitor and a cancer therapy, the method comprising the steps of (i) determining the circulating MDSC level (e.g., the circulating gMDSC level) in a sample obtained from the subject prior to administering the TGFβ inhibitor, (ii) determining the circulating MDSC level in a sample obtained from the subject after administration of the TGFβ inhibitor, and (iii) determining whether the level determined in step (ii) is reduced compared to the level determined in step (i), such reduction being indicative of therapeutic efficacy of the cancer treatment. In some embodiments, the dose of the TGFβ inhibitor and the cancer therapy in the combination therapy are for concurrent (e.g., simultaneous), separate, or sequential administration. In some embodiments, the TGFβ inhibitor is a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ inhibitor is Ab6.

In some embodiments, the disclosure includes a method of treating cancer in a subject, comprising the steps of determining circulating MDSC levels (e.g., circulating gMDSC levels) in the subject prior to administering a TGFβ inhibitor, administering to the subject a first therapeutically effective dose of the TGFβ inhibitor, determining circulating MDSC levels in the subject after administering the TGFβ inhibitor, and administering to the subject a second therapeutically effective dose of the TGFβ inhibitor or combination therapy if the circulating MDSC levels measured after administering the first therapeutically effective dose of the TGFβ inhibitor are reduced as compared to the circulating MDSC levels measured prior to administering the first therapeutically effective dose of the TGFβ1 inhibitor. In some embodiments, a combination therapy comprising a second cancer therapy (e.g., checkpoint inhibitor therapy) is administered concurrently, sequentially, or simultaneously with the first therapeutically effective dose of the TGFβ inhibitor and the combination therapy is continued if the circulating MDSC levels measured after administering the first therapeutically effective dose of the combination therapy are reduced as compared to the circulating MDSC levels measured prior to administering the first therapeutically effective dose.

In some embodiments, the disclosure encompasses a cancer therapy agent for use in the treatment of cancer in a subject, wherein the subject has received a dose of a TGFβ inhibitor and wherein the circulating MDSC level (e.g., the circulating gMDSC level) in the subject measured after administration of the TGFβ inhibitor has been determined to be reduced as compared to the circulating MDSC level measured in the subject prior to administering the dose of the TGFβ inhibitor. In some embodiments, the TGFβ inhibitor is a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ inhibitor is Ab6.

In some embodiments, the disclosure encompasses a combination therapy comprising a dose of a TGFβ inhibitor and a cancer therapy agent for use in the treatment of cancer, wherein the treatment comprises concurrent (e.g., simultaneous), separate, or sequential administration to a subject of a dose of the TGFβ inhibitor and the cancer therapy agent, and wherein the circulating MDSC level (e.g., the circulating gMDSC level) in the subject measured after the administration of the TGFβ inhibitor has been determined to be reduced as compared to the circulating MDSC level measured in the subject prior to administering the dose of the TGFβ inhibitor. In some embodiments, the TGFβ inhibitor is a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ inhibitor is Ab6.

In some embodiments, the disclosure encompasses a TGFβ inhibitor for use in the treatment of cancer in a subject, wherein the subject has received at least a first dose of the TGFβ inhibitor, and wherein the treatment comprises administering a further dose of the TGFβ inhibitor, provided that the circulating MDSC level (e.g., the circulating gMDSC level) in the subject measured after the administration of the at least first dose of the TGFβ inhibitor is reduced as compared to the circulating MDSC level measured in the subject prior to administering a dose of the TGFβ inhibitor. In some embodiments, the TGFβ inhibitor is a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ inhibitor is Ab6.

In some embodiments, the disclosure encompasses a TGFβ inhibitor for use in the treatment of cancer in a subject, wherein the subject is administered a dose of the TGFβ inhibitor, and wherein the TGFβ inhibitor reduces or reverses immune suppression in the cancer, wherein said reduced or reversed immune suppression has been determined by a reduction in the circulating MDSC level (e.g., the circulating gMDSC level) in the subject measured after the administration of the TGFβ inhibitor as compared to the circulating MDSC level measured in the subject prior to administering the dose of the TGFβ inhibitor. In some embodiments, the TGFβ inhibitor is a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ inhibitor is Ab6.

In some embodiments, the disclosure encompasses a method of treating advanced cancer in a human subject comprising the steps of selecting a subject with advanced cancer comprising a locally advanced tumor and/or metastatic cancer with primary resistance to a checkpoint inhibitor therapy, administering a TGFβ inhibitor, and administering to the subject a checkpoint inhibitor therapy. In the methods and compositions for use in cancer treatment described herein, the cancer may be advanced cancer. It may comprise a locally advanced tumor and/or metastatic cancer with primary resistance to a checkpoint inhibitor therapy. The cancer therapy may comprise a checkpoint inhibitor therapy. The subject may be a human subject. In some embodiments, the subject has elevated circulating MDSC levels (e.g., circulating gMDSC levels), e.g., as compared to a healthy control subject or as compared to a control subject with a cancer that is not resistant to (e.g., is responsive to) the checkpoint inhibitor therapy. In some embodiments, a subject has elevated circulating MDSC levels (e.g., circulating gMDSC levels) if circulating MDSCs (e.g., circulating gMDSCs) are detectable in a sample, such as above 1% of the white blood cell component/PBMC component or above 0.1% of whole blood. In some embodiments, a subject has elevated circulating MDSC levels (e.g., circulating gMDSC levels) if circulating MDSCs (e.g., circulating gMDSCs) are above 1%, 2%, 3%, 4%, 5%, 6%, 7%, 8%, 9%, 10%, 15%, 20%, 25%, 30%, 35%, 40%, 45%, 50%, 55%, or 60% of the white blood cell component/PBMC component of a blood sample. In some embodiments, a subject has elevated circulating MDSC levels (e.g., circulating gMDSC levels) if circulating MDSCs (e.g., circulating gMDSCs) are above 10% of the white blood cell component/PBMC component of a blood sample. In some embodiments, treatment reduces the level of circulating MDSCs. In some embodiments, continued treatment is contingent on an observed reduction in circulating MDSCs.

In some embodiments, the disclosure encompasses a method of treating, predicting, determining, and/or monitoring therapeutic efficacy of a cancer treatment in a subject administered a TGFβ inhibitor alone or in combination with another cancer therapy (e.g., checkpoint inhibitor). The method comprises the steps of determining the levels of tumor-associated immune cells (e.g., CD8+ T cells and tumor-associated macrophages) in the subject prior to administering a treatment, administering the treatment to the subject, and determining the levels of tumor-associated immune cells in the subject after administering the treatment, wherein a change in the level of one or more tumor-associated immune cell populations after inhibitor administration, as compared to the levels of tumor-associated immune cells before administration, indicates therapeutic efficacy. In some embodiments, treatment alters the level of tumor-associated immune cells. In some embodiments, continued treatment is contingent on an observed change in tumor-associated immune cells. In some embodiments, the tumor-associated immune cell levels are monitored in combination with monitoring circulating MDSC levels (e.g., circulating gMDSC levels) and treatment efficacy and/or continued treatment is contingent on observed changes in both sets of biomarkers (e.g., a reduction in circulating MDSC levels and an increase in tumor-associated CD8+ T cells).

In some embodiments, the disclosure provides a checkpoint inhibitor and a TGFβ1 inhibitor for use in the treatment of cancer in a subject in need thereof, wherein the treatment comprises administration of a checkpoint inhibitor and a TGFβ1 inhibitor in amounts effective to treat cancer, wherein optionally the checkpoint inhibitor is a PD-(L)1 inhibitor, wherein further optionally the PD-(L)1 inhibitor is budigalimab; wherein optionally the TGFβ1 inhibitor is a TGFβ1-selective inhibitor, wherein further optionally the TGFβ1-selective inhibitor is SRK-181 (also referred to as Ab6 herein); and, wherein optionally the cancer comprises a solid tumor of immunosuppressive phenotype.

In some embodiments, the disclosure provides a checkpoint inhibitor for use in the treatment of cancer in a subject in need thereof, wherein the treatment comprises administration of a checkpoint inhibitor to the subject treated with a TGFβ1 inhibitor, in amounts effective to treat cancer, wherein optionally the checkpoint inhibitor is a PD-(L)1 inhibitor, wherein further optionally the PD-(L)1 inhibitor is budigalimab; wherein optionally the TGFβ1 inhibitor is a TGFβ1-selective inhibitor, wherein further optionally the TGFβ1-selective inhibitor is SRK-181 (also referred to as Ab6 herein); and, wherein optionally the cancer comprises a solid tumor of immunosuppressive phenotype.

In some embodiments, the disclosure provides a TGFβ1 inhibitor for use in the treatment of cancer in a subject in need thereof, wherein the treatment comprises administration of a TGFβ1 inhibitor to the subject treated with a checkpoint inhibitor, in amounts effective to treat cancer, wherein optionally the checkpoint inhibitor is a PD-(L)1 inhibitor, wherein further optionally the PD-(L)1 inhibitor is budigalimab; wherein optionally the TGFβ1 inhibitor is a TGFβ1-selective inhibitor, wherein further optionally the TGFβ1-selective inhibitor is SRK-181 (also referred to as Ab6 herein); and, wherein optionally the cancer comprises a solid tumor of immunosuppressive phenotype.

In some embodiments, the disclosure encompasses methods of treating, predicting, determining, and/or monitoring therapeutic efficacy of a cancer treatment in a subject. In some embodiments, the method comprises measuring levels of CD8+ cells in the tumor (or in one or more tumor nests within the tumor) and the surrounding stroma and/or margin compartments in one or more tumor samples obtained from the subject. In some embodiments, the method comprises identifying the immune phenotype of the subject's cancer based on the level of CD8+ cells inside the tumor or tumor nest(s) as compared to the level of CD8+ cells outside of the tumor or tumor nest(s) (e.g., the surrounding stroma and/or margin compartments). In certain embodiments, the cancer treatment comprises a TGFβ inhibitor, e.g., a TGFβ1 inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, or Ab46. In certain embodiments, the cancer treatment comprises Ab6. In certain embodiments, the cancer treatment comprises an immune checkpoint inhibitor. In certain embodiments, the cancer treatment comprises a TGFβ1 inhibitor (e.g., Ab6) and an immune checkpoint inhibitor (e.g., a PD-1 antibody, a PD-L1 antibody, or a CTLA-4 antibody).

In some embodiments, the disclosure provides a method of treating, predicting, and/or monitoring therapeutic efficacy of a cancer treatment in a subject administered a TGFβ inhibitor alone or in combination with another cancer therapy (e.g., checkpoint inhibitor). The method comprises the steps of determining the levels of circulating latent TGFβ in the subject prior to administering a treatment, administering the treatment to the subject, and determining the levels of circulating latent TGFβ in the subject after administering the treatment, wherein a change (e.g., increase) in circulating latent TGFβ after inhibitor administration, as compared to circulating latent TGFβ before administration, indicates therapeutic efficacy. In some embodiments, treatment alters the level of circulating latent TGFβ. In some embodiments, continued treatment is contingent on an observed change (e.g., increase) in circulating latent TGFβ. In some embodiments, the circulating latent TGFβ is monitored in combination with monitoring circulating MDSC levels (e.g., circulating gMDSC levels) and/or tumor-associated immune cell levels. In some embodiments, treatment efficacy and/or continued treatment is contingent on observed changes in two or more sets of biomarkers. In various embodiments, the methods and compositions disclosed herein for use in treating cancer that involve a determination of circulating MDSC levels (and optionally also the assessment of a change in the level of one or more tumor-associated immune cell populations) may further comprise the assessment of the level of circulating latent TGFβ, as described herein. Also disclosed is a composition comprising a therapeutically effective dose of a TGFβ inhibitor for use in treating cancer, wherein the TGFβ inhibitor is administered if a reduction in circulating MDSC levels are determined (alone or in combination with a change in circulating latent TGFβ) after administration of a previous dose of a TGFβ inhibitor. In some embodiments, the TGFβ inhibitor is a TGFβ1-selective inhibitor, e.g., Ab6. In some embodiments, continued treatment is contingent on an observed change in circulating latent TGFβ. In some embodiments, the circulating latent TGFβ is monitored in combination with monitoring circulating MDSC levels and/or tumor-associated immune cell levels. In some embodiments, treatment efficacy and/or continued treatment is contingent on observed changes in two or more sets of biomarkers (e.g., a reduction in circulating MDSC levels and/or an increase in tumor-associated CD8+ T cells and/or a decrease in circulating latent TGFβ).

In some embodiments, the disclosure provides a method of treating cancer, comprising administering to a subject a TGFβ inhibitor (e.g., a TGFβ1 inhibitor) in a therapeutically effective amount that does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1β), and chemokine C—C motif ligand 2 (CCL2)/monocyte chemoattractant protein 1 (MCP-1). In some embodiments, the method does not induce a significant increase in platelet binding, activation, and/or aggregation. In some embodiments, the cancer has elevated circulating MDSC levels (e.g., circulating gMDSC levels), e.g., as compared to a healthy control subject or as compared to a control subject with a cancer that is not resistant to (e.g., is responsive to) the checkpoint inhibitor therapy. In some embodiments, a subject has elevated circulating MDSC levels (e.g., circulating gMDSC levels) if circulating MDSCs (e.g., circulating gMDSCs) are detectable in a sample, such as above 1% of the white blood cell component/PBMC component, or above 0.1% of whole blood. In some embodiments, a subject has elevated circulating MDSC levels (e.g., circulating gMDSC levels) if circulating MDSCs (e.g., circulating gMDSCs) are above 1%, 2%, 3%, 4%, 5%, 6%, 7%, 8%, 9%, 10%, 15%, 20%, 25%, 30%, 35%, 40%, 45%, 50%, 55%, or 60% of the white blood cell component/PBMC component of a blood sample. In some embodiments, a subject has elevated circulating MDSC levels (e.g., circulating gMDSC levels) if circulating MDSCs (e.g., circulating gMDSCs) are above 10% of the white blood cell component/PBMC component of a blood sample. In some embodiments, treatment with a therapeutically effective amount of the TGFβ inhibitor (e.g., a TGFβ1 inhibitor) reduces the level of circulating MDSCs. In some embodiments, continued treatment is contingent on an observed reduction in circulating MDSCs.

In some embodiments, the disclosure provides a method for identifying whether a TGFβ inhibitor (e.g., a TGFβ1 inhibitor) will be tolerated in a patient, comprising contacting a cell culture or fluid sample with the TGFβ inhibitor and determining whether it causes a significant release of one or more cytokines selected from interferon gamma (IFNγ), interleukin 2 (IL-2), interleukin 6 (IL-6), tumor necrosis factor alpha (TNFα), interleukin 1 beta (IL-1β) and chemokine C—C motif ligand 2 (CCL2)/monocyte chemoattractant protein 1 (MCP-1), wherein a significant release indicates the TGFβ inhibitor will not be well tolerated. The method may comprise monitoring cytokine release in an in vitro cytokine release assay. In some embodiments, the assay is in peripheral blood mononuclear cells (PBMCs) or whole blood, optionally wherein the PBMCs or whole blood are obtained from the subject prior to administering a TGFβ inhibitor therapy. In some embodiments, the disclosure encompasses a TGFβ inhibitor (e.g., a TGFβ1-selective inhibitor) for use in the treatment of cancer by administering to a subject a dose of said TGFβ inhibitor, wherein said TGFβ inhibitor does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1p) and chemokine C—C motif ligand 2 (CCL2)/monocyte chemoattractant protein 1 (MCP-1). In some embodiments, the disclosure encompasses a combination therapy comprising a dose of a TGFβ inhibitor (e.g., a TGFβ1 inhibitor) and a cancer therapy agent (e.g., a checkpoint inhibitor therapy) for use in the treatment of cancer, wherein the treatment comprises simultaneous, concurrent, or sequential administration to a subject of a dose of the TGFβ inhibitor and the cancer therapy agent, wherein said TGFβ inhibitor does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1p) and chemokine C—C motif ligand 2 (CCL2)/monocyte chemoattractant protein 1 (MCP-1). In some embodiments, the TGFβ inhibitor for use in the treatment of cancer is administered in a therapeutically effective amount that is sufficient to reduce circulating MDSCs (e.g., circulating gMDSCs). Circulating MDSC levels are reduced as compared to circulating MDSC levels before treatment with the TGFβ inhibitor, i.e., as compared to baseline circulating MDSC levels.

In some embodiments, the disclosure provides a method for determining whether a TGFβ inhibitor (e.g., a TGFβ1 inhibitor) causes a significant increase in platelet binding, activation and/or aggregation following exposure of the sample to said TGFβ inhibitor, which method comprises measuring platelet binding, activation and/or aggregation in a plasma or whole blood sample. In some embodiments, the disclosure encompasses a TGFβ inhibitor (e.g., a TGFβ1 inhibitor) for use in the treatment of cancer by administering to a subject a dose of said TGFβ inhibitor, wherein said TGFβ inhibitor does not cause a significant increase in platelet binding, activation and/or aggregation. In some embodiments, the disclosure encompasses a combination therapy comprising a dose of a TGFβ inhibitor (e.g., a TGFβ1 inhibitor) and a cancer therapy agent (e.g., a checkpoint inhibitor therapy) for the treatment of cancer, wherein the treatment comprises concurrent (e.g., simultaneous), separate, or sequential administration to a subject of a dose of the TGFβ inhibitor and the cancer therapy agent, wherein said TGFβ inhibitor does not cause a significant increase in platelet binding, activation and/or aggregation. In some embodiments, the TGFβ inhibitor for use is administered in a therapeutically effective amount that is sufficient to reduce circulating MDSCs (e.g., circulating gMDSCs). Circulating MDSC levels are reduced as compared to circulating MDSC levels before treatment with the TGFβ inhibitor, i.e., as compared to baseline circulating MDSC levels.

In various embodiments of the methods and compositions disclosed herein where the subject is evaluated for circulating MDSC levels (e.g., circulating gMDSC levels), the subject may have a cancer, e.g., a highly metastatic cancer. In some embodiments, the subject has melanoma, triple-negative breast cancer, HER2-positive breast cancer colorectal cancer (e.g., microsatellite stable-colorectal cancer, lung cancer (e.g., non-small cell lung cancer or small cell lung cancer), pancreatic cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer (e.g., gastric cancer), or thyroid cancer.

In some embodiments, the disclosure provides a method of making a TGFβ inhibitor for treating cancer in a subject, comprising the steps of selecting a TGFβ inhibitor which satisfies one or more, or e.g., all of, the following criteria: a) the TGFβ inhibitor is efficacious in one or more preclinical models, b) the TGFβ inhibitor does not cause valvulopathies or epithelial hyperplasia in toxicology studies in one or more animal species at a dose at least greater than a minimum efficacious dose, c) the TGFβ inhibitor does not induce significant cytokine release from human PBMCs or whole blood in an in vitro cytokine release assay at the minimum efficacious dose as determined in the one or more preclinical models of (a), d) the TGFβ inhibitor does not induce a significant increase in platelet binding, activation, and/or aggregation at the minimum efficacious dose as determined in the one or more preclinical models of (a), and e) the TGFβ inhibitor reduces circulating MDSCs (e.g., circulating gMDSCs) at the minimum efficacious dose as determined in the one or more preclinical models of (a), wherein the method further comprises manufacturing a pharmaceutical composition comprising the TGFβ inhibitor and a pharmaceutically acceptable excipient. In some embodiments, the selected TGFβ inhibitor is a TGFβ1-selective inhibitor. In some embodiments, the TGFβ inhibitor is selective for pro- and/or latent TGFβ1.

In some embodiments, the methods of the present disclosure may be used to select and treat patients exhibiting resistance to immunotherapy, e.g., to checkpoint inhibitor therapy. The patient or subject referred to in the methods and compositions for use disclosed herein may have resistance to immunotherapy, e.g., checkpoint inhibitor therapy. Patient populations encompassed by the current disclosure may be treatment-naïve (e.g., may have not received previous cancer therapy), have primary resistance (i.e., present before treatment initiation), or have acquired resistance to an immunotherapy, e.g., checkpoint inhibitor therapy.

In some embodiments, the disclosure encompasses a TGFβ1-selective inhibitor for use in the treatment of cancer wherein the treatment comprises the steps of selecting a subject whose cancer is highly metastatic and administering to the subject an isoform-selective TGFβ1 inhibitor. In some embodiments, the highly metastatic cancer comprises melanoma, triple-negative breast cancer, HER2-positive breast cancer, colorectal cancer (e.g., microsatellite stable-colorectal cancer), lung cancer (e.g., non-small cell lung cancer, small cell lung cancer), bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer (e.g., gastric cancer), or thyroid cancer.

In some embodiments, the disclosure encompasses a TGFβ1-selective inhibitor for use in the treatment of cancer in a subject wherein the treatment comprises the steps of selecting a subject having a myelofibrotic disorder, or is at risk of developing a myelofibrotic disorder, and administering to the subject the TGFβ1-selective inhibitor in an amount effective to treat the cancer.

In some embodiments, the disclosure encompasses a method of treating cancer in a subject, wherein the subject has previously, is currently, or will be treated with a TGFβ inhibitor that inhibits TGFβ3, e.g., in conjunction with a checkpoint inhibitor. These patients may have reduced dosage or treatment frequency by monitoring circulating MDSC levels (e.g., circulating gMDSC levels) and only administering treatment when MDSC levels rise. These patients may also have reduced dosage or treatment frequency by adding in one or more doses of a TGFβ1 or TGFβ1/2 inhibitor. In some embodiments, the patient may have been previously treated with a TGFβ inhibitor that inhibits TGFβ3 in conjunction with a checkpoint inhibitor. In some embodiments TGFβ1 or TGFβ1/2 inhibitors for use in treating cancer in a subject are provided, wherein the subject has previously, is currently, or will be treated with a TGFβ inhibitor that inhibits TGFβ3, e.g., in conjunction with a checkpoint inhibitor. In some embodiments, the cancer is a metastatic cancer, a desmoplastic tumor, or myelofibrosis. In some embodiments, the TGFβ inhibitor is a TGFβ1-selective inhibitor, e.g., Ab6 or a variant thereof, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ inhibitor is Ab6. In some embodiments, the TGFβ inhibitor is isoform-non-selective and inhibits TGFβ1/2/3 or TGFβ1/3.

In some embodiments, the disclosure encompasses an isoform-non-selective TGFβ inhibitor for the treatment of cancer comprising the steps of selecting a subject who is not diagnosed with a fibrotic disorder or who is not at high risk of developing a fibrotic disorder, e.g., a subject who does not exhibit elevated MDSC levels as compared to a control sample, and administering to the subject the isoform-non-selective TGFβ inhibitor in an amount effective to treat the cancer. In some embodiments, the isoform-non-selective TGFβ inhibitor is an antibody (or agent) that inhibits TGFβ1/2/3 or TGFβ1/3. In some embodiments, the isoform-non-selective TGFβ inhibitor is an engineered construct comprising a TGFβ receptor ligand-binding moiety.

In some embodiments, the present disclosure encompasses a TGFβ inhibitor for use in an intermittent dosing regimen for cancer immunotherapy in a patient, wherein the intermittent dosing regimen comprises the following steps: measuring circulating MDSCs (e.g., circulating gMDSCs) in a first sample collected from the patient prior to a TGFβ inhibitor treatment; administering a TGFβ inhibitor to the patient treated with a cancer therapy, wherein the cancer therapy is optionally a checkpoint inhibitor therapy; measuring circulating MDSCs in a second sample collected from the patient after the TGFβ inhibitor treatment; continuing with the cancer therapy if the second sample shows reduced levels of circulating MDSCs as compared to the first sample; measuring circulating MDSCs in a third sample; and, administering to the patient an additional dose of a TGFβ inhibitor, if the third sample shows elevated levels of circulating MDSC levels as compared to the second sample. The TGFβ inhibitor is an isoform-non-selective inhibitor. In some embodiments, the isoform-non-selective inhibitor inhibits TGFβ1/2/3, TGFβ1/2 or TGFβ1/3. In some embodiments, the sample is a blood sample or a blood component.

In some embodiments, the present disclosure provides a TGFβ inhibitor for use in the treatment of cancer comprising a solid tumor, e.g., a solid tumor that is a CD8+ T cell-infiltrated tumor, in a patient, wherein the treatment comprises administration of the TGFβ inhibitor in conjunction with a checkpoint inhibitor (CPI) to treat the cancer, wherein the solid tumor has an immune-infiltrated phenotype and is resistant or refractory to a CPI therapy. In some embodiments, the TGFβ inhibitor is a TGFβ1 inhibitor. In some embodiments, the solid tumor is a carcinoma. In some embodiments, the solid tumor is a carcinoma that comprises cells that have undergone epithelial-to-mesenchymal transition (EMT). In some embodiments, the carcinoma is renal cell carcinoma (RCC), especially clear cell renal cell carcinoma (ccCC). In some embodiments, the CPI is a PD-1 antagonist, a PD-L1 antagonist, or a CTLA4 antagonist.

In any of the embodiments discussed herein, the TGFβ inhibitor, such as the TGFβ inhibitor for use in the treatment of cancer, may be a TGFβ1-selective inhibitor, e.g., an anti-TGFβ1 antibody as described herein or having a sequence as disclosed below, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. For example, the TGFβ1-selective inhibitor may be as defined in any of embodiments 1-35 in paragraph [1034] herein. The TGFβ1-selective inhibitor may be an antibody comprising the CDRs and/or the heavy chain variable region (VH) and/or the light chain variable region (VL) of any one of Ab4, Ab5, Ab6, Ab21, Ab22, Ab23, Ab24, Ab25, Ab26, Ab27, Ab28, Ab29, Ab30, Ab31, Ab32, Ab33, and Ab34, or Ab46. For example, the TGFβ1-selective inhibitor may be an antibody comprising the CDRs and/or the VH and/or the VL of Ab6. In preferred embodiments, the TGFβ inhibitor is Ab6.

The TGFβ1-selective inhibitor may be a GARP-selective inhibitor (e.g., a GARP-TGFβ1 complex-selective inhibitor). The TGFβ1-selective inhibitor may be capable of binding to a GARP-TGFβ1 complex and a LRRC33-TGFβ1 complex (and may not be capable of binding to a LTBP1-TGFβ1 complex or a LTBP3-TGFβ1 complex). The TGFβ1-selective inhibitor may alternatively be a context-independent TGFβ1 inhibitor that is capable of binding to the following pro/latent complexes: GARP-TGFβ1, LRRC33-TGFβ1, LTBP1-TGFβ1, and LTBP3-TGFβ1. In some embodiments, the TGFβ1-selective inhibitor is capable of binding to the following pro/latent complexes: GARP-TGFβ1, LRRC33-TGFβ1, LTBP1-TGFβ1, and LTBP3-TGFβ1, and inhibits the release of the mature TGFβ1 growth factor from the pro/latent complexes.

The TGFβ inhibitor may inhibit integrin-dependent activation of TGFβ1. For example, the TGFβ inhibitor may be a TGFβ1-selective inhibitor which inhibits integrin-dependent activation of TGFβ1. The TGFβ inhibitor may be a TGFβ1-selective inhibitor which inhibits integrin-dependent activation of TGFβ1, and which is a context-independent TGFβ1 inhibitor that is capable of binding to the following pro/latent complexes: GARP-TGFβ1, LRRC33-TGFβ1, LTBP1-TGFβ1, and LTBP3-TGFβ1.

Alternatively or in addition, the TGFβ inhibitor may inhibit protease-dependent or protease-induced activation of TGFβ1. For example, the TGFβ inhibitor may be a TGFβ1-selective inhibitor which inhibits protease-dependent or protease-induced activation of TGFβ1. The TGFβ inhibitor may be a TGFβ1-selective inhibitor which inhibits protease-dependent or protease-induced activation of TGFβ1, and which is a context-independent TGFβ1 inhibitor that is capable of binding to the following pro/latent complexes: GARP-TGFβ1, LRRC33-TGFβ1, LTBP1-TGFβ1, and LTBP3-TGFβ1.

In some embodiments, the TGFβ inhibitors disclosed herein are well tolerated in preclinical safety/toxicology studies in doses up to 100, 200, or 300 mg/kg when dosed weekly for at least 4 weeks. Such studies may be carried out in animal models that are known to be sensitive to TGFβ inhibition, such as rats and non-human primates. In some embodiments, the TGFβ inhibitors disclosed herein do not cause observable toxicities associated with pan-inhibition of TGFβ. Observable toxicities may include cardiovascular toxicities (e.g., valvulopathy). Other observable toxicities include epithelial hyperplasia. Yet further observable toxicities are known in the art. In some embodiments, the TGFβ inhibitors disclosed herein do not induce significant cytokine release or platelet aggregation, binding, or activation. The TGFβ inhibitor may not induce significant cytokine release (e.g., as determined by a method described herein). The TGFβ inhibitor may not cause a significant increase in platelet binding, activation and/or aggregation (e.g., as determined by a method described herein). The TGFβ inhibitor may be or may have been determined by a method described herein not to induce significant cytokine release and not to cause a significant increase in platelet binding, activation and/or aggregation.

In some embodiments, the TGFβ inhibitors disclosed herein achieve a sufficient therapeutic window in that effective amounts of the inhibitors shown by in vivo efficacy studies are well below (such as at least 3-fold, at least 6-fold, or at least 10-fold) the amounts or concentrations that cause observable toxicities. In some embodiments, the therapeutically effective amounts of the inhibitors are between about 1 mg/kg and about 30 mg/kg per week. In some embodiments, therapeutically effective amounts of the inhibitors are between about 1 mg/kg and about 10 mg/kg dosed every three weeks. In some embodiments, therapeutically effective amounts of the inhibitors are between about 2 mg/kg and about 7 mg/kg dosed every three weeks.

In some embodiments, the TGFβ inhibitors disclosed herein achieve a sufficient therapeutic window in that effective amounts of the inhibitors shown by in vivo efficacy studies are well below (such as at least 3-fold, at least 6-fold, or at least 10-fold) the amounts or concentrations that cause dose-limiting toxicities (DLTs). DLTs are generally defined by the occurrence of severe toxicities during therapy (e.g., during first cycle of cancer therapy). Such toxicities may be assessed according to the National Cancer Institute's Common Terminology Criteria for Adverse Events (CTCAE) classification, and usually encompass all grade 3 or higher toxicities with the exception of grade 3 nonfebrile neutropenia and alopecia. In some embodiments, DLTs may also include certain a priori untreatable or irreversible grade 2 toxicities (e.g., neurotoxicities, ocular toxicities, or cardiac toxicities), prolonged grade 2 toxicities (e.g., grade 2 toxicities lasting longer than a certain period), and/or the prolongation of the DLT period. Typically, the definition of DLTs exclude toxicities that are clearly related to the disease itself (e.g., disease progression or intercurrent illness). In some embodiments, the therapeutically effective amounts of the inhibitors are between about 1 mg/kg and about 30 mg/kg per week. In some embodiments, therapeutically effective amounts of the inhibitors are between about 1 mg/kg and about 10 mg/kg dosed every three weeks. In some embodiments, therapeutically effective amounts of the inhibitors are between about 2 mg/kg and about 7 mg/kg dosed every three weeks.

In various embodiments, the TGFβ inhibitors disclosed herein (e.g., a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, or Ab46) is used in conjunction with at least one additional therapy. In some embodiments, the at least one additional therapy is a cancer therapy, such as immunotherapy, chemotherapy, radiation therapy (including radiotherapeutic agents), engineered immune cell therapy (e.g., CAR-T therapy), cancer vaccine therapy, and/or oncolytic viral therapy. A cancer therapy may, for example, comprise a cancer therapy agent (e.g., an immunotherapeutic agent, a chemotherapeutic agent, a radiotherapeutic agent, engineered immune cells (e.g., CAR-T cells)), a cancer vaccine and/or a therapeutic oncolytic virus (including any combination thereof). In some embodiments, the cancer therapy is immunotherapy comprising checkpoint inhibitor therapy. The checkpoint inhibitor may comprise an agent targeting programmed cell death protein 1 (PD-1) or programmed cell death protein 1 ligand (PD-L1). For instance, the checkpoint inhibitor may comprise an anti-PD-1 or anti-PD-L1 antibody. In some embodiments, the TGFβ inhibitors disclosed herein (e.g., a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, or Ab46) may be used in conjunction with at least one additional therapy selected from: a PD-1 antagonist (e.g., a PD-1 antibody), a PDL1 antagonist (e.g., a PDL1 antibody), a PD-L1 or PDL2 fusion protein, a CTLA4 antagonist (e.g., a CTLA4 antibody), a GITR agonist e.g., a GITR antibody), an anti-ICOS antibody, an anti-ICOSL antibody, an anti-B7H3 antibody, an anti-B7H4 antibody, an anti-TIM3 antibody, an anti-LAG3 antibody, an anti-OX40 antibody (OX40 agonist), an anti-CD27 antibody, an anti-CD70 antibody, an anti-CD47 antibody, an anti-41BB antibody, an anti-PD-1 antibody, an anti-CD20 antibody, an anti-CD3 antibody, an anti-PD-1/anti-PDL1 bispecific or multispecific antibody, an anti-CD3/anti-CD20 bispecific or multispecific antibody, an anti-HER2 antibody, an anti-CD79b antibody, an anti-CD47 antibody, an antibody that binds T cell immunoglobulin and ITIM domain protein (TIGIT), an anti-ST2 antibody, an anti-beta7 integrin (e.g., an anti-alpha4-beta7 integrin and/or alphaE beta7 integrin), a CDK inhibitor, an oncolytic virus, an indoleamine 2,3-dioxygenase (IDO) inhibitor, and/or a PARP inhibitor.

In the methods and compositions, e.g., compositions for use according to the present disclosure, including those referring to the determination of circulating MDSC levels (e.g., circulating gMDSC levels) following administration of a TGFβ inhibitor (e.g., a TGFβ1-selective inhibitor or an isotype-non-selective TGFβ inhibitor), the subject may not have received previous cancer therapy, e.g., may be treatment-naïve, may have received previous cancer therapy, or may be receiving cancer therapy. A previous cancer therapy may be or be part of the same cancer therapy to be administered according to the invention. The cancer therapy may be checkpoint inhibitor (CPI) therapy. The cancer may be advanced cancer. The cancer may comprise a locally advanced tumor and/or metastatic cancer. Furthermore, the subject may have cancer which exhibits or is suspected of exhibiting immune suppression (e.g., a tumor with an immune-excluded and/or immunosuppressive phenotype). Alternatively, the subject may have a cancer that does not exhibit or is not suspected of exhibiting immune exclusion, such as a cancer that exhibits or is suspected of exhibiting an immune infiltrated phenotype. For instance, the cancer which exhibits or is suspected of exhibiting an immune excluded phenotype may be ccRCC, NSCLC, melanoma, urothelial carcinoma, or head and neck cancer. The cancer which exhibits or is suspected of exhibiting an immune infiltrated phenotype may be ccRCC. The subject who receives or has received the TGFβ inhibitor may have a cancer with a high response rate to checkpoint inhibitor therapy (e.g., overall response rate of greater than 30%, greater 40%, greater than 50%, or greater) and may be resistant to checkpoint inhibitor therapy. Examples of cancer with high response rates to checkpoint inhibitor therapy include, but are not limited to, microsatellite instability-colorectal cancer (MSI-CRC), renal cell carcinoma (RCC), melanoma (e.g., metastatic melanoma), Hodgkin's lymphoma, NSCLC, cancer with high microsatellite instability (MSI-H), cancer with mismatch repair deficiency (dMMR), primary mediastinal large B-cell lymphoma (PMBCL), and Merkel cell carcinoma (e.g., as reported in Haslam et al., JAMA Network Open. 2019; 2(5): e192535). In some embodiments, the subject may have cancer with a low response rate to checkpoint inhibitor therapy (e.g., overall response rate of 30% or less, 20% or less, or 10%, or less) and may be treatment-naïve. In some embodiments, the subject may have cancer with low response rates to checkpoint inhibitor therapy (e.g., overall response rate of 30% or less, 20% or less, or 10%, or less) and may be resistant to checkpoint inhibitor therapy. Examples of cancer with low response rates to checkpoint inhibitor therapy include, but are not limited to, ovarian cancer, gastric cancer, and triple-negative breast cancer.

In some embodiments, a TGFβ inhibitor (e.g., a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, or Ab46) of the present disclosure may be used to improve rates or ratios of complete verses partial responses among the responders of a cancer therapy. Typically, even in cancer types where response rates to a cancer therapy (e.g., a checkpoint inhibitor therapy) are relatively high (e.g., ≥30% responders), complete response rates are low. The TGFβ inhibitors of the present disclosure may therefore be used to increase the fraction of complete responders within the responder population. In preferred embodiments, the TGFβ inhibitor is Ab6.

In some embodiments, the TGFβ inhibitor does not inhibit TGFβ2 signaling at a therapeutically effective dose. In some embodiments, the TGFβ inhibitor does not inhibit TGFβ3 signaling at a therapeutically effective dose. In some embodiments, the TGFβ inhibitor does not inhibit TGFβ2 signaling and TGFβ3 signaling at a therapeutically effective dose. In some embodiments, a TGFβ inhibitor is a TGFβ1-selective inhibitor, e.g., Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46. In preferred embodiments, the TGFβ1-selective inhibitor is Ab6.

The disclosure provides a method of treating fibrosis in a subject, the method comprising steps of administering a therapeutically effective amount of a TGFβ inhibitor to the subject as a loading dose/maintenance dose regimen, wherein the TGFβ inhibitor inhibits TGFβ1 but does not inhibit one or both of TGFβ2 and/or TGFβ3, thereby treating fibrosis in the subject.

According to another aspect, the disclosure provides a method of preventing fibrosis in a subject at risk of developing fibrosis, the method comprising the steps of administering a therapeutically effective amount of a TGFβ inhibitor to the subject as a loading dose/maintenance dose regimen, wherein the TGFβ inhibitor inhibits TGFβ1 but does not inhibit one or both of TGFβ2 and/or TGFβ3, thereby preventing fibrosis in the subject at risk of developing fibrosis. According to some embodiments of the above aspects and embodiments, the method further comprises the steps of: (i) determining a level of collagen, a level of new collagen synthesis, and/or a level of phosphorylated Smad2, present in a fibrotic tissue in the subject prior to administering the TGFβ inhibitor; and (ii) determining a level of collagen, a level of new collagen synthesis, and/or a level of phosphorylated Smad2, present in a fibrotic tissue in the subject after administering the TGFβ inhibitor, wherein a decrease in the level of collagen, the level of new collagen synthesis and/or the level of phosphorylated Smad2 present in the fibrotic tissue in the subject after administration, as compared to prior to administration, indicates therapeutic efficacy.

According to another aspect, the disclosure provides a method of treating fibrosis in a subject, the method comprising steps of administering to the subject a TGFβ inhibitor, wherein the TGFβ inhibitor inhibits TGFβ1 but does not inhibit one or both of TGFβ2 and/or TGFβ3, in an amount effective to reduce the amount of collagen present in a fibrotic tissue in the subject after administration, as compared to the amount of collagen present in the fibrotic tissue in the subject prior to administration; reduce the amount of new collagen synthesis in a fibrotic tissue in the subject after administration, as compared to the amount of new collagen synthesis present in the fibrotic tissue in the subject prior to administration; and/or reduce the amount of phosphorylated Smad2 in a fibrotic tissue in the subject after administration, as compared to the amount of phosphorylated Smad2 present in the fibrotic tissue in the subject prior to administration; thereby treating fibrosis in the subject. According to one embodiment, the method further comprises the steps of (a) determining a level of collagen, a level of new collagen synthesis, and/or a level of phosphorylated Smad2, present in the fibrotic tissue in the subject prior to administering the TGFβ inhibitor; and (b) determining a level of collagen, a level of new collagen synthesis, and/or a level of phosphorylated Smad2, present in the fibrotic tissue in the subject after administering the TGFβ inhibitor. According to some embodiments of the above aspects and embodiments, reduction in the amount of collagen present in the fibrotic tissue, reduction in the amount of new collagen synthesis, and/or reduction in the amount of phosphorylated Smad2 in the fibrotic tissue is determined 24 hours, 48 hours, 72 hours, or 96 hours after administration of the TGFβ inhibitor. According to some embodiments of the above aspects and embodiments, the method further comprises a step of selecting a subject who would benefit from a reduction in a level of collagen, a level of new collagen synthesis, and/or a level of phosphorylated Smad2 in a fibrotic tissue. According to some embodiments of the above aspects and embodiments, the TGFβ inhibitor is administered as a single dose regimen, or as a loading dose/maintenance dose regimen. Phosphorylated Smad2, present in the fibrotic tissue in the subject after administering the TGFβ inhibitor. According to some embodiments, the single dose regimen comprises administration of a single dosage of between about 1 mg/kg to about 100 mg/kg of the TGFβ inhibitor. According to some embodiments, the single dosage is about 3 mg/kg, about 10 mg/kg, or about 30 mg/kg. According to some embodiments of the above aspects and embodiments, the single dosage is administered to the subject weekly, biweekly, or monthly. According to some embodiments of the above aspects and embodiments, the loading dose/maintenance dose regimen comprises a loading dosage of between about 30 mg/kg and about 90 mg/kg and a maintenance dosage of between about 10 mg/kg and about 30 mg/kg. According to some embodiments of the above aspects and embodiments, the loading dosage is about 30 mg/kg and the maintenance dosage is about 10 mg/kg. According to some embodiments of the above aspects and embodiments, the loading dosage is about 90 mg/kg and the maintenance dosage is about 30 mg/kg. According to some embodiments of the above aspects and embodiments, the loading dosage is administered intravenously, and wherein the maintenance dosage is administered subcutaneously. According to some embodiments of the above aspects and embodiments, the loading dosage is administered once, and the maintenance dosage is administered weekly, biweekly, or monthly thereafter. According to some embodiments of the above aspects and embodiments, the fibrosis is pulmonary fibrosis or kidney fibrosis. According to some embodiments, the pulmonary fibrosis is idiopathic pulmonary fibrosis (IPF). According to some embodiments of the above aspects and embodiments, the administration is effective to reduce symptoms of fibrosis in the subject. According to some embodiments, the symptoms of fibrosis are one or more of pulmonary hypertension, right-sided heart failure, respiratory failure, hypoxia, cough, formation of blood clots, pneumonia, and/or lung cancer in the subject. According to some embodiments of the above aspects and embodiments, the subject has been diagnosed with a pulmonary disease. According to some embodiments, the pulmonary disease is an autoimmune disorder of the lung, a viral infection of the lung, or a bacterial infection of the lung. According to some embodiments of the above aspects and embodiments, the subject has received radiation therapy. According to some embodiments, the radiation therapy is for lung cancer.

According to some embodiments of the above aspects and embodiments, the subject has one or more risk factors for fibrosis selected from the group consisting of cigarette smoking, environmental factors and genetic predisposition for lung fibrosis. According to some embodiments of the above aspects and embodiments, the method further comprises a step of selecting a TGFβ inhibitor that inhibits TGFβ1 but does not inhibit one or both of TGFβ2 and/or TGFβ3.

The present disclosure includes selection of subjects or patients who are likely to respond to or benefit from a TGFβ1 inhibition therapy. Related diagnostic methods, as well as methods for monitoring or determining therapeutic response to the TGFβ1 inhibition therapy, are encompassed herein.

Processes and methods for identifying or selecting TGFβ1-selective inhibitors suitable for therapeutic use are encompassed by the disclosure. In preferred embodiments, selection includes one or more antibodies or antigen-binding fragments with particularly advantageous kinetics criteria characterized by: i) sub-nanomolar affinities to each of human LTBP1/3-proTGFβ1 complexes (e.g., K_{D}<1 nM), and, ii) low dissociation rates (k_{OFF}), e.g., ≤5.00E-4, as measured by a suitable in vitro binding/kinetics assay, such as by surface plasmon resonance (SPR), e.g., BIACORE®-based systems. The selected antibody or the plurality of antibodies are evaluated in preclinical studies comprising an efficacy study and a toxicology/safety study, employing suitable preclinical models. Effective amounts of the antibody or the antibodies determined in the efficacy study are below the level that results in undesirable toxicities determined in the toxicology/safety study. Preferably, the antibody or antibodies are selected which has/have at least 3-fold, 6-fold, and more preferably 10-fold therapeutic window. Effective amounts of the antibodies according to the present disclosure may be between about 0.1 mg/kg and about 30 mg/kg when administered weekly. In preferred embodiments, the maximally tolerated dose (MTD) of the antibodies according to the present disclosure is >100 mg/kg when dosed weekly for at least 4 weeks.

### BRIEF DESCRIPTION OF THE FIGURES

FIG. 1 shows a schematic of an exemplary pathology analysis of tumor tissue sample.

FIG. 2 shows a schematic of an exemplary pathology analysis of tumor tissue sample.

FIG. 3A shows a P-Smad2 IHC analysis of melanoma samples.

FIG. 3B shows pSmad-2 signaling in MBT2 tumors following treat with Ab6-mIgG1.

FIG. 4A shows circulating gMDSC and mMDSC levels in whole blood of mice bearing MBT2 tumors.

FIG. 4B shows intratumoral gMDSC and mMDSC levels in mice bearing MBT2 tumors.

FIG. 5 demonstrates mean pharmacokinetic (PK) profiles of SRK-181 by dose.

FIG. 6 depicts the preliminary efficacy by duration of treatment.

FIG. 7 depicts the best response in target lesions in Part A1 and Part A2.

FIGS. 8A-C show exemplary analysis of MDSC by signal filtering.

FIGS. 9A-C shows identification of tumor MDSC populations in various solid cancer samples.

FIGS. 10A-C shows analysis of gMDSC and mMDSC populations in various solid cancer samples.

FIG. 11A depicts LTBP1, LRRC33, and COL3A1 relative gene expression in an adenine model, and FIG. 11B depicts COL3A1 relative gene expression at 24, 48, and 96 hours. FIG. 11C depicts LRRC33 relative gene expression at 24, 48 and 96 hours.

FIG. 12 provides updates on dosage, cancer types and treatment duration from part A of the DRAGON trial (left panel). The right panel provides a summary of treatment responses in 3 ovarian cancer patients who achieved SD for 6 months or longer in response to SRK-181 monotherapy.

FIG. 13 provides a summary of ccRCC patients who achieved PR in response to a combination treatment of SRK-181 and an anti-PD-1. Two sets of images represent pre- and post-treatment collected from patient 1 and patient 2.

FIGS. 14A-E provide images from representative pre- and post-treatment paired biopsies from UC (FIG. 14A), melanoma (FIG. 146B), NSCLC (FIGS. 14C and 14D), and ccRCC (FIG. 14E) patients. The biopsies are CD8-stained to show CD8+ cells within the tumor compartment. FIGS. 14A, B, C, and D show an increase in tumoral CD8+ T cells after treatment with SRK-181 and anti-PD1. FIG. 14E also shows a slight increase in CD8+ infiltration.

FIGS. 15A-E show the primary compartmental analysis of % CD8+ T cells per tumor compartment, for each of the UC (FIG. 15A), melanoma (FIG. 15B), NSCLC (FIGS. 15C and 15D), and ccRCC (FIG. 15E) patients. The corresponding paired biopsies are shown in FIGS. 14A-E. FIGS. 15A, B, C, and D show an increase in tumoral CD8+ T cells after treatment with SRK-181 and anti-PD1.

FIGS. 16A-E show the tumor nest analysis for each of the UC (FIG. 16A), melanoma (FIG. 16B), NSCLC (FIGS. 16C and 16D), and ccRCC (FIG. 16E) patients. The corresponding paired biopsies are shown in FIGS. 14A-E. FIGS. 16A, B, C, and D show the % CD8+ cells plotted against the size of each tumor nest. FIG. 16E shows the % Tumor (by number of cells) showing each phenotype (i.e., excluded, desert, and infiltrated) pre- and post-treatment (% Tumor=nest cell #/total tumor compartment cell #×100). FIGS. 16A, B, C, and D show an increase in infiltrated tumor nests after treatment with SRK-181 and anti-PD1. FIG. 16E also shows a slight increase in CD8+ infiltration.

FIG. 17 is a table showing the correlation between CD8+ T cell infiltration and tumor shrinkage that was observed in some patients. In the table, “++” indicates a large increase, “+” indicates an increase, “+−” indicates no change, “−” indicates a decrease, and “−−” indicates a large decrease.

FIG. 18 is a graph showing baseline CD8+ cell levels in 11 ccRCC patients before treatment with SRK-181. 8 of the patients were found to have tumors that had an infiltrated phenotype, despite the tumors being non-responsive to anti-PD(L)1 therapy.

FIGS. 19A and 19B provide two graphs showing the changes in circulating gMDSC levels after treatment with SRK-181. FIG. 19A shows the mean change in all patients, stratified according to response (PR=partial response, SD=stable disease, PD=progressive disease). FIG. 19B shows the change in circulating gMDSC levels for individual patients with PR. For patients with PR or SD responses, a decrease in circulatory gMDSCs can be seen.

FIG. 20 is another graph showing the changes in circulating gMDSC levels after treatment with SRK-181 in ccRCC patients. FIG. 20 shows the mean change in all ccRCC patients, stratified according to response (PR, SD, PD).

FIGS. 21A-C are an overview of ccRCC patient responses to SRK-181 and anti-PD1 therapy. Provided is: a graph showing duration of treatment (FIG. 21A), highlighting patients with PR, SD, or SD responses; a waterfall graph showing the best response in target lesions, shown as % change from baseline (FIG. 21B); and a spider graph showing changes in tumor volume over time, shown as % change from baseline (FIG. 21C).

FIG. 22 shows data from a CAGA reporter assay, demonstrating that TGFβ1-selective antibodies could block ascorbic acid/Fe(III)Cl-mediated growth factor release from TGFβ C4S Small Latent Complex (SLC). The TGFβ1-selective antibodies tested include Ab46 (Ab 37021), SKR-181 (Ab 36993), Ab42 (Ab 49247), and Ab 36956. The assay were repeated twice and the corresponding data are shown in the two bar graphs respectively. TGFβ1: TGFβ1 growth factor; 1D11: a pan-inhibitor of TGFβ, serving as positive control; Asc: treatment with ascorbic acid+Fe(III)Cl+ EDTA; HuNEG: a human IgG, serving as negative control.

FIGS. 23A and 23B show kallikerein digestion of Latency-Associated Peptide (LAP) in TGFβ1 C4S Small Latent Complex (SLC) with or without pre-incubation with a TGFβ-selective antibody. LAP can be cleaved by kallikrein into a R⁵⁸ LAP-D fragment and a L⁵⁹ LAP-D fragment as shown in FIG. 23A. FIG. 23B shows that kallikerein cleavage of LAP could not be block by any of the TGFβ1-selective antibodies tested, including Ab46 (Ab 37021), SKR-181 (Ab 36993), Ab42 (Ab 49247), and Ab 36956. M: marker; C4S: TGFβ1 C4S SLC; HuNEG: a human IgG, serving as negative control.

### DETAILED DESCRIPTION OF CERTAIN EMBODIMENTS

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

Advanced cancer, advanced malignancy: The term “advanced cancer” or “advanced malignancy” as used herein has the meaning understood in the pertinent art, e.g., as understood by oncologists in the context of diagnosing or treating subjects/patients with cancer. Advanced malignancy with a solid tumor can be locally advanced or metastatic. The term “locally advanced cancer” is used to describe a cancer (e.g., tumor) that has grown outside the organ it started in but has not yet spread to distant parts of the body. Thus, the term includes cancer that has spread from where it started to nearby tissue or lymph nodes. By contrast, “metastatic cancer” is a cancer that has spread from the part of the body where it started (the primary site) to other parts (e.g., distant parts) of the body.

Affinity: Affinity is the strength of binding of a molecule (such as an antibody) to its ligand (such as an antigen). It is typically measured and reported by the equilibrium dissociation constant (K_{D}). In the context of antibody-antigen interactions, K_{D} is the ratio of the antibody dissociation rate (“off rate” or K_{off} or K_{dis}), how quickly it dissociates from its antigen, to the antibody association rate (“on rate” or Kₒₙ) of the antibody, how quickly it binds to its antigen. For example, an antibody with an affinity of ≤5 nM has a K_{D} value that is 5 nM or lower (i.e., 5 nM or higher affinity) determined by a suitable in vitro binding assay. Suitable in vitro assays can be used to measure K_{D} values of an antibody for its antigen, such as Biolayer Interferometry (BLI) and Solution Equilibrium Titration (e.g., MSD-SET). In a preferred embodiment, affinity is measured by surface plasmon resonance (e.g., Biacore®). An antibody with a suitable affinity in a surface plasmon resonance assay may have, e.g., a K_{D} of at most about 1 nM, e.g., at most about 0.5 nM, e.g., at most about 0.5, 0.4, 0.3, 0.2, 0.15 nM, or less.

Antibody: The term “antibody” encompasses any naturally-occurring, recombinant, modified or engineered immunoglobulin or immunoglobulin-like structure or antigen-binding fragment or portion thereof, or derivative thereof, as further described elsewhere herein. Thus, the term refers to an immunoglobulin molecule that specifically binds to a target antigen, and includes, for instance, chimeric, humanized, fully human, and multispecific antibodies (including bispecific antibodies). An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen binding portions thereof, can be produced in hybridomas, by recombinant DNA techniques, or by enzymatic or chemical cleavage of intact antibodies. The term antibodies, as used herein, includes monoclonal antibodies, multispecific antibodies such as bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), respectively. In some embodiments, the term also encompasses peptibodies.

Antigen: The term “antigen” broadly includes any molecules comprising an antigenic determinant within a binding region(s) to which an antibody or a fragment specifically binds. An antigen can be a single-unit molecule (such as a protein monomer or a fragment) or a complex comprised of multiple components. An antigen provides an epitope, e.g., a molecule or a portion of a molecule, or a complex of molecules or portions of molecules, capable of being bound by a selective binding agent, such as an antigen binding protein (including, e.g., an antibody). Thus, a selective binding agent may specifically bind to an antigen that is formed by two or more components in a complex. In some embodiments, the antigen is capable of being used in an animal to produce antibodies capable of binding to that antigen. An antigen can possess one or more epitopes that are capable of interacting with different antigen binding proteins, e.g., antibodies. In the context of the present disclosure, a suitable antigen is a complex (e.g., multimeric complex comprised of multiple components in association) containing a proTGF dimer in association with a presenting molecule. Each monomer of the proTGF dimer comprises a prodomain and a growth factor domain, separated by a furin cleavage sequence. Two such monomers form the proTGF dimer complex. This in turn is covalently associated with a presenting molecule via disulfide bonds, which involve a cysteine residue present near the N-terminus of each of the proTGF monomer. This multi-complex formed by a proTGF dimer bound to a presenting molecule is generally referred to as a large latent complex. An antigen complex suitable for screening antibodies or antigen-binding fragments, for example, includes a presenting molecule component of a large latent complex. Such presenting molecule component may be a full-length presenting molecule or a fragment(s) thereof. Minimum required portions of the presenting molecule typically contain at least 50 amino acids, but more preferably at least 100 amino acids of the presenting molecule polypeptide, which comprises two cysteine residues capable of forming covalent bonds with the proTGFβ1 dimer.

Antigen-binding portion/fragment: The terms “antigen-binding portion” or “antigen-binding fragment” of an antibody, as used herein, refers to one or more fragments of an antibody that retain the ability to specifically bind to an antigen (e.g., TGFβ1). Antigen binding portions include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments, a monovalent fragment consisting of the VL, VH, CL and CH1 domains; (ii) F(ab′)2 fragments, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv (scFv) molecules (see, e.g., Bird et al., (1988) Science 242:423-426; and Huston et al., (1988) Proc. Nat'l. Acad. Sci. USA 85:5879-5883); (vi) dAb fragments (see, e.g., Ward et al., (1989) Nature 341: 544-546); and (vii) minimal recognition units consisting of the amino acid residues that mimic the hypervariable region of an antibody (e.g., an isolated complementarity determining region (CDR)). Other forms of single chain antibodies, such as diabodies are also encompassed. The term antigen binding portion of an antibody includes a “single chain Fab fragment” otherwise known as an “scFab,” comprising an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids.

Bias: In the context of the present disclosure, the term “bias” (as in “biased binding”) refers to skewed or uneven affinity towards or against a subset of antigens to which an antibody is capable of specifically binding. For example, an antibody is said to have bias when the affinity for one antigen complex and the affinity for another antigen complex are not equivalent (e.g., more than five-fold difference in affinity). Context-independent antibodies according to the present disclosure have equivalent affinities towards such antigen complexes (i.e., unbiased or uniform). Preferred biased antibodies of the present disclosure include “matrix-biased” (or “LTBP-biased”) antibodies, which preferentially bind EMC-associated complexes (LTBP1-proTGFβ1 and LTBP3-proTGFβ), such that relative affinities between at least one of the matrix-associated complexes and at least one of the cell-associated complexes (GARP-proTGFβ1 and/or LRRC33-proTGFβ1 complexes) is greater than five-fold. By comparison, antibodies characterized as “unbiased” have approximately equivalent affinities towards such antigen complexes (e.g., less than five-fold difference in affinity).

Binding region: As used herein, a “binding region” is a portion of an antigen (e.g., an antigen complex) that, when bound to an antibody or a fragment thereof, can form an interface of the antibody-antigen interaction. Upon antibody binding, a binding region becomes protected from surface exposure, which can be detected by suitable techniques, such as HDX-MS. Antibody-antigen interaction may be mediated via multiple (e.g., two or more) binding regions. A binding region can comprise an antigenic determinant, or epitope.

Biolayer Interferometry (BLI): BLI is a label-free technology for optically measuring biomolecular interactions, e.g., between a ligand immobilized on the biosensor tip surface and an analyte in solution. BLI provides the ability to monitor binding specificity, rates of association and dissociation, or concentration, with precision and accuracy. BLI platform instruments are commercially available, for example, from ForteBio and are commonly referred to as the Octet® System.

Cancer: The term “cancer” as used herein refers to the physiological condition in multicellular eukaryotes that is typically characterized by unregulated cell proliferation and malignancy. The term broadly encompasses, solid and liquid malignancies, including tumors, blood cancers (e.g., leukemias, lymphomas and myelomas), as well as myelofibrosis.

Cancer-associated fibroblast (CAF): The term “cancer-associated fibroblast (CAF)” used herein, also known as tumour-associated fibroblast, carcinogenic-associated fibroblast, and activated fibroblast, refers to a cell type within the tumor microenvironment that promotes tumorigenic features by initiating the remodelling of the extracellular matrix or by secreting cytokines. CAFs express smooth muscle actin alpha (actin alpha), platelet-derived growth factor receptor alpha (PDGFRα/CD140a), platelet-derived growth factor receptor beta (PDGFRβ/CD140b), fibroblast specific protein 1 (FSP-1/S100A4), fibroblast activation protein (FAP), and nicotinamide N-methyltransferase (NNMT), all of which have been used as markers to identify CAFs.

Cell-associated TGFβ1/proTGFβ1: The term refers to TGFβ1 or its signaling complex (e.g., pro/latent TGFβ1) that is membrane-bound (e.g., tethered to cell surface). Typically, such cell is an immune cell. TGFβ1 that is presented by GARP or LRRC33 is a cell-associated TGFβ1. GARP and LRRC33 are transmembrane presenting molecules that are expressed on cell surface of certain cells. GARP-proTGFβ1 and LRRC33-proTGFβ1 may be collectively referred to as “cell-associated” (or “cell-surface”) proTGFβ1 complexes, that mediate cell-associated (e.g., immune cell-associated) TGFβ1 activation/signaling. The term also includes recombinant, purified GARP-proTGFβ1 and LRRC33-proTGFβ1 complexes in solution (e.g., in vitro assays) which are not physically attached to cell membranes. Average KD values of an antibody (or its fragment) to a GARP-proTGFβ1 complex and an LRRC33-proTGFβ1 complex may be calculated to collectively represent affinities for cell-associated (e.g., immune cell-associated) proTGFβ1 complexes. Human counterpart of a presenting molecule or presenting molecule complex may be indicated by an “h” preceding the protein or protein complex, e.g., “hGARP,” “hGARP-proTGFβ1,” hLRRC33” and “hLRRC33-proTGFβ1.” In addition to blocking release of active TGFβ1 growth factor from cell-tethered complexes, cell-associated proTGFβ1 may be a target for internalization (e.g., endocytosis) and/or cell killing such as ADCC, ADCP, or ADC-mediated depletion of the target cells expressing such cell surface complexes.

Checkpoint inhibitor: In the context of this disclosure, checkpoint inhibitors refer to immune checkpoint inhibitors and carries the meaning as understood in the art. A “checkpoint inhibitor therapy” or “checkpoint blockade therapy” is one that targets a checkpoint molecule to partially or fully alter its function. Typically, a checkpoint is a receptor molecule on a T cell or NK cell, or a corresponding cell surface ligand on an antigen-presenting cell (APC) or tumor cell. Without being bound by theory, immune checkpoints are activated in immune cells to prevent inflammatory immunity developing against the “self”. Therefore, changing the balance of the immune system via checkpoint inhibition may allow it to be fully activated to detect and eliminate the cancer. The best known inhibitory receptors implicated in control of the immune response are cytotoxic T-lymphocyte antigen-4 (CTLA-4), programmed cell death protein 1 (PD-1), programmed cell death receptor ligand 1 (PD-L1), T-cell immunoglobulin domain and mucin domain-3 (TIM3), lymphocyte-activation gene 3 (LAG3), killer cell immunoglobulin-like receptor (KIR), glucocorticoid-induced tumor necrosis factor receptor (GITR) and V-domain immunoglobulin (Ig)-containing suppressor of T-cell activation (VISTA). Non-limiting examples of checkpoint inhibitors include: Nivolumab, Pembrolizumab, cemiplimab, BMS-936559, Atezolizumab, Avelumab, Durvalumab, Ipilimumab, Tremelimumab, IMP-321 (Eftilagimod alpha or ImmuFact®), BMS-986016 (Relatlimab), budigalimab (ABBV-181, anti-PD-1 antibody), and Lirilumab. Keytruda® is an example of anti-PD-1 antibodies. Budigalimab is a humanized, recombinant IgG1 monoclonal antibody targeting PD-1, that has been shown to be equally safe and well-tolerated in patients with HNSCC and NSCLC in a phase I study (Italiano et al., Cancer Immunology, Immunotherapy (2022) 71:417-431). Opdivo® is one example of an anti-PD-1 antibody. Therapies or therapeutic regimens that employ one or more of immune checkpoint inhibitors may be referred to as checkpoint blockade therapy (CBT) or checkpoint inhibitor therapy (CPI).

Clinical benefit: As used herein, the term “clinical benefits” is intended to include both efficacy and safety of a therapy. Thus, therapeutic treatment that achieves a desirable clinical benefit is both efficacious (e.g., achieves therapeutically beneficial effects) and safe (e.g., with tolerable or acceptable levels of toxicities or adverse events).

Clinical benefit rate: The term “clinical benefit rate” refers to the percentage of patients with complete response, partial response, or at least months of stable disease as a result of their therapy. This term may be used to characterize the tumorstatic efficacy of a therapy and/or ability to promote stable disease.

Combination therapy: “Combination therapy” refers to treatment regimens for a clinical indication that comprise two or more therapeutic agents. Thus, the term refers to a therapeutic regimen in which a first therapy comprising a first composition (e.g., active ingredient) is administered in conjunction with at least a second therapy comprising a second composition (active ingredient) to a patient, intended to treat the same or overlapping disease or clinical condition. The term may further encompass a therapeutic regimen in which a first therapy comprising a first composition (e.g., active ingredient) is administered in conjunction with a second therapy comprising a second composition (e.g., active ingredient such as a checkpoint inhibitor), a third therapy comprising a third composition (e.g., active ingredient such as a chemotherapy), or more (e.g., additional distinct active ingredients). The first, second, and (optionally additional) compositions may act on the same cellular target, or discrete cellular targets. The phrase “in conjunction with,” in the context of combination therapies, means that therapeutic effects of a first therapy overlaps temporally and/or spatially with therapeutic effects of a second and additional therapy in the subject receiving the combination therapy. The first, second, and/or additional compositions may be administered concurrently (e.g., simultaneously), separately, or sequentially. Thus, the combination therapies may be formulated as a single formulation for concurrent administration, or as separate formulations, for sequential, concurrent, or simultaneous administration of the therapies. When a subject who has been treated with a first therapy to treat a disease is administered with a second and additional therapies to treat the same disease, the second and additional therapies may be referred to as an add-on therapy or adjunct therapy.

Combinatory or combinatorial epitope: A combinatorial epitope is an epitope that is recognized and bound by a combinatorial antibody at a site (i.e., antigenic determinant) formed by non-contiguous portions of a component or components of an antigen, which, in a three-dimensional structure, come together in close proximity to form the epitope. Thus, antibodies of the disclosure may bind an epitope formed by two or more components (e.g., portions or segments) of a pro/latent TGFβ1 complex. A combinatory epitope may comprise amino acid residue(s) from a first component of the complex, and amino acid residue(s) from a second component of the complex, and so on. Each component may be of a single protein or of two or more proteins of an antigenic complex. A combinatory epitope is formed with structural contributions from two or more components (e.g., portions or segments, such as amino acid residues) of an antigen or antigen complex.

Compete or cross-compete; cross-block: The term “compete” when used in the context of antigen binding proteins (e.g., an antibody or antigen binding portion thereof) that compete for the same epitope means competition between antigen binding proteins as determined by an assay in which the antigen binding protein being tested prevents or inhibits (e.g., reduces) specific binding of a reference antigen binding protein to a common antigen (e.g., TGFβ1 or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay; solid phase direct biotin-avidin EIA; solid phase direct labeled assay, and solid phase direct labeled sandwich assay. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more when the competing antibody is present in excess. In some embodiments, an SPR (e.g., Biacore) assay is used to determine competition. In some embodiments, a BLI (e.g., Octet®) assay is used to determine competition

In some embodiments, a first antibody or antigen-binding portion thereof and a second antibody or antigen-binding portion thereof “cross-block” with each other with respect to the same antigen, for example, as assayed by Biolayer Interferometry (such as Octet®) or by surface plasmon resonance (such as Biacore System), using standard test conditions, e.g., according to the manufacturer's instructions (e.g., binding assayed at room temperature, ~20-25° C.). In some embodiments, the first antibody or fragment thereof and the second antibody or fragment thereof may have the same epitope. In other embodiments, the first antibody or fragment thereof and the second antibody or fragment thereof may have non-identical but overlapping epitopes. In yet further embodiments, the first antibody or fragment thereof and the second antibody or fragment thereof may have separate (different) epitopes which are in close proximity in a three-dimensional space, such that antibody binding is cross-blocked via steric hindrance. “Cross-block” means that binding of the first antibody to an antigen prevents binding of the second antibody to the same antigen, and similarly, binding of the second antibody to an antigen prevents binding of the first antibody to the same antigen.

Antibody binning (sometimes referred to as epitope binning or epitope mapping) may be carried out to characterize and sort a set (e.g., “a library”) of monoclonal antibodies made against a target protein or protein complex (i.e., antigen). Such antibodies against the same target are tested against all other antibodies in the library in a pairwise fashion to evaluate if antibodies block one another's binding to the antigen. Closely related binning profiles indicate that the antibodies have the same or closely related (e.g., overlapping) epitope and are “binned” together. Binning provides useful structure-function profiles of antibodies that share similar binding regions within the same antigen because biological activities (e.g., intervention; potency) effectuated by binding of an antibody to its target is likely to be carried over to another antibody in the same bin. Thus, among antibodies within the same epitope bin, those with higher affinities (lower KD) typically have greater potency.

In some embodiments, an antibody that binds the same epitope as Ab6 binds a proTGFβ1 complex such that the epitope of the antibody includes one or more amino acid residues of Region 1, Region 2 and Region 3, identified as the binding region of Ab6.

Complementary determining region (CDR): As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al., (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al., (1989) Nature 342: 877-883) found that certain sub-portions within Kabat CDRs adopt nearly identical peptide backbone conformations, despite having great diversity at the level of amino acid sequence. These sub-portions were designated as L1, L2 and L3 or H1, H2 and H3, or L-CDR1, L-CDR2 and L-CDR3 or H-CDR1, H-CDR2 and H-CDR3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding (see, for example: Lu X et al., Mabs. 2019 January; 11(1):45-57). The methods used herein may utilize CDRs defined according to any of these systems, although certain embodiments use Kabat or Chothia defined CDRs.

Conformational epitope: A conformational epitope is an epitope that is recognized and bound by a conformational antibody in a three-dimensional conformation, but not in an unfolded peptide of the same amino acid sequence. A conformational epitope may be referred to as a conformation-specific epitope, conformation-dependent epitope, or conformation-sensitive epitope. A corresponding antibody or fragment thereof that specifically binds such an epitope may be referred to as conformation-specific antibody, conformation-selective antibody, or conformation-dependent antibody. Binding of an antigen to a conformational epitope depends on the three-dimensional structure (conformation) of the antigen or antigen complex.

Constant region/domain: An immunoglobulin constant domain refers to a heavy or light chain constant domain. Human IgG heavy chain and light chain constant domain amino acid sequences are known in the art.

Context-biased: As used herein, “context-biased antibodies” refer to a type of conformational antibodies that binds an antigen with differential affinities when the antigen is associated with (i.e., bound to or attached to) an interacting protein or a fragment thereof. Thus, a context-biased antibody that specifically binds an epitope within proTGFβ1 may bind LTBP1-proTGFβ1, LTBP3-proTGFβ1, GARP-proTGFβ1 and LRRC33-proTGFβ1 with different affinities. For example, an antibody is said to be “matrix-biased” if it has higher affinities for matrix-associated proTGFβ1 complexes (e.g., LTBP1-proTGFβ1 and LTBP3-proTGFβ1) than for cell-associated proTGFβ1 complexes (e.g., GARP-proTGFβ1 and LRRC33-proTGFβ1). Relative affinities of [matrix-associated complexes]: [cell-associated complexes] may be obtained by taking average K_{D} values of the former, taking average K_{D} values of the latter, and calculating the ratio of the two, as exemplified herein. A context-biased antibody may also be biased for or against one presenting molecule-proTGFβ1 complex relative to the other presenting molecule-proTGFβ1 complexes, such that the affinity (as measured by KD) for the former is more than 10-fold weaker or greater than the average of the latter, respectively.

Context-independent: According to the present disclosure, “a context-independent antibody” that binds proTGFβ1 has equivalent affinities across the four known presenting molecule-proTGFβ1 complexes, namely, LTBP1-proTGFβ1, LTBP3-proTGFβ1, GARP-proTGFβ1 and LRRC33-proTGFβ1. Context-independent antibodies disclosed in the present application may also be characterized as unbiased or balanced. Typically, context-independent antibodies show equivalent (i.e., no more than five-fold bias in) affinities, such that relative ratios of measured KD values between matrix-associated complexes and cell-associated complexes are no greater than 5 as measured by a suitable in vitro binding assay, such as surface plasmon resonance, Biolayer Interferometry (BLI), and/or solution equilibrium titration (e.g., MSD-SET). In a preferred embodiment, surface plasmon resonance is used.

Dissociation rate: The term dissociation rate as used herein has the meaning understood by the skilled artisan in the pertinent art (e.g., antibody technology) as refers to a kinetics parameter measured by how fast/slow a ligand (e.g., antibody or fragment) dissociates from its binding target (e.g., antigen). Dissociation rate is also referred to as the “off” rate (“k_{OFF}”). Relative on/off rates between an antibody and its antigen (i.e., k_{ON} and k_{OFF}) determine the overall strength of the interaction, or affinity, typically expressed as a dissociation constant, or K_{D}. Therefore, equivalent affinities (e.g., K_{D} values) may be achieved by having fast association (high k_{ON}), slow dissociation (low k_{OFF}), or contribution from both factors. Monovalent interactions may be measured by the use of monovalent antigen-binding molecules/fragments, such as fAb (Fab), whilst divalent interactions may be measured by the use of divalent antigen-binding molecules such as whole immunoglobulins (e.g., IgGs). Dissociation rates can be experimentally measured in suitable in vitro binding assays, such as OCTET®- and BIACORE®-based systems.

ECM-associated TGFβ1/proTGFβ1: The term refers to TGFβ1 or its signaling complex (e.g., pro/latent TGFβ1) that is a component of (e.g., deposited into) the extracellular matrix. TGFβ1 that is presented by LTBP1 or LTBP3 is an ECM-associated TGFβ1, namely, LTBP1-proTGFβ1 and LTBP3-proTGFβ1, respectively. LTBPs are critical for correct deposition and subsequent bioavailability of TGFβ in the ECM, where fibrillin (Fbn) and fibronectin (FN) are believed to be the main matrix proteins responsible for the association of LTBPs with the ECM. Such matrix-associated latent complexes are enriched in connective tissues, as well as certain disease-associated tissues, such as tumor stroma and fibrotic tissues. Human counterpart of a presenting molecule or presenting molecule complex may be indicated by an “h” preceding the protein or protein complex, e.g., “hLTBP1,” “hLTBP1-proTGFβ1,” hLTBP3” and “hLTBP3-proTGFβ1.” Average KD values of an antibody (or its fragment) to an LTBP1-proTGFβ1 complex and an LTBP3-proTGFβ1 complex may be calculated to collectively represent affinities for ECM-associated (or matrix-associated) proTGFβ1 complexes.

Effective amount: The terms “effective” and “therapeutically effective” refer to the ability or an amount to sufficiently produce a detectable change in a parameter of a disease, e.g., a slowing, pausing, reversing, diminution, or amelioration in a symptom or downstream effect of the disease. The term encompasses but does not require the use of an amount that completely cures a disease. An “effective amount” (or therapeutically effective amount, or therapeutic dose) may be a dosage or dosing regimen that achieves a statistically significant clinical benefit (e.g., efficacy) in a patient population. For example, for an antibody that has been shown to be efficacious at doses between 3 mg/kg and 30 mg/kg in preclinical models, the effective amount can be said to be between about 3-30 mg/kg. For example, Ab6 has been shown to be efficacious at doses as low as 3 mg/kg and as high as 30 mg/kg in preclinical models. The term “minimum effective dose” or “minimum effective amount” refers to the lowest amount, dosage, concentration, or dosing regimen that achieves a detectable change in a parameter of a disease, e.g., a statistically significant clinical benefit. References herein to a dose of an agent (e.g., a dose of a TGFβ1 inhibitor) may be a therapeutically effective dose, as described herein. In a clinical setting, such as human clinical trials, the term “pharmacological active dose (PAD)” may be used to refer to effective dosage. Effective amounts may be expressed in terms of doses being administered or in terms of exposure levels achieved as a result of administration (e.g., serum concentrations).

Effective tumor control: The term “effective tumor control” may be used to refer to a degree of tumor regression achieved in response to treatment, where, for example, the tumor is regressed by a defined fraction (such as <25%) of an endpoint tumor volume. For instance, in a particular model, if the endpoint tumor volume is set at 2,000 mm³, effective tumor control is achieved if the tumor is reduced to less than 500 mm³ assuming the threshold of <25%. Therefore, effective tumor control encompasses complete regression. Clinically, effective tumor control can be measured by objective response, which includes partial response (PR) and complete response (CR) as determined by art-recognized criteria, such as RECIST v1.1 and corresponding iRECIST (iRECIST v1.1). In some embodiments, effective tumor control in clinical settings also includes stable disease, where tumors that are typically expected to grow at certain rates are prevented from such growth by the treatment, even though shrinkage is not achieved.

Effector T cells: Effector T cells, as used herein, are T lymphocytes that actively respond immediately to a stimulus, such as co-stimulation and include, but are not limited to, CD4+ T cells (also referred to as T helper or Th cells) and CD8+ T cells (also referred to as cytotoxic T cells). Th cells assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including Th1, Th2, Th3, Th17, Th9, or TFh, which secrete different cytokines to facilitate different types of immune responses. Signaling from the APC directs T cells into particular subtypes. Cytotoxic (Killer). Cytotoxic T cells (TC cells, CTLs, T-killer cells, killer T cells), on the other hand, destroy virus-infected cells and cancer cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Cytotoxic effector cell (e.g., CD8+ cells) markers include, e.g., perforin and granzyme B.

Endpoint: In studies aimed to assess effectiveness (e.g., clinical benefit or improvements) of a therapy, such as in clinical trials for a cancer therapy, endpoints represent the measures of predetermined parameters indicative of treatment effects. In oncology, suitable endpoints may include overall survival, disease-free survival (DFS), event-free survival (EFS), progression-free survival (PFS), objective response rate (ORR), complete response (CR), partial response (PR), time to progression (TTP), as well as patient-reported outcomes (e.g., symptom assessment) and biomarker assessment such as blood or body fluid-based assessments.

Epithelial hyperplasia: The term “epithelial hyperplasia” refers to an increase in tissue growth resulting from proliferation of epithelial cells. As used herein, epithelial hyperplasia refers to the undesired toxicity resulting from TGFβ inhibition which may include, but is not limited to, abnormal growth of epithelial cells in the oral cavity, esophagus, breast, and ovary.

Epitope: The term “epitope” may be also referred to as an antigenic determinant, is a molecular determinant (e.g., polypeptide determinant) that can be specifically bound by a binding agent, immunoglobulin, or T-cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody may specifically bind an antigen when it recognizes its target antigen in a complex mixture of proteins and/or macromolecules. For example, antibodies are said to “bind to the same epitope” if the antibodies cross-compete (one prevents the binding or modulating effect of the other).

Equivalent affinity: In the context of the present disclosure, the term “equivalent affinity/affinities” is intended to mean: i) the antibody binds matrix-associated proTGFβ1 complexes and cell-associated proTGFβ1 complexes with less than five-fold bias in affinity, as measured by suitable in vitro binding assays, such as solution equilibrium titration (such as MSD-SET), Biolayer Interferometry (such as Octet®) or surface plasmon resonance (such as Biacore System; and/or, ii) relative affinities of the antibody for the four complexes are uniform in that: either, the lowest affinity (highest KD numerical value) that the antibody shows among the four antigen complexes is no more than five-fold less than the average value calculated from the remaining three affinities; or, the highest affinity (lowest KD numerical value) that the antibody shows among the four antigen complexes is no more than five-fold greater than the average calculated from the remaining three affinities. Antibodies with equivalent affinities may achieve more uniform inhibitory effects, irrespective of the particular presenting molecule associated with the proTGFβ1 complex (hence “context-independent”). In some embodiments, bias observed in average affinities between matrix-associated complexes and cell-associated complexes is no more than three-fold. In preferred embodiments, affinities are measured by surface plasmon resonance (e.g., a Biacore system). Such methods are to be carried out using standard test conditions, e.g., according to the manufacturer's instructions.

Extended Latency Lasso: The term “Extended Latency Lasso” as used herein refers to a portion of the prodomain that comprises Latency Lasso and Alpha-2 Helix, e.g., LASPPSQGEVPPGPLPEAVLALYNSTR (SEQ ID NO: 1127). In some embodiments, Extended Latency Lasso further comprises a portion of Alpha-1 Helix, e.g., LVKRKRIEA (SEQ ID NO: 1132) or a portion thereof.

Fibrosis: The term “fibrosis” or “fibrotic condition/disorder” refers to the process or manifestation characterized by the pathological accumulation of extracellular matrix (ECM) components, such as collagens, within a tissue or organ. Indeed, collagen accumulation is a hallmark of fibrosis. According to some embodiments, the fibrosis is lung (also referred to as pulmonary) fibrosis.

Pulmonary fibrosis: The term “pulmonary fibrosis” or “lung fibrosis” as used in the context of the present disclosure refers to the formation of excess fibrous connective tissue in the lung. According to some embodiments, pulmonary fibrosis may be a secondary effect of other lung diseases. Examples of such diseases include autoimmune disorders, viral infections and bacterial infections (such as tuberculosis). Pulmonary fibrosis may also be idiopathic, with cigarette smoking, environmental factors (e.g., occupational exposure to gases, smoke, chemicals or dusts) or genetic predisposition thought to be risk factors.

Fibrotic microenvironment: The term “fibrotic microenvironment” refers to a local disease niche within a tissue, in which fibrosis occurs in vivo. The fibrotic microenvironment may comprise disease-associated molecular signature (a set of chemokines, cytokines, etc.), disease-associated cell populations (such as activated macrophages, MDSCs, etc.) as well as disease-associated ECM environments (alterations in ECM components and/or structure). Fibrotic microenvironment is thought to support the transition of fibroblast to a-smooth muscle actin-positive myofibroblast in a TGFβ-dependent manner. Fibrotic microenvironment may be further characterized by the infiltration of certain immune cells (such as macrophages and MDSCs).

Finger-1 (of TGFβ1 Growth Factor): As used herein, “Finger-1” is a domain within the TGFβ1 growth factor domain. In its unmutated form, Finger-1 of human proTGFβ1 contains the following amino acid sequence: CVRQLYIDFRKDLGWKWIHEPKGYHANFC (SEQ ID NO: 1124). In the 3D structure, the Finger-1 domain comes in close proximity to Latency Lasso.

Finger-2 (of TGFβ1 Growth Factor): As used herein, “Finger-2” is a domain within the TGFβ1 growth factor domain. In its unmutated form, Finger-2 of human proTGFβ1 contains the following amino acid sequence: CVPQALEPLPIVYYVGRKPKVEQLSNMIVRSCKCS (SEQ ID NO: 1125). Finger-2 includes the “binding region 6”, which spatially lies in close proximity to Latency Lasso.

Gamma delta (γδ) T cells: As used herein, the term “gamma delta (γδ) T cells” refers to a subgroup of unconventional T cells with functions not restricted to MHC-mediated antigen presentation. γδ T cells are actively recruited to tumor microevironments (TMEs), and are generally considered cytotoxic and antitumor lymphocytes, but some γδ T cell subsets, especially those expressing IL-17, CD39, or FOXP3, are immunosuppressive or tumor-promoting cells (Park et al., Exp Mol Med, 2021 March, 53(3):318-327). TGFβ can also inhibit the antitumor cytotoxicity of human Vγ9Vδ2 T cells (Rafia et al., Front Immunol, 2023 Jan. 19, 13:1066336).

GARP-TGFβ1/GARP-proTGFβ1 complex: As used herein, the term “GARP-TGFβ1 complex” “(or “GARP-proTGFβ1 complex”) refers to a protein complex comprising a pro-protein form or latent form of a transforming growth factor-31 (TGFβ1) protein and a glycoprotein-A repetitions predominant protein (GARP) or fragment or variant thereof. In some embodiments, a pro-protein form or latent form of TGFβ1 protein may be referred to as “pro/latent TGFβ1 protein”. In some embodiments, a GARP-TGFβ1 complex comprises GARP covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a GARP-TGFβ1 complex comprises GARP non-covalently linked with pro/latent TGFβ1. In some embodiments, a GARP-TGFβ1 complex is a naturally-occurring complex, for example a GARP-TGFβ1 complex in a cell. The term “hGARP” denotes human GARP.

High-affinity: As used herein, the term “high-affinity” as in “a high-affinity proTGFβ1 antibody” refers to in vitro binding activities having a K_{D} value of ≤5 nM, more preferably ≤1 nM. Thus, a high-affinity, context-independent proTGFβ1 antibody encompassed by the disclosure herein has a K_{D} value of ≤5 nM, more preferably ≤1 nM, towards each of the following antigen complexes: LTBP1-proTGFβ1, LTBP3-proTGFβ1, GARP-proTGFβ1 and LRRC33-proTGFβ1.

Human antibody: The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Humanized antibody: The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody. As used herein, the term “substantially” in the context of a CDR refers to a CDR having an amino acid sequence at least 80%, at least 85%, at least 90%, at least 95%, at least 96%, at least 97%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment a humanized antibody also comprises at least a portion of an immunoglobulin Fc region, typically that of a human immunoglobulin. In some embodiments a humanized antibody contains the light chain as well as at least the variable domain of a heavy chain. The antibody also may include the CH1, hinge, CH2, CH3, and CH4 regions of the heavy chain. In some embodiments a humanized antibody only contains a humanized light chain. In some embodiments a humanized antibody only contains a humanized heavy chain. In specific embodiments a humanized antibody only contains a humanized variable domain of a light chain and/or humanized heavy chain.

Immune-excluded or immuno-excluded tumor As used herein, tumors characterized as “immune excluded” are devoid of or substantially devoid of intratumoral anti-tumor lymphocytes. For example, tumors with poorly infiltrated T cells may have T cells that surround the tumor, e.g., the external perimeters of a tumor mass and/or near the vicinity of vasculatures (“perivascular”) of a tumor, which nevertheless fail to effectively swarm into the tumor to exert cytotoxic function against cancer cells. In other situations, tumors fail to provoke a strong immune response (so-called “immune desert” tumors) such that few T cells are present near and in the tumor environment. In contrast to immune-excluded tumors, tumors that are infiltrated with anti-tumor lymphocytes (“immune-infiltrated tumors”), such as CD8+ T cells, are sometimes characterized as “hot” or “inflamed” tumors; such tumors are more likely to be responsive to and therefore may be the target of immune checkpoint blockade therapies (“CBTs”), although some immune-infiltrated tumors are also resistant or refractory to checkpoint blockade therapies. Typically, however, only a fraction of patients responds to a CBT, usually due to immune exclusion that renders the tumor resistant to the CBT.

Immune safety (assessment): As used herein, the term refers to safety assessment related to immune responses (immune activation), Acceptable immune safety criteria include no significant cytokine release as determined by in vitro or in vivo cytokine release testing (e.g., assays); and no significant platelet aggregation, activation as determined with human platelets. Statistical significance in these studies may be determined against a suitable control as reference. For example, for a test molecule which is a human monoclonal antibody, a suitable control may be an immunoglobulin of the same subtype, e.g., an antibody of the same subtype known to have a good safety profile in a human.

Immunosuppression, immune suppression, immunosuppressive: The terms refer to the ability to suppress immune cells, such as T cells, NK cells and B cells. The gold standard for evaluating immunosuppressive function is the inhibition of T cell activity, which may include antigen-specific suppression and non-specific suppression. Regulatory T cells (Tregs), subsets of gamma delta (γδ) T cells, and MDSCs may be considered immunosuppressive cells. M2-polarized macrophages (e.g., disease-localized macrophages such as TAMs and FAMs) may also be characterized as immunosuppressive. A cancer, e.g., a solid tumor, having an immunosuppressive phenotype may accordingly comprise infiltrated Tregs, MDSCs, and/or macrophages, e.g., a high level of infiltrated Tregs, MDSCs, and/or macrophages. A cancer having an immunosuppressive phenotype may also be associated with elevated levels of circulating MDSCs, e.g., as compared to a healthy subject, or if circulating MDSCs (e.g., circulating gMDSCs) are detectable, such as above 1% of the white blood cell component/PBMC component.

Immunological memory: Immunological memory refers to the ability of the immune system to quickly and specifically recognize an antigen that the body has previously encountered and initiate a corresponding immune response. Generally, these are secondary, tertiary, and other subsequent immune responses to the same antigen. Immunological memory is responsible for the adaptive component of the immune system, special T and B cells—the so-called memory T and B cells. Antigen-naïve T cells expand and differentiate into memory and effector T cells after they encounter their cognate antigen within the context of an MHC molecule on the surface of a professional antigen presenting cell (e.g., a dendritic cell). The single unifying theme for all memory T cell subtypes is that they are long-lived and can quickly expand to large numbers of effector T cells upon re-exposure to their cognate antigen. By this mechanism they provide the immune system with “memory” against previously encountered pathogens. Memory T cells may be either CD4+ or CD8+ and usually express CD45RO. In a preclinical setting, immunological memory may be tested in a tumor rechallenge paradigm.

Inhibit or inhibition of: The term “inhibit” or “inhibition of,” as used herein, means to reduce by a measurable amount, and can include but does not require complete prevention or inhibition.

Isoform-non-specific/Isoform-non-selective: The term “isoform non-specific” or “isoform non-selectivity” refers to an agent's ability to bind to more than one structurally related isoforms. An isoform-non-specific TGFβ inhibitor exerts its inhibitory activity toward more than one isoform of TGFβ, such as TGFβ1/3, TGFβ1/2, TGFβ2/3, and TGFβ1/2/3.

Isoform-specific/selective: The term “isoform specificity” or “isoform selectivity” refers to an agent's ability to discriminate one isoform over other structurally related isoforms (i.e., isoform selectivity). An isoform-specific TGFβ inhibitor exerts its inhibitory activity towards one isoform of TGFβ but not the other isoforms of TGFβ at a given concentration. For example, an isoform-specific TGFβ1 antibody selectively binds TGFβ1. A TGFβ1-specific inhibitor (antibody) preferentially targets (binds thereby inhibits) the TGFβ1 isoform over TGFβ2 or TGFβ3 with substantially greater affinity. For example, the selectivity in this context may refer to at least a 10-fold, 100-fold, 500-fold, 1000-fold, or greater difference in respective affinities as measured by an in vitro binding assay such as BLI (Octet®) or preferably SPR (Biacore®). In some embodiments, the selectivity is such that the inhibitor when used at a dosage effective to inhibit TGFβ1 in vivo does not inhibit TGFβ2 and TGFβ3. For instance, an antibody may preferentially bind TGFβ1 at affinity of ~1 pM, while the same antibody may bind TGFβ2 and/or TGFβ3 at ~0.5-50 nM. For such an inhibitor to be useful as a therapeutic, dosage to achieve desirable effects (e.g., therapeutically effective amounts) must fall within the window within which the inhibitor can effectively inhibit the TGFβ1 isoform without inhibiting TGFβ2 or TGFβ3. In some embodiments, a TGFβ1-selective inhibitor is a pharmacological agent that interferes with the function or activities of TGFβ1, but not of TGFβ2 and/or TGFβ3, irrespective of the mechanism of action. The terms “isoform-specific” and “isoform-selective” are used interchangeably herein.

Isolated: An “isolated” antibody as used herein, refers to an antibody that is substantially free of other antibodies having different antigenic specificities. In some embodiments, an isolated antibody is substantially free of other unintended cellular material and/or chemicals.

Large Latent Complex: The term “large latent complex” (“LLC”) in the context of the present disclosure refers to a complex comprised of a proTGFβ1 dimer bound to so-called a presenting molecule. Thus, a large latent complex is a presenting molecule-proTGFβ1 complex, such as LTBP1-proTGFβ1, LTBP3-proTGFβ1, GARP-proTGFβ1 and LRRC33-proTGFβ1. Such complexes may be formed in vitro using recombinant, purified components capable of forming the complex. For screening purposes, presenting molecules used for forming such LLCs need not be full length polypeptides; however, the portion of the protein capable of forming disulfide bonds with the proTGFβ1 dimer complex via the cysteine residues near its N-terminal regions is typically required.

Latency associated peptide (LAP): LAP is so-called the “prodomain” of proTGFβ1. As described in more detail herein, LAP is comprised of the “Straight Jacket” domain and the “Arm” domain. Straight Jacket itself is further divided into the Alpha-1 Helix and Latency Lasso domains.

Latency Lasso: As used herein, “Latency Lasso,” sometimes also referred to as Latency Loop, is a domain flanked by Alpha-1 Helix and the Arm within the prodomain of proTGFβ1. In its unmutated form, Latency Lasso of human proTGFβ1 comprises the amino acid sequence: LASPPSQGEVPPGPL (SEQ ID NO: 1126) which is spanned by Region 1. As used herein, the term Extended Latency Lasso region” refers to the Latency Lasso together with its immediate C-terminal motif referred to as Alpha-2 Helix (α2-Helix) of the prodomain. The proline residue that is at the C-terminus of the Latency Lasso provides the perpendicular “turn” like an “elbow” that connects the lasso loop to the α2-Helix. Certain high affinity TGFβ1 activation inhibitors bind at least in part to Latency Lasso or a portion thereof to confer the inhibitory potency (e.g., the ability to block activation), wherein optionally the portion of the Latency Lasso is ASPPSQGEVPPGPL (SEQ ID NO: 1170). In some embodiments, the antibodies of the present disclosure bind a proTGFβ1 complex at ASPPSQGEVPPGPL (SEQ ID NO: 1170) or a portion thereof. Certain high affinity TGFβ1 activation inhibitors bind at least in part to Extended Latency Lasso or a portion thereof to confer the inhibitory potency (e.g., the ability to block activation), wherein optionally the portion of the Extended Latency Lasso is KLRLASPPSQGEVPPGPLPEAVL (SEQ ID NO: 1142) or LASPPSQGEVPPGPLPEAVLALYNSTR (SEQ ID NO: 271).

Localized: In the context of the present disclosure, the term “localized” (as in “localized tumor”, “disease-localized” etc.) refers to anatomically isolated or isolatable abnormalities, such as solid malignancies, as opposed to systemic disease. Certain leukemia, for example, may have both a localized component (for instance the bone marrow) and a systemic component (for instance circulating blood cells) to the disease.

LRRC33-TGFβ1/LRRC33-proTGFβ1 complex: As used herein, the term “LRRC33-TGFβ1 complex” (or “LRRC33-proTGFβ1 complex”) refers to a complex between a pro-protein form or latent form of transforming growth factor-31 (TGFβ1) protein and a Leucine-Rich Repeat-Containing Protein 33 (LRRC33; also known as Negative Regulator of Reactive Oxygen Species or NRROS) or fragment or variant thereof. In some embodiments, a LRRC33-TGFβ1 complex comprises LRRC33 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LRRC33-TGFβ1 complex comprises LRRC33 non-covalently linked with pro/latent TGFβ1. In some embodiments, a LRRC33-TGFβ1 complex is a naturally-occurring complex, for example a LRRC33-TGFβ1 complex in a cell. The term “hLRRC33” denotes human LRRC33. In vivo, LRRC33 and LRRC33-containing complexes on cell surface may be internalized. LRRC33 is expressed on a subset of myeloid cells, including M2-polarized macrophages (such as TAMs) and MDSCs. MDSCs that express LRRC33 on cell surface include tumor-associated MDSCs and circulatory MDSCs. LRRC33-expressing tumor-associated MDSCs may include gMDSCs. LRRC33-expressing MDSCs in circulation may include g-MDSCs.

LTBP1-TGFβ1/LTBP1-proTGFβ1 complex: As used herein, the term “LTBP1-TGFβ1 complex” (or “LTBP1-proTGFβ1 complex”) refers to a protein complex comprising a pro-protein form or latent form of transforming growth factor-31 (TGFβ1) protein and a latent TGF-beta binding protein 1 (LTBP1) or fragment or variant thereof. In some embodiments, a LTBP1-TGFβ1 complex comprises LTBP1 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LTBP1-TGFβ1 complex comprises LTBP1 non-covalently linked with pro/latent TGFβ1. In some embodiments, a LTBP1-TGFβ1 complex is a naturally-occurring complex, for example a LTBP1-TGFβ1 complex in a cell. The term “hLTBP1” denotes human LTBP1.

LTBP3-TGFβ1/LTBP3-proTGFβ1 complex: As used herein, the term “LTBP3-TGFβ1 complex” (or “LTBP3-proTGFβ1 complex”) refers to a protein complex comprising a pro-protein form or latent form of transforming growth factor-31 (TGFβ1) protein and a latent TGF-beta binding protein 3 (LTBP3) or fragment or variant thereof. In some embodiments, a LTBP3-TGFβ1 complex comprises LTBP3 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LTBP3-TGFβ1 complex comprises LTBP1 non-covalently linked with pro/latent TGFβ1. In some embodiments, a LTBP3-TGFβ1 complex is a naturally-occurring complex, for example a LTBP3-TGFβ1 complex in a cell. The term “hLTBP3” denotes human LTBP3.

M2 or M2-like macrophage: M2 macrophages represent a subset of activated or polarized macrophages and include disease-associated macrophages in both fibrotic and tumor microenvironments. Cell-surface markers for M2-polarized macrophages typically include CD206 and CD163 (i.e., CD206+/CD163+). Applicant recently discovered that the M2-polarized macrophages may also express cell-surface LRRC33. Activation of M2 macrophages is promoted mainly by IL-4, IL-13, IL-10 and TGFβ; they secrete the same cytokines that activate them (IL-4, IL-13, IL-10 and TGFβ). These cells have high phagocytic capacity and produce ECM components, angiogenic and chemotactic factors. The release of TGFβ by macrophages may perpetuate the myofibroblast activation, EMT and EndMT induction in the disease tissues, such as fibrotic tissue and tumor stroma. For example, M2 macrophages play a role in TGFβ-driven lung fibrosis and are also enriched in a number of tumors.

Matrix-associated proTGFβ1: LTBP1 and LTBP3 are presenting molecules that are components of the extracellular matrix (ECM). LTBP1-proTGFβ1 and LTBP3-proTGFβ1 may be collectively referred to as “ECM-associated” (or “matrix-associated”) proTGFβ1 complexes, that mediate ECM-associated TGFβ1 activation/signaling. The term also includes recombinant, purified LTBP1-proTGFβ1 and LTBP3-proTGFβ1 complexes in solution (e.g., in vitro assays) which are not physically attached to a matrix or substrate.

Maximally tolerated dose (MTD): The term MTD generally refers to, in the context of safety/toxicology considerations, the highest amount of a test article (such as a TGFβ1 inhibitor) evaluated with no-observed-adverse-effect level (NOAEL). For example, the NOAEL for Ab6 in rats was the highest dose evaluated (100 mg/kg), suggesting that the MTD for Ab6 is >100 mg/kg, based on a four-week toxicology study. The NOAEL for Ab6 in non-human primates was the highest dose evaluated (300 mg/kg), suggesting that the MTD for Ab6 in the non-human primates is >300 mg/kg, based on a four-week toxicology study.

Meso-Scale Discovery: “Meso-Scale Discovery” or “MSD” is a type of immunoassays that employs electrochemiluminescence (ECL) as a detection technique. Typically, high binding carbon electrodes are used to capture proteins (e.g., antibodies). The antibodies can be incubated with particular antigens, which binding can be detected with secondary antibodies that are conjugated to electrochemiluminescent labels. Upon an electrical signal, light intensity can be measured to quantify analytes in the sample.

Myelofibrosis: “Myelofibrosis,” also known as osteomyelofibrosis, is a relatively rare bone marrow proliferative disorder (e.g., cancer), which belongs to a group of diseases called myeloproliferative disorders and includes primary myelofibrosis and secondary myelofibrosis. Myelofibrosis is generally characterized by the proliferation of an abnormal clone of hematopoietic stem cells in the bone marrow and other sites results in fibrosis, or the replacement of the marrow with scar tissue. The term myelofibrosis encompasses primary myelofibrosis (PMF), also be referred to as chronic idiopathic myelofibrosis (cIMF) (the terms idiopathic and primary mean that in these cases the disease is of unknown or spontaneous origin), as well as secondary types of myelofibrosis, such as myelofibrosis that develops secondary to polycythemia vera (PV) or essential thrombocythaemia (ET). Myelofibrosis is a form of myeloid metaplasia, which refers to a change in cell type in the blood-forming tissue of the bone marrow, and often the two terms are used synonymously. The terms agnogenic myeloid metaplasia and myelofibrosis with myeloid metaplasia (MMM) are also used to refer to primary myelofibrosis. Myelofibrosis is characterized by mutations that cause upregulation or overactivation of the downstream JAK pathway.

Myeloid cells: In hematopoiesis, myeloid cells are blood cells that arise from a progenitor cell for granulocytes, monocytes, erythrocytes, or platelets (the common myeloid progenitor, that is, CMP or CFU-GEMM), or in a narrower sense also often used, specifically from the lineage of the myeloblast (the myelocytes, monocytes, and their daughter types), as distinguished from lymphoid cells, that is, lymphocytes, which come from common lymphoid progenitor cells that give rise to B cells and T cells. Certain myeloid cell types, their general morphology, typical cell surface markers, and their immune-suppressive ability in both mouse and human, are summarized below. In some embodiments, a human neutrophil can be identified by at least one (e.g., all) of the cell surface markers CD11b⁺, CD14⁻, CD15e, and CD66b⁺. In some embodiments, a human neutrophil is LOX-1⁻. In some embodiments, a human neutrophil is HLA-DR^{-/med}. In some embodiments, a classical human monocyte can be identified by at least one (e.g., all) of the cell surface markers CD14⁺ CD15⁻ CD16⁻ HLA-DR⁺. In some embodiments, a classical human monocyte is CD33 and/or CD11b⁺. In some embodiments, a classical human monocyte is CD16⁻. In some embodiments, an intermediate human monocyte can be identified by at least one (e.g., all) of the cell surface markers CD14⁺ CD15⁻ CD16⁺ HLA-DR⁺. In some embodiments, a non-classical human monocyte can be identified by at least one (e.g., all) of the cell surface markers CD14⁻ CD15⁻ CD16⁺ HLA-DR⁺. In some embodiments, a human M1 macrophage can be identified by at least one (e.g., all) of the cell surface markers CD15⁻ CD16⁺ CD80⁺ HLA-DR^{+/high} CD33+. In some embodiments, a human M1 macrophage is CD66b⁻. In some embodiments, a human M1 macrophage is CD11b⁺. In some embodiments, a human M macrophage is CD14⁻. In some embodiments, a human M2 macrophage can be identified by at least one (e.g., all) of the cell surface markers CD11b⁺ and CD15⁻. In some embodiments, a human M2 macrophage is CD206⁺. In some embodiments, a human M2 macrophage is CD163⁺. In some embodiments, a human M2 macrophage is HLA-DR⁺. In some embodiments, a human M2 macrophage is CD14⁻. In some embodiments, a human M2 macrophage is CD33⁺. In some embodiments, a human M2 macrophage is CD66b⁻.

| | | | |
|---|---|---|---|
| | | | Immune |
| Myeloid cells | Typical Morphology | Select surface phenotype | suppression |

Mouse

| | | | |
|---|---|---|---|
| Neutrophils | Round shape with a | CD11b⁺ Ly6G^{hi} Ly6C^{lo} | − |
| | segmented nucleus | | |
| Monocytes | Round shape with an | CD11b⁺ Ly6G⁻ Ly6C^{hi} | − |
| | indented nucleus | | |
| Macrophages | Round shape with | CD11b⁺ F4/80^{hi} Ly6G⁻ Ly6C^{lo} CD80⁺ | − |
| | pseudopodia | (M1) | |
| | | F4/80⁺ CD206⁺ CD163⁺ | − |
| | | LRRC33+ (M2) | |
| Dendritic cells | Dendritic shape | CD11b⁺ CD11c⁺ Ly6G⁻ Ly6C^{-/lo} | − |
| | with polypodia | (classical) | |
| | | CD11b⁻ CD11c⁺ Ly6G⁻ Ly6C⁻ | − |
| | | (classical) | |
| | | CD11b⁻ CD11c^{lo} Ly6G⁻ Ly6C⁺ PDCA-1⁺ | − |
| | | (plasmacytoid) | |
| Fibrocytes | Spindle shape | CD11b⁺ Coll⁺ Ly6G⁻ Ly6C⁺ | − |
| G-MDSCs | Round shape with a | CD11b⁺ Ly6G⁺ Ly6C^{lo} | + |
| (PMN-MDSCs) | banded nucleus | LRRC33+ | |
| M-MDSCs | Round shape with an | CD11b⁺ Ly6G⁻ Ly6C^{hi} | + |
| | indented nucleus | LRRC33+ | |

Human

| | | | |
|---|---|---|---|
| Neutrophils | Round shape with a | CD11b⁺ CD14⁻ CD15⁺ CD66b⁺ LOX-1⁻ | − |
| | segmented nucleus | | |
| Monocytes | Round shape with an | CD14⁺ CD15⁻ CD16⁻ HLA-DR⁺ | − |
| | indented nucleus | (classical) | |
| | | CD14⁺ CD15⁻ CD16⁺ HLA-DR⁺ | − |
| | | (intermediate) | |
| | | CD14⁻ CD15⁻ CD16⁺ HLA-DR⁺ | − |
| | | (non-classical) | |
| Macrophages | Round shape with | CD15⁻ CD16⁺ CD80⁺ HLA-DR⁺ CD33⁺ | − |
| | pseudopodia | (M1) | |
| | | CD11b⁺ CD15⁻ CD206⁺ CD163⁺ HLA-DR⁺ | +/− |
| | | (M2) | |
| Dendritic cells | Dendritic shape | CD14⁻ CD16⁻ CD1C⁺ CD83⁺ | − |
| | with polypodia | (classical) | |
| | | CD14⁻ CD16⁻ CD141⁺ CD83⁺ | − |
| | | (classical) | |
| | | CD14⁻ CD16⁻ CD303⁺ CD83⁺ | − |
| | | (plasmacytoid) | |
| Fibrocytes | Spindle shape | CD11b⁺ Coll⁺ CD13⁺ CD34⁺ CD45RO⁺ HLA-DR⁺ | − |
| G-MDSCs | Round shape with an | CD11b⁺ CD33⁺ CD14⁻ CD15⁺ CD66b⁺ LOX-1⁺ | + |
| (PMN-MDSCs) | annular nucleus | HLA-DR^{-/lo} | |
| M-MDSCs | Round shape with an | CD11b⁺ CD33⁺ CD14⁺ CD15⁻ HLA-DR^{-/lo} | + |
| | indented nucleus | | |

Myeloid-derived suppressor cell: Myeloid-derived suppressor cells (MDSCs) are a heterogeneous population of cells generated during various pathologic conditions and thought to represent a pathologic state of activation of monocytes and relatively immature neutrophils. MDSCs include at least two categories of cells termed i) “granulocytic” (G-MDSC) or polymorphonuclear (PMN-MDSC), which are phenotypically and morphologically similar to neutrophils; and ii) monocytic (M-MDSC) which are phenotypically and morphologically similar to monocytes. MDSCs are characterized by a distinct set of genomic and biochemical features, and can be distinguished by specific surface molecules. In certain embodiments, suitable cell surface markers for identifying MDSCs may include one or more of CD11b, CD33, CD14, CD15, HLA-DR and CD66b. For example, human G-MDSCs/PMN-MDSCs typically express the cell-surface markers CD11b, CD33, CD15 and CD66b. In some embodiments, human G-MDSCs may express low levels of the CD33 cell surface marker. Human G-MDSCs/PMN-MDSCs may also express LOX-1 and/or Arginase. By comparison, human M-MDSCs typically express the cell surface markers CD11b, CD33 and CD14. Additionally, both human G-MDSCs/PMN-MDSCs and M-MDSCs may also exhibit low levels or undetectable levels of HLA-DR. In certain embodiments. In certain embodiments, G-MDSCs may be differentiated from M-MDSCs based on the presence or absence of certain cell surface marker (e.g., CD14, CD15, and/or CD66b). The MDSCs may also express CD39 and CD73 to mediate adenosine signaling involved in organ fibrosis (such as liver fibrosis, and lung fibrosis), cancer and myelofibrosis). In addition, human M-MDSCs may also express HLA-DR. In some embodiments, G-MDSCs may be identified by the presence or elevated expression of surface markers CD11b, CD33, CD15, CD66b, and/or LOX-1, and the absence of CD14, whereas M-MDSCs may be identified by the presence or elevated expression of surface markers CD11 b, CD33, and/or CD14, and the absence of CD15. In some embodiments, M-MDSCs may be CD66b⁻. In addition to such cell-surface markers, MDSCs may be characterized by the ability to suppress immune cells, such as T cells, NK cells and B cells. Immune suppressive functions of MDSCs may include inhibition of antigen-non-specific function and inhibition of antigen-specific function. MDSCs, including tumor-associated MDSCs and MDSCs in circulation, can express cell surface LRRC33 and/or LRRC33-proTGFβ1. In some embodiments, a signal intensity of a cell surface marker may be categorized, or binned, as “low”, “medium”, or “high” based on normalization of signal intensity to reduce background and bleed through signals. In some embodiments, a signal intensity of a cell surface marker may be categorized based on cutoff thresholds provided in Table 14A. In some embodiments, a signal intensity of a cell surface marker may be determined by binary intensity selection. In some embodiments, the binary intensity selection comprises categorizing a signal intensity measured for a particular cell surface marker as “positive” or “negative.” In some embodiments, a signal intensity of a cell surface marker may be categorized based on the cutoff thresholds provided in Table 14B. In some embodiments, signal intensities of a set of surface markers may be determined by sequential application of signal filtering, where the signal intensity threshold for one or more surface markers is determined before the threshold is determined for one or more additional surface markers.

Myofibroblast: Myofibroblasts are cells with certain phenotypes of fibroblasts and smooth muscle cells and generally express vimentin, alpha-smooth muscle actin (α-SMA; human gene ACTA2) and paladin. In many disease conditions involving extracellular matrix dysregulations (such as increased matrix stiffness), normal fibroblast cells become de-differentiated into myofibroblasts in a TGFβ-dependent manner. Aberrant overexpression of TGFβ is common among myofibroblast-driven pathologies. TGFβ is known to promote myofibroblast differentiation, cell proliferation, and matrix production. Myofibroblasts or myofibroblast-like cells within the fibrotic microenvironment may be referred to as fibrosis-associated fibroblasts (or “FAFs”), and myofibroblasts or myofibroblast-like cells within the tumor microenvironment may be referred to as cancer-associated fibroblasts (or “CAFs”).

Pan-TGFβ inhibitor/pan-inhibition of TGFβ: The term “pan-TGFβ inhibitor” or “pan inhibitor of TGFβ” refers to any agent that is capable of inhibiting or antagonizing all three isoforms of TGFβ. Such an inhibitor may be a small molecule inhibitor of TGFβ isoforms, such as those known in the art. The term includes pan-TGFβantibody which refers to any antibody capable of binding to each of TGFβ isoforms, i.e., TGFβ1, TGFβ2, and TGFβ3. In some embodiments, a pan-TGFβantibody binds and neutralizes activities of all three isoforms, i.e., TGFβ1, TGFβ2, and TGFβ3. The antibody 1D11 (or the human analog fresolimumab (GC1008)) is a well-known example of a pan-TGFβ antibody that neutralizes all three isoforms of TGFβ. Examples of small molecule pan-TGFβ inhibitors include galunisertib (LY2157299 monohydrate, CAS No. 700874-72-2), which is an antagonist for the TGFβ receptor I kinase/ALK5 that mediates signaling of all three TGFβ isoforms.

Perivascular (infiltration): The prefix “peri-” means “around” “surrounding” or “near,” hence “perivascular” literally translates to around the blood vessels. As used herein in the context of tumor cell infiltrates, the term “perivascular infiltration” refers to a mode of entry for tumor-infiltrating immune cells (e.g., lymphocytes) via the vasculature of a solid tumor.

Potency: The term “potency” as used herein refers to activity of a drug, such as an inhibitory antibody (or fragment) having inhibitory activity, with respect to concentration or amount of the drug to produce a defined effect. For example, an antibody capable of producing certain effects at a given dosage is more potent than another antibody that requires twice the amount (dosage) to produce equivalent effects. Potency may be measured in cell-based assays, such as TGFβ activation/inhibition assays, whereby the degree of TGFβ activation, such as activation triggered by integrin binding, can be measured in the presence or absence of test article (e.g., inhibitory antibodies) in a cell-based system. Typically, among those capable of binding to the same or overlapping binding regions of an antigen (e.g., cross-blocking antibodies), antibodies with higher affinities (lower K_{D} values) tend to show higher potency than antibodies with lower affinities (greater K_{D} values).

Preclinical model: The term “preclinical model” refers to a cell line or an animal that exhibits certain characteristics of a human disease which is used to study the mechanism of action, efficacy, pharmacology, and toxicology of a drug, procedure, or treatment before it is tested on humans. Typically, cell-based preclinical studies are referred to as “in vitro” studies, whereas animal-based preclinical studies are referred to as “in vivo” studies. For example, in vivo mouse preclinical models encompassed by the current disclosure include the MBT2 bladder cancer model, the Cloudman S91 melanoma model, and the EMT6 breast cancer model.

Predictive biomarker Predictive biomarkers provide information on the probability or likelihood of response to a particular therapy. Typically, a predictive biomarker is measured before and after treatment, and the changes or relative levels of the marker in samples collected from the subject indicates or predicts therapeutic benefit.

Presenting molecule: Presenting molecules in the context of the present disclosure refer to proteins that form covalent bonds with latent pro-proteins (e.g., proTGFβ1) and tether (“present”) the inactive complex to an extracellular niche (such as ECM or immune cell surface) thereby maintaining its latency until an activation event occurs. Known presenting molecules for proTGFβ1 include: LTBP1, LTBP3, GARP (also known as LRRC32) and LRRC33, each of which can form a presenting molecule-proTGFβ1 complex (i.e., LLC), namely, LTBP1-proTGFβ1, LTBP3-proTGFβ1, GARP-proTGFβ1 and LRRC33-proTGFβ1, respectively. In nature, LTBP1 and LTBP3 are components of the extracellular matrix (ECM); therefore, LTBP1-proTGFβ1 and LTBP3-proTGFβ1 may be collectively referred to as “ECM-associated” (or “matrix-associated”) proTGFβ1 complexes, that mediate ECM-associated TGFβ1 signaling/activities. GARP and LRRC33, on the other hand, are transmembrane proteins expressed on cell surface of certain cells; therefore, GARP-proTGFβ1 and LRRC33-proTGFβ1 may be collectively referred to as “cell-associated” (or “cell-surface”) proTGFβ1 complexes, that mediate cell-associated (e.g., immune cell-associated) TGFβ1 signaling/activities.

Protection (from solvent exposure): In the context of HDX-MS-based assessment of protein-protein interactions, such as antibody-antigen binding, the degree by which a protein (e.g., a region of a protein containing an epitope) is exposed to a solvent, thereby allowing proton exchange to occur, inversely correlates with the degree of binding/interaction. Therefore, when an antibody described herein binds to a region of an antigen, the binding region is “protected” from being exposed to the solvent because the protein-protein interaction precludes the binding region from being accessible by the surrounding solvent. Thus, the protected region is indicative of a site of interaction. Typically, suitable solvents are physiological buffers.

ProTGFβ1: The term “proTGFβ1” as used herein is intended to encompass precursor forms of inactive TGFβ1 complex that comprises a prodomain sequence of TGFβ1 within the complex. Thus, the term can include the pro-, as well as the latent-forms of TGFβ1. The expression “pro/latent TGFβ1” may be used interchangeably. The “pro” form of TGFβ1 exists prior to proteolytic cleavage at the furin site. Once cleaved, the resulting form is said to be the “latent” form of TGFβ1. The “latent” complex remains non-covalently associated until further activation trigger, such as integrin-driven activation event. The proTGFβ1 complex is comprised of dimeric TGFβ1 pro-protein polypeptides, linked with disulfide bonds. The latent dimer complex is covalently linked to a single presenting molecule via the cysteine residue at position 4 (Cys4) of each of the proTGFβ1 polypeptides. The adjective “latent” may be used generally/broadly to describe the “inactive” state of TGFβ1, prior to integrin-mediated or other activation events. The proTGFβ1 polypeptide contains a prodomain (LAP) and a growth factor domain (SEQ ID NO: 1119).

Regression: Regression of tumor or tumor growth can be used as an in vivo efficacy measure. For example, in preclinical settings, median tumor volume (MTV) and Criteria for Regression Responses Treatment efficacy may be determined from the tumor volumes of animals remaining in the study on the last day. Treatment efficacy may also be determined from the incidence and magnitude of regression responses observed during the study. Treatment may cause partial regression (PR) or complete regression (CR) of the tumor in an animal. Complete regression achieved in response to therapy (e.g., administration of a drug) may be referred to as “complete response” and the subject that achieves complete response may be referred to as a “complete responder”. Thus, complete response excludes spontaneous complete regression. In some embodiments of preclinical tumor models, a PR response is defined as the tumor volume that is 50% or less of its Day 1 volume for three consecutive measurements during the course of the study, and equal to or greater than 13.5 mm³ for one or more of these three measurements. In some embodiments, a CR response is defined as the tumor volume that is less than 13.5 mm³ for three consecutive measurements during the course of the study. In preclinical model, an animal with a CR response at the termination of a study may be additionally classified as a tumor-free survivor (TFS). The term “effective tumor control” may be used to refer to a degree of tumor regression achieved in response to treatment, where, for example, the tumor volume is reduced to <25% of the endpoint tumor volume in response to treatment. For instance, in a particular model, if the endpoint tumor volume is 2,000 mm³, effective tumor control is achieved if the tumor is reduced to less than 500 mm³. Therefore, effective tumor control encompasses complete regression, as well as partial regression that reaches the threshold reduction. Similarly, regression of fibrosis can be used as an in vivo efficacy measure of a therapy such as a TGFβ1 inhibitor. The regression of fibrotic conditions may be determined based on the standard criteria to assess the severity of fibrotic manifestation by disease stage.

Regulatory T cells: “Regulatory T cells,” or Tregs, are a type of immune cells characterized by the expression of the biomarkers CD4, FOXP3, and CD25. Tregs are sometimes referred to as suppressor T cells and represent a subpopulation of T cells that modulate the immune system, maintain tolerance to self-antigens, and prevent autoimmune disease. Tregs are immunosuppressive and generally suppress or downregulate induction and proliferation of effector T (Teff) cells. Tregs can develop in the thymus (so-called CD4+ Foxp3+ “natural” Tregs) or differentiate from naïve CD4+ T cells in the periphery, for example, following exposure to TGFβ or retinoic acid. Tregs can express cell surface GARP-proTGFβ1.

Resistance (to therapy): Resistance to a particular therapy (such as CBT) may be due to the innate characteristics of the disease such as cancer (“primary resistance”, i.e., present before treatment initiation), or due to acquired phenotypes that develop over time following the treatment (“acquired resistance”). Patients who do not show therapeutic response to a therapy (e.g., those who are non-responders or poorly responsive to the therapy) are said to have primary resistance or acquired resistance to the therapy and may be characterized as primary non-responders. Patients who have never previously received a treatment and do not show a therapeutic response to the treatment are said to have primary resistance. Patients who initially show therapeutic response to a therapy but later lose effects (e.g., progression or recurrence despite continued therapy) are said to have acquired resistance to the therapy. In the context of immunotherapy, such resistance can indicate immune escape.

Response Evaluation Criteria in Solid Tumors (RECIST) and iRECIST: RECIST is a set of published rules that define when tumors in cancer patients improve (“respond”), stay the same (“stabilize”), or worsen (“progress”) during treatment. The criteria were published in February 2000 by an international collaboration including the European Organisation for Research and Treatment of Cancer (EORTC), National Cancer Institute of the United States, and the National Cancer Institute of Canada Clinical Trials Group. Subsequently, a revised version of the RECIST guideline (RECIST v 1.1) has been widely adapted (see: Eisenhauera et al., (2009), “New response evaluation criteria in solid tumours: Revised RECIST guideline (version 1.1)” Eur J Cancer 45: 228-247, incorporated herein).

Response criteria are as follows: Complete response (CR): Disappearance of all target lesions; Partial response (PR): At least a 30% decrease in the sum of the LD of target lesions, taking as reference the baseline sum LD; Stable disease (SD): Neither sufficient shrinkage to qualify for PR nor sufficient increase to qualify for PD, taking as reference the smallest sum LD since the treatment started; Progressive disease (PD): At least a 20% increase in the sum of the LD of target lesions, taking as reference the smallest sum LD recorded since the treatment started or the appearance of one or more new lesions.

On the other hand, iRECIST provides a modified set of criteria that takes into account immune-related response (see: ncbi.nlm.nih.gov/pmc/articles/PMC5648544/, Seymour et al., iRECIST: guidelines for response criteria for use in trials testing immunotherapeutics, Lancet Oncol., 2017, the contents of which are incorporated herein by reference). The RECIST and iRECIST criteria are standardized, may be revised from time to time as more data become available, and are well understood in the art.

Response rate: The term response rate (as in “low response rates”) as used herein carries the ordinary meaning as understood by the skilled person in medicine, such as oncologists. A response rate is the proportion (e.g., fraction or percentage) of subjects in a patient population who shows clinical improvement upon receiving a treatment (e.g., pharmacological intervention) and may include complete response and partial response. In oncology, clinical improvement may include tumor shrinkage (e.g., partial response) or disappearance (e.g., complete response). When used as a clinical endpoint for clinical trials of cancer treatments, this is typically expressed as the objective response rate (ORR). The FDA defines ORR as the proportion of patients with tumor size reduction of a predefined amount and for a minimum time period. See: “Clinical Trial Endpoints for the Approval of Cander Drugs and Biologics—Guidance for Industry” published by U.S. Department of Health and Human Services, Food and Drug Administration, Oncology Center of Excellence, Center for Drug Evaluation and Research (CDER), Center for Biologics Evaluation and Research (CBER), the contents of which is incorporated herein by reference.

Solid tumor The term “solid tumor” refers to proliferative disorders resulting in an abnormal growth or mass of tissue that usually does not contain cysts or liquid areas. Solid tumors may be benign (non-cancerous), or malignant (cancerous). Solid tumors include tumors of advanced malignancies, such as locally advanced solid tumors and metastatic cancer. Solid tumors are typically comprised of multiple cell types, including, without limitation, cancerous (malignant) cells, stromal cells such as CAFs, and infiltrating leukocytes, such as macrophages, MDSCs and lymphocytes. Solid tumors to be treated with an isoform-selective inhibitor of TGFβ1, such as those described herein, are typically TGFβ1-positive (TGFβ1+) tumors, which may include multiple cell types that produce TGFβ1. In certain embodiments, the TGFβ1+ tumor may also co-express TGFβ3 (i.e., TGFβ3-positive). For example, certain tumors are TGFβ1/3-co-dominant. In some embodiments, such tumors are caused by cancer of epithelial cells, e.g., carcinoma. In certain embodiments, such tumors include ovarian cancer, breast cancer, bladder cancer, pancreatic cancer, e.g., pancreatic adenocarcinoma, prostate cancer, e.g., prostate adenocarcinoma, melanoma, e.g., skin cutaneous melanoma, lung cancer, e.g., lung squamous cell carcinoma and lung adenocarcinoma, liver cancer (e.g., liver hepatocellular carcinoma), uterine cancer, e.g., uterine corpus endometrial carcinoma, kidney cancer, e.g., renal clear cell carcinoma, head and neck cancer, e.g., head and neck squamous cell carcinoma, colon cancer, e.g., colon adenocarcinoma, esophageal carcinoma, and tenosynovial giant cell tumor (TGCT). In some embodiments, a solid tumor treated herein, such as one or more of those listed above, exhibits elevated TGFβ1 expression as compared to other tumor types and exhibits a reduced responsiveness to mainline therapy, e.g., genotoxic therapy. Accordingly, TGFβ inhibitors (e.g., Ab6) may be used in conjunction with one or more genotoxic therapies (e.g., chemotherapy and/or radiation therapy, including radiotherapeutic agents) to treat such cancer in a subject.

Solution Equilibrium Titration (SET): The SET is an assay whereby binding between two molecules (such as an antigen and an antibody that binds the antigen) can be measured at equilibrium in a solution. For example, Meso-Scale Discovery (“MSD”)-based SET, or MSD-SET, is a useful mode of determining dissociation constants for particularly high-affinity protein-protein interactions at equilibrium, such as picomolar-affinity antibodies binding to their antigens (see, for example: Ducata et al., (2015) J Biomolecular Screening 20(10): 1256-1267). The SET-based assays are particularly useful for determining KD values of antibodies with sub-nanomolar (e.g., picomolar) affinities.

Specific binding: As used herein, the term “specific binding” or “specifically binds” means that an antibody, or antigen binding portion thereof, exhibits a particular affinity for a particular structure (e.g., an antigenic determinant or epitope) in an antigen (e.g., a K_{D} measured by Biacore®). For example, the antibody, or antigen-binding portion thereof, binds to a specific protein rather than to proteins generally. In some embodiments, an antibody, or antigen binding portion thereof, specifically binds to a target, e.g., TGFβ1, if the antibody has a K_{D} for the target of at least about 10⁻⁸ M, 10⁻⁹ M, 10⁻¹⁰ M, 10⁻¹¹ M, 10⁻¹² M, or less. In some embodiments, the term “specific binding to an epitope of proTGFβ1”, “specifically binds to an epitope of proTGFβ1”, “specific binding to proTGFβ1”, or “specifically binds to proTGFβ1” as used herein, refers to an antibody, or antigen binding portion thereof, that binds to proTGFβ1 and has a dissociation constant (K_{D}) of 1.0×10⁻⁸ M or less, as determined by suitable in vitro binding assays, such as surface plasmon resonance and Biolayer Interferometry (BLI). In preferred embodiments, kinetic rate constants (e.g., K_{D}) are determined by surface plasmon resonance (e.g., a Biacore system). In one embodiment, an antibody, or antigen binding portion thereof, can specifically bind to both human and a non-human (e.g., mouse) orthologues of proTGFβ1. In some embodiments, an antibody may also “selectively” (i.e., “preferentially”) bind a target antigen if it binds that target with a comparatively greater strength than the strength of binding shown to other antigens, e.g., a 10-fold, 100-fold, 1000-fold, or greater comparative affinity for a target antigen (e.g., TGFβ1) than for a non-target antigen (e.g., TGFβ2 and/or TGFβ3). In preferred embodiments, an isoform-selective inhibitor exhibits no detectable binding or potency towards other isoforms or counterparts. In some embodiments, an antibody that binds specifically to a set of antigens may have high affinity toward said antigens but may not distinguish said antigens from one another (i.e., the antibody is specific but not selective). In some embodiments, an antibody that binds to an antigen with a particularly high affinity as compared to other antigens may be considered selective for said antigen. For instance, an antibody that binds to antigen X with 1000-fold higher affinity as compared to antigen Y may be considered an antibody that is selective for antigen X over antigen Y. In the context of the present disclosure, “an antibody that specifically binds an antigen with high affinity” generally refers to a K_{D} of 1.0×10−8 M or less.

Subject: The term “subject” in the context of therapeutic applications refers to an individual who receives or is in need of clinical care or intervention, such as treatment, diagnosis, etc. Suitable subjects include vertebrates, including but not limited to mammals (e.g., human and non-human mammals). Where the subject is a human subject, the term “patient” may be used interchangeably. In a clinical context, the term “a patient population” or “patient subpopulation” is used to refer to a group of individuals that falls within a set of criteria, such as clinical criteria (e.g., disease presentations, disease stages, susceptibility to certain conditions, responsiveness to therapy, etc.), medical history, health status, gender, age group, genetic criteria (e.g., carrier of certain mutation, polymorphism, gene duplications, DNA sequence repeats, etc.) and lifestyle factors (e.g., smoking, alcohol consumption, exercise, etc.).

Surface plasmon resonance (SPR): Surface plasmon resonance is an optical phenomenon that enables detection of unlabeled interactants in real time. The SPR-based biosensors, such as those commercially available from Biacore, can be employed to measure biomolecular interactions, including protein-protein interactions, such as antigen-antibody binding. The technology is widely known in the art and is useful for the determination of parameters such as binding affinities, kinetic rate constants and thermodynamics.

Target engagement: As used herein, the term target engagement refers to the ability of a molecule (e.g., TGFβ inhibitor) to bind to its intended target in vivo (e.g., endogenous TGFβ). In case of activation inhibitors, the intended target can be a large latent complex.

TGFβ1-related indication: A “TGFβ1-related indication” is a TGFβ1-associated disorder and means any disease or disorder, and/or condition, in which at least part of the pathogenesis and/or progression is attributable to TGFβ1 signaling or dysregulation thereof. Certain TGFβ1-associated disorders are driven predominantly by the TGFβ1 isoform. Subjects having a TGFβ1-related indication may benefit from inhibition of the activity and/or levels TGFβ1. Certain TGFβ1-related indications are driven predominantly by the TGFβ1 isoform. TGFβ1-related indications include, but are not limited to: fibrotic conditions (such as organ fibrosis, and fibrosis of tissues involving chronic inflammation), proliferative disorders (such as cancer, e.g., solid tumors and myelofibrosis), disease associated with ECM dysregulation (such as conditions involving matrix stiffening and remodeling), disease involving mesenchymal transition (e.g., EndMT and/or EMT), disease involving proteases, disease with aberrant gene expression of certain markers described herein. These disease categories are not intended to be mutually exclusive. According to some embodiments, the TGFβ1-related indication is fibrosis, e.g., lung fibrosis.

TGFβ inhibitor The term “TGFβ inhibitor” refers to any agent capable of antagonizing biological activities, signaling or function of TGFβ growth factor (e.g., TGFβ1, TGFβ2 and/or TGFβ3). The term is not intended to limit its mechanism of action and includes, for example, neutralizing inhibitors, receptor antagonists, soluble ligand traps, TGFβ activation inhibitors, and integrin inhibitors (e.g., antibodies that bind to αVβ1, αVβ3, αVβ5, αVβ6, αVβ8, a5β1, αIIbβ3, or α8β1 integrins, and inhibit downstream activation of TGFβ. e.g., selective inhibition of TGFβ1 and/or TGFβ3). The term encompasses TGFβ inhibitors that are isoform-selective and non-selective inhibitors. The latter, commonly referred to as “pan-inhibitors” of TGFβ, include, for example, small molecule receptor kinase inhibitors (e.g., ALK5 inhibitors), antibodies (such as neutralizing antibodies) that preferentially bind two or more isoforms, and engineered constructs (e.g., fusion proteins) comprising a ligand-binding moiety. In certain embodiments, these antibodies may include or may be engineered to include a mutation or modification that causes an extended half-life of the antibody. In some embodiments, such mutations or modifications may be within the Fc domain of the antibodies (e.g., Fc-modified antibodies). In some embodiments, the mutation is so-called YTE mutation. TGFβ inhibitors also include antibodies that are capable of reducing the availability of latent proTGFβwhich can be activated in the niche, for example, by inducing antibody-dependent cell mediated cytotoxicity (ADCC), and/or antibody-dependent cellular phagocytosis (ADPC), as well as antibodies that result in internalization of cell-surface complex comprising latent proTGFβ, thereby removing the precursor from the plasma membrane without depleting the cells themselves. Internalization may be a suitable mechanism of action for LRRC33-containing protein complexes (such as human LRRC33-proTGFβ1) which results in reduced levels of cells expressing LRRC33-containing protein complexes on cell surface.

The “TGFβ family” is a class within the TGFβ superfamily and in human contains three members: TGFβ1, TGFβ2, and TGFβ3, which are structurally similar. The three growth factors are known to signal via the same receptors.

TGFβ1-positive cancer/tumor: The term, as used herein, refers to a cancer/tumor with aberrant TGFβ1 expression (overexpression). Many human cancer/tumor types show predominant expression of the TGFβ1 (note that “TGFB” is sometimes used to refer to the gene as opposed to protein) isoform. In some cases, such cancer/tumor may show co-dominant expression of another isoform, such as TGFβ3. A number of epithelial cancers (e.g., carcinoma) may co-express TGFβ1 and TGFβ3. Within the tumor environment of TGFβ1-positive tumors, TGFβ1 may arise from multiple sources, including, for example, cancer cells, tumor-associated macrophages (TAMs), cancer-associated fibroblasts (CAFs), regulatory T cells (Tregs), myeloid-derived suppressor cells (MDSCs), and the surrounding extracellular matrix (ECM). In the context of the present disclosure, preclinical cancer/tumor models that recapitulate human conditions are TGFβ1-positive cancer/tumor.

Therapeutic window: The term “therapeutic window” refers to a dosage/concentration range that produces therapeutic response without causing significant/observable/unacceptable adverse effect (e.g., within adverse effects that are acceptable or tolerable) in subjects. Therapeutic window may be calculated as a ratio between minimum effective concentrations (MEC) to the minimum toxic concentrations (MTC). To illustrate, a TGFβ1 inhibitor that achieves in vivo efficacy at 10 mg/kg dosage and shows tolerability or acceptable toxicities at 100 mg/kg provides at least a 10-fold (e.g., 10×) therapeutic window. By contrast, a pan-inhibitor of TGFβ that is efficacious at 10 mg/kg but causes adverse effects at less than the effective dose (e.g., at 5 mg/kg) is said to have “dose-limiting toxicities.” Generally, the maximally tolerated dose (MTD) may set the upper limit of the therapeutic window. For example, Ab6 was shown to be efficacious at dosage ranging between about 3-30 mg/kg/week and was also shown to be free of observable toxicities associated with pan-inhibition of TGFβ at dosage of at least 100 or 300 mg/kg/week for 4 weeks in rats or non-human primates. Based on this, Ab6 shows at minimum a 3.3-fold and up to 100-fold therapeutic window. In some embodiments, the concept of therapeutic window may be expressed in terms of safety factors.

Toxicity: As used herein, the term “toxicity” or “toxicities” refers to unwanted in vivo effects in subjects (e.g., patients) associated with a therapy administered to the subjects (e.g., patients), such as undesirable side effects and adverse events. “Tolerability” refers to a level of toxicities associated with a therapy or therapeutic regimen, which can be reasonably tolerated by patients, without discontinuing the therapy due to the toxicities. Typically, toxicity/toxicology studies are carried out in one or more preclinical models prior to clinical development to assess safety profiles of a drug candidate (e.g., monoclonal antibody therapy). Toxicity/toxicology studies may help determine the “no-observed-adverse-effect level (NOAEL)” and the “maximally tolerated dose (MTD)” of a test article, based on which a therapeutic window may be deduced. Preferably, a species that is shown to be sensitive to the particular intervention should be chosen as a preclinical animal model in which safety/toxicity study is to be carried out. In case of TGFβ inhibition, suitable species include rats, dogs, and cynos. Mice are reported to be less sensitive to pharmacological inhibition of TGFβ and may not reveal toxicities that are potentially dangerous in other species, including human, although certain studies report toxicities observed with pan-inhibition of TGFβ in mice. To illustrate in the context of the present disclosure, the NOAEL for Ab6 in rats was the highest dose evaluated (100 mg/kg), suggesting that the MTD is >100 mg/kg, based on a four-week toxicology study. The MTD of Ab6 in non-human primates is >300 mg/kg based on a four-week toxicology study.

For determining NOAELs and MTDs, preferably, a species that is shown to be sensitive to the particular intervention should be chosen as a preclinical animal model in which safety/toxicology study is to be carried out. In case of TGFβ inhibition, suitable species include, but are not limited to, rats, dogs, and cynos. Mice are reported to be less sensitive to pharmacological inhibition of TGFβ and may not reveal toxicities that are potentially serious or dangerous in other species, including human.

Translatability: In the context of drug discovery and clinical development, the term “translatability” or “translatable” refers to certain quality or property of preclinical models or data that recapitulate human conditions. As used herein, a preclinical model that recapitulates a TGFβ1 indication typically shows predominant expression of TGFB1 (or TGFβ1), relative to TGFB2 (or TGFβ2) and TGFB3 (or TGFβ3). In combination therapy paradigms, for example, translatability may require the same underlining mechanisms of action that the combination of actives is aimed to effectuate in the model. As an example, many human tumors are immune excluded, TGFβ1-positive tumors that show primary resistance to a checkpoint blockade therapy (CBT). A second therapy (such as TGFβ1 inhibitors) may be used in combination to overcome the resistance to CBT. In this scenario, suitable translatable preclinical models include TGFβ1-positive tumors that show primary resistance to a checkpoint blockade therapy (CBT).

Treat/treatment: The term “treat” or “treatment” includes therapeutic treatments, prophylactic treatments, and applications in which one reduces the risk that a subject will develop a disorder or other risk factor. Thus the term is intended to broadly mean: causing therapeutic benefits in a patient by, for example, slowing disease progression, reversing certain disease features, normalizing gene expression, or boosting the body's immunity; reducing or reversing immune suppression; reducing, removing or eradicating harmful cells or substances from the body; reducing disease burden (e.g., fibrosis and tumor burden); preventing recurrence or relapse; prolonging a refractory period, and/or otherwise improving survival. The term includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In the context of combination therapy, the term may also refer to: i) the ability of a second therapeutic to reduce the effective dosage of a first therapeutic so as to reduce side effects and increase tolerability; ii) the ability of a second therapy to render the patient more responsive to a first therapy; and/or iii) the ability to effectuate additive or synergistic clinical benefits.

Tumor-associated macrophage (TAM): TAMs are polarized/activated macrophages with pro-tumor phenotypes (M2-like macrophages). TAMs can be either marrow-originated monocytes/macrophages recruited to the tumor site or tissue-resident macrophages which are derived from erythro-myeloid progenitors. Differentiation of monocytes/macrophages into TAMs is influenced by a number of factors, including local chemical signals such as cytokines, chemokines, growth factors and other molecules that act as ligands, as well as cell-cell interactions between the monocytes/macrophages that are present in the niche (tumor microenvironment). Generally, monocytes/macrophages can be polarized into so-called M1” or “M2” subtypes, the latter being associated with more pro-tumor phenotype. In a solid tumor, up to 50% of the tumor mass may correspond to macrophages, which are preferentially M2-polarized. Among tumor-associated monocytes and myeloid cell populations, M1 macrophages typically express cell surface HLA-DR, CD68 and CD86, while M2 macrophages typically express cell surface HLA-DR, CD68, CD163 and CD206. Tumor-associated, M2-like macrophages (such as M2c and M2d subtypes) can express cell surface LRRC33 and/or LRRC33-proTGFβ1. M2-like macrophages may be also enriched in fibrotic microenvironment.

Tumor microenvironment: The term “tumor microenvironment (TME)” refers to a local disease niche, in which a tumor (e.g., solid tumor) resides in vivo. The TME may comprise disease-associated molecular signature (a set of chemokines, cytokines, etc.), disease-associated cell populations (such as TAMs, CAFs, MDSCs, etc.) as well as disease-associated ECM environments (alterations in ECM components and/or structure).

Valvulopathy: The term “valvulopathy” refers to a disease, disorder, or condition affecting one or more of the four valves of the heart, often characterized by lesions on the valve(s) of the heart. It is also generally known as valvular heart disease, or cardiac valvulopathy. Types of valvulopathies include, but are not limited to, aortic valvulopathies (e.g., aortic stenosis), mitral valvulopathies, tricuspid valvulopathies, and pulmonary valvulopathies.

Variable region: The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

Other than in the operating examples, or where otherwise indicated, all numbers expressing quantities of ingredients or reaction conditions used herein should be understood as modified in all instances by the term “about.” The term “about” means±10% of the recited value.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, e.g., 10-20, 1-10, 30-40, etc.

### Transforming Growth Factor-beta (TGFβ)

The Transforming Growth Factor-beta (TGFβ) activities and subsequent partial purification of the soluble growth factors were first described in the late 1970's to early 1980's, with which the TGFβ field began some 40 years ago. To date, 33 gene products have been identified that make up the large TGFβ superfamily. The TGFβ superfamily can be categorized into at least three subclasses by structural similarities: TGFβ s, Growth-Differentiation Factors (GDFs) and Bone-Morphogenetic Proteins (BMPs). The TGFβ subclass is comprised of three highly conserved isoforms, namely, TGFβ1, TGFβ2 and TGFβ3, which are encoded by three separate genes in human.

TABLE 1
Select protein domains/motifs of human TGFβ1-related polypeptides

| Human TGFβ1 | | SEQ ID |
|---|---|---|
| domain/module | Amino Acid Sequence | NO |
| Latency Associated | LSTCKTIDMELVKRKRIEAIRGQILSKLRLASPPSQGEVPPGPLPEAVLA | 1119 |
| Peptide (LAP) | LYNSTRDRVAGESAEPEPEPEADYYAKEVTRVLMVETHNEIYDKFKQS | |
| (prodomain) | THSIYMFFNTSELREAVPEPVLLSRAELRLLRLKLKVEQHVELYQKYSN | |
| | NSWRYLSNRLLAPSDSPEWLSFDVTGVVRQWLSRGGEIEGFRLSAHC | |
| | SCDSRDNTLQVDINGFTTGRRGDLATIHGMNRPFLLLMATPLERAQHL | |
| | QSSRHRR | |
| | (“First binding region” is underlined) | |
| Straight Jacket | LSTCKTIDMELVKRKRIEAIRGQILSKLRLASPPSQGEVPPGPLP | 1120 |
| | (“Latency Lasso” is underlined) | |
| Growth Factor | ALDTNYCFSSTEKNCCVRQLYIDFRKDLGWKWIHEPKGYHANFCLGP | 1121 |
| Domain | CPYIWSLDTQYSKVLALYNQHNPGASAAPCCVPQALEPLPIVYYVGRK | |
| | PKVEQLSNMIVRSCKCS | |
| | (“Finger-1” and “Finger-2” are underlined, | |
| | respectively) | |
| Fastener | residues 74-76, YYA | n/a |
| Furin cleavage site | RHRR | 1122 |
| Arm | EAVLALYNSTRDRVAGESAEPEPEPEADYYAKEVTRVLMVETHNEIYD | 1123 |
| | KFKQSTHSIYMFFNTSELREAVPEPVLLSRAELRLLRLKLKVEQHVELY | |
| | QKYSNNSWRYLSNRLLAPSDSPEWLSFDVTGVVRQWLSRGGEIEGFR | |
| | LSAHCSCDSRDNTLQVDINGFTTGRRGDLATIHGMNRPFLLLMATPLE | |
| | RAQHLQSSRHRR | |
| Finger-1 | CVRQLYIDFRKDLGWKWIHEPKGYHANFC | 1124 |
| | (“Second binding region” is underlined) | |
| Finger-2 | CVPQALEPLPIVYYVGRKPKVEQLSNMIVRSCKCS | 1125 |
| | (“Third binding region” is underlined) | |
| Residue for | Cys 4 | n/a |
| presenting | | |
| molecule | | |
| association | | |
| Latency Lasso | LASPPSQGEVPPGPL | 1126 |
| | (Portion of the binding regions | |
| | shared across 4 different isoform- | |
| | selective proTGFβ1 antibodies is underlined) | |
| Extended Latency | LASPPSQGEVPPGPLPEAVLALYNSTR | 1127 |
| Lasso | (Portion of the binding regions | |
| | shared across 4 different isoform- | |
| | selective proTGFβ1 antibodies is underlined) | |
| Alpha-1 Helix | LSTCKTIDMELVKRKRIEAIRGQILSKLR | 1128 |
| Alpha-2 Helix | AVLALYNSTR | 1129 |
| Trigger Loop | NGFTTGRRGDLATIHGMNRP | 1130 |
| Integrin binding | residue 215-217, RGD | n/a |
| Bowtie | CSCDSRDNTLQVD | 1131 |

The TGFβ s are thought to play key roles in diverse processes, such as inhibition of cell proliferation, extracellular matrix (ECM) remodeling, and immune homeostasis. The importance of TGFβ1 for T cell homeostasis is demonstrated by the observation that TGFβ1−/− mice survive only 3-4 weeks, succumbing to multi-organ failure due to massive immune activation (Kulkarni, A. B., et al., Proc Natl Acad Sci USA, 1993. 90(2): p. 770-4; Shull, M. M., et al., Nature, 1992. 359(6397): p. 693-9). The roles of TGFβ2 and TGFβ3 are less clear. Whilst the three TGFβ isoforms have distinct temporal and spatial expression patterns, they signal through the same receptors, TGFβ RI and TGFβ RII, although in some cases, for example for TGFβ2 signaling, type III receptors such as betaglycan are also required (Feng, X. H. and R. Derynck, Annu Rev Cell Dev Biol, 2005. 21: p. 659-93; Massague, J., Annu Rev Biochem, 1998. 67: p. 753-91). Ligand-induced oligomerization of TGFβ RI/II triggers the phosphorylation of SMAD transcription factors, resulting in the transcription of target genes, such as Col1a1, Col3a1, ACTA2, and SERPINE1 (Massague, J., J. Seoane, and D. Wotton, Genes Dev, 2005. 19(23): p. 2783-810). SMAD-independent TGFβ signaling pathways have also been described, for example in cancer or in the aortic lesions of Marfan mice (Derynck, R. and Y. E. Zhang, Nature, 2003. 425(6958): p. 577-84; Holm, T. M., et al., Science, 2011. 332(6027): p. 358-61).

The biological importance of the TGFβ pathway in humans has been validated by genetic diseases. Camurati-Engelman disease results in bone dysplasia due to an autosomal dominant mutation in the TGFB1 gene, leading to constitutive activation of TGFβ1 signaling (Janssens, K., et al., J Med Genet, 2006. 43(1): p. 1-11). Patients with Loeys/Dietz syndrome carry autosomal dominant mutations in components of the TGFβ signaling pathway, which cause aortic aneurism, hypertelorism, and bifid uvula (Van Laer, L., H. Dietz, and B. Loeys, Adv Exp Med Biol, 2014. 802: p. 95-105). As TGFβ pathway dysregulation has been implicated in multiple diseases, several drugs that target the TGFβ pathway have been developed and tested in patients, but with limited success.

Dysregulation of the TGFβ signaling has been associated with a wide range of human diseases. Indeed, in a number of disease conditions, such dysregulation may involve multiple facets of TGFβ function. Diseased tissue, such as fibrotic and/or inflamed tissues and tumors, may create a local environment in which TGFβ activation can cause exacerbation or progression of the disease, which may be at least in part mediated by interactions between multiple TGFβ-responsive cells, which are activated in an autocrine and/or paracrine fashion, together with a number of other cytokines, chemokines and growth factors that play a role in a particular disease setting.

For example, a tumor microenvironment (TME) contains multiple cell types expressing TGFβ1, such as activated myofibroblast-like fibroblasts, stromal cells, infiltrating macrophages, MDSCs and other immune cells, in addition to cancer (i.e., malignant) cells. Thus, the TME represents a heterogeneous population of cells expressing and/or responsive to TGFβ1 but in association with more than one types of presenting molecules, e.g., LTBP1, LTBP3, LRRC33 and GARP, within the niche.

Advances in immunotherapy have transformed the effective treatment landscape for a growing number of cancer patients. Most prominent are the checkpoint blockade therapies (CBT), which have now become part of standard of care regimens for an increasing number of cancers. While profound and durable responses to CBT have been observed across a growing number of cancer types, it is now clear that a significant fraction of tumors appear to be refractory to CBT even at the outset of treatment, hence pointing to primary resistance as a major challenge to enabling many patients' immune systems to target and eliminate tumor cells. Efforts to understand and address the underlying mechanisms conferring primary resistance to CBT have been undertaken in order to broaden treatment efficacy for a greater number of patients. However, this enthusiasm has been curbed by lackluster clinical trial results and failures when combining CBTs with agents known to affect the same tumor type or to modulate seemingly relevant components of the immune system. A likely reason is that a clear mechanistic rationale for the given combination is often not rooted in clinically-derived data, and has thus led to uncertain and confounding outcomes in trials intended to enhance approved single-agent therapies. It has become clear that the design of combination immunotherapy should be rooted in scientific evidence of relevance to underlying tumor and immune system biology.

Recently, a phenomenon referred to as “immune exclusion” was coined to describe a tumor environment from which anti-tumor effector T cells (e.g., CD8+ T cells) are kept away (hence “excluded”) by immunosuppressive local cues. More recently, a number of retrospective analyses of clinically-derived tumors have implicated TGFβ pathway activation in mediating primary resistance to CBT. For example, transcriptional profiling and analysis of pretreatment melanoma biopsies revealed an enrichment of TGFβ-associated pathways and biological processes in tumors that are non-responsive to anti-PD-1 CBT. In an immune-excluded tumor, effector cells, which would otherwise be capable of attacking cancer cells by recognizing cell-surface tumor antigens, are prevented from gaining access to the site of cancer cells. In this way, cancer cells evade host immunity and immuno-oncologic therapeutics, such as checkpoint inhibitors, that exploit and rely on such immunity. Indeed, such tumors show resistance to checkpoint inhibition, such as anti-PD-1 and anti-PD-L1 antibodies, presumably because target T cells are blocked from entering the tumor hence failing to exert anti-cancer effects.

A number of retrospective analyses of clinically-derived tumors points to TGFβ pathway activation in mediating primary resistance to CBT. For example, transcriptional profiling and analysis of pretreatment melanoma biopsies revealed an enrichment of TGFβ-associated pathways and biological processes in tumors that are non-responsive to anti-PD-1 CBT. More recently, similar analyses of tumors from metastatic urothelial cancer patients revealed that lack of response to PD-L1 blockade with atezolizumab was associated with transcriptional signatures of TGFβ signaling, particularly in tumors wherein CD8+ T cells appear to be excluded from entry into the tumor. The critical role of TGFβ signaling in mediating immune exclusion resulting in anti-PD-(L)1 resistance has been verified in the EMT-6 syngeneic mouse model of breast cancer. While the EMT-6 tumors are weakly responsive to treatment with an anti-PD-L1 antibody, combining this checkpoint inhibitor with 1D11, an antibody that blocks the activity of all TGFβ isoforms, resulted in a profound increase in the frequency of complete responses when compared to treatment with individual inhibitors. The synergistic antitumor activity is proposed to be due to a change in cancer-associated fibroblast (CAF) phenotype and a breakdown of the immune excluded phenotype, resulting in infiltration of activated CD8+ T cells into the tumors. Similar results were found in a murine model of colorectal cancer and metastasis using a combination of an anti-PD-L1 antibody with galunisertib, a small molecule inhibitor of the type I TGFβ receptor ALK5 kinase. Collectively, these findings suggest that inhibiting the TGFβ pathway in CBT-resistant tumors could be a promising approach to improve or increase the number of clinical responses to CBT. While recent work has implicated a relationship between TGFβ pathway activation and primary CBT resistance, TGFβ signaling has long been linked to features of cancer pathogenesis. As a potent immunosuppressive factor, TGFβ prevents antitumor T cell activity and promotes immunosuppressive macrophages. Malignant cells often become resistant to TGFβ signaling as a mechanism to evade its growth and tumor-suppressive effects. TGFβ activates CAFs, inducing extracellular matrix production and promotion of tumor progression. Finally, TGFβ induces EMT, thus supporting tissue invasion and tumor metastases.

Mammals have distinct genes that encode and express the three TGFβ growth factors, TGFβ1, TGFβ2, and TGFβ3, all of which signal through the same heteromeric TGFβ receptor complex. Despite the common signaling pathway, each TGFβ isoform appears to have distinct biological functions, as evidenced by the non-overlapping TGFβ knockout mouse phenotypes. All three TGFβ isoforms are expressed as inactive prodomain-growth factor complexes, in which the TGFβ prodomain, also called latency-associated peptide (LAP), wraps around its growth factor and holds it in a latent, non-signaling state. Furthermore, latent TGFβ is co-expressed with latent TGFβ-binding proteins and forms large latent complexes (LLCs) through disulfide linkage. Association of latent TGFβ with Latent TGFβ Binding Protein-1 (LTBP1) or LTBP3 enables tethering to extracellular matrix, whereas association to the transmembrane proteins GARP or LRRC33 enables elaboration on the surface of Tregs or macrophages, respectively. In vivo, latent TGFβ1 and latent TGFβ3 are activated by a subset of αV integrins, which bind a consensus RGD sequence on LAP, triggering a conformational change to release the growth factor. The mechanism by which latent TGFβ2 is activated is less clear as it lacks a consensus RGD motif. TGFβ1 release by proteolytic cleavage of LAP has also been implicated as an activation mechanism, but its biological relevance is less clear.

Although the pathogenic role of TGFβ activation is clear in several disease states, it is equally clear that therapeutic targeting of the TGFβ pathway has been challenging due to the pleiotropic effects that result from broad and sustained pathway inhibition. For example, a number of studies have shown that small molecule-mediated inhibition of the TGFβ type I receptor kinase ALK5 (TGFBR1) or blockade of all three highly related TGFβ growth factors with a high-affinity antibody resulted in severe cardiac valvulopathies in mice, rats and dogs. These “pan”-TGFβ approaches that block all TGFβ signaling therefore have a very narrow therapeutic window, which has proven to be an impediment to the treatment of a number of disease-relevant processes with very high unmet medical need. No TGFβ-targeting therapy has been approved to date and clinical trial results with such modalities have largely been disappointing, likely due to the use of what proved to be inefficacious dosing regimens that were required in order to accommodate safety concerns.

All references cited herein are incorporated by reference for any purpose. Where a reference and the specification conflict, the specification will control. It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

### Highly Potent TGFβ1-Selective Inhibitors

The present disclosure provides monoclonal antibodies and antigen-binding fragments thereof capable of binding each of the four known human LLCs (hLTBP1-proTGFβ1, hLTBP3-proTGFβ1, hGARP-proTGFβ1 and hLRRC33-proTGFβ1) with high affinity (e.g., below 1 nM K_{D}) and with slow dissociation rates (i.e., low k_{OFF} values), as measured for example by surface plasmon resonance (SPR), that are can be used in the treatment of fibrotic diseases and disorders, in particular in the treatment of pulmonary fibrosis. The antibodies and the antigen-binding fragments include isoform-selective inhibitors of TGFβ1. An example of such an antibody or the antigen-binding fragment thereof comprises an H-CDR1, an H-CDR2, and H-CDR3, an L-CDR1, an L-CDR2 and an L-CFR3, wherein: the H-CDR1 comprises GFTFADYA (SEQ ID NO: 276); the H-CDR2 comprises a sequence represented by the formula ISGSGX1AT, wherein optionally the X₁ is an A or K (SEQ ID NO: 277); the H-CDR3 comprises a sequence represented by the formula VSSGX₁WDX₂D, wherein optionally the X₁ is an H, D or Q, and wherein further optionally the X₂ is an F or Y (SEQ ID NO: 278); the L-CDR1 comprises QSISSY (SEQ ID NO: 279); the L-CDR2 comprises a sequence represented by the formula AASX₁X₂X₃X₄ wherein optionally the X₁ is an N, G or V; wherein further optionally the X₂ is an L, N or E; wherein further optionally the X₃ is a Q or E; and wherein further optionally the X₄ is an S or T (SEQ ID NO: 280); and, the L-CDR3 comprises a sequence represented by the formula QQTYX₁VPLT, wherein optionally the X₁ is a T or G (SEQ ID NO: 281). In preferred embodiments, the H-CDR2 comprises ISGSGAAT (SEQ ID NO: 282); the H-CDR3 comprises VSSGHWDYD (SEQ ID NO: 287); the L-CDR2 comprises AASGLES (SEQ ID NO: 284); and, the L-CDR3 comprises QQTYGVPLT (SEQ ID NO: 285). In some embodiments, the antibody or the fragment binds an epitope that comprises one or more of the following amino acid residues of the proTGFβ1 polypeptide sequence: S35, G37, E38, V39, P40, P41, G42, P43, R274, K280, H283 and K309. In some embodiments, the H-CDR1 may comprise the sequence GFTFADYA (SEQ ID NO: 276); the H-CDR2 may comprise the sequence ISGSGAAT (SEQ ID NO: 282); the H-CDR3 may comprise a sequence represented by the formula VSSGX₁WDX₂D, wherein optionally the X₁ is an H or Q, and wherein further optionally the X₂ is a Y or F (SEQ ID NO: 283); the L-CDR1 may comprise the sequence QSISSY (SEQ ID NO: 279); the L-CDR2 may comprise the sequence AASGLES (SEQ ID NO: 284); and, the L-CDR3 may comprise the sequence QQTYGVPLT (SEQ ID NO: 285). In preferred embodiments, the H-CDR3 is VSSGHWDYD (SEQ ID NO: 287). In some embodiments, the antibody or the fragment binds an epitope that comprises one or more of the following amino acid residues of the proTGFβ1 polypeptide sequence: S35, G37, E38, V39, P40, P41, G42, P43, R274, K280, H283 and K309.

The table below provides CDR sequences of useful variants.

TABLE 2
CDR sequences of exemplary antibody

| H-CDR1 | H-CDR2 | H-CDR3 | L-CDR1 | L-CDR2 | L-CDR3 |
|---|---|---|---|---|---|
| GFTFA | ISGSG | VSSGX₁ | QSIS | AASG | QQTYG |
| DYA | AAT | WDX₂D | SY | LES | VPLT |
| (SEQ ID | (SEQ ID | X₁ = H or Q | (SEQ | (SEQ | (SEQ |
| NO: | NO: | X₂ = Y or F | ID | ID | ID |
| 276) | 282) | (SEQ ID | NO: | NO: | NO: |
| | | NO: 283) | 279) | 284) | 285) |

Optionally, one or more of the six CDRs may include one or more (e.g., 1 or 2) amino acid change(s).

In some embodiments, an antibody or an antigen-binding fragment thereof selected for use or manufacture according to the present disclosure comprises an H-CDR1, an H-CDR2, and H-CDR3, an L-CDR1, an L-CDR2 and an L-CFR3, wherein: the H-CDR1 comprises GFTFADYA (SEQ ID NO: 276); the H-CDR2 comprises a sequence represented by the formula ISGSGX1AT, wherein optionally the X₁ is an A or K (SEQ ID NO: 277); the H-CDR3 comprises a sequence represented by the formula VSSGX₁WDX₂D, wherein optionally the X₁ is an H, D or Q, and wherein further optionally the X₂ is an F or Y (SEQ ID NO: 278); the L-CDR1 comprises QSISSY (SEQ ID NO: 279); the L-CDR2 comprises a sequence represented by the formula AASX₁X₂X₃X₄ wherein optionally the X₁ is an N, G or V; wherein further optionally the X₂ is an L, N or E; wherein further optionally the X₃ is a Q or E; and wherein further optionally the X₄ is an S or T (SEQ ID NO: 280); and, the L-CDR3 comprises a sequence represented by the formula QQTYX₁VPLT, wherein optionally the X₁ is a T or G (SEQ ID NO: 281). In preferred embodiments, the H-CDR2 comprises ISGSGAAT (SEQ ID NO: 282); the H-CDR3 comprises VSSGHWDYD (SEQ ID NO: 287); the L-CDR2 comprises AASGLES (SEQ ID NO: 284); and, the L-CDR3 comprises QQTYGVPLT (SEQ ID NO: 285). In some embodiments, the antibody or the fragment binds an epitope that comprises one or more of the following amino acid residues of the proTGFβ1 polypeptide sequence: S35, G37, E38, V39, P40, P41, G42, P43, R274, K280, H283 and K309.

The table below further provides CDR sequences of useful variants.

TABLE 3
CDR variants

| H-CDR1 | H-CDR2 | H-CDR3 | L-CDR1 | L-CDR2 | L-CDR3 |
|---|---|---|---|---|---|
| GFTFADYA | ISGSGX₁AT | VSSGX₁WDX₂D | QSISSY | AASX₁X₂X₃X₄ | QQTYX₁VPLT |
| (SEQ ID | wherein optionally | wherein optionally | (SEQ ID | wherein optionally | wherein |
| NO: 276) | the X₁ is A or K | X₁ is H, D or Q; | NO: 279) | X₁ is N, G or V; | optionally |
| | (SEQ ID NO: 277) | and wherein | | wherein further | X₁ is T or G |
| | | further optionally | | optionally | (SEQ ID NO: |
| | | X₂ is F or Y (SEQ | | X₂ is L, N or E; | 281) |
| | | ID NO: 278) | | wherein further | |
| | | | | optionally | |
| | | | | X₃ is Q or E; and | |
| | | | | wherein further | |
| | | | | optionally | |
| | | | | X₄ is S or T | |
| | | | | (SEQ ID NO: 280) | |

In some embodiments, one or more of the six CDRs may include one or more (e.g., 1 or 2) amino acid change.

Non-limiting examples of preferred activation inhibitors of TGFβ1 are provided in the table below, herein referred to as: Ab37, Ab38, Ab39, Ab40, Ab41, Ab43, Ab44, Ab45, Ab46, Ab47, Ab48, Ab49, Ab50, Ab51 and Ab52. Each of these antibodies may be in the form of whole immunoglobulin (such as IgG) or an antigen-binding fragment thereof, such as the Fab fragment. The antigen-binding fragment may be used to make an engineered construct that comprises the fragment or a derivative thereof, such as bispecific antibodies and other fusion proteins that functions as a TGFβ1 inhibitor. The six CDRs of each of the exemplary antibodies are listed in the table below. In some embodiments, the activation inhibitors of TGFβ1 is AB46.

TABLE 4
Exemplary CDR sequences

| | | SEQ | | SEQ | | SEQ | | SEQ | | SEQ | | SEQ |
|---|---|---|---|---|---|---|---|---|---|---|---|---|
| | | ID | | ID | | ID | | ID | | ID | | ID |
| | H-CDR1 | NO: | H-CDR2 | NO: | H-CDR3 | NO: | L-CDR1 | NO: | L-CDR2 | NO: | L-CDR3 | NO: |
| Ab37 | GFTFADYA | 276 | ISGSGAAT | 282 | VSSGHWDFD | 289 | QSISSY | 279 | AASNLES | 292 | QQTYTVPLT | 296 |
| Ab38 | GFTFADYA | 276 | ISGSGAAT | 282 | VSSGHWDFD | 289 | QSISSY | 279 | AASGLES | 28 | QQTYTVPLT | 296 |
| Ab39 | GFTFADYA | 276 | ISGSGAAT | 282 | VSSGHWDFD | 289 | QSISSY | 279 | AASVNES | 293 | QQTYTVPLT | 296 |
| Ab40 | GFTFADYA | 276 | ISGSGAAT | 282 | VSSGHWDFD | 289 | QSISSY | 279 | AASNEET | 294 | QQTYTVPLT | 296 |
| Ab41 | GFTFADYA | 276 | ISGSGAAT | 282 | VSSGHWDFD | 289 | QSISSY | 279 | AASNLES | 292 | QQTYGVPLT | 285 |
| Ab43 | GFTFADYA | 276 | ISGSGAAT | 282 | VSSGHWDYD | 287 | QSISSY | 279 | AASNLES | 292 | QQTYTVPLT | 296 |
| Ab44 | GFTFADYA | 276 | ISGSGAAT | 282 | VSSGHWDYD | 287 | QSISSY | 279 | AASGLES | 284 | QQTYTVPLT | 296 |
| Ab45 | GFTFADYA | 276 | ISGSGAAT | 282 | VSSGHWDYD | 287 | QSISSY | 279 | AASNLES | 292 | QQTYGVPLT | 285 |
| Ab46 | GFTFADYA | 276 | ISGSGAAT | 282 | VSSGHWDYD | 287 | QSISSY | 279 | AASGLES | 284 | QQTYGVPLT | 285 |
| Ab47 | GFTFADYA | 276 | ISGSGAAT | 282 | VSSGDWDYD | 290 | QSISSY | 279 | AASNLES | 292 | QQTYGVPLT | 285 |
| Ab48 | GFTFADYA | 276 | ISGSGAAT | 282 | VSSGDWDYD | 290 | QSISSY | 279 | AASGLES | 284 | QQTYGVPLT | 285 |
| Ab49 | GFTFADYA | 276 | ISGSGAAT | 282 | VSSGQWDYD | 291 | QSISSY | 279 | AASNLES | 292 | QQTYGVPLT | 285 |
| Ab50 | GFTFADYA | 276 | ISGSGAAT | 282 | VSSGQWDYD | 291 | QSISSY | 279 | AASGLES | 284 | QQTYGVPLT | 285 |
| Ab51 | GFTFADYA | 276 | ISGSGKAT | 288 | VSSGDWDYD | 290 | QSISSY | 279 | AASNLQS | 295 | QQTYTVPLT | 296 |
| Ab52 | GFTFADYA | 276 | ISGSGKAT | 288 | VSSGDWDYD | 290 | QSISSY | 279 | AASGLES | 284 | QQTYGVPLT | 285 |

In some embodiments, the antibody or an antigen-binding fragment thereof comprises a heavy chain variable domain (V_{H}) and a light chain variable domain (V_{L}), wherein, the V_{H} comprises an amino acid sequence having at least 90% (e.g., 90%, 91%, 92%, 93%, 94%, 95%, 96%, 97%, 98%, 99% and 100%) sequence identity to: EVQLLESGGGLVQPGGSLRLSCAASGFTFADYAMTWVRQAPGKGLEWVSAISGSGAATYFADSVKGRFTISRD NSKNTLYLQMNSLRAEDTAVYYCARVSSGHWDYDYWGQGTLVTVSS (SEQ ID NO: 297) and wherein the V_{L} comprises an amino acid sequence having at least 90% (e.g., 90%, 91%, 92%, 93%, 94%, 95%, 96%, 97%, 98%, 99% and 100%) sequence identity to: DIQLTQSPSSLSASVGDRVTITCRASQSISSYLNWYQQKPGKAPKLLIYAASGLESGVPSRFSGSGSGTDFTLTIS SLQPEDFATYYCQQTYGVPLTFGGGTKVEIK (SEQ ID NO: 298). In some embodiments, the antibody or the fragment binds an epitope that comprises one or more of the following amino acid residues of the proTGFβ1 polypeptide sequence: S35, G37, E38, V39, P40, P41, G42, P43, R274, K280, H283 and K309. Ab46 comprises the VH amino acid sequence of SEQ ID NO: 297 and the VL amino acid sequence of SEQ ID NO: 298.

In some embodiments, the antibody or the antigen-binding fragment comprises a heavy chain variable domain (V_{H}) and a light chain variable domain (V_{L}), wherein, the V_{H} comprises EVQLLESGGGLVQPGGSLRLSCAASGFTFADYAMTWVRQAPGKGLEWVSAISGSGAATYFADSVKGRFTISRD NSKNTLYLQMNSLRAEDTAVYYCARVSSGHWDYDYWGQGTLVTVSS (SEQ ID NO: 297) and wherein the V_{L} comprises

(SEQ ID NO: 298)
DIQLTQSPSSLSASVGDRVTITCRASQSISSYLNWYQQKPGK
APKLLIYAASGLESGVPSRFSGSGSGTDFTLTISSLQPEDFA
TYYCQQTYGVPLTFGGGTKVEIK.

In some embodiments, the antibody or the antigen-binding fragment comprises a heavy chain variable domain (V_{H}) and a light chain variable domain (V_{L}), wherein, the V_{H} comprises EVQLLESGGGLVQPGGSLRLSCAASGFTFADYAMTWVRQAPGKGLEWVSAISGSGAATYFADSVKGRFTISRD NSKNTLYLQMNSLRAEDTAVYYCARVSSGHWDFDYWGQGTLVTVSS (SEQ ID NO: 299) and wherein the V_{L} comprises

(SEQ ID NO: 300)
DIQLTQSPSSLSASVGDRVTITCRASQSISSYLNWYQQKPGKA
PKLLIYAASNLQSGVPSRFSGSGSGTDFTLTISSLQPEDFATY
YCQQTYTVPLTFGGGTKVEIK.

The disclosure includes nucleic acid sequences that encode any one of the amino acid sequences provided above. Encompassed herein are vectors (e.g., DNA plasmids, such as mammalian expression vectors, and related nucleic acid preparations) comprising the nucleic acid sequence; cells transfected with the vector(s); a cell line with stable expression of the nucleic acids; a cell culture comprising the cell, wherein optionally the cell culture comprises mammalian cells capable of large-scale production of the antibody or a protein construct comprising an antigen-binding fragment of the antibody.

In some embodiments, the monoclonal antibody or an antigen-binding fragment thereof, which selectively inhibits TGFβ1 activation, comprises a heavy chain complementary determining region 1 (CDRH1) having an amino acid sequence at least 95% identical to the sequence set forth in GFTFADYA (SEQ ID NO: 276); a heavy chain complementary determining region 2 (CDRH2) having an amino acid sequence at least 95% identical to the sequence set forth in ISGSGAAT (SEQ ID NO: 282); a heavy chain complementary determining region 3 (CDRH3) having an amino acid sequence at least 95% identical to the sequence set forth in VSSGHWDYD (SEQ ID NO: 287); a light chain complementary determining region 1 (CDRL1) having an amino acid sequence at least 95% identical to the sequence set forth in QSISSY (SEQ ID NO: 279); a light chain complementary determining region 2 (CDRL2) having an amino acid sequence at least 95% identical to the sequence set forth in AASGLES (SEQ ID NO: 284); and, a light chain complementary determining region 3 (CDRL3) having an amino acid sequence at least 95% identical to the sequence set forth in QQTYGVPLT (SEQ ID NO: 285).

In some embodiments, the monoclonal antibody or an antigen-binding fragment thereof, which selectively inhibits TGFβ1 activation, comprises a heavy chain complementary determining region 1 (CDRH1) having an amino acid sequence at least 96% identical to the sequence set forth in GFTFADYA (SEQ ID NO: 276); a heavy chain complementary determining region 2 (CDRH2) having an amino acid sequence at least 96% identical to the sequence set forth in ISGSGAAT (SEQ ID NO: 282); a heavy chain complementary determining region 3 (CDRH3) having an amino acid sequence at least 96% identical to the sequence set forth in VSSGHWDYD (SEQ ID NO: 287); a light chain complementary determining region 1 (CDRL1) having an amino acid sequence at least 96% identical to the sequence set forth in QSISSY (SEQ ID NO: 279); a light chain complementary determining region 2 (CDRL2) having an amino acid sequence at least 96% identical to the sequence set forth in AASGLES (SEQ ID NO: 284); and, a light chain complementary determining region 3 (CDRL3) having an amino acid sequence at least 96% identical to the sequence set forth in QQTYGVPLT (SEQ ID NO: 285).

In some embodiments, the monoclonal antibody or an antigen-binding fragment thereof, which selectively inhibits TGFβ1 activation, comprises a heavy chain complementary determining region 1 (CDRH1) having an amino acid sequence at least 98% identical to the sequence set forth in GFTFADYA (SEQ ID NO: 276); a heavy chain complementary determining region 2 (CDRH2) having an amino acid sequence at least 98% identical to the sequence set forth in ISGSGAAT (SEQ ID NO: 282); a heavy chain complementary determining region 3 (CDRH3) having an amino acid sequence at least 98% identical to the sequence set forth in VSSGHWDYD (SEQ ID NO: 287); a light chain complementary determining region 1 (CDRL1) having an amino acid sequence at least 98% identical to the sequence set forth in QSISSY (SEQ ID NO: 279); a light chain complementary determining region 2 (CDRL2) having an amino acid sequence at least 98% identical to the sequence set forth in AASGLES (SEQ ID NO: 284); and, a light chain complementary determining region 3 (CDRL3) having an amino acid sequence at least 98% identical to the sequence set forth in QQTYGVPLT (SEQ ID NO: 285).

In some embodiments, the monoclonal antibody or an antigen-binding fragment thereof, which selectively inhibits TGFβ1 activation, comprises a heavy chain complementary determining region 1 (CDRH1) having an amino acid sequence at least 99% identical to the sequence set forth in GFTFADYA (SEQ ID NO: 276); a heavy chain complementary determining region 2 (CDRH2) having an amino acid sequence at least 99% identical to the sequence set forth in ISGSGAAT (SEQ ID NO: 282); a heavy chain complementary determining region 3 (CDRH3) having an amino acid sequence at least 99% identical to the sequence set forth in VSSGHWDYD (SEQ ID NO: 287); a light chain complementary determining region 1 (CDRL1) having an amino acid sequence at least 99% identical to the sequence set forth in QSISSY (SEQ ID NO: 279); a light chain complementary determining region 2 (CDRL2) having an amino acid sequence at least 99% identical to the sequence set forth in AASGLES (SEQ ID NO: 284); and, a light chain complementary determining region 3 (CDRL3) having an amino acid sequence at least 99% identical to the sequence set forth in QQTYGVPLT (SEQ ID NO: 285).

In some embodiments, the monoclonal antibody or an antigen-binding fragment thereof, which selectively inhibits TGFβ1 activation, comprises a heavy chain complementary determining region 1 (CDRH1) having an amino acid sequence set forth in GFTFADYA (SEQ ID NO: 276); a heavy chain complementary determining region 2 (CDRH2) having an amino acid sequence set forth in ISGSGAAT (SEQ ID NO: 282); a heavy chain complementary determining region 3 (CDRH3) having an amino acid sequence set forth in VSSGHWDYD (SEQ ID NO: 287); a light chain complementary determining region 1 (CDRL1) having an amino acid sequence set forth in QSISSY (SEQ ID NO: 279); a light chain complementary determining region 2 (CDRL2) having an amino acid sequence set forth in AASGLES (SEQ ID NO: 284); and, a light chain complementary determining region 3 (CDRL3) having an amino acid sequence set forth in QQTYGVPLT (SEQ ID NO: 285). In one embodiment, the antibody or antigen-binding fragment thereof, comprises a heavy chain variable domain (V_{H}) comprising a sequence having at least 95% identity, 96% identity, 97% identity, 98% identity, 99% identity to, comprises, or consists of SEQ ID NO:297; and a light chain variable domain (V_{L}) comprising a sequence having at least 95% identity, 96% identity, 97% identity, 98% identity, 99% identity to, comprises, or consists of SEQ ID NO:298.

In some embodiments, the antibody or an antigen-binding fragment thereof comprises a heavy chain variable domain (VH) and a light chain variable domain (VL), wherein the VH has at least 90% (e.g., 90%, 91%, 92%, 93%, 94%, 95%, 96%, 97%, 98%, 99% and 100%) sequence identity to EVQLVQSGGVVVQPGGSLRLSCAASGFTFDDYTMHWVRQAPGKGLEWVSLISWDGGSTYYADSVKGRFTISRD NSKNSLYLQMNSLRTEDTALYYCAKDADDSTFDIWGQGTMVTVSS (SEQ ID NO: 1173) and wherein the VL has at least 90% (e.g., 90%, 91%, 92%, 93%, 94%, 95%, 96%, 97%, 98%, 99% and 100%) sequence identity to ETTLTQSPATLSVSPGERVTLSCRASQSVSRNLAWYQQKPGQPPKLLIYWASTRESGVPDRFSGSGSGTDFTLTI SSLQAEDVAVYYCQQYYSVPYTFGQGTKLEIK (SEQ ID NO: 1174). In some embodiments, the antibody or an antigen-binding fragment thereof comprises a heavy chain variable domain (VH) and a light chain variable domain (VL), wherein the VH has at least 90% (e.g., 90%, 91%, 92%, 93%, 94%, 95%, 96%, 97%, 98%, 99% and 100%) sequence identity to QMQLVQSGAEVKKPGSSVKVSCKASGGTFSSYAISWVRQAPGQGLEWMGGIIPMFGTTNYAQKFQGRVTIIADE STSTAYMELRSLRSDDTAVYYCARDREWEPAYGMDVWGQGTTVTVSS (SEQ ID NO: 1175) and wherein the VL has at least 90% (e.g., 90%, 91%, 92%, 93%, 94%, 95%, 96%, 97%, 98%, 99% and 100%) sequence identity to QSALTQPASVSGSPGQSITISCIGTSSDVGGYNYVSWYQQHPGKAPKLMIYDVSNRPSGVSNRFSGSKSGNTAS LTISGLQAEDEAMYYCSAYTVSSTWVFGGGTKVTVL (SEQ ID NO: 1176).

### Binding Kinetics of Antibodies

The antibodies and antigen-binding fragments thereof (e.g., Fabs) disclosed herein are characterized by enhanced binding properties. The antibodies and the antigen-binding fragments are capable of specifically binding to each of the presenting molecule-proTGFβ1 complexes (sometimes referred to as “Large Latency Complex” or LLC, which is a ternary complex comprised of a proTGFβ1 dimer coupled to a single presenting molecule), namely, LTBP1-proTGFβ1, LTBP3-proTGFβ1, GARP-proTGFβ1 and LRRC33-proTGFβ1. Recombinantly produced, purified protein complexes may be used as antigens (e.g., antigen complexes) to screen, evaluate or confirm the ability of an antibody to bind the antigen complexes in suitable in vitro binding assays. Such assays are well known in the art and include but are not limited to: Bio-Layer Interferometry (BLI)-based assays (such as OCTET®) and surface plasmon resonance (SPR)-based assays (such as BIACORE®).

Previously, antibodies and fragments that exhibited high affinities (e.g., sub-nanomolar K_{D}) to the LLCs were identified. Here, advantageously, antibodies and fragments with particularly slow dissociation rates were specifically selected, aimed to achieve particularly durable inhibitory effects.

### Methods of Treatment and Biomarkers of Therapeutic Efficacy

### Circulating/Circulatory MDSCs as a Biomarker

MDSCs are a heterogeneous population of cells named for their myeloid origin and their main immune suppressive function (Gabrilovich. Cancer Immunol Res. 2017 January; 5(1): 3-8). MDSCs generally exhibit high plasticity and strong capacity to reduce cytotoxic functions of T cells and natural killer (NK) cells, including their ability to promote T regulatory cell (Treg) expansion and in turn suppress T effector cell function (Gabrilovich et al., Nat Rev Immunol. (2012) 12:253-68). MDSCs are typically classified into two subsets, monocytic (m-MDSCs) and granulocytic (G-MDSCs or PMN-MDSCs), based on their expression of surface markers (Consonni et al., Front Immunol. 2019 May 3; 10:949). Suppressive G-MDSCs can be characterized by their production of reactive oxygen species (ROS) as the major mechanism of immune suppression. In contrast, M-MDSCs mediate immune suppression primarily by upregulating the inducible nitric oxide synthase gene (iNOS) and produce nitric oxide (NO) as well as an array of immune suppressive cytokines (Youn and Garilovich, Eur J Immunol. 2010 November; 40(11): 2969-2975).

MDSCs have been implicated in various diseases, such as chronic inflammation, infection, autoimmune diseases, and graft-versus-host diseases. In recent years, MDSCs have become an immune population of interest in cancer due to their role in inducing T cell tolerance through checkpoint blockade molecules such as the programmed death-ligand 1 (PD-L1) and the cytotoxic T-lymphocyte antigen 4 (CTLA4) (Trovato et al., J Immunother Cancer. 2019 Sep. 18; 7(1):255). Furthermore, MDSCs have generally been characterized as favoring tumor progression by mechanisms in addition to immune suppression, including promoting tumor angiogenesis. Studies to date have focused on MDSCs present in tumor biopsies, given their propensity to enrich around inflamed tissue. (Passro et al., Clin Transl Oncol. 2019 Jun. 28.; Ai et al., BMC Cancer. 2018 Dec. 5; 18(1):1220; Nakamura. Front Med (Lausanne). 2019; 6: 119). However, such studies had not been reported in the literature to have elucidated a clear relationship between MDSC levels and therapeutic response. For instance, low baseline monocytic MDSC frequency was shown to correlate poorly with treatment benefits (Pico de Coaña et al., Oncotarget. 2017 Mar. 28; 8(13): 21539-21553).

Many human cancers (e.g., solid tumors) are known to show elevated levels of MDSCs in biopsies from patients, as compared to healthy controls (reviewed, for example, in Elliott et al., (2017) Frontiers in Immunology, Vol. 8, Article 86). These human cancers include but are not limited to bladder cancer, colorectal cancer, prostate cancer, breast cancer, glioblastoma, hepatocellular carcinoma, head and neck squamous cell carcinoma, lung cancer, melanoma, NSCLC, ovarian cancer, pancreatic cancer, and renal cell carcinoma. The compositions and methods according to the present disclosure may be applied to one or more of these cancers.

Previously, it was demonstrated by Applicant that immunosuppressive tumors contain elevated levels of tumor-infiltrating or intratumoral MDSCs, also referred to as tumor-associated MDSCs, and evidence indicated that this was inversely correlated with anti-tumor immunity in a TGFβ1-dependent manner. For example, in MBT2 tumors, mice treated with a combination of Ab6 (TGFβ1-selective inhibitor) and a PD-1 antibody triggered a robust influx of cytotoxic CD8+ T cells and a corresponding reduction in the tumor-associated MDSC population (e.g., from about 11% to 1.4% of CD45+ cells). These data suggested that probing tumor-associated immune cells (e.g., MDSCs and/or CD8+ T cells), by, for example, biopsies, can be useful for characterizing anti-tumor effects in cancer patients. Further, Applicant made a surprising finding that relatively simple and noninvasive blood tests may provide equivalent information, leading to the recognition that pharmacological effects of TGFβ1 inhibition on overcoming an immunosuppressive phenotype can be determined by measuring circulating MDSC levels (e.g., circulating gMDSC levels).

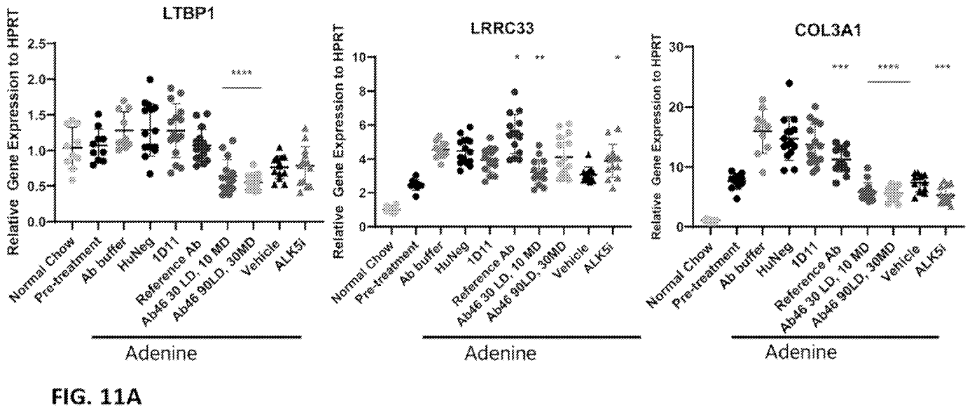

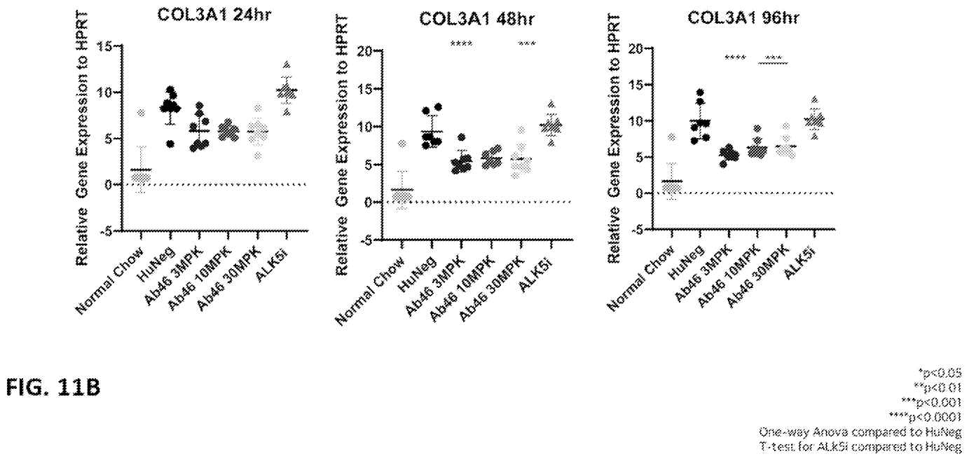

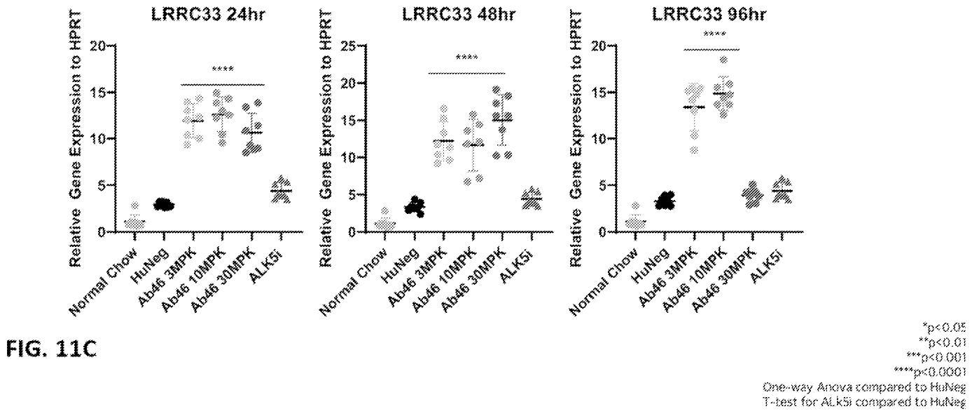

The present disclosure includes the finding that circulating MDSC levels (including gMDSCs and/or mMDSCs) may be determined by detecting or measuring LRRC33-positive cells in a blood sample, identifying LRRC33 as a novel blood-based biomarker for circulating MDSCs. See, FIGS. 11A and 11B. For example, LRRC33-positive cells in a blood sample collected from a patient (such as cancer patient) may be detected or measured by a FACS-based assay using an antibody that binds cell-surface LRRC33. In some embodiments, the LRRC33-expressing cells in a blood sample collected from a subject having cancer are G-MDSCs. While MDSCs are derived from bone marrow-originated monocytes, cell-surface expression of LRRC33 appears to be narrowly restricted to MDSCs, and not monocytes, in circulation. This recognition raises a new possibility of using LRRC33 as a blood-based marker for circulating MDSCs. LRRC33 expression may be determined using LRRC33-specific, or TGFβ1-LRRC33 complex-specific, antibodies. In some embodiments, LRRC33 expression may be determined by any of the antibodies disclosed in WO/2018/208888 and WO/2018/081287, the contents of which are incorporated herein in their entirety. Applicant has now established a correlation between circulatory MDSC levels and tumor-associated MDSC levels. Together with the finding that circulatory MDSCs appear to show robust and uniform cell-surface LRRC33 expression, determination of LRRC33 levels measured in blood samples may serve as an effective surrogate to assess tumor immune-phenotype, such as immunosuppression, without the need for more invasive procedures such as tumor biopsy.

In various embodiments, the present disclosure provides methods of treating cancer, predicting, or determining efficacy, and/or confirming pharmacological response by monitoring the levels of circulating MDSCs (e.g., circulating gMDSCs) in a sample obtained from a patient (e.g., in the blood or a blood component of a patient) receiving a TGFβ inhibitor, e.g., a TGFβ1-selective inhibitor (such as a selective pro- or latent-TGFβ1 inhibitor, e.g., Ab6), isoform-non-selective TGFβ inhibitors (such as low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors), and/or an integrin inhibitor (and integrin inhibitors (e.g., antibodies that bind to αVβ1, αVβ3, αVβ5, αVβ6, αVβ8, α5β1, αIIbβ3, or α8β1 integrins, and inhibit downstream activation of TGFβ. e.g., selective inhibition of TGFβ1 and/or TGFβ3). Exemplary integrin inhibitors include the anti-αVβ8 integrin antibodies provided in WO2020051333, the disclosure of which is incorporated by reference. In various embodiments disclosed herein, the circulating MDSCs may be measured within 1, 2, 3, 4, 5, 6, or 7 days, or within 1, 2, 3, 4, 5, 6, 7, 8, 9, or 10 weeks (e.g., preferably less than 6 weeks) following administration of a treatment to a subject, e.g., administration of a therapeutic dose of a TGFβ inhibitor.

In certain embodiments, the TGFβ treatment may be administered alone or in conjunction with an additional cancer therapy. The treatment may be administered to subjects with an immunosuppressive cancer or a myeloproliferative disorder. In some embodiments, the TGFβ inhibitor is a TGFβ1-selective antibody or antigen-binding fragment thereof encompassed in the current disclosure (e.g., Ab6). In some embodiments, the TGFβ1 -selective antibody or antigen-binding fragment does not inhibit TGFβ2 and TGFβ3 at a therapeutically effective dose. In some embodiments, the TGFβ inhibitor is an isoform-non-selective TGFβ inhibitor (such as low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors). In some embodiments, the TGFβ inhibitor is an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). Exemplary integrin inhibitors include the anti-αVβ8 integrin antibodies provided in WO2020051333, the disclosure of which is incorporated by reference. In some embodiments, the additional cancer therapy may include chemotherapy, radiation therapy (including radiotherapeutic agents), cancer vaccine or immunotherapy including checkpoint inhibitor therapies such as anti-PD-1, anti-PD-L1, and anti-CTLA-4 antibodies. In some embodiments, the checkpoint inhibitor therapy is selected from the group consisting of ipilimumab (e.g., Yervoy®); nivolumab (e.g., Opdivo®); budigalimab (ABBV-181); pembrolizumab (e.g., Keytruda®); avelumab (e.g., Bavencio®); cemiplimab (e.g., Libtayo®); atezolizumab (e.g., Tecentriq®); and durvalumab (e.g., Imfinzi®). In preferred embodiments, a combination cancer therapy comprises Ab6 and at least one checkpoint inhibitor (such as those listed above). Thus, in some embodiments, a combination of Ab6 and a checkpoint inhibitor is used for the treatment of cancer in a human patient in amounts effective to treat the cancer. In some embodiments, the TGFβ treatment may further or alternatively include a second checkpoint inhibitor and/or chemotherapy.

For example, without being bound by theory, evidence suggests that overactive TGFβ pathways may correlate with unresponsiveness of a tumor to genotoxic therapies, such as chemotherapy and radiation therapy (Liu et al., Sci Transl Med. 2021 Feb. 10; 13(580):eabc4465). This is observed across multiple cancer types, e.g., cancers of the epithelia, e.g., carcinoma. In certain embodiments, such cancer types include ovarian cancer, breast cancer, bladder cancer, pancreatic cancer, e.g., pancreatic adenocarcinoma, prostate cancer, e.g., prostate adenocarcinoma, melanoma, e.g., skin cutaneous melanoma, lung cancer, e.g., lung squamous cell carcinoma and lung adenocarcinoma, liver cancer (e.g., liver hepatocellular carcinoma), uterine cancer, e.g., uterine corpus endometrial carcinoma, kidney cancer, e.g., renal clear cell carcinoma, head and neck cancer, e.g., head and neck squamous cell carcinoma, colon cancer, e.g., colon adenocarcinoma, esophageal carcinoma, and tenosynovial giant cell tumor (TGCT). Accordingly, TGFβ inhibitors (e.g., Ab6) may be used in conjunction with one or more genotoxic therapies (e.g., chemotherapy and/or radiation therapy, including radiotherapeutic agents) to treat such a cancer in a subject. In certain embodiments, such a cancer may have elevated TGFβ levels, e.g., elevated TGFβ activity, as indicated by direct measurement and/or one or more changes in downstream gene regulation (e.g., in one or more genes involved in DNA repair). For instance, a cancer, such as one of the cancers listed above, may have elevated TGFβ signaling as indicated by upregulation of one or more genes associated with non-homologous end joining (NHEJ), e.g., Cyclin Dependent Kinase Inhibitor 1A (CDKN1A), or downregulation of one or more genes relating to alternative end joining, e.g., LIG1 (DNA ligase 1), PARP1, and/or POLQ. In some embodiments, the cancer is a cancer having elevated TGFβ1 levels associated with ROS (e.g., elevated ROS). Without being bound by theory, ROS may induce an increase in TGFβ levels (e.g., TGFβ1 levels) which may be reduced by a TGFβ inhibitor (e.g., a TGFβ1 inhibitor) disclosed herein.

The present disclosure also provides methods of using measurements of circulating MDSCs in treating cancer in subjects administered a TGFβ inhibitor alone or in conjunction with an immunotherapy. Furthermore, the descriptions presented herein provide support for the circulating MDSC population (e.g., the circulating gMDSC population) as an early predictive marker of efficacy, particularly in cancer subjects treated with a TGFβ inhibitor and checkpoint inhibitor combination therapy, e.g., at a time point before other markers of treatment efficacy, such as a reduction in tumor volume, can be detected.

In certain embodiments, a TGFβ inhibitor, e.g., a TGFβ1-selective inhibitor such as Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3) is administered concurrently (e.g., simultaneously), separately, or sequentially to a checkpoint inhibitor therapy such that the amount (e.g., dose) of TGFβ1 inhibition administered is sufficient to reduce circulating MDSC levels by at least 10%, at least 15%, at least 20%, at least 25%, or more, as compared to baseline MDSC levels. In some preferred embodiments, circulating MDSC levels are circulating gMDSC levels. Circulating MDSC levels may be measured prior to or after each treatment or each dose of the TGFβ inhibitor such that a decrease of at least 10%, at least 15%, at least 20%, at least 25%, or more in circulating MDSC levels may be indicative or predictive of treatment efficacy. In some embodiments, the level of circulating MDSCs may be used to determine disease burden (e.g., as measured by a change in relative tumor volume before and after a treatment regimen). In certain embodiments, a decrease in circulating MDSC levels may be indicative of a decrease in disease burden (e.g., a decrease in relative tumor volume). For instance, circulating MDSC levels may be measured prior to and after the administration of a dose of TGF inhibitor (such as isoform-selective inhibitors, e.g., Ab6, isoform-non-selective TGFβ inhibitors, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ, e.g., selective inhibition of TGFβ1 and/or TGFβ3) and a reduction in circulating MDSC levels may be indicative or predictive of pharmacological effects, e.g., of a reduction in disease burden (e.g., a reduction in relative tumor size). In certain embodiments, circulating MDSC levels may be measured prior to and following administration of a first dose of a TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3). In some embodiments, administration of a first dose of TGFβ inhibitor (e.g., Ab6, isoform-non-selective TGFβ inhibitors, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3) may be used to reduce tumor volume, such that administration of the TGFβ inhibitor reduces circulating MDSC levels by at least 10%, at least 20%, at least 25%, or more, as compared to circulating MDSC levels prior to administration. In some embodiments, reduction in circulating MDSC levels is indicative or predictive of pharmacological effects and further warrants administration of a second or more dose(s) of the TGFβ inhibitor. In some embodiments, the first dose of the TGFβ inhibitor is the very first dose of TGFβ inhibitor received by the patient. In some embodiments, the first dose of the TGFβ inhibitor is the first dose of a given treatment regimen comprising more than one dose of TGFβ inhibitor. In another embodiment, circulating MDSC levels may be measured prior to and after combination treatment comprising a TGFβ inhibitor (e.g., Ab6) and a checkpoint inhibitor therapy, administered concurrently (e.g., simultaneously), separately, or sequentially, and a reduction in circulating MDSC levels is indicative or predictive of therapeutic efficacy. In some embodiments, the reduction of circulating MDSC levels following the combination treatment of a TGFβ inhibitor, such as a TGFβ1 inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3), and a checkpoint inhibitor therapy, may warrant continuation of treatment.

In certain embodiments of the present disclosure, levels of circulating MDSCs (e.g., circulating gMDSCs) may be used to predict, determine, and monitor pharmacological effects of treatment comprising a dose of TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) administered alone or in conjunction with another cancer therapy such as a checkpoint inhibitor. In certain embodiments, circulating MDSCs may be measured within six weeks following administration of the initial treatment (e.g., the (first) dose of TGFβ inhibitor). In certain embodiments, circulating MDSC levels may be measured within thirty days following administration of the initial dose of TGFβ inhibitor. In some embodiments, MDSC levels may be measured within or at about three weeks following administration of the initial dose of TGFβ inhibitor. In some embodiments, MDSC levels may be measured within or at about two weeks following administration of the initial dose of TGFβ inhibitor. In some embodiments, MDSC levels may be measured within or at about ten days following administration of the initial dose of TGFβ inhibitor.

In certain embodiments, circulating MDSC levels (e.g., circulating gMDSC levels) may be used to select, inform treatment in, and/or predicting response in patients who have not received a checkpoint inhibitor treatment previously. Patients diagnosed with a cancer type with reported high response rates to checkpoint inhibitor therapy (e.g., overall response rate of greater than 30%, greater 40%, greater than 50%, or greater, as reported in the art) who have not received a checkpoint inhibitor therapy previously may be tested to first determine whether their tumors exhibit an immune-excluded, immune-infiltrated, or immunosuppressive phenotype. In some embodiments, circulating MDSCs may be used in conjunction with immunohistochemistry, flow cytometry, and/or in vivo imaging methods known in the art to determine the immune phenotype of the tumor. Patients with cancers exhibiting an immune-excluded and/or immunosuppressive phenotype may be selected to receive a TGFβ inhibitor. Patients with cancers exhibiting an immune-infiltrated phenotype may also be selected to receive a TGFβ1 inhibitor if the patients' circulating MDSC levels (e.g., circulating gMDSC levels) indicate that the cancer exhibits an immunosuppressive phenotype (e.g., the circulating MDSC level or gMDSC level is higher than a threshold level, and is, for instance, higher than the circulating MDSC or gMDSC level in a healthy control subject or higher than the circulating MDSC or gMDSC level in a control patient with a cancer that is not immunosuppressive). In some embodiments, the circulating MDSC or gMDSC level is higher than a threshold level if the circulating MDSC or gMDSC level is detectable. In some embodiments, the threshold circulating MDSC or gMDSC level is above 1% of the white blood cell component/PBMC component in a blood sample, such as above 1%, 2%, 3%, 4%, 5%, 6%, 7%, 8%, 9%, 10%, 15%, 20%, 25%, 30%, 35%, 40%, 45%, 50%, 55%, or 60%. The TGFβ inhibitor may be a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) and checkpoint inhibitor combination therapy (e.g., an anti-PD1 or anti-PD-L1 antibody). Circulating MDSC levels (e.g., circulating gMDSC levels) may be further monitored as an early predictor of treatment response. In certain embodiments, patients diagnosed with a cancer type with reported low response rates to checkpoint inhibitor therapy (e.g., overall response rate of 30% or less, 20% or less, or 10%, or less, as reported in the art) who have not received a checkpoint inhibitor therapy previously may be treated with a combination of a TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) and a checkpoint inhibitor therapy. In some embodiments, treatment response in these patients may be predicted by monitoring circulating MDSC levels (e.g., circulating gMDSC levels).

In certain embodiments, circulating MDSC levels (e.g., circulating gMDSC levels) may be used for selecting, informing treatment in, and predicting response in patients who are resistant to checkpoint inhibitor therapy or who do not tolerate checkpoint inhibitor therapy (e.g., due to adverse effects). These patients may have primary resistance (i.e., have never shown response to checkpoint inhibitor therapy) or have acquired resistance (i.e., have responded checkpoint inhibitor therapy initially and developed resistance over time). In some embodiments, resistance to checkpoint inhibitor therapy in patients is indicative of immune suppression and/or exclusion, thus these patients may be selected as candidates for receiving a TGFβ inhibitor therapy, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3). In certain embodiments, patients with either primary resistance or acquired resistance to checkpoint inhibitor may be administered a TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3), and their response to treatment may be monitored and/or predicted by circulating MDSC levels. In some embodiments, a reduction of at least 10%, at least 15%, at least 20%, at least 25%, or more in circulating MDSC levels may be indicative of response to the TGFβ inhibitor therapy. In some embodiments, a reduction of at least 10%, at least 15%, at least 20%, at least 25%, or more in circulating MDSC levels may indicate pharmacological effects of a treatment, e.g., with a TGFβ inhibitor. In certain embodiments, a decrease in circulating MDSC levels may be indicative of a decrease in tumor size.

Most TGFβ inhibitors currently in development are not isoform-selective. These include pan-inhibitors of TGFβ, and inhibitors that target TGFβ1/2 and TGFβ1/3. Approaches taken to manage possible toxicities associated with such inhibitors include careful dosing regimens to hit a narrow window in which both efficacy and acceptable safety profiles may be achieved. This may include sparing of an isoform non-selective inhibitor, which may include infrequent dosing and/or reducing dosage per administration. For instance, in lieu of weekly dosing of a biologic TGFβ inhibitor, monthly dosing may be considered. Another example is to dose only in an initial phase of a combination immunotherapy so as to avoid or minimize toxicities associated with TGFβ inhibition.

Because a combination therapy comprising a cancer therapy (such as checkpoint inhibitor therapy) and an isoform-non-selective TGFβ inhibitor may result in a greater risk of toxicity as compared to a TGFβ1-selective inhibitor (e.g., Ab6), in order to mitigate or manage such risk, the isoform-non-selective TGFβ inhibitor may be administered infrequently or intermittently, for example on an “as-needed” basis. In such treatment paradigm, circulating MDSC levels (e.g., circulating gMDSC levels) may be monitored periodically in order to determine that the effects of overcoming immunosuppression are sufficiently maintained, so as to ensure antitumor effects of the cancer therapy. During the course of cancer treatment, if MDSCs become elevated, it indicates that the patient benefits from additional doses of a TGFβ inhibitor. Such approach may help reduce unnecessary risk and adverse events associated with TGFβ inhibition, non-isoform-selective inhibitors in particular. In some embodiments, the TGFβ inhibitor targets TGFβ1/2. In some embodiments, the TGFβ inhibitor targets TGFβ1/3. In some embodiments, the TGFβ inhibitor targets TGFβ1/2/3. In some embodiments, the TGFβ inhibitor selectively targets TGFβ1.

Accordingly, the present disclosure provides a TGFβ inhibitor for use in an intermittent dosing regimen for cancer immunotherapy in a patient, wherein the intermittent dosing regimen comprises the following steps: measuring circulating MDSCs (e.g., circulating gMDSCs) in a first sample collected from the patient prior to a TGFβ inhibitor treatment; administering a TGFβ inhibitor to the patient treated with a cancer therapy, wherein the cancer therapy is optionally a checkpoint inhibitor therapy; measuring circulating MDSCs in a second sample collected from the patient after the TGFβ inhibitor treatment; continuing with the cancer therapy if the second sample shows reduced levels of circulating MDSCs as compared to the first sample; measuring circulating MDSCs in a third sample; and, administering to the patient an additional dose of a TGFβ inhibitor, if the third sample shows elevated levels of circulating MDSC levels as compared to the second sample. In some embodiments, the TGFβ inhibitor is an isoform-non-selective inhibitor. In some embodiments, the sample is blood or a blood component sample. In some embodiments, the isoform-non-selective inhibitor inhibits TGFβ1/2/3, TGFβ1/2 or TGFβ1/3. Baseline circulating MDSC levels are likely to be elevated in cancer patients as compared to healthy individuals, and subjects with immunosuppressive cancers may have even more elevated circulating MDSC levels. As such, decreases in circulating MDSC levels in patients treated with a TGFβ inhibitor therapy such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3), either alone or in combination with a checkpoint inhibitor therapy, may be indicative of a reduction or reversal of immune suppression in the cancer. In certain embodiments, a TGFβ inhibitor, such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3) is administered to a subject with cancer such that the dose of the TGFβ inhibitor is sufficient to reduce or reverse immune suppression in the cancer as indicated by a reduction of circulating MDSC levels and/or a change in the levels of tumor-associated immune cells measured after administering the TGFβ inhibitor treatment as compared to levels measured before administration. In some embodiments, levels of circulating MDSC and/or tumor-associated immune cells are measured before and after administration of a TGFβ inhibitor treatment such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3) in combination with a checkpoint inhibitor therapy, and a reduction of circulating MDSC levels and/or change(s) in the levels of tumor-associated immune cells measured after treatment as compared to levels measure before treatment indicates reduction or reversal of immune suppression in the cancer.

Circulating MDSC levels may be determined in a sample such as a whole blood sample or a blood component (e.g., PBMCs). In some embodiments, the sample is fresh whole blood or a blood component of a sample that has not been previously frozen. In certain embodiments, circulating MDSCs may be collected by drawing peripheral blood into heparinized tubes. From peripheral blood, peripheral blood mononuclear cells may be isolated using, e.g., elutriation, magnetic beads separation, or density gradient centrifugation methods (e.g., Ficoll-Paque®) known in the art. In some embodiments, MDSCs may be separated from peripheral blood mononuclear cells by CD11 b+ marker selection (e.g., using CD11 b+ microbeads or antibodies). G-MDSCs and M-MDSCs may be further distinguished from CD11 b+ cells via e.g., flow cytometry/FACS analysis based on surface marker expression. For example, human G-MDSCs may be identified by expression of the cell-surface markers CD11b, CD33, CD15 and CD66b. In some embodiments, human G-MDSCs may also express LOX-1, Arginase, and/or low levels of HLA-DR. Human M-MDSCs may be identified by expression of the cell surface markers CD11 b, CD33 and CD14, as well as low levels of HLA-DR in some embodiments. Quantification of circulating MDSCs may be represented as percentage of total CD45+ cells.

### Tumor-Associated Immune Cell Markers

Immune cell markers may be used to determine whether a cancer has an immune-excluded phenotype, and/or may be used in determining treatment efficacy or treatment regimen, alone or in combination with other circulating biomarkers such as circulating MDSCs. If the tumor is determined to have an immune-excluded phenotype, cancer therapy (such as CBT) alone may not be efficacious. Without being bound by theory, the tumor may lack sufficient cytotoxic cells within the tumor environment for effective CBT treatment alone. Thus, an alternative and/or add-on therapy with a TGFβ inhibitor (such as those described herein) may reduce immuno-suppression, thereby providing an improved treatment alone or rendering the resistant tumor more responsive to a cancer therapy. In some embodiments, immune cell markers are measured in biopsies (e.g., core needle biopsies). In some embodiments, patients having an immune-excluded tumor are administered a treatment comprising one or more TGFβ inhibitor (e.g., TGFβ1 inhibitor, e.g., Ab6). In some embodiments, patients having an immune-excluded tumor are administered a treatment comprising one or more TGFβ inhibitor (e.g., TGFβ1 inhibitor, e.g., Ab6) inhibitor and monitored for improvement in condition (e.g., increased immune cell penetration into a tumor, reduced tumor volume, etc.). In some embodiments, a patient exhibiting an improvement in condition after a first round of treatment is administered one or more additional rounds of treatment. In some embodiments, subjects are administered one or more additional treatment in combination with the one or more TGFβ inhibitor (e.g., TGFβ1 inhibitor, e.g., Ab6).

Tumor-associated immune cells that may be used to indicate the immune contexture of a tumor/cancer microenvironment include, but are not limited to, cytotoxic T cells and tumor-associated macrophages (TAMs), as well as tumor-associated MDSCs. Biomarkers to detect cytotoxic T cell levels may include, but are not limited to, the CD8 glycoprotein, granzyme B, perforin, and IFNγ, of which the latter three markers may also be indicative of activated cytotoxic T cells. To measure the level of TAMs, protein markers such as HLA-DR, CD68, CD163, CD206, and other biomarkers, any method known in the art may be used. In certain embodiments, increased levels of cytotoxic T cells, e.g., activated cytotoxic T cells, detected within the tumor microenvironment may be indicative of reduction or reversal of immune suppression. For example, an increase in CD8 expression and perforin, granzyme B, and/or IFNγ expression by tumor-associated immune cells may be indicative of reduction or reversal of immune suppression in the cancer. In certain embodiments, decreased levels of TAMs or tumor-associated MDSCs detected within the tumor microenvironment may be indicative of reduced or reversal of immune suppression. For example, a decrease of HLA-DR, CD68, CD163, and CD206 expression by tumor-associated immune cells may indicate reduced or reversal of immune suppression in the cancer. In certain embodiments, tumor-associated immune cells, e.g., CD8+ T cells, may be used in combination with one or more additional biomarkers to indicate immune contexture of a tumor/cancer microenvironment. In certain embodiments, the immune contexture of a tumor may be characterized by the density, location, organization, and/or functional orientation of tumor-infiltrating immune cells. In certain embodiments, such markers may be used to determine the immune phenotype of a tumor, e.g., to determine if a tumor is immune excluded, inflamed, or desert.

In various embodiments, cytotoxic T cells, e.g., in a patient sample, may be used to determine whether a cancer has an immune-excluded phenotype, and/or may be used in determining treatment efficacy or treatment regimen, alone or in combination with other biomarkers such as circulating MDSCs. For example, CD8 expression and/or the distribution of CD8 expression in a tumor sample may be used. For instance, CD8 expression may be examined in a sample to determine distribution in the tumor (i.e., tumor compartment), stroma (i.e., stroma compartment), and margin (i.e., margin compartment; identified, e.g., by assessing the region approximately 10-100 μm, or 25-75 μm, or 30-60 μm, e.g., 50 μm, between tumor and stroma). In certain embodiments, tumor, stroma, and/or margin compartments within the tumor may be identified using histological methods (e.g., pathologist assessment, pathologist-trained machine learning algorithms, and/or immunohistochemistry). In certain embodiments, CD8+ T cells in a tumor compartment may be referred to as “tumor-associated CD8+ cells”. In certain embodiments, CD8+ T cells in a stroma compartment may be referred to as “stroma-associated CD8+ cells”. In certain embodiments, CD8+ T cells in a margin compartment may be referred to as “margin-associated CD8+ cells”. In some embodiments, CD8 distribution may be determined in a tumor nest (e.g., a mass of cells extending from a common center seen in a cancerous growth; in an embodiment, tumor nests comprise at least 250 cells and at least 500 μm²), the stroma surrounding the tumor nest, and the margin between the tumor nest and its surrounding stroma (identified, e.g., by assessing the region approximately 10-100 μm, or 25-75 μm, or 30-60 μm, e.g., 50 μm, between the tumor nest and the surrounding stroma). In certain embodiments, tumor nests may be identified using histological methods (e.g., pathologist assessment, pathologist-trained machine learning algorithms, and/or immunohistochemistry). In certain embodiments, one or more tumor nests may be found within a tumor compartment. In certain embodiments, a tumor may comprise multiple (e.g., at least 5, at least 10, at least 20, at least 25, at least 50, or more) tumor nests. By default, unless otherwise indicated by context, the term “stroma” or “stroma compartment” refers to the stroma surrounding the tumor, and the term “margin” or “margin compartment” refers to the margin between the tumor and the stroma surround the tumor. In some embodiments, the structural interface between the tumor/tumor nest and the surrounding stroma is determined by imaging analysis. A margin can then be defined as the region surrounding the interface in either direction by a predetermined distance, for example, 10-100 μm. In some embodiments, this distribution may be used prior to administering a TGFβ inhibitor, such as a TGFβ1 inhibitor (e.g., Ab6) to select a patient for treatment and/or predict and/or determine the likelihood of a therapeutic response (e.g., an anti-tumor response) to an anti-cancer therapy comprising an anti-TGFβ inhibitor. For instance, if no or few cytotoxic T cells (e.g., less than 5% CD8+ T cells) are seen in a tumor sample, including in stroma and margin, this may indicate a patient who would not benefit from TGFβ inhibitor therapy (without being bound by theory, this may be because there are few immune cells to recruit to the tumor). Similarly, if a high density of cytotoxic T cells (e.g., greater than 5% CD8+ T cells) is observed in tumor as well as stroma and margin, this patient may also have limited benefit from TGFβ inhibitor therapy (without being bound by theory, this may be because immune cells have already infiltrated the tumor). In contrast, in certain embodiments, the subject's cancer may exhibit an immune-excluded phenotype, in which cytotoxic T cells (e.g., CD8+ T cells) are observed clustered primarily in or near the margin, e.g., at the border between the margin and the tumor, and not significantly infiltrated into the tumor itself (e.g., less than 5% CD8+ T cells in the tumor compartment and greater than 10% CD8+ T cells in the margin and/or stroma compartment). In certain embodiments, the subject's cancer may exhibit an immune-excluded phenotype, in which cytotoxic T cells (e.g., CD8+ T cells) are observed clustered primarily in or near the margin, e.g., at the border between the margin and the tumor (or peri-vasculature), and not significantly infiltrated into the tumor core itself (e.g., less than 5% CD8+ T cells in the tumor compartment and greater than 5% CD8+ T cells in the margin and/or stroma compartment). In certain embodiments, the subject's cancer may exhibit an immune-excluded phenotype, in which cytotoxic T cells (e.g., CD8+ T cells) are observed clustered primarily in or near the margin, e.g., at the border between the margin and the tumor, and not significantly infiltrated into the tumor itself (e.g., less than 5%, less than 10%, less than 15%, or fewer CD8+ T cells in the tumor compartment and greater than 5%, greater than 10%, greater than 15%, or more CD8+ T cells in the margin and/or stroma compartment). In some embodiments, CD8+ content in tumor compartments may be based on any of the methods described in Ziai et al. (PloS One. 2018; 13(1): e0190158), Massi et al. (J Immunother Cancer. 2019 Nov. 15; 7(1):308), Sharma et al. (Proc Natl Acad Sci USA. 2007 Mar. 6; 104(10):3967-72), or Echarti et al. (Cancers (Basel). 2019 September; 11(9): 1398), the contents of which are hereby incorporated in their entirety. Any of these methods may be used to determine the immune phenotype of the tumor. Tumor samples with this pattern from a patient may indicate a patient likely to benefit from TGFβ inhibitor therapy (without being bound by theory, this may be because the tumor is actively suppressing the immune response, preventing sufficient ingress of cytotoxic T cells, which could be partially or completely reversed by the TGFβ inhibitor).

In some embodiments, an immune-excluded phenotype is characterized by determining a cluster score of cytotoxic T cells (e.g., CD8+ T cells) within a tumor-associated compartment, e.g., in the tumor, in the margin near the external perimeters of a tumor mass, and/or in the vicinity of tumor vasculatures. In some embodiments, the cluster score of cytotoxic T cells (e.g., CD8+ T cells) can be determined based on the homogeneity of immune cells in a particular tumor-associated compartment, such that a compartment containing highly uniform distribution of cytotoxic T cells (e.g., CD8+ T cells) yields a high cluster score. In certain embodiments, tumors exhibiting an immune-excluded phenotype may be characterized by lower densities of cytotoxic T cells (e.g., CD8+ T cells) inside the tumor as compared to densities outside of the tumor (e.g., the external perimeters of a tumor mass and/or near the vicinity of vasculatures of a tumor). In some embodiments, the immune-excluded phenotype is characterized by cytotoxic T cells (e.g., CD8+ T cells) in the tumor stroma that are located in close vicinity (e.g., less than 100 μm) to the tumor. In some embodiments, the immune-excluded phenotype is characterized by cytotoxic T cells (e.g., CD8+ T cells) capable of infiltrating the tumor nest and locating at a close distance (e.g., less than 100 μm) to the tumor. In some embodiments, CD8+ T cells can be observed in clusters within a tumor near intratumoral blood vessels as determined for example by endothelial markers. By comparison, upon overcoming immunosuppression by TGF beta inhibitors, more uniform distribution of CD8+ T cells within the tumor can be observed, presumably as a result of the CD8+ cells being able to infiltrate from the perivascular regions and possibly proliferate in the tumor.

In certain embodiments, levels of tumor-infiltrating cytotoxic T cells (e.g., CD8+ T cells) and their activation status may be determined from a tumor biopsy sample obtained from the subject. In some embodiments, tumor biopsy samples, e.g., core needle biopsies, may be obtained at least 28 days prior to and at least 100 days following treatment administration. In some embodiments, tumor biopsy samples, e.g., core needle biopsies, may be obtained about 21 days to about 45 days following treatment administration. In some embodiments, tumor biopsy samples may be obtained via core needle biopsy. In some embodiments, treatment is continued if an increase is detected.

In certain embodiments, the immune phenotype of a subject's cancer may be determined by measuring the cell densities of cytotoxic T cells (e.g., percent of CD8+ T cells per square millimeter or other defined square distance) in a tumor biopsy sample. In certain embodiments, the immune phenotype of a subject's cancer may be determined by comparing the densities of cytotoxic T cells (e.g., CD8+ T cells) inside the tumor to that outside the tumor (e.g., to cells in the margin, e.g., at the external perimeters of a tumor mass and/or near the vicinity of vasculatures of a tumor). In some embodiments, the immune phenotype of a subject's cancer may be determined by comparing the percentage of CD8+ lymphocytes inside the tumor to that outside the tumor. In certain embodiments, the immune phenotype of a subject's cancer may be determined by comparing the cluster or dispersion of cytotoxic T cells (e.g., average number of CD8+ T cells surrounding other CD8+ T cells) in the tumor, stroma, or margin. In certain embodiments, the immune phenotype of a subject's cancer may be determined by measuring the average distance from cytotoxic T cells (e.g., CD8+ T cells) in the stroma to the tumor. In certain embodiments, the immune phenotype of a subject's cancer may be determined by measuring the average depth of cytotoxic T cell (e.g., CD8+ T cell) penetration into the tumor nest. Cell counts and density may be determined using immunostaining and computerized or manual measurement protocols. In certain embodiments, levels of cytotoxic T cells (e.g., CD8+ T cells) may be measured using immunohistochemical analysis of tumor biopsy samples. In certain embodiments, levels of cytotoxic T cells (e.g., CD8+ T cells) may be determined at least 28 days prior to and/or at least 100 days following administering a TGFβ therapy. In certain embodiments, levels of cytotoxic T cells (e.g., CD8+ T cells) may be determined up to about 45 days (e.g., about 21 days to about 45 days) following administering a TGFβ therapy. In some embodiments, levels of cytotoxic T cells (e.g., CD8+ T cells) are determined 5, 10, 15, 20, 25, 30, or more days prior to and/or at least 50, 60, 70, 80, 90, 100, 110, 120, 130, 140, or 150 days following administering a TGFβ therapy (or at any time point in between).

In some embodiments, a tumor with lower levels of cytotoxic T cells (e.g., CD8+ T cells) inside the tumor as compared to cytotoxic T cell levels (e.g., CD8+ T cells) outside the tumor (e.g., the external perimeters of a tumor and/or near the vicinity of vasculatures of a tumor) may be identified as an immune-excluded tumor. In some embodiments, immune-excluded tumors may also have higher levels of cytotoxic T cells (e.g., CD8+ T cells) in the tumor stroma as compared to inside the tumor. In certain embodiments, immune-excluded tumors may be identified by determining the ratio of cytotoxic T cell density (e.g., CD8+ T cells) inside the tumor to outside of the tumor, wherein the ratio is less than 1. In certain embodiments, immune-excluded tumors may be identified by determining the cytotoxic T cell density ratio inside the tumor to density in the tumor margin, wherein the ratio is less than 1. In certain embodiments, immune-excluded tumors may be identified by determining the cell density ratio inside the tumor to density in the tumor stroma, wherein the ratio is less than 1. In certain embodiments, immune-excluded tumors may be identified by comparing the absolute number, percentage, and/or density of cytotoxic T cells (e.g., CD8+ T cells) inside the tumor to outside the tumor (e.g., margin and/or stroma). In some embodiments, the absolute number, percentage, and/or density of cytotoxic T cells (e.g., CD8+ T cells) outside the tumor is at least 2-fold, 3-fold, 4-fold, 5-fold, 7-fold, or 10-fold greater than inside the tumor in an immune-excluded tumor. In some embodiments, an immune-excluded tumor comprises less than 5% CD8+ T cells inside the tumor and greater than 10% CD8+ T cells in the tumor margin and/or stroma. In some embodiments, immune-excluded tumors may be identified by comparing a ratio of compartmentalized cytotoxic T cell density (e.g., density of CD8+ cells inside the tumor to density in the tumor margin and/or stroma) and the ratio of whole tissue cytotoxic T cell density (e.g., CD8+ cells inside the tumor to CD8+ cells in the entire tumor tissue or biopsy), wherein the compartmentalized ratio is greater than the whole tissue ratio. In some embodiments, a tumor with increased cell density of cytotoxic T cells (e.g., CD8+ T cells) at an average distance of about 100 μm or less outside of the tumor may be identified as an immune-excluded tumor. In some embodiments, cytotoxic T cell density (e.g., CD8+ T cells) may be used in conjunction with one or more parameters, such as average CD8+ cluster score. In some embodiments, an average CD8+ clustering score of 50% or less in the tumor indicates immune exclusion.

In some embodiments, a tumor with lower levels of CD8+ T cells inside (e.g., core of) the tumor as compared to CD8+ T cells outside the tumor (e.g., peripheries of the tumor, e.g., the external perimeters of a tumor and/or near the vicinity of vasculatures of a tumor, e.g., in the tumor margin and/or stroma) may be identified as an immune-excluded tumor. In some embodiments, an immune-excluded tumor comprises less than 5%, less than 10%, or less than 15% CD8+ T cells inside the tumor and/or inside one or more tumor nests and greater than 5%, greater than 10%, or greater than 15% CD8+ T cells outside the tumor and/or outside one or more tumor nests. In some embodiments, an immune-excluded tumor comprises less than 5% CD8+ T cells inside the tumor and/or inside one or more tumor nests and greater than 5% CD8+ T cells outside of the tumor and/or outside one or more tumor nests. In some embodiments, an immune-excluded tumor comprises less than 10% CD8+ T cells inside the tumor and/or inside one or more tumor nests and greater than 10% CD8+ T cells outside of the tumor and/or outside one or more tumor nests. In some embodiments, an immune-excluded tumor comprises less than 15% CD8+ T cells inside the tumor and/or inside one or more tumor nests and greater than 15% CD8+ T cells outside of the tumor and/or outside one or more tumor nests.

In some embodiments, a tumor with higher levels of CD8+ T cells inside the tumor as compared to CD8+ T cells outside the tumor (e.g., the external perimeters of a tumor and/or near the vicinity of vasculatures of a tumor, e.g., in the tumor margin and/or stroma) may be identified as an immune-inflamed (or immune-infiltrated) tumor. In some embodiments, an immune-inflamed (or immune-infiltrated) tumor comprises greater than 5% CD8+ T cells inside the tumor. In some embodiments, an immune-inflamed (or immune-infiltrated) tumor comprises greater than 10% CD8+ T cells inside the tumor and/or inside one or more tumor nests. In some embodiments, an immune-inflamed (or immune-infiltrated) tumor comprises greater than 15% CD8+ T cells inside the tumor and/or inside one or more tumor nests. While immune-infiltrated tumors may sometimes have limited benefit from TGFβ inhibitor therapy, some immune-infiltrated tumors may nevertheless benefit from TGFβ inhibitor therapy, particularly when the tumor is also resistant or refractory to a checkpoint inhibitor therapy. Such tumors may exhibit an immunosuppressive phenotype. Such tumors may additionally or alternatively comprise infiltrated CD8+ cells that have reduced cytotoxic function, e.g., the CD8+ cells express reduced amounts of cytotoxic enzymes, such as perforin and/or granzyme B.

In some embodiments, a tumor with low levels of CD8+ T cells both inside and outside the tumor may be identified as an immune desert tumor. In some embodiments, an immune desert tumor comprises less than 5% CD8+ T cells inside the tumor and less than 10% CD8+ T cells in the tumor margin and/or stroma. In some embodiments, an immune desert tumor comprises less than 5% CD8+ T cells inside the tumor (and/or inside one or more tumor nests) and less than 5% CD8+ T cells in the tumor margin and/or stroma.

In some embodiments, CD8+ content in tumor compartments may be determined based on any of the methods described in Ziai et al. (PloS One. 2018; 13(1): e0190158), Massi et al. (J Immunother Cancer. 2019 Nov. 15; 7(1):308), Sharma et al. (Proc Natl Acad Sci USA. 2007 Mar. 6; 104(10):3967-72), or Echarti et al. (Cancers (Basel). 2019 September; 11(9): 1398), the contents of which are hereby incorporated in their entirety. In some embodiments, any of these methods may be used to determine the immune phenotype of the tumor.

In certain embodiments, the immune phenotype of a subject's cancer may be determined by average percent CD8 positivity (i.e., percentage of CD8+ lymphocytes) as measured over multiple (e.g., at least 5, at least 15, at least 25, at least 50, or more) tumor nests of a tumor (e.g., in one or more tumor biopsy samples). In certain embodiments, the immune phenotype of a given tumor nest may be determined by comparing the CD8 positivity inside the tumor nest to the CD8 positivity outside the tumor nest (e.g., in the tumor nest margin and/or the tumor nest stroma). In certain embodiments, a tumor nest may be identified as immune inflamed if the CD8 positivity inside the tumor nest is greater than 5%. In certain embodiments, a tumor nest may be identified as immune excluded if the CD8 positivity inside the tumor nest is less than 5% and the CD8 positivity in the tumor nest margin is greater than 5%. In certain embodiments, a tumor nest may be identified as an immune desert if the CD8 positivity inside the tumor nest is less than 5% and CD8 positivity in the tumor nest margin is less than 5%. In certain embodiments, a subject's cancer may be identified immune inflamed if greater than 50% of the total tumor area analyzed comprises tumor nests exhibiting immune inflamed phenotype. In certain embodiments, a subject's cancer may be identified as immune excluded if greater than 50% of the total tumor area analyzed comprises tumor nests exhibiting immune excluded phenotype. In certain embodiments, a subject's cancer may be identified as an immune desert if greater than 50% of the total tumor area analyzed comprises tumor nests exhibiting immune desert phenotype. In certain embodiments, a subject's cancer may be identified based on determination of CD8 positivity from more than one sample (e.g., at least three samples, e.g., four samples) taken from the same tumor.

In certain embodiments, a patient treated with a TGFβ inhibitor, e.g., a TGFβ inhibitor disclosed herein, e.g., in conjunction with a second therapy, has an immune infiltrated phenotype. In certain embodiments, the immune phenotype of a subject's cancer is evaluated to determine the ratio of Treg/CD8+ T cell in the TME. In certain embodiments, the patient has an immune-infiltrated phenotype. In certain embodiments, a patient has a high Treg/CD8+ T cell ratio in the TME and/or has infiltrated CD8+ T cells expressing decreased levels of cytotoxic enzymes (such as perforin and granzyme A/B) and/or proinflammatory cytokines (such as IFNγ). In certain embodiments, levels of circulating MDSCs are determined in the patient (in lieu of or in conjunction with measuring a Treg/CD8+ T cell ratio in a TME). In certain embodiments, the patient treated with a TGFβ inhibitor has an elevated number of circulating MDSCs. Such patient may not have responded to a prior treatment, e.g., to a prior checkpoint inhibitor treatment, and may be selected for a combination therapy (including a therapy comprising a checkpoint inhibitor) comprising administration one or more TGFβ inhibitors.

In certain embodiments, tumor biopsy samples may be obtained by core needle biopsy. In certain embodiments, three to five samples (e.g., four samples) may be taken from the same tumor. In certain embodiments, the needle may be inserted along a single trajectory, wherein multiple samples (e.g., three to five samples, e.g., four samples) may be taken at different tumors depths along the same needle trajectory. In certain embodiments, samples taken at different tumor depths may be used to analyze combined CD8 positivity over multiple tumor nests. In certain embodiments, the combined CD8 positivity determined in these samples may be representative of CD8 positivity in the rest of the tumor. In certain embodiments, the combined CD8 positivity determined in these samples may be used to identify immune phenotype of a subject's cancer.

In certain embodiments, the immune phenotype of a subject's tumor may be determined by combined analysis of the absolute number, percentage, ratio, and/or density of CD8+ cells in the tumor and the combined CD8 positivity (i.e., percentage of CD8+ lymphocytes) across tumor nests throughout the tumor.

In certain embodiments, tumor compartments may be identified, determined, and/or analyzed for markers such as CD8 content manually, e.g., by a pathologist inspection of tumor samples. In some embodiments, tumor compartments may be identified, determined, and/or analyzed for markers such as CD8 content by digital analysis, e.g., by using a software or computer program for automated identification. In certain embodiments, a skilled artisan may use such a software or computer program for automated identification of tumor nests and the boundaries between a tumor nest, stroma compartment, and/or tumor margin compartment. In certain embodiments, a software or computer program may be used to evaluate the distribution of suitable markers such as CD8+ T cells in the identified tumor nest, stromal compartment, and/or tumor margin compartment. In certain embodiments, the software or computer program may be based on one or more machine learning algorithms. In certain embodiments, the one or more machine learning algorithms may be based initially on manual classification of reference samples, e.g., by a trained pathologist. In some embodiments, the software or computer program may use a neural network approach with machine learning based on reference samples categorized manually, e.g., by a pathologist. Exemplary softwares or computer programs include any software or computer program that has the capability of intaking an image (e.g., microscope images of a tumor sample comprising immune staining), processing and analyzing the image, and segmenting the tumor compartments in the image based on specific parameters (e.g., nuclear staining, fibroblast staining, CD8+ staining, other biomarkers). In certain embodiments, the softwares or computer program may be any of those provided by Visiopharm, HALO (Indica Labs), CellProfiler Analyst, Aperio Image Analysis, Zeiss ZEN Intellesis, or ImageJ. Such programs may advantageously achieve sufficient resolution for visualizing certain characteristics of individual tumor nests within a solid tumor (e.g., boundaries for tumor nest, stroma, and/or margin compartments), as opposed to analyzing substantially the entire tumor as a whole.

In certain embodiments, a subject whose cancer exhibits an immune-inflamed phenotype but is not responsive to a checkpoint inhibitor therapy may be more responsive to a therapy comprising administration of a TGFβ inhibitor (e.g., Ab6). In some embodiments, such a subject is identified for treatment. In some embodiments, such a subject may have a high Treg/CD8+ T cell ratio in the tumor and/or have CD8+ T cells expressing decreased levels of cytotoxic enzymes and/or proinflammatory cytokines. In some embodiments, such subject may have an increased number of circulating MDSCs. In some embodiments, such a subject has a RCC such as ccRCC. In some embodiments, such a subject is administered a treatment comprising a TGF inhibitor, such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibodies that bind to αVβ1, αVβ3, αVβ5, αVβ6, αVβ8, α5β1, αIIbβ3, or α8β1 integrins, and inhibit downstream activation of TGFβ. E.g., selective inhibition of TGFβ1 and/or TGFβ3).

In certain embodiments, a subject whose cancer exhibits an immule-inflamed phenotype but is not responsive to a checkpoint inhibitor therapy may be more responsive to a combination therapy comprising a TGFβ inhibitor, such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ, e.g., selective inhibition of TGFβ1 and/or TGFβ3), and an additional cancer therapy, e.g., a checkpoint inhibitor. In some embodiments, the additional cancer therapy may comprise chemotherapy, radiation therapy (including radiotherapeutic agents), a cancer vaccine, or an immunotherapy comprising a checkpoint inhibitor such as an anti-PD-1, anti-PD-L1, or anti-CTLA-4 antibody. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181), and durvalumab (e.g., Imfinzi®). In certain embodiments, a subject whose cancer exhibits an immune-excluded phenotype is administered a combination therapy comprising a TGFβ inhibitor, such as a TGFβ1-selective inhibitor (e.g., Ab6), and an additional cancer therapy, e.g., a checkpoint inhibitor. In some embodiments, such a subject may have a high Treg/CD8+ T cell ratio in the tumor and/or have CD8+ T cells expressing decreased levels of cytotoxic enzymes and/or proinflammatory cytokines. In some embodiments, such a subject has a RCC such as ccRCC.

In certain embodiments, a subject whose cancer exhibits an immune-inflamed phenotype but is not responsive to a checkpoint inhibitor therapy may be more responsive to a combination therapy comprising a TGFβ inhibitor, such as a TGFβ1-selective inhibitor (e.g., Ab6), and a checkpoint inhibitor therapy (e.g., a PD1 or PDL1 antibody). In some embodiments, such a subject is identified for receiving the combination therapy. In some embodiments, such a subject is identified for receiving the combination therapy prior to receiving the checkpoint inhibitor therapy alone. In some embodiments, such a subject is identified for receiving the combination therapy prior to receiving either the checkpoint inhibitor therapy or the TGFβ inhibitor alone. In some embodiments, such a subject is treatment-naïve. In some embodiments, such a subject has previously received a checkpoint inhibitor therapy and is non-responsive to the checkpoint inhibitor therapy. In some embodiments, such a subject has cancer that exhibits an immune-excluded phenotype. In some embodiments, such a subject has previously received a checkpoint inhibitor therapy and is directly given a combination therapy (e.g., bypassing the need to first try treatment with a checkpoint inhibitor alone). In some embodiments, such a subject is administered a combination therapy comprising a TGFβ inhibitor, such as a TGFβ1-selective inhibitor (e.g., Ab6), and an additional cancer therapy, e.g., a PD1 or PDL1 antibody. In some embodiments, such a subject may have a high Treg/CD8+ T cell ratio in the tumor and/or have CD8+ T cells expressing decreased levels of cytotoxic enzymes and/or proinflammatory cytokines. In some embodiments, such a subject has a RCC such as ccRCC.

In some embodiments, a subject whose cancer exhibits an immune-inflamed phenotype but is not responsive to a checkpoint inhibitor therapy may be selected for treatment and/or monitored during and/or after administration of the therapy comprising a TGFβ inhibitor, such as a TGFβ1-selective inhibitor (e.g., Ab6). In some embodiments, such a subject may have a high Treg/CD8+ T cell ratio in the tumor and/or have CD8+ T cells expressing decreased levels of cytotoxic enzymes and/or proinflammatory cytokines. In some embodiments, such a subject has a RCC such as ccRCC. In some embodiments, patient selection and/or treatment efficacy is determined by measuring the level of cytotoxic T cells (e.g., CD8+ T cells) inside the tumor as compared to the level of cytotoxic T cells (e.g., CD8+ T cells) outside the tumor (e.g., in the margin). In certain embodiments, an increase in the levels of tumor-infiltrating cytotoxic T cells (e.g., CD8+ T cells) inside the tumor relative to outside the tumor (e.g., margin and/or stroma) following administration of a TGFβ inhibitor therapy (e.g., Ab6), alone or in combination with an additional therapy (e.g., a checkpoint inhibitor therapy), may indicate a therapeutic response (e.g., anti-tumor response). For instance, an increase of at least 10%, 15%, 20%, 25%, or more in tumor-infiltrating cytotoxic T cell levels following TGFβ inhibitor treatment (e.g., Ab6) as compared to tumor-infiltrating cytotoxic T cell levels before the treatment may be indicative of therapeutic response (e.g., anti-tumor response). In some embodiments, an increase of at least 10%, 15%, 20%, 25%, or more in total tumor area comprising immune inflamed tumor nests may be indicative of therapeutic response. In some embodiments, levels of cytolytic proteins such as perforin or granzyme B or proinflammatory cytokines such as IFNγ expressed by the tumor-infiltrating cytotoxic T cells may also be measured to determine the activation status of the tumor-infiltrating cytotoxic T cells. In some embodiments, an increase of at least 1.5-fold, or 2-fold, or 5-fold, or more in cytolytic protein levels may be indicative of therapeutic response (e.g., anti-tumor response). In some embodiments, a change of at least a 1.5-fold, 2-fold, 5-fold, or 10-fold, or more increase in IFNγ levels may be indicative of a therapeutic response (e.g., anti-tumor response). In some embodiments, treatment is continued if an increase in tumor-infiltrating cytotoxic T cells (e.g., CD8+ T cells) is detected.

In certain embodiments, a subject whose cancer exhibits an immune-inflamed phenotype may be more responsive to a therapy comprising a checkpoint inhibitor without a TGFβ inhibitor than would a subject having an immune-excluded phenotype. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®). In certain embodiments, a subject whose cancer exhibits an immune-inflamed phenotype is administered a checkpoint inhibitor. In some embodiments, the subject whose cancer exhibits an immune-inflamed phenotype is resistant or refractory to the checkpoint inhibitor. The cancer may also exhibit an immunosuppressive phenotype. In these instances, the subject may be administered a TGFβ inhibitor, such as a TGFβ1-selective inhibitor (e.g., Ab6), optionally where the TGFβ inhibitor is administered as a combination therapy with a checkpoint inhibitor. The cancer may be RCC, such as ccRCC.

In certain embodiments, immune phenotyping of a subject's tumor may be determined from a tumor biopsy sample (e.g., core needle biopsy sample), for example histologically, using one or more parameters such as, but not limited to, distribution of cytotoxic T cells (e.g., CD8+ T cells), percentage of cytotoxic T cells (e.g., CD8+ T cells) in the tumor versus stromal compartment, and percentage of cytotoxic T cells (e.g., CD8+ T cells) in the tumor margin.

In certain embodiments, a sample may be analyzed for its distribution of cytotoxic T cells (e.g., CD8+ T cells) using a method such as CD8 immunostaining. In certain embodiments, the distribution of cytotoxic T cells (e.g., CD8+ T cells) may be relatively uniform (e.g., distribution is homogeneous throughout the sample, e.g., CD8 density across tumor nests have a variance of 10% or lower). In some embodiments, a tumor nest (or cancer nest) refers to a mass of cells extending from a common center of a cancerous growth. In some embodiments, a tumor nest may comprise cells interspersed in stroma. In some embodiments, a tumor nest comprises at least 250 cells and at least 500 μm². In certain embodiments, a sample, such as a sample with an even distribution of cytotoxic T cells (e.g., CD8 T cells), may be analyzed to determine the percentages of cytotoxic T cells (e.g., CD8+ T cells) in the tumor and in the stroma. In certain embodiments, a high percentage (e.g., greater than 5%) of cytotoxic T cells (e.g., CD8+ T cells) in the tumor and a low percentage (e.g., less than 5%) of cytotoxic T cells (e.g., CD8+ T cells) in the stroma may be indicative of an inflamed tumor phenotype. In certain embodiments, a low percentage of cytotoxic T cells (e.g., CD8+ T cells) in both the tumor and the stroma (e.g., combined tumor and stroma CD8 percentage of less than 5%) may be indicative of a poorly immunogenic tumor phenotype (e.g., an immune desert phenotype). In certain embodiments, a low percentage (e.g., less than 5%) of cytotoxic T cells (e.g., CD8+ T cell cells) in the tumor and a high percentage (e.g., greater than 5%) of cytotoxic T cells (e.g., CD8+ T cell cells) in the stroma may be indicative of an immune-excluded tumor phenotype. In certain embodiments, a tumor-to-stroma CD8 ratio may be determined by dividing CD8 percentage in the tumor over the percentage in the stroma. In certain embodiments, a tumor-to-stroma CD8 ratio of greater than 1 may be indicative of an inflamed tumor phenotype. In certain embodiments, a tumor-to-stroma CD8 ratio of less than 1 may be indicative of an immune-excluded tumor. In certain embodiments, percentages of cytotoxic T cells may be determined by immunohistochemical analysis of CD8 immunostaining.

In certain embodiments, a sample, such as a sample with uneven distribution of cytotoxic T cells (e.g., CD8 density across tumor nests have a variance of greater than 10%), may be analyzed to determine the margin-to-stroma CD8 ratio. In certain embodiments, such ratio may be calculated by dividing CD8 density in the tumor margin over CD8 density in the tumor stroma. In certain embodiments, an immune excluded tumor exhibits a margin-to-stroma CD8 ratio of greater than 0.5 and less than 1.5.

In certain embodiments, a sample having a margin-to-stroma CD8 ratio of greater than 1.5 may be further analyzed to determine and/or confirm immune phenotyping (e.g., to determine and/or confirm whether the tumor has an immune-excluded phenotype) by evaluating tumor depth. In certain embodiments, tumor depth may be measured in increments of 20 μm-200 μm (e.g., 100 μm). In certain embodiments, tumor depth may be determined by pathological analysis and/or digital image analysis. In certain embodiments, a significant tumor depth may be indicated by a distance of about 2-fold or greater than the depth of the tumor margin. In certain embodiments, a tumor sample may have a tumor margin depth of 100 μm and a tumor depth measurement of greater than 200 μm, such sample would have a tumor depth score of greater than 2, and would therefore have significant tumor depth. In certain embodiments, significant tumor depth may be indicated by a ratio of 2 or greater as determined by dividing tumor depth by the depth of the tumor margin. In certain embodiments, tumor depth may be measured in increments corresponding to the depth of the tumor margin. For instance, the tumor depth of a tumor nest having a tumor margin of 100 μm may be measured in increments of 100 μm. In certain embodiments, a tumor sample with significant tumor depth may exhibit shallow penetration by cytotoxic T cells (e.g., the tumor sample having greater than 5% CD8 T cells but does not exhibit tumor penetration beyond one tumor depth increment). In certain embodiments, a tumor sample with significant tumor depth that exhibits shallow CD8 penetration may be indicative of an immune excluded tumor.

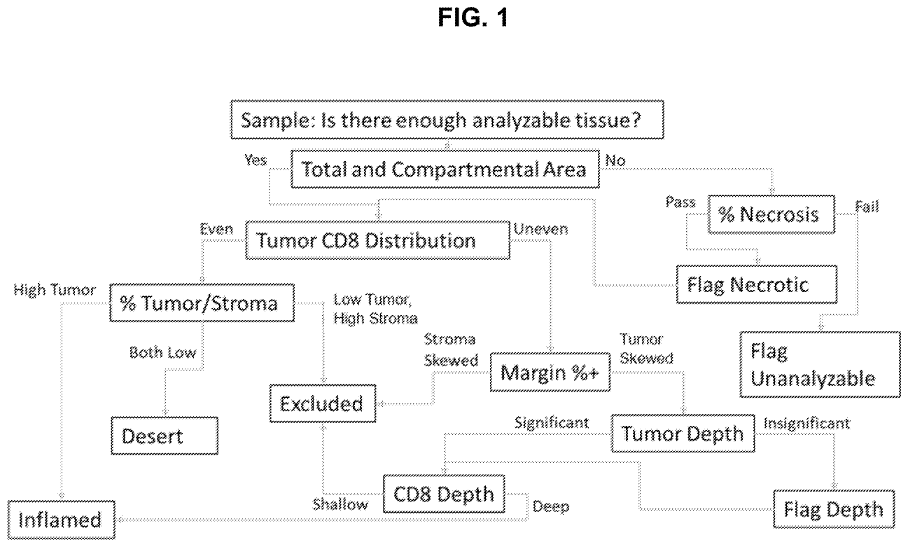

In certain embodiments, a tumor phenotype analysis may be conducted according to any part of the exemplary flow chart shown in FIG. 1, e.g., using all the steps in that figure.

In certain embodiments, a subject whose cancer exhibits an immune excluded phenotype may be selected for TGFβ inhibitor therapy (e.g., a TGFβ1 inhibitor such as Ab6). In certain embodiments, a subject whose cancer exhibits an immune excluded phenotype may be more responsive to a TGFβ inhibitor therapy (e.g., a TGFβ1 inhibitor such as Ab6). In certain embodiments, a subject whose cancer exhibits an immune-excluded phenotype may be more responsive to a combination therapy comprising a TGFβ inhibitor, such as a TGFβ1-selective inhibitor (e.g., Ab6), and a second cancer therapy, e.g., a checkpoint inhibitor therapy (e.g., a PD1 or PDL1 antibody).

In certain embodiments, a response to TGFβ inhibitor therapy (e.g., a TGFβ1 inhibitor such as Ab6) may be monitored and/or determined using parameters such as any of the ones described above. In certain embodiments, a change in a distribution of cytotoxic T cells (e.g., CD8+ T cells) in a pre-treatment tumor sample as compared to a corresponding post-treatment sample from the corresponding tumor may be indicative of a therapeutic response to treatment. In certain embodiments, a change (e.g., increase) of at least 1-fold (e.g., 1.1-fold, 1.2-fold, 1.3-fold, 1.4-fold, 1.5-fold, 1.6-fold, 1.7-fold, 1.8-fold, 1.9-fold, 2-fold, or greater) in the tumor-to-stroma CD8 density ratio between the pre-treatment and post-treatment tumor samples may be indicative of a therapeutic response. In certain embodiments, a change (e.g., increase) of 1.5-fold or greater in the tumor-to-stroma CD8 density ratio between the pre-treatment and post-treatment tumor samples may be indicative of a therapeutic response. In certain embodiments, the tumor-to-stroma CD8 density ratio may be determined by dividing CD8 cell density in the tumor nest over CD8 cell density in the tumor stroma. In certain embodiments, a change (e.g., increase) of 1.5-fold or greater in the density of cytotoxic T cells (e.g., CD8+ T cells) in the tumor margin between the pre-treatment and post-treatment tumor samples may be indicative of a therapeutic response. In certain embodiments, a change (e.g., increase) of 1.5-fold or greater in the tumor depth score of pre-treatment and post-treatment tumor samples may be indicative of a therapeutic response. In some embodiments, the TGFβ inhibitor therapy (e.g., a TGFβ1 inhibitor such as Ab6) achieves at least a 2-fold, e.g., 3-fold, 4-fold, 5-fold, 6-fold, 7-fold, 8-fold, 9-fold, 10-fold, 15-fold, 20-fold, or a greater degree of increase in the number of intratumoral T cells, e.g., when used in conjunction with a checkpoint inhibitor such as a PD-(L)1 antibody, relative to pre-treatment. In certain embodiments, treatment with a TGFβ inhibitor therapy (e.g., a TGFβ1 inhibitor such as Ab6), e.g., alone or in combination with one or more additional cancer therapies, may be continued if a therapeutic response is observed.

In certain embodiments, the pre-treatment and post-treatment samples have comparable tumor depth scores (e.g., variance of less than 0.25 in tumor depth scores of pre-treatment and post-treatment tumor samples) and the samples may be analyzed to determine therapeutic response according to one or more of the parameters described above. In certain embodiments, the pre-treatment and post-treatment samples have comparable total and compartmental areas (e.g., variance of less than 0.25 in analyzable total and compartmental area of pre-treatment and post-treatment tumor samples) and the samples may be analyzed to determine therapeutic response according to one or more of the parameters described above.

In some embodiments, percent necrosis in a tumor sample may be assessed by histological and/or digital image analysis, which may reflect the presence or activities of cytotoxic cells in the tumor. In some embodiments, percent necrosis in tumor samples may be compared in pre-treatment and post-treatment tumor samples collected from a subject administered a TGFβ inhibitor (e.g., Ab6). In some embodiments, increase of greater than 10% in percent necrosis (e.g., the proportion of necrotic area to total tissue area in a tumor sample) between pre-treatment and post-treatment samples may be indicative of a therapeutic response to TGFβ inhibitor therapy, e.g., TGFβ1 inhibitor such as Ab6. In some embodiments, an increase of 10% or greater in percent necrosis in or near the center of the tumor (e.g., the proportion of necrotic area inside the tumor margin) may be indicative of a therapeutic response.

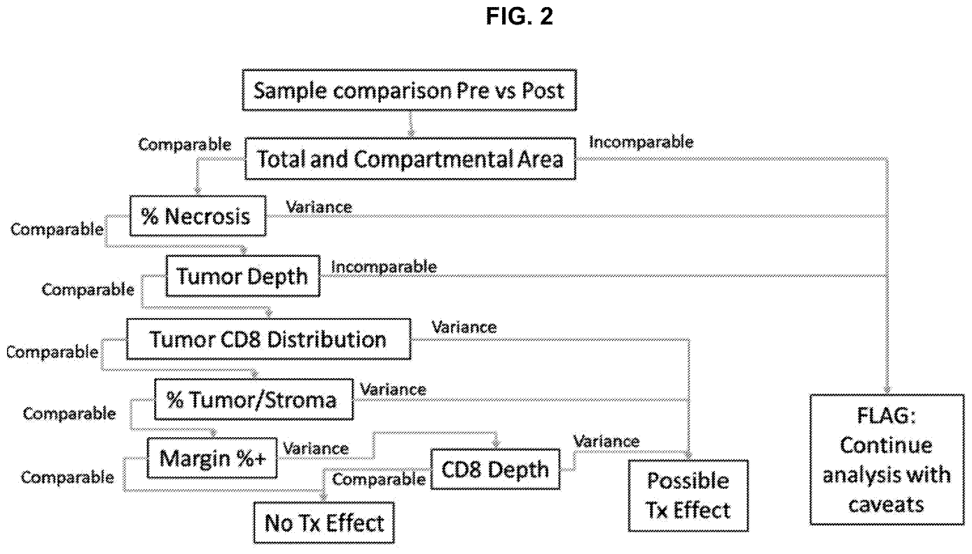

In certain embodiments, a therapeutic response may be determined according to any part of the exemplary flow chart shown in FIG. 2.

In some embodiments, an increased level of tumor-infiltrating cytotoxic T cells (e.g., CD8+ T cells), especially activated cytotoxic T cells, following TGFβ inhibitor therapy (e.g., a TGFβ1 inhibitor such as Ab6) may indicate conversion of an immune-excluded tumor microenvironment toward an immune-infiltrated or “inflamed” microenvironment. For instance, an increase of at least 1%, 2%, 3%, 4%, 5%, 6%, 7%, 8%, 9%, 10%, 15%, 20%, 25%, or more in tumor-associated cytotoxic T cell levels following TGFβ inhibitor treatment (e.g., Ab6) as compared to tumor-associated cytotoxic T cell levels before the treatment may be indicative of a reduction or reversal of immune suppression in the cancer. In some embodiments, an increase of at least 1%, 2%, 3%, 4%, 5%, 6%, 7%, 8%, 9%, 10%, 15%, 20%, 25%, or more in tumor area comprising immune inflamed tumor nests may be indicative of a reduction or reversal of immune suppression in the cancer. In some embodiments, levels of cytolytic proteins such as perforin or granzyme B or proinflammatory cytokines such as IFNγ expressed by the tumor-associated cytotoxic T cells may be measured to determine the activation status of the tumor-associated cytotoxic T cells. In some embodiments, an increase of at least 1-fold, 1.1-fold, 1.2-fold, 1.3-fold, 1.4-fold, 1.5-fold, or 2-fold, or 5-fold, or more in cytolytic protein levels may be indicative of reduction or reversal of immune suppression in the cancer. In some embodiments, a change of at least a 1.5-fold, 2-fold, 5-fold, or 10-fold, or more increase in IFNγ levels may be indicative of a reduction or reversal of immune suppression in the cancer. In some embodiments, treatment with the TGFβ inhibitor therapy (e.g., a TGFβ1 inhibitor such as Ab6) is continued if such a reduction or reversal of immune suppression in the cancer is detected.

Immunosuppressive lymphocytes associated with TMEs include TAMs and MDSCs. A significant fraction of tumor-associated macrophages is of so-called “M2” type, which has an immunosuppressive phenotype. Most of these cells are monocyte-derived cells that originate in the bone marrow. Intratumoral (e.g., tumor-associated) levels of immunosuppressive cells such as TAMs and MDSCs may also be measured to determine the status of immune suppression in a cancer. In some embodiments, a decrease of at least 10%, 15%, 20%, 25%, or more in the level of TAMs may be indicative of reduced or reversal of immune suppression. In certain embodiments, tumor-associated immune cells may be measured from a biopsy sample from the subject prior to and following TGFβ inhibitor treatment (e.g., Ab6). In certain embodiments, biopsy samples may be obtained between 28 days and 130 days following treatment administration.

In some carcinomas, regulatory T cells (Treg) and platelets are enriched in TMEs, possibly contributing to immunosuppressive phenotype (Liu et al., Front Immunol, 2022 Jun. 24, 13:791158). Since Tregs can suppress effector T cells, this may be one of the reasons why CD8+ T cell-infiltrated tumors can be resistant to cancer therapy.

The concept of “immune contexture” examines the TME from the perspective of tumor-infiltrating lymphocytes (i.e., tumor immune microenvironment or TIME). Tumor immune contexture refers to the localization (e.g., spatial organization) and/or density of the immune infiltrate in the TME. TIME is usually associated with the clinical outcome of cancer patients and has been used for estimating cancer prognosis (see, for example, Fridman et al., (2017) Nat Rev Clin Oncol. 14(12): 717-734) “The immune contexture in cancer prognosis and treatment”). Typically, tissue samples from tumors are collected (e.g., biopsy such as core needle biopsy) for TIL analyses. In some embodiments, TILs are analyzed by FACS-based methods. In some embodiments, TILs are analyzed by immunohistochemical (IHC) methods. In some embodiments, TILs are analyzed by so-called digital pathology (see, for example, Saltz et al., (2018) Cell Reports 23, 181-193. “Spatial organization and molecular correlation of tumor-infiltrating lymphocytes using deep learning on pathology images.”); (Scientific Reports 9: 13341 (2019) “A novel digital score for abundance of tumor infiltrating lymphocytes predicts disease free survival in oral squamous cell carcinoma”). In some embodiments, tumor biopsy samples may be used in various DNA- and/or RNA-based assays (e.g., RNAseq or Nanostring) to evaluate the tumor immune contexture. Without wishing to be bound by theory, it is possible that a reduction or reversal of immune suppression in a cancer/tumor, as indicated by increased cytotoxic T cells and decreased TAMs, may be predictive of therapeutic efficacy in subjects administered with TGFβ inhibitor alone (e.g., Ab6) or in conjunction with a checkpoint inhibitor therapy.

### Circulating/Circulatory Latent-TGFβ

According to the present disclosure, circulating latent TGFβ may serve as a target engagement biomarker. Where an activation inhibitor is selected as a therapeutic candidate, for example, such biomarker may be employed to evaluate or confirm in vivo target engagement by monitoring the levels of circulating latent TGF beta before and after administration. In some embodiments, circulating TGFβ1 in a blood sample (e.g., plasma and/or serum) comprises both latent and mature forms, the former of which representing vast majority of circulatory TGFβ1. In some embodiments, total circulating TGFβ (e.g., total circulating TGFβ1) may be measured, i.e., comprising both latent and mature TGFβ, for example by using an acid treatment step to liberate the mature growth factor (e.g., TGFβ1) from its latent complex and detecting with an enzyme-linked immunosorbent assay (ELISA) assay. In some embodiments, reagents such as antibodies that specifically bind the latent form of TGFβ (e.g., TGFβ1) may be employed to specifically measure circulatory latent TGFβ1. In some embodiments, a majority of the measured circulating TGFβ (e.g., circulating TGFβ1) is released from a latent complex. In some embodiments, the total circulating TGFβ (e.g., circulating TGFβ1) measured is equivalent to dissociated latent TGFβ (e.g., latent TGFβ1) in addition to any free TGFβ (e.g., TGFβ1) present prior to acid treatment, which is known to be only a small fraction of circulating TGFβ1. In some embodiments, only circulating latent TGFβ (e.g., circulating latent TGFβ1) is detectable. In some embodiments, circulating latent TGFβ (e.g., circulating latent circulating TGFβ1) is measured.

In some embodiments, circulating TGFβ (e.g., circulating latent TGFβ1) can be measured from a blood sample by any of the methods described in or adapted from Mussbacher et al., PLos One. 2017 Dec. 8; 12(12):e0188921 and Mancini et al. Transl Res. 2018 February; 192: 15-29, the contents of which are hereby incorporated by reference in their entirety.

Challenges associated with determining blood/serum TGFβ levels with accuracy have been well recognized. Platelets are a main source of TGFβ1 in circulation, and even moderate handling of blood samples, such as blood collection, pipetting of blood samples, mechanical agitation, etc., is known to cause the release of TGFβ1 from platelets in the sample, resulting in skewed readout.

Improved assays for measuring circulatory TGFβ levels comprising a sample collection step, sample processing step and measuring step, are disclosed in WO 2021/142448 and WO 2022/256723, which are incorporated herein by reference in their entirety.

In various embodiments, the present disclosure provides methods of determining and monitoring the level of circulating latent TGFβ in a sample obtained from a patient, such that unwanted or inadvertent TGFβ activation associated with sample processing and preparation is reduced. In certain embodiments, the methods disclosed herein may be used to determine or monitor the level of circulating latent TGFβ1, e.g., by using sample collection methods disclosed herein and/or by normalizing to control markers of platelet activation during collection, e.g., PF4 levels.

Following the sample processing step described above, the resulting samples (e.g., PPP et al.) may be used to carry out one or more measuring steps for circulatory TGFβ. Accordingly, the present disclosure provides, in various embodiments, a method for measuring circulating TGFβ levels in a blood sample, wherein the method comprises a collection step and a processing step, each of which is carried out at 2-8° C. using a CTAD collection tube. The processing step may comprise two centrifugation steps as described above, to generate a PPP fraction from the blood sample. The resulting PPP is used to measure TGFβ levels. In some embodiments, total TGFβ levels, which include both the active and latent TGFβ forms, are measured. In some embodiments, active TGFβ (mature growth factor) levels are measured. In some embodiments, latent TGFβ levels are measured. In some embodiments, a majority of the TGFβ measured in an acidified sample is from circulating latent TGFβ. In some embodiments, the level of the TGFβ1 isoform is selectively measured. In some embodiments, the measuring step may include acidification of the sample to release TGFβ (i.e., mature growth factor) from the latent complex (i.e., proTGFβ, such as proTGFβ1). ELISA-based methods may be employed to then capture and detect/quantitate TGFβ present in the sample.

Such assay steps may be incorporated in a treatment regimen for a patient. For example, such assays may be used for providing information to aid prognosis, diagnosis, target engagement, monitoring therapeutic response, etc.

In some embodiments, circulatory TGFβ levels may serve as a predictive biomarker.

In some embodiments, circulatory TGFβ levels may serve as a predictive biomarker for therapeutic response to a checkpoint inhibitor therapy. In some embodiments, high baseline levels of circulatory TGFβ levels (e.g., in the plasma) may be predictive of poor therapeutic response to a checkpoint inhibitor therapy (e.g., pembrolizumab) (Feun et al. Cancer. 2019 Oct. 15; 125(20):3603-3614).

Accordingly, in various embodiments, the treatment regimen may include administration of a therapy that includes a TGFβ inhibitor, such as TGFβ1 inhibitor. The TGFβ inhibitors include, for example, monoclonal antibodies that bind the latent form of TGFβ (i.e., proTGFβ, such as proTGFβ1) thereby preventing the release of the growth factor, such as Ab6 and other antibodies that work by the same mechanism of action (see, for example, WO 2000/014460, WO 2000/041390, PCT/2021/012930, WO 2018/013939, WO 2020/160291, WO 2021/039945). The TGFβ inhibitors include neutralizing antibodies and engineered constructs that incorporate an antigen-binding fragment thereof. Examples of neutralizing antibodies include GC1008 and its variants, and NIS-793 (XOMA089). The TGFβ inhibitors also include so-called ligand traps, which comprise the ligand binding fragment(s) of the TGFβ receptor(s). Examples of ligand traps include M7824 (bintrafusp alpha) and AVID200. The TGFβ inhibitors also include low molecular weight receptor kinase inhibitors, such as ALK5 inhibitors.

In various embodiments, the patient being administered the treatment regimen is diagnosed with, at risk of developing, or suspected to have a TGFβ-related disease, such as cancer, myeloproliferative disorders (such as myelofibrosis), fibrosis and immune disorders. Thus, in some embodiments, the present disclosure provides a TGFβ inhibitor for use in the treatment of a TGFβ-related disease in a subject, wherein the treatment comprises administration of a composition comprising a TGFβ inhibitor in an amount sufficient to treat the disease, wherein the treatment further comprises determination of circulatory TGFβ levels in accordance with the disclosure herein. In some embodiments, the treatment further comprises determination of circulatory MDSCs. In some embodiments, circulatory MDSC levels are determined by measuring cell-surface marker(s). In some embodiments, the cell-surface marker is LRRC33. In some embodiments, the patient is a cancer patient, wherein optionally the cancer comprises a solid tumor, such as locally advanced or metastatic tumor. In some embodiments, the patient previously received a cancer therapy, wherein the cancer therapy is checkpoint inhibitor, radiation therapy and/or chemotherapy. In some embodiments, the subject was unresponsive or refractory to the cancer therapy, wherein optionally the cancer therapy comprises a checkpoint inhibitor (e.g., checkpoint inhibitor-resistant). In some embodiments, the tumor is refractory to the cancer therapy. In some embodiments, the patient is naïve to a cancer therapy, e.g., a checkpoint inhibitor (i.e., a checkpoint inhibitor-naïve patient). In some embodiments, the checkpoint inhibitor-naïve patient is diagnosed with a type of cancer that has statistically shown to have low response rates (e.g., below 30%, below 25%, below 20%, below 15%, etc.) to checkpoint inhibitors, such as anti-PD-(L)1. In some embodiments, the solid tumor has an immune excluded phenotype and/or exhibits an immunosuppressive phenotype. In some embodiments, the solid tumor has low expression of PD-L1.

In various embodiments, the present disclosure provides methods of treating a TGFβ-related disorder, comprising monitoring the level of circulating TGFβ, e.g., circulating latent TGFβ (e.g., TGFβ1) in a sample obtained from a patient (e.g., in the blood, e.g., plasma and/or serum, of a patient) receiving a TGFβ inhibitor. In certain embodiments, circulating TGFβ, e.g., circulating latent TGFβ (e.g., TGFβ1) may be measured in plasma samples collected from the subject. In certain embodiments, measuring TGFβ, e.g., circulating latent TGFβ (e.g., TGFβ1) from the plasma may reduce the risk of inadvertently activating TGFβ, such as that observed during serum preparations and/or processing. Accordingly, the present disclosure includes a TGFβ inhibitor for use in the treatment of diseases such as cancer, myelofibrosis, and fibrosis, in a subject, wherein the treatment comprises a step of measuring circulating TGFβ levels from a plasma sample collected from the subject. Such samples may be collected before and/or after administration of a TGFβ inhibitor to treat such diseases.

The level of circulating latent TGFβ may be monitored alone or in conjunction with one or more of the biomarkers disclosed herein (e.g., MDSCs). In certain embodiments, the TGFβ inhibitor may be administered alone or in conjunction with an additional cancer therapy. In some embodiments, the treatment may be administered to a subject afflicted with a TGFβ-related cancer or myeloproliferative disorder. In some embodiments, the TGFβ inhibitor is a TGFβ1-selective antibody or antigen-binding fragment thereof encompassed in the current disclosure (e.g., Ab6). In some embodiments, the TGFβ inhibitor is an isoform-non-selective TGFβ inhibitor (such as low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors). In some embodiments, the TGFβ inhibitor is an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. E.g., selective inhibition of TGFβ1 and/or TGFβ3). In some embodiments, the additional cancer therapy may comprise chemotherapy, radiation therapy (including radiotherapeutic agents), a cancer vaccine, or an immunotherapy, such as a checkpoint inhibitor therapy, e.g., an anti-PD-1, anti-PD-L1, or anti-CTLA-4 antibody. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®).

In various embodiments, circulating latent TGFβ (e.g., circulating latent TGFβ1) may be measured in a sample obtained from a subject (e.g., whole blood or a blood component). In various embodiments, the circulating latent TGFβ levels (e.g., latent TGFβ1) may be measured within 1, 2, 3, 4, 5, 6, 7, 8, 10, 12, 14, 16, 18, 21, 22, 25, 28, 30, 35, 40, 45, 48, 50, or 56 days following administration of the TGFβ inhibitor to a subject, e.g., up to 56 days after administration of a therapeutic dose of a TGFβ inhibitor. In various embodiments, the circulating latent TGFβ levels (e.g., circulating latent TGFβ1) may be measured about 8 to about 672 hours following administration of a therapeutic dose of a TGFβ inhibitor. In various embodiments, the circulating latent TGFβ levels (e.g., circulating latent TGFβ1) may be measured about 72 to about 240 hours (e.g., about 72 to about 168 hours, about 84 to about 156 hours, about 96 to about 144 hours, about 108 to about 132 hours) following administration of a therapeutic dose of a TGFβ inhibitor. In various embodiments, the circulating latent TGFβ levels (e.g., circulating latent TGFβ1) may be measured about 120 hours following administration of a therapeutic dose of a TGFβ inhibitor. In some embodiments, the circulating latent TGFβ levels (e.g., circulating latent TGFβ1) may be measured by any method known in the art (e.g., ELISA). In preferred embodiments, circulating TGFβ levels are measured from a blood sample (e.g., a plasma sample).

In various embodiments, the present disclosure encompasses a method of treating cancer in a subject, wherein the treatment comprises determining a level of circulating TGFβ in the subject prior to administering a TGFβ inhibitor, administering to the subject a therapeutically effective amount of the TGFβ inhibitor, and determining a level of circulating TGFβ in the subject after administration. In some embodiments, the circulating TGFβ level is determined or has been determined by processing a blood sample from the subject below room temperature in a sample tube coated with an anticoagulant.

In various embodiments, a method of treating a cancer or other TGF-related disorder comprises administering a TGFβ inhibitor (e.g., an anti-TGFβ1 antibody) to a patient in need thereof and confirming the level of target engagement by the inhibitor. In some embodiments, determining the level of target engagement comprises determining the levels of circulating latent TGFβ (e.g., circulating latent TGFβ1) in a sample obtained from a patient (e.g., in the blood or a blood component of a patient) receiving the TGFβ inhibitor. In some embodiments, an increase in circulating latent TGFβ (e.g., circulating latent TGFβ1) after administration of the TGF inhibitor indicates target engagement. In some embodiments, the present disclosure provides a method of determining targeting engagement in a subject having cancer, comprising determining a level of circulating TGFβ in the subject prior to administering a TGFβ inhibitor, administering to the subject a therapeutically effective amount of the TGFβ inhibitor, and determining a level of circulating TGFβ in the subject after administration. In some embodiments, an increase in circulating TGFβ levels (e.g., circulating latent TGFβ1 levels) after administration as compared to before administration indicates target engagement of the TGFβ inhibitor. In some embodiments, an increase in circulating latent TGFβ (e.g., circulating latent TGFβ1) of at least 1.5-fold, at least 2-fold, at least 2.5-fold, at least 3-fold, at least 4-fold, at least 5-fold, at least 6-fold, at least 7-fold, at least 8-fold, at least 9-fold, at least 10-fold, or more, after administration of the TGF inhibitor indicates target engagement. In some embodiments, the circulating TGFβ levels are determined or have been determined by processing a blood sample from the subject below room temperature in a sample tube coated with an anticoagulant. In some embodiments, further therapeutically effective amount of the TGFβ inhibitor are administered if target engagement is detected.

In various embodiments, the present disclosure also provides methods of using circulating latent TGFβ levels (e.g., circulating latent TGFβ1 levels) to predict therapeutic response, as well as for informing further treatment decisions (e.g., by continuing treatment if an increase is observed). In some embodiments, an additional dose of the TGFβ inhibitor (e.g., an anti-TGFβ1 antibody) is administered if target engagement is detected. In some embodiments, the method of determining therapeutic efficacy comprises determining a level of circulating TGFβ in the subject prior to administering a TGFβ inhibitor, administering to the subject a therapeutically effective amount of the TGFβ inhibitor, and determining a level of circulating TGFβ in the subject after administration. In preferred embodiments, circulating TGFβ levels are measured from a blood sample. In some embodiments, the circulating TGFβ levels are determined or have been determined by processing the blood sample from the subject below room temperature in a sample tube coated with an anticoagulant. In some embodiments, further therapeutically effective amount of the TGFβ inhibitor are administered if efficacy is detected.

In one aspect of the current disclosure, levels of circulating latent TGFβ are determined to inform treatment and predict therapeutic efficacy in subjects administered a TGFβ inhibitor such as a TGFβ1-selective inhibitor described herein. In certain embodiments, a TGFβ inhibitor (e.g., Ab6) is administered alone or concurrently (e.g., simultaneously), separately, or sequentially with an additional cancer therapy, e.g., a checkpoint inhibitor therapy, such that the amount of TGFβ1 inhibition administered is sufficient to increase the levels of circulating latent-TGFβ (e.g., circulating latent TGFβ1) as compared to baseline circulating latent-TGFβlevels. Circulating latent-TGFβlevels may be measured prior to or after each treatment such that an increase in circulating latent-TGFβlevels (e.g., latent TGFβ1) following the treatment indicates therapeutic efficacy. For instance, circulating latent-TGFβlevels (e.g., circulating latent TGFβ1) may be measured prior to and after the administration of a TGFβ inhibitor (e.g., Ab6) and an increase in circulating latent-TGFβlevels (e.g., latent TGFβ1) following the treatment predicts therapeutic efficacy. In some embodiments, treatment is continued if an increase is detected. In certain embodiments, circulating latent-TGFβlevels may be measured prior to and following administration of a first dose of a TGFβ inhibitor such as a TGFβ1 inhibitor described herein (e.g., Ab6), and an increase in circulating latent-TGFβ levels (e.g., circulating latent TGFβ1) following the administration predicts therapeutic efficacy and further warrants administration of a second or more dose(s) of the TGFβ inhibitor. In some embodiments, circulating latent-TGFβ levels (e.g., circulating latent TGFβ1) may be measured prior to and after a combination treatment of TGFβ inhibitor such as a TGFβ1-selective inhibitor (e.g., Ab6), and an additional therapy (e.g., a checkpoint inhibitor therapy), administered concurrently (e.g., simultaneously), separately, or sequentially, and a change in circulating latent-TGFβlevels following the treatment predicts therapeutic efficacy. In some embodiments, treatment is continued if an increase is detected. In some embodiments, an increase in circulating latent TGFβ (e.g., TGFβ1) of at least 1.5-fold, at least 2-fold, at least 2.5-fold, at least 3-fold, at least 4-fold, at least 5-fold, at least 6-fold, at least 7-fold, at least 8-fold, at least 9-fold, at least 10-fold, or more, after administration of the TGF inhibitor indicates therapeutic efficacy. In some embodiments, the increase in circulating latent-TGFβlevels following a combination treatment may warrant continuation of treatment. In preferred embodiments, circulating TGFβ levels are measured from a blood sample, wherein the blood sample is optionally processed below room temperature in a sample tube coated with an anticoagulant.

In certain embodiments, the current disclosure provides a method of treating a cancer in a subject, comprising administering a second dose of a TGFβ inhibitor to a subject having an elevated level of circulating TGFβ after receiving a first dose the TGFβ inhibitor, wherein the level of TGFβ has been measured by processing a blood sample from the subject below room temperature in a sample tube coated with an anticoagulant.

In certain embodiments, the current disclosure provides a method of treating a cancer in a subject comprising determining a level of circulating TGFβ in the subject prior to administering a TGFβ inhibitor, administering to the subject a first dose of TGFβ inhibitor, determining a level of circulating TGFβ in the subject after administration, and administering a second dose of the TGFβ inhibitor to the subject if the level of circulating TGFβ is elevated. In some embodiments, the level of TGFβ comprises processing a blood sample from the subject below room temperature in a sample tube coated with an anticoagulant. In some embodiments, the level of circulating TGFβ after the first dose of the TGFβ inhibitor is elevated by at least 1.5-fold, at least 2-fold, at least 2.5-fold, at least 3-fold, at least 4-fold, at least 5-fold, or more as compared the level of circulating TGFβ before the first dose of the TGFβ inhibitor.

In some embodiments, the cancer comprises a solid tumor, wherein optionally the solid tumor is selected from: melanoma (e.g., metastatic melanoma), triple-negative breast cancer, HER2-positive breast cancer, colorectal cancer (e.g., microsatellite stable-colorectal cancer), lung cancer (e.g., metastatic non-small cell lung cancer, small cell lung cancer), esophageal cancer, pancreatic cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer (e.g., gastric cancer), head and neck squamous cell cancer, urothelial carcinoma (e.g., metastatic urothelial carcinoma), hepatocellular carcinoma, or thyroid cancer.

In various embodiments, the current disclosure encompasses a method of treating a TGFβ-related disorder comprising administering a therapeutically effective amount of a TGFβ inhibitor to a subject having a TGFβ-related disorder, wherein the therapeutically effective amount is an amount sufficient to increase the level of circulating latent TGFβ (e.g., circulating latent TGFβ1). In certain embodiments, the TGFβ inhibitor is a TGFβ activation inhibitor. In certain embodiments, the TGFβ inhibitor is a TGFβ1 inhibitor (e.g., Ab6). In certain embodiments, the circulating latent TGFβ is latent TGFβ1. In some embodiments, the therapeutically effective amount of the TGFβ inhibitor (e.g., Ab6) is between 0.1-30 mg/kg per dose. In some embodiments, therapeutically effective amount of the TGFβ inhibitor (e.g., Ab6) is between 1-30 mg/kg per dose. In some embodiments, the therapeutically effective amount of the TGFβ inhibitor (e.g., Ab6) is between 5-20 mg/kg per dose. In some embodiments, the therapeutically effective amount of the TGFβ inhibitor (e.g., Ab6) is between 3-10 mg/kg per dose. In some embodiments, the therapeutically effective amount of the TGFβ inhibitor (e.g., Ab6) is between 1-10 mg/kg per dose. In some embodiments, the therapeutically effective amount of the TGFβ inhibitor (e.g., Ab6) is between 2-7 mg/kg per dose. In some embodiments, the therapeutically effective amount of the TGFβ inhibitor (e.g., Ab6) is about 2-6 mg/kg per dose. In some embodiments, the therapeutically effective amount of the TGFβ inhibitor (e.g., Ab6) is about 1 mg/kg per dose. In some embodiments, doses are administered about every three weeks. In some embodiments, the TGFβ inhibitor (e.g., Ab6) is dosed weekly, every 2 weeks, every 3 weeks, every 4 weeks, monthly, every 6 weeks, every 8 weeks, bi-monthly, every 10 weeks, every 12 weeks, every 3 months, every 4 months, every 6 months, every 8 months, every 10 months, or once a year. In preferred embodiments, circulating TGFβ levels are measured from a blood sample (e.g., a plasma sample, serum sample, etc.).

In various embodiments, total circulatory TGFβ1 (e.g., circulating latent TGFβ1) in blood samples collected from patients may range between about 2-200 ng/mL at baseline, although the measured amounts vary depending on the individuals, health status, and the exact assays being employed. In certain embodiments, total circulatory TGFβ1 (e.g., circulating latent TGFβ1) in blood samples collected from patients may range between about 1 ng/mL to about 10 ng (e.g., about 1000 μg/mL to about 7000 μg/mL). In certain embodiments, the level of circulating latent TGFβ (e.g., latent TGFβ1) following administration of a TGFβ inhibitor (e.g., Ab6) is increased by at least 1.5-fold (e.g., at least 1.5-fold, at least 2-fold, at least 3-fold, at least 4-fold, at least 5-fold, at least 6-fold, at least 7-fold, at least 8-fold, at least 9-fold, at least 10-fold, or more) as compared to circulating latent TGFβ levels prior to the administration. In preferred embodiments, circulating TGFβ levels are measured from a blood sample (e.g., a plasma sample, serum sample, etc.).

In certain embodiments, circulating latent TGFβ levels (e.g., latent TGFβ1) may be used to monitor target engagement and pharmacological activity of a TGFβ inhibitor in a subject receiving a TGFβ inhibitor therapy (e.g., a TGFβ activation inhibitor, e.g., Ab6). In certain embodiments, circulating latent TGFβ levels (e.g., latent TGFβ1 levels) may be measured prior to and after administration of a first dose of TGFβ inhibitor (e.g., Ab6) such that an increase of at least 1.5-fold (e.g., at least 1.5-fold, at least 2-fold, at least 3-fold, at least 4-fold, at least 5-fold, at least 6-fold, at least 7-fold, at least 8-fold, at least 9-fold, at least 10-fold, or more) in circulating latent TGFβ levels following the administration indicates target engagement (e.g., binding of the TGFβ inhibitor to human large latent proTGFβ1 complex). In certain embodiments, circulating latent TGFβ levels (e.g., latent TGFβ1) may be measured prior to and after administration of a first dose of TGFβ inhibitor (e.g., Ab6) such that an increase in circulating latent TGFβ levels (e.g., latent TGFβ1) following the administration indicates therapeutic efficacy. In certain embodiments, treatment is continued if an increase in circulating latent-TGFβlevels (e.g., latent TGFβ1) following administration of a TGFβ inhibitor (e.g., Ab6) is detected. In preferred embodiments, circulating TGFβ levels are measured from a blood sample (e.g., a plasma sample, serum sample, etc.).

In some embodiments, circulating latent-TGFβlevels (e.g., latent TGFβ1) may be measured prior to and after administration of a first dose of a TGFβ inhibitor (e.g., Ab6), and an increase in circulating latent-TGFβ levels (e.g., latent TGFβ1) after the administration indicates target engagement and/or treatment response, and/or further warrants administration of a second or more dose(s) of the TGFβ inhibitor. In another embodiment, circulating latent-TGFβlevels may be measured prior to and after administration of a first dose of a combination treatment comprising a checkpoint inhibitor therapy and a TGFβ inhibitor such as a TGFβ1-selective inhibitor (e.g., Ab6), and an increase in circulating latent-TGFβlevels after the administration indicates target engagement and/or treatment response, and/or further warrants continuation of treatment. In various embodiments, the combination therapy comprising a checkpoint inhibitor therapy and a TGFβ inhibitor such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). In preferred embodiments, circulating TGFβ levels are measured from a blood (e.g., plasma sample, serum sample, etc.).

In any of the preceding embodiments, circulating TGFβ is circulating TGFβ1. In various embodiments, the circulating TGFβ1 is measured from a blood sample collected from the subject. In various embodiments, the blood sample is processed below room temperature in a sample tube containing or coated with an anticoagulant.

### Immune Safety

Cytokines play an important role in normal immune responses, but when the immune system is triggered to become hyperactive, the positive feedback loop of cytokine production can lead to a “cytokine storm” or hypercytokinemia, a situation in which excessive cytokine production causes an immune response that can damage organs, especially the lungs and kidneys, and even lead to death. Such condition is characterized by markedly elevated proinflammatory cytokines in the serum. Historically, a Phase 1 Trial of the anti-CD28 monoclonal antibody TGN1412 in healthy volunteers led to a life-threatening “cytokine storm” response resulted from an unexpected systemic and rapid induction of proinflammatory cytokines (Suntharalingam G et al., N Engl J Med. 2006 Sep. 7; 355(10):1018-28). This incident prompted heightened awareness of the potential danger associated with pharmacologic stimulation of T cells.

Whilst TGFβ-directed therapies do not target a specific T cell receptor or its ligand, Applicant of the present disclosure reasoned that it was prudent to carry out immune safety assessment, including, for example, in vitro cytokine release assays, in vivo cytokine measurements from plasma samples of non-human primate treated with a TGFβ inhibitor, and platelet assays using human platelets.

In some embodiments, selection of a TGFβ inhibitor for therapeutic use and/or large-scale production thereof includes an assessment of the ability for the TGFβ inhibitor to trigger cytokine release from cytokine-producing cells. In such an assessment, one or more of the cytokines (e.g., inflammatory cytokines) IL-2, TNFα, IFNγ, IL-1p, CCL2 (MCP-1), and IL-6 may be assayed, e.g., by exposure to peripheral blood mononuclear cell (PBMC) constituents from heathy donors. Cytokine response after exposure to an antibody disclosed herein, e.g., Ab6, may be compared to release after exposure to a control, e.g., an IgG isotype negative control antibody, or any other suitable control depending on the TGFβ inhibitor being tested. Cytokine activation may be assessed in plate-bound (e.g., immobilized) and/or soluble assay formats. Levels of IFNγ, IL-2, IL-1p, TNFα, IL-6, and CCL2 (MCP-1) should not exceed 10-fold, e.g., 8-, 6-, 4-, or 2-fold the activation in the negative control. In some embodiments, a positive control may also be used to confirm cytokine activation in the sample, e.g., in the PBMCs. In some embodiments, these in vitro cytokine release results may be further confirmed in vivo, e.g., in an animal model such as a monkey toxicology study, e.g., a 4-week GLP repeat-dose monkey study.

Human platelets have been reported to express GARP, which can form TGFβ1 LLCs (Tran et al., 2009. Proc Natl Acad Sci USA. 106(32): 13445-13450). In some embodiments, an antibody disclosed herein, e.g., Ab6, does not significantly bind to and/or activate platelets. In some embodiments, platelet activation is evaluated in vitro. In some embodiments, platelet aggregation, binding, and activation may be assessed in human whole blood or platelet-rich plasma from healthy donors. Platelet aggregation and binding after exposure to an antibody disclosed herein, e.g., Ab6 may be compared to exposure to a negative control, e.g., saline solution, or a reference sample, e.g., a buffered solution. In selecting a suitable TGFβ inhibitor for therapeutic use, the candidate drug should be evaluated to ensure that it does not trigger spontaneous or agonist-induced activation. In addition, the drug should not interfere with the normal function of platelets (e.g., aggregation or clotting). In certain embodiments, platelet aggregation and binding do not exceed 10% above the aggregation in the negative control. In some embodiments, platelet activation following exposure to an antibody disclosed herein, e.g., Ab6, may be compared to exposure to a positive control, e.g., adenosine diphosphate (ADP). The activation status of platelets may be determined by surface expression of activation markers e.g., CD62P (P-Selectin) and GARP detectable by flow cytometry. Platelet activation should not exceed 10% above the activation in the negative control. In some embodiments, in vitro platelet response results may be further confirmed in vivo, e.g., in an animal model such as an immune-directed safety study in non-human primates, e.g., a 4-week GLP repeat-dose monkey study.

In some embodiments, selection of an antibody or an antigen-binding fragment thereof for therapeutic use may include: identifying an antibody or antigen-binding fragment that meets the criteria of one or more of those described herein; carrying out an in vivo efficacy study in a suitable preclinical model to determine an effective amount of the antibody or the fragment; carrying out an in vivo safety/toxicology study in a suitable model to determine an amount of the antibody that is safe or toxic (e.g., MTD, NOAEL, or any art-recognized parameters for evaluating safety/toxicity); and, selecting the antibody or the fragment that provides at least a three-fold therapeutic window (preferably 6-fold, more preferably a 10-fold therapeutic window, even more preferably a 15-fold therapeutic window). In certain embodiments, the in vivo efficacy study is carried out in two or more suitable preclinical models that recapitulate human conditions. In some embodiments, such preclinical models comprise a TGFβ1-positive cancer, which may optionally comprise an immunosuppressive tumor. The immunosuppressive tumor may be resistant to a cancer therapy such as CBT, chemotherapy and radiation therapy (including a radiotherapeutic agent). In some embodiments, the preclinical models are selected from MBT-2, Cloudman S91 and EMT6 tumor models. In some embodiments, such preclinical models comprise TGFβ1-positive fibrosis. In some embodiments, the preclinical models are selected from liver fibrosis model, kidney fibrosis model, lung fibrosis model, heart (cardiac) fibrosis model, skin fibrosis model.

Identification of an antibody or antigen-binding fragment thereof for therapeutic use may further include carrying out an immune safety assay, which may include, but is not limited to, measuring cytokine release and/or determining the impact of the antibody or antigen-binding fragment on platelet binding, activation, and/or aggregation. In certain embodiments, cytokine release may be measured in vitro using PBMCs or in vivo using a preclinical model such as non-human primates. In certain embodiments, the antibody or antigen-binding fragment thereof does not induce a greater than 10-fold release in IL-6, IFNγ, and/or TNFα levels as compared to levels in an IgG control sample in the immune safety assessment. In certain embodiments, assessment of platelet binding, activation, and aggregation may be carried out in vitro using PBMCs. In some embodiments, the antibody or antigen-binding fragment thereof does not induce a more than 10% increase in platelet binding, activation, and/or aggregation as compared to buffer or isotype control in the immune safety assessment.

The selected antibody or the fragment may be used in the manufacture of a pharmaceutical composition comprising the antibody or the fragment. Such pharmaceutical composition may be used in the treatment of a TGFβ indication in a subject as described herein. For example, the TGFβ indication may be a proliferative disorder, e.g., a TGFβ1-positive cancer or a fibrotic disorder, such as organ fibrosis. According to exemplary embodiments, the organ fibrosis can be pulmonary (lung) fibrosis. Thus, the invention includes a method for manufacturing a pharmaceutical composition comprising a TGFβ inhibitor, wherein the method includes the step of selecting a TGFβ inhibitor which is tested for immune safety as assessed by immune safety assessment comprising cytokine release assays and optionally further comprising a platelet assay. The TGFβ inhibitor selected by the method does not trigger unacceptable levels of cytokine release (e.g., no more than 10-fold, but more preferably within 2.5-fold as compared to control such as IgG control). Similarly, the TGFβ inhibitor selected by the method does not cause unacceptable levels of platelet aggregation, platelet activation and/or platelet binding. Such TGFβ inhibitor is then manufactured at large-scale, for example 250 L or greater, e.g., 1000 L, 2000 L, 3000 L, 4000 L or greater, for commercial production of the pharmaceutical composition comprising the TGFβ inhibitor.

Isoform Selectivity and Mechanisms of Action of TGFβ Inhibitors

TGFβ Inhibitors Useful for Carrying Out Various Embodiments of the Disclosure are Aimed to pharmacologically interfere with one or more aspects of TGFβ1 function in vivo. The TGFβ inhibitor may be a TGFβ1 inhibitor, such as a TGFβ1-isoform selective inhibitor, or an isoform-non-selective inhibitor. Isoform-non-selective inhibitors include, without limitation, low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors.

From a safety standpoint, there has been an increasing recognition that broad inhibition of TGFβ across isoforms may be a cause of observed toxicities, which underscores the fact that no TGFβ inhibitors have been successfully developed to this day. To circumvent potentially dangerous adverse effects, a number of groups have recently turned to identifying inhibitors that target a subset—but not all—of the isoforms and still retain efficacy. From an efficacy standpoint, however, the prevailing view of the field remains to be that it is advantageous to inhibit multiple isoforms of TGFβ to achieve therapeutic effects, and to accommodate this, toxicity management by “careful dosing regimen” is suggested as a solution (Brennan et al., (2018) mAbs, 10:1, 1-17). Consistent with this premise, numerous groups are developing TGFβ inhibitors that target more than one isoform. These include low molecular weight antagonists of TGFβ receptors, e.g., ALK5 antagonists, such as Galunisertib (LY2157299 monohydrate, Eli Lilly); monoclonal antibodies (such as neutralizing antibodies) that inhibit all three isoforms (“pan-inhibitor” antibodies) (see, for example, WO 2018/134681); monoclonal antibodies that preferentially inhibit two of the three isoforms (e.g., antibodies against TGFβ1/2 (for example, WO 2016/161410) and TGFβ1/3 (for example, WO 2006/116002 and WO 2020/051333); integrin inhibitors such as antibodies that bind to αᵥβ₃, αᵥβ₅, αᵥβ₆, αᵥβ₈, α₅β₁, α_{IIb}β₃, or α₈β₁ integrins and inhibit downstream activation of TGFβ, e.g., selective inhibition of TGFβ1 and/or TGFβ3 (e.g., PLN-74809, a small-molecule, dual selective inhibitor of αᵥβ₁/αᵥβ₆, Pliant Therapeutics, San Francisco, CA), and engineered molecules (e.g., fusion proteins) such as ligand traps (for example, see International Publication Nos. WO 2018/029367; WO 2018/129331 and WO 2018/158727). Similarly, inhibitors of integrins such as αᵥβ₆ also block integrin-dependent activation of both TGFβ1 and TGFβ3 and therefore may be considered as isoform-non-selective inhibitors of TGFβ signaling.

Previously, Applicant demonstrated that inhibition of TGFβ1 alone was sufficient to sensitize immunosuppressive tumors to a checkpoint inhibitor therapy even in tumors where both TGFβ1/3 are co-expressed (see International Publication No. WO/2020/014460). Similarly, TGFβ1-selective inhibitors are shown to mitigate fibrosis in preclinical models, including mouse liver fibrosis model where both of the TGFβ1/3 isoforms are co-expressed in the fibrotic tissue, albeit in discrete cell types (herein). Surprisingly, inhibition of TGFβ3 promoted pro-fibrotic phenotypes. The exacerbation of fibrosis is observed when the TGFβ3 inhibitor is used alone. In addition, when used in combination with a TGFβ1-selective inhibitor, the TGFβ3 inhibitor attenuated the anti-fibrotic effect of the TGFβ1-selective inhibitor, as evidenced by increased collagen accumulation in the fibrotic liver. These results raise the possibility that inhibitory potency against TGFβ3 may be an undesirable feature of TGFβ inhibitors to be used as therapy in situations where fibrosis is a concern. Similar pro-fibrotic concerns may also apply in other indications, including tumors such as RCC.

### Cancer/Malignancies

Various cancers involve TGFβ activities, e.g., TGFβ1 activities, and may be treated with the antibodies, compositions, and methods of the present disclosure. As used herein, the term “cancer” comprises any of various malignant neoplasms, optionally associated with TGFβ1-positive cells. Such malignant neoplasms are characterized by the proliferation of anaplastic cells that tend to invade surrounding tissue and metastasize to new body sites and also refers to the pathological condition characterized by such malignant neoplastic growths. The source of TGFβ1 may vary and may include the malignant (cancer) cells themselves, as well as their surrounding or support cells/tissues, including, for example, the extracellular matrix, various immune cells, and any combinations thereof.

Examples of cancer which may be treated in accordance with the present disclosure include but are not limited to, carcinoma, lymphoma, blastoma, sarcoma, and leukemia or lymphoid malignancies. More particular examples of such cancers include, but are not limited to, anal carcinoma; bile duct cancer; brain tumor (including glioblastoma); breast cancer, e.g., HER2+ breast cancer and triple-negative breast cancer (TNBC), ductal carcinoma in situ (DCIS); cervical cancer; colorectal cancer; endometrial or uterine carcinoma; esophageal cancer; gastric or gastrointestinal cancer; gastrointestinal carcinoid tumor; gastrointestinal stromal tumors (GIST); head and neck cancer, e.g., head and neck squamous cell cancer (HNSCC); liver cancer, e.g., hepatocellular carcinoma (HCC); lung cancer, including small-cell lung cancer (SCLC), non-small cell lung cancer (NSCLC), metastatic NSCLC, adenocarcinoma of the lung, and squamous carcinoma of the lung; melanoma; ovarian cancer; pancreatic cancer (e.g., pancreatic ductal adenocarcinoma (PDAC); penile carcinoma; prostate cancer, e.g., castration-resistant prostate cancer (CRPC); renal cell carcinoma (RCC), e.g., clear cell RCC; cancer of the peritoneum; salivary gland carcinoma; thyroid cancer; urothelial carcinoma (UC) of the bladder and urinary tract, including metastatic UC (mUC); urothelial bladder cancer, muscle-invasive bladder cancer (MIBC), and non-muscle-invasive bladder cancer (NMIBC); myeloproliferative neoplasms (MPN), including chronic myeloid leukemia (CML), polycythemia vera (PV), primary myelofibrosis (PMF), essential thrombocythemia (ET), chronic neutrophilic leukemia (CNL); myelodysplastic syndromes (MDS); myeloproliferative neoplasms (MDS/MPN); acute lymphoblastic leukemia (ALL); acute myeloid leukemia (AML); chronic lymphocytic leukemia (CLL); multiple myeloma; Hodgkin's lymphoma; non-Hodgkin's lymphoma (NHL), including diffuse large B cell lymphoma (DLBCL), follicular lymphoma, hairy cell leukemia; mantle cell lymphoma; monoclonal gammopathy of undetermined significance (MGUS); plasma cell myeloma; waldenstrom macroglobulinemia; and mature T and NK neoplasms. In certain embodiments, a cancer which may be treated in accordance with the present disclosure includes one having high tumor mutational burden.

Affirmative identification of cancer as “TGFβ1-positive” is not required for carrying out the therapeutic methods described herein but is encompassed in some embodiments. Typically, certain cancer types are known to be or suspected, based on credible evidence, to be associated with TGFβ1 signaling.

Cancers may be localized (e.g., solid tumors) or systemic. In the context of the present disclosure, the term “localized” (as in “localized tumor”) refers to anatomically isolated or isolatable abnormalities/lesions, such as solid malignancies, as opposed to systemic disease (e.g., so-called liquid tumors or blood cancers). Certain cancers, such as certain types of leukemia (e.g., myelofibrosis) and multiple myeloma, for example, may have both a localized component (for instance the bone marrow) and a systemic component (for instance circulating blood cells) to the disease. In some embodiments, cancers may be systemic, such as hematological malignancies. Cancers that may be treated according to the present disclosure are TGFβ1-positive and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. In some embodiments, the cancer may be an advanced cancer, such as a locally advanced solid tumor and metastatic cancer.

In some embodiments, the cancer may be a cancer having elevated TGFβ1 levels associated with reactive oxygen species (ROS). In some embodiments, the cancer may be a cancer having elevated ROS levels and expressing high levels of TGFβ1. TGFβ activation can be triggered by ROS (Jobling et al., 2006. Radiat Res. 166: 839-848). In some embodiments, a TGFβ1 inhibitor can be used to block TGFβ activation by ROS.

Antibodies or antigen-binding fragments thereof encompassed by the present disclosure may be used in the treatment of cancer, including, without limitation: myelofibrosis, melanoma, adjuvant melanoma, renal cell carcinoma (RCC), including clear cell RCC, papillary RCC, chromophobe RCC, collecting duct RCC, or unclassified RCC, bladder cancer, colorectal cancer (CRC) (e.g., microsatellite-stable CRC, mismatch repair deficient colorectal cancer), colon cancer, rectal cancer, anal cancer, breast cancer, triple-negative breast cancer (TNBC), HER2-negative breast cancer, HER2-positive breast cancer, BRCA-mutated breast cancer, hematologic malignancies, non-small cell carcinoma, non-small cell lung cancer/carcinoma (NSCLC), small cell lung cancer/carcinoma (SCLC), extensive-stage small cell lung cancer (ES-SCLC), lymphoma (classical Hodgkin's and non-Hodgkin's), primary mediastinal large B-cell lymphoma (PMBCL), T-cell lymphoma, diffuse large B-cell lymphoma, histiocytic sarcoma, follicular dendritic cell sarcoma, interdigitating dendritic cell sarcoma, myeloma, chronic lymphocytic leukemia (CLL), acute myeloid leukemia (AML), small lymphocytic lymphoma (SLL), head and neck cancer (e.g., head and neck squamous cell cancer), urothelial cancer e.g., metastatic urothelial carcinoma), merkel cell carcinoma (e.g., metastatic merkel cell carcinoma), merkel cell skin cancer, cancer with high microsatellite instability (MSI-H), cancer with mismatch repair deficiency (dMMR), tumor mutation burden high cancer, mesothelioma (e.g., malignant pleural mesothelioma), gastric cancer, gastroesophageal junction cancer (GEJ), gastric adenocarcinoma, neuroendocrine tumors, gastrointestinal stromal tumors (GIST), gastric cardia adenocarcinoma, renal cancer, biliary cancer, cholangiocarcinoma, pancreatic cancer, prostate cancer, adenocarcinoma, squamous cell carcinoma, non-squamous cell carcinoma, cutaneous squamous cell carcinoma (CSCC), ovarian cancer, endometrial cancer, fallopian tube cancer, cervical cancer, peritoneal cancer, stomach cancer, brain cancers, malignant glioma, glioblastoma, gliosarcoma, neuroblastoma, thyroid cancer, adrenocortical carcinoma, oral intra-epithelial neoplasia, esophageal cancer, nasal cavity and paranasal sinus squamous cell carcinoma, nasopharynx carcinoma, salivary gland cancer, liver cancer, basal cell carcinoma; and hepatocellular cancer (HCC). However, any cancer (e.g., patients with such cancer) in which TGFβ1 is overexpressed or is at least a predominant isoform, as determined by, for example biopsy, may be treated with an isoform-selective inhibitor of TGFβ1 in accordance with the present disclosure.

In cancer, TGFβ (e.g., TGFβ1) may be either growth promoting or growth inhibitory. As an example, in pancreatic cancers, SMAD4 wild type tumors may experience inhibited growth in response to TGFβ, but as the disease progresses, constitutively activated type II receptor is typically present. Additionally, there are SMAD4-null pancreatic cancers. In some embodiments, antibodies, antigen binding portions thereof, and/or compositions of the present disclosure are designed to selectively target components of TGFβ signaling pathways that function uniquely in one or more forms of cancer. Leukemias, or cancers of the blood or bone marrow that are characterized by an abnormal proliferation of white blood cells, i.e., leukocytes, can be divided into four major classifications including acute lymphoblastic leukemia (ALL), chronic lymphocytic leukemia (CLL), acute myelogenous leukemia or acute myeloid leukemia (AML) (AML with translocations between chromosome 10 and 11 [t(10, 11)], chromosome 8 and 21 [t(8;21)], chromosome 15 and 17 [t(15;17)], and inversions in chromosome 16 [inv(16)]; AML with multilineage dysplasia, which includes patients who have had a prior myelodysplastic syndrome (MDS) or myeloproliferative disease that transforms into AML; AML and myelodysplastic syndrome (MDS), therapy-related, which category includes patients who have had prior chemotherapy and/or radiation and subsequently develop AML or MDS; d) AML not otherwise categorized, which includes subtypes of AML that do not fall into the above categories; and e) acute leukemias of ambiguous lineage, which occur when the leukemic cells cannot be classified as either myeloid or lymphoid cells, or where both types of cells are present); and chronic myelogenous leukemia (CML).

In some embodiments, any one of the above referenced TGFβ1-positive cancer may also be TGFβ3-positive. In some embodiments, tumors that are both TGFβ1-positive and TGFβ3-positive may be TGFβ1/TGFβ3 co-dominant. In some embodiments, such cancer is carcinoma comprising a solid tumor. In some embodiments, such tumors are breast carcinoma. In some embodiments, the breast carcinoma may be of triple-negative genotype (triple-negative breast cancer). In some embodiments, subjects with TGFβ1-positive cancer have elevated levels of MDSCs. For example, such tumors may comprise MDSCs recruited to the tumor site resulting in an increased number of MDSC infiltrates. In some embodiments, elevated levels of MDSCs may be detected in the blood (i.e., circulating MDSCs). In some embodiments, subjects with breast cancer show elevated levels of C-Reactive Protein (CRP), an inflammatory marker associated with recurrence and poor prognosis. In some embodiments, subjects with breast cancer show elevated levels of IL-6.

The TGFβ inhibitors of the disclosure may be used to treat patients suffering from chronic myeloid leukemia, which is a stem cell disease, in which the BCR/ABL oncoprotein is considered essential for abnormal growth and accumulation of neoplastic cells. Imatinib is an approved therapy to treat this condition; however, a significant fraction of myeloid leukemia patients show Imatinib-resistance. TGFβ inhibition achieved by the inhibitor such as those described herein may potentiate repopulation/expansion to counter BCR/ABL-driven abnormal growth and accumulation of neoplastic cells, thereby providing clinical benefit.

TGFβ inhibitors such as those described herein may be used to treat multiple myeloma. Multiple myeloma is a cancer of B lymphocytes (e.g., plasma cells, plasmablasts, memory B cells) that develops and expands in the bone marrow, causing destructive bone lesions (i.e., osteolytic lesion). Typically, the disease manifests enhanced osteoclastic bone resorption, suppressed osteoblast differentiation (e.g., differentiation arrest) and impaired bone formation, characterized in part, by osteolytic lesions, osteopenia, osteoporosis, hypercalcemia, as well as plasmacytoma, thrombocytopenia, neutropenia and neuropathy. The TGFβ inhibitor therapy described herein may be effective to ameliorate one or more such clinical manifestations or symptoms in patients. The TGFβ1 inhibitor may be administered to patients who receive additional therapy or therapies to treat multiple myeloma, including those listed elsewhere herein. In some embodiments, multiple myeloma may be treated with a TGFβ inhibitor such as an isoform-specific context-independent inhibitor, e.g., Ab6, in combination with a myostatin inhibitor (such as an antibody disclosed in WO 2017/049011, e.g., apitegromab, also known as SRK-015) or an IL-6 inhibitor. In some embodiments, the TGFβ inhibitor may be used in conjunction with traditional multiple myeloma therapies, such as bortezomib, lenalidomide, carfilzomib, pomalidomide, thalidomide, doxorubicin, corticosteroids (e.g., dexamethasone and prednisone), chemotherapy (e.g., melphalan), radiation therapy (including radiotherapeutic agents), stem cell transplantation, plitidepsin, elotuzumab, Ixazomib, masitinib, and/or panobinostat.

The types of carcinomas which may be treated by the methods of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

TGFβ inhibitors such as those described herein may be suited for treating malignancies involving cells of neural crest origin. Cancers of the neural crest lineage (i.e., neural crest-derived tumors) include, but are not limited to: melanoma (cancer of melanocytes), neuroblastoma (cancer of sympathoadrenal precursors), ganglioneuroma (cancer of peripheral nervous system ganglia), medullary thyroid carcinoma (cancer of thyroid C cells), pheochromocytoma (cancer of chromaffin cells of the adrenal medulla), and MPNST (cancer of Schwann cells). In some embodiments, antibodies and methods of the disclosure may be used to treat one or more types of cancer or cancer-related conditions that may include, but are not limited to, colon cancer, renal cancer, breast cancer, malignant melanoma, urothelial carcinoma, and glioblastoma (Schlingensiepen et al., 2008. Cancer Res. 177: 137-50; Ouhtit et al., 2013. J Cancer. 4 (7): 566-572).

### Immunological Characteristics

Under normal conditions, regulatory T cells (Tregs) represent a small subset of the overall CD4-positive lymphocyte population and play key roles for maintaining immune system in homeostasis. In nearly all cancers, however, the number of Tregs is markedly increased. While Tregs play an important role in dampening immune responses in healthy individuals, an elevated number of Tregs in cancer has been associated with poor prognosis. Elevated Tregs in cancer may dampen the host's anti-cancer immunity and may contribute to tumor progression, metastasis, tumor recurrence and/or treatment resistance. For example, human ovarian cancer ascites are infiltrated with Foxp3+ GARP+ Tregs (Downs-Canner et al., Nat Commun. 2017, 8: 14649). Similarly, Tregs positively correlated with a more immunosuppressive and more aggressive phenotype in advanced hepatocellular carcinoma (Kalathil et al., Cancer Res. 2013, 73(8): 2435-44). Tregs can suppress the proliferation of effector T cells. In addition, Tregs exert contact-dependent inhibition of immune cells (e.g., naïve CD4+ T cells) through the production of TGFβ1. To combat a tumor, therefore, it is advantageous to inhibit Tregs so sufficient effector T cells can be available to exert anti-tumor effects.

Increasing lines of evidence suggest the role of macrophages in tumor/cancer progression. The present disclosure encompasses the notion that this is in part mediated by TGFβ activation, especially TGFβ1 activation, in the tumor microenvironment. Bone marrow-derived monocytes (e.g., CD11b+) are recruited to tumor sites in response to tumor-derived cytokines/chemokines (such as CCL2, CCL3 and CCL4), where monocytes undergo differentiation and polarization to acquire pro-cancer phenotype (e.g., M2-biased or M2-like macrophages, TAMs). As previously demonstrated (WO 2018/129329), monocytes isolated from human PBMCs can be induced to polarize into different subtypes of macrophages, e.g., M1 (pro-fibrotic, anti-cancer) and M2 (pro-cancer). A majority of TAMs in many tumors are M2-biased. Among the M2-like macrophages, M2c and M2d subtypes, but not M1, are found to express elevated LRRC33 on the cell surface. Moreover, macrophages can be further skewed or activated by certain cytokine exposure, such as M-CSF, resulting in a marked increase in LRRC33 expression, which coincides with TGFβ1 expression. Increased levels of circulating M-CSF (i.e., serum M-CSF concentrations) in patients with myeloproliferative disease (e.g., myelofibrosis) have also been observed. Generally, tumors with high macrophage (TAM) and/or MDSC infiltrate are associated with poor prognosis. Similarly, elevated levels of M-CSF are also indicative of poor prognosis. Thus, in some embodiments, the TGFβ inhibitors such as those encompassed herein can be used in the treatment of cancer that is characterized by elevated levels of pro-cancer macrophages and/or MDSCs. In some embodiments, the TGFβ inhibitors such as those encompassed herein can be used in the treatment of cancer that is characterized by elevated levels of MDSCs regardless of levels of other macrophages. The LRRC33-arm of the inhibitors may at least in part mediate its inhibitory effects against disease-associated immunosuppressive myeloid cells, e.g., M2-macrophages and MDSCs.

High prevalence of tumor-associated M2-like macrophages is recapitulated in murine syngeneic tumor models described herein. In MBT-2 tumors, for example, nearly 40% of CD45-positive cells isolated from an established tumor are M2 macrophages. This is reduced by half in animals treated with a combination of an isoform-selective TGFβ1 and anti-PD-1. By comparison, no significant change in the number of tumor-associated M1 macrophages is observed in the same animals. Like M2 macrophages, tumor-associated MDSCs are also elevated in established tumors (about 10-12% of CD45+ cells) and are markedly reduced (to negligible levels) by inhibiting both PD-1 and TGFβ1 in the treated animals. As disclosed herein, a majority of tumor-infiltrating M2 macrophages and MDSCs express cell-surface LRRC33 and/or LRRC33-proTGFβ1 complex. Interestingly, cell-surface expression of LRRC33 (or LRRC33-proTGFβ1 complex) appears to be highly regulated. The TGFβ inhibitors described herein, e.g., Ab6, are capable of becoming rapidly internalized in cells expressing LRRC33 and proTGFβ1, and the rate of internalization achieved with the TGFβ inhibitor is significantly higher than that with a reference antibody that recognizes cell-surface LRRC33. Similar results are obtained from primary human macrophages. These observations show that Ab6 can promote internalization upon binding to its target, LRRC33-proTGFβ1, thereby removing the LRRC33-containing complexes from the cell surface. Thus, target engagement by a TGFβ inhibitor of the present disclosure, e.g., Ab6 may induce antibody-dependent downregulation of the target protein (e.g., cell-associated proTGFβ1 complexes). At the disease loci, this may reduce the availability of activatable latent LRRC33-proTGFβ1 levels. Therefore, the TGFβ inhibitors of the disclosure may inhibit the LRRC33 arm of TGFβ1 via dual mechanisms of action: i) blocking the release of mature growth factor from the latent complex; and, ii) removing LRRC33-proTGFβ1 complexes from cell-surface via internalization. In the tumor microenvironment, the antibodies may target cell-associated latent proTGFβ1 complexes, augmenting the inhibitory effects on the target cells, such as M2 macrophages (e.g., TAMs), MDSCs, and Tregs. Phenotypically, these are immunosuppressive cells, contributing to the immunosuppressive tumor microenvironment, which is at least in part mediated by the TGFβ1 pathway. Given that many tumors are enriched with these cells, the antibodies that are capable of targeting multiple arms of TGFβ1 function, such as those described herein, should provide a particular functional advantage.

Many human cancers are known to cause elevated levels of MDSCs in patients, as compared to healthy control (reviewed, for example, in Elliott et al., (2017) “Human tumor-infiltrating myeloid cells: phenotypic and functional diversity” Frontiers in Immunology, Vol. 8, Article 86). These human cancers include but are not limited to: bladder cancer, colorectal cancer, prostate cancer, breast cancer, glioblastoma, hepatocellular carcinoma, head and neck squamous cell carcinoma, lung cancer, melanoma, NSCL, ovarian cancer, pancreatic cancer, and renal cell carcinoma. Elevated levels of MDSCs may be detected in biological samples such as peripheral blood mononuclear cell (PBMC) and tissue samples (e.g., tumor biopsy). For example, frequency of or changes in the number of MDSCs may be measured as: percent (%) of total PBMCs, percent (%) of CD14+ cells, percent (%) of CD45+ cells; percent (%) of mononuclear cells, percent (%) of total cells, percent (%) of CD11 b+ cells, percent (%) of monocytes, percent (%) of non-lymphocytic MNCs, percent (%) of KLA-DR cells, using suitable cell surface markers (phenotype).

On the other hand, macrophage infiltration into a tumor may also signify effectiveness of a therapy. As exemplified herein, tumors effectively penetrated by effector T cells (e.g., CD8+ T cells) following the treatment with a combination of a checkpoint inhibitor and a context-independent TGFβ1 inhibitor. Intratumoral effector T cells may lead to recruitment of phagocytic monocytes/macrophages that clean up cell debris.

It was observed that the combination of anti-PD-1 and a TGFβ inhibitor resulted in robust CD8 T cell influx/expansion throughout the tumor, as compared to anti-PD-1 treatment alone. Correspondingly, robust increase in CD8 effector genes may be achieved by the combination treatment. Thus, the TGFβ1 inhibitors of the present disclosure may be used to promote effector T-cell infiltration into tumors.

In addition, extensive infiltration/expansion of the tumor by F4/80-positive macrophages is observed. This may be indicative of M1 (anti-tumor) macrophages clearing cancer cell debris generated by cytotoxic cells and is presumably a direct consequence of TGFβ1 inhibition. As described in further detail in the Examples herein, these tumor-infiltrating macrophages are identified predominantly as non-M2 macrophages for their lack of CD163 expression, indicating that circulating monocytes are recruited to the tumor site upon checkpoint inhibitor and TGFβ1 inhibitor treatment and differentiate into M1 macrophages, and this observation is accompanied by a marked influx of CD8+ T cells into the tumor site. Thus, the TGFβ1 inhibitors of the present disclosure may be used to increase non-M2 macrophages associated with tumor.

Recently, checkpoint blockade therapy (CBT) has become a standard of care for treating a number of cancer types. Despite the profound advances in cancer immunotherapy, primary resistance to CBT remains a major unmet need for patients; a majority of patients' cancers still fail to respond to PD-(L)1 inhibition. Retrospective analysis of urothelial cancer and melanoma tumors has recently implicated TGFβ activation as a potential driver of primary resistance, very likely via multiple mechanisms including exclusion of cytotoxic T cells from the tumor as well as their expansion within the tumor microenvironment (immune exclusion). These observations and subsequent preclinical validation have pointed to TGFβ pathway inhibition as a promising avenue for overcoming primary resistance to CBT. However, therapeutic targeting of the TGFβ pathway has been hindered by dose-limiting preclinical cardiotoxicities, most likely due to inhibition of signaling from one or more TGFβ isoforms.

Many tumors lack of primary response to CBT. In this scenario, CD8+ T cells are commonly excluded from the tumor parenchyma, suggesting that tumors may co-opt the immunomodulatory functions of TGFβ signaling to generate an immunosuppressive microenvironment. These insights from retrospective clinical tumor sample analyses provided the rationale for investigating the role of TGFβ signaling in primary resistance to CBT.

With respect to TGFβ and responses to CBT, herein we observe the prevalent expression of TGFβ1 in many human tumors, suggesting that this family member may be the key driver of this pathway's contribution to primary resistance.

Increasing evidence suggests that TGFβ may be a primary player in creating and/or maintaining immunosuppression in disease tissues, including the immune-excluded tumor environment. Therefore, TGFβ inhibition may unblock the immunosuppression and enable effector T cells (particularly cytotoxic CD8+ T cells) to access and kill target cancer cells. In addition to tumor infiltration, TGFβ inhibition may also promote CD8+ T cell expansion. Such expansion may occur in the lymph nodes and/or in the tumor (intratumorally). While the exact mechanism underlining this process has yet to be elucidated, it is contemplated that immunosuppression is at least in part mediated by immune cell-associated TGFβ1 activation involving regulatory T cells and activated macrophages. It has been reported that TGFβ directly promotes Foxp3 expression in CD4+ T cells, thereby converting them into a regulatory (immunosuppressive) phenotype (i.e., Treg). Moreover, Tregs suppress effector T cell proliferation, thereby reducing immune responses. This process is shown to be TGFβ1-dependent and likely involves GARP-associated TGFβ1 signaling. Observations in both humans and animal models have indicated that an increase in Tregs in TME is associated with poor prognosis in multiple types of cancer. In addition, Applicant has previously shown that M2-polarized macrophages exposed to tumor-derived factors such as M-CSF dramatically upregulate cell-surface expression of LRRC33, which is a presenting molecule for TGFβ1 (see, for example: PCT/US2018/031759). These so-called tumor-associated macrophages (or TAMs) are thought to contribute to the observed TGFβ1-dependent immunosuppression in TMEs and promote tumor growth.

A number of solid tumors are characterized by having tumor stroma enriched with myofibroblasts or myofibroblast-like cells. These cells produce collagenous matrix that surrounds or encases the tumor (such as desmoplasia), which at least in part may be caused by overactive TGFβ1 signaling. It is contemplated that the TGFβ1 activation is mediated via ECM-associated presenting molecules, e.g., LTBP1 and LTBP3 in the tumor stroma.

Selective inhibition of TGFβ activation, such as TGFβ1 inhibition, may be sufficient to overcome primary resistance to CBT. By targeting the prodomain of latent TGFβ1, an isoform-selective inhibitor of TGFβ1 may achieve isoform specificity and inhibit latent TGFβ1 activation.

Selective inhibition of the TGFβ pathway, such as the TGFβ1 pathway, may result in significantly improved preclinical safety versus broad inhibition of all isoform activity. Pleiotropic effects associated with broad TGFβ pathway inhibition have hindered therapeutic targeting of the TGFβ pathway. Most experimental therapeutics to date (e.g., galunisertib, LY3200882, fresolimumab) lack selectivity for a single TGFβ isoform, potentially contributing to the dose-limiting toxicities observed in nonclinical and clinical studies. Genetic data from knockout mice and human loss-of-function mutations in the TGFβ2 or TGFβ3 genes suggest that the cardiac toxicities observed with nonspecific TGFβ inhibitors may be due to inhibition of TGFβ2 or TGFβ3. The present disclosure teaches that selective inhibition of TGFβ1 activation with such an antibody has an improved safety profile and is sufficient to elicit robust antitumor responses when combined with PD-1 blockade, enabling the evaluation of the TGFβ1 inhibitor efficacy at clinically tractable dose levels.

The preclinical studies and results presented herein demonstrate that combination treatment with a TGFβ1 inhibitor (e.g., Ab6) and a checkpoint inhibitor may have profound effects on the intratumoral immune contexture (e.g., increased levels of tumor-associated CD8+ T cells). These may include an unexpected enrichment of Treg cells by the combination treatment with anti-PD-1/TGFβ1 inhibitor.

In addition to the expected and observed impact on the disposition of cytotoxic T cells within tumors, the TGFβ inhibitor/anti-PD-1 combination treatment may also beneficially impact the immunosuppressive myeloid compartment. Therefore, a therapeutic strategy that includes targeting of these important immunosuppressive cell types may have a greater effect than targeting a single immunosuppressive cell type (i.e., only Treg cells) in the tumor microenvironment. Thus, the TGFβ1 inhibitors of the present disclosure may be used to reduce tumor-associated immunosuppressive cells, such as M2 macrophages and MDSCs.

The preclinical studies and results presented herein demonstrate that highly specific inhibition of TGFβ1 activation may enable the host immune system to overcome a key mechanism of primary resistance to checkpoint blockade therapy, while avoiding the previously recognized toxicities of broader TGFβ inhibition that have been a key limitation for clinical application.

Accordingly, TGFβ inhibitors such as selective TGFβ1 inhibitors may be used to counter primary resistance to CBT, thereby rendering the tumor/cancer more susceptible to the CBT. Such effects may be applicable to treating a wide spectrum of malignancy types, where the cancer/tumor is TGFβ1-positive. In some embodiments, such tumor/cancer may further express additional isoform, such as TGFβ3. Non-limiting examples of the latter may include certain types of carcinoma, such as breast cancer.

Accordingly, the disclosure provides, in some embodiments, selection criteria for identifying or selecting a patient or patient populations/sub-populations for which the TGFβ1 inhibitors are likely to achieve clinical benefit. In some embodiments, suitable phenotypes of human tumors include: i) a subset(s) are shown to be responsive to CBT (e.g., PD-(L)1 axis blockade); ii) evidence of immune exclusion; and/or, iii) evidence of TGFB1 expression and/or TGFβ signaling. Various cancer types fit the profile, including, for example, melanoma and bladder cancer.

As mentioned above, TGFβ inhibitors such as those described herein may be used in the treatment of melanoma. The types of melanoma that may be treated with such inhibitors include, but are not limited to, Lentigo maligna, Lentigo maligna melanoma, Superficial spreading melanoma, Acral lentiginous melanoma, Mucosal melanoma, Nodular melanoma, Polypoid melanoma, and Desmoplastic melanoma. In some embodiments, the melanoma is a metastatic melanoma. In some embodiments, the melanoma is a cutaneous melanoma.

More recently, immune checkpoint inhibitors have been used to effectively treat advanced melanoma patients. In particular, anti-programmed death (PD)-1 antibodies (e.g., nivolumab, budigalimab and pembrolizumab) have now become the standard of care for certain types of cancer such as advanced melanoma, which have demonstrated significant activity and durable response with a manageable toxicity profile. However, effective clinical application of PD-1 antagonists is encumbered by a high rate of innate resistance (~60-70%) (see Hugo et al., (2016) Cell 165: 35-44), illustrating that ongoing challenges continue to include the questions of patient selection and predictors of response and resistance as well as optimizing combination strategies (Perrot et al., (2013) Ann Dermatol 25(2): 135-144). Moreover, studies have suggested that approximately 25% of melanoma patients who initially responded to an anti-PD-1 therapy eventually developed acquired resistance (Ribas et al., (2016) JAMA 315: 1600-9).

The number of tumor-infiltrating CD8+ T cells expressing PD-1 and/or CTLA-4 appears to be a key indicator of success with checkpoint inhibition, and both PD-1 and CTLA-4 blockade may increase the infiltrating T cells. In patients with higher presence of tumor-associated macrophages, however, anti-cancer effects of the CD8 cells may be suppressed.

It is contemplated that LRRC33-expressing cells, such as myeloid cells, including myeloid precursors, MDSCs and TAMs, may create or support an immunosuppressive environment (such as TME and myelofibrotic bone marrow) by inhibiting T cells (e.g., T cell depletion), such as CD4 and/or CD8 T cells, which may at least in part underline the observed anti-PD-1 resistance in certain patient populations. Indeed, evidence suggests that resistance to anti-PD-1 monotherapy was marked by failure to accumulate CD8+ cytotoxic T cells and reduced Teff/Treg ratio. Notably, the present inventors have recognized that there is a bifurcation among certain cancer patients, such as a melanoma patient population, with respect to LRRC33 expression levels: one group exhibits high LRRC33 expression (LRRC33^{high}), while the other group exhibits relatively low LRRC33 expression (LRRC33^{low}). Thus, the disclosure includes the notion that the LRRC33^{high} patient population may represent those who are poorly responsive to or resistant to immune checkpoint inhibitor therapy. Accordingly, agents that inhibit LRRC33, such as those described herein, may be particularly beneficial for the treatment of cancer, such as melanoma, lymphoma, and myeloproliferative disorders, that is resistant to checkpoint inhibitor therapy (e.g., anti-PD-1).

In some embodiments, cancer/tumor is intrinsically resistant to or unresponsive to an immune checkpoint inhibitor (e.g., primary resistance). Without intending to be bound by particular theory, the inventors of the present disclosure contemplate that this may be at least partly due to upregulation of TGFβ1 signaling pathways, which may create an immunosuppressive microenvironment where checkpoint inhibitors fail to exert their effects. TGFβ1 inhibition may render such cancer more responsive to checkpoint inhibitor therapy. Non-limiting examples of cancer types which may benefit from a combination of an immune checkpoint inhibitor and a TGFβ1 inhibitor include: myelofibrosis, melanoma, renal cell carcinoma, bladder cancer, colon cancer, hematologic malignancies, non-small cell carcinoma, non-small cell lung cancer/carcinoma (NSCLC), lymphoma (classical Hodgkin's and non-Hodgkin's), head and neck cancer, urothelial cancer e.g., metastatic urothelial carcinoma), cancer with high microsatellite instability, cancer with mismatch repair deficiency, gastric cancer, renal cancer, and hepatocellular cancer. However, any cancer (e.g., patients with such cancer) in which TGFβ1 is overexpressed, is co-expressed with TGFβ3, or is the dominant isoform over TGFβ2/3, as determined by, for example biopsy, may be treated with a TGFβ inhibitor in accordance with the present disclosure.

In some embodiments, a cancer/tumor becomes resistant over time. This phenomenon is referred to as acquired resistance. Like primary resistance, in some embodiments, acquired resistance is at least in part mediated by TGFβ1-dependent pathways. TGFβ inhibitors described herein may be effective in restoring anti-cancer immunity in these cases. The TGFβ inhibitors of the present disclosure may be used to reduce recurrence of tumor. The TGFβ inhibitors of the present disclosure may be used to enhance durability of cancer therapy such as CBT. The term “durability” used in the context of therapies refers to the time between clinical effects (e.g., tumor control) and tumor re-growth (e.g., recurrence). Presumably, durability and recurrence may correlate with secondary or acquired resistance, where the therapy to which the patient initially responded stops working. Thus, the TGFβ inhibitors of the present disclosure may be used to increase the duration of time the cancer therapy remains effective. The TGFβ inhibitors of the present disclosure may be used to reduce the probability of developing acquired resistance among the responders of the therapy. The TGFβ inhibitors of the present disclosure may be used to enhance progression-free survival in patients. In some embodiments, the TGFβ inhibitors described herein may be used to improve disease-free survival time in patients. In some embodiments, the TGFβ inhibitors of the present disclosure may be effective for improving patient-reported outcomes, reduced complications, faster time to treatment completion, more durable treatment, longer time between retreatment, etc. In some embodiments, the TGFβ inhibitors of the present disclosure may be used to improve overall survival in patients.

In some embodiments, the TGFβ inhibitors of the present disclosure may be used to improve rates or ratios of complete verses partial responses among the responders of a cancer therapy. Typically, even in cancer types where response rates to a cancer therapy (such as CBT) are relatively high (e.g., ≥35%), CR rates are quite low. The TGFβ inhibitors of the present disclosure are therefore used to increase the fraction of complete responders within the responder population.

In addition, the TGFβ inhibitor may be also effective to enhance or augment the degree of partial response among partial responders.

In some embodiments, clinical endpoints for the TGFβ inhibitors described herein include those described in the 2018 Food and Drug Administration Guidelines for Clinical Trial Endpoints for the Approval of Cancer Drugs and Biologics, the content of which is incorporated herein in its entirety.

In some embodiments, combination therapy comprising an immune checkpoint inhibitor and an LRRC33 inhibitor (such as those described herein) may be used with the methods disclosed herein and may be effective to treat such cancer. In addition, high LRRC33-positive cell infiltrate in tumors, or otherwise sites/tissues with abnormal cell proliferation, may serve as a biomarker for host immunosuppression and immune checkpoint resistance. Similarly, effector T cells may be precluded from the immunosuppressive niche which limits the body's ability to combat cancer.

As demonstrated in the Example section below, Tregs that express GARP-presented TGFβ1 suppress effector T cell proliferation. Together, TGFβ1 is likely a key driver in the generation and maintenance of an immune inhibitory disease microenvironment (such as TME), and multiple TGFβ1 presentation contexts are relevant for tumors. In some embodiments, the combination therapy may achieve more favorable Teff/Treg ratios.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating cancer in a subject in need thereof, said method comprising administering the antibody, or antigen binding portion thereof, to the subject such that the cancer is treated. In certain embodiments, the cancer is colon cancer. In certain embodiments, the cancer is melanoma. In certain embodiments, the cancer is bladder cancer. In certain embodiments, the cancer is head and neck cancer. In certain embodiments, the cancer is lung cancer.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating solid tumors. In some embodiments, solid tumors may be desmoplastic tumors, which are typically dense and hard for therapeutic molecules to penetrate. By targeting the ECM component of such tumors, such antibodies may “loosen” the dense tumor tissue to disintegrate, facilitating therapeutic access to exert its anti-cancer effects. Thus, additional therapeutics, such as any known anti-tumor drugs, may be used in combination. In preferred embodiments, the isoform-selective activation inhibitor of TGFβ1 is Ab46, Ab50, a derivative thereof, or an engineered molecule comprising an antigen-binding fragment thereof.

Additionally or alternatively, isoform-specific, context-independent antibodies for fragments thereof that are capable of inhibiting TGFβ1 activation, such as those disclosed herein, may be used in conjunction with the chimeric antigen receptor T-cell (“CAR-T”) technology as cell-based immunotherapy, such as cancer immunotherapy for combatting cancer.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for inhibiting or decreasing solid tumor growth in a subject having a solid tumor, said method comprising administering the antibody, or antigen binding portion thereof, to the subject such that the solid tumor growth is inhibited or decreased. In certain embodiments, the solid tumor is a colon carcinoma tumor. In some embodiments, the antibodies, or antigen binding portions thereof useful for treating a cancer is an isoform-specific, context-independent inhibitor of TGFβ1 activation. In some embodiments, such antibodies target a GARP-TGFβ1 complex, a LTBP1-TGFβ1 complex, a LTBP3-TGFβ1 complex, and a LRRC33-TGFβ1 complex. In some embodiments, such antibodies target a GARP-TGFβ1 complex, a LTBP1-TGFβ1 complex, and a LTBP3-TGFβ1 complex. In some embodiments, such antibodies target a LTBP1-TGFβ1 complex, a LTBP3-TGFβ1 complex, and a LRRC33-TGFβ1 complex. In some embodiments, such antibodies target a GARP-TGFβ1 complex and a LRRC33-TGFβ1 complex.

The disclosure includes the use of TGFβ inhibitors, such as context-independent, isoform-specific inhibitors of TGFβ1, in the treatment of cancer comprising a solid tumor in a subject, e.g., a solid tumor that is a CD8+ T cell-infiltrated tumor. In some embodiments, such TGFβ inhibitors may inhibit the activation of TGFβ1. In some embodiments, such TGFβ inhibitors comprise an antibody or antigen-binding portion thereof that binds a proTGFβ1 complex. The binding can occur when the complex is associated with any one of the presenting molecules, e.g., LTBP1, LTBP3, GARP or LRRC33, thereby inhibiting release of mature TGFβ1 growth factor from the complex. In some embodiments, the solid tumor is characterized by having stroma enriched with CD8+ T cells making direct contact with CAFs and collagen fibers. Such a tumor may create an immuno-suppressive environment that prevents anti-tumor immune cells (e.g., effector T cells) from effectively infiltrating the tumor or expanding within the tumor, limiting the body's ability to fight cancer. Instead, such cells may accumulate within or near the tumor stroma. These features may render such tumors poorly responsive to an immune checkpoint inhibitor therapy. As discussed in more detail below, TGFβ1 inhibitors disclosed herein may unblock the suppression so as to allow effector cells to reach and kill cancer cells, for example, used in conjunction with an immune checkpoint inhibitor. In preferred embodiments, the isoform-selective activation inhibitor of TGFβ1 is Ab46, Ab50, a derivative thereof, or an engineered molecule comprising an antigen-binding fragment thereof.

TGFβ, especially TGFβ1, is contemplated to play multifaceted roles in a tumor microenvironment, including tumor growth, host immune suppression, malignant cell proliferation, vascularity, angiogenesis, migration, invasion, metastasis, and chemo-resistance. Each “context” of TGFβ1 presentation in the environment may therefore participate in the regulation (or dysregulation) of disease progression. For example, the GARP axis is particularly important in Treg response that regulates effector T cell response for mediating host immune response to combat cancer cells. The LTBP1/3 axis may regulate the ECM, including the stroma, where cancer-associated fibroblasts (CAFs) play a role in the pathogenesis and progression of cancer. The LRRC33 axis may play a crucial role in recruitment of circulating monocytes to the tumor microenvironment, subsequent differentiation into tumor-associated macrophages (TAMs), infiltration into the tumor tissue and exacerbation of the disease.

In some embodiments, TGFβ1-expressing cells infiltrate the tumor, creating or contributing to an immunosuppressive local environment. The degree by which such infiltration is observed may correlate with worse prognosis. In some embodiments, higher infiltration is indicative of poorer treatment response to another cancer therapy, such as immune checkpoint inhibitors. In some embodiments, TGFβ1-expressing cells in the tumor microenvironment comprise immunosuppressive immune cells such as Tregs and/or myeloid cells. In some embodiments, the myeloid cells include, but are not limited to, macrophages, monocytes (tissue resident or bone marrow-derived), and MDSCs.

In some embodiments, LRRC33-expressing cells in the TME are myeloid-derived suppressor cells (MDSCs). MDSC infiltration (e.g., solid tumor infiltrate) may underline at least one mechanism of immune escape, by creating an immunosuppressive niche from which host's anti-tumor immune cells become excluded. Evidence suggest that MDSCs are mobilized by inflammation-associated signals, such as tumor-associated inflammatory factors, Opon mobilization, MDSCs can influence immunosuppressive effects by impairing disease-combating cells, such as CD8+ T cells and NK cells. In addition, MDSCs may induce differentiation of Tregs by secreting TGFβ and IL-10, further adding to the immunosuppressive effects. Thus, TGFβ inhibitor such as those described herein may be administered to patients with immune evasion (e.g., compromised immune surveillance) to restore or boost the body's ability to fight the disease (such as a cancer or tumor). As described in more detail herein, this may further enhance (e.g., restore or potentiate) the body's responsiveness or sensitivity to another therapy, such as cancer therapy.

In some embodiments, elevated frequencies (e.g., number) of circulating MDSCs in patients are predictive of poor responsiveness to checkpoint blockade therapies, such as PD-1 antagonists and PD-L1 antagonists. For example, biomarker studies showed that circulating pre-treatment HLA-DR^{lo}/CD14+/CD11 b+ myeloid-derived suppressor cells (MDSC) were associated with progression and worse OS (p=0.0001 and 0.0009). In addition, resistance to PD-1 checkpoint blockade in inflamed head and neck carcinoma (HNC) associates with expression of GM-CSF and Myeloid Derived Suppressor Cell (MDSC) markers. This observation suggested that strategies to deplete MDSCs, such as chemotherapy, should be considered in combination (e.g., administered concurrently (e.g., simultaneously), separately, or sequentially) with anti-PD-1. LRRC33 or LRRC33-TGFβcomplexes represent a novel target for cancer immunotherapy due to selective expression on immunosuppressive myeloid cells. Therefore, without intending to be bound by particular theory, targeting this complex may enhance the effectiveness of standard-of-care checkpoint inhibitor therapies in the patient population.

The invention therefore provides the use of an isoform-specific, TGFβ1 inhibitor described herein for the treatment of cancer that comprises a solid tumor. Such treatment comprises administration of the isoform-specific, TGFβ1 inhibitor to a subject diagnosed with cancer that includes at least one localized tumor (solid tumor) in an amount effective to treat the cancer. In preferred embodiments, the isoform-selective activation inhibitor of TGFβ1 is Ab46, Ab50, a derivative thereof, or an engineered molecule comprising an antigen-binding fragment thereof.

The disclosure therefore provides the use of TGFβ inhibitors, such as the isoform-specific TGFβ1 inhibitor described herein, for the treatment of cancer that comprises a solid tumor. Such treatment comprises administration of a TGFβ inhibitor encompassed by the disclosure, e.g., Ab6, to a subject diagnosed with cancer that includes at least one localized tumor (solid tumor) in an amount effective to treat the cancer. Preferably, the subject is further treated with a cancer therapy, such as CBT, chemotherapy, and/or radiation therapy (such as a radiotherapeutic agent). In some embodiments, the TGFβ inhibitor increases the rate/fraction of a primary responder patient population to the cancer therapy. In some embodiments, the TGFβ inhibitor increases the degree of responsiveness of primary responders to the cancer therapy. In some embodiments, the TGF1 inhibitor increases the ratio of complete responders to partial responders to the cancer therapy. In some embodiments, the TGFβ inhibitor increases the durability of the cancer therapy such that the duration before recurrence and/or before the cancer therapy becomes ineffective is prolonged. In some embodiments, the TGFβ inhibitor reduces occurrences or probability of acquired resistance to the cancer therapy among primary responders.

In some embodiments, cancer progression (e.g., tumor proliferation/growth, invasion, angiogenesis and metastasis) may be at least in part driven by tumor-stroma interaction. In particular, CAFs may contribute to this process by secretion of various cytokines and growth factors and ECM remodeling. Factors involved in the process include but are not limited to stromal-cell-derived factor 1 (SCD-1), MMP2, MMP9, MMP3, MMP-13, TNF-α, TGFβ1, VEGF, IL-6, M-CSF. In addition, CAFs may recruit TAMs by secreting factors such as CCL2/MCP-1 and SDF-1/CXCL12 to a tumor site; subsequently, a pro-TAM niche (e.g., hyaluronan-enriched stromal areas) is created where TAMs preferentially attach. Since TGFβ1 has been suggested to promote activation of normal fibroblasts into myofibroblast-like CAFs, administration of an isoform-specific, context-independent TGFβ1 inhibitor such as those described herein may be effective to counter cancer-promoting activities of CAFs. Data presented herein suggest that an isoform-specific context-independent antibody that blocks activation of TGFβ1 can inhibit UUO-induced upregulation of maker genes such as CCL2/MCP-1, α-SMA. FN1 and Col1, which are also implicated in many cancers.

In certain embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, are administered to a subject having cancer or a tumor, either alone or in combination with an additional agent, e.g., an anti-PD-1 antibody (e.g., an anti-PD-1 antagonist). Other combination therapies which are included in the disclosure are the administration of an antibody, or antigen binding portion thereof, described herein, with radiation (radiation therapy, including radiotherapeutic agents), or a chemotherapeutic agent (chemotherapy). Exemplary additional agents to use with an anti-TGFβ inhibitor include, but are not limited to, a PD-1 antagonist (e.g., a PD-1 antibody), a PDL1 antagonist (e.g., a PDL1 antibody), a PD-L1 or PDL2 fusion protein, a CTLA4 antagonist (e.g., a CTLA4 antibody), a GITR agonist e.g., a GITR antibody), an anti-ICOS antibody, an anti-ICOSL antibody, an anti-B7H3 antibody, an anti-B7H4 antibody, an anti-TIM3 antibody, an anti-LAG3 antibody, an anti-OX40 antibody (OX40 agonist), an anti-CD27 antibody, an anti-CD70 antibody, an anti-CD47 antibody, an anti-41BB antibody, an anti-PD-1 antibody, an anti-CD20 antibody, an anti-CD3 antibody, an anti-CD3/anti-CD20 bispecific or multispecific antibody, an anti-HER2 antibody, an anti-CD79b antibody, an anti-CD47 antibody, an antibody that binds T cell immunoglobulin and ITIM domain protein (TIGIT), an anti-ST2 antibody, an anti-beta7 integrin (e.g., an anti-alpha4-beta7 integrin and/or alphaE beta7 integrin), a CDK inhibitor, an oncolytic virus, an indoleamine 2,3-dioxygenase (IDO) inhibitor, and/or a PARP inhibitor. Examples of useful oncolytic viruses include, adenovirus, reovirus, measles, herpes simplex, Newcastle disease virus, senecavirus, enterovirus and vaccinia. In certain embodiments, the oncolytic virus is engineered for tumor selectivity.

In some embodiments, determination or selection of therapeutic approach for combination therapy that suits particular cancer types or patient population may involve the following: a) considerations regarding cancer types for which a standard-of-care therapy is available (e.g., immunotherapy-approved indications); b) considerations regarding treatment-resistant subpopulations (e.g., immune excluded); and c) considerations regarding cancers/tumors that are or generally suspected to be “TGFβ1 pathway-active” or otherwise at least in part TGFβ1-dependent (e.g., TGFβ1 inhibition-sensitive). For example, many cancer samples show that TGFβ1 is the predominant isoform by, for instance, TCGA RNAseq. In some embodiments, DNA- and/or RNA-based assays (e.g., RNAseq or Nanostring) may be used to evaluate the level of TGFβ signaling (e.g., TGFβ1 signaling) in tumor samples. In some embodiments, over 50% (e.g., over 50%, 60%, 70%, 80% and 90%) of samples from each tumor type are positive for TGFβ1 isoform expression. In some embodiments, the cancers/tumors that are “TGFβ1 pathway-active” or otherwise at least in part TGFβ1-dependent (e.g., TGFβ1 inhibition-sensitive) contain at least one Ras mutation, such as mutations in K-ras, N-ras and/or H-ras. In some embodiments, the cancer/tumor comprises at least one K-ras mutation.

Confirmation of TGFβ1 expression in clinical samples collected from patients (such as biopsy samples) is not prerequisite to TGFβ1 inhibition therapy, where the particular condition has been generally known or suspected to involve the TGFβ pathway.

In some embodiments, a TGFβ inhibitor such as those described herein is administered in conjunction with checkpoint inhibitory therapy to patients diagnosed with cancer for which one or more checkpoint inhibitor therapies are approved or shown effective. These include, but are not limited to: bladder urothelial carcinoma (such as metastatic urothelial carcinoma), squamous cell carcinoma (such as head & neck), kidney clear cell carcinoma, kidney papillary cell carcinoma, liver hepatocellular carcinoma, lung adenocarcinoma, skin cutaneous melanoma, and stomach adenocarcinoma. In certain embodiments, such patients are poorly responsive or non-responsive to the checkpoint inhibitor therapy. In some embodiments, the poor responsiveness is due to primary resistance. In some embodiments, the cancer that is resistant to checkpoint blockade shows downregulation of TCF7 expression. In some embodiments, TCF7 downregulation in checkpoint inhibition-resistant tumor may be correlated with a low number of intratumoral CD8+ T cells. In preferred embodiments, the isoform-selective activation inhibitor of TGFβ1 is Ab46, Ab50, a derivative thereof, or an engineered molecule comprising an antigen-binding fragment thereof.

A TGFβ inhibitor such as those described herein may be used in the treatment of chemotherapy- or radiotherapy-resistant cancers. Thus, in some embodiments, a TGFβ1 inhibitor, e.g., Ab6, may be administered to patients diagnosed with cancer for which they receive or have received chemotherapy and/or radiation therapy (such as a radiotherapeutic agent). In particular, the use of the TGFβ1 inhibitor is advantageous where the cancer (patient) is resistant to such therapy. In some embodiments, such cancer comprises quiescent tumor propagating cancer cells (TPCs), in which TGFβ signaling controls their reversible entry into a growth arrested state, which protects TPCs from chemotherapy or radiation therapy (such as a radiotherapeutic agent). It is contemplated that upon pharmacological inhibition of TGFβ1, TPCs with compromised fail to enter quiescence and thus rendered susceptible to chemotherapy and/or radiation therapy (such as a radiotherapeutic agent). Such cancer includes various carcinomas, e.g., squamous cell carcinomas. See, for example, Brown et al., (2017) “TGF-β-Induced Quiescence Mediates Chemoresistance of Tumor-Propagating Cells in Squamous Cell Carcinoma.” Cell Stem Cell. 21(5):650-664.

In some embodiments, a TGFβ inhibitor such as an isoform-selective TGFβ1 inhibitor (e.g., Ab6) may be used to treat (e.g., reduce) anemia in a subject, e.g., in a cancer patient. In some embodiments, a TGFβ inhibitor such as an isoform-selective TGFβ1 inhibitor (e.g., Ab6) may be used in combination with a BMP inhibitor (e.g., a BMP6 inhibitor, e.g., a RGMc inhibitor, e.g., any of the RGMc inhibitor disclosed in WO/2020/086736, the content of which is hereby incorporated in its entirety) to treat (e.g., reduce) anemia, e.g., in the subject. In some embodiments, the anemia results from reduced or impaired red blood cell production (e.g., as a result of myelofibrosis or cancer), iron restriction (e.g., as a result of cancer or treatment-induced anemia, such as chemotherapy-induced anemia), or both. In some embodiments, the combination of a TGFβ inhibitor and a BMP inhibitor (antagonist) may be administered at a therapeutically effective amount or amounts that is/are sufficient to relieve one or more anemia-related symptom and/or complication in the subject, e.g., a cancer patient. In some embodiments, the combination of a TGFβ inhibitor and a BMP inhibitor (antagonist) may be administered at a therapeutically effective amount that is sufficient to increase or normalize red blood cell production and/or reduce iron restriction. Without wishing to be bound by theory, it is contemplated that TGFβ1 inhibitors (e.g., Ab6) may alleviate symptoms and/or complications related to anemia through their hematopoiesis-promoting effects and that BMP inhibitors (antagonists) (e.g., a BMP6 inhibitor, e.g., a RGMc inhibitor) may improve iron-deficiency anemia (e.g., chemotherapy-induced anemia). In some embodiments, the treatment for anemia further comprises administering one or more JAK inhibitor (e.g., Jak1/2 inhibitor, Jak1 inhibitor, and/or Jak2 inhibitor).

In some embodiments, the BMP inhibitor is an antagonist of the kinase associated with the BMP receptor (e.g., type I receptor and/or type II receptor).

In some embodiments, the BMP inhibitor is a “ligand trap” that binds (or sequesters) the BMP growth factor(s), including BMP6.

In some embodiments, the BMP inhibitor is an antibody that neutralizes the BMP growth factor(s), including BMP6. Examples include anti-BMP6 antibodies (e.g., WO 2016/098079, Novartis; and, KY-1070, KyMab).

In some embodiments, the BMP inhibitor is an inhibitor of a BMP6 co-receptor, such as RGMc. For example, such inhibitor may include an antibody that binds RGMa/c. (Böser et al. AAPS J. 2015 July; 17(4): 930-938). More preferably, such inhibitor is an antibody that selectively binds RGMc (see, for example, WO 2020/086736). Therapeutic Indications and/or Subjects Likely to Benefit from a Therapy Comprising a TGFβ-Inhibitor

The current disclosure encompasses methods of treating cancer and predicting or monitoring therapeutic efficacy using a TGFβ inhibitor, e.g., Ab6. In some embodiments, the identification/screening/selection of suitable indications and/or patient populations for which TGFβ inhibitors, such as those described herein, are likely to have advantageous therapeutic benefits comprise: i) whether the disease is driven by or dependent predominantly on the TGFβ1 isoform over the other isoforms in human (or at least co-dominant); ii) whether the condition (or affected tissue) is associated with an immunosuppressive phenotype (e.g., an immune-excluded tumor); and, iii) whether the disease involves both matrix-associated and cell-associated TGFβ1 function.

Differential expression of the three known TGFβ isoforms, namely, TGFβ1, TGFβ2, and TGFβ3, has been observed under normal (healthy; homeostatic) as well as disease conditions in various tissues. Nevertheless, the concept of isoform selectivity has neither been fully exploited nor robustly achieved with conventional approaches that favor pan-inhibition of TGFβ across multiple isoforms. Moreover, expression patterns of the isoforms may be differentially regulated, not only in normal (homeostatic) vs abnormal (pathologic) conditions, but also in different subpopulations of patients. Because most preclinical studies are conducted in a limited number of animal models, which may or may not recapitulate human conditions, data obtained with the use of such models may be biased, resulting in misinterpretations of data or misleading conclusions as to the translatability for purposes of developing therapeutics.

Previous analyses of human tumor samples implicated TGFβ signaling as an important contributor to primary resistance to disease progression and treatment response, including checkpoint blockade therapy (37 CBT”) for various types of malignancies. Studies reported in literature reveal that the TGFB gene expression may be particularly relevant to treatment resistance, suggesting that activity of this isoform may be driving TGFβ signaling in these diseases. Across the majority of human tumor types profiled at The Cancer Genome Atlas (TCGA), TGFB1 expression appears to be the most prevalent, suggesting that selection of preclinical models that more closely recapitulate human disease expression patterns of TGFβ isoforms may be beneficial.

Without being bound by theory, TGFβ1 and TGFβ3 are often co-dominant (co-expressed at similar levels) in certain murine syngeneic cancer models (e.g., EMT-6 and 4T1) that are widely used in preclinical studies. By contrast, numerous other cancer models (e.g., S91, B16 and MBT-2) express almost exclusively TGFβ1, similar to that observed in many human tumors, in which TGFβ1 appears to be more frequently the dominant isoform over TGFβ2/3. Furthermore, the TGFβ isoform(s) predominantly expressed under homeostatic conditions may not be the disease-associated isoform(s). For example, in normal lung tissues in healthy rats, tonic TGFβ signaling appears to be mediated mainly by TGFβ3. However, TGFβ1 appears to become markedly upregulated in disease conditions, such as lung fibrosis. Taken together, while not prerequisite, it may be beneficial to test or confirm relative expression of TGFβ isoforms in clinical samples so as to select suitable therapeutics to which the patient is likely to respond. In some embodiments, determination of relative isoform expression may be made post-treatment. In such circumstances, patients' responsiveness (e.g., clinical response/benefit) in response to TGFβ1 inhibition therapy may be correlated with relative expression levels of TGFβ isoforms. In some embodiments, overexpression of the TGFβ1 isoform shown ex post facto correlates with greater responsiveness to the treatment.

Whilst inhibition of TGFβ1 alone appears to be sufficient to overcome primary resistance to cancer immunotherapy as demonstrated in a tumor model expressing both TGFβ1 and TGFβ3 (see Examples herein), findings disclosed herein suggests that inhibition of TGFβ3 may in fact be harmful. Surprisingly, in a murine liver fibrosis model, mice treated with an isoform-selective inhibitor of TGFβ3 manifest exacerbation of fibrosis. A significant increase of collagen deposits in liver sections of these animals suggest that inhibition of TGFβ3 in fact may result in greater dysregulation of the ECM. Without being bound by theory, this suggests that TGFβ3 inhibition may promote a pro-fibrotic phenotype.

A hallmark of pro-fibrotic phenotypes is increased deposition and/or accumulation of collagens in the ECM, which is associated with increased stiffness of tissue ECMs. This has been observed during pathological progression of cancer, fibrosis and cardiovascular disease. Consistent with this, Applicant previously demonstrated the role of matrix stiffness on integrin-dependent activation of TGFβ, using primary fibroblasts grown on silicon-based substrates with defined stiffness (e.g., 5 kPa, 15 kPa or 100 kPa) (see WO 2018/129329). Matrices with greater stiffness enhanced TGFβ1 activation, and this was suppressed by isoform-specific inhibitors of TGFβ1. These observations suggest that the pharmacologic inhibition of TGFβ3 may exert opposing effects to TGFβ1 inhibition by creating a pro-tumor microenvironment, where greater stiffness of the tissue matrix may support cancer progression.

Given the common pathways involved in fibrotic phenotypes and many aspects of cancer progression such as increased invasiveness and metastasis (see, for example: Chakravarthy et al., Nat Com (2018)9:4692. “TGF-β-associated extracellular matrix genes link cancer-associated fibroblasts to immune evasion and immunotherapy failure”), pro-fibrotic effects of TGFβ3 inhibition observed in a fibrosis model may be applicable to cancer contexts.

The finding mentioned above therefore raises the possibility that TGFβ inhibitors with inhibitory potency against TGFβ3 may not only be ineffective in treating cancer but may in fact be detrimental. In some embodiments, TGFβ3 inhibition is avoided in patients suffering from a cancer type that is statistically highly metastatic. Cancer types that are typically considered highly metastatic include, but are not limited to, colorectal cancer, lung cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, and thyroid cancer. Moreover, TGFβ3 inhibition may be best avoided in patients having or are at risk of developing a fibrotic condition and/or cardiovascular disease. Such patients at risk of developing a fibrotic condition and/or cardiovascular disease include, but are not limited to, those with metabolic disorders, such as NAFLD and NASH, obesity, and type 2 diabetes. Similarly, TGFβ3 inhibition may be best avoided in patients diagnosed with or at risk of developing myelofibrosis. Those at risk of developing myelofibrosis include those with one or more genetic mutations implicated in the pathogenesis of myelofibrosis.

In addition to the possible concerns of inhibiting TGFβ3 addressed above, Takahashi et al. (Nat Metab. 2019, 1(2): 291-303) recently reported a beneficial role of TGFβ2 in regulating metabolism. The authors identified TGFβ2 as an exercise-induced adipokine, which stimulated glucose and fatty acid uptake in vitro, as well as tissue glucose uptake in vivo; which improved metabolism in obese mice; and, which reduced high fat diet-induced inflammation. Moreover, the authors observed that lactate, a metabolite released from muscle during exercise, stimulated TGFβ2 expression in human adipocytes and that a lactate-lowering agent reduced circulating TGFβ2 levels and reduced exercise-stimulated improvements in glucose tolerance. Thus, in some embodiments, a TGFβ inhibitor may be used in treating a subject that does not have inhibitory activity towards the TGFβ2 isoform, e.g., to avoid a potentially harmful impact on one or more metabolic functions of a treated subject.

More recently, a potential link between cancer and various metabolic conditions has been recognized. For example, as reviewed by Braun et al., an enhanced risk of cancer mortality is associated with metabolic syndrome among men (Braun et al. Int J Biol Sci. 2011; 7(7): 1003-1015). Similarly, the authors noted “metabolic dysregulation may play an important role in the etiology and progression of certain cancer types and worse outcome for some cancers. Obesity and diabetes, individually, have been associated with breast, endometrial, colorectal, pancreatic, hepatic and renal cancer” (Braun et al. Int J Biol Sci. 2011; 7(7): 1003-1015).

Accordingly, in various embodiments, a TGFβ inhibitor may be used in the treatment of a TGFβ-related indication (e.g., cancer) in a subject, wherein, the TGFβ inhibitor inhibits TGFβ1 but does not inhibit TGFβ2 at the therapeutically effective dose administered. In some embodiments, the subject benefits from improved metabolism after such treatment, wherein optionally, the subject has or is at risk of developing a metabolic disease, such as obesity, high fat diet-induced inflammation, and glucose dysregulation (e.g., diabetes). In some embodiments, the TGFβ-related indication is cancer, wherein optionally the cancer comprises a solid tumor, such as locally advanced cancer and metastatic cancer. n some embodiments, the TGFβ-related indication is myelofibrosis. In some embodiments, the TGFβ-related indication is an immune disorder. In some embodiments, the TGFβ-related indication is fibrosis.

In some embodiments, the TGFβ inhibitor is TGFβ1-selective (e.g., it does not inhibit TGFβ2 and/or TGFβ3 signaling at a therapeutically effective dose). In certain embodiments, a TGFβ1-selective inhibitor is selected for use in treating a cancer patient. In some embodiments, such a treatment: i) avoids TGFβ3 inhibition to reduce the risk of exacerbating ECM dysregulation (which may contribute to tumor growth and invasiveness) and ii) avoids TGFβ2 inhibition to reduce the risk of increasing metabolic burden in the patients. Related methods for selecting a TGFβ inhibitor for therapeutic use are also encompassed herein.

The disclosure includes methods for selecting a TGFβ inhibitor for use in the treatment of cancer, wherein the TGFβ inhibitor has no or little inhibitory potency against TGFβ3 (e.g., the TGFβ inhibitor does not target TGFβ3). In certain embodiments, the TGFβ inhibitor is a TGFβ1-selective inhibitor (e.g., antibodies or antigen binding fragments that do not inhibit TGFβ2 and/or TGFβ3 signaling at therapeutically effective doses). It is contemplated that this selection strategy may reduce the risk of exacerbating ECM dysregulation in cancer patients and still provide benefits of TGFβ1 inhibition to treat cancer. In some embodiments, the cancer patients are also treated with a cancer therapy, such as immune checkpoint inhibitors. In some embodiments, the cancer patient is at risk of developing a metabolic disease, such as fatty liver, obesity, high fat diet-induced inflammation, and glucose or insulin dysregulation (e.g., diabetes).

The present disclosure also includes related methods for selecting and/or treating suitable patient populations who may be candidates for receiving a TGFβ inhibitor capable of inhibiting TGFβ3. Such methods include use of a TGFβ inhibitor capable of inhibiting TGFβ3 for the treatment of cancer in subjects who are not diagnosed with a fibrotic disorder (such as organ fibrosis), who are not diagnosed with myelofibrosis, who are not diagnosed with a cardiovascular disease and/or those who are not at risk of developing such conditions. Similarly, such methods include use of a TGFβ inhibitor capable of inhibiting TGFβ3 for the treatment of cancer in subjects, wherein the cancer is not considered to be highly metastatic. The TGFβ inhibitor capable of inhibiting TGFβ3 may include pan-inhibitors of TGFβ (such as low molecular weight antagonists of TGFβ receptors, e.g., ALK5 inhibitors, and neutralizing antibodies that bind TGFβ1/2/3), isoform-non-selective inhibitors such as antibodies that bind TGFβ1/3 and engineered fusion proteins capable of binding TGFβ1/3, e.g., ligand traps, and integrin inhibitors (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

The surprising notion that TGFβ3 inhibition may in fact be disease-promoting suggests that patients who have been previously treated with or currently undergoing treatment with a TGFβ inhibitor with inhibitory activity towards TGFβ3 may benefit from additional treatment with a TGFβ1-selective inhibitor to counter the possible pro-fibrotic effects of the TGFβ3 inhibitor. Accordingly, the disclosure includes a TGFβ1-selective inhibitor for use in the treatment of cancer in a subject, wherein the subject has been treated with a TGFβ inhibitor that inhibits TGFβ3 in conjunction with a checkpoint inhibitor, comprising the step of: administering to the subject a TGFβ1-selective inhibitor, wherein optionally the cancer is a metastatic cancer, a desmoplastic tumor, myelofibrosis, and/or, wherein the subject has a fibrotic disorder or is at risk of developing a fibrotic disorder and/or cardiovascular disease, wherein optionally the subject at risk of developing a fibrotic disorder or cardiovascular disease suffers from a metabolic condition, wherein optionally the metabolic condition is NAFLD, NASH, obesity or diabetes.

As described herein, the isoform-selective TGFβ1 inhibitors are particularly advantageous for the treatment of diseases in which the TGFβ1 isoform is predominantly expressed relative to the other isoforms (e.g., referred to as TGFβ1-dominant). Overall TGFβ1 expression (TGFB1) is significantly higher in most of these human tumors/cancers than the other two isoforms across many tumor/cancer types, suggesting that TGFβ1-selective inhibition may be beneficial in these disease types. Taken together, these lines of evidence support the notion that selective inhibition of TGFβ1 activity may overcome primary resistance to CBT. Generation of highly selective TGFβ1 inhibitors will also enable evaluation of whether such an approach will address key safety issues observed with pan-TGFβ inhibition, which will be important for assessment of their therapeutic utility.

It was previously considered that TGFβ1 inhibitors may not be efficacious, particularly in cancer types in which TGFβ1 is co-dominant with another isoform or in which TGFβ2 and/or TGFβ3 expression is significantly greater than TGFβ1. However more recently, the inventors of the present application have made an unexpected finding that TGFβ inhibitors, e.g., TGFβ1 inhibitors, such as a TGFβ1-selective inhibitor (e.g., Ab6), used in conjunction with a checkpoint inhibitor (e.g., anti-PD-1 antibody), is capable of causing significant tumor regression in the EMT-6 model, which is known to express both TGFβ1 and TGFβ3 at similar levels. The co-dominance has been confirmed by both RNA measurements and ELISA assays. This observation was surprising because it had been previously hypothesized that in order to achieve material efficacy in tumors co-expressing TGFβ1 and TGFβ3 in a checkpoint blockade context, both of the co-dominant isoforms would have to be specifically inhibited. Accordingly, methods of treatment disclosed herein include the use of TGFβ1 inhibitor for promoting tumor regression, where the tumor is TGFβ1+/TGFβ3+. Such tumor may include, for example, cancers of epithelial origin, i.e., carcinoma (e.g., basal cell carcinoma, squamous cell carcinoma, renal cell carcinoma, ductal carcinoma in situ (DCIS), invasive ductal carcinoma, and adenocarcinoma). In some embodiments, TGFβ1 is predominantly the disease-associated isoform, whilst TGFβ3 supports homeostatic function in the tissue, such as epithelia.

Aberrant activity of the TGFβ signaling pathway has been reported to impact gene expressions involved in both fibrotic and cancer processes. For instance, dysregulation of the TGFβ1 signal transduction pathway has been observed to alter genes such as SNAI1, MMP2, MMP9, and TIMP1, all of which are important for cellular processes like adhesion and extracellular matrix remodeling and have been implicated in fibrosis and the epithelial mesenchymal transition (EMT) process in cancer. Accordingly, in some embodiments, the methods of treatment herein, e.g., of fibrosis-related cancer indications, comprise the administration of a TGFβ inhibitor that does not inhibit TGFβ3, e.g., using a TGFβ1-selective antibody, e.g., Ab6. Certain tumors, such as various carcinomas, may be characterized as low mutational burden tumors (MBTs). Such tumors are often poorly immunogenic and fail to elicit sufficient T cell response. Cancer therapies that include chemotherapy, radiation therapy (such as a radiotherapeutic agent), cancer vaccines and/or oncolytic virus, may be helpful to elicit T cell immunity in such tumors. Therefore, TGFβ1 inhibition therapy detailed herein can be used in conjunction with one or more of these cancer therapies to increase anti-tumor effects. Essentially, such combination therapy is aimed at converting “cold” tumors (e.g., poorly immunogenic tumors) into “hot” tumors by promoting neo-antigens and facilitating effector cells to attack the tumor. Examples of such tumors include breast cancer, ovarian cancer, and pancreatic cancer, e.g., pancreatic ductal adenocarcinoma (PDAC).

The notion of sensitizing tumors to genotoxic agents such as chemotherapy and radiation therapy discussed above is supported by a recent report by Liu et al. (Sci Transl Med 13, eabc4465 (2021) “Loss of TGFβ signaling increases alternative end-joining DNA repair that sensitizes to genotoxic therapies across cancer types”. See below). In some embodiments, such cancer types show faulty DNA repair function necessary for maintenance of genomic integrity. The faulty DNA repair function may be associated with one or more mutations in, for example, ATM (ataxia telangiectasia mutated), BRCA1 (breast cancer 1 gene), and LIG4 (DNA ligase 4). In some embodiments, such cancers are characterized by increased alternative end-joining DNA repair. In some embodiments, such cancer types are of epithelial origin, e.g., carcinomas. Accordingly, any one or more of the antibodies or fragments thereof described herein may be used to treat poorly immunogenic tumor (e.g., an “immune-excluded” tumor) sensitized with a cancer therapy aimed to promote T cell immunity, such as chemotherapy, radiation therapy cancer vaccines and oncolytic virus.

Thus, the present disclosure provides combination or adjunct (add-on) cancer therapy comprising a TGFβ inhibitor (e.g., TGFβ1 inhibitor such as Ab6) and a genotoxic agent (e.g., chemotherapeutic agent, radiation therapy, etc.).

According to the present disclosure, a TGFβ inhibitor is used in the treatment of cancer in a subject, wherein the cancer is characterized by increased alternative end-joining DNA repair or impaired double-strand break repair, and wherein the subject receives cancer therapy comprising a genotoxic agent, wherein optionally the cancer therapy comprises chemotherapy and/or radiation therapy. In preferred embodiments, the TGFβ inhibitor is a TGFβ1-selective inhibitor. The present disclosure also provides a genotoxic agent for use in the treatment of cancer in a subject, wherein the cancer is characterized by increased alternative end-joining DNA repair or impaired double-strand break repair, and wherein the subject is treated with a TGFβ inhibitor, wherein optionally the TGFβ inhibitor is a TGFβ1-selective inhibitor. In some embodiments, the genotoxic agent is a chemotherapeutic agent. The present disclosure also provides radiation therapy for use in the treatment of cancer in a subject, wherein the cancer is characterized by increased alternative end-joining DNA repair or impaired double-strand break repair, and wherein the subject is treated with a TGFβ inhibitor, wherein optionally the TGFβ inhibitor is a TGFβ1-selective inhibitor. The present disclosure further provides a TGFβ inhibitor and a cancer therapy comprising chemotherapy or radiation therapy, is used in the treatment of cancer in a subject, wherein the cancer is characterized by increased alternative end-joining DNA repair or impaired double-strand break repair, In any of the embodiments of the combination and adjunct therapies, the subject may be naïve to checkpoint inhibitor therapy. In any of these embodiments of the combination and adjunct therapies, the cancer may be uterine corpus endometrial carcinoma (UCEC), thyroid carcinoma (THCA), testicular germ cell tumors (TGCT), skin cutaneous melanoma (SKCM), prostate adenocarcinoma (PRAD), ovarian serous cystadenocarcinoma (OV), lung squamous cell carcinoma (LUSC), lung adenocarcinoma (LUAD), liver hepatocellular carcinoma (LIHC), kidney renal clear cell carcinoma (KIRC), head and neck squamous cell carcinoma (HNSC), glioblastoma multiforme (GMB), esophageal carcinoma (ESCA), colon adenocarcinoma (COAD), breast invasive carcinoma (BRCA), or bladder urothelial carcinoma (BLCA). In any of these embodiments of the combination and adjunct therapies, the subject may further receive a checkpoint inhibitor therapy.

In immune-excluded tumors where effector T cells are kept away from the site of tumor (hence “excluded”), the immunosuppressive tumor environment may be mediated in a TGFβ1-dependent fashion. These are tumors that are typically immunogenic; however, T cells cannot sufficiently infiltrate, proliferate, and elicit their cytotoxic effects due to the immune-suppressed environment. Typically, such tumors are poorly responsive to cancer therapies such as CBTs. As data provided herein suggest, adjunct therapy comprising a TGFβ1 inhibitor may overcome the immunosuppressive phenotype, allowing T cell infiltration, proliferation, and anti-tumor function, thereby rendering such tumor more responsive to cancer therapy such as CBT.

Thus, the second inquiry is drawn to identification or selection of patients who have immunosuppressive tumor(s), who are likely to benefit from a TGFβ inhibitor therapy, e.g., a TGFβ1 inhibitor such as Ab6. The presence or the degree of frequencies of effector T cells in a tumor is typically indicative of anti-tumor immunity. Therefore, detecting anti-tumor cells such as CD8+ cells in a tumor provides useful information for assessing whether the patient may benefit from a CBT and/or TGFβ1 inhibitor therapy.

Detection may be carried out by known methods such as immunohistochemical analysis of tumor biopsy samples, including digital pathology methods. More recently, non-invasive imaging methods are being developed which will allow the detection of cells of interest (e.g., cytotoxic T cells) in vivo. See for example, imaginab.com/technology/; Tavare et al., (2014) PNAS, 111(3): 1108-1113; Tavare et al., (2015) J Nucl Med 56(8): 1258-1264; Rashidian et al., (2017) J Exp Med 214(8): 2243-2255; Beckford Vera et al., (2018) PLoS ONE 13(3): e0193832; and Tavare et al., (2015) Cancer Res 76(1): 73-82, each of which is incorporated herein by reference. Typically, antibodies or antibody-like molecules engineered with a detection moiety (e.g., radiolabel) can be infused into a patient, which then will distribute and localize to sites of the particular marker (for instance CD8+). In this way, it is possible to determine whether the tumor has an immune-excluded phenotype. If the tumor is determined to have an immune-excluded phenotype, cancer therapy (such as CBT) alone may not be efficacious because the tumor lacks sufficient cytotoxic cells within the tumor environment. Add-on therapy with a TGFβ inhibitor such as those described herein may reduce immuno-suppression thereby rendering the cancer therapy-resistant tumor more responsive to a cancer therapy.

Non-invasive in vivo imaging techniques may be applied in a variety of suitable methods for purposes of diagnosing patients; selecting or identifying patients who are likely to benefit from TGFβ inhibitor therapy, e.g., a TGFβ inhibitor therapy; and/or, monitoring patients for therapeutic response upon treatment. Any cells with a known cell-surface marker may be detected/localized by virtue of employing an antibody or similar molecules that specifically bind to the cell marker. Typically, cells to be detected by the use of such techniques are immune cells, such as cytotoxic T lymphocytes, regulatory T cells, MDSCs, tumor-associated macrophages, NK cells, dendritic cells, and neutrophils. Antibodies or engineered antibody-like molecules that recognize such markers can be coupled to a detection moiety.

Non-limiting examples of suitable immune cell markers include monocyte markers, macrophage markers (e.g., M1 and/or M2 macrophage markers), CTL markers, suppressive immune cell markers, MDSC markers (e.g., markers for G- and/or M-MDSCs), including but are not limited to: CD8, CD3, CD4, CD11 b, CD33, CD163, CD206, CD68, CD14, CD15, CD66b, CD34, CD25, and CD47. In some embodiments, the in vivo imaging comprises T cell tracking, such as cytotoxic CD8-positive T cells. Accordingly, any one of the TGFβ inhibitors of the present disclosure may be used in the treatment of cancer in a subject with a solid tumor, wherein the treatment comprises: i) carrying out an in vivo imaging analysis to detect T cells in the subject, wherein optionally the T cells are CD8+ T cells, and if the solid tumor is determined to be an immune-excluded solid tumor based on the in vivo imaging analysis of step (i), then, administering to the subject a therapeutically effective amount of a TGFβ inhibitor, e.g., Ab6. In some embodiments, the subject has received a CBT, wherein optionally the solid tumor is resistant to the CBT. In some embodiments, the subject is administered with a CBT in conjunction with the TGFβ1 inhibitor, as a combination therapy. The combination may comprise administration of a single formulation that comprises both a checkpoint inhibitor and a TGFβ inhibitor. The TGFβ inhibitor may be a TGFβ1 inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). Alternatively, the combination therapy may comprise administration of a first formulation comprising a checkpoint inhibitor and a second formulation comprising a TGFβ inhibitor, wherein the TGFβ inhibitor may be a TGFβ1 inhibitor, such as a TGFβ1-selective inhibitor, e.g., Ab6, an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, a ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

In some embodiments, the in vivo imaging comprises MDSC tracking, such as G-MDSCs and M-MDSCs. For example, MDSCs may be enriched at a disease site (such as fibrotic tissues and solid tumors) at the baseline. Upon therapy (e.g., TGFβ1 inhibitor therapy), fewer MDSCs may be observed, as measured by reduced intensity of the label (such as radioisotope and fluorescence), indicative of therapeutic effects.

In some embodiments, the in vivo imaging comprises tracking or localization of LRRC33-positive cells. LRRC33-positive cells include, for example, MDSCs and activated M2-like macrophages (e.g., TAMs and activated macrophages associated with fibrotic tissues). For example, LRRC33-positive cells may be enriched at a disease site (such as fibrotic tissues and solid tumors) at the baseline. Upon therapy (e.g., TGFβ1 inhibitor therapy), fewer cells expressing cell surface LRRC33 may be observed, as measured by reduced intensity of the label (such as radioisotope and fluorescence), indicative of therapeutic effects.

In some embodiments, the in vivo imaging comprises the use of PET-SPECT, MRI and/or optical fluorescence/bioluminescence in order to detect target of interest (e.g., molecules or entities which can be bound by the labeled reagent, such as cells and tissues expressing appropriate marker(s)).

In some embodiments, labeling of antibodies or antibody-like molecules with a detection moiety may comprise direct labeling or indirect labeling.

In some embodiments, the detection moiety may be a tracer. In some embodiments, the tracer may be a radioisotope, wherein optionally the radioisotope may be a positron-emitting isotope. In some embodiments, the radioisotope is selected from the group consisting of: ¹⁸F, ¹¹C, ¹³N, ¹⁵O, ⁶⁸Ga, ¹⁷⁷Lu, ¹⁸F and ⁸⁹Zr.

Thus, such methods may be employed to carry out in vivo imaging with the use of labeled antibodies in immune-PET.

In some embodiments, such in vivo imaging is performed for monitoring a therapeutic response to the TGFβ1 inhibition therapy in the subject. For example, the therapeutic response may comprise conversion of an immune excluded tumor into an inflamed tumor, which correlates with increased immune cell infiltration into a tumor. This may be visualized by increased intratumoral immune cell frequency or degree of detection signals, such as radiolabeling and fluorescence. The therapeutic response may additionally or alternatively be a decrease in the level of circulatory MDSCs, such as g-MDSCs (e.g., as determined by flow cytometry). The therapeutic response may additionally or alternatively comprise conversion of an immune desert tumor into an inflamed tumor, which correlates with increased immune cell expansion within the tumor and/or increased cytotoxic T cell function.

Accordingly, the disclosure includes a method for treating cancer which may comprise the following steps: i) selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. In alternative embodiments, the disclosure includes a method for treating cancer which may comprise the following steps: i) selecting a patient diagnosed with cancer comprising a solid tumor, wherein the solid tumor is or is suspected to be an immune infiltrated tumor with an immunosuppressive phenotype (e.g., particularly where the cancer is ccRCC and/or the cancer is resistant to a checkpoint inhibitor therapy); and, ii) administering to the patient an antibody or the fragment encompassed herein in an amount effective to treat the cancer. In some embodiments, the patient has received, or is a candidate for receiving a cancer therapy such as immune checkpoint inhibition therapies (e.g., PD-(L)1 antibodies), chemotherapies, radiation therapies, engineered immune cell therapies, and cancer vaccine therapies. In some embodiments, the selection step (i) comprises detection of immune cells or one or more markers thereof, wherein optionally the detection comprises a tumor biopsy analysis, serum marker analysis, and/or in vivo imaging.

In some embodiments, the patient is diagnosed with cancer for which a CBT has been approved, wherein optionally, statistically a similar patient population with the particular cancer shows relatively low response rates to the approved CBT, e.g., under 25%. For example, the response rates for the CBT may be between about 10-25%, for example about 10-15%. Such cancer may include, for example, ovarian cancer, gastric cancer, and triple-negative breast cancer. The TGFβ inhibitors of the present disclosure may be used in the treatment of such cancer, where the subject has not yet received a CBT. The TGFβ1 inhibitor may be administered to the subject in combination with a CBT. In some embodiments, the subject may receive or may have received additional cancer therapy, such as chemotherapy and radiation therapy (including a radiotherapeutic agent).

In vivo imaging techniques described above may be employed to detect, localize, and/or track certain MDSCs in a patient diagnosed with a TGFβ-associated disease, such as cancer. Healthy individuals have no or low frequency of MDSCs in circulation. With the onset of or progression of such a disease, elevated levels of circulating and/or disease-localized MDSCs may be detected. For example, CCR2-positive M-MDSCs have been reported to accumulate to tissues with inflammation and may cause progression of fibrosis in the tissue (such as pulmonary fibrosis), and this is shown to correlate with TGFβ1 expression. Similarly, MDSCs are enriched in a number of solid tumors (including triple-negative breast cancer) and in part contribute to the immunosuppressive phenotype of the TME. Therefore, treatment response to TGFβ inhibition, such as TGFβ1 inhibition, according to the present disclosure may be monitored by localizing or tracking circulating MDSCs. Reduction of or low frequency of circulating MDSC levels is typically indicative of therapeutic benefits or better prognosis. Accordingly, the current disclosure provides methods of predicting and monitoring therapeutic efficacy of TGFβ inhibitor therapy, e.g., combination therapy of a TGFβ1 inhibitor and a checkpoint inhibitor, by measuring circulating MDSCs in the blood or a blood component of the subject. The current disclosure also provides methods of selecting patients, e.g., patients with immunosuppressive cancers and determining treatment regimens based on levels of circulating MDSCs measured. The TGFβ inhibitor may be a TGFβ1 inhibitor, such as a TGFβ1-selective inhibitor, e.g., Ab6, an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, a ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

The TGFβ inhibitors of the present disclosure may be used in the treatment of cancer in a subject, wherein the cancer is characterized by immune suppression, wherein the cancer optionally comprises a solid tumor that is TGFβ1-positive and TGFβ3-positive. Such subject may be diagnosed with carcinoma. In some embodiments, the carcinoma is breast carcinoma, wherein optionally the breast carcinoma is triple-negative breast cancer (TNBC). Such treatment can further comprise a cancer therapy, including, without limitation, chemotherapies, radiation therapies, cancer vaccines, engineered immune cell therapies (such as CAR-T), and immune checkpoint blockade therapies, such as anti-PD(L)-1 antibodies. The TGFβ inhibitor may be a TGFβ1 inhibitor, such as a TGFβ1-selective inhibitor, e.g., Ab6, or an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, a ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

In some embodiments, a cold tumor is identified, in which few effector cells are present both inside and outside the tumor or is known to be a type of cancer characterized as poorly immunogenic (e.g., a tumor characterized as an immune desert). A subject/patient with such a tumor is treated with an immune-sensitizing cancer therapy, such as chemotherapy, radiation therapy (such as a radiotherapeutic agent), oncolytic viral therapy, and cancer vaccine, in order to elicit stronger T cell response to tumor antigens (e.g., neo-antigens). This step may convert the cold tumor into an “immune excluded” tumor. The subject optionally further receives a CBT, such as anti-PD-(L)1. The subject is further treated with a TGFβ1 inhibitor, such as the antibodies disclosed herein. This may convert the cold or immune excluded tumor into an “inflamed” or “hot” tumor, which confers responsiveness to immunotherapy. Non-limiting examples of poorly immunogenic cancers include breast cancer (such as TNBC), prostate cancer (such as Castration resistant prostate cancer (CRPC)) and pancreatic cancer (such as pancreatic adenocarcinoma (PDAC)).

High affinity, isoform-selective inhibitors of TGFβ1 of the present disclosure, such as Ab6, can inhibit Plasmin-induced activation of TGFβ1. The plasmin-plasminogen axis has been implicated in certain tumorigenesis, invasion and/or metastasis, of various cancer types, carcinoma in particular, such as breast cancer. Therefore, it is possible that the TGFβ inhibitors such as those described herein may exert the inhibitory effects via this mechanism in tumors or tumor models, such as EMT6, involving the epithelia. Indeed, Plasmin-dependent destruction or remodeling of epithelia may contribute to the pathogenesis of conditions involving epithelial injuries and invasion/dissemination of carcinoma. The latter may be triggered by epithelial to mesenchymal transition (“EMT”). It has been reported that plasminogen activation and plasminogen-dependent invasion were more prominent in epithelial-like cells and were partly dictated by the expression of S100A10 and PAI-1 (Bydoun et al., (2018) Scientific Reports, 8:14091).

The disclosure includes a method for selecting a patient population or a subject who is likely to respond to a therapy comprising a TGFβ inhibitor such as those described herein. Subjects selected according to such methods may be the subjects treated according to the various aspects of the present disclosure. Such method may comprise the steps of: providing a biological sample (e.g., clinical sample) collected from a subject, determining (e.g., measuring or assaying) relative levels of TGFβ1, TGFβ2 and TGFβ3 in the sample, and, administering to the subject a composition comprising a TGFβ inhibitor, such as a TGFβ1 inhibitor described herein, if TGFβ1 is the dominant isoform over TGFβ2 and TGFβ3; and/or, if TGFβ1 is significantly overexpressed or upregulated as compared to control. In some embodiments, such method comprises the steps of obtaining information on the relative expression levels of TGFβ1, TGFβ2 and TGFβ3 which was previously determined; identifying a subject to have TGFβ1-positive, preferably TGFβ1-dominant, disease; and administering to the subject a composition comprising a TGFβ inhibitor disclosed herein. In some embodiments, such subject has a disease (such as cancer) that is resistant to a therapy (such as cancer therapy). In some embodiments, such subject shows intolerance to the therapy and therefore has or is likely to discontinue the therapy. Addition of the TGFβ inhibitor to the therapeutic regimen may enable reducing the dosage of the first therapy and still achieve clinical benefits in combination. In some embodiments, the TGFβ inhibitor may delay or reduce the need for surgeries. In some embodiments, the TGFβ inhibitor is a TGFβ1 inhibitor described herein, e.g., Ab6.

Relative levels of the isoforms may be determined by RNA-based assays and/or protein-based assays, which are well-known in the art. In some embodiments, the step of administration may also include another therapy, such as immune checkpoint inhibitors, or other agents provided elsewhere herein. Such methods may optionally include a step of evaluating a therapeutic response by monitoring changes in relative levels of TGFβ1, TGFβ2 and TGFβ3 at two or more time points. In some embodiments, clinical samples (such as biopsies) are collected both prior to and following administration. In some embodiments, clinical samples (such as biopsies) are collected multiple times following treatment to assess in vivo effects over time.

In addition to the above inquiries, the third inquiry interrogates the breadth of TGFβ function, such as TGFβ1 function, involved in a particular disease. In particular, this may be represented by the number of TGFβ1 contexts, namely, which presenting molecule(s) mediate disease-associated TGFβ1 function. TGFβ1-specific, broad-context inhibitors, such as context-independent inhibitors, are advantageous for the treatment of diseases that involve both an ECM component and an immune component of TGFβ1 function. Such disease may be associated with dysregulation in the ECM as well as perturbation in immune cell function or immune response. Thus, the TGFβ1 inhibitors described herein are capable of targeting ECM-associated TGFβ1 (e.g., presented by LTBP1 or LTBP3) as well as immune cell-associated TGFβ1 (e.g., presented by GARP or LRRC33). Such inhibitors inhibit all four of the therapeutic targets (e.g., “context-independent” inhibitors): GARP-associated pro/latent TGFβ1; LRRC33-associated pro/latent TGFβ1; LTBP1-associated pro/latent TGFβ1; and, LTBP3-associated pro/latent TGFβ1, so as to broadly inhibit TGFβ1 function in these contexts.

Whether or not a particular condition of a patient involves or is driven by multiple aspects of TGFβ1 function may be assessed by evaluating expression profiles of the presenting molecules, in a clinical sample collected from the patient. Various assays are known in the art, including RNA-based assays and protein-based assays, which may be performed to obtain expression profiles. Relative expression levels (and/or changes/alterations thereof) of LTBP1, LTBP3, GARP, and LRRC33 in the sample(s) may indicate the source and/or context of TGFβ1 activities associated with the condition. For instance, a biopsy sample taken from a solid tumor may exhibit high expression of all four presenting molecules. For example, LTBP1 and LTBP3 may be highly expressed in CAFs within the tumor stroma, while GARP and LRRC33 may be highly expressed by tumor-associated immune cells, such as Tregs and leukocyte infiltrate, respectively. Similarly, LTBP1 and LTBP3 may be highly expressed in FAFs (e.g., myofibroblasts) within the fibrotic microenvironment, while LRRC33 may be highly expressed by fibrosis-associated immune cells, such as M2 macrophages and MDSCs.

Accordingly, the disclosure includes a method for determining (e.g., testing or confirming) the involvement of TGFβ1 in the disease, relative to TGFβ2 and TGFβ3. In some embodiments, the method further comprises a step of: identifying a source (or context) of disease-associated TGFβ1. In some embodiments, the source/context is assessed by determining the expression of TGFβ presenting molecules, e.g., LTBP1, LTBP3, GARP and LRRC33 in a clinical sample taken from patients. In some embodiments, such methods are performed ex post facto.

With respect to LRRC33-positive cells, Applicant of the present disclosure has recognized that there can be a significant discrepancy between RNA expression and protein expression of LRRC33. In particular, while a select cell type appears to express LRRC33 at the RNA level, only a subset of such cells express the LRRC33 protein on the cell-surface. It is contemplated that LRRC33 expression may be highly regulated via protein trafficking/localization, for example, in terms of plasma membrane insertion and rapid internalization. Therefore, in certain embodiments, LRRC33 protein expression may be used as a marker associated with a diseased tissue (such as tumor tissues) enriched with, for example, activated/M2-like macrophages and MDSCs.

In a related aspect, the present disclosure provides therapeutic use and related treatment methods comprising an immune checkpoint inhibitor, e.g., a PD-(L)1 antibody. Non-limiting examples of useful checkpoint inhibitors include: ipilimumab (Yervoy®); nivolumab (Opdivo®); pembrolizumab (Keytruda®); avelumab (Bavencio®); cemiplimab (Libtayo®); atezolizumab (Tecentriq®); budigalimab (ABBV-181); durvalumab (Imfinzi®), etc.

According to the present disclosure, a cancer treatment method may include a checkpoint inhibitor for use in the treatment of cancer in a subject, wherein the treatment comprises administration of a checkpoint inhibitor to the subject who is treated with a TGFβ inhibitor, wherein, upon treatment of the TGFβ inhibitor, circulating MDSC levels in a sample collected from the subject are reduced, as compared to prior to the treatment. The sample may be a blood sample or a sample of blood component. The checkpoint inhibitor may be a PD-1 antibody. The checkpoint inhibitor may be a PD-L1 antibody. The checkpoint inhibitor may be a CTLA4 antibody. In some embodiments, the checkpoint inhibitor is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®).

According to the present disclosure, a cancer treatment method may include a checkpoint inhibitor for use in the treatment of cancer in a subject who is poorly responsive to the checkpoint inhibitor, or wherein the subject has a cancer with primary resistant to the checkpoint inhibitor, wherein the treatment comprises administering to the subject a TGFβ inhibitor, measuring circulating MDSC levels before and after the administration of the TGFβ inhibitor, and if circulating MDSCs are reduced after the TGFβ inhibitor administration, further administering a checkpoint inhibitor to the subject in an amount sufficient to treat cancer. The checkpoint inhibitor may be a PD-1 antibody. The checkpoint inhibitor may be a PD-L1 antibody. The checkpoint inhibitor may be a CTLA4 antibody. In some embodiments, the checkpoint inhibitor is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®). Optionally, the TGFβ inhibitor is an isoform-selective inhibitor of TGFβ1, wherein optionally the inhibitor is an activation inhibitor of TGFβ1 or neutralizing antibody that selectively binds TGFβ1; or an isoform-non-selective inhibitor (e.g., inhibitors of TGFβ1/2/3, TGFβ1/3, TGFβ1/2).

In some embodiments, an effective amount of TGFβ inhibitor is used to treat cancer (e.g., carcinoma) in a patient, wherein no checkpoint inhibitor is approved for the treatment of the cancer. Preferably, the TGFβ inhibitor is a TGFβ1-selective inhibitor, wherein optionally the TGFβ1-selective inhibitor is Ab6 or a variant thereof. The TGFβ inhibitor may be used as a monotherapy or used in conjunction with an approved cancer therapy, such as chemotherapy and radiation therapy.

In some embodiments, Ab6 may be used as monotherapy to treat cancer such as ovarian cancer, colorectal cancer, and prostate cancer, in a patient. The effective amount of Ab6 may be an amount sufficient to stabilize disease (SD), e.g., the observed change in tumor size is below the progressive disease (PD) and above the partial response (PR) levels according to the RECIST response evaluation criteria in solid tumors. In some embodiments, the effective amount is between 240-3000 mg per dose, administered every 2 weeks or every 3 weeks. For example, the patient has ovarian cancer and is dosed at 240 mg per dose every 3 weeks, at 800 mg per dose every 3 weeks, 1600 mg per dose every 3 weeks, or 2400 mg per dose every 3 weeks. In some embodiments, the patient is a candidate for further receiving a genotoxic therapy such as chemotherapy and radiation therapy.

In some embodiments, Ab6 may be used as combination therapy or adjunct therapy to treat cancer such as renal cell carcinoma, liver cancer and oropharynx cancer, in a patient. The effective amount of Ab6 may be an amount sufficient to achieve partial response (PR) or disease stabilization (SD) according to the RECIST response evaluation criteria in solid tumors. In some embodiments, the effective amount of Ab6 is between 240-3000 mg per dose, administered every 2 weeks or every 3 weeks, in conjunction with a checkpoint inhibitor therapy, such as anti-PD-1 antibody and anti-PD-L1 antibody. For the combination/adjunct therapy, the dosing regimen approved for a particular checkpoint inhibitor can be followed. In some embodiments, the patient is a CPI-naïve patient (who has never received a CPI). For example, the patient has a renal cell carcinoma and is dosed at 800 mg of Ab6 every 3 weeks in conjunction with anti-PD-1 (e.g., Pembro at 200 mg every 3 weeks). The poartial response (PR) may comprise 50% or greater reduction in tumor size (volume) relative to baseline. In some embodiments, the patient has a history of primary nonresponse to the checkpoint inhibitor therapy (alone or in combination with other therapy). For example, the patient may have had disease progression (DP) on prior checkpoint inhibitor therapy, such as anti-PD-(L)1.

### Patient Selection

In various embodiments, the present disclosure provides anti-TGFβtreatments of cancer in specific patient populations, such as those exhibiting certain biomarkers indicating they are likely to be responsive to anti-TGFβtreatments and/or those who receives certain comitant therapies such as administration of genotoxic agents or other treatments that induce reactive oxygen species. In some embodiments, the patient has CD8+ T cell-infiltrated tumor(s). In some embodiments, the patient has CD8+ T cell-infiltrated tumor(s) with a high Treg/CD8+ T cell ratio. In some embodiments, the patient has heightened levels of circulating MDSCs. In some embodiments, the cancer is a carcinoma, wherein optionally, the carcinoma has undergone ETM (e.g., has a mesenchymal phenotype). In some embodiments, the carcinoma is RCC, such as ccRCC. In some embodiments, the RCC is infiltrated with CD8+ T cells.

In some embodiments, a patient who receives certain therapies may benefit from anti-TGFβtreatments given at particular time points. Such patients may be patients who are candidates for receiving such therapies, patients scheduled to receive such therapies, patients about to receive such therapies, or patients who are receiving such therapies. For instance, a patient who receives genotoxic agents such as radiation therapy and chemotherapy may first receive anti-TGFβtreatments (such as any of the TGFβ1 antibodies disclosed herein) weeks to hours before receiving the radiation. In some embodiments, a patient receives the anti-TGFβtreatment 4 weeks, 3 weeks, 2 weeks, 1 week, 6 days, 5 days, 4 days, 3 days, 2 days, 1 day, or 23 hours 22 hours, 21 hours, 20 hours, 19 hours 18 hours, 17 hours, 16 hours, 15 hours, 14 hours, 13 hours, 12 hours, 11 hours, 10 hour, 9 hours, 8 hours, 7 hours, 6 hours, 5 hours, 4 hours, 3 hours, 2 hours or 1 hour before receiving the radiation therapy. Nonlimiting examples of genotoxic agents include 5-FU, paclitaxel, cisplatin, bleomycin, gemtuzumab ozogamicin, L-Phenylalanine, bortezomib, cladribine, carmustine, amsacrine, chlorambucil, raltitrexed, mitomycin, bexarotene, vindesine, floxuridine, thioguanine, vinorelbine, gemcitabine, teniposide, oxaliplatin, cyclophosphamide, pentostatin, methotrexate, vinblastine, pemetrexed, daunorubicin, irinotecan, etoposide, dacarbazine, temozolomide, azacitidine, carboplatin, dactinomycin, cytarabine, doxorubicin, hydroxyurea, busulfan, topotecan, mercaptopurine, melphalan, fludarabine, capecitabine, arsenic trioxide, idarubicin, ifosfamide, mitoxantrone, lomustine, docetaxel, decitabine, nelarabine, thiotepa, trabectedin, emtansine, bendamustine, cabazitaxel, and eribulin, that induce reactive oxygen species. Similar schedules may be afforded to other treatments. Without being bound by theory, such prior administration may serve to bind available TGFβ1 and/or provide a high serum concentration of antibody to engage TGFβ1 and prevent activation by reactive oxygen species.

In various embodiments, a patient with cancer is selected for treatment based on the presensce of one or more biomarkers indicative that the patient may benefit from receiving a TGFβ1 inhibitor, e.g., a treatment as disclosed herein. In some embodiments, detection of one or more biomarkers in the cancer of a patient is performed to determine whether the patient is suitable for the TGFβ treatments disclosed herein. Non-limiting examples of such biomarkers include increased TGFβ1 transcript and/or protein levels in a tumor sample or in a fluid (e.g., serum), TGFβ1 activators (e.g., integrins and proteases), ROS markers, ECM markers, CAF markers, epithelial markers, mesenchymal markers, immunosuppression markers, SMAD4 gene, transcripts and proteins, MTAP gene, transcripts, and proteins, IFNγ, and circulating MDSCs. In some embodiments, detection of the one or more biomarkers is performed prior to administration of a TGFβ treatment to the patient. In some embodiments, optionally, detection of a change in the one or more biomarkers in the patient after one or more rounds of the TGFβ treatment is performed to determine the efficacy of the treatment. In some embodiments, the patient has received prior therapies for the cancer, including checkpoint inhibitor therapies and/or genotoxic agents (chemotherapies and/or radiation therapies). In some embodiments, the patient further receives an additional treatment together with the TGFβ treatments. In some embodiments, the patient starts a TGFβ treatment prior to initiating an additional treatment. In some embodiments, the patient starts a TGFβ treatment either prior to or after initiating an additional treatment. In some embodiments, the patient starts a TGFβ treatment either prior to or after initiating an additional treatment and continue to receive the TGFβ treatment together with the additional treatment. In some embodiments, the patient starts a TGFβ treatment either prior to or after initiating an additional treatment, then receives the TGFβ treatment together with the additional treatment, and continues to receive the TGFβ treatment after the additional treatment ends. In some embodiments, the patient receives an additional treatment such as checkpoint inhibitor therapies and/or genotoxic agents (e.g., chemotherapies and/or radiation therapies). In some embodiments, the TGFβ treatments comprise administration a TGFβ1-selective inhibitor to the patient. In some embodiments, the TGFβ1-selective inhibitor is SRK-181.

In some embodiments, the patient is selected for a TGFβ treatment when increased levels of TGFβ1 transcripts and/or proteins are detected in the cancer and/or in a fluid sample from the patient.

In some embodiments, the patient is selected for a TGFβ treatment when increased levels of one or more TGFβ1 activators are detected in the cancer and/or in a fluid sample from the patient. In some embodiments, the TGFβ1 activator comprises one or more integrins capable of binding an RGD motif, e.g., wherein the integrin and comprises alpha-v, alpha-5, alpha-11, beta-6, beta-8, etc. In some embodiments, the TGFβ1 activator comprises one or more proteases such as kallikrein, chemotrypsin, trypsin, elastase, plasmin, thrombin, zinc metalloproteases (MMP), ADAM proteases, etc.

In some embodiments, the patient is selected for the TGFβ treatment when increase in one or more ROS marker is detected in the patient, indicating an increase in ROS production. In some embodiments, an ROS marker comprises one or more of isoprostanes (IsoPs), malondialdehyde (MDA), nitrotyrosine, S-glutathionylation, myeloperoxidase (MPO), oxidized low-density lipoprotein (OxLDL), antioxidant enzymes, etc. (Ho et al., Redox Biol, 2013 Oct. 8, 1(1):483-491). In some embodiments, the patient is receiving one or more genotoxic therapies such as chemotherapies and radiation therapies. Genotoxic therapies are known to increase ROS production, which would activate TGFβ signaling. In some embodiments, the patient receives the TGFβ treatments prior to and/or concurrently with their genotoxic therapies. Non-limiting examples of chemotherapy agents include 5-FU, paclitaxel, cisplatin, and bleomycin. Other factors that may lead to release of ROS include hypoxia, fatty acid and/or bile acid build up, increase of TNF-α, p53 expression and accumulation, an/or increase in anti-apoptotic proteins such as Bax and/or other Bcl-2 family members (Kennel et al., Redox Biol, 2021 June, 42:101891).In some embodiments, the patient is selected for a TGFβ treatment when an increased deposition of extracellular matrix (ECM) is detected in the cancer. In some embodiments, the increased deposition of ECM is indicated by the levels of one or more ECM markers like collagen, fibronectin, and/or fibrillin and/or by the levels of one or more cancer-associated fibroblast (CAF) markers such as actin alpha, platelet-derived growth factor receptor alpha (PDGFRα/CD140a), platelet-derived growth factor receptor beta (PDGFRβ/CD140b), fibroblast specific protein 1 (FSP-1/S100A4), fibroblast activation protein (FAP), nicotinamide N-methyltransferase (NNMT), etc.

In some embodiments, the patient is selected for a TGFβ treatment when decrease in the level of one or more epithelial markers and/or increase in the level of one or more mesenchymal markers is detected in the cancer, Non-limiting examples of epithelial markers include E-cadherin, a-catenin, y-catenin, and cytokeratin, Non-limiting examples of mesenchymal markers include fibronectin, vimentin, and N-cadherin.

In some embodiments, the patient is selected for a TGFβ treatment when elevated levels of one or more Treg immunosuppression markers (e.g., CD4, FOXP3, and CD25) and/or LRRC33 are detected in the cancer. In some embodiments, the patient is selected for a TGFβ treatment when there is an increased number of Treg cells in the cancer. In some embodiments, the cancer is ccRCC and the elevated levels of markers and/or the increased number of Treg cells is detected prior to treatment (Liu et al., Front Immunol, 2022 Jun. 24, 13:791158).In some embodiments, the level of CD8+ cells infiltrating the tumor(s) in the patient is determined prior to administration of a TGFβ treatment. In some embodiments, a patient is selected for a TGFβ treatment of ccRCC when the number of CD8+ cells infiltrating the tumor(s) is higher than that in the normal tissue(s). In some embodiments, the ratio of Treg/CD8+ T cell in the tumor is determined prior to administration of a TGFβ treatment. Without being bound by theory, tumors with a higher Treg/CD8+ T cell ratio would less likely to respond to checkpoint inhibitor therapies.

Platelets are found to be enriched in TMEs and possibly contributing to immunosuppressive phenotypes (Liu et al., Front Immunol, 2022 Jun. 24, 13:791158). In some embodiments, the patient is selected for a TGFβ treatment when there is an elevated level of platelets in the cancer.

In some embodiments, the patient is selected for a TGFβ treatment when the cancer is SMAD4-deficient. In some embodiments, the cancer is SMAD4-null. In some embodiments, the cancer has decreased SMAD4 transcripts and/or SMAD4 proteins. Without being bound by theory, such patients may be selected for a TGFβ treatment as the oncogenic effects of TGFβ can be mediated by SMAD2/3 in the absence of SMAD4, suggesting that such patients may have upregulated TGFβ (Bertrand-Chapel et al., Commun Biol, 2022 Oct. 7; 5(1):1068).

In some embodiments, a patient is selected for a TGFβ treatment when the cancer is methylthioadenosine phosphorylase (MTAP)-deficient. In some embodiments, the cancer is MTAP-null. In some embodiments, the cancer has a deletion in the 9p21 locus. In some embodiments, the cancer may further contain additional deletion(s) of tumor suppressor gene(s), e.g., cyclin dependent kinase inhibitor 2A (CDKN2A). In some embodiments, the cancer has reduced expression of MTAP. In some embodiments, mutant MTAP with decreased activity is produced in the cancer. MTAP deletin has been associated with deletion of CDKN2A loss in the 9p21 chromosomal locus and linked to poor response to cancer immunotherapy (Hu et al., Cancer Res, 2021 Oct. 1, 81(19):4964-4980; Alhalabi et al., Nat Commun, 2022 Apr. 4, 13(1):1797). Without being bound by theory, TGFβ treatments disclosed herein may counteract the resistance to immunotherapy in these patients by reversing the immune desertification resulting from MTAP loss (Fan et al., Front Cell Dev Biol, 2023 Apr. 5, 11:1173356).

In some embodiments, the patient is selected for a TGFβ treatment when the cancer has low levels of IFNγ, perforin, and/or granzyme A/B. In some embodiments, the patient has RCC, such as ccRCC. Tumors infiltrated by CD8+ T cells expressing reduced amounts of cytotoxic enzymes, such as IFNγ, perforin, and granzyme A/B, are less likely to respond to checkpoint inhibitor therapies. Combination therapies that block both PD-L1 and TGFβ signaling pathways may activate IFNγ signaling in NSCLC (Bauer et al., J Immunother Cancer, 2023 Nov. 29, 11(11):e007353). Similar combination therapies also increase IFNγ-effector T cells in tumors, which promotes tumor clearance (Castiglioni et al., Nat Commun, 2023 Aug. 5; 14(1):4703).

In some embodiments, the patient is selected for a TGFβ treatment when the patient has a low level of circulating MDSCs. For example, a ten-fold increase in MDSCs was detected in breast cancer patients in one study when compared to healthy control, suggesting a potential selection criteria for patients who may respond to treatment with a TGFβ inhibitor as MDSC levels may be indicative of immune exclusion phenotype. [REF] International Patent Application Nos. WO 2021/142448 and WO 2022/204581, which disclose markers and methods for detecting and quantifying circulating MDSCs, are incorporated by reference herein in their entirety.

The detected increase in a biomarkers disclosed herein may be at least 1.5, at least 2.0, at least 2.5, at least 3, at least 5, or at least 10 folds. The detected decrease in the biomarkers disclosed herein may be a decrease by at least 10%, at least 15%, at least 20%, at least 25%, at least 30%, at least 50%, at least 70%, or at least 90%.

### Combination Therapy

Disclosed herein are pharmaceutical compositions of a TGFβ inhibitor, e.g., an antibody or antigen-binding portion thereof, described herein, and related methods used as, or referring to, combination therapies for treating subjects who may benefit from TGFβ inhibition in vivo. In any of these embodiments, such subjects may receive combination therapies that include a first composition comprising at least one TGFβ inhibitor, e.g., Ab6, in conjunction with at least a second composition comprising at least one additional therapeutic intended to treat the same or overlapping disease or clinical condition. In some embodiments, such subjects may receive an additional third composition comprising at least one additional therapeutic intended to treat the same or overlapping disease or clinical condition. The TGFβ inhibitor may be a TGFβ1 inhibitor, such as a TGFβ1-selective inhibitor (e.g., one which does not inhibit TGFβ2 and/or TGFβ3 signaling at a therapeutically effective dose), e.g., Ab6, or an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). The first, second, and third compositions may both act on the same cellular target, or discrete cellular targets. In some embodiments, the first, second, and third compositions may treat or alleviate the same or overlapping set of symptoms or aspects of a disease or clinical condition. In some embodiments, the first, second, and third compositions may treat or alleviate a separate set of symptoms or aspects of a disease or clinical condition. In some embodiments, the combination therapy may comprise more than three compositions, which may act on the same target or discrete cellular targets, and which may treat or alleviate the same or overlapping set of symptoms or aspects of a disease or clinical condition. To give but one example, the first composition may treat a disease or condition associated with TGFβ signaling, while the second composition may treat inflammation or fibrosis associated with the same disease, etc. As another example, the first composition may treat a disease or condition associated with TGFβ signaling, while the second and third compositions may have anti-neoplastic effects and/or help reverse immune suppression. In certain embodiments, the first composition may be a TGFβ inhibitor (e.g., a TGFβ1 inhibitor described herein), the second composition may be a checkpoint inhibitor, and the third composition may be a checkpoint inhibitor distinct from the second composition. In certain embodiments, a first composition comprising a TGFβ inhibitor (e.g., a TGFβ1 inhibitor described herein) is combined with a checkpoint inhibitor and a chemotherapeutic agent. In certain embodiments, a first composition comprising a TGFβ inhibitor (e.g., a TGFβ1 inhibitor described herein) is combined with two distinct checkpoint inhibitors and a chemotherapeutic agent. Such combination therapies may be administered in conjunction with each other. As noted above, the phrase “in conjunction with,” in the context of combination therapies, means that therapeutic effects of a first therapy overlap temporally and/or spatially with therapeutic effects of a second therapy in the subject receiving the combination therapy. The first, second, and/or additional compositions may be administered concurrently (e.g., simultaneously), separately, or sequentially. Thus, the combination therapies may be formulated as a single formulation for concurrent or simultaneous administration, or as separate formulations for concurrent (e.g., simultaneous), separate, or sequential administration of the therapies. As used herein, a combination therapy may comprise two or more therapies (e.g., compositions) given in a single bolus or administration, or in a single patient visit (e.g., to or with a medical professional) but in two or more separate boluses or administrations, or in separate patient visits (and, e.g., in two or more separate boluses or administrations). For instance, the therapies may be given less than about 5 minutes apart, or 1 minute apart. The therapies may be given less than about 30 minutes or 1 hour apart (e.g., in a single patient visit). In some embodiments, the therapies may be given more than about 1 minute, about 2 minutes, about 5 minutes, about 10 minutes, about 15 minutes, about 30 minutes, about 45 minutes, about 1 hour, about 2 hour, about 4 hours, about 6 hours, about 8 hours, about 10 hours, about 1 day, about 2 day, about 3 days, about 5 days, about 1 week, about 2 weeks, about 3 weeks, about 1 month, or more, apart. In some embodiments, the therapies may be given more than about 1 day apart (e.g., in separate visits). The therapies may be given within 3 months (e.g., within 1 month) of one another. In some embodiments, a therapy may be given according to the dosing schedule of one or more approved therapeutics for treating the condition (e.g., administered at the same frequency as for an approved checkpoint inhibitor or other chemotherapeutic agent).

In certain embodiments, the TGFβ inhibitor (e.g., a TGFβ1 inhibitor described herein) may be administered in an amount of about 3000 mg, 2400 mg, 2000 mg, 1600 mg, 800 mg, 240 mg, 80 mg, or less. In certain embodiments, the TGFβ inhibitor (e.g., Ab6) may be administered in an amount of about 3000 mg, 2400 mg, 2000 mg, 1600 mg, 800 mg, 240 mg, 80 mg, or less, e.g., at a frequency of once every two weeks, three weeks, or any multiples of two weeks or three weeks (e.g., once every four weeks, once every six weeks), wherein the TGFβ inhibitor (e.g., Ab6) is administered alone or in combination with a checkpoint inhibitor therapy, (e.g., any approved checkpoint inhibitor therapy, including, but not limited to, antibodies or other agents against cytotoxic T-lymphocyte antigen-4 (CTLA-4), programmed cell death protein 1 (PD-1), programmed cell death receptor ligand 1 (PD-L1), T-cell immunoglobulin domain and mucin domain-3 (TIM3), lymphocyte-activation gene 3 (LAG3), killer cell immunoglobulin-like receptor (KIR), glucocorticoid-induced tumor necrosis factor receptor (GITR), or V-domain immunoglobulin (Ig)-containing suppressor of T-cell activation (VISTA)).

In certain embodiments, the TGFβ inhibitor (e.g., Ab6) may be administered alone at 3000 mg once every six weeks. In certain embodiments, the TGFβ inhibitor (e.g., Ab6) may be administered at 3000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every six weeks. In certain embodiments, the TGFβ inhibitor (e.g., Ab6) may be administered at 2400 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every six weeks. In certain embodiments, the TGFβ inhibitor (e.g., Ab6) may be administered at 2000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every six weeks. In certain embodiments, the TGFβ inhibitor (e.g., Ab6) may be administered at 1600 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every six weeks. In certain embodiments, the TGFβ inhibitor (e.g., Ab6) may be administered at 800 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every six weeks. In certain embodiments, the TGFβ inhibitor (e.g., Ab6) may be administered at 240 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every six weeks. In certain embodiments, the TGFβ inhibitor (e.g., Ab6) may be administered at 80 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every six weeks. In certain embodiments, the TGFβ inhibitor (e.g., Ab6) may be administered at an amount of less than 80 mg once every six weeks, wherein the TGFβ inhibitor (e.g., Ab6) is administered in combination with an anti-PD-(L)1 therapy. In some embodiments, the TGFβ inhibitor (e.g., Ab6) may be administered every six weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every four weeks. In certain embodiments, the TGFβ inhibitor (e.g., Ab6) may be administered at 3000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every four weeks. In certain embodiments, the TGFβ inhibitor (e.g., Ab6) may be administered at 2400 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every four weeks. In certain embodiments, the TGFβ inhibitor (e.g., Ab6) may be administered at 2000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every four weeks. In certain embodiments, the TGFβ inhibitor (e.g., Ab6) may be administered at 1600 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every four weeks. In certain embodiments, the TGFβ inhibitor (e.g., Ab6) may be administered at 800 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every four weeks. In certain embodiments, the TGFβ inhibitor (e.g., Ab6) may be administered at 240 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every four weeks. In certain embodiments, the TGFβ inhibitor (e.g., Ab6) may be administered at 80 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every four weeks. In certain embodiments, the TGFβ inhibitor (e.g., Ab6) may be administered at an amount of less than 80 mg once every four weeks, wherein the TGFβ inhibitor (e.g., Ab6) is administered in combination with an anti-PD-(L)1 therapy. In some embodiments, the TGFβ inhibitor (e.g., Ab6) may be administered every four weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every three weeks. In certain embodiments, the TGFβ inhibitor (e.g., Ab6) may be administered at 3000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every three weeks. In certain embodiments, the TGFβ inhibitor (e.g., Ab6) may be administered at 2400 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every three weeks. In certain embodiments, the TGFβ inhibitor (e.g., Ab6) may be administered at 2000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every three weeks. In certain embodiments, the TGFβ inhibitor (e.g., Ab6) may be administered at 1600 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every three weeks. In certain embodiments, the TGFβ inhibitor (e.g., Ab6) may be administered at 800 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every three weeks. In certain embodiments, the TGFβ inhibitor (e.g., Ab6) may be administered at 240 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every three weeks. In certain embodiments, the TGFβ inhibitor (e.g., Ab6) may be administered at 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every three weeks. In certain embodiments, the TGFβ inhibitor (e.g., Ab6) may be administered at an amount of less than 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of three weeks, wherein the TGFβ inhibitor (e.g., Ab6) is administered in combination with an anti-PD-(L)1 therapy. In some embodiments, the TGFβ inhibitor (e.g., Ab6) may be administered every three weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every two weeks. In certain embodiments, the TGFβ inhibitor (e.g., Ab6) may be administered at 3000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every two weeks. In certain embodiments, the TGFβ inhibitor (e.g., Ab6) may be administered at 2400 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every two weeks. In certain embodiments, the TGFβ inhibitor (e.g., Ab6) may be administered at 2000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every two weeks. In certain embodiments, the TGFβ inhibitor (e.g., Ab6) may be administered at 1600 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every two weeks. In certain embodiments, the TGFβ inhibitor (e.g., Ab6) may be administered at 800 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every two weeks. In certain embodiments, the TGFβ inhibitor (e.g., Ab6) may be administered at 240 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every two weeks. In certain embodiments, the TGFβ inhibitor (e.g., Ab6) may be administered at 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every two weeks. In certain embodiments, the TGFβ inhibitor (e.g., Ab6) may be administered at an amount of less than 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of two weeks, wherein the TGFβ inhibitor (e.g., Ab6) is administered in combination with an anti-PD-(L)1 therapy. In some embodiments, the TGFβ inhibitor (e.g., Ab6) may be administered every two weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, a TGFβ inhibitor (e.g., a TGFβ1 inhibitor described herein, e.g, Ab6) may be administered in combination with checkpoint inhibitor therapy, e.g., an anti-PD-(L)1 therapy), to a subject who is a non-responder to checkpoint inhibitor therapy, e.g., an anti-PD-(L)1 therapy). In certain embodiments, the subject has not previously received a checkpoint inhibitor therapy. Exemplary checkpoint inhibitors include, but are not limited to, nivolumab (Opdivo®, anti-PD-1 antibody), pembrolizumab (Keytruda®, anti-PD-1 antibody), cemiplimab (Libtayo®, anti-PD-1 antibody), budigalimab (ABBV-181, anti-PD-1 antibody); BMS-936559 (anti-PD-L1 antibody), atezolizumab (Tecentriq®, anti-PD-L1 antibody), avelumab (Bavencio®, anti-PD-L1 antibody), durvalumab (Imfinzi®, anti-PD-L1 antibody), ipilimumab (Yervoy®, anti-CTLA4 antibody), tremelimumab (anti-CTLA4 antibody), IMP-321 (eftilgimod alpha or “ImmuFact®”, anti-LAG3 large molecule), BMS-986016 (Relatlimab, anti-LAG3 antibody), and lirilumab (anti-KIR2DL-1, -2, -3 antibody).

In certain embodiments, the TGFβ inhibitor (e.g., Ab6) is administered alone to a subject having advanced solid cancer. In certain embodiments, the TGFβ inhibitor (e.g., Ab6) is administered in combination with a checkpoint inhibitor therapy to a subject having advanced solid cancer. In certain embodiments, the TGFβ inhibitor (e.g., Ab6) is administered in combination with a checkpoint inhibitor therapy to a subject having advanced solid cancer, wherein the subject is a non-responder to prior checkpoint inhibitor therapy. In some embodiments, the subject has non-small cell lung cancer (NSCLC), melanoma (MEL), or urothelial carcinoma (UC), including metastatic urothelial carcinoma (mUC). In some embodiments, the subject has ovarian cancer, colorectal cancer (CRC), bladder cancer, renal cell carcinoma (RCC), including clear cell RCC, papillary RCC, chromophobe RCC, collecting duct RCC, or unclassified RCC, or head and neck cancer (e.g., head and neck squamous cell carcinoma (HNSCC) or oropharynx cancer)). In some embodiments, the subject has esophageal cancer, gastric cancer, hepatocellular carcinoma (HCC), triple-negative breast cancer (TNBC), cervical cancer, endometrial cancer, basal cell carcinoma (BCC), cutaneous squamous cell carcinoma (CSCC), merkel cell carcinoma (MCC), small-cell lung cancer (SCLC), primary mediastinal large B-cell lymphoma (PMBCL), Hodgkin's lymphoma, microsatellite instability high cancer (MSI-H) (e.g., MSI-H CRC), mismatch repair deficient cancer (dMMR)(e.g., dMMR CRC), tumor mutational burden-high (TMB-H) cancer, or malignant pleural mesothelioma (MPM). In certain embodiments, the TGFβ inhibitor (e.g., Ab6) is administered in combination with a checkpoint inhibitor therapy to a subject having urothelial carcinoma (UC), including metastatic urothelial carcinoma (mUC), melanoma (MEL), or non-small cell lung cancer NSCLC. In certain embodiments, the subject is a non-responder to checkpoint inhibitor therapy. In certain embodiments, the checkpoint inhibitor therapy is pembrolizumab (e.g., Keytruda®). In certain embodiments, the checkpoint inhibitor therapy is nivolumab (e.g., Opdivo®). In certain embodiments, the checkpoint inhibitor therapy is cemiplimab (e.g., Libtayo®). In certain embodiments, the checkpoint inhibitor therapy is atezolizumab (e.g., Tecentriq®). In certain embodiments, the checkpoint inhibitor therapy is avelumab (e.g., Bavencio®). In certain embodiments, the checkpoint inhibitor therapy is durvalumab (e.g., Imfinzi®). In certain embodiments, the checkpoint inhibitor therapy is budigalimab (ABBV-181).

In certain embodiments, the TGFβ inhibitor (e.g., Ab6) is administered at 3000 mg, 2400 mg, 2000 mg, 1600 mg, 800 mg, 240 mg, 80 mg, or less in combination with pembrolizumab at a frequency of once every two weeks, three weeks, or any multiples of two weeks or three weeks (e.g., once every four weeks, once every six weeks). In certain embodiments, the TGFβ inhibitor (e.g., Ab6) is administered in combination with pembrolizumab to a subject having NSCLC, UC, MEL, esophageal cancer, gastric cancer, HNSCC, HCC, cervical cancer, SCLC, PMBCL, Hodgkin's lymphoma, MSI-H or dMMR cancer, or TMB-H cancer. In certain embodiments, the subject is a non-responder to pembrolizumab. In certain embodiments, the subject has not received pembrolizumab previously. In certain embodiments, the TGFβ inhibitor (e.g., Ab6) is administered in combination with pembrolizumab to a subject having NSCLC who is a non-responder to pembrolizumab treatment. In certain embodiments, the subject having NSCLC has not received pembrolizumab previously. In certain embodiments, the TGFβ inhibitor (e.g., Ab6) is administered in combination with pembrolizumab to a subject having MEL who is a non-responder to pembrolizumab treatment. In certain embodiments, the subject having MEL has not received pembrolizumab previously. In certain embodiments, the TGFβ inhibitor (e.g., Ab6) is administered in combination with pembrolizumab to a subject having UC or mUC who is a non-responder to pembrolizumab treatment. In certain embodiments, the subject having UC or mUC has not received pembrolizumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with nivolumab at a frequency of once every two weeks, three weeks, or any multiples of two weeks or three weeks (e.g., once every four weeks, once every six weeks). In certain embodiments, the TGFβ inhibitor (e.g., Ab6) is administered in combination with nivolumab to a subject having NSCLC, UC, MEL, esophageal cancer, HNSCC, HCC, RCC, Hodgkin's lymphoma, MSI-H or dMMR CRC, or MPM. In certain embodiments, the subject is a non-responder to nivolumab. In certain embodiments, the subject has not received nivolumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with cemiplimab at a frequency of once every two weeks, three weeks, or any multiples of two weeks or three weeks (e.g., once every four weeks, once every six weeks). In certain embodiments, the TGFβ inhibitor (e.g., Ab6) is administered in combination with cemiplimab to a subject having BCC or CSCC. In certain embodiments, the subject is a non-responder to cemiplimab. In certain embodiments, the subject has not received cemiplimab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with atezolizumab at a frequency of once every two weeks, three weeks, or any multiples of two weeks or three weeks (e.g., once every four weeks, once every six weeks). In certain embodiments, the TGFβ inhibitor (e.g., Ab6) is administered in combination with atezolizumab to a subject having NSCLC, MEL, HCC, TNBC, or SCLC. In certain embodiments, the subject is a non-responder to atezolizumab. In certain embodiments, the subject has not received atezolizumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with avelumab at a frequency of once every two weeks, three weeks, or any multiples of two weeks or three weeks (e.g., once every four weeks, once every six weeks). In embodiments, the TGFβ inhibitor (e.g., Ab6) is administered in combination with avelumab to a subject having UC or MCC. In certain embodiments, the subject is a non-responder to avelumab. In certain embodiments, the subject has not received avelumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with durvalumab at a frequency of once every two weeks, three weeks, or any multiples of two weeks or three weeks (e.g., once every four weeks, once every six weeks). In embodiments, the TGFβ inhibitor (e.g., Ab6) is administered in combination with durvalumab to a subject having NSCLC or SCLC. In certain embodiments, the subject is a non-responder to durvalumab. In certain embodiments, the subject has not received durvalumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181), e.g., at a dose of 250 mg, 375 mg, or 500 mg at a frequency of once every two weeks, once every three weeks, or any multiples of two weeks or three weeks (e.g., once every four weeks, once every six weeks). In some embodiments, the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181) wherein budigalimab is administered at a frequency of once every two weeks at a dose of 250 mg. In some embodiments, the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181), wherein budigalimab is administered at a frequency of once every three weeks at a dose of 375 mg. In some embodiments, the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181), wherein budigalimab is administered at a frequency of once every four weeks at a dose of 500 mg. In certain embodiments, the TGFβ inhibitor (e.g., Ab6) is administered in combination with budigalimab to a subject having a locally advanced or metastatic solid tumor. See, e.g., NCT 03821935 (Study to determine the safety, tolerability, pharmacokinetics and recommended phase 2 dose (RP2D) of ABBV-151 as a single agent and in combination with ABBV-181 in participants with locally advanced or metastatic solid tumors. https://clinicaltrials.gov/ct2/show/NCT03821935). In certain embodiments, budigalimab is administered once every four weeks. In certain embodiments, the TGFβ inhibitor (e.g., Ab6) is administered in combination with budigalimab to a subject having triple-negative breast cancer (TNBC), pancreatic adenocarcinoma, urothelial cancer, or Hepatocellular carcinoma (HCC). In certain embodiments, the TGFβ inhibitor (e.g., Ab6) is administered in combination with budigalimab to a subject having non-small cell lung cancer (NSCLC) or head and neck squamous cell carcinoma. Italiano et al. Cancer Immunol Immunother. 2022 February; 71(2):417-431. In certain embodiments, the subject is a non-responder to budigalimab. In certain embodiments, the subject has not received budigalimab previously.

In certain embodiments, where the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181) budigalimab is administered once every two weeks. In further embodiments, budigalimab is administered once every two weeks at a dose of 250 mg. In certain embodiments, where the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181) budigalimab is administered once every three weeks. In further embodiments, budigalimab is administered once every three weeks at a dose of 375 mg. In certain embodiments, where the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181) budigalimab is administered once every four weeks. In further embodiments, budigalimab is administered once every four weeks at a dose of 500 mg.

### Resistance to Genotoxic Agents

In addition to the role of TGFβ1 in mediating primary checkpoint inhibitor resistance, TGFβ1 may also contribute to antitumor immunity related to genotoxic therapies, such as chemotherapy and radiation therapy.

For example, proteomic analysis suggests that the TGFβ pathway may contribute to chemo-resistance in ovarian cancer. For example, it has been reported that progression-free survival in patients with ovarian cancer correlated with CA125 read (Ca-125 normalization by cycle 3 chemo) (ncbi.nlm.nih.gov/pmc/articles/PMC2877659/pdf/ukmss).

Another paper hypothesized that TGFβ signaling may increase occurrence of breast cancer stem cells that contribute to chemotherapy resistance (ncbi.nlm.nih.gov/pmc/articles/PMC3582135). In that study, the authors compared primary breast cancer biopsies before and after chemotherapy and observed increased gene expression associated with TGFβ signaling and cancer stem cells. Based on this, the authors tested combination of chemotherapy and TGFβ inhibition using a pan-inhibitor of TGFβ (LY2157299) and found that the pan inhibitor of TGFβ enhanced paclitaxel efficacy in a TNBC model (SMU159).

Yet another report provided indirect evidence that TGFβ inhibition may override chemo-resistance in breast cancer (ncbi.nlm.nih.gov/pmc/articles/PMC6457012).

Additionally, Vanpouille-Box et al. showed increased CD8+ T cell infiltration in tumors treated with a combination of radiation therapy and a pan-inhibitor of TGFβ (1 D11) and suggested that TGFβ is a master regulator of radiation therapy-induced antitumor immunity (Cancer Res, 2015). The authors further showed that the triple combination of the pan TGFβ inhibitor, radiation therapy and anti-PD-1 significantly prolonged survival in a preclinical model.

Whilst the relative contribution of the three isoforms of TGFβ was not addressed in these published reports summarized above, the inventors of the present disclosure hypothesize that TGFβ1 is the primary isoform driving the disease phenotype, e.g., resistance to genotoxic therapy. Accordingly, combination and add-on (adjunct) therapies are contemplated herein, in which a TGFβ1-selective inhibitor is used in conjunction with a genotoxic therapy, in the treatment of cancer in a patient, wherein optionally cancer is resistant to a genotoxic therapy. In some embodiments, a TGFβ1-selective inhibitor is used in the treatment of cancer in a patient, wherein the treatment comprises administration of the TGFβ1-selective inhibitor to the patient in an effective amount, wherein the patient is or has been treated with a chemotherapy and/or a radiation therapy. In some embodiments, a genotoxic therapy is used in the treatment of cancer in a patient, wherein the treatment comprises administration of an effective amount of a chemotherapy and/or a radiation therapy to the patient, wherein the patient is or has been treated with a TGFβ1-selective inhibitor. In some embodiments, a TGFβ1-selective inhibitor and a genotoxic therapy (e.g., chemotherapy and/or radiation therapy) are used as a combination therapy in the treatment of cancer in a patient. In preferred embodiments, the TGFβ1-selective inhibitor is Ab6 or a variant thereof.

In certain embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients. The second therapy may diminish or treat at least one symptom(s) associated with the targeted disease. The first and second therapies may exert their biological effects by similar or unrelated mechanisms of action; or either one or both of the first and second therapies may exert their biological effects by a multiplicity of mechanisms of action.

The disclosure provides pharmaceutical compositions and methods for use in, and as, combination therapies for the reduction of TGFβ1 protein activation and the treatment or prevention of diseases or conditions associated with TGFβ1 signaling, as described herein. Accordingly, the methods or the pharmaceutical compositions may further comprise a second therapy. In some embodiments, the methods or pharmaceutical compositions disclosed herein may further comprise a third therapy. In some embodiments, the second therapy and/or the third therapy may be useful in treating or preventing diseases or conditions associated with TGFβ1 signaling. The second therapy and/or the third therapy may diminish or treat at least one symptom(s) associated with the targeted disease. The first, second, and third therapies may exert their biological effects by similar or unrelated mechanisms of action; or either one or both of the first and second therapies may exert their biological effects by a multiplicity of mechanisms of action. In some embodiments, the second therapy and a TGFβ inhibitor disclosed herein (e.g., a TGFβ1-selective inhibitor disclosed herein) are present in a single formulation or in separate formulations contained within in a single package or kit. In some embodiments, the second therapy, the third therapy, and a TGFβ inhibitor disclosed herein (e.g., a TGFβ1-selective inhibitor disclosed herein) are present in a single formulation or in separate formulations contained within in a single package or kit. In some embodiments, the second therapy, and a TGFβ inhibitor disclosed herein (e.g., a TGFβ1-selective inhibitor disclosed herein) are comprised in a single molecule, e.g., in a bispecific antibody or other multispecific construct or, wherein the checkpoint inhibitor is a small molecule, in an antibody-drug conjugate. In some embodiments, the second therapy, the third therapy, and a TGFβ inhibitor disclosed herein (e.g., a TGFβ1-selective inhibitor disclosed herein) are comprised in a single molecule, e.g., in a bispecific antibody or other multispecific construct or, wherein the checkpoint inhibitor is a small molecule, in an antibody-drug conjugate. Examples of engineered constructs with TGFβ inhibitory activities include M7824 (Bintrafusp alfa) and AVID200. M7824 is a bifunctional fusion protein composed of 2 extracellular domains of TGF-pRII (a TGF-p “trap”) fused to a human IgG1 monoclonal antibody against PD-L1. AVID200 is an engineered TGF-p ligand trap comprised of TGF-β receptor ectodomains fused to a human Fc domain.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered concurrently (e.g., simultaneously), separately, or sequentially within described embodiments.

The one or more anti-TGFβantibodies, or antigen binding portions thereof, of the disclosure may be used in conjunction with one or more of additional therapeutic agents. Examples of the additional therapeutic agents which can be used with an anti-TGFβantibody of the disclosure include, but are not limited to: cancer vaccines, engineered immune cell therapies, chemotherapies, radiation therapies (e.g., radiotherapeutic agents), a modulator of a member of the TGFβ superfamily, such as a myostatin inhibitor (e.g., a myostatin inhibitor disclosed in WO2016/073853 and WO2017/049011, the contents of which are hereby incorporated in their entirety), and a GDF11 inhibitor; a VEGF agonist; a VEGF inhibitor (such as bevacizumab); an IGF1 agonist; an FXR agonist; a CCR2 inhibitor; a CCR5 inhibitor; a dual CCR2/CCR5 inhibitor; CCR4 inhibitor, a lysyl oxidase-like-2 inhibitor; an ASK1 inhibitor; an Acetyl-CoA Carboxylase (ACC) inhibitor; a p38 kinase inhibitor; pirfenidone; nintedanib; an M-CSF inhibitor (e.g., M-CSF receptor antagonist and M-CSF neutralizing agents); a MAPK inhibitor (e.g., Erk inhibitor), an immune checkpoint agonist or antagonist; an IL-11 antagonist; and IL-6 antagonist, and the like. Other examples of the additional therapeutic agents which can be used with the TGFβ inhibitors include, but are not limited to, an indoleamine 2,3-dioxygenase (IDO) inhibitor, an arginase inhibitor, a tyrosine kinase inhibitor, Ser/Thr kinase inhibitor, a dual-specific kinase inhibitor. In some embodiments, such an agent may be a PI3K inhibitor, a PKC inhibitor, or a JAK inhibitor. In some embodiments, such an agent may be a TGFβ3-selective inhibitor. In preferred embodiments, the isoform-selective activation inhibitor of TGFβ1 is Ab46, Ab50, a derivative thereof, or an engineered molecule comprising an antigen-binding fragment thereof.

While checkpoint inhibitor (CPI) therapies have transformed the treatment of solid tumors, less than half of cancer patients are eligible for treatment with an approved CPI and of those, <13% respond to CPI therapy (Haslam 2019). Given these data, there remains a significant unmet need across solid tumor indications with approved and unapproved therapies.

Recent data suggest that the effectiveness of immunomodulatory strategies require the presence of a baseline immune response. Tumors lacking a pre-existing immune response or tumors with low numbers of T cells in the tumor core and an enrichment of T cells in the invasive margin or stroma (e.g., in an immune-excluded tumor) have been associated with poor response to CPI (Galon and Bruni 2019. Nat Rev Drug Discov. 18(3): 197-218). The TGFβ pathway has been implicated in mediating primary resistance to CPI therapies, and as such, combination therapy with an anti-latent TGFβ monoclonal antibody may increase efficacy in patients with an inadequate response to CPI monotherapy.

The current disclosure includes use of a TGFβ inhibitor, e.g., Ab6, as a potential anti-cancer therapy alone or in combination with other therapies for the treatment of solid tumors and rare hematological malignancies for which TGFβ signaling dysregulation has been implicated as a mediator of the disease process. In some embodiments, combination therapy comprising a TGFβ inhibitor, e.g., Ab6, and at least one additional agent may be efficacious in patients with advanced solid tumors such as cutaneous melanoma, urothelial carcinoma (UC), non-small cell lung cancer (NSCLC), and head and neck cancer. In some embodiments, combination therapy comprising a TGFβ inhibitor, e.g., Ab6, and at least one additional agent may be efficacious in patients with immune-excluded tumors such as non-small cell lung cancer, melanoma, renal cell carcinoma, triple-negative breast cancer, gastric cancer, microsatellite stable-colorectal cancer, pancreatic cancer, small cell lung cancer, HER2-positive breast cancer, or prostate cancer. In some embodiments, combination therapy comprising a TGFβ inhibitor, e.g., Ab6, and at least one additional agent may be efficacious in patients with immune-infiltrated tumors with an immunosuppressive phenotype. The cancer may be renal cell carcinoma (RCC), especially ccRCC. The cancer may be also be RCC, e.g., ccRCC, where the carcinoma comprises cells that have undergone epithelial-to-mesenchymal transition (EMT).

In some embodiments, the at least one additional agent (e.g., cancer therapy agent) used in a method or composition disclosed herein is a checkpoint inhibitor. In some embodiments, the at least one additional agent is selected from the group consisting of a PD-1 antagonist, a PD-L1 antagonist, a PD-L1 or PD-L2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody (OX40 agonist), an anti-CD27 antibody, an anti-CD70 antibody, an anti-CD47 antibody, an anti-41BB antibody, an anti-PD-1 antibody, an oncolytic virus, and a PARP inhibitor. Exemplary checkpoint inhibitors include, but are not limited to, nivolumab (Opdivo®, anti-PD-1 antibody), pembrolizumab (Keytruda®, anti-PD-1 antibody), cemiplimab (Libtayo®, anti-PD-1 antibody), budigalimab (ABBV-181, anti-PD-1 antibody), BMS-936559 (anti-PD-L1 antibody), atezolizumab (Tecentriq®, anti-PD-L1 antibody), avelumab (Bavencio®, anti-PD-L1 antibody), durvalumab (Imfinzi®, anti-PD-L1 antibody), ipilimumab (Yervoy®, anti-CTLA4 antibody), tremelimumab (anti-CTLA4 antibody), IMP-321 (eftilgimod alpha or “ImmuFact®”, anti-LAG3 large molecule), BMS-986016 (Relatlimab, anti-LAG3 antibody), and lirilumab (anti-KIR2DL-1, -2, -3 antibody). In some embodiments, the TGFβ inhibitors disclosed herein is used in the treatment of cancer in a subject who is a poor responder or non-responder of a checkpoint inhibition therapy, such as those listed herein. In some embodiments, the checkpoint inhibitor and a TGFβ inhibitor (e.g., a TGFβ1-selective inhibitor disclosed herein) are comprised in a single molecule, e.g., in a bispecific antibody or other multispecific construct or, wherein the checkpoint inhibitor is a small molecule, in an antibody-drug conjugate.

In some embodiments, the additional agent is the antibody, GYM329, that specifically binds the latent form of myostatin and inhibits its activation (Muramatsu, H., et al. Sci Rep 11, 2160 (2021)).

In some embodiments, an additional therapy comprises cell therapy, such as CAR-T therapy or CAR-NK therapy.

In some embodiments, the isoform-selective inhibitors of TGFβ1 contemplated herein may be used in conjunction with (e.g., combination therapy, add-on therapy, etc.) a TGFβ3 inhibitor. Such use may further comprise additional therapy, such as therapy intended to treat fibrosis, cancer therapy, e.g., immune checkpoint inhibitor, cancer vaccine, radiation therapy, and/or chemotherapy.

In some embodiments, the isoform-selective inhibitors of TGFβ1 contemplated herein may be used in conjunction with (e.g., combination therapy, add-on therapy, etc.) a selective inhibitor of myostatin (GDF8).

In any of the embodiments contemplated herein for combination therapy, the antibodies disclosed herein, e.g., Ab37, Ab38, Ab39, Ab40, Ab41, Ab43, Ab44, Ab45, Ab46, Ab47, Ab48, Ab49, Ab50, Ab51 and Ab52, may be used. In some embodiments, the preferred TGFβ1 inhibitor is Ab2, Ab46, Ab50, a derivative thereof, or an engineered molecule comprising an antigen-binding fragment thereof.

In some embodiments, the disclosure encompasses use of a TGFβ inhibitor, e.g., Ab6, in combination with at least one checkpoint inhibitor therapy for the treatment of solid tumors and/or hematological malignancies for which TGFβ signaling dysregulation has been implicated as a mediator of the disease process. In certain embodiments, the combination therapy may be administered to patients who are not responsive to checkpoint inhibitor therapy (e.g., anti-PD-1 or anti-PD-L1 therapy). Such patients may include, but are not limited to, those diagnosed with non-small cell lung cancer, urothelial bladder carcinoma, melanoma, triple-negative breast cancer, or other advance solid cancers. In certain embodiments, the combination therapy may comprise a TGFβ inhibitor, e.g., Ab6, and a checkpoint inhibitor therapy (e.g., pembrolizumab). In certain embodiments, the combination therapy may be administered to immunotherapy-naïve patients (e.g., patients who have not previously received a checkpoint inhibitor therapy) diagnosed with a cancer that has received FDA approval for treatment with a checkpoint inhibitor therapy. Such cancer may be gastric cancer (e.g., metastatic gastric cancer), urothelial bladder carcinoma, lung cancer, triple-negative breast cancer, renal cell carcinoma, including clear cell RCC or papillary RCC, cervical cancer, or head and neck squamous cell carcinoma. In certain embodiments, the combination therapy may comprise a TGFβ inhibitor, e.g., Ab6, and a checkpoint inhibitor therapy (e.g., pembrolizumab). Certain embodiments, the combination therapy may further comprise an additional agent, e.g., an additional checkpoint inhibitory and/or another chemotherapeutic agent. In certain embodiments, the combination therapy may be administered to immunotherapy-naïve patients (e.g., patients who have not previously received a checkpoint inhibitor therapy) diagnosed with a cancer that has not received FDA approval for treatment with a checkpoint inhibitor therapy. Such cancer may be a microsatellite-stable colorectal cancer or pancreatic cancer. In certain embodiments, the combination therapy may comprise a TGFβ inhibitor, e.g., Ab6, a checkpoint inhibitor therapy (e.g., pembrolizumab), and at least one chemotherapeutic agent (e.g., axitinib, paclitaxel, cisplatin, and/or 5-fluorouracil). In certain embodiments, the checkpoint inhibitor therapy may be pembrolizumab, nivolumab, and/or atezolizumab. In certain embodiments, the checkpoint inhibitor therapy may be budigalimab (ABBV-181). In certain embodiments, the combination therapy is administered to patients who have cancers characterized as exhibiting an immune-excluded phenotype. In certain embodiments, additional analyses of a patient's cancer may be carried out to further inform treatment, and such analyses may use known cancer-specific markers including microsatellite instability levels, PD-1 and/or PD-L1 expression level, and/or the presence of mutations in known cancer driver genes such as EGFR, ALK, ROS1, BRAF. In certain embodiments, tumor PD-L1 expression may be used as a biomarker of therapeutic response.

In certain embodiments, the TGFβ inhibitor, e.g., Ab6, may be administered in an amount of about 3000 mg, 2400 mg, 2000 mg, 1600 mg, 800 mg, 240 mg, 80 mg, or less. In certain embodiments, the TGFβ inhibitor (e.g., Ab6) may be administered in an amount of about 3000 mg, 2400 mg, 2000 mg, 1600 mg, 800 mg, 240 mg, 80 mg, or less, e.g., at a frequency of once every six weeks, once every four weeks, once every three weeks, once every two weeks, wherein the TGFβ inhibitor (e.g., Ab6) is administered alone or in combination with a checkpoint inhibitor therapy, e.g., an anti-PD-(L)1 therapy. In certain embodiments, the TGFβ inhibitor (e.g., Ab6) may be administered alone at 3000 mg once every three weeks.

In certain embodiments, the TGFβ inhibitor (e.g., Ab6) may be administered alone at 3000 mg once every six weeks. In certain embodiments, the TGFβ inhibitor (e.g., Ab6) may be administered at 3000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every six weeks. In certain embodiments, the TGFβ inhibitor (e.g., Ab6) may be administered at 2400 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every six weeks. In certain embodiments, the TGFβ inhibitor (e.g., Ab6) may be administered at 2000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every six weeks. In certain embodiments, the TGFβ inhibitor (e.g., Ab6) may be administered at 1600 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every six weeks. In certain embodiments, the TGFβ inhibitor (e.g., Ab6) may be administered at 800 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every six weeks. In certain embodiments, the TGFβ inhibitor (e.g., Ab6) may be administered at 240 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every six weeks. In certain embodiments, the TGFβ inhibitor (e.g., Ab6) may be administered at 80 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every six weeks. In certain embodiments, the TGFβ inhibitor (e.g., Ab6) may be administered at an amount of less than 80 mg once every six weeks, wherein the TGFβ inhibitor (e.g., Ab6) is administered in combination with an anti-PD-(L)1 therapy. In some embodiments, the TGFβ inhibitor (e.g., Ab6) may be administered every six weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every four weeks. In certain embodiments, the TGFβ inhibitor (e.g., Ab6) may be administered at 3000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every four weeks. In certain embodiments, the TGFβ inhibitor (e.g., Ab6) may be administered at 2400 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every four weeks. In certain embodiments, the TGFβ inhibitor (e.g., Ab6) may be administered at 2000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every four weeks. In certain embodiments, the TGFβ inhibitor (e.g., Ab6) may be administered at 1600 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every four weeks. In certain embodiments, the TGFβ inhibitor (e.g., Ab6) may be administered at 800 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every four weeks. In certain embodiments, the TGFβ inhibitor (e.g., Ab6) may be administered at 240 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every four weeks. In certain embodiments, the TGFβ inhibitor (e.g., Ab6) may be administered at 80 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every four weeks. In certain embodiments, the TGFβ inhibitor (e.g., Ab6) may be administered at an amount of less than 80 mg once every four weeks, wherein the TGFβ inhibitor (e.g., Ab6) is administered in combination with an anti-PD-(L)1 therapy. In some embodiments, the TGFβ inhibitor (e.g., Ab6) may be administered every four weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every three weeks. In certain embodiments, the TGFβ inhibitor (e.g., Ab6) may be administered at 3000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every three weeks. In certain embodiments, the TGFβ inhibitor (e.g., Ab6) may be administered at 2400 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every three weeks. In certain embodiments, the TGFβ inhibitor (e.g., Ab6) may be administered at 2000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every three weeks. In certain embodiments, the TGFβ inhibitor (e.g., Ab6) may be administered at 1600 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every three weeks. In certain embodiments, the TGFβ inhibitor (e.g., Ab6) may be administered at 800 mg once every three weeks, wherein the TGFβ inhibitor (e.g., Ab6) is administered in combination with an anti-PD-(L) 1 therapy. In certain embodiments, the TGFβ inhibitor (e.g., Ab6) may be administered at 800 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every three weeks. In certain embodiments, the TGFβ inhibitor (e.g., Ab6) may be administered at 240 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every three weeks. In certain embodiments, the TGFβ inhibitor (e.g., Ab6) may be administered at 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every three weeks. In certain embodiments, the TGFβ inhibitor (e.g., Ab6) may be administered at an amount of less than 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of three weeks, wherein the TGFβ inhibitor (e.g., Ab6) is administered in combination with an anti-PD-(L)1 therapy. In some embodiments, the TGFβ inhibitor (e.g., Ab6) may be administered every three weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every two weeks. In certain embodiments, the TGFβ inhibitor (e.g., Ab6) may be administered at 3000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every two weeks. In certain embodiments, the TGFβ inhibitor (e.g., Ab6) may be administered at 2400 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every two weeks. In certain embodiments, the TGFβ inhibitor (e.g., Ab6) may be administered at 2000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every two weeks. In certain embodiments, the TGFβ inhibitor (e.g., Ab6) may be administered at 1600 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every two weeks. In certain embodiments, the TGFβ inhibitor (e.g., Ab6) may be administered at 800 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every two weeks. In certain embodiments, the TGFβ inhibitor (e.g., Ab6) may be administered at 240 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every two weeks. In certain embodiments, the TGFβ inhibitor (e.g., Ab6) may be administered at 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every two weeks. In certain embodiments, the TGFβ inhibitor (e.g., Ab6) may be administered at an amount of less than 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of two weeks, wherein the TGFβ inhibitor (e.g., Ab6) is administered in combination with an anti-PD-(L)1 therapy. In some embodiments, the TGFβ inhibitor (e.g., Ab6) may be administered every two weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) is administered in combination with pembrolizumab to a subject having NSCLC, UC, MEL, esophageal cancer, gastric cancer, HNSCC, HCC, cervical cancer, SCLC, PMBCL, Hodgkin's lymphoma, MSI-H or dMMR cancer, or TMB-H cancer. In certain embodiments, the subject is a non-responder to pembrolizumab. In certain embodiments, the subject has not received pembrolizumab previously. In certain embodiments, the TGFβ inhibitor (e.g., Ab6) is administered in combination with pembrolizumab to a subject having NSCLC who is a non-responder to pembrolizumab treatment. In certain embodiments, the subject having NSCLC has not received pembrolizumab previously. In certain embodiments, the TGFβ inhibitor (e.g., Ab6) is administered in combination with pembrolizumab to a subject having MEL who is a non-responder to pembrolizumab treatment. In certain embodiments, the subject having MEL has not received pembrolizumab previously. In certain embodiments, the TGFβ inhibitor (e.g., Ab6) is administered in combination with pembrolizumab to a subject having UC or mUC who is a non-responder to pembrolizumab treatment. In certain embodiments, the subject having UC or mUC has not received pembrolizumab previously.

In certain embodiments, the TGFβ inhibitor (e.g., Ab6) is administered in combination with budigalimab (ABBV-181) to a subject having NSCLC, UC, MEL, esophageal cancer, gastric cancer, HNSCC, HCC, cervical cancer, SCLC, PMBCL, Hodgkin's lymphoma, MSI-H or dMMR cancer, or TMB-H cancer. In certain embodiments, the subject is a non-responder to budigalimab (ABBV-181). In certain embodiments, the subject has not received budigalimab (ABBV-181) previously. In certain embodiments, the TGFβ inhibitor (e.g., Ab6) is administered in combination with budigalimab (ABBV-181) to a subject having NSCLC who is a non-responder to budigalimab (ABBV-181) treatment. In certain embodiments, the subject having NSCLC has not received budigalimab (ABBV-181) previously. In certain embodiments, the TGFβ inhibitor (e.g., Ab6) is administered in combination with budigalimab (ABBV-181) to a subject having MEL who is a non-responder to budigalimab (ABBV-181) treatment. In certain embodiments, the subject having MEL has not received budigalimab (ABBV-181) previously. In certain embodiments, the TGFβ inhibitor (e.g., Ab6) is administered in combination with budigalimab (ABBV-181) to a subject having UC or mUC who is a non-responder to budigalimab (ABBV-181) treatment. In certain embodiments, the subject having UC or mUC has not received budigalimab (ABBV-181) previously. In certain embodiments, budigalimab is administered once every two weeks. In further embodiments, budigalimab is administered once every two weeks at a dose of 250 mg. In certain embodiments, budigalimab is administered once every three weeks. In further embodiments, budigalimab is administered once every three weeks at a dose of 375 mg. In certain embodiments, budigalimab is administered once every four weeks. In further embodiments, budigalimab is administered once every four weeks at a dose of 500 mg.

In certain embodiments, the TGFβ inhibitor (e.g., Ab6) is administered in combination with nivolumab to a subject having NSCLC, UC, MEL, esophageal cancer, HNSCC, HCC, RCC, Hodgkin's lymphoma, MSI-H or dMMR CRC, or MPM. In certain embodiments, the subject is a non-responder to nivolumab. In certain embodiments, the subject has not received nivolumab previously.

In certain embodiments, the TGFβ inhibitor (e.g., Ab6) is administered in combination with cemiplimab to a subject having BCC or CSCC. In certain embodiments, the subject is a non-responder to cemiplimab. In certain embodiments, the subject has not received cemiplimab previously.

In certain embodiments, the TGFβ inhibitor (e.g., Ab6) is administered in combination with atezolizumab to a subject having NSCLC, MEL, HCC, TNBC, or SCLC. In certain embodiments, the subject is a non-responder to atezolizumab. In certain embodiments, the subject has not received atezolizumab previously.

In certain embodiments, the TGFβ inhibitor (e.g., Ab6) is administered in combination with avelumab to a subject having UC or MCC. In certain embodiments, the subject is a non-responder to avelumab. In certain embodiments, the subject has not received avelumab previously.

In certain embodiments, the TGFβ inhibitor (e.g., Ab6) is administered in combination with durvalumab to a subject having NSCLC or SCLC. In certain embodiments, the subject is a non-responder to durvalumab. In certain embodiments, the subject has not received durvalumab previously.

In certain embodiments, the TGFβ inhibitor (e.g., Ab6) is administered in combination with a checkpoint inhibitor therapy to a subject having a solid tumor for which a checkpoint inhibitor therapy has been approved. In certain embodiments, the subject has a tumor type that has been approved for treatment with a combination of a checkpoint inhibitor therapy and a chemotherapy. In certain embodiments, the subject has a tumor type that typically exhibits immune exclusion in more than 50% of the tumor area (e.g., tumor nests). In certain embodiments, the immune excluded tumor types include triple-negative breast cancer or renal cell carcinoma.

In certain embodiments, the TGFβ inhibitor (e.g., Ab6) is administered in combination with a checkpoint inhibitor therapy to a subject having a solid tumor for which a checkpoint inhibitor monotherapy has been approved. In certain embodiments, the subject has a tumor type that typically exhibits immune exclusion in more than 50% of the tumor area (e.g., tumor nests), such as non-small cell lung cancer, urothelial carcinoma, gastric cancer, and renal cell carcinoma. In certain embodiments, the subject has a tumor type that typically exhibits immune exclusion in less than 50% of the tumor area (e.g., tumor nests), such as small-cell lung cancer or melanoma.

In certain embodiments, the TGFβ inhibitor (e.g., Ab6) is administered in combination with a checkpoint inhibitor therapy to a subject having a solid tumor for which a checkpoint inhibitor has not been approved. In certain embodiments, the subject has a tumor type that typically exhibits immune exclusion in more than 50% of the tumor area (e.g., tumor nests), such as microsatellite stable colorectal cancer, pancreatic cancer, and prostate cancer. Without wishing to be bound by theory, it is possible that tumor types that do not have an approved checkpoint inhibitor therapy respond poorly to checkpoint inhibitor therapy with or without a conventional chemotherapy. Thus, it is contemplated that such tumor types, particularly tumors exhibiting immune exclusion, may have better response to checkpoint inhibitor therapy when combined with a TGFβ inhibitor therapy (e.g., Ab6).

Thus, TGFβ inhibitors (e.g., Ab6) may be used in conjunction (e.g., in combination) with a checkpoint inhibitor therapy for the treatment of cancer in a subject, wherein the cancer comprises an immunosuppressive tumor, and wherein the immunosuppressive tumor is resistant to checkpoint inhibitor therapy. Non-limiting examples of immunosuppressive tumors include acute myeoid leukemia, adrenocortical cancer, brain lower grade glioma, cholangiocarcinoma, colon adenocarcinoma, diffuse large B-cell lymphoma, esophageal carcinoma, glioblastoma multiforme, kidney chromophobe, lung squamous cell carcinoma, mesothelioma, ovarian serous cystadenocarcinoma, pheochromocytoma and paraganglioma, prostate adenocarcinoma, rectum adenocarcinoma, sarcoma, testicular germ cell tumor, thymoma, thyroid carcinoma, uterine carcinosarcoma, uterine corpus endometrioid carcinoma, or uveal melanoma.

In certain embodiments, the disclosure provides a checkpoint inhibitor and a TGFβ1 inhibitor for use in the treatment of cancer in a subject in need thereof, wherein the treatment comprises administration of a checkpoint inhibitor and a TGFβ1 inhibitor in amounts effective to treat cancer, wherein optionally the checkpoint inhibitor is a PD-(L)1 inhibitor, wherein further optionally the PD-(L)1 inhibitor is budigalimab; wherein optionally the TGFβ1 inhibitor is a TGFβ1-selective inhibitor, wherein further optionally the TGFβ1-selective inhibitor is SRK-181 (also referred to as Ab6 herein); and, wherein optionally the cancer comprises a solid tumor of immunosuppressive phenotype.

In some embodiments, the disclosure provides a checkpoint inhibitor for use in the treatment of cancer in a subject in need thereof, wherein the treatment comprises administration of a checkpoint inhibitor to the subject treated with a TGFβ1 inhibitor, in amounts effective to treat cancer, wherein optionally the checkpoint inhibitor is a PD-(L)1 inhibitor, wherein further optionally the PD-(L)1 inhibitor is budigalimab; wherein optionally the TGFβ1 inhibitor is a TGFβ1-selective inhibitor, wherein further optionally the TGFβ1-selective inhibitor is SRK-181 (also referred to as Ab6 herein); and, wherein optionally the cancer comprises a solid tumor of immunosuppressive phenotype.

In some embodiments, the disclosure provides a TGFβ1 inhibitor for use in the treatment of cancer in a subject in need thereof, wherein the treatment comprises administration of a TGFβ1 inhibitor to the subject treated with a checkpoint inhibitor, in amounts effective to treat cancer, wherein optionally the checkpoint inhibitor is a PD-(L)1 inhibitor, wherein further optionally the PD-(L)1 inhibitor is budigalimab; wherein optionally the TGFβ1 inhibitor is a TGFβ1-selective inhibitor, wherein further optionally the TGFβ1-selective inhibitor is SRK-181 (also referred to as Ab6 herein); and, wherein optionally the cancer comprises a solid tumor of immunosuppressive phenotype.

In some embodiments, the at least one additional agent binds a T-cell costimulation molecule, such as inhibitory costimulation molecules and activating costimulation molecules. In some embodiments, the at least one additional agent is selected from the group consisting of an anti-CD40 antibody, an anti-CD38 antibody, an anti-KIR antibody, an anti-CD33 antibody, an anti-CD137 antibody, and an anti-CD74 antibody.

In some embodiments, the at least one additional therapy is radiation. In some embodiments, the at least one additional agent is a radiotherapeutic agent. In some embodiments, the at least one additional agent is a chemotherapeutic agent. In some embodiments, the chemotherapeutic agent is Taxol. In some embodiments, the at least one additional agent is an anti-inflammatory agent. In some embodiments, the at least one additional agent inhibits the process of monocyte/macrophage recruitment and/or tissue infiltration. In some embodiments, the at least one additional agent is an inhibitor of hepatic stellate cell activation. In some embodiments, the at least one additional agent is a chemokine receptor antagonist, e.g., CCR2 antagonists and CCR5 antagonists. In some embodiments, such chemokine receptor antagonist is a dual specific antagonist, such as a CCR2/CCR5 antagonist. In some embodiments, the at least one additional agent to be administered as combination therapy is or comprises a member of the TGFβ superfamily of growth factors or regulators thereof. In some embodiments, such agent is selected from modulators (e.g., inhibitors and activators) of GDF8/myostatin and GDF11. In some embodiments, such agent is an inhibitor of GDF8/myostatin signaling. In some embodiments, such agent is a monoclonal antibody that specifically binds a pro/latent myostatin complex and blocks activation of myostatin. In some embodiments, the monoclonal antibody that specifically binds a pro/latent myostatin complex and blocks activation of myostatin does not bind free, mature myostatin; see, for example, WO 2017/049011.

In some embodiments, an additional therapy comprises cell therapy, such as CAR-T therapy and CAR-NK therapy.

In some embodiments, an additional therapy comprises administering an anti-VEGF therapy, such as a VEGF inhibitor, e.g., bevacizumab. In some embodiments, inhibitors of TGFβ contemplated herein may be used in conjunction with (e.g., combination therapy, add-on therapy, etc.) a VEGF inhibitor (e.g., bevacizumab) for the treatment of solid cancer (e.g., ovarian cancer). In some embodiments, inhibitors of TGFβ contemplated herein may be used in conjunction with (e.g., combination therapy, add-on therapy, etc.) a VEGF inhibitor (e.g., bevacizumab) for the treatment of hematopoietic cancers.

In some embodiments, an additional therapy is a cancer vaccine. Numerous clinical trials that tested peptide-based cancer vaccines have targeted hematological malignancies (cancers of the blood), melanoma (skin cancer), breast cancer, head and neck cancer, gastroesophageal cancer, lung cancer, pancreatic cancer, prostate cancer, ovarian cancer, and colorectal cancers. The antigens included peptides from HER2, telomerase (TERT), survivin (BIRC5), and Wilms' tumor 1 (WT1). Several trials also used “personalized” mixtures of 12-15 distinct peptides. That is, they contain a mixture of peptides from the patient's tumor that the patient exhibits an immune response against. Some trials are targeting solid tumors, glioma, glioblastoma, melanoma, and breast, cervical, ovarian, colorectal, and non-small lung cell cancers and include antigens from MUC1, IDO1 (Indoleamine 2,3-dioxygenase), CTAG1B, and two VEGF receptors, FLT1 and KDR. Notably, the IDO1 vaccine is tested in patients with melanoma in combination with the immune checkpoint inhibitor ipilimumab and the BRAF (gene) inhibitor vemurafenib.

Non-limiting examples of tumor antigens useful as cancer vaccines include: NY-ESO-1, HER2, HPV16 E7 (Papillomaviridae #E7), CEA (Carcinoembryonic antigen), WT1, MART-1, gp100, tyrosinase, URLC10, VEGFR1, VEGFR2, surviving, MUC1 and MUC2.

Activated immune cells primed by such cancer vaccine may, however, be excluded from the TME in part through TGFβ1-dependent mechanisms. To overcome the immunosuppression, use of TGFβ1 inhibitors of the present disclosure may be considered so as to unleash the potential of the vaccine.

Combination therapies contemplated herein may advantageously utilize lower dosages of the administered therapeutic agents, thus avoiding possible toxicities or complications associated with the various monotherapies. In some embodiments, use of an isoform-specific inhibitor of TGFβ1 described herein may render those who are poorly responsive or not responsive to a therapy (e.g., standard of care) more responsive. In some embodiments, use of an isoform-specific inhibitor of TGFβ1 described herein may allow reduced dosage of the therapy (e.g., standard of care) which still produces equivalent clinical efficacy in patients but fewer or lesser degrees of drug-related toxicities or adverse events.

The TGFβ inhibitor may increase the patient's anti-tumor immunity, such as by reducing immunosuppression. For example, the TGFβ inhibitor may reduce immune exclusion (such as by increasing infiltrating tumoral CD8+ cells and/or changing the immune phenotype of the tumor and/or increasing expansion of tumoral CD8+ cells), and/or the TGFβ inhibitor may reduce the suppression that is caused by immunosuppressive cells such as MDSCs and/or Tregs (such as by reducing the level of circulating MDSCs, e.g., circulating g-MDSCs. The TGFβ inhibitor may also enhance the cytotoxic functions of T cells.

In some embodiments, inhibitors of TGFβ contemplated herein may be used in conjunction with (e.g., combination therapy, add-on therapy, etc.) a selective inhibitor of myostatin (GDF8). In some embodiments, the selective inhibitor of myostatin is an inhibitor of pro/latent myostatin activation. See, for example, the antibodies disclosed in WO 2017/049011, such as apitegromab.

### Monotherapy

According to the present disclosure, a TGFβ inhibitor may be used as monotherapy in the treatment of cancer in a subject, wherein optionally the TGFβ inhibitor is a TGFβ1-selective inhibitor. Early clinical data from the DRAGON study indicate that certain cancer types respond to a TGFβ1-selective inhibitor, without checkpoint inhibitor. For example, multiple ovarian cancer patients showed disease stabilization (SD) upon such therapy. At the time of this application, there is no checkpoint inhibitors approved for the treatment of ovarian cancer. It is possible that ovarian cancer and other cancers sharing similar cellular and disease features may have immune excluded or immune desert phenotype. In some embodiments, cancer for which TGFβ inhibitors are administered as monotherapy is a carcinoma. In some embodiments, the cancer is uterine corpus endometrial carcinoma (UCEC), thyroid carcinoma (THCA), testicular germ cell tumors (TGCT), skin cutaneous melanoma (SKCM), prostate adenocarcinoma (PRAD), ovarian serous cystadenocarcinoma (OV), lung squamous cell carcinoma (LUSC), lung adenocarcinoma (LUAD), liver hepatocellular carcinoma (LIHC), kidney renal clear cell carcinoma (KIRC), head and neck squamous cell carcinoma (HNSC), glioblastoma multiforme (GMB), esophageal carcinoma (ESCA), colon adenocarcinoma (COAD), breast invasive carcinoma (BRCA), or bladder urothelial carcinoma (BLCA). In preferred embodiments, the TGFβ inhibitor is a TGFβ1-selective inhibitor, wherein optionally the TGFβ1-selective inhibitor is Ab6. In some embodiments, the subject further receives genotoxic therapy such as chemotherapy and/or radiation therapy. Optionally, the subject may further receive a checkpoint inhibitor therapy.

In some embodiments, the TGFβ inhibitor that is used as a monotherapy, e.g., Ab6, increases the patient's anti-tumor immunity, such as by reducing immunosuppression. For example, the TGFβ inhibitor may reduce immune exclusion (such as by increasing infiltrating tumoral CD8+ cells and/or changing the immune phenotype of the tumor and/or increasing expansion of tumoral CD8+ cells), and/or the TGFβ inhibitor may reduce the suppression that is caused by immunosuppressive cells such as MDSCs and/or Tregs (such as by reducing the level of circulating MDSCs, e.g., circulating g-MDSCs). The TGFβ inhibitor may also enhance the cytotoxic functions of T cells.

### Advantages of TGFβ1 Inhibitors as a Therapeutic

It has been recognized that various diseases involve heterogeneous populations of cells as sources of TGFβ1 that collectively contribute to the pathogenesis and/or progression of the disease. More than one types of TGFβ1-containing complexes (“contexts”) likely coexist within the same disease microenvironment. In particular, such diseases may involve both an ECM (or “matrix”) component of TGFβ1 signaling (e.g., ECM dysregulation) and an immune component of TGFβ1 signaling. In such situations, selectively targeting only a single TGFβ1 context (e.g., TGFβ1 associated with one particular type of presenting molecule) may provide limited relief. Thus, broadly inhibitory TGFβ1 antagonists are desirable for therapeutic use. Previously described inhibitory antibodies that broadly targeted multiple latent complexes of TGFβ1 exhibited skewed binding profiles among the target complexes (see, for example, WO 2018/129329 and WO 2019/075090). The inventors therefore set out to identify more uniformly inhibitory antibodies that selectively inhibit TGFβ1 activation, irrespective of particular presenting molecule linked thereto. It was reasoned that particularly for immune-oncology applications, it is advantageous to potently inhibit both matrix-associated TGFβ1 and immune cell-associated TGFβ1.

In certain situations, so-called context-biased antibodies that still specifically bind to all four antigen complexes but with stronger affinities for matrix-associated complexes over cell-associated complexes may be advantageous. The feature, i.e., differential binding affinities of these antibodies for the ECM complexes relative to immune cell complexes may raise the possibility that such inhibitors may be particularly suited as therapeutics to treat fibrotic conditions, such as organ fibrosis, in which affected patients receive a long-term therapeutic regimen to treat chronic conditions. In these circumstances, it is desirable to minimize unwanted inflammation triggered by immune stimulation.

In various embodiments, context-independent inhibitors of TGFβ1 are used in the treatments and methods disclosed herein to target the pro/latent forms of TGFβ1. More specifically, in one modality, the inhibitor targets ECM-associated TGFβ1 (LTBP1/3-TGFβ1 complexes). In another modality, the inhibitor targets immune cell-associated TGFβ1. This includes GARP-presented TGFβ1, such as GARP-TGFβ1 complexes expressed on Treg cells and LRRC33-TGFβ1 complexes expressed on macrophages and other myeloid/lymphoid cells, as well as certain cancer cells.

Such antibodies may include isoform-specific inhibitors of TGFβ1 that bind and prevent activation (or release) of mature TGFβ1 growth factor from a pro/latent TGFβ1 complex in a context-independent manner, such that the antibodies can inhibit activation (or release) of TGFβ1 associated with multiple types of presenting molecules. In particular, the present disclosure provides antibodies capable of blocking ECM-associated TGFβ1 (LTBP-presented and LTBP3-presented complexes) and cell-associated TGFβ1 (GARP-presented and LRRC33-presented complexes).

Various disease conditions have been suggested to involve dysregulation of TGFβ signaling as a contributing factor. Indeed, the pathogenesis and/or progression of certain human conditions appear to be predominantly driven by or dependent on TGFβ1 activities. In particular, many such diseases and disorders involve both an ECM component and an immune component of TGFβ1 function, suggesting that TGFβ1 activation in multiple contexts (e.g., mediated by more than one type of presenting molecules) is involved. Moreover, it is contemplated that there is crosstalk among TGFβ1-responsive cells. In some cases, interplays between multifaceted activities of the TGFβ1 axis may trigger a cascade of events that lead to disease progression, aggravation, and/or suppression of the host's ability to combat disease. For example, certain disease microenvironments, such as tumor microenvironment (TME) and fibrotic microenvironment (FME), may be associated with TGFβ1 presented by multiple different presenting molecules, e.g., LTBP1-proTGFβ1, LTBP3-proTGFβ1, GARP-proTGFβ1, LRRC33-proTGFβ1, and any combinations thereof. TGFβ1 activities of one context may in turn regulate or influence TGFβ1 activities of another context, raising the possibility that when dysregulated, this may result in exacerbation of disease conditions. Therefore, it is desirable to broadly inhibit across multiple modes of TGFβ1 function (i.e., multiple contexts) while selectively limiting such inhibitory effects to the TGFβ1 isoform. The aim is not to perturb homeostatic TGFβ signaling mediated by the other isoforms, including TGFβ3, which plays an important role in would healing.

Immune components of TGFβ1 activities are largely mediated by cell-associated TGFβ1 (e.g., GARP-proTGFβ1 and LRRC33-proTGFβ1). Both the GARP- and LRRC33-arms of TGFβ1 function are associated with immunosuppressive features that contribute to the progression of many diseases. Thus, TGFβ inhibitors such as the TGFβ1 inhibitors described herein, may be used to inhibit TGFβ1 associated with immunosuppressive cells. The immunosuppressive cells include regulatory T-cells (Tregs), M2 macrophages/tumor-associated macrophages, and MDSCs. The TGFβ inhibitors of the current disclosure may inhibit, reduce, or reverse immunosuppressive phenotype at a disease site such as the tumor microenvironment.

In some embodiments, the TGFβ1 inhibitor inhibits TGFβ1 associated with a cell expressing the GARP-TGFβ1 complex or the LRRC33-TGFβ1 complex, wherein optionally the cell may be a T-cell, a fibroblast, a myofibroblast, a macrophage, a monocyte, a dendritic cell, an antigen presenting cell, a neutrophil, a myeloid-derived suppressor cell (MDSC), a lymphocyte, a mast cell, or a microglial cell. The T-cell may be a regulatory T cell (e.g., immunosuppressive T cell). The neutrophil may be an activated neutrophil. The macrophage may be an activated (e.g., polarized) macrophage, including profibrotic and/or tumor-associated macrophages (TAM), e.g., M2c subtype and M2d subtype macrophages. In some embodiments, macrophages are exposed to tumor-derived factors (e.g., cytokines, growth factors, etc.) which may further induce pro-cancer phenotypes in macrophages. In some embodiments, such tumor-derived factor is CSF-1/M-CSF.

In some embodiments, the cell expressing the GARP-TGFβ1 complex or the LRRC33-TGFβ1 complex is a cancer cell, e.g., circulating cancer cells and tumor cells.

In some embodiments, the antibodies of the present disclosure have greater affinities towards EMC-complexes, e.g., hLTBP1-proTGFβ1 and hLTBP3-proTGFβ1 (KD of <1 nM) over cell-associated complexes, as determined by, for example, solution equilibrium titration. It is envisaged that the EMC-biased antibodies are capable of preferentially targeting and inhibiting EMC-associated TGFβ1 in vivo. Such antibodies may be advantageous for use in the treatment of conditions with ECM dysregulation, such as abnormal remodeling and/or stiffness of the ECM. Typically, the ECM dysregulation may be accompanied by an increased number of myofibroblasts or myofibroblast-like cells in the disease environment, such as tumor microenvironment and fibrotic microenvironment. Many of the abnormal features of the ECM are often manifested in a wide range of pathological conditions, including fibrosis and proliferative disorders are at least in part driven by the TGFβ1 pathway.

The context-biased antibodies with weaker binding to a GARP-associated TGFβ complex (e.g., human GARP-proTGFβ1) may be used in the treatment of a condition where it is undesirable to stimulate the subject's immune response and/or in situations where the subject is expected to benefit from a long-term TGFβ inhibition therapy to manage a chronic condition, such as many types of fibrosis. Rationale for the therapeutic use of a TGFβ1 inhibitor with a weaker binding affinity for GARP-proTGFβ1 is at least threefold:

First, GARP is predominantly expressed on regulatory T cells, which play a crucial role in maintaining immune tolerance to self-antigens and in preventing autoimmune disease. Since Tregs generally suppress, dampen or downregulate induction and proliferation of effector T cells, systemic inhibition of this function may lead to overactive or exaggerated immune responses in the host by disabling the “break” that is normally provided by Treg cells. Thus, the approach taken here (e.g., TGFβ1 inhibition without fully disabling Treg function) is aimed to avoid the risk of eliciting autoimmunity. Furthermore, patients who already have a propensity for developing over-sensitive immune responses or autoimmunity may be particularly at risk of triggering or exacerbating such conditions, without the availability of functional Tregs; and therefore, the inhibitors that at least partially preserve GARP-mediated TGFβ1 function may advantageously minimize such risk.

Second, evidence suggests that an alteration in the Th17/Treg ratio leads to an imbalance in pro-fibrotic Th17 cytokines, which correlate with severity of fibrosis, such as liver fibrosis (see, for example, Shoukry et al., (2017) J Immunol 198 (1 Supplement): 197.12). The present inventors reasoned that disabling perturbation of the GARP arm of TGFβ1 function may directly or indirectly exacerbate fibrotic conditions.

Third, regulatory T cells are indispensable for immune homeostasis and the prevention of autoimmunity. It was reasoned that, particularly for a TGFβ1 inhibition therapy intended for a long-term or chronic administration, it would be desirable to spare at least part of GARP-mediated TGFβ1 and avoid potential side effects stemming from complete perturbation of normal Treg function in maintaining immune homeostasis (reviewed in, for example, Richert-Spuhler and Lund (2015) Prog Mol Biol Transl Sci. 136: 217-243). This strategy is at least in part aimed to preserve normal immune function, which is required, inter alia, for combatting infections.

In some embodiments, a TGFβ inhibitor for use in the methods of the disclosure as described herein is a TGFβ inhibitor described in International Publication No. WO/2021/039945. In some embodiments, a TGFβ inhibitor for use in the methods of treating a fibrotic disorder (e.g., lung fibrosis) is a TGFβ inhibitor described in International Publication No. WO/2021/039945.

### TGFβ Inhibitors Useful for Carrying Out the Invention

TGFβ inhibitors suitable for the therapeutic use and related methods disclosed herein include small molecule (i.e., low molecular weight) antagonists and biologics. Such inhibitors include isoform-selective inhibitors and isoform-non-selective inhibitors. Biologics inhibitors include antibodies, antigen-binding fragments thereof, antibody-based or immunoglobulin-like molecules, as well as other engineered constructs, typically fusion proteins, such as ligand traps. Ligand traps typically include a ligand-binding moiety that is derived from ligand-binding portion or portions of TGFβ receptor(s). Such biologics may be multifunctional constructs, such as bi-functional fusion proteins and bispecific antibodies.

In some embodiments, methods disclosed herein may employ one or more of the following: low molecular weight antagonists of TGFβ receptors, e.g., ALK5 antagonists, such as Galunisertib (LY2157299 monohydrate); monoclonal antibodies (such as neutralizing antibodies) that inhibit all three isoforms (“pan-inhibitor” antibodies) (see, for example, WO 2018/134681); monoclonal antibodies that preferentially inhibit two of the three isoforms (e.g., antibodies against TGFβ1/2 (for example WO 2016/161410) and TGFβ1/3 (for example WO 2006/116002); and engineered molecules (e.g., fusion proteins) such as ligand traps (for example, WO 2018/029367; WO 2018/129331 and WO 2018/158727). In some embodiments, methods disclosed herein may employ one or more of the TGFβ inhibitors disclosed in Batlle and Massague (Immunity, 2019. Apr. 16; 50(4):924-940), the content of which is incorporated herein in its entirety.

In some embodiments, the low molecular weight antagonists of TGFβ receptors may include Vactosertib (TEW-7197, EW-7197), LY3200882, PF-06952229, AZ 12601011, and/or AZ 12799734.

In some embodiments, the neutralizing pan-TGFβ antibody is GC1008, also known as fresolimumab, or a derivative thereof. In some embodiments, such antibody comprises the sequence in accordance with the disclosure of WO/2018/134681. In some embodiments, the pan-TGFβ antibody is SAR439459 or a derivative thereof.

In some embodiments, the TGFβ1/2 antibodies include XPA-42-089 or a derivative thereof.

In some embodiments, the antibody is a neutralizing antibody that specifically binds both TGFβ1 and TGFβ3. In some embodiments such antibody preferentially binds TGFβ1 over TGFβ3. For example, the antibody comprises the sequence in accordance with the disclosure of WO/2006/116002. In some embodiments, the antibody is 21D1.

In some embodiments, the antibody is a neutralizing antibody that specifically binds both TGFβ1 and TGFβ2. In some embodiments, the antibody comprises the sequence in accordance with the disclosure of WO/2016/161410. In some embodiments, the antibody is XOMA-089, or NIS-793.

In some embodiments, the antibody is an activation inhibitor antibody that is selective for TGFβ1. In some embodiments, the antibody comprises the sequence in accordance with the disclosure of WO/2015/015003, WO/2019/075090 or WO/2016/115345.

In some embodiments, the antibody is a neutralizing antibody that is selective for TGFβ1. In some embodiments, the antibody comprises the sequence in accordance with the disclosure of WO/2013/134365 or WO/2018/043734.

In some embodiments, the TGFβ inhibitor is a ligand trap. In some embodiments, the ligand trap comprises the structure in accordance with the disclosure of WO/2018/158727. In some embodiments, the ligand trap comprises the structure in accordance with the disclosure of WO 2018/029367; WO 2018/129331. In some embodiments, the ligand trap is a construct known as CTLA4-TGFbRII. In some embodiments, the ligand trap is a bi-functional fusion protein comprising a checkpoint inhibitor function and a TGFβ inhibitor function. In some embodiments, the bi-functional fusion protein is a construct known as M7824 or PDL1-TGFbRII. In some embodiments, the TGFβ inhibitor is a receptor based TGFβ trap, e.g., AVID200.

In some embodiments, the TGFβ inhibitor is an integrin inhibitor. In some embodiments, the TGFβ inhibitor is an inhibitor of an integrin such as αVβ1, αVβ3, αVβ5, αVβ6, αVβ8, α5β1, αIIbβ3, and/or α8β1. Integrin inhibitors include small molecule inhibitors and antibodies that bind to an integrin and/or inhibit the binding of an integrin to the RGD motif of proTGFβ1 and/or proTGFβ3.

In some embodiments, the TGFβ inhibitor is an inhibitor of latent TGFβ (e.g., latent TGFβ1 or latent TGFβ3). In some embodiments, the TGFβ inhibitor is an inhibitor that binds the RGD motif of proTGFβ1 and/or proTGFβ3.

Isoform-Selective Antibodies of proTGFβ1

Preferably, the therapeutic use and related methods in accordance with the present disclosure are carried out with an isoform-selective inhibitor of TGFβ1, e.g., Ab6 (the sequence of which is as disclosed in PCT/US2019/041373, the contents of which are herein incorporated by reference in its entirety) (see also antibodies that bind proTGFβ1, thereby inhibiting its activation, disclosed in WO2021/039945).

Applicant previously disclosed improved antibodies which embody all or most of the following features: 1) selectivity towards TGFβ1 is maintained to minimize unwanted toxicities associated with pan-inhibition (“isoform-selectivity”) (see, for example, PCT/US2017/021972); 2) exhibit broad binding activities across various biological contexts, or, both matrix-associated and cell-associated categories (“context-independent”) (see, for example, WO 2018/129329); 3) achieve more even or unbiased affinities across multiple antigen complexes (“uniformity”); 4) show strong binding activities for each of the antigen complexes, (“high-affinity”) and have robust inhibitory activities for each context (“potency”) (see, for example, PCT/US2019/041373); and, 5) the preferred mechanism of action is to inhibit the activation step so the inhibitor can target a tissue-tethered, latent TGFβ1 complex, so as to preemptively prevent downstream activation events to achieve durable effects, rather than to directly target soluble/free growth factors (“durability”). As disclosed in PCT/US2019/041373, such TGFβ1 inhibitors are highly potent and highly selective inhibitor of latent TGFβ1 activation. Data presented therein demonstrated, inter alia, that this mechanism of isoform-selective inhibition is sufficient to overcome primary resistance to anti-PD-1 in syngeneic mouse models that closely recapitulate some of the features of primary resistance to CBT found in human cancers. In addition, 6) such inhibitors have an improved safety profile as compared to pan-inhibitors or other isoform-non-selective inhibitors of TGFβ, Together, these efficacy and safety data provide a rationale for exploring the therapeutic use of selective TGFβ1 inhibition to broaden and enhance clinical responses to checkpoint blockade in cancer immunotherapy, as well as to treat a number of additional TGFβ1-related indications.

### General Features of Certain TGFβ1 Inhibitors

Exemplary antibodies that may be used for carrying out the present disclosure are disclosed in WO2020014460, the content of which is incorporated herein by reference in its entirety.

Preferred antibodies and corresponding nucleic acid sequences that encode such antibodies useful for carrying out the present disclosure include one or more of the CDR amino acid sequences shown in Tables 5 and 6. Each set of the H-CDRs (H-CDR1, H-CDR2 and H-CDR3) listed in Table 5 can be combined with the L-CDRs (L-CDR1, L-CDR2 and L-CDR3) provided in Table 6.

Thus, the disclosure provides an isolated antibody or antigen-binding fragment thereof comprising six CDRs (e.g., an H-CDR1, an H-CDR2, an H-CDR3, an L-CDR1, an L-CDR2 and an L-CDR3), wherein, the H-CDR1, H-CDR2 and H-CDR3 are selected from the sets of H-CDRs of the antibodies listed in Table 5, and wherein the L-CDR (comprises QASQDITNYLN (SEQ ID NO: 1078), the L-CDR2 comprises DASNLET (SEQ ID NO: 1079), and the L-CDR3 comprises QQADNHPPWT (SEQ ID NO: 1006), wherein optionally, the H-CDR1 may comprise FTFSSFSMD (SEQ ID NO: 1080); the H-CDR-2 may comprise YISPSADTIYYADSVKG (SEQ ID NO: 1076); and/or, the H-CDR3 may comprise ARGVLDYGDMLMP (SEQ ID NO: 1003). In some embodiments, the antibody or the fragment comprises H-CDR1 having the amino acid sequence FTFSSFSMD (SEQ ID NO: 1080), H-CDR2 having the amino acid sequence YISPSADTIYYADSVKG (SEQ ID NO: 1076), and H-CDR-3 having the amino acid sequence ARGVLDYGDMLMP (SEQ ID NO: 1003); L-CDR1 having the amino acid sequence QASQDITNYLN (SEQ ID NO: 1078), L-CDR2 having the amino acid sequence DASNLET (SEQ ID NO: 1079), and L-CDR3 having the amino acid sequence QQADNHPPWT (SEQ ID NO: 1006).

TABLE 5
Complementary determining regions of the heavy chain of exemplary antibodies,
as determined using the numbering scheme described in Lu et al.

| Ab | H-CDR1 | H-CDR2 | H-CDR3 |
|---|---|---|---|
| Ab4 | FTFSSYSMN | YISSSSSTIYYADSVKG | ARGVLDYGDMLDP |
| | (SEQ ID NO: 1081) | (SEQ ID NO: 1082) | (SEQ ID NO: 1083) |
| Ab5 | FTFSSFSMD | YISPDASTIYYADSVKG | ARGVLDYGDMLDP |
| | (SEQ ID NO: 1080) | (SEQ ID NO: 1084) | (SEQ ID NO: 1083) |
| Ab6 | FTFSSFSMD | YISPSADTIYYADSVKG | ARGVLDYGDMLMP |
| | (SEQ ID NO: 1080) | (SEQ ID NO: 1076) | (SEQ ID NO: 1003) |
| Ab21 | FTFSSFSMD | YISPDASTIYYADSVKG | ARGVLDYGDMLDP |
| | (SEQ ID NO: 1080) | (SEQ ID NO: 1084) | (SEQ ID NO: 1083) |
| Ab22 | FTFGSFSMN | YIHSDASTIYYADSVKG | ARGVLDYGDMLDP |
| | (SEQ ID NO: 1088) | (SEQ ID NO: 1086) | (SEQ ID NO: 1083) |
| Ab23 | FTFSSFSMN | YISPSADTIYYADSVKG | ARGVLDYGDMLDP |
| | (SEQ ID NO: 1087) | (SEQ ID NO: 1076) | (SEQ ID NO: 1083) |
| Ab24 | FTFSSFAMY | YISPDASTIYYADSVKG | ARGVLDYGDMLDP |
| | (SEQ ID NO: 1088) | (SEQ ID NO: 1084) | (SEQ ID NO: 1083) |
| Ab25 | FTFGSFSMD | YISPDASTIYYADSVKG | ARGVLDYGDMLDP |
| | (SEQ ID NO: 1088) | (SEQ ID NO: 1084) | (SEQ ID NO: 1083) |
| Ab26 | FTFSSFSMD | YISPDASTIYYADSVKG | ARGVLDYGDMLDP |
| | (SEQ ID NO: 1080) | (SEQ ID NO: 1084) | (SEQ ID NO: 1083) |
| Ab27 | FTFSFYAMN | YISPDASTIYYADSVKG | ARGVLDYGDMLDP |
| | (SEQ ID NO: 1090) | (SEQ ID NO: 1084) | (SEQ ID NO: 1083) |
| Ab28 | FTFSSFSMD | YISPDASTIYYADSVKG | VRGVLDYGDMLDP |
| | (SEQ ID NO: 1080) | (SEQ ID NO: 1084) | (SEQ ID NO: 1091) |
| Ab29 | FTFSSFAMN | YISPDASTIYYAGSVKG | VRAVLDYGDMLDP |
| | (SEQ ID NO: 1092) | (SEQ ID NO: 1093) | (SEQ ID NO: 1094) |
| Ab30 | FTFSSFSMD | YISPDASTIYYADSVKG | ARGTLDYGDMLDP |
| | (SEQ ID NO: 1080) | (SEQ ID NO: 1084) | (SEQ ID NO: 1095) |
| Ab31 | FTFSSFSMD | YISPDASTIYYADSVKG | ARAVLDYGDMLDP |
| | (SEQ ID NO: 1080) | (SEQ ID NO: 1084) | (SEQ ID NO: 1096) |
| Ab32 | FTFSSFSMN | YISPSADTIYYADSVKG | ARGVWDMGDMLDP |
| | (SEQ ID NO: 1087) | (SEQ ID NO: 1076) | (SEQ ID NO: 1097) |
| Ab33 | FTFSSFSMN | YISPSADTIYYADSVKG | AHGVLDYGDMLDP |
| | (SEQ ID NO: 1087) | (SEQ ID NO: 1076) | (SEQ ID NO: 1098) |
| Ab34 | FTFAFYSMN | YISPDASTIYYADSVKG | ARGVLDYGDMLDP |
| | (SEQ ID NO: 1099) | (SEQ ID NO: 1084) | (SEQ ID NO: 1083) |

TABLE 6
Complementary determining regions of the light
chain of exemplary antibodies, as determined
using the Kabat numbering scheme or the numbering
system of Lu et al.

| L-CDR1 | L-CDR2 | L-CDR3 |
|---|---|---|
| QASQDITNYLN | DASNLET | QQADNHPPWT |
| (SEQ ID NO: | (SEQ ID NO: | (SEQ ID NO: |
| 1078) | 1079) | 1006) |

Determination of CDR sequences within an antibody depends on the particular numbering scheme being employed. Commonly used systems include but are not limited to: Kabat numbering system, IMTG numbering system, Chothia numbering system, and others such as the numbering scheme described by Lu et al., (Lu X et al., Mabs. 2019 January; 11(1):45-57). To illustrate, 6 CDR sequences of Ab6 as defined by four different numbering systems are exemplified below. Any art-recognized CDR numbering systems may be used to define CDR sequences of the antibodies of the present disclosure.

TABLE 7
Six CDRs of an exemplary antibody (Ab6) based on four numbering schemes

| | IMTG numbering | Kabat numbering | Chothia numbering | System of Lu et al. |
|---|---|---|---|---|
| H-CDR1 | GFTFSSFS | SFSMD | GFTFSSF | FTFSSFSMD |
| | (SEQ ID NO: 1001) | (SEQ ID NO: 1075) | (SEQ ID NO: 1168) | (SEQ ID NO: 1080) |
| H-CDR2 | ISPSADTI | YISPSADTIYYADSVKG | SPSADT | YISPSADTIYYADSVKG |
| | (SEQ ID NO: 1002) | (SEQ ID NO: 1076) | (SEQ ID NO: 1169) | (SEQ ID NO: 1076) |
| H-CDR3 | ARGVLDYGDMLMP | GVLDYGDMLMP | GVLDYGDMLMP | ARGVLDYGDMLMP |
| | (SEQ ID NO: 1003) | (SEQ ID NO: 1077) | (SEQ ID NO: 1077) | (SEQ ID NO: 1003) |
| L-CDR1 | QDITNY | QASQDITNYLN | QASQDITNYLN | QASQDITNYLN |
| | (SEQ ID NO: 1004) | (SEQ ID NO: 1078) | (SEQ ID NO: 1078) | (SEQ ID NO: 1078) |
| L-CDR2 | DAS | DASNLET | DASNLET | DASNLET |
| | (SEQ ID NO: 1005) | (SEQ ID NO: 1079) | (SEQ ID NO: 1079) | (SEQ ID NO: 1079) |
| L-CDR3 | QQADNHPPWT | QQADNHPPWT | QQADNHPPWT | QQADNHPPWT |
| | (SEQ ID NO: 1006) | (SEQ ID NO: 1006) | (SEQ ID NO: 1006) | (SEQ ID NO: 1006) |

Amino acid sequences of the heavy chain variable domain and the light chain variable domain of exemplary antibodies of the present disclosure are provided in Table 8. Thus, in some embodiments, the isoform-selective TGFβ1 inhibitor of the present disclosure may be an antibody or an antigen-binding fragment thereof comprising a heavy chain variable domain (V_{H}) and a light chain variable domain (V_{L}), wherein the V_{H} and the V_{L} sequences are selected from any one of the sets of V_{H} and V_{L} sequences listed in Table 8 below.

TABLE 8
Heavy chain variable domains and light chain variable domains of exemplary antibodies

| | Heavy Chain Variable Domain (VH) | Light Chain Variable Domain (VL) |
|---|---|---|
| Ab4 | EVQLVESGGGLVQPGGSLRLSCAASGFTFSS | DIQMTQSPSSLSASVGDRVTITCQASQDITNYLN |
| | YSMNWVRQAPGKGLEWVSYISSSSSTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1100) | (SEQ ID NO: 1008) |
| Ab5 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1101) | (SEQ ID NO: 1008) |
| Ab6 | EVQLVESGGGLVQPGGSLRLSCTASGFTFSS | DIQMTQSPSSLSASVGDRVTITCQASQDITNYLN |
| | FSMDWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVLDYGDMLMPWGQGTLVTVSS | GTKVEIK |
| | (SEQ ID NO: 1007) | (SEQ ID NO: 1008) |
| Ab21 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1102) | (SEQ ID NO: 1008) |
| Ab22 | EVQLVESGGGLVQPGGSLRLSCAASGFTFGS | DIQMTQSPSSLSASVGDRVTITCQASQDITNYLN |
| | FSMNWVRQAPGKGLEWVSYIHSDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1103) | (SEQ ID NO: 1008) |
| Ab23 | EVQLVESGGGLVQPGGSLRLSCAASGFTFSS | DIQMTQSPSSLSASVGDRVTITCQASQDITNYLN |
| | FSMNWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1104) | (SEQ ID NO: 1008) |
| Ab24 | EVQLVESGGGLVQPGRSLRLSCAASGFTFSS | DIQMTQSPSSLSASVGDRVTITCQASQDITNYLN |
| | FAMYWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1105) | (SEQ ID NO: 1008) |
| Ab25 | EVQLVESGGGLVQPGGSLRLSCAASGFTFGS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1106) | (SEQ ID NO: 1008) |
| Ab26 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1107) | (SEQ ID NO: 1008) |
| Ab27 | EVQLVESGGGLVQPGGSLRLSCAASGFTFSF | DIQMTQSPSSLSASVGDRVTITCQASQDITNYLN |
| | YAMNWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 1108) | (SEQ ID NO: 1008) |
| Ab28 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCVRGVLDYGDMLDPWGQGTLVTVSS | GTKVEIK |
| | (SEQ ID NO: 1109) | (SEQ ID NO: 1008) |
| Ab29 | EVQLVESGGGLVQPGRSLRLSCAASGFTFSS | DIQMTQSPSSLSASVGDRVTITCQASQDITNYLN |
| | FAMNWVRQAPGKGLEWVSYISPDASTIYYAG | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCVRAVLDYGDMLDPWGQGTLVTVSS | GTKVEIK |
| | (SEQ ID NO: 1110) | (SEQ ID NO: 1008) |
| Ab30 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGTLDYGDMLDPWGQGTLVTVSS | GTKVEIK |
| | (SEQ ID NO: 1111) | (SEQ ID NO: 1008) |
| Ab31 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARAVLDYGDMLDPWGQGTLVTVSS | GTKVEIK |
| | (SEQ ID NO: 1112) | (SEQ ID NO: 1008) |
| Ab32 | EVQLVESGGGLVQPGGSLRLSCAASGFTFSS | DIQMTQSPSSLSASVGDRVTITCQASQDITNYLN |
| | FSMNWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVWDMGDMLDPWGQGTLVTVSS | GTKVEIK |
| | (SEQ ID NO: 1113) | (SEQ ID NO: 1008) |
| Ab33 | EVQLVESGGGLVQPGGSLRLSCAASGFTFSS | DIQMTQSPSSLSASVGDRVTITCQASQDITNYLN |
| | FSMNWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCAHGVLDYGDMLDPWGQGTLVTVSS | GTKVEIK |
| | (SEQ ID NO: 1114) | (SEQ ID NO: 1008) |
| Ab34 | EVQLVESGGGLVQPGGSLRLSCAASGFTFAF | DIQMTQSPSSLSASVGDRVTITCQASQDITNYLN |
| | YSMNWVRQAPGKGLEWVSYISPDAST IYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVT VSS | GTKVEIK |
| | (SEQ ID NO: 1115) | (SEQ ID NO: 1008) |

In some embodiments, an antibody or an antigen-binding fragment thereof is disclosed that comprises a heavy chain variable domain and a light chain variable domain, wherein, the heavy chain variable domain has at least 90% (e.g., at least 90%, 91%, 92%, 93%, 94%, 95%, 96%, 97%, 98% 99% and 100%) sequence identity with any one of the sequences selected from the group consisting of: Ab4, Ab5, Ab6, Ab21, Ab22, Ab23, Ab24, Ab25, Ab26, Ab27, Ab28, Ab29, Ab30, Ab31, Ab32, Ab33, and Ab34; and, wherein the light chain variable domain has at least 90% identity with any one of the sequences selected from Ab4, Ab5, Ab6, Ab21, Ab22, Ab23, Ab24, Ab25, Ab26, Ab27, Ab28, Ab29, Ab30, Ab31, Ab32, Ab33, and Ab34, wherein, optionally, the heavy chain variable domain may optionally have at least 95% sequence identity, and/or, the light chain variable domain may have at least 95% (e.g., at least 95%, 96%, 97%, 98% 99% and 100%) sequence identity. In some embodiments, the heavy chain variable domain of the antibody or the fragment has at least 90% sequence identity with SEQ ID NO: 1007, and wherein optionally, the light chain variable domain of the antibody or the fragment has at least 90% sequence identity with SEQ ID NO: 1008. In some embodiments, the heavy chain variable domain of the antibody or the fragment has at least 95% sequence identity with SEQ ID NO: 1007, and wherein optionally, the light chain variable domain of the antibody or the fragment has at least 95% sequence identity with SEQ ID NO: 1008. In some embodiments, the heavy chain variable domain of the antibody or the fragment has at least 98% sequence identity with SEQ ID NO: 1007, and wherein optionally, the light chain variable domain of the antibody or the fragment has at least 98% sequence identity with SEQ ID NO: 1008. In some embodiments, the heavy chain variable domain of the antibody or the fragment has 100% sequence identity with SEQ ID NO: 1007, and wherein optionally, the light chain variable domain of the antibody or the fragment has 100% sequence identity with SEQ ID NO: 1008.

In various embodiments, an antibody or an antigen-binding fragment thereof disclosed herein comprises 6 CDRs from, or the full sequences of, the heavy and light chain variable domains of SEQ ID Nos: 1007 and 1008, respectively. In some embodiments, the antibody or an antigen-binding fragment thereof comprises heavy and light chain variable domain sequences with at least 90% sequence identity (e.g., at least 95% identity) to SEQ ID Nos: 1007 and 1008, respectively. For instance, the antibody or an antigen-binding fragment thereof may comprise a set of 6 respective H- and L-CDRs selected from those set out in Tables 13 and 14 above. In some certain embodiments, the antibody or antigen-binding fragment thereof comprises a set of 6 respective H- and L-CDRs as set out in Table 7 (e.g., using the system of Lu et al.).

Alternatively, or in addition, the antibody or an antigen-binding fragment thereof used in the context of the present disclosure may comprise heavy and light chain variable domains with at least 90% sequence identity (e.g., at least 95% identity) to SEQ ID Nos: 1007 and 1008, respectively, and specifically binds a proTGFβ1 complex at (i) a first binding region comprising at least a portion of Latency Lasso (SEQ ID NO: 1126); and ii) a second binding region comprising at least a portion of Finger-1 (SEQ ID NO: 1124); characterized in that when bound to the proTGFβ1 complex in a solution, the antibody or the fragment protects the binding regions from solvent exposure as determined by hydrogen-deuterium exchange mass spectrometry (HDX-MS). The first binding region may comprise PGPLPEAV (SEQ ID NO: 1134) or a portion thereof and the second binding region may comprise RKDLGWKW (SEQ ID NO: 1143) or a portion thereof. As used herein, protection of the binding region refers to protein-protein interactions, such as antibody-antigen binding, the degree by which a protein (e.g., a region of a protein containing an epitope) is exposed to a solvent as assessed by an HDX-MS-based assay of protein-protein interactions. Protection of binding may be determined by the level of proton exchange occurring at a binding site, which is inversely correlates with the degree of binding/interaction. Therefore, when an antibody described herein binds to a region of an antigen, the binding region is “protected” from being exposed to the solvent because the protein-protein interaction precludes the binding region from being accessible by the surrounding solvent. The protected region is thus indicative of a site of interaction. The antibody or the fragment may further bind the proTGFβ1 complex at one or more of the following binding regions or a portion thereof: LVKRKRIEA (SEQ ID NO: 1132); LASPPSQGEVPPGPL (SEQ ID NO: 1126); LALYNSTR (SEQ ID NO: 1135); REAVPEPVL (SEQ ID NO: 1136); YQKYSNNSWR (SEQ ID NO: 1137); RKDLGWKWIHE (SEQ ID NO: 1144); HEPKGYHANF (SEQ ID NO: 1145); LGPCPYIWS (SEQ ID NO: 1139); ALEPLPIV (SEQ ID NO: 1140); and, VGRKPKVEQL (SEQ ID NO: 1141).

In some embodiments, the antibody or antigen-binding fragments may further be characterized in that it cross-blocks (cross-competes) for binding to TGFβ1 (e.g., to pro- and/or latent-TGFβ1) with an antibody having the heavy chain variable domain of SEQ ID NO: 1007, and the light chain variable domain of SEQ ID NO: 1008. In some embodiments, the antibody that cross-blocks or cross-competes comprises heavy and light chain variable domains that are at least about 90% (e.g., 95% or 99%) identical to those of SEQ ID Nos 1007 and 1008, respectively.

In some embodiments, the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a heavy chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 1007, and a light chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 1008. In some embodiments, the antibody or antigen binding portion thereof, comprises a heavy chain variable domain amino acid sequence encoded by the nucleic acid sequence set forth in SEQ ID NO: 1007, and a light chain variable domain amino acid sequence encoded by the nucleic acid sequence set forth in SEQ ID NO: 1008.

In some examples, any of the antibodies of the disclosure that specifically bind to a GARP-TGFβ1 complex, a LTBP1-TGFβ1 complex, a LTBP3-TGFβ1 complex, and/or a LRRC33-TGFβ1 complex include any antibody (including antigen binding portions thereof) having one or more CDR (e.g., CDRH or CDRL) sequences substantially similar to CDRH1, CDRH2, CDRH3, CDRL1, CDRL2, and/or CDRL3. For example, the antibodies may include one or more CDR sequences as shown in Table 5 containing up to 5, 4, 3, 2, or 1 amino acid residue variations as compared to the corresponding CDR region in any one of SEQ ID Nos: 1003, 1006, 1076, 1078, 1079, 1080, 1081, 1082, 1083 1084, 1085, 1086, 1087, 1088, 1089, 1090, 1091, 1092, 1093, 1094, 1095, 1096, 1097, 1098, and 1099. In some embodiments, one or more of the six CDR sequences contain up to three (3) amino acid changes as compared to the sequences provided in Table 5. Such antibody variants comprising up to 3 amino acid changes per CDR are encompassed by the present disclosure. In some embodiments, such variant antibodies are generated by the process of optimization, such as affinity maturation. The complete amino acid sequences for the heavy chain variable region and light chain variable region of the antibodies listed in Table 8 (e.g., Ab6), as well as nucleic acid sequences encoding the heavy chain variable region and light chain variable region of certain antibodies are provided below:

Ab6 - Heavy chain variable region amino acid sequence
(SEQ ID NO: 1007)
EVQLVESGGGLVQPGGSLRLSCTASGFTFSSFSMDWVRQAPGKGLEWVSYISPSADTIYYADSVKGRFTISRDN
AKNTLYLQMNSLRAEDTAVYYCARGVLDYGDMLMPWGQGTLVTVSS
Ab6 - Light chain variable region amino acid sequence
(SEQ ID NO: 1008)
DIQMTQSPSSLSASVGDRVTITCQASQDITNYLNWYQQKPGKAPKLLIYDASNLETGVPSRFSGSGSGTDFTFTIS
SLQPEDIATYYCQQADNHPPWTFGGGTKVEIK
Ab6 - Heavy chain amino acid sequence
(SEQ ID NO: 1009)
EVQLVESGGGLVQPGGSLRLSCTASGFTFSSFSMDWVRQAPGKGLEWVSYISPSADTIYYADSVKGRFTISRDN
AKNTLYLQMNSLRAEDTAVYYCARGVLDYGDMLMPWGQGTLVTVSSASTKGPSVFPLAPCSRSTSESTAALGC
LVKDYFPEPVTVSWNSGALTSGVHTFPAVLQSSGLYSLSSVVTVPSSSLGTKTYTCNVDHKPSNTKVDKRVESK
YGPPCPPCPAPEFLGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEALHNHYTQKSLSL
SLG
Ab6 - Heavy chain nucleic acid sequence
(SEQ ID NO: 1010)
GAGGTGCAGCTGGTGGAGTCTGGGGGAGGCTTGGTACAGCCTGGGGGGTCCCTGAGACTCTCCTGTACAG
CCTCTGGATTCACCTTCAGTAGCTTCAGCATGGACTGGGTCCGCCAGGCTCCAGGGAAGGGGCTGGAGTG
GGTTTCATACATTAGTCCCAGTGCAGACACCATATACTACGCAGACTCTGTGAAGGGCCGATTCACCATCTC
CAGAGACAATGCCAAGAACACGCTGTATCTGCAAATGAACAGCCTGAGAGCCGAGGACACGGCGGTGTACT
ACTGCGCCAGAGGGGTGCTCGACTACGGAGACATGTTAATGCCATGGGGCCAGGGAACCCTGGTCACCGT
CTCCTCAGCGTCGACCAAGGGCCCTTCCGTGTTCCCTCTGGCCCCTTGCTCCCGGTCCACCTCCGAGTCCA
CCGCCGCTCTGGGCTGTCTGGTGAAGGACTACTTCCCTGAGCCTGTGACCGTGAGCTGGAACTCTGGCGC
CCTGACCTCCGGCGTGCACACCTTCCCTGCCGTGCTGCAGTCCTCCGGCCTGTACTCCCTGTCCTCCGTGG
TGACCGTGCCTTCCTCCTCCCTGGGCACCAAGACCTACACCTGCAACGTGGACCACAAGCCTTCCAACACC
AAGGTGGACAAGCGGGTGGAGTCCAAGTACGGCCCTCCTTGCCCTCCCTGCCCTGCCCCTGAGTTCCTGG
GCGGACCCTCCGTGTTCCTGTTCCCTCCTAAGCCTAAGGACACCCTGATGATCTCCCGGACCCCTGAGGTG
ACCTGCGTGGTGGTGGACGTGTCCCAGGAAGATCCTGAGGTCCAGTTCAATTGGTACGTGGATGGCGTGG
AGGTGCACAACGCCAAGACCAAGCCTCGGGAGGAACAGTTCAACTCCACCTACCGGGTGGTGTCTGTGCT
GACCGTGCTGCACCAGGACTGGCTGAACGGCAAGGAATACAAGTGCAAGGTCAGCAACAAGGGCCTGCCC
TCCTCCATCGAGAAAACCATCTCCAAGGCCAAGGGCCAGCCTCGCGAGCCTCAGGTGTACACCCTGCCTCC
TAGCCAGGAAGAGATGACCAAGAATCAGGTGTCCCTGACATGCCTGGTGAAGGGCTTCTACCCTTCCGATA
TCGCCGTGGAGTGGGAGAGCAACGGCCAGCCAGAGAACAACTACAAGACCACCCCTCCTGTGCTGGACTC
CGACGGCTCCTTCTTCCTGTACTCCAGGCTGACCGTGGACAAGTCCCGGTGGCAGGAAGGCAACGTCTTTT
CCTGCTCCGTGATGCACGAGGCCCTGCACAACCACTACACCCAGAAGTCCCTGTCCCTGTCTCTGGGC
Ab6 - Light chain amino acid sequence
(SEQ ID NO: 1011)
DIQMTQSPSSLSASVGDRVTITCQASQDITNYLNWYQQKPGKAPKLLIYDASNLETGVPSRFSGSGSGTDF
TFTISSLQPEDIATYYCQQADNHPPWTFGGGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNRGEC
Ab6 - Light chain nucleic acid sequence (human kappa)
(SEQ ID NO: 1012)
GACATCCAGATGACCCAGTCTCCATCCTCCCTGTCTGCATCTGTAGGAGACAGAGTCACCATCACTTGCCAG
GCGAGTCAGGACATTACCAACTATTTAAATTGGTATCAGCAGAAACCAGGGAAAGCCCCTAAGCTCCTGATC
TACGATGCATCCAATTTGGAAACAGGGGTCCCATCAAGGTTCAGTGGAAGTGGATCTGGGACAGATTTTACT
TTCACCATCAGCAGCCTGCAGCCTGAAGATATTGCAACATATTACTGTCAGCAGGCCGACAATCACCCTCCT
TGGACTTTTGGCGGAGGGACCAAGGTTGAGATCAAACGTACGGTGGCTGCACCATCTGTCTTCATCTTCCC
GCCATCTGATGAGCAGTTGAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGAGAAACACAA
AGTCTACGCCTGCGAAGTCACCCATCAGGGCCTGAGCTCGCCCGTCACAAAGAGCTTCAACAGGGGAGAG
TGT

In some embodiments, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al., J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In any of the antibodies or antigen-binding fragments described herein, one or more conservative mutations can be introduced into the CDRs or framework sequences at positions where the residues are not likely to be involved in an antibody-antigen interaction. In some embodiments, such conservative mutation(s) can be introduced into the CDRs or framework sequences at position(s) where the residues are not likely to be involved in interacting with a GARP-TGFβ1 complex, a LTBP1-TGFβ1 complex, a LTBP3-TGFβ1 complex, and a LRRC33 -TGFβ1 complex as determined based on the crystal structure. In some embodiments, likely interface (e.g., residues involved in an antigen-antibody interaction) may be deduced from known structural information on another antigen sharing structural similarities.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, NewYork, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like (CPPCP (SEQ ID NO: 1043)) hinge sequence. Accordingly, any of the antibodies may include a stabilizing ‘Adair’ mutation or the amino acid sequence CPPCP (SEQ ID NO: 1043).

Isoform-specific, context-independent inhibitors of TGFβ1 of the present disclosure may optionally comprise antibody constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

Additionally or alternatively, such antibodies may or may not include the framework region of the antibodies of SEQ ID Nos: 1100, 1101, 1102, 1103, 1104, 1105, 1106, 1107, 1108, 1109, 1110, 1111, 1112, 1113, 1114, 1115, and 1008. In some embodiments, antibodies that specifically bind to a GARP-TGFβ1 complex, a LTBP1-TGFβ1 complex, a LTBP3-TGFβ1 complex, and a LRRC33-TGFβ1 complex are murine antibodies and include murine framework region sequences.

In some embodiments, such antibodies bind to a GARP-TGFβ1 complex, a LTBP1-TGFβ1 complex, a LTBP3-TGFβ1 complex, and a LRRC33-TGFβ1 complex with relatively high affinity, e.g., with a KD less than 10⁻⁹ M, 10⁻¹⁰ M, 10⁻¹¹ M or lower. For example, such antibodies may bind a GARP-TGFβ1 complex, a LTBP1-TGFβ1 complex, a LTBP3-TGFβ1 complex, and/or a LRRC33-TGFβ1 complex with an affinity between 5 pM and 1 nM, e.g., between 10 pM and 1 nM, e.g., between 10 pM and 500 pM. The disclosure also includes antibodies or antigen binding fragments that compete with any of the antibodies described herein for binding to a GARP-TGFβ1 complex, a LTBP1-TGFβ1 complex, a LTBP3-TGFβ1 complex, and/or a LRRC33-TGFβ1 complex and that have a KD value of 1 nM or lower (e.g., 1 nM or lower, 500 pM or lower, 100 pM or lower). The affinity and binding kinetics of the antibodies that specifically bind to a GARP-TGFβ1 complex, a LTBP1-TGFβ1 complex, a LTBP3-TGFβ1 complex, and/or a LRRC33-TGFβ1 complex can be tested using any suitable method including but not limited to biosensor-based technology (e.g., OCTET® or Biacore®) and solution equilibrium titration-based technology (e.g., MSD-SET). In some embodiments, affinity and binding kinetics are measured by SPR, such as Biacore systems. In preferred embodiments, such antibodies dissociate from each of the aforementioned large latent complex with an OFF rate of 10e−4 or less.

In some embodiments, inhibitors of cell-associated TGFβ1 (e.g., GARP-presented TGFβ1 and LRRC33-presented TGFβ1) according to the disclosure include antibodies or fragments thereof that specifically bind such complex (e.g., GARP-pro/latent TGFβ1 and LRRC33-pro/latent TGFβ1) and trigger internalization of the complex. This mode of action causes removal or depletion of the inactive TGFβ1 complexes (e.g., GARP-proTGFβ1 and LRRC33-proTGFβ1) from the cell surface (e.g., Treg, macrophages, etc.), hence reducing TGFβ1 available for activation. In some embodiments, such antibodies or fragments thereof bind the target complex in a pH-dependent manner such that binding occurs at a neutral or physiological pH, but the antibody dissociates from its antigen at an acidic pH; or, dissociation rates are higher at acidic pH than at neutral pH. Such antibodies or fragments thereof may function as recycling antibodies.

Antibodies Competing with the Preferred Antibodies of TGFβ1

Aspects of the disclosure relate to antibodies that compete or cross-compete with any of the antibodies provided herein. The term “compete”, as used herein with regard to an antibody, means that a first antibody binds to an epitope (e.g., an epitope of a GARP-proTGFβ1 complex, a LTBP1-proTGFβ1 complex, a LTBP3-proTGFβ1 complex, and a LRRC33-proTGFβ1 complex) in a manner sufficiently similar to or overlapping with the binding of a second antibody, such that the result of binding of the first antibody with its epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its epitope or ligand, whether to the same, greater, or lesser extent, the antibodies are said to “cross-compete” with each other for binding of their respective epitope(s). Both competing and cross-competing antibodies are within the scope of this disclosure. Regardless of the mechanism by which such competition or cross-competition occurs (e.g., steric hindrance, conformational change, or binding to a common epitope, or portion thereof), the skilled artisan would appreciate that such competing and/or cross-competing antibodies are encompassed and can be useful for the methods and/or compositions provided herein. The term “cross-blocking” may be used interchangeably.

Two different monoclonal antibodies (or antigen-binding fragments) that bind the same antigen may be able to simultaneously bind to the antigen if the binding sites are sufficiently further apart in the three-dimensional space such that each binding does not interfere with the other binding. By contrast, two different monoclonal antibodies may have binding regions of an antigen that are the same or overlapping, in which case, binding of the first antibody may prevent the second antibody from being able to bind the antigen, or vice versa. In the latter case, the two antibodies are said to “cross-block” with each other with respect to the same antigen.

Antibody “binning” experiments are useful for classifying multiple antibodies that are made against the same antigen into various “bins” based on the relative cross-blocking activities. Each “bin” therefore represents a discrete binding region(s) of the antigen. Antibodies in the same bin by definition cross-block each other. Binning can be examined by standard in vitro binding assays, such as Biacore or Octet®, using standard test conditions, e.g., according to the manufacturer's instructions (e.g., binding assayed at room temperature, ~20-25° C.).

Aspects of the disclosure relate to antibodies that compete or cross-compete with any of the specific antibodies, or antigen binding portions thereof, as provided herein. In some embodiments, an antibody, or antigen binding portion thereof, binds at or near the same epitope as any of the antibodies provided herein. In some embodiments, an antibody, or antigen binding portion thereof, binds near an epitope if it binds within 15 or fewer amino acid residues of the epitope. In some embodiments, any of the antibody, or antigen binding portion thereof, as provided herein, binds within 1, 2, 3, 4, 5, 6, 7, 8, 9, 10, 11, 12, 13, 14 or 15 amino acid residues of an epitope that is bound by any of the antibodies provided herein.

In another embodiment, provided herein is an antibody, or antigen binding portion thereof, competes or cross-competes for binding to any of the antigens provided herein (e.g., a GARP-TGFβ1 complex, a LTBP1-TGFβ1 complex, a LTBP3-TGFβ1 complex, and/or a LRRC33-TGFβ1 complex) with an equilibrium dissociation constant, K_{D}, between the antibody and the protein of less than 10⁻⁸ M. In other embodiments, an antibody competes or cross-competes for binding to any of the antigens provided herein with a K_{D} in a range from 10⁻¹² M to 10⁻⁹ M. In some embodiments, provided herein is an anti-TGFβ1 antibody, or antigen binding portion thereof that competes for binding with an antibody, or antigen binding portion thereof, described herein. In some embodiments, provided herein is an anti-TGFβ1 antibody, or antigen binding portion thereof, that binds to the same epitope as an antibody, or antigen binding portion thereof, described herein.

Any of the antibodies provided herein can be characterized using any suitable methods. For example, one method is to identify the epitope to which the antigen binds, or “epitope mapping.” There are many suitable methods for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). In some embodiments, the epitope is a TGFβ1 epitope that is only available for binding by the antibody, or antigen binding portion thereof, described herein, when the TGFβ1 is in a GARP-proTGFβ1 complex, a LTBP1-proTGFβ1 complex, a LTBP3-proTGFβ1 complex, or a LRRC33-proTGFβ1 complex. Peptides of varying lengths (e.g., at least 4-6 amino acids long) can be isolated or synthesized (e.g., recombinantly) and used for binding assays with an antibody. In another example, the epitope to which the antibody binds can be determined in a systematic screen by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the GARP-proTGFβ1 complex, a LTBP1-proTGFβ1 complex, a LTBP3-proTGFβ1 complex, and/or a proLRRC33-TGFβ1 complex have been replaced (swapped) with sequences from a closely related, but antigenically distinct protein, such as another member of the TGFβ protein family (e.g., GDF11).

Alternatively, competition assays can be performed using other antibodies known to bind to the same antigen to determine whether an antibody binds to the same epitope as the other antibodies. Competition assays are well known to those of skill in the art.

In some embodiments, a pharmaceutical composition may be made by a process comprising a step of: selecting an antibody or antigen-binding fragment thereof, which cross-competes with an antibody having a heavy chain variable domain of SEQ ID NO: 1007 and a light chain variable domain of SEQ ID NO: 1008 for binding to TGFβ1 (e.g., to pro-TGFβ1 and/or latent TGFβ1).

In some embodiments, a pharmaceutical composition may be made by the process comprising a step of: selecting an antibody or antigen-binding fragment thereof, which cross-competes with the antibody selected from the group consisting of Ab4, Ab5, Ab6, Ab21, Ab22, Ab23, Ab24, Ab25, Ab26, Ab27, Ab28, Ab29, Ab30, Ab31, Ab32, Ab33, Ab34, and Ab46; and, formulating into a pharmaceutical composition.

Preferably, the antibody selected by the process is a high-affinity binder characterized in that the antibody or the antigen-binding fragment is capable of binding to each of human LLCs (e.g., hLTBP1-proTGFβ1, hLTBP3-proTGFβ1, hGARP-proTGFβ1 and hLRRC33-proTGFβ1) with a K_{D} of ≤1 nM, as measured by solution equilibrium titration. Such cross-competing antibodies may be used in the treatment of TGFβ1-related indications a subject in accordance with the present disclosure.

### Various Modifications and Variations of Antibodies

Non-limiting variations, modifications, and features of any of the antibodies or antigen-binding fragments thereof encompassed by the present disclosure are briefly discussed below. Embodiments of related analytical methods are also provided.

Naturally-occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human antibody light chains are typically classified as kappa and lambda light chains. Heavy chains are typically classified as mu, delta, gamma, alpha, or epsilon, and define the isotype of the antibody. An antibody can be of any type (e.g., IgM, IgD, IgG, IgA, IgY, and IgE) and class (e.g., IgG₁, IgG₂, IgG₃, IgG₄, IgM₁, IgM₂, IgA₁, and IgA₂). Within full-length light and heavy chains, typically, the variable and constant regions are joined by a “J” region of about 12 or more amino acids, with the heavy chain also including a “D” region of about 10 more amino acids (see, e.g., Fundamental Immunology, Ch. 7 (Paul, W., ed., 2^{nd} ed. Raven Press, N.Y. (1989)) (incorporated by reference in its entirety)). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk (1987) J. Mol. Biol. 196: 901-917; Chothia et al., (1989) Nature 342: 878-883. The CDRs of a light chain can also be referred to as CDR-L1, CDR-L2, and CDR-L3, and the CDRs of a heavy chain can also be referred to as CDR-H1, CDR-H2, and CDR-H3. In some embodiments, an antibody can comprise a small number of amino acid deletions from the carboxy end of the heavy chain(s). In some embodiments, an antibody comprises a heavy chain having 1-5 amino acid deletions in the carboxy end of the heavy chain. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In some embodiments, various methods of analysis can be employed to identify or approximate the CDR regions. Examples of such methods include, but are not limited to, the Kabat definition, the Chothia definition, the AbM definition, the definition described by Lu et al (see above), and the contact definition.

An “affinity matured” antibody is an antibody with one or more alterations in one or more CDRs thereof, which result in an improvement in the affinity of the antibody for antigen compared to a parent antibody, which does not possess those alteration(s). Exemplary affinity matured antibodies will have nanomolar or even picomolar affinities (e.g., K_{D} of ~10⁻⁹ M-10⁻¹² M range) for the target antigen. Affinity matured antibodies are produced by procedures known in the art. Marks et al., (1992) Bio/Technology 10: 779-783 describes affinity maturation by VH and VL domain shuffling. Random mutagenesis of CDR and/or framework residues is described by Barbas, et al., (1994) Proc Nat. Acad. Sci. USA 91: 3809-3813; Schier et al., (1995) Gene 169: 147-155; Yelton et al., (1995) J. Immunol. 155: 1994-2004; Jackson et al., (1995) J. Immunol. 154(7): 3310-9; and Hawkins et al., (1992) J. Mol. Biol. 226: 889-896; and selective mutation at selective mutagenesis positions, contact or hypermutation positions with an activity enhancing amino acid residue is described in U.S. Pat. No. 6,914,128. Typically, a parent antibody and its affinity-matured progeny (e.g., derivatives) retain the same binding region within an antigen, although certain interactions at the molecular level may be altered due to amino acid residue alternation(s) introduced by affinity maturation.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “chimeric antibody” refers to antibodies, which comprise heavy and light chain variable region sequences from one species and constant region sequences from another species, such as antibodies having murine heavy and light chain variable regions linked to human constant regions.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

In some embodiments, the antibody or antigen-binding fragment thereof comprises a heavy chain framework region 1 (H-FR1) having the following amino acid sequence with optionally 1, 2 or 3 amino acid changes: EVQLVESGGGLVQPGGSLRLSCAASG (SEQ ID NO: 1147). For example, the Gly residue at position 16 may be replaced with an Arg (R); and/or, the Ala residue at position 23 may be replaced with a Thr (T).

In some embodiments, the antibody or antigen-binding fragment thereof comprises a heavy chain framework region 2 (H-FR2) having the following amino acid sequence with optionally 1, 2 or 3 amino acid changes: WVRQAPGKGLEWVS (SEQ ID NO: 1148).

(SEQ ID NO: 1148)
WVRQAPGKGLEWVS.

In some embodiments, the antibody or antigen-binding fragment thereof comprises a heavy chain framework region 3 (H-FR3) having the following amino acid sequence with optionally 1, 2 or 3 amino acid changes: RFTISRDNAKNSLYLQMNSLRAEDTAVYYC (SEQ ID NO: 1149). For example, the Ser residue at position 12 may be replaced with a Thr (T).

In some embodiments, the antibody or antigen-binding fragment thereof comprises a heavy chain framework region 4 (H-FR4) having the following amino acid sequence with optionally 1, 2 or 3 amino acid changes:

(SEQ ID NO: 1150)
WGQGTLVTVSS.

In some embodiments, the antibody or antigen-binding fragment thereof comprises a light chain framework region 1 (L-FR1) having the following amino acid sequence with optionally 1, 2 or 3 amino acid changes:

(SEQ ID NO: 1151)
DIQMTQSPSSLSASVGDRVTITC.

In some embodiments, the antibody or antigen-binding fragment thereof comprises a light chain framework region 2 (L-FR2) having the following amino acid sequence with optionally 1, 2 or 3 amino acid changes:

(SEQ ID NO: 1152)
WYQQKPGKAPKLLIY.

In some embodiments, the antibody or antigen-binding fragment thereof comprises a light chain framework region 3 (L-FR3) having the following amino acid sequence with optionally 1, 2 or 3 amino acid changes:

(SEQ ID NO: 1153)
GVPSRFSGSGSGTDFTFTISSLQPEDIATYYC.

In some embodiments, the antibody or antigen-binding fragment thereof comprises a light chain framework region 4 (L-FR4) having the following amino acid sequence with optionally 1, 2 or 3 amino acid changes:

(SEQ ID NO: 1154)
FGGGTKVEIK.

In some embodiments, the antibody, or antigen binding portion thereof, comprises a heavy chain immunoglobulin constant domain of a human IgM constant domain, a human IgG constant domain, a human IgG1 constant domain, a human IgG2 constant domain, a human IgG2A constant domain, a human IgG2B constant domain, a human IgG2 constant domain, a human IgG3 constant domain, a human IgG3 constant domain, a human IgG4 constant domain, a human IgA constant domain, a human IgA1 constant domain, a human IgA2 constant domain, a human IgD constant domain, or a human IgE constant domain. In some embodiments, the antibody, or antigen binding portion thereof, comprises a heavy chain immunoglobulin constant domain of a human IgG1 constant domain or a human IgG4 constant domain. In some embodiments, the antibody, or antigen binding portion thereof, comprises a heavy chain immunoglobulin constant domain of a human IgG4 constant domain. In some embodiments, the antibody, or antigen binding portion thereof, comprises a heavy chain immunoglobulin constant domain of a human IgG4 constant domain having a backbone substitution of Ser to Pro that produces an IgG1-like hinge and permits formation of inter-chain disulfide bonds.

In some embodiments, the antibody or antigen binding portion thereof, further comprises a light chain immunoglobulin constant domain comprising a human Ig lambda constant domain or a human Ig kappa constant domain.

In some embodiments, the antibody is an IgG having four polypeptide chains which are two heavy chains and two light chains.

In some embodiments, wherein the antibody is a humanized antibody, a diabody, or a chimeric antibody. In some embodiments, the antibody is a humanized antibody. In some embodiments, the antibody is a human antibody. In some embodiments, the antibody comprises a framework having a human germline amino acid sequence.

In some embodiments, the antigen binding portion is a Fab fragment, a F(ab′)2 fragment, a scFab fragment, or an scFv fragment.

In some embodiments, the antibody contains one or more amino acid modifications in the Fc region. In some embodiments, modifications in the Fc region may provide altered properties, such as altered half life in circulation, e.g., by altering affinity for Fc receptors such as FcRn (Front Immunol. 2019 Jun. 7; 10:1296). In some embodiments, modifications to the Fc region of the antibody provides increased FcγR binding. In some embodiments, modifications in the Fc region provide improved antibody effector function. In some embodiments, modifications in the Fc region provide increased in vivo half-life for the antibody. In some embodiments, the half life of the antibody may be increased by increasing its affinity for binding to FcRn at low pH (e.g., pH<6.5). In some embodiments, such Fc modifications may include Met252Tyr, Ser254Thr, and Thr256Glu substitutions (see, e.g., U.S. Pat. No. 7,083,784).

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al., (2002) Crit. Rev. Immunol. 22(3): 183-200; Marchalonis et al., (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen (e.g., target protein) when a binding protein specifically binds to the antigen. In an embodiment, the neutralizing binding protein binds to the antigen/target, e.g., cytokine, kinase, growth factor, cell surface protein, soluble protein, phosphatase, or receptor ligand, and reduces its biologically activity by at least about 20%, 40%, 60%, 80%, 85%, 90%, 95%, 96%, 97%, 98%, 99% or more. In some embodiments, a neutralizing antibody to a growth factor specifically binds a mature, soluble growth factor that has been released from a latent complex, thereby preventing its ability to bind its receptor to elicit downstream signaling. In some embodiments, the mature growth factor is TGFβ1 or TGFβ3. The term “binding protein” as used herein includes any polypeptide that specifically binds to an antigen (e.g., TGFβ1), including, but not limited to, an antibody, or antigen binding portions thereof, and a bispecific or multispecific construct that comprises an antigen binding region (e.g., a region capable of binding TGFβ1) and a region capable of binding one or more additional antigens or additional epitopes on a single antigen. Examples include a DVD-IgTM, a TVD-Ig, a RAb-Ig, a bispecific antibody, and a dual specific antibody. A binding protein may also comprise an antibody-drug conjugate, e.g., wherein a second agent (e.g., a small molecule checkpoint inhibitor) is linked to an antibody or antigen-binding fragment thereof capable of binding TGFβ1 (e.g., capable of binding pro- and/or latent-TGFβ1)

The term “monoclonal antibody” or “mAb” when used in a context of a composition comprising the same may refer to an antibody preparation obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35: 425-445; Gavilondo, J. V. and Larrick, J. W. (2002) BioTechniques 29: 128-145; Hoogenboom, H. and Chames, P. (2000) Immunol. Today 21: 371-378, incorporated herein by reference), antibodies isolated from an animal (e.g., a mouse) that is transgenic for human immunoglobulin genes (see, Taylor, L. D. et al., (1992) Nucl. Acids Res. 20: 6287-6295; Kellermann, S-A. and Green, L. L. (2002) Cur. Opin. in Biotechnol. 13: 593-597; Little, M. et al., (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “Dual Variable Domain Immunoglobulin” or “DVD-IgTM” and the like include binding proteins comprising a paired heavy chain DVD polypeptide and a light chain DVD polypeptide with each paired heavy and light chain providing two antigen binding sites. Each binding site includes a total of 6 CDRs involved in antigen binding per antigen binding site. A DVD-IgTM is typically has two arms bound to each other at least in part by dimerization of the CH3 domains, with each arm of the DVD being bispecific, providing an immunoglobulin with four binding sites. DVD-IgTM are provided in US Patent Publication Nos. 2010/0260668 and 2009/0304693, each of which are incorporated herein by reference including sequence listings.

As used herein, “Triple Variable Domain Immunoglobulin” or “TVD-Ig” and the like are binding proteins comprising a paired heavy chain TVD binding protein polypeptide and a light chain TVD binding protein polypeptide with each paired heavy and light chain providing three antigen binding sites. Each binding site includes a total of 6 CDRs involved in antigen binding per antigen binding site. A TVD binding protein may have two arms bound to each other at least in part by dimerization of the CH3 domains, with each arm of the TVD binding protein being trispecific, providing a binding protein with six binding sites.

As used herein, “Receptor-Antibody Immunoglobulin” or “RAb-Ig” and the like are binding proteins comprising a heavy chain RAb polypeptide, and a light chain RAb polypeptide, which together form three antigen binding sites in total. One antigen binding site is formed by the pairing of the heavy and light antibody variable domains present in each of the heavy chain RAb polypeptide and the light chain RAb polypeptide to form a single binding site with a total of 6 CDRs providing a first antigen binding site. Each the heavy chain RAb polypeptide and the light chain RAb polypeptide include a receptor sequence that independently binds a ligand providing the second and third “antigen” binding sites. a RAb-Ig typically has two arms bound to each other at least in part by dimerization of the CH3 domains, with each arm of the RAb-Ig being trispecific, providing an immunoglobulin with six binding sites. RAb-Igs are described in US Patent Application Publication No. 2002/0127231, the entire contents of which including sequence listings are incorporated herein by reference).

In various embodiments, the present disclosure provides, in part, novel antibodies and antigen-binding fragments that may be used alone, linked to one or more additional agents (e.g., as ADCs), or as part of a larger macromolecule (e.g., a bispecific antibody, dual-specific antibody, or as a multispecific antibody, or as part of a construct further comprising a ligand trap, e.g., in combination with a TGFB ligand trap such as M7824 (Merck) and AVID200 (Forbius)), or as part of a bifunctional or multifunctional engineered construct (e.g., fusion proteins and ligand traps) and may be administered as part of pharmaceutical compositions or combination therapies.

The term “bispecific antibody,” as used herein, and as differentiated from a “bispecific half-Ig binding protein” or “bispecific (half-Ig) binding protein”, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al., (1985) Nature 314(6012): 628-631), or by knob-into-hole or similar approaches, which introduce mutations in the Fc region that do not inhibit CH3-CH3 dimerization (see Holliger, P. et al., (1993) Proc. Natl. Acad. Sci USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen it binds to. For example, a bispecific antibody comprising two binding arms directed toward TGFβ1 and PD-1 may be used to combine a TGFβ1 inhibitor (Ab6 or Ab6-derived binding moiety) and a checkpoint inhibitor (e.g., an anti-PD1 antibody or moiety). Such a bispecific antibody may be used as an exemplary form of treatment for patients selected to receive a TGFβ1 inhibitor and checkpoint inhibitor combination therapy.

The term “dual-specific antibody,” as used herein, and as differentiated from a bispecific half-Ig binding protein or bispecific binding protein, refers to full-length antibodies that can bind two different antigens (or epitopes) in each of its two binding arms (a pair of HC/LC) (see PCT Publication No. WO 02/02773). Accordingly, a dual-specific binding protein has two identical antigen binding arms, with identical specificity and identical CDR sequences, and is bivalent for each antigen to which it binds.

The term “multispecific antibody” refers to an antibody or antigen binding fragment that displays binding specificity for two or more epitopes, where each binding site differs and recognizes a different epitope (on the same or different antigens). A bispecific antibody is an exemplary type of multispecific antibody. Higher order multispecifics (i.e., antibodies exhibiting more than two specificities) include but are not limited to trispecific antibodies in TriMAb, triple body, and tribody formats. For exemplary types of multispecific antibodies and/or methods of generating the same, see, e.g., Castoldi et al., Protein Eng Des Sel 2012; 25:551-9; Schubert et al., MAbs 2011; 3:21-30; Kugler et al., Br J Haematol 2010; 150:574-86; Schoonjans et al., J Immunol 2000; 165:7050-7; and Egan et al., MAbs 2017; 9(1):68-84, which are all incorporated herein by reference for such types and methods.

The term “Kon,” as used herein, is intended to refer to the on rate constant for association of a binding protein (e.g., an antibody) to the antigen to form the, e.g., antibody/antigen complex as is known in the art. The “Kon” also is known by the terms “association rate constant,” or “ka,” as used interchangeably herein. This value indicating the binding rate of an antibody to its target antigen or the rate of complex formation between an antibody and antigen also is shown by the equation: Antibody (“Ab”)+Antigen (“Ag”)→Ab-Ag.

The term “Koff,” as used herein, is intended to refer to the off rate constant for dissociation of a binding protein (e.g., an antibody) from the, e.g., antibody/antigen complex as is known in the art. The “Koff” also is known by the terms “dissociation rate constant” or “kd” as used interchangeably herein. This value indicates the dissociation rate of an antibody from its target antigen or separation of Ab-Ag complex over time into free antibody and antigen as shown by the equation: Ab+Ag←-Ab-Ag.

The terms “equilibrium dissociation constant” or “K_{D},” as used interchangeably herein, refer to the value obtained in a titration measurement at equilibrium, or by dividing the dissociation rate constant (koff) by the association rate constant (kon). The association rate constant, the dissociation rate constant, and the equilibrium dissociation constant are used to represent the binding affinity of a binding protein, e.g., antibody, to an antigen. Methods for determining association and dissociation rate constants are well known in the art. Using fluorescence-based techniques offers high sensitivity and the ability to examine samples in physiological buffers at equilibrium. Other experimental approaches and instruments, such as a Biacore® (biomolecular interaction analysis) assay, can be used (e.g., instrument available from Biacore International AB, a GE Healthcare company, Uppsala, Sweden). Additionally, a KinExA® (Kinetic Exclusion Assay) assay, available from Sapidyne Instruments (Boise, Idaho), can also be used.

The terms “crystal” and “crystallized” as used herein, refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach 2nd ed., pp. 201-16, Oxford University Press, New York, New York, (1999). The term “linker” is used to denote polypeptides comprising two or more amino acid residues joined by peptide bonds and are used to link one or more antigen binding portions. Such linker polypeptides are well known in the art (see, e.g., Holliger, P. et al., (1993) Proc. Natl. Acad. Sci. USA 90: 6444-6448; Poljak, R. J. et al., (1994) Structure 2:1121-1123). Exemplary linkers include, but are not limited to, ASTKGPSVFPLAP (SEQ ID NO: 1044), ASTKGP (SEQ ID NO: 1045); TVAAPSVFIFPP (SEQ ID NO: 1046); TVAAP (SEQ ID NO: 1047); AKTTPKLEEGEFSEAR (SEQ ID NO: 1048); AKTTPKLEEGEFSEARV (SEQ ID NO: 1049); AKTTPKLGG (SEQ ID NO: 1050); SAKTTPKLGG (SEQ ID NO: 1051); SAKTTP (SEQ ID NO: 1052); RADAAP (SEQ ID NO: 1053); RADAAPTVS (SEQ ID NO: 1054); RADAAAAGGPGS (SEQ ID NO: 1055); RADAAAA (G4S)4 (SEQ ID NO: 1056); SAKTTPKLEEGEFSEARV (SEQ ID NO: 1057); ADAAP (SEQ ID NO: 1058); ADAAPTVSIFPP (SEQ ID NO: 1059); QPKAAP (SEQ ID NO: 1060); QPKAAPSVTLFPP (SEQ ID NO: 1061); AKTTPP (SEQ ID NO: 1062); AKTTPPSVTPLAP (SEQ ID NO: 1063); AKTTAP (SEQ ID NO: 1064); AKTTAPSVYPLAP (SEQ ID NO: 7576); GGGGSGGGGSGGGGS (SEQ ID NO: 1066); GENKVEYAPALMALS (SEQ ID NO: 1067); GPAKELTPLKEAKVS (SEQ ID NO: 1068); GHEAAAVMQVQYPAS (SEQ ID NO: 1069); TVAAPSVFIFPPTVAAPSVFIFPP (SEQ ID NO: 1070); and ASTKGPSVFPLAPASTKGPSVFPLAP (SEQ ID NO: 1071). “Label” and “detectable label” or “detectable moiety” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Thus, the term “labeled binding protein” as used herein, refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ¹⁸F, ¹¹C, ¹³N, ¹⁵O, ⁶⁸Ga, ¹⁸F, ⁸⁹Zr, ³H, ¹⁴C, ³⁵S ⁹⁰Y, ⁹⁹Tc, ¹¹¹In, ¹²⁵I, ¹³¹I, ¹⁷⁷Lu, ¹⁶⁶Ho, and ¹⁵³Sm); chromogens; fluorescent labels (e.g., FITC, rhodamine, and lanthanide phosphors); enzymatic labels (e.g., horseradish peroxidase, luciferase, and alkaline phosphatase); chemiluminescent markers; biotinyl groups; predetermined polypeptide epitopes recognized by a secondary reporter (e.g., leucine zipper pair sequences, binding sites for secondary antibodies, metal binding domains, and epitope tags); and magnetic agents, such as gadolinium chelates. Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of “detectably labeled” is intended to encompass the latter type of detectable labeling.

In some embodiments, the binding affinity of an antibody, or antigen binding portion thereof, to an antigen (e.g., protein complex), such as presenting molecule-proTGFβ1 complexes, is determined using BLI (e.g., an Octet® assay). A BLI (e.g., Octet®) assay is an assay that determines one or more a kinetic parameters indicative of binding between an antibody and antigen. In some embodiments, an Octet® system (FortéBio®, Menlo Park, CA) is used to determine the binding affinity of an antibody, or antigen binding portion thereof, to presenting molecule-proTGFβ1 complexes. For example, binding affinities of antibodies may be determined using the ForteBio Octet® QKe dip and read label free assay system utilizing bio-layer interferometry. In some embodiments, antigens are immobilized to biosensors (e.g., streptavidin-coated biosensors) and the antibodies and complexes (e.g., biotinylated presenting molecule-proTGFβ1 complexes) are presented in solution at high concentration (50 pg/mL) to measure binding interactions. In some embodiments, the binding affinity of an antibody, or antigen binding portion thereof, to a presenting molecule-proTGFβ1 complex is determined using the protocol outlined herein, e.g., in Table 46.

### Antigen Complexes and Components Thereof

The novel antibodies of the present disclosure specifically bind each of the four known human large latency complexes (e.g., hLTBP1-proTGFβ1, hLTBP3-proTGFβ1, hGARP-proTGFβ1 and hLRRC33-proTGFβ1), selectively inhibits TGFβ1 activation.

Some antibodies, and antigen-binding fragments thereof, of the present disclosure specifically bind each of the four known human large latency complexes (e.g., hLTBP1-proTGFβ1, hLTBP3-proTGFβ1, hGARP-proTGFβ1 and hLRRC33-proTGFβ1), with low dissociation rates, selectively inhibits TGFβ1 activation, and, satisfy safety criteria discussed herein. Screening (e.g., identification and selection) of such antibodies involves the use of suitable antigen complexes, which are typically recombinantly produced.

Screening (e.g., identification and selection) of such antibodies involves the use of suitable antigen complexes, which are typically recombinantly produced. Useful protein components that may comprise such antigen complexes are provided, including TGFβ isoforms and related polypeptides, fragments and variants, presenting molecules (e.g., LTBPs, GARP, LRRC33) and related polypeptides, fragments and variants. These components may be expressed, purified, and allowed to form a protein complex (such as large latent complexes), which can be used in the process of antibody screening. The screening may include positive selection, in which desirable binders are selected from a pool or library of binders and non-binders, and negative selection, in which undesirable binders are removed from the pool. Typically, at least one matrix-associated complex (e.g., LTBP1-proTGFβ1 and/or LTBP1-proTGFβ1) and at least one cell-associated complex (e.g., GARP-proTGFβ1 and/or LRRC33-proTGFβ1) are included for positive screening to ensure that binders being selected have affinities for both such biological contexts.

In some embodiments, the TGFβ1 comprises a naturally occurring mammalian amino acid sequence. In some embodiment, the TGFβ1 comprises a naturally occurring human amino acid sequence. In some embodiments, the TGFβ1 comprises a human, a monkey, a rat or a mouse amino acid sequence. In some embodiments, an antibody, or antigen binding portion thereof, described herein does not specifically bind to TGFβ2. In some embodiments, an antibody, or antigen binding portion thereof, described herein does not specifically bind to TGFβ3. In some embodiments, an antibody, or antigen binding portion thereof, described herein does not specifically bind to TGFβ2 or TGFβ3. In some embodiments, an antibody, or antigen binding portion thereof, described herein specifically binds to a TGFβ1 comprising the amino acid sequence set forth in SEQ ID NO: 1023. The amino acid sequences of TGFβ2, and TGFβ3 amino acid sequence are set forth in SEQ ID NOs: 1027 and 1021, respectively. In some embodiments, an antibody, or antigen binding portion thereof, described herein specifically binds to a TGFβ1 comprising a non-naturally-occurring amino acid sequence (otherwise referred to herein as a non-naturally-occurring TGFβ1). For example, a non-naturally-occurring TGFβ1 may comprise one or more recombinantly generated mutations relative to a naturally-occurring TGFβ1 amino acid sequence. In some embodiments, a TGFβ1, TGFβ2, or TGFβ3 amino acid sequence comprises the amino acid sequence as set forth in SEQ ID NOs: 1013-1024, as shown in Table 9. In some embodiments, a TGFβ1, TGFβ2, or TGFβ3 amino acid sequence comprises the amino acid sequence as set forth in SEQ ID NOs: 1025-1032, as shown in Table 10.

TGFβ1 (prodomain + growth factor domain)
(SEQ ID NO: 1013)
LSTCKTIDMELVKRKRIEAIRGQILSKLRLASPPSQGEVPPGPLPEAVLALYNSTRDRVAGESAEPEPEPEADYYA
KEVTRVLMVETHNEIYDKFKQSTHSIYMFFNTSELREAVPEPVLLSRAELRLLRLKLKVEQHVELYQKYSNNSWRY
LSNRLLAPSDSPEWLSFDVTGVVRQWLSRGGEIEGFRLSAHCSCDSRDNTLQVDINGFTTGRRGDLATIHGMNR
PFLLLMATPLERAQHLQSSRHRRALDTNYCFSSTEKNCCVRQLYIDFRKDLGWKWIHEPKGYHANFCLGPCPYI
WSLDTQYSKVLALYNQHNPGASAAPCCVPQALEPLPIVYYVGRKPKVEQLSNMIVRSCKCS
TGFβ2 (prodomain + growth factor domain)
(SEQ ID NO: 1017)
SLSTCSTLDMDQFMRKRIEAIRGQILSKLKLTSPPEDYPEPEEVPPEVISIYNSTRDLLQEKASRRAAACERERSDE
EYYAKEVYKIDMPPFFPSENAIPPTFYRPYFRIVRFDVSAMEKNASNLVKAEFRVFRLQNPKARVPEQRIELYQILK
SKDLTSPTQRYIDSKVVKTRAEGEWLSFDVTDAVHEWLHHKDRNLGFKISLHCPCCTFVPSNNYIIPNKSEELEAR
FAGIDGTSTYTSGDQKTIKSTRKKNSGKTPHLLLMLLPSYRLESQQTNRRKKRALDAAYCFRNVQDNCCLRPLYI
DFKRDLGWKWIHEPKGYNANFCAGACPYLWSSDTQHSRVLSLYNTINPEASASPCCVSQDLEPLTILYYIGKTPKI
EQLSNMIVKSCKCS
TGFβ3 (prodomain + growth factor domain)
(SEQ ID NO: 1021)
SLSLSTCTTLDFGHIKKKRVEAIRGQILSKLRLTSPPEPTVMTHVPYQVLALYNSTRELLEEMHGEREEGCTQENT
ESEYYAKEIHKFDMIQGLAEHNELAVCPKGITSKVFRFNVSSVEKNRTNLFRAEFRVLRVPNPSSKRNEQRIELFQI
LRPDEHIAKQRYIGGKNLPTRGTAEWLSFDVTDTVREWLLRRESNLGLEISIHCPCHTFQPNGDILENIHEVMEIKF
KGVDNEDDHGRGDLGRLKKQKDHHNPHLILMMIPPHRLDNPGQGGQRKKRALDTNYCFRNLEENCCVRPLYID
FRQDLGWKWVHEPKGYYANFCSGPCPYLRSADTTHSTVLGLYNTLNPEASASPCCVPQDLEPLTILYYVGRTPK
VEQLSNMVVKSCKCS

TABLE 9
Exemplary TGFβ1, TGFβ2, and TGFβ3 amino acid sequences

| | | SEQ ID |
|---|---|---|
| Protein | Sequence | NO |
| proTGFβ1 | LSTCKTIDMELVKRKRIEAIRGQILSKLRLASPPSQGEVPPGPLPEAVLAL | 1013 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHRRALDTNYCFSSTEKNCCVRQLYIDFRKDLGWKWIHEPKGYHANFC | |
| | LGPCPYIWSLDTQYSKVLALYNQHNPGASAAPCCVPQALEPLPIVYYVG | |
| | RKPKVEQLSNMIVRSCKCS | |
| proTGFβ1 C4S | LSTSKTIDMELVKRKRIEAIRGQILSKLRLASPPSQGEVPPGPLPEAVLAL | 1014 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHRRALDTNYCFSSTEKNCCVRQLYIDFRKDLGWKWIHEPKGYHANFC | |
| | LGPCPYIWSLDTQYSKVLALYNQHNPGASAAPCCVPQALEPLPIVYYVG | |
| | RKPKVEQLSNMIVRSCKCS | |
| proTGFβ1 D2G | LSTCKTIDMELVKRKRIEAIRGQILSKLRLASPPSQGEVPPGPLPEAVLAL | 1015 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHGALDTNYCFSSTEKNCCVRQLYIDFRKDLGWKWIHEPKGYHANFCL | |
| | GPCPYIWSLDTQYSKVLALYNQHNPGASAAPCCVPQALEPLPIVYYVGR | |
| | KPKVEQLSNMIVRSCKCS | |
| proTGFβ1 C4S D2G | LSTSKTIDMELVKRKRIEAIRGQILSKLRLASPPSQGEVPPGPLPEAVLAL | 1016 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHGALDTNYCFSSTEKNCCVRQLYIDFRKDLGWKWIHEPKGYHANFCL | |
| | GPCPYIWSLDTQYSKVLALYNQHNPGASAAPCCVPQALEPLPIVYYVGR | |
| | KPKVEQLSNMIVRSCKCS | |
| proTGFβ2 | SLSTCSTLDMDQFMRKRIEAIRGQILSKLKLTSPPEDYPEPEEVPPEVISI | 1017 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKKRALDAAYCFRNVQDN | |
| | CCLRPLYIDFKRDLGWKWIHEPKGYNANFCAGACPYLWSSDTQHSRVL | |
| | SLYNTINPEASASPCCVSQDLEPLTILYYIGKTPKIEQLSNMIVKSCKCS | |
| proTGFβ2 C5S | SLSTSSTLDMDQFMRKRIEAIRGQILSKLKLTSPPEDYPEPEEVPPEVISI | 1018 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKKRALDAAYCFRNVQDN | |
| | CCLRPLYIDFKRDLGWKWIHEPKGYNANFCAGACPYLWSSDTQHSRVL | |
| | SLYNTINPEASASPCCVSQDLEPLTILYYIGKTPKIEQLSNMIVKSCKCS | |
| proTGFβ2 C5S D2G | SLSTSSTLDMDQFMRKRIEAIRGQILSKLKLTSPPEDYPEPEEVPPEVISI | 1019 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKGALDAAYCFRNVQDNC | |
| | CLRPLYIDFKRDLGWKWIHEPKGYNANFCAGACPYLWSSDTQHSRVLS | |
| | LYNTINPEASASPCCVSQDLEPLTILYYIGKTPKIEQLSNMIVKSCKCS | |
| proTGFβ2 D2G | SLSTCSTLDMDQFMRKRIEAIRGQILSKLKLTSPPEDYPEPEEVPPEVISI | 1020 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKGALDAAYCFRNVQDNC | |
| | CLRPLYIDFKRDLGWKWIHEPKGYNANFCAGACPYLWSSDTQHSRVLS | |
| | LYNTINPEASASPCCVSQDLEPLTILYYIGKTPKIEQLSNMIVKSCKCS | |
| proTGFβ3 | SLSLSTCTTLDFGHIKKKRVEAIRGQILSKLRLTSPPEPTVMTHVPYQVLA | 1021 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKKRALDTNYCFRNLEENCC | |
| | VRPLYIDFRQDLGWKWVHEPKGYYANFCSGPCPYLRSADTTHSTVLGL | |
| | YNTLNPEASASPCCVPQDLEPLTILYYVGRTPKVEQLSNMVVKSCKCS | |
| proTGFβ3 C7S | SLSLSTSTTLDFGHIKKKRVEAIRGQILSKLRLTSPPEPTVMTHVPYQVLA | 1022 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKKRALDTNYCFRNLEENCC | |
| | VRPLYIDFRQDLGWKWVHEPKGYYANFCSGPCPYLRSADTTHSTVLGL | |
| | YNTLNPEASASPCCVPQDLEPLTILYYVGRTPKVEQLSNMVVKSCKCS | |
| proTGFβ3 | SLSLSTSTTLDFGHIKKKRVEAIRGQILSKLRLTSPPEPTVMTHVPYQVLA | 1023 |
| C7S D2G | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKGALDTNYCFRNLEENCCV | |
| | RPLYIDFRQDLGWKWVHEPKGYYANFCSGPCPYLRSADTTHSTVLGLY | |
| | NTLNPEASASPCCVPQDLEPLTILYYVGRTPKVEQLSNMVVKSCKCS | |
| proTGFβ3 D2G | SLSLSTCTTLDFGHIKKKRVEAIRGQILSKLRLTSPPEPTVMTHVPYQVLA | 1024 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKGALDTNYCFRNLEENCCV | |
| | RPLYIDFRQDLGWKWVHEPKGYYANFCSGPCPYLRSADTTHSTVLGLY | |
| | NTLNPEASASPCCVPQDLEPLTILYYVGRTPKVEQLSNMVVKSCKCS | |

TABLE 10
Exemplary non-human amino acid sequences

| | | | SEQ ID |
|---|---|---|---|
| Protein | Species | Sequence | NO |
| proTGFβ1 | Mouse | LSTCKTIDMELVKRKRIEAIRGQILSKLRLASPPSQGEVPPGPLPEAVL | 1025 |
| | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRRALDTNYCFSSTEKNCCVRQLYIDFRKDLGWK | |
| | | WIHEPKGYHANFCLGPCPYIWSLDTQYSKVLALYNQHNPGASASPC | |
| | | CVPQALEPLPIVYYVGRKPKVEQLSNMIVRSCKCS | |
| proTGFβ1 | Cyno | LSTCKTIDMELVKRKRIEAIRGQILSKLRLASPPSQGEVPPGPLPEAVL | 1026 |
| | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRRALDTNYCFSSTEKNCCVRQLYIDFRKDLGWKWI | |
| | | HEPKGYHANFCLGPCPYIWSLDTQYSKVLALYNQHNPGASAAPCCV | |
| | | PQALEPLPIVYYVGRKPKVEQLSNMIVRSCKCS | |
| TGFβ1 LAP | Mouse | LSTSKTIDMELVKRKRIEAIRGQILSKLRLASPPSQGEVPPGPLPEAVL | 1027 |
| C4S | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRR | |
| TGFβ1 LAP | Cyno | LSTSKTIDMELVKRKRIEAIRGQILSKLRLASPPSQGEVPPGPLPEAVL | 1028 |
| C4S | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRR | |
| proTGFβ1 | Mouse | LSTSKTIDMELVKRKRIEAIRGQILSKLRLASPPSQGEVPPGPLPEAVL | 1029 |
| C4S D2G | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHGALDTNYCFSSTEKNCCVRQLYIDFRKDLGWKWI | |
| | | HEPKGYHANFCLGPCPYIWSLDTQYSKVLALYNQHNPGASASPCCV | |
| | | PQALEPLPIVYYVGRKPKVEQLSNMIVRSCKCS | |
| proTGFβ1 | Mouse | LSTSKTIDMELVKRKRIEAIRGQILSKLRLASPPSQGEVPPGPLPEAVL | 1030 |
| C4S | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRRALDTNYCFSSTEKNCCVRQLYIDFRKDLGWK | |
| | | WIHEPKGYHANFCLGPCPYIWSLDTQYSKVLALYNQHNPGASASPC | |
| | | CVPQALEPLPIVYYVGRKPKVEQLSNMIVRSCKCS | |
| proTGFβ1 | Cyno | LSTSKTIDMELVKRKRIEAIRGQILSKLRLASPPSQGEVPPGPLPEAVL | 1031 |
| C4S | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRRALDTNYCFSSTEKNCCVRQLYIDFRKDLGWKWI | |
| | | HEPKGYHANFCLGPCPYIWSLDTQYSKVLALYNQHNPGASAAPCCV | |
| | | PQALEPLPIVYYVGRKPKVEQLSNMIVRSCKCS | |
| proTGFβ1 | Cyno | LSTSKTIDMELVKRKRIEAIRGQILSKLRLASPPSQGEVPPGPLPEAVL | 1032 |
| C4S D2G | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHGALDTNYCFSSTEKNCCVRQLYIDFRKDLGWKWIH | |
| | | EPKGYHANFCLGPCPYIWSLDTQYSKVLALYNQHNPGASAAPCCVP | |
| | | QALEPLPIVYYVGRKPKVEQLSNMIVRSCKCS | |
| LTBP3 | CYNC | GPAGERGAGGGGALARERFKVVFAPVICKRTCLKGQCRDSCQQGS | 1033 |
| | | NMTLIGENGHSTDTLTGSGFRVVVCPLPCMNGGQCSSRNQCLCPP | |
| | | DFTGRFCQVPAGGAGGGTGGSGPGLSRAGALSTGALPPLAPEGDS | |
| | | VASKHAIYAVQVIADPPGPGEGPPAQHAAFLVPLGPGQISAEVQAPP | |
| | | PVVNVRVHHPPEASVQVHRIESSNAEGAAPSQHLLPHPKPSHPRPP | |
| | | TQKPLGRCFQDTLPKQPCGSNPLPGLTKQEDCCGSIGTAWGQSKC | |
| | | HKCPQLQYTGVQKPGPVRGEVGADCPQGYKRLNSTHCQDINECAM | |
| | | PGVCRHGDCLNNPGSYRCVCPPGHSLGPSRTQCIADKPEEKSLCF | |
| | | RLVSPEHQCQHPLTTRLTRQLCCCSVGKAWGARCQRCPADGTAAF | |
| | | KEICPAGKGYHILTSHQTLTIQGESDFSLFLHPDGPPKPQQLPESPS | |
| | | QAPPPEDTEEERGVTTDSPVSEERSVQQSHPTATTSPARPYPELIS | |
| | | RPSPPTMRWFLPDLPPSRSAVEIAPTQVTETDECRLNQNICGHGEC | |
| | | VPGPPDYSCHCNPGYRSHPQHRYCVDVNECEAEPCGPGRGICMN | |
| | | TGGSYNCHCNRGYRLHVGAGGRSCVDLNECAKPHLCGDGGFCINF | |
| | | PGHYKCNCYPGYRLKASRPPVCEDIDECRDPSSCPDGKCENKPGS | |
| | | FKCIACQPGYRSQGGGACRDVNECAEGSPCSPGWCENLPGSFRC | |
| | | TCAQGYAPAPDGRSCVDVDECEAGDVCDNGICTNTPGSFQCQCLS | |
| | | GYHLSRDRSHCEDIDECDFPAACIGGDCINTNGSYRCLCPQGHRLV | |
| | | GGRKCQDIDECTQDPGLCLPHGACKNLQGSYVCVCDEGFTPTQDQ | |
| | | HGCEEVEQPHHKKECYLNFDDTVFCDSVLATNVTQQECCCSLGAG | |
| | | WGDHCEIYPCPVYSSAEFHSLCPDGKGYTQDNNIVNYGIPAHRDIDE | |
| | | CMLFGAEICKEGKCVNTQPGYECYCKQGFYYDGNLLECVDVDECL | |
| | | DESNCRNGVCENTRGGYRCACTPPAEYSPAQRQCLSPEEMDVDE | |
| | | CQDPAACRPGRCVNLPGSYRCECRPPWVPGPSGRDCQLPESPAE | |
| | | RAPERRDVCWSQRGEDGMCAGPQAGPALTFDDCCCRQGRGWGA | |
| | | QCRPCPPRGAGSQCPTSQSESNSFWDTSPLLLGKPRRDEDSSEED | |
| | | SDECRCVSGRCVPRPGGAVCECPGGFQLDASRARCVDIDECRELN | |
| | | QRGLLCKSERCVNTSGSFRCVCKAGFARSRPHGACVPQRRR | |
| LTBP3 | Mouse | GPAGERGTGGGGALARERFKVVFAPVICKRTCLKGQCRDSCQQGS | 1034 |
| | | NMTLIGENGHSTDTLTGSAFRVVVCPLPCMNGGQCSSRNQCLCPP | |
| | | DFTGRFCQVPAAGTGAGTGSSGPGLARTGAMSTGPLPPLAPEGES | |
| | | VASKHAIYAVQVIADPPGPGEGPPAQHAAFLVPLGPGQISAEVQAPP | |
| | | PVVNVRVHHPPEASVQVHRIEGPNAEGPASSQHLLPHPKPPHPRPP | |
| | | TQKPLGRCFQDTLPKQPCGSNPLPGLTKQEDCCGSIGTAWGQSKC | |
| | | HKCPQLQYTGVQKPVPVRGEVGADCPQGYKRLNSTHCQDINECAM | |
| | | PGNVCHGDCLNNPGSYRCVCPPGHSLGPLAAQCIADKPEEKSLCFR | |
| | | LVSTEHQCQHPLTTRLTRQLCCCSVGKAWGARCQRCPADGTAAFK | |
| | | EICPGKGYHILTSHQTLTIQGESDFSLFLHPDGPPKPQQLPESPSRAP | |
| LTBP1S | Cyno | PLEDTEEERGVTMDPPVSEERSVQQSHPTTTTSPPRPYPELISRPSP | 1035 |
| | | PTFHRFLPDLPPSRSAVEIAPTQVTETDECRLNQNICGHGQCVPGPS | |
| | | DYSCHCNAGYRSHPQHRYCVDVNECEAEPCGPGKGICMNTGGSY | |
| | | NCHCNRGYRLHVGAGGRSCVDLNECAKPHLCGDGGFCINFPGHYK | |
| | | CNCYPGYRLKASRPPICEDIDECRDPSTCPDGKCENKPGSFKCIAC | |
| | | QPGYRSQGGGACRDVNECSEGTPCSPGWCENLPGSYRCTCAQYE | |
| | | PAQDGLSCIDVDECEAGKVCQDGICTNTPGSFQCQCLSGYHLSRDR | |
| | | SRCEDIDECDFPAACIGGDCINTNGSYRCLCPLGHRLVGGRKCKKDI | |
| | | DECSQDPGLCLPHACENLQGSYVCVCDEGFTLTQDQHGCEEVEQP | |
| | | HHKKECYLNFDDTVFCDSVLATNVTQQECCCSLGAGWGDHCEIYP | |
| | | CPVYSSAEFHSLVPDGKRLHSGQQHCELCIPAHRDIDECILFGAEICK | |
| | | EGKCVNTQPGYECYCKQGFYYDGNLLECVDVDECLDESNCRNGVC | |
| | | ENTRGGYRCACTPPAEYSPAQAQCLIPERWSTPQRDVKCAGASEE | |
| | | RTACVWGPWAGPALTFDDCCCRQPRLGTQCRPCPPRGTGSQCPT | |
| | | SQSESNSFWDTSPLLLGKSPRDEDSSEEDSDECRCVSGRCVPRPG | |
| | | GAVCECPGGFQLDASRARCVDIDECRELNQRGLLCKSERCVNTSG | |
| | | SFRCVCKAGFTRSRPHGPACLSAAADDAAIAHTSVIDHRGYFH | |
| | | NHTGRIKVVFTPSICKVTCTKGSCQNSCEKGNTTTLISENGHAADTLT | |
| | | ATNFRVVLCHLPCMNGGQCSSRDKCQCPPNFTGKLCQIPVHGASV | |
| | | PKLYQHSQQPGKALGTHVIHSTHTLPLTVTSQQGVKVKFPPNIVNIH | |
| | | VKHPPEASVQIHQVSRIDGPTGQKTKEAQPGQSQVSYQGLPVQKTQ | |
| | | TIHSTYSHQQVIPHVYPVAAKTQLGRCFQETIGSQCGKALPGLSKQE | |
| | | DCCGTVGTSWGFNKCQKCPKKPSYHGYNQMMECLPGYKRVNNTF | |
| | | CQDINECQLQGVCPNGECLNTMGSYRCTCKIGFGPDPTFSSCVPDP | |
| | | PVISEEKGPCYRLVSSGRQCMHPLSVHLTKQLCCCSVGKAWGPHC | |
| | | EKCPLPGTAAFKEICPGGMGYTVSGVHRRRPIHHHVGKGPVFVKPK | |
| | | NTQPVAKSTHPPPLPAKEEPVEALTFSREHGPGVAEPEVATAPPEK | |
| | | EIPSLDQEKTKLEPGQPQLSPGISTIHLHPQFPVVIEKTSPPVPVEVAP | |
| | | EASTSSASQVIAPTQVTEINECTVNPDICGAGHCINLPVRYTCICYEG | |
| | | YKFSEQQRKCVDIDECTQVQHLCSQGRCENTEGSFLCICPAGFMAS | |
| | | EEGTNCIDVDECLRPDVCGEGHCVNTVGAFRCEYCDSGYRMTQRG | |
| | | RCEDIDECLNPSTCPDEQCVNSPGSYQCVPCTEGFRGWNGQCLDV | |
| | | DECLEPNVCTNGDCSNLEGSYMCSCHKGYTRTPDHKHCKDIDECQ | |
| | | QGNLCVNGQCKNTEGSFRCTCGQGYQLSAAKDQCEDIDECQHHHL | |
| | | CAHGQCRNTEGSFQCVCDQGYRASGLGDHCEDINECLEDKSVCQR | |
| | | GDCINTAGSYDCTCPDGFQLDDNKTCQDINECEHPGLCGPQGECL | |
| | | NTEGSFHCVCQQGFSISADGRTCEDIDECVNNTVCDSHGFCDNTAG | |
| | | SFRCLCYQGFQAPQDGQGCVDVNECELLSGVCGEAFCENVEGSFL | |
| | | CVCADENQEYSPMTGQCRSRTSTDLDVEQPKEEKKECYYNLNDAS | |
| | | LCDNVLAPNVTKQECCCTSGAGWGDNCEIFPCPVLGTAEFTEMCPK | |
| | | GKGFVPAGESSSEAGGENYKDADECLLFGQEICKNGFCLNTRPGYE | |
| | | CYCKQGTYYDPVKLQCFDMDECQDPSSCIDGQCVNTEGSYNCFCT | |
| | | HPMVLDASEKRCIRPAESNEQIEETDVYQDLCWEHLSDEYVCSRPL | |
| | | VGKQTTYTECCCLYGEAWGMQCALCPMKDSDDYAQLCNIPVTGRR | |
| | | QPYGRDALVDFSEQYAPEADPYFIQDRFLNSFEELQAEECGILNGCE | |
| | | NGRCVRVQEGYTCDCFDGYHLDTAKMTCVDVNECDELNNRMSLCK | |
| | | NAKCINTEGSYKCLCLPGYVPSDKPNYCTPLNTALNLEKDSDLE | |
| LTBP1S | mouse | NHTGRIKVVFTPSICKVTCTKGNCQNSCQKGNTTTLISENGHAADTL | 1036 |
| | | TATNFRVVICHLPCMNGGQCSSRDKCQCPPNFTGKLCQIPVLGASM | |
| | | PKLYQHAQQQGKALGSHVIHSTHTLPLTMTSQQGVKVKFPPNIVNIH | |
| | | VKHPPEASVQIHQVSRIDSPGGQKVKEAQPGQSQVSYQGLPVQKT | |
| | | QTVHSTYSHQQLIPHVYPVAAKTQLGRCFQETIGSQCGKALPGLSK | |
| | | QEDCCGTVGTSWGFNKCQKCPKKQSYHGYTQMMECLQGYKRVN | |
| | | NTFCQDINECQLQGVCPNGECLNTMGSYRCSCKMGFGPDPTFSSC | |
| | | VPDPPVISEEKGPCYRLVSPGRHCMHPLSVHLTKQICCCSVGKAWG | |
| | | PHCEKCPLPGTAAFKEICPGGMGYTVSGVHRRRPIHQHIGKEAVYV | |
| | | KPKNTQPVAKSTHPPPLPAKEEPVEALTSSWEHGPRGAEPEVVTAP | |
| | | PEKEIPSLDQEKTRLEPGQPQLSPGVSTIHLHPQFPVVVEKTSPPVP | |
| | | VEVAPEASTSSASQVIAPTQVTEINECTVNPDICGAGHCINLPVRYTC | |
| | | ICYEGYKFSEQLRKCVDIDECAQVRHLCSQGRCENTEGSFLCVCPA | |
| | | GFMASEEGTNCIDVDECLRPDMCRDGRCINTAGAFRCEYCDSGYR | |
| | | MSRRGYCEDIDECLKPSTCPEEQCVNTPGSYQCVPCTEGFRGWNG | |
| | | QCLDVDECLQPKVCTNGSCTNLEGSYMCSCHRGYSPTPDHRHCQ | |
| | | DIDECQQGNLCMNGQCRNTDGSFRCTCGQGYQLSAAKDQCEDIDE | |
| | | CEHHHLCSHGQCRNTEGSFQCVCNQGYRASVLGDHCEDINECLED | |
| | | SSVCQGGDCINTAGSYDCTCPDGFQLNDNKGCQDINECAQPGLCG | |
| | | SHGECLNTQGSFHCVCEQGFSISADGRTCEDIDECVNNTVCDSHGF | |
| | | CDNTAGSFRCLCYQGFQAPQDGQGCVDVNECELLSGVCGEAFCE | |
| | | NVEGSFLCVCADENQEYSPMTGQCRSRVTEDSGVDRQPREEKKEC | |
| | | YYNLNDASLCDNVLAPNVTKQECCCTSGAGWGDNCEIFPCPVQGT | |
| | | AEFTEMCPRGKGLVPAGESSYDTGGENYKDADECLLFGEEICKNGY | |
| | | CLNTQPGYECYCKQGTYYDPVKLQCFDMDECQDPNSCIDGQCVNT | |
| | | EGSYNCFCTHPMVLDASEKRCVQPTESNEQIEETDVYQDLCWEHLS | |
| | | EEYVCSRPLVGKQTTYTECCCLYGEAWGMQCALCPMKDSDDYAQL | |
| | | CNIPVTGRRRPYGRDALVDFSEQYGPETDPYFIQDRFLNSFEELQAE | |
| | | ECGILNGCENGRCVRVQEGYTCDCFDGYHLDMAKMTCVDVNECSE | |
| | | LNNRMSLCKNAKCINTEGSYKCLCLPGYIPSDKPNYCTPLNSALNLD | |
| | | KESDLE | |
| GARP | mouse | ISQRREQVPCRTVNKEALCHGLGLLQVPSVLSLDIQALYLSGNQLQSI | 1037 |
| | | LVSPLGFYTALRHLDLSDNQISFLQAGVFQALPYLEHLNLAHNRLAT | |
| | | GMALNSGGLGRLPLLVSLDLSGNSLHGNLVERLLGETPRLRTLSLAE | |
| | | NSLTRLARHTFWGMPAVEQLDLHSNVLMDIEDGAFEALPHLTHLNLS | |
| | | RNSLTCISDFSLQQLQVLDLSCNSIEAFQTAPEPQAQFQLAWLDLRE | |
| | | NKLLHFPDLAVFPRLIYLNVSNNLIQLPAGLPRGSEDLHAPSEGWSA | |
| | | SPLSNPSRNASTHPLSQLLNLDLSYNEIELVPASFLEHLTSLRFLNLS | |
| | | RNCLRSFEARQVDSLPCLVLLDLSHNVLEALELGTKVLGSLQTLLLQ | |
| | | DNALQELPPYTFASLASLQRLNLQGNQVSPCGGPAEPGPPGCVDFS | |
| | | GIPTLHVLNMAGNSMGMLRAGSFLHTPLTELDLSTNPGLDVATGALV | |
| | | GLEASLEVLELQGNGLTVLRVDLPCFLRLKRLNLAENQLSHLPAWTR | |
| | | AVSLEVLDLRNNSFSLLPGNAMGGLETSLRRLYLQGNPLSCCGNGW | |
| | | LAAQLHQGRVDVDATQDLICRFGSQEELSLSLVRPEDCEKGGLKNV | |
| | | NLILLLSFTLVSAIVLTTLATICFLRRQKLSQQYKA | |
| sGARP | mouse | ISQRREQVPCRTVNKEALCHGLGLLQVPSVLSLDIQALYLSGNQLQSI | 1038 |
| | | LVSPLGFYTALRHLDLSDNQISFLQAGVFQALPYLEHLNLAHNRLAT | |
| | | GMALNSGGLGRLPLLVSLDLSGNSLHGNLVERLLGETPRLRTLSLAE | |
| | | NSLTRLARHTFWGMPAVEQLDLHSNVLMDIEDGAFEALPHLTHLNLS | |
| | | RNSLTCISDFSLQQLQVLDLSCNSIEAFQTAPEPQAQFQLAWLDLRE | |
| | | NKLLHFPDLAVFPRLIYLNVSNNLIQLPAGLPRGSEDLHAPSEGWSA | |
| | | SPLSNPSRNASTHPLSQLLNLDLSYNEIELVPASFLEHLTSLRFLNLS | |
| | | RNCLRSFEARQVDSLPCLVLLDLSHNVLEALELGTKVLGSLQTLLLQ | |
| | | DNALQELPPYTFASLASLQRLNLQGNQVSPCGGPAEPGPPGCVDFS | |
| | | GIPTLHVLNMAGNSMGMLRAGSFLHTPLTELDLSTNPGLDVATGALV | |
| | | GLEASLEVLELQGNGLTVLRVDLPCFLRLKRLNLAENQLSHLPAWTR | |
| | | AVSLEVLDLRNNSFSLLPGNAMGGLETSLRRLYLQGNPLSCCGNGW | |
| | | LAAQLHQGRVDVDATQDLICRFGSQEELSLSLVRPEDCEKGGLKNV | |
| | | N | |

In some embodiments, antigenic protein complexes (e.g., a LTBP-TGFβ1 complex) may comprise one or more presenting molecules, such as LTBP proteins (e.g., LTBP1, LTBP2, LTBP3, and LTBP4), GARP proteins, LRRC33 proteins, or fragment(s) thereof. Typically, a minimum required fragment suitable for carrying out the embodiments disclosed herein includes at least 50 amino acids, preferably at least 100 amino acids, of a presenting molecule protein, comprising at least two cysteine residues capable of forming disulfide bonds with a proTGFβ1l complex. Specifically, these Cys residues form covalent bonds with Cysteine resides present near the N-terminus of each monomer of the proTGFβ1l complex. In the three-dimensional structure of a proTGFβ1l dimer complex, the N-terminal so-called “Alpha-1 Helix” of each monomer comes in close proximity to each other, setting the distance between the two cysteine residues (one from each helix) required to form productive covalent bonds with a corresponding pair of cysteines present in a presenting molecule (see, for example, Cuende et al., (2015) Sci. Trans. Med. 7: 284ra56). Therefore, when a fragment of a presenting molecule is used to form an LLC in the screening process (e.g., immunization, library screening, identification, and selection), such fragment should include the cysteine residues separated by the right distance, which will allow proper disulfide bond formation with a proTGFβ1 complex in order to preserve correct conformation of the resulting LLC. LTBPs (e.g., LTBP1, LTBP3 and LTBP4), for example, may contain “cysteine-rich domains” to mediate covalent interactions with proTGFβ1.

In some embodiments, antigenic protein complexes may be so-called a small latent complex (or SLC), comprised of a dimeric complex of the LAP (or prodomain) and a growth factor domain. In other embodiments, antigenic protein complexes may be so-called a large latent complex (or LLC) which further comprises a presenting molecule bound to a SLC. Presenting molecules include LTBP proteins (e.g., LTBP1, LTBP2, LTBP3, and LTBP4), GARP proteins, LRRC33 proteins, or fragment(s) thereof. When LLCs are used as antigenic protein complexes, typically, a minimum required fragment suitable for carrying out the embodiments disclosed herein includes at least 50 amino acids, preferably at least 100 amino acids, of a presenting molecule protein, comprising at least two cysteine residues capable of forming disulfide bonds with a proTGFβ1 complex.

An antibody, or antigen binding portion thereof, as described herein, is capable of binding to a LTBP1-TGFβ1 complex. In some embodiments, the LTBP1 protein is a naturally-occurring protein or fragment thereof. In some embodiments, the LTBP1 protein is a non-naturally occurring protein or fragment thereof. In some embodiments, the LTBP1 protein is a recombinant protein. Such recombinant LTBP1 protein may comprise LTBP1, alternatively spliced variants thereof and/or fragments thereof. Recombinant LTBP1 proteins may also be modified to comprise one or more detectable labels. In some embodiments, the LTBP1 protein comprises a leader sequence (e.g., a native or non-native leader sequence). In some embodiments, the LTBP1 protein does not comprise a leader sequence (i.e., the leader sequence has been processed or cleaved). Such detectable labels may include, but are not limited to biotin labels, polyhistidine tags, myc tags, HA tags and/or fluorescent tags. In some embodiments, the LTBP1 protein is a mammalian LTBP1 protein. In some embodiments, the LTBP1 protein is a human, a monkey, a mouse, or a rat LTBP1 protein. In some embodiments, the LTBP1 protein comprises an amino acid sequence as set forth in SEQ ID NOs: 1035 and 1036 in Table 10. In some embodiments, the LTBP1 protein comprises an amino acid sequence as set forth in SEQ ID NO: 1039 in Table 12. In some embodiments, the LTBP1 protein comprises a leader sequence (e.g., a native or non-native leader sequence). In some embodiments, the LTBP1 protein does not comprise a leader sequence (i.e., the leader sequence has been processed or cleaved). In some embodiments, the LTBP1 protein comprises an amino acid sequence as set forth in SEQ ID NOs: 46 and 47. In some embodiments, the LTBP1 protein comprises an amino acid sequence as set forth in SEQ ID NO: 50.

An antibody, or antigen binding portion thereof, as described herein, is capable of binding to a LTBP3-TGFβ1 complex. In some embodiments, the LTBP3 protein is a naturally-occurring protein or fragment thereof. In some embodiments, the LTBP3 protein is a non-naturally occurring protein or fragment thereof. In some embodiments, the LTBP3 protein is a recombinant protein. Such recombinant LTBP3 protein may comprise LTBP3, alternatively spliced variants thereof and/or fragments thereof. In some embodiments, the LTBP3 protein comprises a leader sequence (e.g., a native or non-native leader sequence). In some embodiments, the LTBP3 protein does not comprise a leader sequence (i.e., the leader sequence has been processed or cleaved). Recombinant LTBP3 proteins may also be modified to comprise one or more detectable labels. Such detectable labels may include, but are not limited to biotin labels, polyhistidine tags, myc tags, HA tags and/or fluorescent tags. In some embodiments, the LTBP3 protein is a mammalian LTBP3 protein. In some embodiments, the LTBP3 protein is a human, a monkey, a mouse, or a rat LTBP3 protein. In some embodiments, the LTBP3 protein comprises an amino acid sequence as set forth in SEQ ID NOs: 1033 and 1034 in Table 10. In some embodiments, the LTBP1 protein comprises an amino acid sequence as set forth in SEQ ID NO: 1040 in Table 12.

An antibody, or antigen binding portion thereof, as described herein, is capable of binding to a GARP-TGFβ1 complex. In some embodiments, the GARP protein is a naturally-occurring protein or fragment thereof. In some embodiments, the GARP protein is a non-naturally occurring protein or fragment thereof. In some embodiments, the GARP protein is a recombinant protein. Such a GARP may be recombinant, referred to herein as recombinant GARP. Some recombinant GARPs may comprise one or more modifications, truncations and/or mutations as compared to wild type GARP. Recombinant GARPs may be modified to be soluble. In some embodiments, the GARP protein comprises a leader sequence (e.g., a native or non-native leader sequence). In some embodiments, the GARP protein does not comprise a leader sequence (i.e., the leader sequence has been processed or cleaved). In other embodiments, recombinant GARPs are modified to comprise one or more detectable labels. In further embodiments, such detectable labels may include, but are not limited to biotin labels, polyhistidine tags, flag tags, myc tags, HA tags and/or fluorescent tags. In some embodiments, the GARP protein is a mammalian GARP protein. In some embodiments, the GARP protein is a human, a monkey, a mouse, or a rat GARP protein. In some embodiments, the GARP protein comprises an amino acid sequence as set forth in SEQ ID NOs: 1037-1038 in Table 10. In some embodiments, the GARP protein comprises an amino acid sequence as set forth in SEQ ID NOs: 1041 and 1042 in Table 12. In some embodiments, the antibodies, or antigen binding portions thereof, described herein do not bind to TGFβ1 in a context-dependent manner, for example binding to TGFβ1 would only occur when the TGFβ1 molecule was complexed with a specific presenting molecule, such as GARP. Instead, the antibodies, and antigen-binding portions thereof, bind to TGFβ1 in a context-independent manner. In other words, the antibodies, or antigen-binding portions thereof, bind to TGFβ1 when bound to any presenting molecule: GARP, LTBP1, LTBP3, and/or LRRC33.

An antibody, or antigen binding portion thereof, as described herein, is capable of binding to a LRRC33-TGFβ1 complex. In some embodiments, the LRRC33 protein is a naturally-occurring protein or fragment thereof. In some embodiments, the LRRC33 protein is a non-naturally occurring protein or fragment thereof. In some embodiments, the LRRC33 protein is a recombinant protein. Such a LRRC33 may be recombinant, referred to herein as recombinant LRRC33. Some recombinant LRRC33 proteins may comprise one or more modifications, truncations and/or mutations as compared to wild type LRRC33. Recombinant LRRC33 proteins may be modified to be soluble. For example, in some embodiments, the ectodomain of LRRC33 may be expressed with a C-terminal His-tag in order to express soluble LRRC33 protein (sLRRC33; see, e.g., SEQ ID NO: 1073). In some embodiments, the LRRC33 protein comprises a leader sequence (e.g., a native or non-native leader sequence). In some embodiments, the LRRC33 protein does not comprise a leader sequence (i.e., the leader sequence has been processed or cleaved). In other embodiments, recombinant LRRC33 proteins are modified to comprise one or more detectable labels. In further embodiments, such detectable labels may include, but are not limited to biotin labels, polyhistidine tags, flag tags, myc tags, HA tags and/or fluorescent tags. In some embodiments, the LRRC33 protein is a mammalian LRRC33 protein. In some embodiments, the LRRC33 protein is a human, a monkey, a mouse, or a rat LRRC33 protein. In some embodiments, the LRRC33 protein comprises an amino acid sequence as set forth in SEQ ID NOs: 1072, 1073, and 1074 in Table 12.

TABLE 11
Exemplary LTBP amino acid sequences

| | | SEQ |
|---|---|---|
| Protein | Sequence | ID NO |
| LTBP1S | NHTGRIKVVFTPSICKVTCTKGSCQNSCEKGNTTTLISENGHAADTLT | 1039 |
| | ATNFRVVICHLPCMNGGQCSSRDKCQCPPNFTGKLCQIPVHGASVP | |
| | KLYQHSQQPGKALGTHVIHSTHTLPLTVTSQQGVKVKFPPNIVNIHVK | |
| | HPPEASVQIHQVSRIDGPTGQKTKEAQPGQSQVSYQGLPVQKTQTIH | |
| | STYSHQQVIPHVYPVAAKTQLGRCFQETIGSQCGKALPGLSKQEDCC | |
| | GTVGTSWGFNKCQKCPKKPSYHGYNQMMECLPGYKRVNNTFCQDI | |
| | NECQLQGVCPNGECLNTMGSYRCTCKIGFGPDPTFSSCVPDPPVISE | |
| | EKGPCYRLVSSGRQCMHPLSVHLTKQLCCCSVGKAWGPHCEKCPL | |
| | PGTAAFKEICPGGMGYTVSGVHRRRPIHHHVGKGPVFVKPKNTQPV | |
| | AKSTHPPPLPAKEEPVEALTFSREHGPGVAEPEVATAPPEKEIPSLDQ | |
| | EKTKLEPGQPQLSPGISTIHLHPQFPVVIEKTSPPVPVEVAPEASTSSA | |
| | SQVIAPTQVTEINECTVNPDICGAGHCINLPVRYTCICYEGYRFSEQQ | |
| | RKCVDIDECTQVQHLCSQGRCENTEGSFLCICPAGFMASEEGTNCID | |
| | VDECLRPDVCGEGHCVNTVGAFRCEYCDSGYRMTQRGRCEDIDECL | |
| | NPSTCPDEQCVNSPGSYQCVPCTEGFRGWNGQCLDVDECLEPNVC | |
| | ANGDCSNLEGSYMCSCHKGYTRTPDHKHCRDIDECQQGNLCVNGQ | |
| | CKNTEGSFRCTCGQGYQLSAAKDQCEDIDECQHRHLCAHGQCRNT | |
| | EGSFQCVCDQGYRASGLGDHCEDINECLEDKSVCQRGDCINTAGSY | |
| | DCTCPDGFQLDDNKTCQDINECEHPGLCGPQGECLNTEGSFHCVCQ | |
| | QGFSISADGRTCEDIDECVNNTVCDSHGFCDNTAGSFRCLCYQGFQ | |
| | APQDGQGCVDVNECELLSGVCGEAFCENVEGSFLCVCADENQEYSP | |
| | MTGQCRSRTSTDLDVDVDQPKEEKKECYYNLNDASLCDNVLAPNVT | |
| | KQECCCTSGVGWGDNCEIFPCPVLGTAEFTEMCPKGKGFVPAGESS | |
| | SEAGGENYKDADECLLFGQEICKNGFCLNTRPGYECYCKQGTYYDP | |
| | VKLQCFDMDECQDPSSCIDGQCVNTEGSYNCFCTHPMVLDASEKRC | |
| | IRPAESNEQIEETDVYQDLCWEHLSDEYVCSRPLVGKQTTYTECCCL | |
| | YGEAWGMQCALCPLKDSDDYAQLCNIPVTGRRQPYGRDALVDFSEQ | |
| | YTPEADPYFIQDRFLNSFEELQAEECGILNGCENGRCVRVQEGYTCD | |
| | CFDGYHLDTAKMTCVDVNECDELNNRMSLCKNAKCINTDGSYKCLCL | |
| | PGYVPSDKPNYCTPLNTALNLEKDSDLE | |
| LTBP3 | GPAGERGAGGGGALARERFKVVFAPVICKRTCLKGQCRDSCQQGS | 1040 |
| | NMTLIGENGHSTDTLTGSGFRVVVCPLPCMNGGQCSSRNQCLCPPD | |
| | FTGRFCQVPAGGAGGGTGGSGPGLSRTGALSTGALPPLAPEGDSVA | |
| | SKHAIYAVQVIADPPGPGEGPPAQHAAFLVPLGPGQISAEVQAPPPVV | |
| | NVRVHHPPEASVQVHRIESSNAESAAPSQHLLPHPKPSHPRPPTQKP | |
| | LGRCFQDTLPKQPCGSNPLPGLTKQEDCCGSIGTAWGQSKCHKCPQ | |
| | LQYTGVQKPGPVRGEVGADCPQGYKRLNSTHCQDINECAMPGVCR | |
| | HGDCLNNPGSYRCVCPPGHSLGPSRTQCIADKPEEKSLCFRLVSPEH | |
| | QCQHPLTTRLTRQLCCCSVGKAWGARCQRCPTDGTAAFKEICPAGK | |
| | GYHILTSHQTLTIQGESDFSLFLHPDGPPKPQQLPESPSQAPPPEDTE | |
| | EERGVTTDSPVSEERSVQQSHPTATTTPARPYPELISRPSPPTMRWF | |
| | LPDLPPSRSAVEIAPTQVTETDECRLNQNICGHGECVPGPPDYSCHC | |
| | NPGYRSHPQHRYCVDVNECEAEPCGPGRGICMNTGGSYNCHCNRG | |
| | YRLHVGAGGRSCVDLNECAKPHLCGDGGFCINFPGHYKCNCYPGYR | |
| | LKASRPPVCEDIDECRDPSSCPDGKCENKPGSFKCIACQPGYRSQG | |
| | GGACRDVNECAEGSPCSPGWCENLPGSFRCTCAQGYAPAPDGRSC | |
| | LDVDECEAGDVCDNGICSNTPGSFQCQCLSGYHLSRDRSHCEDIDE | |
| | CDFPAACIGGDCINTNGSYRCLCPQGHRLVGGRKCQDIDECSQDPSL | |
| | CLPHGACKNLQGSYVCVCDEGFTPTQDQHGCEEVEQPHHKKECYL | |
| | NFDDTVFCDSVLATNVTQQECCCSLGAGWGDHCEIYPCPVYSSAEF | |
| | HSLCPDGKGYTQDNNIVNYGIPAHRDIDECMLFGSEICKEGKCVNTQ | |
| | PGYECYCKQGFYYDGNLLECVDVDECLDESNCRNGVCENTRGGYR | |
| | CACTPPAEYSPAQRQCLSPEEMDVDECQDPAACRPGRCVNLPGSY | |
| | RCECRPPWVPGPSGRDCQLPESPAERAPERRDVCWSQRGEDGMC | |
| | AGPLAGPALTFDDCCCRQGRGWGAQCRPCPPRGAGSHCPTSQSES | |
| | NSFWDTSPLLLGKPPRDEDSSEEDSDECRCVSGRCVPRPGGAVCEC | |
| | PGGFQLDASRARCVDIDECRELNQRGLLCKSERCVNTSGSFRCVCK | |
| | AGFARSRPHGACVPQRRR | |

TABLE 12
Exemplary GARP and LRRC33 amino acid sequences

| | | SEQ |
|---|---|---|
| Protein | Sequence | ID NO |
| GARP | AQHQDKVPCKMVDKKVSCQVLGLLQVPSVLPPDTETLDLSGNQLRSILA | 1041 |
| | SPLGFYTALRHLDLSTNEISFLQPGAFQALTHLEHLSLAHNRLAMATALS | |
| | AGGLGPLPRVTSLDLSGNSLYSGLLERLLGEAPSLHTLSLAENSLTRLTR | |
| | HTFRDMPALEQLDLHSNVLMDIEDGAFEGLPRLTHLNLSRNSLTCISDFS | |
| | LQQLRVLDLSCNSIEAFQTASQPQAEFQLTWLDLRENKLLHFPDLAALP | |
| | RLIYLNLSNNLIRLPTGPPQDSKGIHAPSEGWSALPLSAPSGNASGRPLS | |
| | QLLNLDLSYNEIELIPDSFLEHLTSLCFLNLSRNCLRTFEARRLGSLPCLM | |
| | LLDLSHNALETLELGARALGSLRTLLLQGNALRDLPPYTFANLASLQRLN | |
| | LQGNRVSPCGGPDEPGPSGCVAFSGITSLRSLSLVDNEIELLRAGAFLH | |
| | TPLTELDLSSNPGLEVATGALGGLEASLEVLALQGNGLMVLQVDLPCFIC | |
| | LKRLNLAENRLSHLPAWTQAVSLEVLDLRNNSFSLLPGSAMGGLETSLR | |
| | RLYLQGNPLSCCGNGWLAAQLHQGRVDVDATQDLICRFSSQEEVSLSH | |
| | VRPEDCEKGGLKNINLIIILTFILVSAILLTTLAACCCVRRQKFNQQYKA | |
| sGARP | AQHQDKVPCKMVDKKVSCQVLGLLQVPSVLPPDTETLDLSGNQLRSILA | 1042 |
| | SPLGFYTALRHLDLSTNEISFLQPGAFQALTHLEHLSLAHNRLAMATALS | |
| | AGGLGPLPRVTSLDLSGNSLYSGLLERLLGEAPSLHTLSLAENSLTRLTR | |
| | HTFRDMPALEQLDLHSNVLMDIEDGAFEGLPRLTHLNLSRNSLTCISDFS | |
| | LQQLRVLDLSCNSIEAFQTASQPQAEFQLTWLDLRENKLLHFPDLAALP | |
| | RLIYLNLSNNLIRLPTGPPQDSKGIHAPSEGWSALPLSAPSGNASGRPLS | |
| | QLLNLDLSYNEIELIPDSFLEHLTSLCFLNLSRNCLRTFEARRLGSLPCLM | |
| | LLDLSHNALETLELGARALGSLRTLLLQGNALRDLPPYTFANLASLQRLN | |
| | LQGNRVSPCGGPDEPGPSGCVAFSGITSLRSLSLVDNEIELLRAGAFLH | |
| | TPLTELDLSSNPGLEVATGALGGLEASLEVLALQGNGLMVLQVDLPCFIC | |
| | LKRLNLAENRLSHLPAWTQAVSLEVLDLRNNSFSLLPGSAMGGLETSLR | |
| | RLYLQGNPLSCCGNGWLAAQLHQGRVDVDATQDLICRFSSQEEVSLSH | |
| | VRPEDCEKGGLKNIN | |
| LRRC33 (also known as | MELLPLWLCLGFHFLTVGWRNRSGTATAASQGVCKLVGGAADCRGQ | 1072 |
| NRROS; Uniprot | SLASVPSSLPPHARMLTLDANPLKTLWNHSLQPYPLLESLSLHSCHLERI | |
| | SRGAFQEQGHLRSLVLGDNCLSENYEETAAALHALPGLRRLDLSGNAL | |
| | TEDMAALMLQNLSSLRSVSLAGNTIMRLDDSVFEGLERLRELDLQRNYI | |
| | FEIEGGAFDGLAELRHLNLAFNNLPCIVDFGLTRLRVLNVSYNVLEWFLA | |
| | TGGEAAFELETLDLSHNQLLFFPLLPQYSKLRTLLLRDNNMGFYRDLYN | |
| | TSSPREMVAQFLLVDGNVTNITTVSLWEEFSSSDLADLRFLDMSQNQF | |
| | QYLPDGFLRKMPSLSHLNLHQNCLMTLHIREHEPPGALTELDLSHNQLS | |
| | ELHLAPGLASCLGSLRLFNLSSNQLLGVPPGLFANARNITTLDMSHNQIS | |
| | LCPLPAASDRVGPPSCVDFRNMASLRSLSLEGCGLGALPDCPFQGTSL | |
| | TYLDLSSNWGVLNGSLAPLQDVAPMLQVLSLRNMGLHSSFMALDFSGF | |
| | GNLRDLDLSGNCLTTFPRFGGSLALETLDLRRNSLTALPQKAVSEQLSR | |
| | GLRTIYLSQNPYDCCGVDGWGALQHGQTVADWAMVTCNLSSKIIRVTE | |
| | LPGGVPRDCKWERLDLGLLYLVLILPSCLTLLVACTVIVLTFKKPLLQVIK | |
| | SRCHWSSVY | |
| Accession No. Q86YC3) | * Native signal peptide is depicted in bold font. | |
| soluble LRRC33 | MDMRVPAQLLGLLLLWFSGVLGWRNRSGTATAASQGVCKLVGGAAD | 1073 |
| | CRGQSLASVPSSLPPHARMLTLDANPLKTLWNHSLQPYPLLESLSLHSC | |
| | HLERISRGAFQEQGHLRSLVLGDNCLSENYEETAAALHALPGLRRLDLS | |
| | GNALTEDMAALMLQNLSSLRSVSLAGNTIMRLDDSVFEGLERLRELDLQ | |
| | RNYIFEIEGGAFDGLAELRHLNLAFNNLPCIVDFGLTRLRVLNVSYNVLE | |
| | WFLATGGEAAFELETLDLSHNQLLFFPLLPQYSKLRTLLLRDNNMGFYR | |
| | DLYNTSSPREMVAQFLLVDGNVTNITTVSLWEEFSSSDLADLRFLDMSQ | |
| | NQFQYLPDGFLRKMPSLSHLNLHQNCLMTLHIREHEPPGALTELDLSHN | |
| | QLSELHLAPGLASCLGSLRLFNLSSNQLLGVPPGLFANARNITTLDMSH | |
| | NQISLCPLPAASDRVGPPSCVDFRNMASLRSLSLEGCGLGALPDCPFQ | |
| | GTSLTYLDLSSNWGVLNGSLAPLQDVAPMLQVLSLRNMGLHSSFMALD | |
| | FSGFGNLRDLDLSGNCLTTFPRFGGSLALETLDLRRNSLTALPQKAVSE | |
| | QLSRGLRTIYLSQNPYDCCGVDGWGALQHGQTVADWAMVTCNLSSKII | |
| | RVTELPGGVPRDCKWERLDLGLHHHHHH | |
| (sLRRC33) | * Modified human kappa light chain signal | |
| | peptide is depicted in bold font. | |
| | ** Histidine tag is underlined. | |
| Human LRRC33-GARP | MDMRVPAQLLGLLLLWFSGVLGWRNRSGTATAASQGVCKLVGGAAD | 1074 |
| chimera | CRGQSLASVPSSLPPHARMLTLDANPLKTLWNHSLQPYPLLESLSLHSC | |
| | HLERISRGAFQEQGHLRSLVLGDNCLSENYEETAAALHALPGLRRLDLS | |
| | GNALTEDMAALMLQNLSSLRSVSLAGNTIMRLDDSVFEGLERLRELDLQ | |
| | RNYIFEIEGGAFDGLAELRHLNLAFNNLPCIVDFGLTRLRVLNVSYNVLE | |
| | WFLATGGEAAFELETLDLSHNQLLFFPLLPQYSKLRTLLLRDNNMGFYR | |
| | DLYNTSSPREMVAQFLLVDGNVTNITTVSLWEEFSSSDLADLRFLDMSQ | |
| | NQFQYLPDGFLRKMPSLSHLNLHQNCLMTLHIREHEPPGALTELDLSHN | |
| | QLSELHLAPGLASCLGSLRLFNLSSNQLLGVPPGLFANARNITTLDMSH | |
| | NQISLCPLPAASDRVGPPSCVDFRNMASLRSLSLEGCGLGALPDCPFQ | |
| | GTSLTYLDLSSNWGVLNGSLAPLQDVAPMLQVLSLRNMGLHSSFMALD | |
| | FSGFGNLRDLDLSGNCLTTFPRFGGSLALETLDLRRNSLTALPQKAVSE | |
| | QLSRGLRTIYLSQNPYDCCGVDGWGALQHGQTVADWAMVTCNLSSKII | |
| | RVTELPGGVPRDCKWERLDLGLLIIILTFILVSAILLTTLAACCCVRRQKEN | |
| | QQYKA | |
| | * Modified human kappa light chain signal peptide is depicted in bold font. | |
| | ** LRRC33 ectodomain is underlined. | |
| | # GARP transmembrane domain is italicized. | |
| | ## GARP intracellular tail is double underlined. | |

### Pharmaceutical Compositions and Formulations

The disclosure further provides pharmaceutical compositions used as a medicament suitable for administration in human and non-human subjects. One or more high-affinity, context-independent antibodies encompassed by the disclosure can be formulated or admixed with a pharmaceutically acceptable carrier (excipient), including, for example, a buffer, to form a pharmaceutical composition. Such formulations may be used for the treatment of a disease or disorder that involves TGFβ signaling. In certain embodiments, such formulations may be used for immuno-oncology applications.

The pharmaceutical compositions of the disclosure may be administered to patients for alleviating a TGFβ-related indication (e.g., fibrosis, immune disorders, and/or cancer). “Acceptable” means that the carrier is compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. In one example, a pharmaceutical composition described herein contains more than one antibody that specifically binds a GARP-proTGFβ1 complex, a LTBP1-proTGFβ1 complex, a LTBP3-proTGFβ1 complex, and a LRRC33-proTGFβ1 complex where the antibodies recognize different epitopes/residues of the complex.

The pharmaceutical compositions to be used in the present methods can comprise pharmaceutically acceptable carriers, excipients, or stabilizers in the form of lyophilized formulations or aqueous solutions (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONIC® or polyethylene glycol (PEG). Pharmaceutically acceptable excipients are further described herein.

The disclosure also includes pharmaceutical compositions that comprise an antibody or fragment thereof according to the present disclosure, and a pharmaceutically acceptable excipient.

Thus, the antibody or a molecule comprising an antigen-binding fragment of such antibody can be formulated into a pharmaceutical composition suitable for human administration.

The pharmaceutical formulation may include one or more excipients. In some embodiments, excipient(s) may be selected from the list accessed at the following: accessdata.fda.gov/scripts/cder/iig/index.Cfm?event=browseByLetter.page&Letter=A

The pharmaceutical composition is typically formulated to a final concentration of the active biologic (e.g., monoclonal antibody, engineered binding molecule comprising an antigen-binding fragment, etc.) to be between about 20 mg/mL and about 200 mg/mL. For example, the final concentration (wt/vol) of the formulations may range between about 2-200, 2-180, 2-160, 2-150, 2-120, 2-100, 2-80, 2-70, 2-60, 2-50, 2-40, 5-200, 5-180, 5-160, 5-150, 5-120, 5-100, 5-80, 5-70, 5-60, 5-50, 5-40, 10-200, 10-180, 10-160, 10-150, 10-120, 10-100, 10-80, 10-70, 10-60, 10-50, 10-40, 20-200, 20-180, 20-160, 20-150, 20-120, 20-100, 20-80, 20-70, 20-60, 20-50, 20-40, 30-200, 30-180, 30-160, 30-150, 30-120, 30-100, 30-80, 30-70, 30-60, 30-50, 30-40, 40-200, 40-180, 40-160, 40-150, 40-120, 40-100, 40-80, 40-70, 40-60, 40-50, 50-200, 50-180, 50-160, 50-150, 50-120, 50-100, 50-80, 50-70, 50-60, 60-200, 60-180, 60-160, 60-150, 60-120, 60-100, 60-80, 60-70, 70-200, 70-180, 70-160, 70-150, 70-120, 70-100, 70-80 mg/mL. In some embodiments, the final concentration of the biologic in the formulation is about 10, 15, 20, 25, 30, 35, 40, 45, 50, 55, 60, 65, 70, 75, 80, 85, 90, 95, 100, 110, 120, 130, 140, 150, 160, 170, 180, 190, or 200 mg/mL.

The pharmaceutical compositions of the present disclosure are preferably formulated with suitable buffers. Suitable buffers include but are not limited to: phosphate buffer, citric buffer, and histidine buffer.

The final pH of the formulation is typically between pH 5.0 and 8.0. For example, the pH of the pharmaceutical composition may be about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7 or 7.8.

The pharmaceutical composition of the present disclosure may comprise a surfactant, such as nonionic detergent, approved for the use in pharmaceutical formulations. Such surfactants include, for example, polysorbates, such as Polysorbate 20 (Tween™-20), Polysorbate 80 (Tween-80) and NP-40.

The pharmaceutical composition of the present disclosure may comprise a stabilizer. For liquid-protein preparations, stability can be enhanced by selection of pH-buffering salts, and often amino acids can also be used. It is often interactions at the liquid/air interface or liquid/solid interface (with the packaging) that lead to aggregation following adsorption and unfolding of the protein. Suitable stabilizers include but are not limited to: sucrose, maltose, sorbitol, as well as certain amino acids such as histidine, glycine, methionine and arginine.

The pharmaceutical composition of the present disclosure may contain one or any combinations of the following excipients: Sodium Phosphate, Arginine, Sucrose, Sodium Chloride, Tromethamine, Mannitol, Benzyl Alcohol, Histidine, Sucrose, Polysorbate 80, Sodium Citrate, Glycine, Polysorbate 20, Trehalose, Poloxamer 188, Methionine, Trehalose, Hyaluronidase, Sodium Succinate, Potassium Phosphate, Disodium Edetate, Sodium Chloride, Potassium Chloride, Maltose, Histidine Acetate, Sorbitol, Pentetic Acid, Human Serum Albumin, Pentetic Acid.

In some embodiments, the pharmaceutical composition of the present disclosure may contain a preservative.

The pharmaceutical composition of the present disclosure is typically presented as a liquid or a lyophilized form. Typically, the products can be presented in vial (e.g., glass vial). Products available in syringes, pens, or autoinjectors may be presented as pre-filled liquids in these container/closure systems.

In some examples, the pharmaceutical composition described herein comprises liposomes containing an antibody that specifically binds a GARP-proTGFβ1 complex, a LTBP1-proTGFβ1 complex, a LTBP3-proTGFβ1 complex, and a LRRC33-proTGFβ1 complex, which can be prepared by any suitable method, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, liposomes with targeting properties are selected to preferentially deliver or localize the pharmaceutical composition to certain tissues or cell types. For example, certain nanoparticle-based carriers with bone marrow-targeting properties may be employed, e.g., lipid-based nanoparticles or liposomes. See, for example, Sou (2012) “Advanced drug carriers targeting bone marrow”, ResearchGate publication 232725109.

In some embodiments, pharmaceutical compositions of the disclosure may comprise or may be used in conjunction with an adjuvant. It is contemplated that certain adjuvant can boost the subject's immune responses to, for example, tumor antigens, and facilitate T effector function, DC differentiation from monocytes, enhanced antigen uptake and presentation by APCs, etc. Suitable adjuvants include but are not limited to retinoic acid-based adjuvants and derivatives thereof, oil-in-water emulsion-based adjuvants, such as MF59 and other squalene-containing adjuvants, Toll-like receptor (TRL) ligands (e.g., CpGs), α-tocopherol (vitamin E) and derivatives thereof.

The antibodies described herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™ Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody of the disclosure with Intralipid™ or the components thereof (soybean oil, egg phospholipids, glycerol and water).

### Kits for Use in Detecting, Monitoring or Alleviating a TGFβ-Related Indication

The present disclosure also provides kits for use in alleviating diseases/disorders associated with a TGFβ-related indication. Such kits can include one or more containers comprising an antibody, or antigen binding portion thereof, that specifically binds to a GARP-TGFβ1 complex, a LTBP1-TGFβ1 complex, a LTBP3-TGFβ1 complex, and/or a LRRC33-TGFβ1 complex, e.g., any of those described herein.

In some embodiments, the kit can comprise instructions for use in accordance with any of the methods described herein. The included instructions can comprise a description of administration of the antibody, or antigen binding portion thereof, that specifically binds a GARP-TGFβ1 complex, a LTBP1-TGFβ1 complex, a LTBP3-TGFβ1 complex, and/or a LRRC33-TGFβ1 complex to treat, delay the onset, or alleviate a target disease as those described herein. The kit may further comprise a description of selecting an individual suitable for treatment based on identifying whether that individual has the target disease. In still other embodiments, the instructions comprise a description of administering an antibody, or antigen binding portion thereof, to an individual at risk of the target disease.

The instructions relating to the use of antibodies, or antigen binding portions thereof, that specifically binds a GARP-TGFβ1 complex, a LTBP1-TGFβ1 complex, a LTBP3-TGFβ1 complex, and/or a LRRC33-TGFβ1 complex generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder associated with a TGFβ-related indication. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody, or antigen binding portion thereof, that specifically binds a GARP-TGFβ1 complex, a LTBP1-TGFβ1 complex, a LTBP3-TGFβ1 complex, and/or a LRRC33-TGFβ1 complex as those described herein.

Kits may optionally provide additional components such as buffers and interpretive information. Normally, the kit comprises a container and a label or package insert(s) on or associated with the container. In some embodiments, the disclosure provides articles of manufacture comprising contents of the kits described above.

### Modifications

Antibodies, or antigen binding portions thereof, of the disclosure may be modified with a detectable label or detectable moiety, including, but not limited to, an enzyme, prosthetic group, fluorescent material, luminescent material, bioluminescent material, radioactive material, positron emitting metal, nonradioactive paramagnetic metal ion, and affinity label for detection and isolation of a GARP-proTGFβ1 complex, a LTBP1-proTGFβ1 complex, a LTBP3-proTGFβ1 complex, and/or a LRRC33-proTGFβ1 complex. The detectable substance or moiety may be coupled or conjugated either directly to the polypeptides of the disclosure or indirectly, through an intermediate (such as, for example, a linker (e.g., a cleavable linker)) using suitable techniques. Non-limiting examples of suitable enzymes include horseradish peroxidase, alkaline phosphatase, β-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (131I, 125I, 123I, 121I), carbon (14C), sulfur (35S), tritium (3H), indium (115mln, 113mln, 112In, 111In), and technetium (99Tc, 99mTc), thallium (201Ti), gallium (68Ga, 67Ga), palladium (103Pd), molybdenum (99Mo), xenon (133Xe), fluorine (18F), 153Sm, Lu (177Lu), 159Gd, 149Pm, 140 La, 175Yb, 166Ho, 90Y, 47Sc, 86R, 188Re, 142Pr, 105Rh, 97Ru, 68Ge, 57Co, 65Zn, 85Sr, 32P, 153Gd, 169Yb, 51Cr, 54Mn, 75Se, Zirconium (⁸⁹Zr) and tin (113Sn, 117Sn). In some embodiments, the radio label may be selected from the group consisting of: ¹¹C, ¹³N, ¹⁵O, ⁶⁸Ga, ¹⁷⁷Lu, ¹⁸F and ⁸⁹Zr. In some embodiments, useful labels are positron-emitting isotopes, which may be detected by positron-emission tomography. The detectable substance may be coupled or conjugated either directly to the antibodies of the disclosure that bind specifically to a GARP-proTGFβ1 complex, a LTBP1-proTGFβ1 complex, a LTBP3-proTGFβ1 complex, and/or a LRRC33-proTGFβ1 complex, or indirectly, through an intermediate (such as, for example, a linker) using suitable techniques. Any of the antibodies provided herein that are conjugated to a detectable substance may be used for any suitable diagnostic assays, such as those described herein. Such assays include in vivo imaging, which may be used for monitoring disease progression and/or response to a therapy, such as TGFβ1 inhibition therapy described herein.

In addition, antibodies, or antigen binding portions thereof, of the disclosure may also be modified with a drug. The drug may be coupled or conjugated either directly to the polypeptides of the disclosure, or indirectly, through an intermediate (such as, for example, a linker (e.g., a cleavable linker)) using suitable techniques.

### Targeting Agents

In some embodiments methods of the present disclosure comprise the use of one or more targeting agents to target an antibody, or antigen binding portion thereof, as disclosed herein, to a particular site in a subject for purposes of modulating mature TGFβ release from a GARP-proTGFβ1 complex, a LTBP1-proTGFβ1 complex, a LTBP3-proTGFβ1 complex, and/or a LRRC33-proTGFβ1 complex. For example, LTBP1-proTGFβ1 and LTBP3-proTGFβ1 complexes are typically localized to extracellular matrix. Thus, in some embodiments, antibodies disclosed herein can be conjugated to extracellular matrix targeting agents for purposes of localizing the antibodies to sites where LTBP-associated TGFβ1 complexes reside. In such embodiments, selective targeting of antibodies leads to selective modulation of LTBP1-proTGFβ1 and LTBP3-proTGFβ1 complexes. In some embodiments, extracellular matrix targeting agents include heparin binding agents, matrix metalloproteinase binding agents, lysyl oxidase binding domains, fibrillin-binding agents, hyaluronic acid binding agents, and others.

Similarly, GARP-proTGFβ1 and LRRC33-proTGFβ1 complexes are typically localized and anchored to the surface of cells. The former is expressed on activated FOXP3+ regulatory T cells (Tregs), while the latter is expressed on certain myeloid cells and some cancer cells such as AML. Thus, in some embodiments, antibodies disclosed herein can be conjugated to immune cell (e.g., Treg cell, activated macrophages, etc.) binding agents for purposes of localizing antibodies to sites where these cell-associated proTGFβ1 complexes reside. In such embodiments, selective targeting of antibodies leads to selective inhibition of cell associated-proTGFβ1 complexes (e.g., selective inhibition of the release of mature TGFβ1 for purposes of immune modulation, e.g., in the treatment of cancer). In such embodiments, immune cell targeting agents may include, for example, CCL22 and CXCL12 proteins or fragments thereof.

In some embodiments, bispecific antibodies may be used having a first portion that selectively binds a proTGFβ1 complex and a second portion that selectively binds a component of a target site, e.g., a component of the ECM (e.g., fibrillin) or a component of a Treg cell (e.g., CTLA-4).

As further detailed herein, the present disclosure contemplates that isoform-selective TGFβ1 inhibitors, such as those described herein, may be used for promoting or restoring hematopoiesis in the bone marrow. Accordingly, in some embodiments, a composition comprising such an inhibitor (e.g., high-affinity, isoform-selective inhibitor of TGFβ1) may be targeted to the bone marrow. One mode of achieving bone marrow targeting is the use of certain carriers that preferentially target the bone marrow localization or accumulation. For example, certain nanoparticle-based carriers with bone marrow-targeting properties may be employed, e.g., lipid-based nanoparticles or liposomes. See, for example, Sou (2012) “Advanced drug carriers targeting bone marrow”, ResearchGate publication 232725109.

In some embodiments, targeting agents include immune-potentiators, such as adjuvants comprising squalene and/or α-tocopherol and adjuvants comprising a TLR ligand/agonist (such as TLR3 ligands/agonists). For example, squalene-containing adjuvant may preferentially target certain immune cells such as monocytes, macrophages and antigen-presenting cells to potentiate priming, antigen processing and/or immune cell differentiation to boost host immunity. In some embodiments, such adjuvant may stimulate host immune responses to neo-epitopes for T cell activation. GARP-proTGFβ1 as target

Regulatory T cells (Tregs) represent a small subset of CD4-positive T lymphocytes and play an important role of acting as a “break” in dampening immune responses to maintain homeostasis. In disease conditions (such as cancer), elevated levels of Tregs are reported, and this is associated with poorer prognosis. Human Tregs isolated from peripheral blood cells of donors can be activated by CD3/CD28 stimulation. Treg activation is shown to induce a marked increase in GARP-proTGFβ1 cell surface expression. As reported previously, Tregs exert immune suppressive activities, which include powerful suppression of effector T cell (Teff) proliferation. Under the standard experimental conditions where most Teffs undergo cell division, co-cultured Tregs reduce this to a mere fraction. And this Treg inhibition of Teff proliferation can be effectively overcome (i.e., disinhibition) by treating the co-culture of Teffs and Tregs with isoform-selective inhibitors of TGFβ1, demonstrating that isoform-selective TGFβ1 disclosed herein are effective in inhibiting the GARP-arm of TGFβ1 function. In disease environments (such as tumor microenvironment and fibrotic environment), this would translate to the ability of these inhibitors to block Treg-mediated immunosuppression. This should in turn lead to enhanced proliferation of effector T cells to boost immunity. The GARP-arm of the isoform-selective inhibitors of TGFβ1 may target this facet of TGFβ1 function. In some embodiments, the antibodies, or the antigen binding portions thereof, as described herein, may reduce the suppressive activity of regulatory T cells (Tregs).

### LRRC33-proTGFβ1 as Target

LRRC33 is expressed in selective cell types, in particular those of myeloid lineage, including monocytes and macrophages. Monocytes originated from progenitors in the bone marrow and circulate in the bloodstream and reach peripheral tissues. Circulating monocytes can then migrate into tissues where they become exposed to the local environment (e.g., tissue-specific, disease-associated, etc.) that includes a panel of various factors, such as cytokines and chemokines, triggering differentiation of monocytes into macrophages, dendritic cells, etc. These include, for example, alveolar macrophages in the lung, osteoclasts in bone marrow, microglia in the CNS, histiocytes in connective tissues, Kupffer cells in the liver, and brown adipose tissue macrophages in brown adipose tissues. In a solid tumor, infiltrated macrophages may be tumor-associated macrophages (TAMs), tumor-associated neutrophils (TANs), and myeloid-derived suppressor cells (MDSCs), etc. Such macrophages may activate and/or be associated with activated fibroblasts, such as carcinoma-associated (or cancer-associated) fibroblasts (CAFs) and/or the stroma. Thus, inhibitors of TGFβ1 activation described herein which inhibits release of mature TGFβ1 from LRRC33-containing complexes can target any of these cells expressing LRRC33-proTGFβ1 on cell surface. At a fibrotic microenvironment, LRRC33-expressing cells may include M2 macropahges, tissue resident macrophages, and/or MDSCs.

In some embodiments, the LRRC33-TGFβ1 complex is present at the outer surface of profibrotic (M2-like) macrophages. In some embodiments, the profibrotic (M2-like) macrophages are present in the fibrotic microenvironment. In some embodiments, targeting of the LRRC33-TGFβ1 complex at the outer surface of profibrotic (M2-like) macrophages provides a superior effect as compared to solely targeting LTBP1-TGFβ1 and/or LTBP1-TGFβ1 complexes. In some embodiments, M2-like macrophages, are further polarized into multiple subtypes with differential phenotypes, such as M2c and M2d TAM-like macrophages. In some embodiments, macrophages may become activated by various factors (e.g., growth factors, chemokines, cytokines and ECM-remodeling molecules) present in the tumor microenvironment, including but are not limited to TGFβ1, CCL2 (MCP-1), CCL22, SDF-1/CXCL12, M-CSF (CSF-1), IL-6, IL-8, IL-10, IL-11, CXCR4, VEGF, PDGF, prostaglandin-regulating agents such as arachidonic acid and cyclooxygenase-2 (COX-2), parathyroid hormone-related protein (PTHrP), RUNX2, HIF1α, and metalloproteinases. Exposures to one or more of such factors may further drive monocytes/macrophages into pro-tumor phenotypes. To give but one example, CCL2 and VEGF co-expression in tumors has been shown to be correlated with increased TAM and poor diagnosis. In turn, activated tumor-associated cells may also facilitate recruitment and/or differentiation of other cells into pro-tumor cells, e.g., CAFs, TANs, MDSCs, and the like. Stromal cells may also respond to macrophage activation and affect ECM remodeling, and ultimately vascularization, invasion, and metastasis. For example, CCL2 not only functions as a monocyte attractant but also promotes cell adhesion by upregulating MAC-1, which is a receptor for ICAM-1, expressed in activated endothelium. This may lead to CCL2-dependent arteriogenesis and cancer progression. Thus, TGFβ1 inhibitors described herein may be used in a method for inhibiting arteriogenesis by interfering with the CCL2 signaling axis.

A subset of myeloid cells express cell surface LRRC33, including M2-polarized macrophages and myeloid-derived suppressor cells (MDSCs), both of which have immunosuppressive phenotypes and are enriched at disease environments (e.g., TME and FME). Bone marrow-derived circulating monocytes do not appear to express cell surface LRRC33. The restrictive expression of LRRC33 makes this a particularly appealing therapeutic target. While a majority of studies available in the literature have focused on effector T cell biology (e.g., CD8+ cytotoxic cells) in cancer, increasing evidence (such as data presented herein) points to important roles of suppressive myeloid cell populations in diseases. Importantly, the highly selective TGFβ1 inhibitory antibodies disclosed herein, are capable of targeting this arm of TGFβ1 function in vivo. More specifically, data presented herein show that tumor-associated M2 macrophages and MDSCs express cell-surface LRRC33, with a strong correlation to disease progression. The high-affinity, TGFβ1-selective antibodies disclosed herein are capable of overcoming primary resistance to checkpoint blockade therapy (CBT) of tumors in multiple pharmacological models. Indeed, anti-tumor efficacy coincides with a significant decrease in tumor-associated macrophages and MDSC levels, suggesting that targeting this facet of TGFβ1 function may contribute to therapeutically beneficial effects. This is likely applicable to other disease where these immunosuppressive cells are enriched. A number of fibrotic conditions are also associated with elevated local frequencies of these cell populations. Thus, the high-affinity, TGFβ1-selective antibodies are expected to exert similar in vivo effects in such indications. Notably, the newfindings provided herein identify LRRC33 as a novel cell surface marker for MDSCs in circulation, this, coupled with Applicant's previous findings that i) the degree of tumor burden correlates with tumor associated MDSCs, and that ii) tumor-associated MDSCs correlate with circulatory MDSCs, points to a new use for LRRC33 as a blood-based biomarker, e.g., indicative of immune suppression. As described herein, an immune-suppressed tumor may be particularly responsive to treatment comprising a TGFβ inhibitor (e.g., TGFβ1-selective inhibitor, e.g., Ab6) and optionally in combination with a checkpoint inhibitor therapy.

### LTBP1/3-proTGFβ1 as Target

The extracellular matrix is the site at which complex signaling events at the cellular, tissue, organ, and systemic levels are orchestrated. Dysregulation of the ECM is observed in a number of pathologies. A reservoir of TGFβ1 growth factor is present in the ECM in the form of latent proTGFβ1 complex. Latent proTGFβ1 complexes are anchored to the matrix via covalent interactions with the ECM components, LTBP1 and/or LTBP3. Other ECM proteins such as fibronectin and fibrillins (e.g., fibrillin-1) are believed to be important in mediating ECM deposition and localization of LTBPs. Targeting of LLCs to the ECM is an essential step in the TGFβ1 activation process. Because most, if not all, TGFβ1-related indications likely involve some aspects of ECM function that are TGFβ1-dependent, it is imperative that TGFβ inhibitors considered for therapeutics should be capable of targeting this pool of TGFβ1 signaling. Indeed, the high-affinity, isoform-selective inhibitors of TGFβ1 according to the present disclosure show remarkably high affinities and potency for human LTBP1/3-proTGFβ1 complexes. Because these antibodies directly target the ECM-localized complexes in their pre-activation state, this mechanism of action would do away with having to compete with endogenous high-affinity receptors for ligand binding. Further, because the inhibitory activities of these antibodies are localized at the site of disease associated with increased TGFβ1 activation (e.g., dysregulated niche within the ECM), it is envisaged that these antibodies should achieve enhanced efficacy while limiting side effects.

Cancer-associated fibroblasts (CAFs) are the primary contributors to the EMT signatures in the TME. In carcinoma, the tumor stroma comprises CAFs and contributes to excess ECM deposition, which can result in greater stiffness of the matrix. Applicant previously showed that increased stiffness of the matrix lowers the threshold of TGFβ1 activation. Tumors of such phenotype therefore are likely to benefit from TGFβ1 inhibitors.

In some embodiments, the LTBP1-TGFβ1 complex or the LTBP3-TGFβ1 complex is a component of the extracellular matrix. The N-terminus of LTBPs may be covalently bound to the ECM via an isopeptide bond, the formation of which may be catalyzed by transglutaminases. The structural integrity of the ECM is believed to be important in mediating LTBP-associated TGFβ1 activity. For example, stiffness of the matrix can significantly affect TGFβ1 activation. In addition, incorporating fibronectin and/or fibrillin in the scaffold may significantly increase the LTBP-mediated TGFβ1 activation. Similarly, presence of fibronectin and/or fibrillin in LTBP assays (e.g., cell-based potency assays) may increase an assay window. In some embodiments, the extracellular matrix comprises fibrillin and/or fibronectin. In some embodiments, the extracellular matrix comprises a protein comprising an RGD motif.

Thus, the high-affinity, isoform-selective inhibitors of TGFβ1 provided herein enable potent inhibition of each of the biological contexts of TGFβ1 function, namely, the GARP-arm, the LRRC33-arm, and the LTBP1/3-arm.

### TGFβ1-Related Indications

### General Features of TGFβ1-Related Indications

TGFβ1 inhibitors, such as isoform-selective inhibitors described herein, may be used to treat a wide variety of diseases, disorders and/or conditions that are associated with TGFβ1 dysregulation (i.e., “TGF #61-related indications”) in human subjects. As used herein, “disease (disorder or condition) associated with TGFβ1 dysregulation” or “TGFβ1-related indication” means any disease, disorder and/or condition related to expression, activity and/or metabolism of a TGFβ1 or any disease, disorder and/or condition that may benefit from inhibition of the activity and/or levels TGFβ1. A plethora of evidence exists in literature pointing to the dysregulation of the TGFβ signaling pathway in pathologies such as cancer and fibrosis.

Based on the inventors' recognition that TGFβ1 appears to be the predominant disease-associated isoform, the present disclosure includes the use of an isoform-selective, context-independent TGFβ1 inhibitor in a method for treating a TGFβ1-related indication in a human subject. Such inhibitor is typically formulated into a pharmaceutical composition that further comprises a pharmaceutically acceptable excipient. Advantageously, the inhibitor targets both ECM-associated TGFβ1 and immune cell-associated TGFβ1 but does not target TGFβ2 or TGFβ3 in vivo. In some embodiments, the inhibitor inhibits the activation step of TGFβ1. The disease may be characterized by dysregulation or impairment in at least two of the following attributes: a) regulatory T cells (Treg); b) effector T cell (Teff) proliferation or function; c) myeloid cell proliferation or differentiation; d) monocyte recruitment or differentiation; e) macrophage function; f) epithelial-to-mesenchymal transition (EMT) and/or endothelial-to-mesenchymal transition (EndMT); g) gene expression in one or more of marker genes selected from the group consisting of: PAI-1, ACTA2, CCL2, Col1a1, Col3a1, FN-1, CTGF, and TGFB1; h) ECM components or function; i) fibroblast differentiation. A therapeutically effective amount of such inhibitor is administered to the subject suffering from or diagnosed with the disease.

In some embodiments, such therapeutic use incorporates the step of diagnosing and/or monitoring treatment response as detailed herein. For example, circulating MDSCs and/or circulating latent. TGFβ1 may be used as biomarker, in accordance with the present disclosure. Such therapeutic use may further include a step of selecting a suitable TGFβ inhibitor as therapy and/or selecting a patient or patient population likely to benefit from such therapy.

In some embodiments, a disease treated herein may involve dysregulation or impairment of ECM components or function comprises that show increased collagen I deposition. In some embodiments, the dysregulation of the ECM includes increased stiffness of the matrix. In some embodiments, the dysregulation of the ECM involves fibronectin and/or fibrillin.

In some embodiments, the dysregulation or impairment of fibroblast differentiation comprises increased myofibroblasts or myofibroblast-like cells. In some embodiments, the myofibroblasts or myofibroblast-like cells are cancer-associated fibroblasts (CAFs). In some embodiments, the CAFs are associated with a tumor stroma and may produce CCL2/MCP-1 and/or CXCL12/SDF-1. In some embodiments, the myofibroblasts or myofibroblast-like cells are localized to a fibrotic tissue.

In some embodiments, the dysregulation or impairment of regulatory T cells comprises increased Treg activity.

In some embodiments, the dysregulation or impairment of effector T cell (Teff) proliferation or function comprises suppressed CD4+/CD8+ cell proliferation.

In some embodiments, the dysregulation or impairment of myeloid cell proliferation or differentiation comprises increased proliferation of myeloid progenitor cells. The increased proliferation of myeloid cells may occur in a bone marrow,

In some embodiments, the dysregulation or impairment of monocyte differentiation comprises increased differentiation of bone marrow-derived and/or tissue resident monocytes into macrophages at a disease site, such as a fibrotic tissue and/or a solid tumor.

In some embodiments, the dysregulation or impairment of monocyte recruitment comprises increased bone marrow-derived monocyte recruitment into a disease site such as TME, leading to increased macrophage differentiation and M2 polarization, followed by increased TAMs.

In some embodiments, the dysregulation or impairment of macrophage function comprises increased polarization of the macrophages into M2 phenotypes.

In some embodiments, the dysregulation or impairment of myeloid cell proliferation or differentiation comprises an increased number of Tregs, MDSCs and/or TANs.

TGFβ-related indications may include conditions comprising an immune-excluded disease microenvironment, such as tumor or cancerous tissue that suppresses the body's normal defense mechanism/immunity in part by excluding effector immune cells (e.g., CD4+ and/or CD8+ T cells). In some embodiments, such immune-excluding conditions are associated with poor responsiveness to treatment (e.g., cancer therapy). TGFβ-related indications also include conditions that do not comprise an immune-excluded disease microenvironment, such as an immune-infiltrated tumor or cancerous tissue. In some embodiments, these immune-infiltrated conditions are also associated with poor responsiveness to treatment (e.g., cancer therapy). Additionally, TGFβ-related indications include conditions that comprise an immune-desert disease microenvironment, which can also be associated with poor responsiveness to treatment (e.g., cancer therapy). Non-limiting examples of the cancer therapies, to which patients are poorly responsive, include but are not limited to: checkpoint inhibitor therapy, cancer vaccines, chemotherapy, and radiation therapy (such as a radiotherapeutic agent). Without intending to be bound by particular theory, it is contemplated that TGFβ inhibitors, such as those described herein, may help counter the tumor's ability to evade or exclude anti-cancer immunity by restoring immune cell access, e.g., T cell (e.g., CD8+ cells) and macrophage (e.g., F4/80+ cells, M1-polarized macrophages) access by promoting T cell expansion and/or infiltration into tumor, and/or by reducing the levels of immunosuppressive cells, such as MDSCs (e.g., gMDSCs) or Tregs.

Thus, TGFβ inhibition may overcome treatment resistance (e.g., immune checkpoint resistance, cancer vaccine resistance, CAR-T resistance, chemotherapy resistance, radiation therapy resistance (such as resistance to a radiotherapeutic agent), etc.) in an immune-excluded disease environment (such as TME) by unblocking and restoring effector T cell access and cytotoxic effector functions Alternatively or additionally, TGFβ inhibition may overcome treatment resistance (e.g., immune checkpoint resistance, cancer vaccine resistance, CAR-T resistance, chemotherapy resistance, radiation therapy resistance (such as resistance to a radiotherapeutic agent), etc.) in an immune-infiltrated disease environment (such as TME) by relieving the immunosuppressive effects of immunosuppressive cells (e.g., MDSCs, macrophages, and/or Tregs). Alternatively or additionally, TGFβ inhibition may overcome treatment resistance (e.g., immune checkpoint resistance, cancer vaccine resistance, CAR-T resistance, chemotherapy resistance, radiation therapy resistance (such as resistance to a radiotherapeutic agent), etc.) in an immune-desert disease environment (such as TME) by relieving the immunosuppressive effects of immunosuppressive cells (e.g., MDSCs, macrophages, and/or Tregs).Such effects of TGFβ inhibition may further provide long-lasting immunological memory mediated, for example, by CD8+ T cells.

In some embodiments, the tumor is poorly immunogenic (e.g., “desert” or “cold” tumors). In some embodiments, the tumor is immunogenic (e.g., “hot” tumors), but exhibits an immunosuppressive phenotype. Patients may benefit from cancer therapy that triggers neo-antigens or promote immune responses. Such therapies include, but are not limited to, chemotherapy, radiation therapy (such as a radiotherapeutic agent), oncolytic viral therapy, oncolytic peptides, tyrosine kinase inhibitors, neo-epitope vaccines, anti-CTLA4, instability inducers, DDR agents, NK cell activators, and various adjuvants such as TLR ligands/agonists. TGFβ1 inhibitors, such as those described herein, can be used in conjunction to boost the effects of cancer therapies. One mode of action for TGFβ1 inhibitors may be to normalize or restore MHC expression, thereby promoting T cell immunity.

Moreover, recent observations in murine fibrosis models suggest detrimental effects of TGFβ3 inhibition in tissues with dysregulated ECM, raising the possibility that role of TGFβ3 expands beyond homeostasis. Ample evidence suggests that dysregulation of the ECM is found in a number of disease conditions, including fibrosis and cancer. Indeed, many of the key profibrotic genes are also recognized among markers of various cancers. These markers include, for example, col1A1, col3A1, PAI-1, CCL2, ACTA2, FN-1, CTGF and TGFB1. Therefore, the finding that concurrent blockade of TGFβ3 appears harmful in fibrosis may be applicable to a broader scope of conditions associated with ECM dysregulation.

In addition to the possible concerns of inhibiting TGFβ3 addressed above, Takahashi et al., (Nat Metab. 2019, 1(2): 291-303) recently reported a beneficial role of TGFβ2 in regulating metabolism. The authors identified TGFβ2 as an exercise-induced adipokine, which stimulated glucose and fatty acid uptake in vitro, as well as tissue glucose uptake in vivo; which improved metabolism in obese mice; and, which reduced high fat diet-induced inflammation. Moreover, the authors observed that lactate, a metabolite released from muscle during exercise, stimulated TGFβ2 expression in human adipocytes and that a lactate-lowering agent reduced circulating TGFβ2 levels and reduced exercise-stimulated improvements in glucose tolerance. These observations suggest that therapeutic use of a TGFβ inhibitor with inhibitory activity towards the TGFβ2 isoform may be harmful, at least in the metabolic aspect.

In some embodiments, the TGFβ inhibitor is TGFβ1-selective in that it does not inhibit TGFβ2. In some embodiments, the TGFβ inhibitor is TGFβ1-selective in that it does not inhibit TGFβ3. In preferred embodiments, the TGFβ inhibitor is TGFβ1-selective in that it does not inhibit TGFβ2 and TGFβ3.

Related methods for selecting a TGFβ inhibitor for therapeutic use are also encompassed herein. According to some embodiments, the TGFβ inhibitor is TGFβ-1 selective.

In some embodiments, the disease is characterized by dysregulation or impairment of myeloid cell proliferation or differentiation; wherein optionally the dysregulation or impairment of myeloid cells comprises monocyte recruitment to the disease site or differentiation into polarized M2 cells, and/or, abnormal macrophage function. In some embodiments, the dysregulation of myeloid cells comprises increased levels of MDSCs. Elevated MDSCs may comprise an increased number/frequency of circulating MDSCs, e.g., in peripheral blood. Elevated MDSCs may be observed at the site of the disease, such as fibrotic tissues and solid tumors. The terms circulating and circulatory (as in “circulating MDSCs” and “circulatory MDSCs”) may be used interchangeably.

In some embodiments, the disease is characterized by abnormal cell differentiation involving epithelial-to-mesenchymal transition (EMT) and/or endothelial-to-mesenchymal transition (EndMT). In some embodiments, these processes occurring at the disease sites (such as TME and fibrotic microenvironment) result in increased myofibroblasts or myofibroblast-like cells at the site. These include, for example, CAFs and FAFs. In some embodiments, the disease involving epithelial-to-mesenchymal transition (EMT) is ccRCC.

In some embodiments, the disease is characterized by abnormal gene expression in one or more of marker genes selected from the group consisting of: SERPINE/PAI-1, ACTA2, CCL2, Col1a1, Col3a1, FN-1, CTGF, and TGFB1, TGFB2, TGFB3, LTBP1, LTBP3, LRRC32/GARP, LRRC33, MMP2, MMP9, LOXL2, THSB1, TIMP1.

Involvement of the TGFβ1 pathway in controlling key facets of both the ECM and immune components may explain the observations that a remarkable number of dysregulated genes are shared across a wide range of pathologies such as proliferative disorders and fibrotic disorders. This supports the notion that the aberrant pattern of expression in the genes involving TGFβ1 signaling is likely a generalizable phenomenon. These marker genes may be classified into several categories such as: genes involved in mesenchymal transition (e.g., EndMT and EMT); genes involved in angiogenesis; genes involved in hypoxia; genes involved in wound healing; and genes involved in tissue injury-triggered inflammatory response.

A comprehensive study carried out by Hugo et al., (Cell, 165(1): 35-44) elegantly demonstrated the correlation between differential gene expression patterns of these classes of markers and the responsiveness to checkpoint blockade therapy (CBT) in metastatic melanoma. The authors found co-enrichment of the set of genes coined “IPRES signatures” defined a transcriptomic subset within not only melanoma, but also all major common human malignancies analyzed. Indeed, the work links tumor cell phenotypic plasticity (i.e., mesenchymal transition) and the resultant impacts on the microenvironment (e.g., ECM remodeling, cell adhesion, and angiogenesis features of immune suppressive wound healing) to CBT resistance. In addition to IPRES, other gene signatures such as TIDE (Jing et al., Nat Med. 2018 October; 24(10):1550-1558), TIS (Danaher et al., J Immunother Cancer. 2018 Jun. 22; 6(1):63), F-TBRS (Mariathasan et al., Nature. 2018 Feb. 22; 554(7693): 544-548), IMPRES (Auslander et al., Nat Med. 2018 October; 24(10): 1545-1549), and xCell (Aran et al. Genome Biol. 2017 Nov. 15; 18(1):220) may also be used to evaluate the tumor immune microenvironment.

Recognizing that each of these IPRES gene categories has been implicated in disease involving TGFβdysregulation, Applicant previously contemplated that the TGFβ1 isoform in particular may mediate these processes in disease conditions (see, for example, WO 2017/156500).

Accordingly, the present disclosure includes a method/process of selecting or identifying a candidate patient or patient population likely to respond to a TGFβ1 inhibition therapy, and administering to the patient(s) an effective amount of a high-affinity isoform-selective inhibitor of TGFβ1. Observation of a patient's lack of responsiveness to a CBT (e.g., resistance) may indicate that the patient is a candidate for the TGFβ1 inhibition therapy described herein. Thus, an isoform-selective inhibitor of TGFβ1 such as Ab6 may be used in the treatment of cancer in a subject, wherein the subject is poorly responsive to a CBT. The subject may have advanced cancer, such as a locally advanced solid tumor or metastatic cancer. A patient is said to be “poorly responsive” when there is no or little meaningful therapeutic effects achieved (e.g., do not meet the criteria of partial response or compete response based on standard guidelines, such as RECIST and iRECIST) following a duration of time which is expected to be sufficient to show meaningful therapeutic effects of the particular therapy. Typically, such duration of time for CBTs is at least about 3 months of treatment, either with or without additional therapies such as chemotherapy. Such patients may be referred to as “refractory” or “non-responders.” Where such patients are poorly responsive to the initial CBT, the patients may be referred to as “primary non-responders.” Cancer (or patients with such cancer) in this category may be characterized as having “primary resistance” to the CBT. In some embodiments, the subject is a primary non-responder after receiving at least about 3 months of the CBT treatment, wherein optionally, after at least about 4 months of the CBT treatment. In some embodiments, the subject also received additional therapy in combination with the CBT, such as chemotherapy.

Upon identification of the subject as a non-responder of a CBT, the high-affinity, isoform-selective inhibitor of TGFβ1 may be administered to the subject in conjunction with a CBT, which may or may not comprise the same checkpoint inhibitor as the first CBT to which the subject failed to respond. Any suitable immune checkpoint inhibitors may be used, e.g., approved checkpoint inhibitors. In some embodiments, the high-affinity, isoform-selective inhibitor of TGFβ1 is administered to the subject in conjunction with a CBT comprising an anti-PD-1 antibody or anti-PD-L1 antibody. The high-affinity, isoform-selective inhibitor of TGFβ1 is aimed to overcome the resistance by rendering the cancer more susceptible to the CBT.

The process of selecting or identifying a candidate patient or patient population likely to respond to, or otherwise likely to benefit from, a TGFβ1 inhibition therapy may comprise a step of testing a biological sample collected from the patient (or patient population), such as biopsy samples, for the expression of one or more of the markers discussed herein. Similarly, such genetic marker(s) may be used for purposes of monitoring the patient's responsiveness to a therapy. Monitoring may include testing two or more biological samples collected from the patient, for example, before and after administration of a therapy, and during the course of a therapeutic regimen over time, to evaluate changes in gene expression levels of one or more of the markers, indicative of therapeutic response or effectiveness. In some embodiments, a liquid biopsy may be used.

In some embodiments, a method of selecting a candidate patient or patient population likely to respond to a TGFβ1 inhibition therapy may comprise a step of identifying a patient or patient population previously tested for the genetic marker(s), such as those described herein, which showed aberrant expression thereof. These same methods are also applicable to later confirming or correlating with the patients' response to the therapy.

In some embodiments, the aberrant marker expression includes elevated levels of at least one of the following: TGFβ1, LRRC33, GARP, LTBP1, LTBP3, CCL2, CCL3, PAI-1/Serpine1, MMP2, MMP9, Col1a1, Col3a1, FN1, CTGF, α-SMA, ITGA11, and ACTA2. In some embodiments, the abberant marker includes elevated levels of at least one of the following: MMP2, Col1a1, Col3a1, Fn1, CTGF, TIMP1, TGFB1, TGFB2, TGFB3, LTBP1, LTBP3, LRRC33, LRRC32/GARP, SERPINE1/PAI-1, THBS1, and Loxl2. In some embodiments, the patient or patient population (e.g., biological samples collected therefrom) shows elevated TGFβ1 activation, phospho-Smad2, phospho-Smad2/3, or combination thereof. In some embodiments, the patient or patient population shows elevated BUN. In some embodiments, the patient or patient population (e.g., biological samples collected therefrom) shows elevated MDSCs. In some embodiments, such patient or patient population has cancer, which may comprise a solid tumor that is TGFβ1-positive. The solid tumor may be a TGFβ1-dominant tumor, in which TGFβ1 is the predominant isoform expressed in the tumor, relative to the other isoforms. In some embodiments, the solid tumor may be a TGFβ1-co-dominant tumor, in which TGFβ1 is the co-dominant isoform expressed in the tumor, e.g., TGFβ1+/TGFβ3+. In some embodiments, such patient or patient population exhibits resistance to a cancer therapy, such as chemotherapy, radiation therapy (such as a radiotherapeutic agent) and/or immune checkpoint therapy, e.g., anti-PD-1 (e.g., pembrolizumab, budigalimab, nivolumab, spartalizumab), anti-PD-L1 (e.g., atezolizumab), anti-CTLA4 (e.g., ipilimumab), engineered immune cell therapy (e.g., CAR-T), and cancer vaccines, etc. According to the disclosure, TGFβ1 inhibitors provided herein, such as Ab6, overcome the resistance by unblocking immunosuppression so as to allow effector cells to gain access to cancer cells thereby achieving anti-tumor effects. TGFβ1 inhibitor therapy may therefore promote effector cell infiltration and/or expansion in the tumor. Additionally, TGFβ1 inhibitor therapy may reduce the frequency of immunosuppressive immune cells, such as Tregs and MDSCs, in the tumor. In some embodiments, the patient or patient population has a fibrotic disorder or disease (such as organ fibrosis, in particular lung fibrosis).

In some embodiments, the aberrant marker expression includes one or more panels of genes: mesenchymal transition markers (e.g., AXL, ROR2, WNT5A, LOXL2, TWIST2, TAGLN, FAP); immunosuppressive genes (e.g., IL10, VEGFA, VEGFC); monocyte and macrophage chemotactic genes (e.g., CCL2, CCL7, CCL8, CCL13); genes involved in angiogenesis and wound healing (e.g., T cell suppressive); cell adhesion markers; ECM remodeling; skeletal system and bone development markers; and genes involved in tissue injury-triggered inflammatory response.

In some embodiments, lack or downregulation of MHC expression (such as MHC class 1) may serve as a biomarker for TGFβ1-associated conditions for which the antibodies or antigen-binding fragments encompassed by the present disclosure may be used as therapy. Reduced MHC levels may signal immune escape, which may correlate with poor responsiveness of the patients to immune therapies, such as CBT. Selective inhibition of TGFβ1 therefore may at least in part restore effector cell function.

The present disclosure provides a TGFβ inhibitor (e.g., TGFβ1-selective inhibitor such as Ab6) for use in the treatment of a TGFβ-related disorder with aberrant gene expression (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

In some embodiments, a method of selecting a candidate patient or patient population likely to respond to a TGFβ1 inhibition therapy for fibrosis may comprise a step of identifying a candidate patient, or patient population, who has received a background therapy or is on a background therapy for a fibrotic condition, for example, an approved standard of care therapy or regimen for the fibrotic condition.

In some embodiments, a method of selecting a candidate patient or patient population likely to respond to a TGFβ1 inhibition therapy for lung fibrosis may comprise a step of identifying a candidate patient, or patient population, who has received a background therapy or is on a background therapy for lung fibrosis, for example, an approved standard of care therapy or regimen for lung fibrosis.

### Circulating/Circulatory MDSCs as a Biomarker

In some embodiments, the patient or patient population (e.g., biological samples collected therefrom) shows elevated MDSCs. In some embodiments, the sample is a whole blood sample or a blood component (e.g., plasma or serum). In some embodiments, the sample is fresh whole blood or a blood component (e.g., of a sample that has not been previously frozen).

In certain embodiments, a TGFβ inhibitor described herein, e.g., an isoform-selective activation inhibitor of TGFβ1 such as Ab2, Ab46, Ab50, or derivatives thereof, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) is administered such that the amount (e.g., dose) of TGFβ1 inhibition administered is sufficient to reduce circulating MDSC levels by at least 10%, at least 15%, at least 20%, at least 25%, or more, as compared to baseline MDSC levels. Circulating MDSC levels may be measured prior to or after each treatment or each dose of the TGFβ inhibitor such that a decrease of at least 10%, at least 15%, at least 20%, at least 25%, or more in circulating MDSC levels may be indicative or predictive of treatment efficacy. In some embodiments, the level of circulating MDSCs may be used to determine disease burden (e.g., as measured by a change in fibrosis before and after a treatment regimen). In certain embodiments, a decrease in circulating MDSC levels may be indicative of a decrease in disease burden (e.g., a decrease in fibrosis). For instance, circulating MDSC levels may be measured prior to and after the administration of a dose of a TGFβ inhibitor described herein, e.g., an isoform-selective activation inhibitor of TGFβ1 such as Ab2, Ab46, Ab50, or derivatives thereof, isoform-non-selective TGFβ inhibitors, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) and a reduction in circulating MDSC levels may be indicative or predictive of pharmacological effects, e.g., of a reduction in disease burden (e.g., a reduction in fibrosis). In certain embodiments, circulating MDSC levels may be measured prior to and following administration of a first dose of a TGFβ inhibitor, such as a TGFβ inhibitor described herein, e.g., an isoform-selective activation inhibitor of TGFβ1 such as Ab2, Ab46, Ab50, or derivatives thereof, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). In some embodiments, reduction in circulating MDSC levels is indicative or predictive of pharmacological effects and further warrants administration of a second or more dose(s) of the TGFβ inhibitor. In some embodiments, the first dose of the TGFβ inhibitor is the very first dose of TGFβ inhibitor received by the patient. In some embodiments, the first dose of the TGFβ inhibitor is the first dose of a given treatment regimen comprising more than one dose of TGFβ inhibitor. In another embodiment, circulating MDSC levels may be measured prior to and after combination treatment comprising a TGFβ inhibitor described herein, e.g., an isoform-selective activation inhibitor of TGFβ1 such as Ab2, Ab46, Ab50, or derivatives thereof. In some embodiments, the reduction of circulating MDSC levels following the treatment of a TGFβ inhibitor described herein, e.g., an isoform-selective activation inhibitor of TGFβ1 such as Ab2, Ab46, Ab50, or derivatives thereof, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3), may warrant continuation of treatment.

In some aspects, the disclosure provides a method of treating fibrosis (e.g., pulmonary fibrosis) in a subject, the method comprising steps of selecting a TGFβ inhibitor that inhibits TGFβ1 but does not inhibit one or both of TGFβ2 and TGFβ3; administering to a subject having a fibrotic condition the TGFβ inhibitor in an amount sufficient to reduce circulating MDSC levels. In some embodiments, the circulating MDSC levels are determined from whole blood or a blood component collected from the subject. In some embodiments, the circulating MDSC levels are reduced by at least 10%, optionally by at least 15%, 20%, 25%, or more.

In some embodiments, the isoform-selective inhibitor of TGFβ1 as described herein is used in a method of reducing circulating MDSC levels in a subject. In some embodiments, the circulating MDSC levels are determined from whole blood or a blood component collected from the subject. In some embodiments, the circulating MDSC levels are reduced by at least 10%, optionally by at least 15%, 20%, 25%, or more. In some embodiments, the TGFβ inhibitor is administered in an amount sufficient to reduce circulating MDSC levels.

In certain embodiments of the present disclosure, levels of circulating MDSCs may be used to predict, determine, and monitor pharmacological effects of treatment comprising a dose of TGFβ inhibitor, such as a TGFβinhibitor described herein, e.g., an isoform-selective activation inhibitor of TGFβ1 such as Ab2, Ab46, Ab50, or derivatives thereof, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) administered alone or in conjunction with another therapy. In certain embodiments, circulating MDSCs may be measured within six weeks following administration of the initial treatment (e.g., the (first) dose of TGFβ inhibitor). In certain embodiments, circulating MDSC levels may be measured within thirty days following administration of the initial dose of TGFβ inhibitor. In some embodiments, MDSC levels may be measured within or at about three weeks following administration of the initial dose of TGFβ inhibitor. In some embodiments, MDSC levels may be measured within or at about two weeks following administration of the initial dose of TGFβ inhibitor. In some embodiments, MDSC levels may be measured within or at about ten days following administration of the initial dose of TGFβ inhibitor.

### Diseases Involving Mesenchymal Transition

Mesenchymal transition is a process of phenotypic shift of cells, such as epithelial cells and endothelial cells, towards a mesenchymal phenotype (such as myofibroblasts). Examples of genetic markers indicative of mesenchymal transition include AXL, ROR2, WNT5, LOXL2, TWIST2, TAGLN and FAP. In cancer, for example, mesenchymal transition (e.g., increased EndMT and EMT signatures) indicates tumor cell phenotypic plasticity. Thus, inhibitors of TGFβ, e.g., TGFβ1 inhibitors, such as Ab6, may be used to treat a disease that is initiated or driven by mesenchymal transition, such as EMT and EndMT.

EMT (epithelial-to-mesenchymal transition) is the process by which epithelial cells with tight junctions switch to mesenchymal properties (phenotypes) such as loose cell-cell contacts. The process is observed in a number of normal biological processes as well as pathological situations, including embryogenesis, wound healing, cancer metastasis and fibrosis (reviewed in, for example, Shiga et al., (2015) “Cancer-Associated Fibroblasts: Their Characteristics and Their Roles in Tumor Growth.” Cancers, 7: 2443-2458). Generally, it is believed that EMT signals are induced mainly by TGFβ. Many types of cancer, for example, appear to involve transdifferentiation of cells towards mesenchymal phenotype (such as myofibroblasts and CAFs) which correlate with poorer prognosis. Thus, isoform-specific, context-independent inhibitors of TGFβ1, such as those described herein, may be used to treat a disease that is initiated or driven by EMT. Such inhibitors have the ability to suppress expression of myofibroblast/CAF markers in vivo, such as α-SMA, LOXL2, Col1 (Type I collagen), and FN (fibronectin). Thus, TGFβ inhibitors, e.g., TGFβ1 inhibitors, such as Ab6, Ab46, Ab50, a derivative thereof, or an engineered molecule comprising an antigen-binding fragment thereof, may be used for the treatment of a disease characterized by EMT. A therapeutically effective amount of the inhibitor may be an amount sufficient to reduce expression of markers such as α-SMA/ACTA2, LOXL2Col1 (Type I collagen), and FN (fibronectin). In some embodiments, the disease is a proliferative disorder, such as cancer.

Epithelial cells have also been proposed to give rise to myofibroblasts by undergoing the process of EMT in several fibrotic tissues such as kidney, lung and in the liver. EMT takes place when epithelial cells lose their cuboidal shape, lose the expression of adherence and tight junction proteins, which leads to weak cell-cell contacts and reorganization of their actin cytoskeleton; while the cells acquire the expression of mesenchymal proteins (fibronectin, vimentin, N-cadherin), they adopt a fibroblast-like architecture favoring cell migration and invasion. EMT is induced by many growth factors, among them TGFβ being a very potent inducer, which regulate the expression and activity of several transcription factors known as EMT-TFs (Snail1/Snail, Snail2/Slug, ZEB1, ZEB2, Twist1/Twist and more) that are the responsible actors to execute the change in cell differentiation that is EMT. The gene and protein markers used to identify the generation of mesenchymal cells after EMT in the context of fibrosis are FSP1 (Fibroblast-specific protein 1), α-SMA and collagen I along with vimentin and desmin, whose expression increases concomitant with a reduction in levels of expression of epithelial markers (E-cadherin and certain cytokeratins). Cells that co-express epithelial and mesenchymal markers represent an intermediate stage of EMT (reviewed by, for example: Caja et al., Int. J. Mol. Sci. 2018, 19(5), 1294).

Similarly, TGFβ is also a key regulator of the endothelial-to-mesenchymal transition (EndMT) observed in normal development, such as heart formation. However, the same or similar phenomenon is also seen in many disease-associated tissues, such as cancer stroma and fibrotic sites. In some disease processes, endothelial markers such as CD31 become downregulated upon TGFβ1 exposure and instead the expression of mesenchymal markers such as FSP-1, α-SMA/ACTA2 and fibronectin becomes induced. Indeed, stromal CAFs may be derived from vascular endothelial cells. Thus, TGFβ inhibitors, e.g., TGFβ1 inhibitors, such as Ab6, Ab46, Ab50, a derivative thereof, or an engineered molecule comprising an antigen-binding fragment thereof, may be used for the treatment of a disease characterized by EndMT. A therapeutically effective amount of the inhibitor may be an amount sufficient to reduce expression of markers such as FSP-1, α-SMA/ACTA2 and fibronectin. In some embodiments, the disease is a proliferative disorder, such as cancer.

The present disclosure provides a TGFβ inhibitor (e.g., TGFβ1-selective inhibitor such as Ab6) for use in the treatment of a TGFβ-related disorder involving mesenchymal transition (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Diseases Involving Matrix Stiffening and Remodeling

Progression of various TGFβ1-related indications, such as fibrotic conditions and cancer (e.g., tumor growth and metastasis), involves increased levels of matrix components deposited into the ECM and/or maintenance/remodeling of the ECM. It has been reported that increased deposition of ECM components such as collagens can alter the mechanophysical properties of the ECM (e.g., the stiffness of the matrix/substrate) and this phenomenon is associated with TGFβ1 signaling. Applicant previously demonstrated the role of matrix stiffness on integrin-dependent activation of TGFβ, using primary fibroblasts transfected with proTGFβ1 and LTBP1 and grown on silicon-based substrates with defined stiffness (e.g., 5 kPa, 15 kPa or 100 kPa). As disclosed in WO 2018/129329, matrices with greater stiffness enhance TGFβ1 activation, and this can be suppressed by isoform-specific inhibitors of TGFβ1. These observations suggest that TGFβ1 influences ECM properties (such as stiffness), which in turn can further induce TGFβ1 activation, reflective of disease progression.

Thus, TGFβ1 inhibitors, such as Ab6, may be used to block this process to counter disease progression involving ECM alterations, such as fibrosis, tumor growth, invasion, metastasis and desmoplasia. The LTBP-arm of such inhibitors can directly target ECM-associated pro/latent TGFβ1 complexes which are presented by LTBP1 and/or LTBP3, thereby preventing activation/release of the growth factor from the complex in the disease niche. In some embodiments, the TGFβ1 inhibitors may normalize ECM stiffness to treat a disease that involves integrin-dependent signaling. In some embodiments, the integrin comprises an all chain, 31 chain, or both. The architecture of the ECM, e.g., ECM components and organization, can also be altered by matrix-associated proteases. Thus, in some embodiments, the TGFβ1 inhibitors may normalize ECM stiffness to treat a disease that involves protease-dependent signaling associated with disease-associated ECM, e.g., in tumor and fibrotic tissues.

As reviewed in Lampi and Reinhart-King (Science Translational Medicine, 10(422): eaao0475, “Targeting extracellular matrix stiffness to attenuate disease: From molecular mechanisms to clinical trials”), increased stiffness of tissue ECMs occurs during pathological progression of cancer, fibrosis and cardiovascular disease. The mechanical properties associated with the process involve phenotypically converted myofibroblasts, TGFβ and matrix cross-linking. A major cause of increased ECM stiffness during cancer and fibrotic diseases is dysregulated matrix synthesis and remodeling by activated fibroblasts that have de-differentiated into myofibroblasts (e.g., CAFs and FAFs). Remodeling of the tumor stroma and organ fibrosis exhibit striking similarities to the wound healing response, except that in the pathological state the response is sustained. Myofibroblasts are a heterogeneous cell population with pathology-specific precursor cells originating from multiple cell sources, such as bone marrow-derived and tissue resident cells. Commonly used myofibroblast markers include alpha-smooth muscle actin (α-SMA). As shown herein, high-affinity, isoform-specific TGFβ1 inhibitors are able to reduce ACTA2 expression (which encodes α-SMA), collagens, as well as FN (fibronectin) in in vivo studies. Fibronectin is important in the anchoring of LTBP-associated proTGFβ1 complexes onto the matrix structure.

The importance of the TGFβ pathway in ECM regulation is well-established. Because TGFβ1 (and TGFβ3) can be mechanically activated by certain integrins (e.g., av integrins), the integrin-TGFβ1 interaction has become a therapeutic target. For example, a monoclonal antibody to αvβ6 has been investigated for idiopathic lung fibrosis. However, such approach is expected to also interfere with TGFβ3 signaling which shares the same integrin-binding motif, RGD, and furthermore, such antibody will not be effective in blocking TGFβ1 activated via other modes, such as protease-induced activation. In comparison, high-affinity, isoform-specific TGFβ1 inhibitors, such as Ab6, can also block protease-dependent activation of TGFβ1, as well as integrin-dependent activation of TGFβ1. Therefore, such TGFβ1 inhibitors may provide superior attributes. Data presented herein, together with Applicant's previous work, support that high-affinity isoform-selective inhibitors of TGFβ1 may be effective in treating disease associated with ECM stiffening.

Thus, the disclosure includes therapeutic use of isoform-selective inhibitors of TGFβ1 in the treatment of a disease associated with matrix stiffening, or in a method for reducing matrix stiffness, in a subject. Such use comprises administration of a therapeutically effective amount of the isoform-selective inhibitor of TGFβ1, such as Ab6.

The present disclosure provides a TGFβ inhibitor (e.g., TGFβ1-selective inhibitor such as Ab6) for use in the treatment of a TGFβ-related disorder involving matrix stiffening and remodeling (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Diseases Involving Proteases

Activation of TGFβ from its latent complex may be triggered mechanically by integrin in a force-dependent manner, and/or by proteases. Evidence suggests that certain classes of proteases may be involved in the process, including but are not limited to Ser/Thr proteases such as Kallikreins, chemotrypsin, elastases, plasmin, thrombin, as well as zinc metalloproteases of MMP family, such as MMP-2, MMP-9 and MMP-13, and the Adam family of proteases, such as Adam10 and Adam17. MMP-2 degrades the most abundant component of the basement membrane, Collagen IV, raising the possibility that it may play a role in ECM-associated TGFβ1 regulation. MMP-9 has been implicated to play a central role in tumor progression, angiogenesis, stromal remodeling and metastasis, including in carcinoma, such as breast cancer. Thus, protease-dependent activation of TGFβ1 in the ECM may be important for treating ECM-associated diseases such as fibrosis and cancer.

Kallikreins (KLKs) are trypsin- or chymotrypsin-like serine proteases that include plasma Kallikreins and tissue Kallikreins. The ECM plays a role in tissue homeostasis acting as a structural and signaling scaffold and barrier to suppress malignant outgrowth. KLKs may play a role in degrading ECM proteins and other components which may facilitate tumor expansion and invasion. For example, KLK1 is highly upregulated in certain breast cancers and can activate pro-MMP-2 and pro-MMP-9. KLK2 activates latent TGFβ1, rendering prostate cancer adjacent to fibroblasts permissive to cancer growth. KLK3 has been widely studied as a diagnostic marker for prostate cancer (PSA). KLK3 may directly activate TGFβ1 by processing plasminogen into plasmin, which proteolytically cleaves LAP, thereby causing the TGFβ1 growth factor to be released from the latent complex. KLK6 may be a potential marker for Alzheimer's disease.

In some embodiments, an isoform-specific, context-independent inhibitor of TGFβ1 is used for treating a disease associated with Kallikrein or a Kallikrein-like protease. In some embodiments, the TGFβ1 inhibitor is Ab6, or derivatives thereof.

Known activators of TGFβ1, such as plasmin, TSP-1 and αVβ6 integrin, all interact directly with LAP. It is postulated that proteolytic cleavage of LAP may destabilize the LAP-TGFβ interaction, thereby releasing active TGFβ1 (the growth factor domain) from the latent complex. It has been suggested that the region containing the amino acid stretch 54-LSKLRL-59 (SEQ ID NO: 301) is important for maintaining TGFβ1 latency. Thus, agents (e.g., antibodies) that stabilize the interaction, or block the proteolytic cleavage of LAP may prevent TGFβ1 activation.

Many of these proteases associated with pathological conditions (e.g., cancer) function through distinct mechanisms of action. Thus, targeted inhibition of particular proteases, or combinations of proteases, may provide therapeutic benefits for the treatment of conditions involving the protease-TGF1 axis. Accordingly, it is contemplated that inhibitors (e.g., TGFβ1 antibodies) that selectively inhibit protease-induced activation of TGFβ1 may be advantageous in the treatment of such diseases (e.g., cancer). Similarly, selective inhibition of TGFβ1 activation by one protease over another protease may also provide therapeutic benefit, depending on the condition being treated. In preferred embodiments, the isoform-selective activation inhibitor of TGFβ1 is Ab46, Ab50, a derivative thereof, or an engineered molecule comprising an antigen-binding fragment thereof.

Plasmin is a serine protease produced as a precursor form called Plasminogen. Upon release, Plasmin enters circulation and therefore is detected in serum. Elevated levels of serum Plasmin appear to correlate with cancer progression, possibly through mechanisms involving disruption of the extracellular matrix (e.g., basement membrane and stromal barriers) which facilitates tumor cell motility, invasion, and metastasis. Plasmin may also affect adhesion, proliferation, apoptosis, cancer nutrition, oxygen supply, formation of blood vessels, and activation of VEGF (Didiasova et al., Int. J. Mol. Sci, 2014, 15, 21229-21252). In addition, Plasmin may promote the migration of macrophages into the tumor microenvironment (Philips et al., Cancer Res. 2011 Nov. 1; 71(21):6676-83 and Choong et al., Clin. Orthop. Relat. Res. 2003, 415S, S46-S58). Indeed, tumor-associated macrophages (TAMs) are well characterized drivers of tumorigenesis through their ability to promote tumor growth, invasion, metastasis, and angiogenesis.

Plasmin activities have been primarily tied to the disruption of the ECM. However, there is mounting evidence that Plasmin also regulates downstream MMP and TGFβ activation. Specifically, Plasmin has been suggested to cause activation of TGFβ through proteolytic cleavage of the Latency Associated Peptide (LAP), which is derived from the N-terminal region of the TGFβ gene product (Horiguchi et al., J Biochem. 2012 October; 152(4):321-9), resulting in the release of active growth factor. Since TGFβ1 may promote cancer progression, this raises the possibility that plasmin-induced activation of TGFβ may at least in part mediate this process. In preferred embodiments, the isoform-selective activation inhibitor of TGFβ1 is Ab46, Ab50, a derivative thereof, or an engineered molecule comprising an antigen-binding fragment thereof.

TGFβ1 has also been shown to regulate expression of uPA, which is a critical player in the conversion of Plasminogen into Plasmin (Santibanez, Juan F., ISRN Dermatology, 2013: 597927). uPA has independently been shown to promote cancer progression (e.g., adhesion, proliferation, and migration) by binding to its cell surface receptor (uPAR) and promoting conversion of Plasminogen into Plasmin. Moreover, studies have shown that expression of uPA and/or plasminogen activator inhibitor-1 (PAI-1) are predictors of poor prognosis in colorectal cancer (D. Q. Seetoo, et al., Journal of Surgical Oncology, vol. 82, no. 3, pp. 184-193, 2003), breast cancer (N. Harbeck et al., Clinical Breast Cancer, vol. 5, no. 5, pp. 348-352, 2004), and skin cancer (Santibanez, Juan F., ISRN Dermatology, 2013: 597927). Thus, without wishing to be bound by a particular theory, the interplay between Plasmin, TGFβ1, and uPA may create a positive feedback loop towards promoting cancer progression. Accordingly, inhibitors that selectively inhibit Plasmin-dependent TGFβ1 activation may be particularly suitable for the treatment of cancers reliant on the Plasmin/TGFβ1 signaling axis.

Thrombin may be involved in the activation of GARP-associated TGFβ1. Platelets are reported to express GARP-proTGFβ1. Therefore, thrombin may mediate TGFβ1 activation by targeting this axis in an integrin-independent manner.

In one aspect of the disclosure, TGFβ inhibitors such as the isoform-specific inhibitors of TGFβ1 described herein can inhibit protease-dependent activation of TGFβ1. In some embodiments, the inhibitors can inhibit protease-dependent TGFβ1 activation in an integrin-independent manner. In some embodiments, such inhibitors can inhibit TGFβ1 activation irrespective of the mode of activation, e.g., inhibit both integrin-dependent activation and protease-dependent activation of TGFβ1. In some embodiments, the protease is selected from the group consisting of: serine proteases, such as Kallikreins, Chemotrypsin, Trypsin, Elastases, Plasmin, as well as zinc metalloproteases (MMP family) such as MMP-2, MMP-9 and MMP-13.

In some embodiments, the inhibitors can inhibit Plasmin-induced activation of TGFβ1. In some embodiments, the TGFβ inhibitors (e.g., TGFβ1 antibody) can inhibit Plasmin-induced activation of TGFβ1. In some embodiments, the inhibitors can inhibit Plasmin- and integrin-induced TGFβ1 activation. In some embodiments, the inhibitors are monoclonal antibodies that specifically bind TGFβ1. In some embodiments, the antibody is a monoclonal antibody that specifically binds proTGFβ1. In some embodiments, the antibody binds latent proTGFβ1 thereby inhibiting release of mature growth factor from the latent complex. In some embodiments, the high-affinity, context-independent inhibitor of TGFβ1 activation suitable for use in the method of inhibiting Plasmin-dependent activation of TGFβ1 is Ab6 or a derivative or variant thereof. In preferred embodiments, the isoform-selective activation inhibitor of TGFβ1 is Ab46, Ab50, a derivative thereof, or an engineered molecule comprising an antigen-binding fragment thereof.

In some embodiments, the TGFβ inhibitor (e.g., TGFβ1 antibody) inhibits cancer cell migration. In some embodiments, the inhibitor inhibits macrophage migration. In some embodiments, the inhibitor inhibits accumulation of TAMs.

In another aspect, provided herein is a method for treating cancer in a subject in need thereof, the method comprising administering to the subject an effective amount of an TGFβ inhibitor (e.g., TGFβ1 antibody), wherein the inhibitor inhibits protease-induced activation of TGFβ1 (e.g., Plasmin), thereby treating cancer in the subject.

In another aspect, provided herein is a method of reducing tumor growth in a subject in need thereof, the method comprising administering to the subject an effective amount of an TGFβ inhibitor (e.g., TGFβ1 antibody), wherein the inhibitor inhibits protease-induced activation of TGFβ1 (e.g., Plasmin), thereby reducing tumor growth in the subject.

The present disclosure provides a TGFβ inhibitor (e.g., TGFβ1-selective inhibitor such as Ab6) for use in the treatment of a TGFβ-related disorder involving protease(s) (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Treatment Regimen and Administration

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a TGFβ-related indication, such as those noted above. A subject having a TGFβ-related indication can be identified by routine medical examination, e.g., laboratory tests, organ functional tests, CT scans, MRI, or ultrasounds. A subject suspected of having any of such indication might show one or more symptoms of the indication. A subject at risk for the indication can be a subject having one or more of the risk factors for that indication.

As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present invention, the intended purpose may be to inhibit TGFβ-1 activation in vivo, to achieve clinically meaningful outcome associated with the TGFβ-1 inhibition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a TGFβ-related indication. Alternatively, sustained continuous release formulations of an antibody that specifically binds a GARP-TGFβ1 complex, a LTBP1-TGFβ1 complex, a LTBP3-TGFβ1 complex, and/or a LRRC33-TGFβ1 complex may be appropriate. Various formulations and devices for achieving sustained release would be apparent to the skilled artisan and are within the scope of this disclosure.

In one example, dosages for an antibody as described herein may be determined empirically in individuals who have been given one or more administration(s) of the antibody. Individuals are given incremental dosages of the antagonist. To assess efficacy, an indicator of the TGFβ-related indication can be followed. For example, methods for measuring for myofiber damage, myofiber repair, inflammation levels in muscle, and/or fibrosis levels in muscle are well known to one of ordinary skill in the art.

The present disclosure encompasses the recognition that agents capable of modulating the activation step of TGFβs in an isoform-specific manner may provide improved safety profiles when used as a medicament. Accordingly, the disclosure includes antibodies and antigen-binding fragments thereof that specifically bind and inhibit activation of TGFβ1, but not TGFβ2 or TGFβ3, thereby conferring specific inhibition of the TGFβ1 signaling in vivo while minimizing unwanted side effects from affecting TGFβ2 and/or TGFβ3 signaling. In preferred embodiments, the isoform-selective activation inhibitor of TGFβ1 is Ab46, Ab50, a derivative thereof, or an engineered molecule comprising an antigen-binding fragment thereof.

In some embodiments, the antibodies, or antigen binding portions thereof, as described herein, are not toxic when administered to a subject. In some embodiments, the antibodies, or antigen binding portions thereof, as described herein, exhibit reduced toxicity when administered to a subject as compared to an antibody that specifically binds to both TGFβ1 and TGFβ2. In some embodiments, the antibodies, or antigen binding portions thereof, as described herein, exhibit reduced toxicity when administered to a subject as compared to an antibody that specifically binds to both TGFβ1 and TGFβ3. In some embodiments, the antibodies, or antigen binding portions thereof, as described herein, exhibit reduced toxicity when administered to a subject as compared to an antibody that specifically binds to TGFβ1, TGFβ2 and TGFβ3.

Generally, for administration of any of the antibodies described herein, an initial candidate dosage can be about 1-20 mg/kg per administration, with dosing frequency of, e.g., once weekly (QW), once every 2 weeks (Q2W), once every 3 weeks (Q3W), once every 4 weeks (Q4W), monthly, etc. For example, patients may receive an injection of about 1-10 mg/kg per 1 week, per 2 weeks, per 3 weeks, or per 4 weeks, etc., in an amount effective to treat a disease (e.g., fibrosis or cancer) wherein the amount is well-tolerated (within acceptable toxicities or adverse events).

For the purpose of the present disclosure, a typical dosage (per administration, such as an injection and infusion) might range from about 0.1 mg/kg to 30 mg/kg, depending on the factors mentioned above. In some embodiments, a typical dosage (per administration, such as an injection and infusion) might range from about any of 0.1 mg/kg to 1 mg/kg to 2 mg/kg to 3 mg/kg, to 5 mg/kg to 10 mg/kg to 20 mg/kg to 30 mg/kg to 100 mg/kg or more, depending on the factors mentioned above. In some embodiments, suitable dosage include between about 10-3000 mg per dose, e.g., about 10 mg, 30 mg, 40 mg, 50 mg, 60 mg, 70 mg, 80 mg, 90 mg, 100 mg, 120 mg, 150 mg, 180 mg, 200 mg, 240 mg, 300 mg, 400 mg, 500 mg, 800 mg, 1000 mg, 1600 mg, 2000 mg, 2400 mg, etc.). For repeated administrations over several days or longer, depending on the condition, the treatment is sustained until a desired suppression of symptoms occurs or until sufficient therapeutic levels are achieved to alleviate a TGFβ-related indication, or a symptom thereof. For example, suitable effective dosage for Ab6 may be between 1 mg/kg and 30 mg/kg, (e.g., 1-10 mg/kg, 1-15 mg/kg, 3-20 mg/kg, 5-30 mg/kg, etc.) dosed twice a week, once a week, every two weeks, every 4 weeks or once a month. Suitable effective dose for Ab6 includes, about 1 mg/kg, about 3 mg/kg, about 5 mg/kg, about 10 mg/kg, for example, dosed weekly.

In some embodiments, the TGFβ inhibitor is administered at a dose of 500 mg, 600 mg, 700, mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, or 3000 mg. In some embodiments, the TGFβ inhibitor can be administered weekly, biweekly, every three weeks, every four weeks, every five weeks, or every six weeks. For example, SRK-181 (also referred to as Ab6 herein) may be administered at a dose of 1000 mg every 2 weeks or 1500 mg every 3 weeks. In some embodiments, the TGFβinhibitor, e.g., SRK-181, is administered at a dose of 80-3000 mg Q3W or Q2W, more specifically at a dose of 240-2400 mg Q3W or Q2W, e.g., 1500 mg Q3W or 1000 mg Q2W. In some embodiments, the TGFβ inhibitor is administered at the same frequency as a second therapy, e.g., a checkpoint inhibitor therapy, a genotoxic therapy, chemotherapy, and/or radiation therapy. In some embodiments, the TGFβ inhibitor administration starts prior to the initiation of a second therapy. In some embodiments, the TGFβ inhibitor administration starts after the initiation of a second therapy. In some embodiments, the TGFβ inhibitor starts either prior to or after the initiation of the secondary therapy, and continues while the secondary therapy is administered. In some embodiments, the TGFβinhibitor starts either prior to or after the initiation of the secondary therapy, continues while the secondary therapy is administered, and continues even after the second therapy ends.

An exemplary dosing regimen comprises administering an initial dose, followed by one or more of maintenance doses. For example, an initial dose may be between about 1 and 30 mg/kg, for instance, once a week or twice a week. Thereafter, maintenance dose(s) may follow, for example, between about 0.1 and 20 mg/kg, for instance, once a week, every other week, once a month, etc. However, other dosage regimens may be useful, depending on the pattern of pharmacokinetic decay that the practitioner wishes to achieve. Pharmacokinetics experiments have shown that the serum concentration of an antibody disclosed herein (e.g., Ab3) remains stable for at least 7 days after administration to a preclinical animal model (e.g., a mouse model). Without wishing to be bound by any particular theory, this stability post-administration may be advantageous since the antibody may be administered less frequently while maintaining a clinically effective serum concentration in the subject to whom the antibody is administered (e.g., a human subject). In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 1 mg/kg to 30 mg/kg, or from 80 mg to 3000 mg, e.g., 30, 50, 100, 250, 375, 500, 1000, 2000, or 3000 mg, may be administered. The particular dosage regimen, e.g., dose, timing, and repetition, will depend on the particular individual and that individual's medical history, as well as the properties of the individual agents (such as the half-life of the agent, and other relevant considerations).

Serum concentrations of the TGFβ inhibitor antibody that are therapeutically effective to treat a TGFβ1-related indication in accordance with the present disclosure may be at least about 10 μg/mL, e.g., between about 10 μg/mL and 1.0 mg/mL. In some embodiments, effective amounts of the antibody as measured by serum concentrations are about 20-400 μg/mL. In some embodiments, effective amounts of the antibody as measured by serum concentrations are about 100-800 μg/mL. In some embodiments, effective amounts of the antibody as measured by serum concentrations are at least about 20 μg/mL, e.g., at least about 50 μg/mL, 100 μg/mL, 150 μg/mL, or 200 μg/mL. A single dose of Ab6 administered intravenously at 1, 3, 10, 30, or 37.5 mg/kg would result in Cₘₐₓ values of about 25 μg/mL to about 900 μg/mL. Furthermore, in non-human primates, there were no observed toxicities (for example: no cardiotoxicities, hyperplasia and inflammation, dental and gingival findings) associated with Ab6 after maintaining serum concentration levels of about 2,000-3,000 μg/mL for at least 8 weeks. Therefore, about 10-100 fold therapeutic window may be achieved.

According to some embodiments, at least one TGFβ inhibitor, e.g., antibody or antigen-binding portion thereof, described herein, is administered as one or more initial loading dose(s) followed by one or more maintenance or treatment doses. The term “loading dose” refers to a first dose(s) of TGFβ inhibitor, e.g., antibody or antigen-binding portion thereof, which is initially used to treat a subject who may benefit from TGFβ inhibition in vivo. The loading dose may be larger in comparison to the subsequent maintenance or treatment dose(s). The loading dose can be a single dose or, alternatively, a set of doses. For example, a 160 mg loading dose may be administered as a single 160 mg dose, as two doses of 80 mg each, or four doses of 40 mg each. According to some embodiments, a loading dose is subsequently followed by administration of smaller doses of TGFβ inhibitor, e.g., antibody or antigen-binding portion thereof, described herein, e.g., the treatment or maintenance dose(s).

The term “maintenance dose” or “treatment dose” is the amount of TGFβ inhibitor, e.g., antibody or antigen-binding portion thereof, described herein, taken by a subject to maintain or continue a desired therapeutic effect. A maintenance dose can be a single dose or, alternatively, a set of doses. A maintenance dose is administered during the treatment or maintenance phase of therapy. In one embodiment, a maintenance dose(s) is smaller than the induction dose(s) and may be equal to each other when administered in succession.

The term “treatment phase” or “maintenance phase,” as used herein, refers to a period of treatment comprising administration of a TGFβ inhibitor, e.g., antibody or antigen-binding portion thereof, described herein, to a subject in order to maintain a desired therapeutic effect, e.g., improved symptoms associated with a TGFβ-related indication as described herein.

According to some embodiments, the loading dose is administered via intravenous administration, and the maintenance dose is administered by subcutaneous administration. For example, in an exemplary embodiment, a loading dose approach may be applicable in the clinic if the pharmacologically active dose is fairly high and it is desirable to reach a high serum exposure in a short amount of time. Accordingly, an intravenous loading dose could be administered in a medical center, followed by subcutaneous administration of maintenance doses if the antibody half-life is sufficiently long, the antibody is potent enough, and it can be formulated for subcutaneous application.

In some embodiments, an initial loading dose might be first administered, followed by a lower maintenance dose. For example, an initial loading dose might be administered once at a dosage of about 30 mg/kg to about 90 mg/kg intravenously or subcutaneously, followed by a maintenance dosage of about 10 mg/kg to about 30 mg/kg intravenously or subcutaneously.

For the purpose of the present disclosure, the appropriate dosage of an antibody that specifically binds a GARP-TGFβ1 complex, a LTBP1-TGFβ1 complex, a LTBP3-TGFβ1 complex, and/or a LRRC33-TGFβ1 complex will depend on the specific antibody (or compositions thereof) employed, the type and severity of the indication, whether the antibody is administered for preventive or therapeutic purposes, previous therapy, the patient's clinical history and response to the antagonist, and the discretion of the attending physician. In some embodiments, a clinician will administer an antibody that specifically binds a GARP-TGFβ1 complex, a LTBP1-TGFβ1 complex, a LTBP3-TGFβ1 complex, and/or a LRRC33-TGFβ1 complex, until a dosage is reached that achieves the desired result. Administration of an antibody that specifically binds a GARP-TGFβ1 complex, a LTBP1-TGFβ1 complex, a LTBP3-TGFβ1 complex, and/or a LRRC33-TGFβ1 complex can be continuous or intermittent, depending, for example, upon the recipient's physiological condition, whether the purpose of the administration is therapeutic or prophylactic, and other factors known to skilled practitioners. The administration of antibody that specifically binds a GARP-TGFβ1 complex, a LTBP1-TGFβ1 complex, a LTBP3-TGFβ1 complex, and/or a LRRC33-TGFβ1 complex may be essentially continuous over a preselected period of time or may be in a series of spaced dose, e.g., either before, during, or after developing a TGFβ-related indication.

In order to minimize potential risk and adverse events associated with TGFβ inhibition, a TGFβ inhibitor such as any one of the antibodies disclosed herein may be administered intermittently. For instance, the TGFβinhibitor may be administered on an “as needed” basis in a therapeutically effective amount sufficient to achieve and maintain clinical benefit (e.g., reduction of tumor volume and/or reversal or reduction of immunosuppression). In some embodiments, administration of a TGFβ inhibitor such as any one of the antibodies disclosed herein (e.g., a TGFβ1 inhibitor, e.g., Ab6) may be used in combination with a method of determining or monitoring therapeutic efficacy (e.g., measuring of circulating MDSCs). In some embodiments, the TGFβ inhibitor is administered in patients only when clinical benefit from additional doses of the TGFβ inhibitor is determined, e.g., when an elevation in circulating MDSCs is detected.

In some instances, intermittent dosing regimen is considered. Intermittent dosing regimen may be particularly appropriate where patients receive a therapy comprising a TGFβ inhibitor that is not selective for TGFβ1, because there may be a greater risk of toxicity. To mitigate or manage such risk, the non-selective TGFβinhibitor may be administered infrequently or intermittently, for example on an “as-needed” basis.

Based on the observation that inhibiting TGFβ3 can increase collagen deposition or accumulation in fibrosis, add-on therapy comprising a TGFβ1-selective inhibitor (such as the novel antibodies disclosed herein) may be considered for patients who are treated with a TGFβ inhibitor with TGFβ3-inhibiting activity, e.g., inhibitors of TGFβ1/2/3, TGFβ1/3 and TGFβ3. Examples of TGFβ inhibitors with TGFβ3-inhibiting activity include but are not limited to: low molecular weight antagonists of TGFβ receptors, e.g., ALK5 antagonists, such as Galunisertib (LY2157299 monohydrate); monoclonal antibodies (such as neutralizing antibodies) that inhibit all three isoforms (“pan-inhibitor” antibodies) (see, for example, WO 2018/134681); monoclonal antibodies that preferentially inhibit two of the three isoforms (e.g., TGFβ1/3 (for example WO 2006/116002); and engineered molecules (e.g., fusion proteins) such as ligand traps (for example, WO 2018/029367; WO 2018/129331 and WO 2018/158727). In some embodiments, the ligand trap comprises the structure in accordance with the disclosure of WO/2018/15872. In some embodiments, the ligand trap comprises the structure in accordance with the disclosure of WO 2018/029367; WO 2018/129331. In some embodiments, the ligand trap is a construct known as M7824. In some embodiments, the ligand trap is a construct known as AVID200. In some embodiments, the neutralizing pan-TGFβ antibody is GC1008 or a derivative thereof. In some embodiments, such antibody comprises the sequence in accordance with the disclosure of WO/2018/134681.

In some embodiments, the antibody is a neutralizing antibody that specifically binds both TGFβ1 and TGFβ3. In some embodiments such antibody preferentially binds TGFβ1 over TGFβ3. For example, the antibody comprises the sequence in accordance with the disclosure of WO/2006/116002. In some embodiments, the antibody is 21D1.

The add-on therapy is aimed to counter or overcome the pro-fibrotic effect of TGFβ3 inhibition. In some embodiments, the patient has a fibrotic disorder or is at risk of developing a fibrotic disorder and/or a cardiovascular disease. Accordingly, the disclosure includes a TGFβ1-selective inhibitor for use in an add-on therapy of a subject treated with a TGFβ3 inhibitor, in an amount sufficient to reduce pro-fibrotic effects of the TGFβ3 inhibitor. In some embodiments, the subject has fibrosis, e.g., lung fibrosis. In some embodiments, the TGFβ1-selective inhibitor is Ab2, Ab46, Ab50, or a derivative thereof. In preferred embodiments, the TGFβ1-selective inhibitor is Ab46.

Without being bound by theory, in some embodiments, sparing of TGFβ inhibitors with anti-TGFβ3 activities may be especially useful for treating patients having or are at risk of developing a fibrotic condition and/or a cardiovascular disease. Accordingly, the disclosure herein includes a TGFβ inhibitor for use wherein the patient has or is at risk of developing a fibrotic condition and/or a cardiovascular disease, wherein the fibrotic condition is pulmonary fibrosis

As used herein, the term “treating” refers to the application or administration of a composition including one or more active agents to a subject, who has a TGFβ-related indication, a symptom of the indication, or a predisposition toward the indication, with the purpose to cure, heal, alleviate, relieve, alter, remedy, ameliorate, improve, or affect the indication, the symptom of the indication, or the predisposition toward the indication.

Alleviating a TGFβ-related indication with an antibody that specifically binds a GARP-TGFβ1 complex, a LTBP1-TGFβ1 complex, a LTBP3-TGFβ1 complex, and/or a LRRC33-TGFβ1 complex includes delaying the development or progression of the indication, or reducing indication's severity. Alleviating the indication does not necessarily require curative results. As used therein, “delaying” the development of an indication associated with a TGFβ-related indication means to defer, hinder, slow, retard, stabilize, and/or postpone progression of the indication. This delay can be of varying lengths of time, depending on the history of the indication and/or individuals being treated. A method that “delays” or alleviates the development of an indication, or delays the onset of the indication, is a method that reduces probability of developing one or more symptoms of the indication in a given time frame and/or reduces extent of the symptoms in a given time frame, when compared to not using the method. Such comparisons are typically based on clinical studies, using a number of subjects sufficient to give a statistically significant result.

### Diagnostics, Patient Selection, Monitoring

Therapeutic methods that include TGFβ inhibition therapy may comprise diagnosis of a TGFβ indication and/or selection of patients likely to respond to such therapy. Additionally, patients who receive the TGFβ inhibitor may be monitored for therapeutic effects of the treatment, which typically involves measuring one or more suitable parameters which are indicative of the condition and which can be measured (e.g., assayed) before and after the treatment and evaluating treatment-related changes in the parameters. For example, such parameters may include levels of biomarkers present in biological samples collected from the patients. Biomarkers may be RNA-based, protein-based, cell-based and/or tissue-based. For example, genes that are overexpressed in certain disease conditions may serve as the biomarkers to diagnose and/or monitor the disease or response to the therapy. Cell-surface proteins of disease-associated cell populations may serve as biomarkers. Such methods may include the direct measurements of disease parameters indicative of the extent of the particular disease, such as tumor size/volume and extent of fibrosis. Any suitable sampling methods may be employed, such as serum/blood samples, biopsies, and imaging. The biopsy may include tissue biopsies (such as tumor biopsy, e.g., core needle biopsy) and liquid biopsies. In some embodiments, the liquid biopsies involve measuring or detecting immune cells in blood (e.g., in a serum, and/or plasma sample), e.g., circulating lymphocytes, such as circulating MDSCs. In some embodiments, circulating MDSC levels serve as a predictive biomarker.

While biopsies have traditionally been the standard for diagnosing and monitoring various diseases, such as fibrosis (e.g., organ fibrosis) and proliferative disorders (e.g., cancer), less invasive alternatives may be preferred. For example, many non-invasive in vivo imaging techniques may be used to diagnose, monitor, and select patients for treatment. Thus, the disclosure includes the use of in vivo imaging techniques to diagnose and/or monitor disease in a patient or subject. In some embodiments, the patient or subject is receiving an isoform-specific TGFβ1 inhibitor as described herein. In other embodiments, an in vivo imaging technique may be used to select patients for treatment with an isoform-specific TGFβ1 inhibitor. In some embodiments, such techniques may be used to determine if or how patients respond to a therapy, e.g., TGFβ1 inhibition therapy.

Exemplary in vivo imaging techniques used for the methods include, but are not limited to X-ray radiography, magnetic resonance imaging (MRI), medical ultrasonography or ultrasound, endoscopy, elastography, tactile imaging, thermography, medical photography. Other imaging techniques include nuclear medicine functional imaging, e.g., positron emission tomography (PET) and Single-photon emission computed tomography (SPECT). Methods for conducting these techniques and analyzing the results are known in the art.

Non-invasive imaging techniques commonly used to diagnose and monitor cancer include, but are not limited to: magnetic resonance imaging (MRI), computed tomography (CT), ultrasound, positron emission tomography (PET), single-photon emission computed tomography (SPECT), fluorescence reflectance imaging (FRI), and fluorescence mediated tomography (FMT). Hybrid imaging platforms may also be used to diagnose and monitor cancer. For example, hybrid techniques include, but are not limited to: PET-CT, FMT-CT, FMT-MRI, and PET-MRI. Dynamic contrast enhanced MRI (DCE-MRI) is another imaging technique commonly used to detect breast cancers. Methods for conducting these techniques and analyzing the results are known in the art.

Non-invasive imaging techniques commonly used to diagnosis and monitor fibrosis include, but are not limited to: ultrasound (e.g., conventional or contrast-enhanced ultrasound), ultrasound elastography (e.g., transient elastography, point shear wave elastography and 2D-shear wave elastography), CT scan (e.g., conventional CT or CT perfusion imaging), magnetic resonance imaging (MRI) (e.g., conventional MRI, Magnetic resonance elastography, diffusion weighted magnetic resonance imaging, gadoxetic acid disodium, and magnetic resonance perfusion imaging).

In some embodiments, non-invasive imaging techniques are used to assess levels of liver fibrosis or hepatic steatosis. For example, imaging techniques particularly useful to assess liver fibrosis may include but are not limited to: FibroScan (transient elastography; TE), point shear wave elastography (pSWE; a.k.a. acoustic radiation force impulse (ARFI)), 2D-3D SWE, magnetic resonance elastography (MRE), and multiparameteric MRI. Imaging techniques particularly useful to assess hepatic steatosis may include but are not limited to: ultrasonography, controlled attenuation parameter (CAP) elastography, MRI-estimated proton density fat fraction (MRI-PDFF), and magnetic resonance spectroscopy (MRS). In some embodiments, the in vivo imaging technique is used to assess liver stiffness. In some embodiments, the in vivo imaging technique is used to detect and assess intrahepatic triglyceride levels. In some embodiments, in vivo imaging technique is used to assess liver surface nodularity (LSN; a.k.a. “liver score”), liver stiffness, and/or liver segmental volume ratio (LSVR), which are all beneficial in the staging of hepatic fibrosis and sub-staging cirrhosis. Methods for conducting these techniques and analyzing the results are known in the art. More recently, non-invasive imaging methods are being developed which will allow the detection of cells of interest (e.g., cytotoxic T cells, macrophages, and cancer cells) in vivo. See, for example, www.imaginab.com/technology/; Tavare et al., (2014) PNAS, 111(3): 1108-1113; Tavare et al., (2015) J Nucl Med 56(8): 1258-1264; Rashidian et al., (2017) J Exp Med 214(8): 2243-2255; Beckford Vera et al., (2018) PLoS ONE 13(3): e0193832; and Tavare et al., (2015) Cancer Res 76(1): 73-82, each of which is incorporated herein by reference. So-called “T-cell tracking” is aimed to detect and localize anti-tumor effector T-cells in vivo. This may provide useful insights into understanding the immunosuppressive phenotype of solid tumors. Tumors that are well-infiltrated with cytotoxic T cells (“inflamed” or “hot” tumors) are likely to respond to cancer therapies such as checkpoint blockade therapy (CBT). On the other hand, tumors with immunosuppressive phenotypes tend to have poor T-cell infiltration even when there is an anti-tumor immune response. These so-called “immune excluded” tumors likely fail to respond to cancer therapies such as CBT. T-cell tracking techniques may reveal these different phenotypes and provide information to guide in therapeutic approach that would likely benefit the patients. For example, patients with an “immune excluded” tumor are likely to benefit from a TGFβ inhibitor therapy such as a TGFβ1 inhibitor therapy to help reverse the immunosuppressive phenotype. It is contemplated that similar techniques may be used to diagnose and monitor other diseases, for example, fibrosis. Typically, antibodies or antibody-like molecules engineered with a detection moiety (e.g., radiolabel, fluorescence, etc.) can be infused into a patient, which then will distribute and localize to sites of the particular marker (for instance CD8+ and M2 macrophages).

Non-invasive in vivo imaging techniques may be applied in a variety of suitable methods for purposes of diagnosing patients; selecting or identifying patients who are likely to benefit from TGFβ inhibitor therapy such as TGFβ1 inhibitor therapy; and/or, monitoring patients for therapeutic response upon treatment. Any cells with a known cell-surface marker may be detected/localized by virtue of employing an antibody or similar molecules that specifically bind to the cell marker. Typically, cells to be detected by the use of such techniques are immune cells, such as cytotoxic T lymphocytes, regulatory T cells, MDSCs, disease-associated macrophages (M2 macropahges such as TAMs and FAMs), NK cells, dendritic cells, and neutrophils.

Non-limiting examples of suitable immune cell markers include monocyte markers, macrophage markers (e.g., M1 and/or M2 macrophage markers), CTL markers, suppressive immune cell markers, MDSC markers (e.g., markers for G- and/or M-MDSCs), including but are not limited to: CD8, CD3, CD4, CD11 b, CD33, CD163, CD206, CD68, CD14, CD15, CD66b, CD34, CD25, and CD47. In some embodiments, LRRC33 is used as a marker for MDSCs. In some embodiments, LRRC33 is used as a marker for MDSCs in circulation. In some embodiments, a reduction in circulating MDSC levels following treatment with a TGFβ inhibitor (e.g., a TGFβ1-selective inhibitor, e.g., Ab6) may be indicative of therapeutic efficacy. In some embodiments, levels of circulating MDSCs may be used to predict, determine, and monitor pharmacological effects of treatment comprising a dose of TGFβ inhibitor (e.g., a TGFβ1-selective inhibitor, e.g., Ab6). Thus, LRRC33 levels in blood samples may serve as a predictive biomarker for assessing therapeutic response in conditions associated with immune suppression.

In vivo imaging techniques described above may be employed to detect, localize and/or track certain MDSCs in a patient diagnosed with a TGFβ1-associated disease, such as cancer and fibrosis. Healthy individuals have no or low frequency of MDSCs in circulation. With the onset of or progression of such a disease, elevated levels of circulating and/or disease-associated MDSCs may be detected. For example, CCR2-positive M-MDSCs have been reported to accumulate to tissues with inflammation and may cause progression of fibrosis in the tissue (such as pulmonary fibrosis), and this is shown to correlate with TGFβ1 expression. Similarly, MDSCs are enriched in a number of solid tumors (including triple-negative breast cancer) and in part contribute to the immunosuppressive phenotype of the TME. Therefore, treatment response to TGFβ inhibition therapy such as a TGFβ1 inhibitor therapy according to the present disclosure may be monitored by localizing, tracking, or measuring MDSCs. Reduction of or low frequency of detectable MDSCs is typically indicative of therapeutic benefits or better prognosis.

Accordingly, the disclosure also includes a method for treating a TGFβ1-related disease or condition which may comprise the following steps: i) selecting a patient diagnosed with a TGFβ1-related disease or condition; and, ii) administering to the patient an antibody or the fragment encompassed herein in an amount effective to treat the disease or condition. In some embodiments, the selection step (i) comprises detection of disease markers (e.g., fibrosis or cancer markers as described herein), wherein optionally the detection comprises a biopsy analysis, serum marker analysis, and/or in vivo imaging. In some embodiments, the selection step (i) comprises an in vivo imaging technique as described herein.

In some embodiments, the TGFβ1-related disease or condition is a fibrotic condition. In some embodiments, the selection step (i) comprises detection of myofibroblasts cells, or one or more markers thereof. In some embodiments, the detection comprises a biopsy analysis, serum marker analysis, and/or in vivo imaging. In some embodiments, the in vivo imaging comprises ultrasound, ultrasound elastography, CT scan, MRI, PET-SPECT, optical fluorescence/bioluminescence FibroScan (TE), pSWE, 2D-3D SWE, MRE, ultrasonography, CAP, MRI-PDFF, and/or MRS. In some embodiments, in vivo imaging comprises direct or indirect labeling of immune cells or antibody that binds a cell-surface marker of immune cells. In some embodiments, the in vivo imaging comprises the use of a tracer.

The disclosure also includes a method for treating cancer which may comprise the following steps: i) selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. Preferably, the patient has received, or is a candidate for receiving a cancer therapy such as immune checkpoint inhibition therapies (e.g., PD-(L)1 antibodies), chemotherapies, radiation therapies, engineered immune cell therapies, and cancer vaccine therapies. In some embodiments, the selection step (i) comprises detection of immune cells or one or more markers thereof, wherein optionally the detection comprises a tumor biopsy analysis, serum marker analysis, and/or in vivo imaging. In some embodiments, the selection step (i) comprises an in vivo imaging technique as described here. In some embodiments, the method further comprises monitoring for a therapeutic response as described herein. In certain embodiments, circulating MDSCs, such as G-MDSCs and M-MDSCs, are measured before and after (e.g., 1-7 days or 1-10 weeks before or after) administering a therapeutically effective dose of a TGFβ inhibitor such as a TGFβ1 inhibitor described herein as an indicator of therapeutic efficacy and/or a predictor of response.

In some embodiments, in vivo imaging is performed for monitoring a therapeutic response to the TGFβ1 inhibition therapy in the subject. The in vivo imaging can comprise any one of the imaging techniques described herein and measure any one of the markers and/or parameters described herein. In the case of cancer, the therapeutic response may comprise conversion of an immune excluded tumor into an inflamed tumor (which correlates with increased immune cell infiltration into a tumor), reduced tumor size, and/or reduced disease progression. Increased immune cell infiltration may be visualized by increased intratumoral immune cell frequency or degree of detection signals, such as radiolabeling and fluorescence.

In some embodiments, the in vivo imaging used for diagnosing, selecting, treating, or monitoring patients, comprises MDSC tracking, such as G-MDSCs (also known as PMN-MDSCs) and M-MDSCs. For example, MDSCs may be enriched at a disease site (such as fibrotic tissues and solid tumors) at the baseline. Upon therapy (e.g., TGFβ1 inhibitor therapy), fewer MDSCs may be observed, as measured by reduced intensity of the label (such as radioisotope and fluorescence), indicative of therapeutic effects. In some embodiments, circulating MDSCs, including circulating G-MDSCs and M-MDSCs, may be detected in the blood or a blood component of the subject receiving a TGFβ inhibitor, e.g., Ab6. In some embodiments, MDSCs are detected, measured, or quantified by the use of an antibody that binds LRRC33.

In certain embodiments, assays useful in determining the efficacy and/or therapeutic response in a subject treated with a TGFβ inhibitor (e.g., Ab6) include, but are not limited to, immunohistochemical or immunofluorescence analyses of certain immune cell markers (e.g., flow cytometry) known in the art for measuring levels of circulating MDSCs (e.g., G-MDSCs and M-MDSCs). In some embodiments, human G-MDSCs may be identified by the expression of the surface markers CD11b, CD33, CD15, and CD66b. In some embodiments, human G-MDSCs may also express HLA-DR, LOX-1, and/or Arginase. In some embodiments, M-MDSCs may be identified by the expression of surface markers CD11b, CD33 and CD14. In some embodiments, M-MDSCs may also express HLA-DR. In some embodiments, the TGFβ inhibitors such as those encompassed herein may be used to detect reduction in circulatory MDSCs, but not levels of other circulatory monocytes, after administration to a patient in need thereof. In some embodiments, circulatory MDSCs are measured by the expression of cell surface expressed LRRC33.

In some embodiments, the in vivo imaging comprises tracking or localization of LRRC33-positive cells. LRRC33-positive cells include, for example, MDSCs and activated M2-like macrophages (e.g., TAMs and activated macrophages associated with fibrotic tissues). For example, LRRC33-positive cells may be enriched at a disease site (such as fibrotic tissues and solid tumors) at the baseline. Upon therapy (e.g., TGFβ1 inhibitor therapy), fewer cells expressing cell surface LRRC33 may be observed, as measured by reduced intensity of the label (such as radioisotope and fluorescence), indicative of therapeutic effects.

In some embodiments, the in vivo imaging techniques described herein may comprise the use of PET-SPECT, MRI and/or optical fluorescence/bioluminescence in order to detect cells of interest.

In some embodiments, labeling of antibodies or antibody-like molecules with a detection moiety may comprise direct labeling or indirect labeling.

In some embodiments, the detection moiety may be a tracer. In some embodiments, the tracer may be a radioisotope, wherein optionally the radioisotope may be a positron-emitting isotope. In some embodiments, the radioisotope is selected from the group consisting of: 18F, 11C, 13N, 15O, 68Ga, 177Lu, 18F and 89Zr.

Thus, such methods may be employed to carry out in vivo imaging with the use of labeled antibodies in immune-PET.

Accordingly, the disclosure also includes a method for treating a TGFβ1 indication in a subject, which method comprises a step of diagnosis, patient selection, and/or monitoring therapeutic effects using an imaging technique. In some embodiments, a TGFβ inhibitor such as an isoform-selective TGFβ1 inhibitor according to the present disclosure is used in the treatment of a TGFβ1 indication, wherein the treatment comprises administration of an effective amount of the TGFβ inhibitor (e.g., Ab6) to treat the indication, and further comprising a step of monitoring therapeutic effects in the subject by in vivo imaging. Optionally, the subject may be selected as a candidate for receiving the TGFβ inhibitor therapy (e.g., a TGFβ1 inhibitor therapy), using a diagnostic or selection step that comprises in vivo imaging. The TGFβ1 indication may be a proliferative disorder (such as cancer with a solid tumor and myelofibrosis). In some embodiments, the TGFβ1 indication is a fibrotic disorder (such as organ fibrosis).

In some embodiments, the subject has cancer, wherein the method comprises the following steps: i) selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. Preferably, the patient has received, or is a candidate for receiving a cancer therapy such as immune checkpoint inhibition therapies (e.g., PD-(L)1 antibodies), chemotherapies, radiation therapies, engineered immune cell therapies, and cancer vaccine therapies. In some embodiments, the selection step (i) comprises detection of immune cells or one or more markers thereof, wherein optionally the detection comprises a tumor biopsy analysis, serum marker analysis, and/or in vivo imaging. In some embodiments, the selection step (i) comprises an in vivo imaging technique as described here. In some embodiments, the method further comprises monitoring for a therapeutic response as described herein. In some embodiments, the TGFβ1 indication is a fibrotic disorder (such as organ fibrosis).

### Inhibition of TGFβ1 Activity

Methods of the present disclosure include methods of inhibiting TGFβ1 growth factor activity in one or more biological system. Such methods may include contacting one or more biological system with an antibody and/or composition of the disclosure. In some cases, these methods include modifying the level of free growth factor in a biological system (e.g., in a cell niche or subject). Antibodies and/or compositions according to such methods may include, but are not limited to biomolecules, including, but not limited to recombinant proteins, protein complexes and/or antibodies, or antigen portions thereof, described herein.

In some embodiments, methods of the present disclosure may be used to reduce or eliminate growth factor activity, termed “inhibiting methods” herein. Some such methods may comprise mature growth factor retention in a TGFβ complex (e.g., a TGFβ1 complexed with GARP, LTBP1, LTBP3 and/or LRRC33) and/or promotion of reassociation of growth factor into a TGFβ complex. In some cases, inhibiting methods may comprise the use of an antibody disclosed herein. According to some inhibiting methods, one or more inhibiting antibody is provided.

In some embodiments, antibodies, antigen-binding portions thereof, and compositions of the disclosure may be used for inhibiting TGFβ1 activation. In some embodiments, provided herein is a method for inhibiting TGFβ1 activation comprising exposing a GARP-TGFβ1 complex, a LTBP1-TGFβ1 complex, a LTBP3-TGFβ1 complex, and/or a LRRC33-TGFβ1 complex to an antibody, an antigen-binding portion thereof, or a pharmaceutical composition described herein. In some embodiments, the antibody, antigen-binding portion thereof, or pharmaceutical composition, inhibits the release of mature TGFβ1 from the GARP-TGFβ1 complex, the LTBP1-TGFβ1 complex, a LTBP3-TGFβ1 complex, and/or the LRRC33-TGFβ1 complex. In some embodiments, the method is performed in vitro. In some embodiments, the method is performed in vivo. In some embodiments, the method is performed ex vivo.

In some embodiments, the GARP-TGFβ1 complex or the LRRC33-TGFβ1 complex is present at the outer surface of a cell.

In some embodiments, the cell expressing the GARP-TGFβ1 complex or the LRRC33-TGFβ1 complex is a fibroblast, a myofibroblast, a macrophage, a T-cell, a monocyte, a dendritic cell, an antigen presenting cell, a neutrophil, a myeloid-derived suppressor cell (MDSC), a lymphocyte, a mast cell, or a microglial cell. The myofibroblast may be a fibrosis-associated fibroblast (FAF) of a cancer-associated fibroblasts (CAF). The T-cell may be a regulatory T cell (e.g., immunosuppressive T cell). The neutrophil may be an activated neutrophil. The macrophage may be a resident macrophage (e.g., a liver kupffer cell) or an infiltrating macrophage. The macrophage may be an activated (e.g., polarized) macrophage, including profibrotic and/or tumor-associated macrophages (TAM), e.g., M2c subtype and M2d subtype macrophages. In some embodiments, macrophages are exposed to tumor-derived factors (e.g., cytokines, growth factors, etc.) which may further induce pro-cancer phenotypes in macrophages. In some embodiments, such tumor-derived factor is CSF-1/M-CSF.

In some embodiments, the GARP-TGFβ1 complex, the LTBP1-TGFβ1 complex, the LTBP3-TGFβ1 complex, and/or the LRRC33-TGFβ1 complex is bound to an extracellular matrix.

In some embodiments, provided herein is a method for reducing TGFβ1 protein activation in a subject comprising administering an antibody, an antigen-binding portion thereof, or a pharmaceutical composition described herein to the subject, thereby reducing TGFβ1 protein activation in the subject. In some embodiments, the subject has or is at risk of having fibrosis. In some embodiments, the subject has or is at risk of having cancer. In some embodiments, the subject has or is at risk of having dementia.

In some embodiments, the antibodies, or the antigen-binding portions thereof, as described herein, reduce the suppressive activity of regulatory T cells (Tregs).

Cell-Based Assays for Measuring TGFβ Activation and/or Inhibitory Potency

Activation of TGFβ (and inhibition thereof by a TGFβ test inhibitor, such as an antibody) may be measured by any suitable method known in the art. For example, integrin-mediated activation of TGFβ can be utilized in a cell-based assay, such as the “CAGA12” luciferase assay, described in more detail herein. As shown, such an assay system may comprise the following components: i) a source of TGFβ (recombinant, endogenous or transfected); ii) a source of activator such as integrin (recombinant, endogenous, or transfected); and iii) a reporter system that responds to TGFβ activation, such as cells expressing TGFβ receptors capable of responding to TGFβ and translating the signal into a readable output (e.g., luciferase activity in CAGA12 cells or other reporter cell lines). In some embodiments, the reporter cell line comprises a reporter gene (e.g., a luciferase gene) under the control of a TGFβ-responsive promoter (e.g., a PAI-1 promoter). In some embodiments, certain promoter elements that confer sensitivity may be incorporated into the reporter system. In some embodiments, such promoter element is the CAGA12 element. Reporter cell lines that may be used in the assay have been described, for example, in Abe et al., (1994) Anal Biochem. 216(2): 276-84, incorporated herein by reference. In some embodiments, each of the aforementioned assay components are provided from the same source (e.g., the same cell). In some embodiments, two of the aforementioned assay components are provided from the same source, and a third assay component is provided from a different source. In some embodiments, all three assay components are provided from different sources. For example, in some embodiments, the integrin and the latent TGFβ complex (proTGFβ and a presenting molecule) are provided for the assay from the same source (e.g., the same transfected cell line). In some embodiments, the integrin and the TGF are provided for the assay from separate sources (e.g., two different cell lines, a combination of purified integrin and a transfected cell). When cells are used as the source of one or more of the assay components, such components of the assay may be endogenous to the cell, stably expressed in the cell, transiently transfected, or any combination thereof. TGFβ activation may be measured in a cell-based assay. Inhibitory potency of TGFβ inhibitors may be evaluated using cell-based potency assays, such as so-called CAGA reporter assays, as previously described. See, for example, the CAGA-reporter assays disclosed in WO 2018/129329 and WO 2020/014460.

Generally, such reporter assay comprises at least three assay components: a source of proTGFb1; activation means (e.g., integrins that bind the RGD motif); and readout means (e.g., TGFβ-sensitive reporter genes).

In some embodiments, such cell-based assay comprises a single-cell reporter assay, in which the three assay components are expressed by a single clone, preferably as a stably transfected cell line.

In some embodiments, the cell line is an LN229 cell line, which is a human glioblastoma cell line. In some embodiments, the cell line is stably transfected with proTGFβ1, alpha-v/beta-8 integrin, TGFβ receptors, and CAGA12 promoter operatively linked to a reporter gene, preferably the luciferase gene. In preferred embodiments, proTGFβ1 expression is driven by an inducible promoter, wherein optionally the inducible promoter is regulated by tetracycline. In preferred embodiments, the inducible promoter is doxycycline-inducible.

The development of novel context-dependent cell-based assays of TGFβ1 activation is described in U.S. Provisional Patent Application No. 62/538,476 and the corresponding International Application Pub. No. WO 2019/023661, each of which are incorporated by reference in their entirety herein. Previous assay formats could not differentiate between the activation of proTGFβ1 presented by endogenous presenting molecules and the activation of proTGFβ1 presented by exogenous LTBPs. By directly transfecting integrin-expressing cells, the novel assays disclosed in International Application Pub. No. WO 2019/023661, and used herein, establish a window between endogenous presenter-proTGFβ1 activity and exogenous LTBP-proTGFβ1 activity.

As opposed to GARP- or LRRC33-proTGFβ1 complexes, which are presented on the surface of cells, LTBP-proTGFβ1 complexes are embedded in the extracellular matrix. Thus, the assay plate coating is an important component of the assay when assessing activation of proTGFβ1 in complex with LTBP (e.g., LTBP1/3). In this regard, it has been shown, fibronectin and fibronectin are two ECM components that appear to be critical for LTBP association with the matrix and activation of latent TGFβ (Robertson et al., Matrix Biol. 2015 September; 47:44-53). For example, LTBP3 ECM-incorporation appears to be dependent on fibrillin expression in both in vitro and in vivo models (Zilberberg et al., J Cell Physiol. 2012; 227(12):3828-3836).

On the other hand, LTBP1 has been shown to interact with fibrillin microfibrils and fibronectin via its C- and N-termini, respectively (Dallas et al., J Biol Chem. 2005; 280(19):18871-18880; Fontana et al., FASEB J. 2005; 19(13):1798-1808; and Kantola et al., Exp Cell Res. 2008; 314(13):2488-2500). Moreover, in the absence of fibrillin, LTBP1 still co-localizes with fibronectin fibers (Robertson et al., Matrix Biol. 2015 September; 47:44-53). LTBP1 has also been shown to interact with ADAMTSL2 and 3 (Sengle et al., PloS Genet. 2012; 8(1):e1002425), IGFBP3 (Gui and Murphy, Mol Cell Biochem. 2003; 250(1-2):189-195), fibulin-4 (Massam-Wu et al., J Cell Sci. 2010 Sep. 1; 123(Pt 17):3006-18), and heparin (Chen et al., J Biol Chem. 2007; 282(36):26418-26430).

Beyond traditional ECM components/proteins, tissue transglutaminase (TG2) may also play a critical role in TGFβ localization in the ECM. TG2 is known to catalyze inter- and intramolecular isopeptide bonds which cross-link ECM fibrils, effectively stiffening the ECM and protecting the ECM from proteolytic degradation (Benn et al., Current Opinion in Biomedical Engineering., 2019, https://doi.org/10.1016/j.cobme.2019.06.003). Moreover, TG2 can cross-link LTBP1 to the ECM, thus promoting a matrix reservoir of TGFβ (Nunes et al., J Cell Biol. 1997; 136(5):1151-1163).

Accordingly, the N-terminus of LTBPs may be covalently bound to the ECM via an isopeptide bond, the formation of which may be catalyzed by transglutaminases. The structural integrity of the ECM is believed to be important in mediating LTBP-associated TGFβ1 activity. For example, stiffness of the matrix can significantly affect TGFβ1 activation. In addition, incorporating fibronectin and/or fibrillin in the scaffold may significantly increase the LTBP-mediated TGFβ1 activation. Similarly, presence of fibronectin and/or fibrillin in LTBP assays (e.g., cell-based potency assays) may increase an assay window.

Accordingly, a cell-based assay for measuring TGFβ1 activation may comprise the following components: i) a source of TGFβ (recombinant, endogenous or transfected); ii) a source of activator such as integrin (recombinant, endogenous, or transfected); and iii) a reporter system that responds to TGFβ activation, such as cells expressing TGFβ receptors capable of responding to TGFβ and translating the signal into a readable output (e.g., luciferase activity in CAGA12 cells or other reporter cell lines). In some embodiments, the reporter cell line comprises a reporter gene (e.g., a luciferase gene) under the control of a TGFβ-responsive promoter (e.g., a PAI-1 promoter). In some embodiments, certain promoter elements that confer sensitivity may be incorporated into the reporter system. In some embodiments, such promoter element is the CAGA12 element. Reporter cell lines that may be used in the assay have been described, for example, in Abe et al., (1994) Anal Biochem. 216(2): 276-84, incorporated herein by reference. In some embodiments, each of the aforementioned assay components are provided from the same source (e.g., the same cell). In some embodiments, two of the aforementioned assay components are provided from the same source, and a third assay component is provided from a different source. In some embodiments, all three assay components are provided from different sources. For example, in some embodiments, the integrin and the latent TGFβ complex (proTGFβ and a presenting molecule) are provided for the assay from the same source (e.g., the same transfected cell line). In some embodiments, the integrin and the TGF are provided for the assay from separate sources (e.g., two different cell lines, a combination of purified integrin and a transfected cell). When cells are used as the source of one or more of the assay components, such components of the assay may be endogenous to the cell, stably expressed in the cell, transiently transfected, or any combination thereof. In some embodiments, the assay is performed in a tissue culture plate or dish. In some embodiments, the tissue culture plate or dish is coated with a component of the extracellular matrix (ECM). In some embodiments, the tissue culture plate or dish is coated with fibronectin and/or fibrillin. In some embodiments, the cell-based assay further comprises a fourth component comprising a source of TG2. In some embodiments, the TG2 component is provided from the same, or different, source as any one of the above-mentioned components. The results from a non-limiting exemplary embodiment of a cell-based assay for measuring TGFβactivation demonstrating the inhibition of LTBP1-proTGFβ1 complex, LTBP3-proTGFβ1 complex, GARP-proTGFβ1 complex, or LRRC33-proTGFβ1 complex are disclosed herein.

Such cell-based assays may be modified or tailored in a number of ways depending on the TGFβ isoform being studied, the type of latent complex (e.g., presenting molecule), and the like. In some embodiments, a cell known to express integrin capable of activating TGFβ may be used as the source of integrin in the assay. Such cells typically include LN229 cells. Other suitable cells include SW480/β6 cells (e.g., clone 1E7). In some embodiments, the cell-line(s) may be modified to reduce or eliminate expression of one or more presenting molecules (e.g., through CRISPR-mediated gene ablation). For example, the cell-line may be a LTBP1 knock-out cell-line (e.g., CRISPR-mediated gene ablation by targeting exon 7). In other embodiments, the cell-line may be a LTBP3 knock-out cell-line. In other embodiments, the cell-line may be a GARP knock-out cell-line. In other embodiments, the cell-line may be a LRRC33 knock-out cell-line. In some embodiments, integrin-expressing cells may be co-transfected with a plasmid encoding a presenting molecule of interest (such as GARP, LRRC33, LTBP (e.g., LTBP1 or LTBP3), etc.) and a plasmid encoding a pro-form of the TGFβ isoform of interest (such as proTGFβ1). After transfection, the cells are incubated for sufficient time to allow for the expression of the transfected genes (e.g., about 24 hours), cells are washed, and incubated with serial dilutions of a test agent (e.g., an antibody). Then, a reporter cell line (e.g., CAGA12 cells) is added to the assay system, followed by appropriate incubation time to allow TGFβ signaling. After an incubation period (e.g., about 18-20 hours) following the addition of the test agent, signal/read-out (e.g., luciferase activity) is detected using suitable means (e.g., for luciferase-expressing reporter cell lines, the Bright-Glo reagent (Promega) can be used). In some embodiments, Luciferase fluorescence may be detected using a BioTek (Synergy H1) plate reader, with autogain settings.

In some embodiments, integrin-expressing cells may be co-transfected with a plasmid encoding a presenting molecule of interest (such as GARP, LRRC33, LTBP (e.g., LTBP1 or LTBP3), etc.) and a plasmid encoding a pro-form of the TGFβ isoform of interest (such as proTGFβ1). After transfection, the cells are incubated for sufficient time to allow for the expression of the transfected genes (e.g., about 24 hours), cells are washed, and incubated with serial dilutions of a test agent (e.g., an antibody). Then, a reporter cell line (e.g., CAGA12 cells) is added to the assay system, followed by appropriate incubation time to allow TGFβ signaling. After an incubation period (e.g., about 18-20 hours) following the addition of the test agent, signal/read-out (e.g., luciferase activity) is detected using suitable means (e.g., for luciferase-expressing reporter cell lines, the Bright-Go™ reagent (Promega®) can be used). In some embodiments, Luciferase fluorescence may be detected using a BioTek® (Synergy™ H1) plate reader, with autogain settings.

Data demonstrate that exemplary antibodies of the disclosure which are capable of selectively inhibiting the activation of TGFβ1.

A skilled artisan could readily adapt such assays to various suitable configurations. For instance, a variety of sources of TGFβ may be considered. In some embodiments, the source of TGFβ is a cell that expresses and deposits TGFβ (e.g., a primary cell, a propagated cell, an immortalized cell or cell line, etc.). In some embodiments, the source of TGFβ is purified and/or recombinant TGFβ immobilized in the assay system using suitable means. In some embodiments, TGFβ immobilized in the assay system is presented within an extracellular matrix (ECM) composition on the assay plate, with or without de-cellularization, which mimics fibroblast-originated TGFβ. In some embodiments, TGFβ is presented on the cell surface of a cell used in the assay. Additionally, a presenting molecule of choice may be included in the assay system to provide suitable latent-TGFβ complex. One of ordinary skill in the art can readily determine which presenting molecule(s) may be present or expressed in certain cells or cell types. Using such assay systems, relative changes in TGFβ activation in the presence or absence of a test agent (such as an antibody) may be readily measured to evaluate the effects of the test agent on TGFβ activation in vitro. Data from exemplary cell-based assays are provided in the Example section below.

### Nucleic Acids

In some embodiments, antibodies, antigen binding portions thereof, and/or compositions of the present disclosure may be encoded by nucleic acid molecules. Such nucleic acid molecules include, without limitation, DNA molecules, RNA molecules, polynucleotides, oligonucleotides, mRNA molecules, vectors, plasmids and the like. In some embodiments, the present disclosure may comprise cells programmed or generated to express nucleic acid molecules encoding compounds and/or compositions of the present disclosure. In some cases, nucleic acids of the disclosure include codon-optimized nucleic acids. Methods of generating codon-optimized nucleic acids are known in the art and may include, but are not limited to, those described in U.S. Pat. Nos. 5,786,464 and 6,114,148, the contents of each of which are herein incorporated by reference in their entirety.

The present disclosure is further illustrated by the following examples, which are not intended to be limiting in any way. The entire contents of all references, patents and published patent applications cited throughout this application, as well as the Figures, are hereby incorporated herein by reference.

### LIST OF CERTAIN EMBODIMENTS

Non-limiting embodiments of the present disclosure are listed below:

1. An antibody or an antigen-binding fragment thereof that binds each of the following antigen complexes with a KD of ≤10 nM, optionally ≤5 nM, as measured by a solution equilibrium titration-based assay:
i) hLTBP1-proTGFβ1;
ii) hLTBP3-proTGFβ1;
iii) hGARP-proTGFβ1; and,
iv) hLRRC33-proTGFβ1;
wherein the antibody or the fragment thereof is a fully human or humanized antibody or fragment thereof.

2. The antibody or the antigen-binding fragment according to embodiment 1, which binds each of the i) hLTBP1-proTGFβ1 and the ii) hLTBP3-proTGFβ1 complexes with a KD of ≤5 nM as measured by a solution equilibrium titration-based assay, wherein optionally, the antibody or the fragment binds each of the complexes with a KD of ≤1 nM as measured by a solution equilibrium titration-based assay

3. An antibody or an antigen-binding fragment thereof that binds each of the following antigen complexes with a KD of ≤200 pM, optionally ≤100 pM, as measured by a solution equilibrium titration-based assay:
i) hLTBP1-proTGFβ1;
ii) hLTBP3-proTGFβ1;
iii) hGARP-proTGFβ1; and,
iv) hLRRC33-proTGFβ1;
wherein the antibody or the fragment thereof is a fully human or humanized antibody or fragment thereof.

4. The antibody or the antigen-binding fragment according to any one of the preceding embodiments, which comprises CDR-H1, CDR-H2, CDR-H3, CDR-L1, CDR-L2, and CDR-L3, wherein:
the CDR-H1 has an amino acid sequence represented by FTF(X₁)(X₂)(X₃)(X₄)M(X₅), wherein optionally, X₁ is S, G or A; X₂ is S or F; X₃ is F or Y; X₄ is S or A; and/or, Xs is D, N or Y (SEQ ID NO: 1116);
the CDR-H2 has an amino acid sequence represented by YI(X₁)(X₂)(X₃)A(X₄)TIYYA(X₅)SVKG, wherein optionally, X₁ is S or H; X₂ is P or S; X₃ is S or D; X₄ is D or S; and/or, Xs is D or G (SEQ ID NO: 1117);
the CDR-H3 has an amino acid sequence represented by (X₁)R(X₂)(X₃)(X₄)D(X₅)GDML(X₆)P, wherein optionally, X₁ is A or V; X₂ is G or A; X₃ is V or T; X₄ is L or W; Xs is Y or M; and/or, X₆ is M or D (SEQ ID NO: 1118);
the CDR-L1 has an amino acid sequence QASQDITNYLN (SEQ ID NO: 1078), with optionally 1 or 2 amino acid changes;
the CDR-L2 has an amino acid sequence DASNLET (SEQ ID NO: 1079), with optionally 1 or 2 amino acid changes; and,
the CDR-L3 has an amino acid sequence QQADNHPPWT (SEQ ID NO: 1006), with optionally 1 or 2 amino acid changes.

5. The antibody or the antigen-binding fragment according to any one of the preceding embodiments, which comprises CDR-H1, CDR-H2, CDR-H3, CDR-L1, CDR-L2, and CDR-L3, wherein:
the CDR-H1 has an amino acid sequence FTFSSFSMD (SEQ ID NO: 1080), with optionally up to 4 amino acid changes, or, up to 2 amino acid changes;
the CDR-H2 has an amino acid sequence YISPSADTIYYADSVKG (SEQ ID NO: 1076), with optionally up to 4 amino acid changes;
the CDR-H3 has an amino acid sequence ARGVLDYGDMLMP (SEQ ID NO: 1003), with optionally up to 3 amino acid changes;
the CDR-L1 has an amino acid sequence QASQDITNYLN (SEQ ID NO: 1078), with optionally 1 or 2 amino acid changes;
the CDR-L2 has an amino acid sequence DASNLET (SEQ ID NO: 1079), with optionally 1 or 2 amino acid changes; and,
the CDR-L3 has an amino acid sequence QQADNHPPWT (SEQ ID NO: 1006), with optionally 1 or 2 amino acid changes.

6. The antibody or the antigen-binding fragment according to any one of the preceding embodiments, wherein the CDR-H1 comprises GFTFSSFS (SEQ ID NO: 1001); the CDR-H2 comprises ISPSADTI (SEQ ID NO: 1002); the CDR-H3 comprises ARGVLDYGDMLMP (SEQ ID NO: 1003); the CDR-L1 comprises QDITNY (SEQ ID NO: 1004); the CDR-L2 comprises DAS (SEQ ID NO: 1005); and, the CDR-L3 comprises QQADNHPPWT (SEQ ID NO: 1006).

7. The antibody or the antigen-binding fragment according to any one of the preceding embodiments, which binds an epitope that includes one or more amino acid residues of Latent Lasso, wherein optionally the epitope is a combinatorial epitope, wherein further optionally, the combinatorial epitope comprises one or more amino acid residues of Finger-1 and/or Finger-2 of the growth factor domain.

8. The antibody or the antigen-binding fragment of embodiment 7, wherein the epitope comprises one or more amino acid residues of KLRLASPPSQGEVPPGPLPEAVL (SEQ ID NO: 1142), and wherein optionally the epitope further comprises one or more amino acid residues of RKDLGWKWIHEPKGYHANF (SEQ ID NO: 1138) and/or VGRKPKVEQL (SEQ ID NO: 1141).

9. The antibody or the antigen-binding fragment of embodiment 8, wherein the epitope comprises one or more amino acid residues of KLRLASPPSQGEVPPGPLPEAVL (SEQ ID NO: 1142), and one or more amino acid residues of RKDLGWKWIHEPKGYHANF (SEQ ID NO: 1138).

10. The antibody or the antigen-binding fragment of embodiment 8, wherein the epitope comprises one or more amino acid residues of KLRLASPPSQGEVPPGPLPEAVL (SEQ ID NO: 1142) and one or more amino acid residues of VGRKPKVEQL (SEQ ID NO: 1141).

11. The antibody or the antigen-binding fragment of embodiment 7, wherein the epitope comprises one or more amino acid residues of KLRLASPPSQGEVPPGPLPEAVL (SEQ ID NO: 1142), one or more amino acid residues of RKDLGWKWIHEPKGYHANF (SEQ ID NO: 1138) and, one or more amino acid residues of VGRKPKVEQL (SEQ ID NO: 1141).

12. The antibody or the antigen-binding fragment according to any one of the preceding embodiments, wherein the antibody or the antigen-binding fragment is a fully human or humanized antibody or the antigen-binding fragment.

13. The antibody or the antigen-binding fragment according to any one of the preceding embodiments, wherein the antibody or the antigen-binding fragment cross-reacts with human and mouse proTGFβ1 complexes.

14. The antibody or the antigen-binding fragment according to any one of the preceding embodiments, wherein the antibody or the antigen-binding fragment is a human IgG4 or IgG1 subtype.

15. The antibody or the antigen-binding fragment according to any one of the preceding embodiments, wherein the antibody or the antigen-binding fragment comprises a backbone substitution of Ser to Pro that produces an IgG1-like hinge.

16. The antibody or the antigen-binding fragment according to any one of the preceding embodiments, which has an IC50 of ≤2 nM towards each of the following complexes as measured by a cell-based reporter assay.
i) hLTBP1-proTGFβ1;
ii) hLTBP3-proTGFβ1;
iii) hGARP-proTGFβ1; and,
iv) hLRRC33-proTGFβ1.

17. An isolated monoclonal antibody or a fragment thereof that specifically binds each of the following antigen with an affinity of <1 nM as measured by Biolayer Interferometry or surface plasmon resonance:
a) a human LTBP1-proTGFβ1 complex;
b) a human LTBP3-proTGFβ1 complex;
c) a human GARP-proTGFβ1 complex; and,
d) a human LRRC33-proTGFβ1 complex;
wherein the monoclonal antibody shows no more than a three-fold bias in affinity towards any one of the above complexes over the other complexes, and,
wherein the monoclonal antibody inhibits release of mature TGFβ1 growth factor from each of the proTGFβ1 complexes but not from proTGFβ2 or proTGFβ3 complexes.

18. An isolated monoclonal antibody or a fragment thereof that specifically binds a proTGFβ1 complex at a binding region having an amino acid sequence PGPLPEAV (SEQ ID NO: 1134) or a portion thereof,
characterized in that when bound to the proTGFβ1 complex in a solution, the antibody or the fragment protects the binding region from solvent exposure as determined by hydrogen-deuterium exchange mass spectrometry (HDX-MS); and,
wherein the antibody or the fragment specifically binds each of the following complexes: LTBP1-proTGFβ1, LTBP3-proTGFβ1, GARP-proTGFβ1, and LRRC33-proTGFβ1, with an affinity of <5 nM as measured by Biolayer Interferometry or surface plasmon resonance.

19. An isolated monoclonal antibody or a fragment thereof that specifically binds a proTGFβ1 complex at a binding region having an amino acid sequence LVKRKRIEA (SEQ ID NO: 1132) or a portion thereof,
characterized in that when bound to the proTGFβ1 complex in a solution, the antibody or the fragment protects the binding region from solvent exposure as determined by hydrogen-deuterium exchange mass spectrometry (HDX-MS); and,
wherein the antibody or the fragment specifically binds each of the following complexes: LTBP1-proTGFβ1, LTBP3-proTGFβ1, GARP-proTGFβ1, and LRRC33-proTGFβ1, with an affinity of <5 nM as measured by Biolayer Interferometry or surface plasmon resonance.

20. An isolated monoclonal antibody or a fragment thereof that specifically binds a proTGFβ1 complex at
i) a first binding region comprising at least a portion of Latency Lasso (SEQ ID NO: 1126); and
ii) a second binding region comprising at least a portion of Finger-1 (SEQ ID NO: 1124);
characterized in that when bound to the proTGFβ1 complex in a solution, the antibody or the fragment protects the binding regions from solvent exposure as determined by hydrogen-deuterium exchange mass spectrometry (HDX-MS).

21. The antibody or the fragment according to embodiment 45, wherein the first binding region comprises PGPLPEAV (SEQ ID NO: 1134) or a portion thereof and the second binding region comprises RKDLGWKW (SEQ ID NO: 1143) or a portion thereof.

22. The antibody or the fragment according to any one of the preceding embodiments, wherein the antibody is a context-independent antibody such that it binds matrix-associated proTGFβ1 complexes and cell-associated proTGFβ1 complexes with less than five-fold bias in affinity, as measured by Biolayer Interferometry or surface plasmon resonance.

23. The antibody or the fragment according to any one of embodiments 43-47, which specifically binds each of the following complexes: mLTBP1-proTGFβ1, mLTBP3-proTGFβ1, mGARP-proTGFβ1, and mLRRC33-proTGFβ1, with an affinity of <1 nM.

24. The antibody or the fragment according to any one of the preceding embodiments that binds the proTGFβ1 complex at one or more of the following binding regions or a portion thereof:

(SEQ ID NO: 1132)
LVKRKRIEA;
(SEQ ID NO: 1126)
LASPPSQGEVPPGPL;
(SEQ ID NO: 1134)
PGPLPEAV;
(SEQ ID NO: 1135)
LALYNSTR;
(SEQ ID NO: 1136)
REAVPEPVL;
(SEQ ID NO: 1137)
YQKYSNNSWR;
(SEQ ID NO: 1144)
RKDLGWKWIHE;
(SEQ ID NO: 1145)
HEPKGYHANF;
(SEQ ID NO: 1139)
LGPCPYIWS;
(SEQ ID NO: 1140)
ALEPLPIV;
and,
(SEQ ID NO: 1141)
VGRKPKVEQL.

25. The antibody or the fragment according to any one of the preceding embodiments, having a CDR sequence selected from the group consisting of:

(SEQ ID NO: 1001)
GFTFSSFS
(SEQ ID NO: 1002)
ISPSADTI
(SEQ ID NO: 1003)
ARGVLDYGDMLMP
(SEQ ID NO: 1004)
QDITNY
(SEQ ID NO: 1005)
DAS
and
(SEQ ID NO: 1006)
QQADNHPPWT

26. The antibody according to embodiment 25, which comprises all of the CDRs.

27. An antibody or an antigen-binding fragment thereof that binds each of the following antigen:
hLTBP1-proTGFβ1
hLTBP3-proTGFβ1
hGARP-proTGFβ1; and,
hLRRC33-proTGFβ1;
wherein the antibody or the fragment binds each of the hLTBP1-proTGFβ1 and hLTBP3-proTGFβ1 with a K_{D} of ≤1 nM as measured by a solution equilibrium titration-based assay;
wherein the antibody or the fragment binds an epitope comprising one or more amino acid residues of LRLASPPSQGEVPPGPLPEAV (SEQ ID NO: 1146), and optionally the epitope further comprises one or more amino acid residues of RKDLGWKWIHEPKGYHANF (SEQ ID NO: 1138).

28. The antibody or the antigen-binding fragment according to any one of the preceding embodiments,
wherein the antibody or the fragment binds each of LTBP1-proTGFβ1 and LTBP3-proTGFβ1 with an affinity of ≤1 nM; and
wherein the antibody or the fragment binds matrix-associated proTGFβ1 complexes with at least 10-fold higher affinities than cell-associated proTGFβ1 complexes.

29. The antibody or the antigen-binding fragment according to the preceding embodiment, wherein the in vitro binding IC₅₀ is ≤5 nM, wherein optionally the IC₅₀ is ≤1 nM.

30. The antibody or the antigen-binding fragment according to any one of the preceding embodiments, wherein the antibody or the antigen-binding fragment is capable of inhibiting integrin-dependent activation of TGFβ1.

31. The antibody or the antigen-binding fragment according to any one of the preceding embodiments, wherein the antibody or the antigen-binding fragment is capable of inhibiting protease-dependent activation of TGFβ1.

32. The antibody or the antigen-binding fragment according to any one of the preceding embodiments, wherein the antibody or the antigen-binding fragment is capable of inhibiting integrin-dependent activation of TGFβ1 and protease-dependent activation of TGFβ1.

33. The antibody or the fragment thereof according any one of the preceding embodiments, which does not specifically bind proTGFβ2 or proTGFβ3.

34. The antibody or the fragment thereof according any one of the preceding embodiments, which does not specifically bind free TGFβ1 growth factor which is not in association with a proTGFβ1 complex.

35. An antibody or an antigen-binding fragment thereof that cross-blocks with the antibody or the fragment according any one of the preceding embodiments.

36. A kit comprising the antibody or the fragment according to any one of the preceding embodiments.

37. A composition comprising the antibody or the fragment according to any one of the preceding embodiments, and a pharmaceutically acceptable excipient.

38. The composition of embodiment 37 for use in therapy in the treatment of a TGFβ-related indication in a subject.

39. The composition for use according to embodiment 38, wherein the TGFβ-related indication is cancer, myelofibrosis, stem cell disorder, and/or fibrotic disorder.

40. The composition for use according to embodiment 38, wherein the TGFβ-related indication is selected from the following:
i) disease in which TGFβ1 is overexpressed or TGFβ1 signaling is dysregulated;
ii) disease associated with abnormal stem cell differentiation or repopulation, which is optionally:
   a) stem cell/progenitor cell differentiation/reconstitution is halted or perturbed due to a disease or induced as a side effect of a therapy/mediation;
   b) patients are on a therapy or mediation that causes healthy cells to be killed or depleted;
   c) patients may benefit from increased stem cell/progenitor cell differentiation/reconstitution;
   d) disease is associated with abnormal stem cell differentiation or reconstitution
iii) conditions involving hematopoietic dysregulation, such as treatment-induced hematopoietic dysregulation;
iv) diseases with aberrant gene expression of one or more genes selected from the group consisting of: Serpine 1 (encoding PAI-1), MCP-1 (also known as CCL2), CCL3, Col1a1, Col3a1, FN1, TGFB1, CTGF, ACTA2 (encoding α-SMA), ITGA11, SNAI1, MMP2, MMP9, TIMP1, FOXP3, CDH1 (E cadherin), and, CDH2;
v) diseases involving proteases
vi) diseases Involving mesenchymal transition, such as Epithelial-to-Mesenchymal Transition (EMT) and/or Endothelial-to-Mesenchymal Transition (EndMT);
vii) diseases Involving immunosuppression, wherein optionally the immunosuppression comprises increased immunosuppressive cells at disease site, wherein further optionally the immunosuppressive cells are M2 macrophages and/or MDSCs;
viii) diseases involving Matrix Stiffening and Remodeling; optionally comprising ECM stiffness;
ix) organ fibrosis, optionally advanced organ fibrosis
x) primary and secondary myelofibrosis
xi) Malignancies/cancer
   a) solid tumor, optionally advanced solid tumor or metastatic tumor;
   b) blood cancer.

41. The composition for use according to embodiment 40, wherein the cancer comprises a solid tumor, or, wherein the cancer is a blood cancer.

42. The composition for use according to embodiment 41, wherein the solid tumor is poorly responsive to a cancer therapy, wherein optionally the cancer therapy is a checkpoint inhibitor therapy, cancer vaccine, chemotherapy, radiation therapy, oncolytic virus therapy, IDO inhibitor therapy, and/or an engineered immune cell therapy.

43. The composition for use according to embodiment 41, wherein the solid tumor is an immune-excluded tumor.

44. The composition for use according to embodiment 41, wherein the solid tumor comprises Tregs, intratumoral M2 macrophages and/or MDSCs.

45. The composition for use according to embodiment 41, wherein the solid tumor comprises stroma enriched with CAFs and/or myofibroblasts.

46. The composition for use according to embodiment 41, wherein the subject is receiving or is a candidate for receiving a cancer therapy selected from the group consisting of: chemotherapy, radiation therapy, CAR-T, cancer vaccine, oncolytic viral therapy and checkpoint inhibitor therapy.

47. The composition for use according to embodiment 41, wherein the cancer is characterized by acquired resistance or primary resistance to the cancer therapy.

48. The composition for use according to any one of embodiment 38-47, wherein the treatment of cancer comprises administration of a therapeutically effective amount of the composition to reduce the growth of the solid tumor, wherein optionally the administration of the composition increases survival.

49. The composition for use according to any one of embodiment 38-48, wherein the treatment comprises administration of the composition at a dose ranging between 1-30 mg/kg.

50. A method for selecting a subject likely to respond to a TGFβ1 inhibition therapy, comprising the step of:
identifying a subject diagnosed with cancer, wherein, i) the cancer is a type of cancer known to be susceptible for resistance to a cancer therapy, and/or, ii) the subject is resistant to a cancer therapy, wherein optionally the subject is a primary non-responder to the cancer therapy;
wherein optionally the cancer therapy is chemotherapy, radiation therapy and/or immune checkpoint inhibition therapy; and,
selecting the subject as a candidate for a TGFβ1 inhibition therapy.

51. A method for treating cancer, the method comprising steps of:
i) selecting a patient diagnosed with cancer comprising a solid tumor, optionally wherein the solid tumor is or is suspected to be an immune excluded tumor;
ii) administering to the patient the antibody or the fragment according to any one of embodiments 1-10 in an amount effective to treat the cancer,
wherein (a) the patient has received, or is a candidate for receiving a cancer therapy selected from the group consisting of: immune checkpoint inhibition therapies (CBTs), chemotherapies, radiation therapies, engineered immune cell therapies, and cancer vaccine therapies; or, (b) the patient has a cancer with statistically low primary response rates, and wherein the patient has not received a CBT.

52. The method of embodiment 51, wherein the immune checkpoint inhibitor is a PD-1 inhibitor or a PD-L1 inhibitor.

53. The method of embodiment 52, wherein the selection step (i) comprises detection of immune cells or one or more markers thereof.

54. The method of embodiment 53, wherein the detection comprises a tumor biopsy analysis, serum marker analysis, and/or in vivo imaging.

55. The method of embodiments 53 or 54, wherein the immune cells are selected from the group consisting of: cytotoxic T lymphocytes, regulatory T cells, MDSCs, tumor-associated macrophages, NK cells, dendritic cells, and neutrophils.

56. The method of any one of embodiments 53-55, wherein the immune cell marker is selected from the group consisting of: CD8, CD3, CD4, CD11b, CD163, CD68, CD14, CD34, CD25, CD47.

57. The method of embodiment 54, wherein the in vivo imaging comprises T cell tracking.

58. The method of embodiment 54 or 57, wherein the in vivo imaging comprises the use of PET-SPECT, MRI and/or optical fluorescence/bioluminescence.

59. The method of embodiment 57 or 58, wherein the in vivo imaging comprises direct or indirect labeling of immune cells or antibody that binds a cell-surface marker of immune cells.

60. The method of any one of embodiments 54-59, wherein the in vivo imaging comprises the use of a tracer.

61. The method of embodiment 60, wherein the tracer is a radioisotope.

62. The method of embodiment 61, wherein the radioisotope is a positron-emitting isotope.

63. The method of embodiment 62, wherein the radioisotope is selected from the group consisting of: ¹⁸F, ¹¹C, ¹³N, ¹⁵O, ⁶⁸Ga, ¹⁷⁷Lu, ¹⁸F and ⁸⁹Zr.

64. The method of any one of embodiments 54-63, wherein the in vivo imaging comprise the use of labeled antibodies in immune-PET.

65. The method of any one of embodiments 54-64, wherein the in vivo imaging is performed for monitoring a therapeutic response to the TGFβ1 inhibition therapy in the subject.

66. The method of embodiment 65, wherein the therapeutic response comprises conversion of an immune excluded tumor into an inflamed tumor and/or a reduction in the level of circulating MDSCs (such as g-MDSCs).

67. A method of identifying an isoform-selective inhibitor of TGFβ1 activation for therapeutic use, the method comprising the steps of:
i) selecting a pool of antibodies or antigen-binding fragments capable of binding each of: hLTBP1-proTGFβ1; hLTBP3-proTGFβ1; hGARP-proTGFβ1; and, hLRRC33-proTGFβ1 in vitro with a KD of ≤10 nM as measured by a solution equilibrium titration-based assay;
ii) selecting a pool of antibodies or antigen-binding fragments capable of inhibiting TGFβ activation, optionally in a cell-based assay;
iii) testing one or more antibodies or antigen-binding fragments thereof from steps i) and ii) in an in vivo efficacy study;
iv) testing one or more antibodies or antigen-binding fragments thereof from steps i)-iii) in an in vivo toxicology/safety study; and,
v) identifying one or more antibodies or antigen-binding fragments from steps i)-iv), wherein the antibodies or the fragments show efficacious doses determined in the in vivo efficacy study that are below a NOAEL determined in the in vivo toxicology/safety study.

68. Use of the antibody or the fragment according to any one of embodiments 1-35 in the manufacture of a medicament for the treatment of a TGFβ1 indication.

69. The use according to embodiment 68, further comprising a step of sterile filtration of a formulation comprising the antibody or the fragment.

70. The use according to embodiment 68 or 69, further comprising a step of filling and/or packaging into a vial or a syringe.

71. A method for making a pharmaceutical composition comprising an isoform-selective TGFβ1 inhibitor, the method comprising:
i) providing an antibody capable of binding each of hLTBP1-proTGFβ1, hLTBP3-proTGFβ1, hGARP-proTGFβ1 and hLRRC33-proTGFβ1 with a KD of 1 nM or less,
ii) carrying out an in vivo efficacy study wherein the antibody of step (i) is administered to a preclinical model to determine effective amounts,
iii) carrying out a toxicology study using an animal model known to be sensitive to TGFβ inhibition, to determine amounts at which undesirable toxicities are observed;
iv) determining or confirming a sufficient therapeutic window based on steps (ii) and (iii); and,
v) manufacturing a pharmaceutical composition comprising the antibody.

72. A method of manufacturing the antibody or the fragment according to any one of embodiments 1-35, the method comprising steps of:
i) providing an antigen that comprises a proTGFβ1 complex, optionally comprising at least two of: LTBP1, LTBP3, GARP, LRRC33 or a fragment thereof,
ii) selecting for a pool of antibodies or fragments for ability to bind the antigen of step (i);
iii) optionally removing antibodies or fragments from the pool that show undesirable binding profiles;
iv) selecting for a pool of antibodies or fragments selected from step(s) (ii) and/or (iii) for ability to inhibit TGFβ1;
v) optionally generating a fully human or humanized antibody or fragment of an antibody, antibodies or fragments selected from step (iv) so as to provide a human or humanized inhibitor;
vi) carrying out in vitro binding assay to determine affinities for LTBP1-proTGFβ1, LTBP3-proTGFβ1, GARP-proTGFβ1 and LRRC33-proTGFβ1,
vii) carrying out functional assay to determine or confirm activity of the inhibitor towards TGFβ1 and optionally TGFβ2 and/or TGFβ3.

73. The method of embodiment 72, further comprising a step of evaluating a candidate antibody or a fragment thereof in an in vivo efficacy study and in vivo toxicology study in a preclinical animal model, thereby determining effective amounts shown to be both efficacious and safe or tolerable.

74. The method of embodiment 72 or 73, further comprising a step of formulating into a pharmaceutical composition.

75. The composition according to embodiment 74 for therapeutic use in the treatment of fibrosis in a human subject.

76. The composition according to embodiment 74 for therapeutic use in the treatment of myelofibrosis in a human subject.

77. The composition according to embodiment 74 for therapeutic use in the treatment of cancer in a human subject.

78. The composition for use according to embodiment 77, wherein the cancer comprises a solid tumor.

79. The composition for use according to embodiment 78, wherein the solid tumor is a locally advanced solid tumor.

80. The composition for use according to any one of embodiments 77-79, wherein the cancer is poorly responsive to a cancer therapy, wherein optionally the cancer therapy is a checkpoint inhibitor therapy, cancer vaccine, chemotherapy, radiation therapy, IDO inhibitor therapy, and/or an engineered immune cell therapy.

81. The composition for use according to embodiment 80, wherein the cancer is characterized by acquired resistance or primary resistance.

82. The composition for use according to embodiment 81, wherein the tumor is characterized by immune exclusion.

83. The composition for use according to any one of embodiments 78-82, wherein the tumor comprises intratumoral M2 macrophages and/or MDSCs.

84. The composition for use according to any one of embodiments 78-82, wherein the tumor comprises stroma enriched with CAFs.

85. The composition for use according to embodiment 80, wherein the subject is receiving or is a candidate for receiving a cancer therapy selected from the group consisting of: chemotherapy, radiation therapy, CAR-T, cancer vaccine, oncolytic viral therapy and checkpoint inhibitor therapy.

86. The composition for use according to any one of embodiments, wherein the subject is further treated with a TGFβ3 inhibitor.

87. The composition for use according to embodiment 70, wherein the subject has TGFβ1-positive and TGFβ3-positive cancer and wherein the subject has been, is on or is a candidate for receiving a checkpoint inhibitor therapy.

88. The composition for use according to any one of embodiments 77-84, wherein the subject is not a candidate for undergoing surgical resection of the tumor.

89. The composition according to embodiment 37 for use in the enhancement of host immunity in a human subject,
wherein the subject has cancer, and
wherein the immune responses comprise anti-cancer immunity.

90. The composition for use according to embodiment 89 wherein the enhancement of host immunity includes reducing immune-exclusion from a tumor or promoting immune cell infiltrates into a tumor.

91. The composition for use according to embodiment 89 wherein the enhancement of host immunity includes inhibiting plasmin-dependent activation of TGFβ1.

92. The composition for use according to embodiment 37, wherein the subject is at risk of developing a cytokine storm.

93. The composition for use according to embodiment 37, wherein the subject is receiving or a candidate for receiving an engineered immune cell therapy.

94. The composition for use according to embodiment 37, wherein the subject is receiving or is a candidate for receiving a cancer vaccine.

95. The composition for use according to any one of embodiments 76-94, wherein the subject is receiving or is a candidate for receiving an immune checkpoint inhibitor therapy, wherein optionally the subject is poorly responsive to the immune checkpoint inhibitor therapy.

96. The composition according to embodiment 37 for use in the prevention of a cytokine release syndrome, (e.g., cytokine storm or sepsis) in a human subject, wherein optionally the subject is suffering from an infection or MS.

97. The composition according to embodiment 37 for use in a method for inhibiting plasmin-dependent activation of TGFβ1 in a subject.

98. A method for treating a TGFβ1 indication in a subject, the method comprising a step of administering to the subject a therapeutically effective amount of an isoform-selective TGFβ1 inhibitor to treat the indication, wherein, the isoform-selective TGFβ1 inhibitor is a monoclonal antibody that specifically binds each of hLTBP1-proTGFβ1; hLTBP3-proTGFβ1; hGARP-proTGFβ1; and, hLRRC33-proTGFβ1 with a KD of ≤10 nM as measured by solution equilibrium titration.

99. The method of embodiment 98, wherein the antibody binds each of the hLTBP1-proTGFβ1 and hLTBP3-proTGFβ1 with a KD of ≤1 nM as measured by solution equilibrium titration, wherein optionally, the antibody binds each of the hLTBP1-proTGFβ1; hLTBP3-proTGFβ1; hGARP-proTGFβ1; and, hLRRC33-proTGFβ1 complexes with a KD of ≤1 nM.

100. The method of embodiment 98 or 99, wherein the antibody binds Latency Lasso or a portion thereof.

101. The method of embodiment 100, wherein the antibody further binds Finger-1, Finger-2, or a portion(s) thereof.

102. The method of any one of embodiments 98-101, wherein the TGFβ1 indication is a proliferative disorder selected from cancer and myeloproliferative disorders.

103. The method of embodiment 102, wherein the subject is a poor responder of a cancer therapy, wherein optionally the cancer therapy comprises a checkpoint inhibition therapy, chemotherapy and/or radiation therapy.

104. The method of embodiment 102, wherein the subject is further treated with a cancer therapy in conjunction with the isoform-selective TGFβ1 inhibitor.

Additional non-limiting embodiments of the present disclosure are provided below.

1. A method of treating cancer in a subject, wherein the treatment comprises administering to the subject a TGFβ inhibitor in an amount sufficient to reduce circulating MDSC levels.

2. The method of embodiment 1, wherein the reduced circulating MDSCs are G-MDSCs.

3. The method of embodiment 1 or 2, wherein the G-MDSCs express one or more of CD11b, CD33, CD15, LOX-1, CD66b, and HLA-DR^{lo/-}.

4. The method of any one of embodiments 1-3, wherein the treatment further comprises administering a cancer therapy.

5. The method of any one of embodiments 1-4, wherein the TGFβ inhibitor and the cancer therapy are administered concurrently (e.g., simultaneously), separately, or sequentially.

6. The method of embodiment 4 or 5, comprising determining whether a subject has a reduction in circulating MDSC levels following administration of the TGFβ inhibitor, and administering the cancer therapy if MDSC levels have been reduced.

7. The method of any one of embodiments 1-6, wherein the cancer therapy comprises a checkpoint inhibitor therapy, optionally an agent targeting PD-1 or PD-L1, optionally an anti-PD-1 or anti-PD-L1 antibody.

8. A method of predicting therapeutic efficacy in a subject having cancer, comprising:
(i) determining circulating MDSC levels in the subject prior to administering a TGFβ inhibitor (alone or in combination with a cancer therapy);
(ii) administering to the subject a therapeutically effective amount of the TGFβ inhibitor (alone or in combination with a cancer therapy); and
iii) determining circulating MDSC levels in the subject after the administration, wherein a reduction in circulating MDSC levels after administration, as compared to circulating MDSC levels before administration, predicts pharmacological effects.

9. A method of treating cancer in a subject, comprising the steps of:
(i) determining circulating MDSC levels in the subject prior to administering a TGFβ inhibitor;
(ii) administering to the subject a first therapeutically effective dose of the TGFβ inhibitor;
(iii) determining circulating MDSC levels in the subject after administering the TGFβ inhibitor;
(iv) administering to the subject a second therapeutically effective dose of the TGFβ inhibitor if the circulating MDSC levels measured after administering the first therapeutically effective dose of the TGFβ inhibitor are reduced as compared to the circulating MDSC levels measured prior to administering the first therapeutically effective dose of the TGFβ1 inhibitor.

10. The method of embodiment 8 or embodiment 9, comprising administering a checkpoint inhibitor therapy concurrently (e.g., simultaneously), separately, or sequentially with the TGFβ inhibitor.

11. A method of treating cancer in a subject, comprising the steps of:
(i) determining circulating MDSC levels in the subject prior to administering a combination therapy comprising a therapeutically effective amount of a TGFβ inhibitor and a therapeutically effective amount of a checkpoint inhibitor therapy;
(ii) administering to the subject the combination therapy;
(iii) determining circulating MDSC levels in the subject after administering the combination therapy;
(iv) continuing the combination therapy if the circulating MDSC levels measured after administering the first therapeutically effective dose of the combination therapy are reduced as compared to the circulating MDSC levels measured prior to administering the first therapeutically effective dose.

12. The method of embodiment 11, wherein the combination therapy comprises administering the TGFβinhibitor concurrently (e.g., simultaneously), separately, or sequentially with the checkpoint inhibitor therapy.

13. A method of treating advanced cancer in a human subject, the method comprising the steps of
i) selecting a subject with advanced cancer comprising a locally advanced tumor and/or metastatic cancer with primary resistance to a checkpoint inhibitor therapy,
ii) administering a TGFβ inhibitor; and,
ii) administering to the subject a checkpoint inhibitor therapy.

14 The method of embodiment 13, wherein the checkpoint inhibitor therapy is administered concurrently (e.g., simultaneously), separately, or sequentially with the TGFβ inhibitor.

15. A method for treating advanced cancer in a human subject, the method comprising the steps of
i) selecting a subject with advanced cancer comprising a locally advanced tumor and/or metastatic cancer with primary resistance to a CPI therapy, wherein the subject has received a TGFβ inhibitor which is a TGFβ1-selective inhibitor or a TGFβ inhibitor that does not inhibit TGFβ3; and,
ii) administering to the subject a CPI therapy, optionally in conjunction with the TGFβ inhibitor.

16 The method of any one of embodiments 13-15, further comprising measuring the levels of circulating MDSC levels before and after administering the treatment, wherein a reduction in circulating MDSC levels is indicative of a treatment response.

17. The method of embodiment 16, further comprising continuing the treatment if a reduction in circulating MDSC levels is determined.

18. The method of any one of embodiments 8-17, wherein the reduced circulating MDSCs are G-MDSCs.

19. The method of embodiment 18, wherein the G-MDSCs express one or more of CD11b, CD33, CD15, LOX-1, CD66b, and HLA-DR^{lo/-}.

20. The method of any one of embodiments 1-19, wherein the circulating MDSC levels are determined from whole blood or a blood component collected from the subject.

21 The method of any one of embodiments 1-20, wherein the treatment reduces circulating MDSC levels by at least 10%, optionally by at least 15%, 20%, 25%, or more.

22. The method of any one of embodiments 1-21, wherein the TGFβ inhibitor is a TGFβ1 inhibitor, optionally a TGFβ1-specific inhibitor.

23 The method of any one of embodiments 1-21, wherein the subject has circulating MDSC levels at least 2-fold above circulating MDSC levels in a healthy subject prior to a treatment.

24 A method of selecting a subject for treatment, wherein the subject has circulating MDSC levels at least 2-fold above circulating MDSC levels in a healthy subject prior to treatment.

25 The method of 24, wherein the subject has or is suspected of having cancer.

26 A method of treating a subject for cancer, wherein the subject has circulating MDSC levels at least 2-fold above circulating MDSC levels in a healthy subject prior to treatment, comprising administering to the subject a TGFβ inhibitor in an amount sufficient to reduce circulating MDSC levels.

27 The method of any one of embodiments 1-26, wherein the level of circulating MDSC cells is determined within 3-6 weeks, e.g., within or at about 3 weeks, following administration of a TGFβ inhibitor.

28 The method of any one of embodiments 1-27, wherein the level of circulating MDSC cells is determined within 2 weeks, e.g., 10 days, following administration of the TGFβ inhibitor.

29. The method of any one of embodiments 1-28, further comprising the steps of:
(i) determining the levels of tumor-associated immune cells in the subject prior to administering a treatment;
(ii) administering the treatment to the subject; and
(iii) determining the levels of tumor-associated immune cells in the subject after administering the treatment;
   wherein a change in the level of one or more tumor-associated immune cell populations after inhibitor administration, as compared to the levels of tumor-associated immune cells before administration, indicates therapeutic efficacy.

30. The method of embodiment 29, wherein a change in levels of tumor-associated immune cells in step (iii) indicates reduction or reversal of immune suppression in the cancer.

31. The method of embodiment 29 or 30, wherein the tumor-associated immune cells comprise CD8+ T cells and/or tumor-associated macrophages (TAMs).

32. The method of embodiments 29-31, wherein the change in the levels of tumor-associated immune cells comprises at least a 10%, optionally at least a 15%, 20%, 25%, or more, increase in CD8+ T cell levels.

33. The method of embodiments 29-32, wherein the change in the levels of tumor-associated immune cells comprises at least a 10%, optionally at least a 15%, 20%, 25%, or more, increase in the level of TAMs.

34. The method of any one of embodiments 29-33, wherein the levels of tumor-associated immune cells are determined in a sample collected from the subject by immunohistochemistry analysis.

35. The method of any one of embodiments 29-34, wherein the levels of tumor-associated immune cells are determined by in vivo imaging.

36. The method of any one of embodiments 29-34, wherein the sample is a tumor biopsy sample.

37. The method of any one of embodiments 29-36, further comprising continuing to administer the treatment if a change in the level of one or more tumor-associated immune cell populations is detected.

38. The method of any one of embodiments 1-37, further comprising the steps of:
(i) determining the levels of circulating latent TGFβ in the subject prior to administering a treatment;
(ii) administering the treatment to the subject; and
(iii) determining the levels of circulating latent TGFβ in the subject after administering the treatment; and
wherein an increase in circulating latent TGFβ after inhibitor administration, as compared to circulating latent TGFβ before administration, indicates therapeutic efficacy.

39. The method of embodiment 38, further comprising continuing to administer the treatment if a change in the level of circulating latent TGFβ is detected.

40 The method of 38 or 39, wherein the level of circulating latent TGFβ is determined in a sample obtained from the subject.

41 The method of 40, wherein the sample is a whole blood sample or a blood component.

42 The method of any one of embodiments 39-41, wherein the circulating latent TGFβ is circulating latent TGFβ1.

43. A method of treating cancer, comprising administering to a subject a TGFβ inhibitor in a therapeutically effective amount that does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1β), and chemokine C-C motif ligand 2 (CCL2)/monocyte chemoattractant protein 1 (MCP-1).

44. A method for identifying whether a TGFβ inhibitor will be tolerated in a patient, comprising contacting a cell culture or fluid sample with the TGFβ inhibitor and determining whether it causes a significant release of one or more cytokines selected from interferon gamma (IFNγ), interleukin 2 (IL-2), interleukin 6 (IL-6), tumor necrosis factor alpha (TNFα), interleukin 1 beta (IL-1β) and chemokine C-C motif ligand 2 (CCL2)/monocyte chemoattractant protein 1 (MCP-1), wherein a significant release indicates the TGFβ inhibitor will not be well tolerated.

45. The method of embodiment 43 or 44, wherein cytokine release is assessed in an in vitro cytokine release assay, optionally an assay in peripheral blood mononuclear cells (PBMCs) or whole blood, optionally wherein the PBMCs or whole blood are obtained from the subject prior to administering a TGFβ inhibitor therapy.

46. The method of any one of embodiments 43-45, wherein cytokine release is assessed in an in vitro cytokine release assay of peripheral blood mononuclear cells (PBMCs) or whole blood obtained from a healthy subject.

47. The method of embodiment 45 or 46, wherein the cytokine release assay comprises a soluble phase and/or a solid phase assay format.

48. The method of any one of embodiments 45-47, wherein the cytokine release assay comprises: i) a solid phase assay, ii) a high-density PBMC pre-culture assay, and/or iii) a PBL-HUVEC co-culture assay.

49. The method of any one of embodiments 45-48, wherein the cytokine release assay comprises a multiplex array, e.g., a Luminex® array system.

50. The method of any one of embodiments 45-49, wherein the cytokine release assay comprises comparing cytokine release from a TGFβ inhibitor to release from one or more control antibodies selected from an anti-CD3 antibody and an anti-CD28 antibody, optionally wherein the CD28 antibody optionally comprises TGN1412.

51. The method of any one of embodiments 43-50, wherein the TGFβ inhibitor does not induce more than a 10-fold increase in IL-6 levels, optionally less than a 2-fold, 4-fold, 6-fold, or 8-fold increase in IL-6 levels, as compared to levels in the absence of the inhibitor or in the presence of a control antibody.

52. The method of any one of embodiments 43-51, wherein the TGFβ inhibitor does not induce more than a 10-fold increase in IFNγ levels, optionally less than a 2-fold, 4-fold, 6-fold, or 8-fold increase in IFNγ levels, as compared to levels in the absence of the inhibitor or in the presence of a control antibody.

53. The method of embodiments 43-52, wherein the TGFβ inhibitor does not induce more than a 10-fold increase in TNFα levels, optionally less than a 2-fold, 4-fold, 6-fold, or 8-fold increase in TNFα levels, as compared to levels in the absence of the inhibitor or in the presence of a control antibody.

54. The method of embodiment any one of embodiments 43-53, wherein the TGFβ inhibitor is administered in a therapeutically effective amount that does not cause a significant release of one or more cytokines in an animal model comprising a non-human primate.

55. The method of embodiments 43-54, wherein the therapeutically effective amount of the TGFβ inhibitor is an amount sufficient to reduce circulating MDSC levels.

56. The method of embodiment 55, wherein the reduced MDSCs are G-MDSCs.

57. The method of embodiment 56, wherein the G-MDSCs express one or more of CD11 b, CD33, CD15, LOX-1, CD66b, and HLA-DR^{lo/-}.

58. The method of any one of embodiments 55-57, wherein the circulating MDSC levels are determined from whole blood or a blood component collected from the subject.

59. The method of any one of embodiments 43-58, wherein the TGFβ inhibitor is a TGFβ1 inhibitor, optionally a TGFβ1-specific inhibitor, e.g., Ab6.

60. A TGFβ inhibitor for use in the treatment of cancer in a subject, wherein the treatment comprises administration of a dose of said TGFβ inhibitor to the subject having cancer, wherein said TGFβ inhibitor does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1β) and chemokine C-C motif ligand 2 (CCL2)/monocyte chemoattractant protein 1 (MCP-1).

61. A combination therapy comprising a dose of a TGFβ inhibitor and a cancer therapy agent for use in the treatment of cancer, wherein the treatment comprises simultaneous, separate or sequential administration to a subject of a dose of the TGFβ inhibitor and the cancer therapy agent, wherein said TGFβ inhibitor does not cause a significant release of one or more cytokines selected from interferon gamma (IFNγ), interleukin 2 (IL-2), interleukin 6 (IL-6), tumor necrosis factor alpha (TNFa), interleukin 1 beta (IL-1β) and chemokine C-C motif ligand 2 (CCL2)/monocyte chemoattractant protein 1 (MCP-1).

62. The TGFβ inhibitor for use according to embodiment 60 or the combination therapy for use according to embodiment 61, wherein the TGFβ inhibitor is administered in a therapeutically effective amount that does not cause a significant release of one or more cytokines in an animal model comprising a non-human primate.

63. The TGFβ inhibitor for use according to embodiment 60 or 62, or the combination therapy for use according to embodiment 61, wherein the TGFβ inhibitor is administered in a therapeutically effective amount that is sufficient to reduce circulating MDSC levels.

64 The TGFβ inhibitor for use or the combination therapy for use according to embodiment 63, wherein the reduced MDSCs are G-MDSCs.

65 The TGFβ inhibitor for use or the combination therapy for use according to embodiment 64, wherein the G-MDSCs express one or more of CD11b, CD33, CD15, LOX-1, CD66b, and HLA-DR^{lo/-}.

66 The TGFβ inhibitor for use or the combination therapy for use according to any one of embodiments 63-65, wherein the circulating MDSC levels are determined from whole blood or a blood component collected from the subject.

67. The method of embodiment 44 or any embodiment dependent thereon, the TGFβ inhibitor for use according to embodiment 60 or any embodiment dependent thereon, or the combination therapy for use according to embodiment 61 or any embodiment dependent thereon, wherein the TGFβ inhibitor is a TGFβ1 inhibitor, optionally a TGFβ1-specific inhibitor.

68. The TGFβ inhibitor for use according to embodiment 60 or any embodiment dependent thereon, or the combination therapy for use according to embodiment 61 or any embodiment dependent thereon, wherein the TGFβ inhibitor does not cause significant release of one or more cytokines as determined by the method of embodiment 44 or any embodiment dependent thereon.

69. A method of treating cancer, comprising administering to a subject a TGFβ inhibitor in a therapeutically effective amount that does not induce a significant increase in platelet binding, activation, and/or aggregation.

70. The method of embodiment 69, wherein platelet binding, activation, and/or aggregation is measured in a sample of plasma or whole blood.

71. A method for determining whether a TGFβ inhibitor causes a significant increase in platelet binding, activation and/or aggregation following exposure of the sample to said TGFβ inhibitor, which method comprises measuring platelet binding, activation and/or aggregation in a blood sample.

72 The method of any one of embodiments 70 or 71, wherein the sample is obtained from the subject prior to administering a TGFβ inhibitor therapy.

73. The method of any one of embodiment 69-72, wherein the sample is obtained from a healthy subject.

74. The method of any one of embodiments 69-73, wherein administering the TGFβ inhibitor does not increase platelet binding by more than 10% as compared to binding in the absence of the TGFβ inhibitor and/or in the presence of a buffer or isotype control.

75. The method of any one of embodiments 69-74, wherein administering the TGFβ inhibitor does not increase platelet activation by more than 10%, as compared to activation in the absence of the inhibitor.

76. The method of any one of embodiments 69-75, wherein administering the TGFβ inhibitor does not increase platelet aggregation in vitro by more than 10% of the activation induced by a known platelet aggregation agonist, e.g., adenosine diphosphate (ADP).

77. The method of any one of embodiments 69-76, wherein administering the TGFβ inhibitor does not increase platelet aggregation by more than 10%, as compared to aggregation caused by a negative control.

78. The method of embodiments any one of 69-77, wherein the therapeutically effective amount of the TGFβ inhibitor is an amount sufficient to reduce levels of circulating MDSCs.

79. The method of embodiment 78, wherein the reduced MDSCs are G-MDSCs.

80. The method of embodiment 79, wherein the G-MDSCs express one or more of CD11 b, CD33, CD15, LOX-1, CD66b, and HLA-DR^{lo/-}.

81. The method of any one of embodiments 78-80, wherein the circulating MDSC levels are determined from whole blood or a blood component collected from the subject.

82. The method of any one of embodiments 69-81, wherein the TGFβ inhibitor is a TGFβ1 inhibitor, optionally a TGFβ1-specific inhibitor.

83. A TGFβ inhibitor for use in the treatment of cancer by administering to a subject a dose of said TGFβinhibitor, wherein said TGFβ inhibitor does not cause a significant increase in platelet binding, activation and/or aggregation.

84. A combination therapy comprising a dose of a TGFβ inhibitor and a cancer therapy agent for the treatment of cancer, wherein the treatment comprises simultaneous, separate, or sequential administration to a subject of a dose of the TGFβ inhibitor and the cancer therapy agent, wherein said TGFβ inhibitor does not cause a significant increase in platelet binding, activation and/or aggregation.

85. The TGFβ inhibitor for use according to embodiment 83 or the combination therapy for use according to embodiment 84, wherein the TGFβ inhibitor is administered in a therapeutically effective amount that is sufficient to reduce circulating MDSC levels.

86 The TGFβ inhibitor for use or the combination therapy for use according to embodiment 85, wherein the reduced MDSCs are G-MDSCs.

87 The TGFβ inhibitor for use or the combination therapy for use according to embodiment 86, wherein the G-MDSCs express one or more of CD11b, CD33, CD15, LOX-1, CD66b, and HLA-DR^{lo/-}.

88 The TGFβ inhibitor for use or the combination therapy for use according to embodiment 87, wherein the G-MDSCs express one or more of CD11b, CD33, CD15, LOX-1, CD66b, and HLA-DR^{lo/-}.

89. The method of embodiment 71 or any embodiment dependent thereon, the TGFβ inhibitor for use according to embodiment 83 or any embodiment dependent thereon, or the combination therapy for use according to embodiment 84 or any embodiment dependent thereon, wherein the TGFβ inhibitor is a TGFβ1 inhibitor, optionally a TGFβ1-specific inhibitor.

90. The TGFβ inhibitor for use according to embodiment 83 or any embodiment dependent thereon, or the combination therapy for use according to embodiment 84 or any embodiment dependent thereon, wherein the TGFβ inhibitor has been determined not to cause a significant increase in platelet binding, activation and/or aggregation by the method of embodiment 71 or any embodiment dependent thereon.

91. The TGFβ inhibitor for use according to embodiment 83 or any embodiment dependent thereon, or the combination therapy for use according to embodiment 84 or any embodiment dependent thereon, wherein the TGFβ inhibitor is a TGFβ inhibitor according to any one of embodiments 60-68.

92. A method of making a TGFβ inhibitor for treating cancer in a subject, comprising the steps of selecting a TGFβ inhibitor which satisfies one or more, e.g., all of, the following criteria:
a) the TGFβ inhibitor is efficacious in one or more preclinical models;
b) the TGFβ inhibitor does not cause valvulopathies or epithelial hyperplasia in toxicology studies in one or more animal species at a dose at least greater than a minimum efficacious dose; and
c) the TGFβ inhibitor does not induce significant cytokine release from human PBMCs or whole blood in an in vitro cytokine release assay at the minimum efficacious dose as determined in the one or more preclinical models of (a);

93 A method of making a TGFβ inhibitor for treating cancer in a subject, comprising the steps of selecting a TGFβ inhibitor which satisfies one or more, e.g., all of, the following criteria:
a) the TGFβ inhibitor is efficacious in one or more preclinical models;
b) the TGFβ inhibitor does not cause valvulopathies or epithelial hyperplasia in toxicology studies in one or more animal species at a dose at least greater than a minimum efficacious dose;
c) the TGFβ inhibitor does not induce significant cytokine release from human PBMCs or whole blood in an in vitro cytokine release assay at the minimum efficacious dose as determined in the one or more preclinical models of (a);
d) the TGFβ inhibitor does not induce a significant increase in platelet binding, activation, and/or aggregation at the minimum efficacious dose as determined in the one or more preclinical models of (a); and
e) the TGFβ inhibitor reduces circulating MDSCs at the minimum efficacious dose as determined in the one or more preclinical models of (a),
   wherein the method further comprises manufacturing a pharmaceutical composition comprising the TGFβinhibitor and a pharmaceutically acceptable excipient.

94. The method of embodiment 92 or 93, wherein the TGFβ inhibitor is a TGFβ1 inhibitor, optionally a TGFβ1-specific inhibitor.

95. A method of treating cancer in a subject, comprising administering a therapeutically effective amount of the TGFβ inhibitor manufactured according to the method of any one of embodiments 92-94.

96. A TGFβ inhibitor for use in an intermittent dosing regimen for cancer immunotherapy in a patient, wherein the intermittent dosing regimen comprises:
(i) measuring circulating MDSCs in a first sample, e.g., a blood sample, collected from the patient prior to a TGFβ inhibitor treatment,
(ii) administering a TGFβ inhibitor to the patient treated with a cancer therapy, wherein the cancer therapy is optionally a checkpoint inhibitor therapy,
(iii) measuring circulating MDSCs in a second sample collected from the patient after the TGFβ inhibitor treatment,
(iv) continuing with the cancer therapy if the second sample shows reduced levels of circulating MDSCs as compared to the first sample; and
(v) repeating the process as needed after a further blood sample from a patient shows elevated levels of circulating MDSC levels.

97 The method of embodiment 96, further comprising measuring circulating MDSCs in a third sample, and administering to the patient an additional dose of a TGFβ inhibitor if the third sample shows elevated levels of circulating MDSC levels as compared to the second sample.

98. The method of embodiment 96 or 97, wherein the TGFβ inhibitor inhibits TGFβ1 signaling.

99. The method of embodiment 96 or 97, wherein the TGFβ inhibitor inhibits TGFβ1 signaling but does not inhibit TGFβ2 signaling and/or TGFβ3 signaling at a therapeutically effective dose.

100. The method of embodiment 96 or 97, wherein the TGFβ inhibitor is TGFβ1-selective.

101. The method of embodiment 96 or 97, wherein the TGFβ inhibitor is an integrin inhibitor.

102. The method of 101, wherein the integrin inhibitor inhibits integrin αVβ1, αVβ3, αVβ5, αVβ6, αVβ8, α5β1, αIIbβ3, and/or α8β1.

103. The method of 101 or 102, wherein the integrin inhibitor inhibits downstream TGFβ1/3 activation.

104. A TGFβ1-selective inhibitor for use in the treatment of cancer in a subject, wherein the subject has been treated with a TGFβ inhibitor that inhibits TGFβ3 in conjunction with a checkpoint inhibitor.

105. The method of 104, wherein the cancer is a metastatic cancer, a desmoplastic tumor, or myelofibrosis.

106. The method of 104 or 105, wherein the subject has a disorder involving dysregulated extracellular matrix (ECM) or is at risk of developing such a disorder.

107. The method of 106, wherein the disorder involving dysregulated ECM is NASH,

108. The TGFβ1-selective inhibitor for use according to any one of embodiments 104-107, wherein the prior TGFβ inhibitor inhibits TGFβ1/2/3 or TGFβ1/3.

109. A non-isoform-selective TGFβ inhibitor for use in the treatment of cancer in a subject, comprising the steps of:
(i) selecting a subject who is not diagnosed with a fibrotic disorder or who is not at high risk of developing a fibrotic disorder; and,
(ii) administering to the subject the non-isoform-selective TGFβ inhibitor in an amount effective to treat the cancer.

110. An isoform-non-selective TGFβ inhibitor for use in the treatment of cancer in a subject, wherein the treatment comprises the steps of selecting a subject whose cancer is not a highly metastatic cancer and administering to the subject the isoform-non-selective TGFβ inhibitor.

111. The method of 109 or 110, wherein the isoform-non-selective TGFβ inhibitor is an antibody that inhibits TGFβ1/2/3 or TGFβ1/3.

112 The method of any one of embodiments 109-111, wherein the isoform-non-selective TGFβ inhibitor is an integrin inhibitor binding to integrins αVβ1, αVβ6, αVβ8, and/or αVβ3.

113 The method of 112, wherein the integrin inhibitor is an inhibitor of TGFβ1/3 activation.

114. The method of 110, wherein the isoform-non-selective TGFβ inhibitor is an engineered construct comprising a TGFβ receptor ligand-binding moiety.

115. The isoform-non-selective TGFβ inhibitor for use according to embodiment 110, wherein the highly metastatic cancer is colorectal cancer, lung cancer (e.g., NSCLC), bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, or thyroid cancer.

116. A TGFβ1-selective inhibitor for use in the treatment of cancer in a subject wherein the treatment comprises the steps of
(i) selecting a subject whose cancer is a highly metastatic cancer, and
(ii) administering to the subject an isoform-selective TGFβ1 inhibitor;
   wherein the highly metastatic cancer comprises colorectal cancer, lung cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, or thyroid cancer.

117. A TGFβ1-selective inhibitor for use in the treatment of cancer in a subject wherein the treatment comprises the steps of:
(i) selecting a subject having myelofibrosis, a fibrotic disorder or is at risk of developing a fibrotic disorder, and,
(ii) administering to the subject an isoform-selective TGFβ1 inhibitor in an amount effective to treat the cancer.

118. The TGFβ1-selective inhibitor for use according to embodiment 117, wherein the subject is further treated with a cancer therapy, wherein optionally the cancer therapy comprises a checkpoint inhibitor.

119. The method, the medical use, the TGFβ inhibitor for use, or the combination therapy for use according to any one of the preceding embodiments, wherein the subject is a patient who has not received cancer therapy.

120. The method, the medical use, the TGFβ inhibitor for use, or the combination therapy for use according to any one of the preceding embodiments, wherein the subject is receiving cancer therapy.

121. The method, the medical use, the TGFβ inhibitor for use, or the combination therapy for use according to any one of the preceding embodiments, wherein the subject has previously received cancer therapy.

122. The method, the medical use, the TGFβ inhibitor for use, or the combination therapy for use according to any one of the preceding embodiments, wherein the subject is or will be receiving cancer therapy.

123. The method, the medical use, the TGFβ inhibitor for use, or the combination therapy for use according to any one of the preceding embodiments, wherein the subject has cancer that is resistant to a cancer therapy that does not comprise a TGFβ inhibitor.

124. The method, the medical use, the TGFβ inhibitor for use, or the combination therapy for use according to any one of the preceding embodiments, wherein the subject is poorly responsive to a cancer therapy that does not comprise a TGFβ inhibitor.

125. The method, the medical use, the TGFβ inhibitor for use, or the combination therapy for use according to any one of the preceding embodiments, wherein the subject is currently receiving or previously received a cancer therapy that does not comprise a TGFβ inhibitor.

126. The method, the medical use, the TGFβ inhibitor for use, or the combination therapy for use according to any one of embodiments 121-125, wherein the cancer therapy does not comprise a TGFβ inhibitor.

127. The method, the medical use, the TGFβ inhibitor for use, or the combination therapy for use according to any one of embodiments 121-126, wherein the cancer therapy comprises a chemotherapy, radiation therapy (e.g., a radiotherapeutic agent), engineered immune cell therapy (e.g., CAR-T therapy), oncolytic viral therapy, and/or cancer vaccine therapy.

128. The method, the medical use, the TGFβ inhibitor for use, or the combination therapy for use according to any one of embodiments 121-127, wherein the cancer therapy comprises immunotherapy comprising a checkpoint inhibitor therapy.

129. A method of treating a subject having a solid cancer, comprising determining the level of cytotoxic T cells (e.g., CD8+ T cells) in a sample obtained from the subject prior to administering a TGFβ inhibitor, wherein the level of cytotoxic T cells (e.g., CD8+ T cells) inside the tumor is lower than the level of cytotoxic T cells (e.g., CD8+ T cells) outside the tumor prior to treatment, and administering to the subject a therapeutically effective amount of a TGFβ inhibitor, wherein the therapeutically effective amount is an amount sufficient to increase the level of cytotoxic T cells (e.g., CD8+ T cells) inside the tumor relative to the level outside the tumor.

130. A method of treating a subject having a solid cancer, comprising determining in a sample obtained from the subject the cytotoxic T cell (e.g., CD8+ T cell) levels inside and outside the tumor, selecting a subject having a ratio of cytotoxic T cell (e.g., CD8+ T cell) levels inside the tumor to outside the tumor of less than 1, and administering to the subject a therapeutically effective amount of a TGFβ inhibitor.

131. The method of embodiment 129 or 130, wherein the level of cytotoxic T cells (e.g., CD8+ T cells) outside the tumor is determined from the tumor margin and/or stroma.

132. The method of any one of embodiments 129-131, wherein the level of cytotoxic T cells (e.g., CD8+ T cells) outside of the tumor is determined from the margin.

133. The method of embodiment 131 or embodiment 132, wherein the margin is approximately 10-100 μm in width (e.g., 50 μm in width).

134. The method of any one of embodiments 129-133, wherein the level of the cytotoxic T cells (e.g., CD8+ T cells) in the margin and/or the stroma is at least 2-fold, 3-fold, 4-fold, 5-fold, 7-fold, or 10-fold greater than the level inside the tumor.

135. A method of treating a subject having a solid cancer, comprising measuring levels of CD8+ cells in one or more tumor nests from at least one tumor tissue sample obtained from the subject, and administering to the subject a therapeutically effective amount of a TGFβ inhibitor if greater than 50% of the area of the sample measured comprises tumor nests comprising lower levels of CD8-positive cells inside the tumor nest relative to levels of CD8-positive cells outside of the tumor nest (e.g., less than 5% CD8+ cells inside the tumor nest and greater than 5% CD8+ cells outside the tumor nest).

136. A method of treating a subject having a solid cancer, comprising:
(i) determining the cytotoxic T cell (e.g., CD8+ T cell) levels inside and outside the tumor in a first sample and selecting a subject having a ratio of cytotoxic T cell (e.g., CD8+ T cell) density inside the tumor to density outside the tumor of less than 1;
(ii) administering to the subject a first dose of a TGFβ inhibitor; and
(iii) determining the level of cytotoxic T cells (e.g., CD8+ T cells) inside the tumor in a second sample; and
(iv) administering to the subject a second dose of the TGFβ inhibitor if the level of cytotoxic T cells (e.g., CD8+ T cells) inside the tumor determined in step (iii) is increased as compared to the level of cytotoxic T cells (e.g., CD8+ T cells) inside the tumor determined in step (i).

137. A method of determining therapeutic efficacy of a cancer treatment in a subject comprising:
(i) determining the level of cytotoxic T cells (e.g., CD8+ T cells) inside the tumor in a first sample;
(ii) administering to the subject a dose of a TGFβ inhibitor;
(iii) determining the level of cytotoxic T cells (e.g., CD8+ T cells) inside the tumor in a second sample; and
(iv) determining whether the level of cytotoxic T cells (e.g., CD8+ T cells) determined in step (iii) is increased as compared to step (i), such increase being indicative of therapeutic efficacy of the cancer treatment.

138. The method of embodiment 136 or embodiment 137, wherein step (ii) further comprises administering to the subject an additional cancer therapy simultaneously, separately, or sequentially to the TGFβ inhibitor.

139. The method of embodiment 138, wherein the cancer therapy comprises a checkpoint inhibitor therapy (e.g., an agent targeting PD-1 or PD-L1, or an anti-PD-1 or anti-PD-L1 antibody).

140. The method of any one of embodiments 136-139, wherein the level of cytotoxic T cells (e.g., CD8+ T cells) in the tumor is increased by at least 10%, 15%, 20%, 25%, or more.

141. The method of any one of embodiments 129-140, wherein the TGFβ inhibitor is a TGFβ1-selective inhibitor, e.g., Ab6.

142. The method of any one of embodiments 129-141, wherein the sample is a tumor biopsy sample.

143. The method of embodiment 142, wherein the tumor biopsy sample is a core needle biopsy sample of the tumor.

144. The method of any of embodiments 129-143, wherein the level of cytotoxic T cells (e.g., CD8+ T cells) are determined by immunohistochemical analysis.

145. The method of embodiment 136, further comprising determining the level of circulating MDSCs before and after administration of the first dose of the TGFβ inhibitor, wherein a second dose of the TGFβ inhibitor is administered if a reduction of MDSC levels is determined after the administration of the first dose of the TGFβinhibitor and the level of cytotoxic T cells (e.g., CD8+ T cells) inside the tumor determined in step (iii) is increased as compared to the level of cytotoxic T cells (e.g., CD8+ T cells) inside the tumor determined in step (i).

146. The method of embodiment 137, further comprising determining the level of circulating MDSCs before and after administration of the TGFβ inhibitor, wherein a reduction of MDSC levels and/or an increase in cytotoxic T cells (e.g., CD8+ T cells) levels inside the tumor after the administration indicates therapeutic efficacy.

147 The method of embodiment 145 or 146, wherein the circulating MDSCs are G-MDSCs.

148. The method of embodiment 147, wherein the G-MDSCs express one or more of CD11b, CD33, CD15, LOX-1, CD66b, and HLA-DR^{lo/-}.

149. The method of any one of embodiments 145-148, wherein the circulating MDSC levels are determined from whole blood or a blood component collected from the subject.

150. The method of any one of embodiments 145-149, wherein the level of circulating MDSC levels is reduced by at least 10%, optionally by at least 15%, 20%, 25%, or more.

151. The method of any one of embodiments 145-150, wherein the level of cytotoxic T cells (e.g., CD8+ T cells) inside the tumor is increased by at least 10%, 15%, 20%, 25%, or more, and the level of circulating MDSCs is decreased by at least 15%, 20%, 25%, or more.

152. The method of any one of embodiments 129-151, wherein the level of cytotoxic T cells (e.g., CD8+ T cells) is the percentage of CD8+ T cells or the CD8+ cell density (e.g., number of CD8+ T cells per millimeter squared).

153. The method of any one of embodiments 129-152, wherein the therapeutically effective amount of the TGFβ inhibitor is between 0.1 mg/kg to 30 mg/kg per dose.

154. The method of any one of embodiments 129-153, wherein the therapeutically effective amount of the TGFβ inhibitor is between 1 mg/kg and 10 mg/kg per dose.

155. The method of any one of embodiments 129-154, wherein the therapeutically effective amount of the TGFβ inhibitor is between 2 mg/kg and 7 mg/kg per dose.

156. The method of any one of embodiments 129-155, wherein the TGFβ inhibitor is dosed weekly, every 2 weeks, every 3 weeks, every 4 weeks, monthly, every 6 weeks, every 8 weeks, bimonthly, every 10 weeks, every

12 weeks, every 3 months, every 4 months, every 6 months, every 8 months, every 10 months, or once a year.

157. The method of any one of embodiments 129-156, wherein the TGFβ inhibitor is dosed about every 3 weeks.

158. The method of any one of embodiments 129-157, wherein the TGFβ inhibitor is administered intravenously or subcutaneously.

159. The method of any one of embodiments 129-158, wherein the cancer is non-small cell lung cancer, melanoma, renal cell carcinoma, triple-negative breast cancer, gastric cancer, microsatellite stable-colorectal cancer, pancreatic cancer, small cell lung cancer, HER2-positive breast cancer, or prostate cancer.

160. A method of determining therapeutic efficacy of a cancer treatment in a subject, wherein the treatment comprises administering to the subject a combination therapy for simultaneous, separate or sequential administration comprising a dose of a TGFβ inhibitor and a cancer therapy, which method comprises:
(i) determining the circulating myeloid-derived suppressor cell (MDSC) level in a sample obtained from the subject prior to administering the TGFβ inhibitor, wherein optionally the MDSC level is determined by the use of an antibody that binds LRRC33;
(ii) determining the circulating MDSC level in a sample obtained from the subject after the administration of the TGFβ inhibitor, wherein optionally the MDSC level is determined by the use of an antibody that binds LRRC33; and
(iii) determining whether the level determined in step (ii) is reduced compared to the level determined in step (i), such reduction being indicative of therapeutic efficacy of the cancer treatment.

161. The method of embodiment 160, wherein the level of circulating MDSC cells is determined within 3-6 weeks following administration of the dose of TGFβ inhibitor, optionally within 3 weeks or at about 3 weeks following administration of the dose of TGFβ inhibitor.

162. The method of embodiment 160, wherein the level of circulating MDSC cells is determined within 2 weeks following administration of the dose of TGFβ inhibitor, optionally at about 10 days following administration of the dose of TGFβ inhibitor.

163. The method of any one of embodiments 160-162, wherein the subject in step (i) or (ii) has not received previous cancer therapy, optionally wherein the subject in steps (i) and (ii) has not received previous cancer therapy.

164. The method of any one of embodiments 160-163, wherein the subject is to receive the cancer therapy if circulating MDSC levels are determined to be reduced.

165. The method of any one of embodiments 160-164, wherein the subject in step (i) or (ii) has received previous cancer therapy or is receiving cancer therapy, optionally wherein the subject in step (i) and (ii) has received previous cancer therapy or is receiving cancer therapy.

166. The method of embodiment 165, wherein the subject is to receive further cancer therapy if circulating MDSC levels are determined to be reduced.

167. The method of any one of embodiments 160-166, wherein the subject receives more than one dose of the TGFβ inhibitor prior to step (ii).

168. The method of any one of embodiments 160-167, wherein the sample is a whole blood sample or a blood component.

169. A cancer therapy agent for use in the treatment of cancer in a subject, wherein the subject has received a dose of a TGFβ inhibitor and wherein the circulating MDSC level in the subject measured after the administration of the TGFβ inhibitor has been determined to be reduced as compared to the circulating MDSC level measured in the subject prior to administering the dose of the TGFβ inhibitor, wherein optionally the circulating MDSC level is measured using an antibody that binds LRRC33.

170. A combination therapy comprising a dose of a TGFβ inhibitor and a cancer therapy agent for use in the treatment of cancer, wherein the treatment comprises simultaneous, separate, or sequential administration to a subject of a dose of the TGFβ inhibitor and the cancer therapy agent, and wherein the circulating MDSC level in the subject measured after the administration of the TGFβ inhibitor has been determined to be reduced as compared to the circulating MDSC level measured in the subject prior to administering the dose of the TGFβinhibitor, wherein optionally the circulating MDSC level is measured using an antibody that binds LRRC33.

171. The combination therapy for use according to embodiment 170, wherein the subject has not received previous cancer therapy and wherein the circulating MDSC level in the subject has been determined to be reduced prior to administration of the cancer therapy agent.

172. The combination therapy for use according to embodiment 170 or embodiment 171, wherein the subject has not received previous cancer therapy, wherein the subject receives the TGFβ inhibitor prior to the cancer therapy agent.

173. The combination therapy for use according to embodiment 170, wherein the subject receives the cancer therapy agent prior to the TGFβ inhibitor.

174. A TGFβ inhibitor for use in the treatment of cancer in a subject, wherein the subject has received at least a first dose of the TGFβ inhibitor, and wherein the treatment comprises administering a further dose of the TGFβ inhibitor, provided that: the circulating MDSC level in the subject measured after the administration of the at least first dose of the TGFβ inhibitor is reduced as compared to the circulating MDSC level measured in the subject prior to administering a dose of the TGFβ inhibitor, wherein optionally the circulating MDSC level is measured using an antibody that binds LRRC33.

175. A TGFβ inhibitor for use in the treatment of cancer in a subject, wherein the subject is administered a dose of the TGFβ inhibitor, and wherein the TGFβ inhibitor reduces or reverses immune suppression in the cancer, wherein said reduced or reversed immune suppression has been determined by a reduction in the circulating MDSC level in the subject measured after the administration of the TGFβ inhibitor as compared to the circulating MDSC level measured in the subject prior to administering the dose of the TGFβ inhibitor, wherein optionally the circulating MDSC level is measured using an antibody that binds LRRC33.

176. The cancer therapy agent for use according to embodiment 169, the combination therapy for use according to embodiment 170 or any embodiment dependent thereon, or the TGFβ inhibitor for use according to embodiment 174 or embodiment 175, wherein the subject has received more than one dose of the TGFβ inhibitor prior to the determination that the circulating MDSC levels are reduced.

177. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the level of circulating MDSC cells is determined within 3-6 weeks following administration of the dose of TGFβ inhibitor, optionally within 3 weeks or at about 3 weeks, optionally within 2 weeks or at about 10 days, following administration of the dose of TGFβ inhibitor, optionally wherein said dose of TGFβ inhibitor is the first dose of the TGFβ inhibitor that the subject has received.

178. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has not received previous cancer therapy.

179. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject is receiving cancer therapy.

180. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject will be receiving cancer therapy.

181. The cancer therapy agent for use according to embodiment 169 or any embodiment dependent thereon, the combination therapy for use according to embodiment 170 or any embodiment dependent thereon, or the TGFβ inhibitor for use according to any one of embodiments 178-180, wherein the cancer therapy comprises immunotherapy, chemotherapy, radiation therapy, engineered immune cell therapy (e.g., CAR-T therapy), cancer vaccine therapy and/or oncolytic viral therapy.

182. The cancer therapy agent for use according to embodiment 169 or any embodiment dependent thereon, the combination therapy for use according to embodiment 170 or any embodiment dependent thereon, or the TGFβ inhibitor for use according to any one of embodiments 178-180, wherein the cancer therapy is immunotherapy comprising checkpoint inhibitor therapy, optionally wherein the checkpoint inhibitor comprises an agent targeting programmed cell death protein 1 (PD-1) or programmed cell death protein 1 ligand (PD-L1), optionally wherein the checkpoint inhibitor comprises an anti-PD-(L)1 antibody.

183. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the circulating MDSCs are G-MDSCs.

184. The method of determining therapeutic efficacy, the cancer therapy agent for use, the combination therapy for use, or TGFβ inhibitor for use according to embodiment 183, wherein the G-MDSCs express one or more of CD11b, CD33, CD15, LOX-1, CD66, and HLA-DR^{lo/-}.

185. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the circulating MDSC levels are reduced by at least 10%, optionally by at least 15%, 20%, 25%, or more.

186. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the circulating MDSC levels have been determined from a whole blood sample or a blood component obtained from the subject.

187. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the TGFβ inhibitor is a TGFβ1 inhibitor, optionally wherein the TGFβinhibitor is a TGFβ1-specific inhibitor.

188. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has circulating MDSC levels at least 2-fold above circulating MDSC levels in a healthy subject prior to a treatment.

189. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has or is suspected of having cancer.

190. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the level of a tumor-associated immune cell in the subject measured after the administration of the first dose of the TGFβ inhibitor is changed as compared to the level of said tumor-associated immune cell in the subject measured prior to the administration of the first dose of the TGFβ inhibitor.

191. The method of determining therapeutic efficacy according to embodiment 160 or any embodiment dependent thereon, which further comprises:
(iv) determining the level of one or more tumor-associated immune cells in a sample obtained from the subject prior to administering the TGFβ inhibitor;
(v) determining the level of one or more tumor-associated immune cells in a sample obtained from the subject after the administration of the TGFβ inhibitor; and
(vi) determining whether the level determined in step (v) is changed compared to the level determined in step (iv), such change being indicative of therapeutic efficacy of the cancer treatment.

192. The method according to embodiment 191, wherein a change in level of one or more tumor-associated immune cells indicates reduction or reversal of immune suppression in the cancer.

193. The method according to embodiment 191 or embodiment 192, wherein the tumor-associated immune cells comprise CD8+ T cells and/or tumor-associated macrophages (TAMs).

194. The method according to any one of embodiments 191-193, wherein the change in the levels of one or more tumor-associated immune cells comprises at least a 10%, optionally at least a 15%, 20%, 25%, or more, increase in CD8+ T cell levels.

195. The method according to any one of embodiments 191-194, wherein the change in the levels of one or more tumor-associated immune cells comprises at least a 10%, optionally at least a 15%, 20%, 25%, or more, increase in the level of TAMs.

196. The method according to any one of embodiments 191-195, wherein the level of one or more tumor-associated immune cells is determined, in a sample obtained from the subject, by immunohistochemistry analysis.

197. The method according to any one of embodiments 191-196, wherein the level of one or more tumor-associated immune cells is determined by in vivo imaging.

198. The method according to any one of embodiments 191-197, wherein the sample is a tumor biopsy sample, wherein the tumor biopsy sample is optionally a core needle biopsy sample of the tumor.

199. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the level of circulating latent TGFβ (e.g., circulating latent TGFβ1) in the subject measured after the administration of the first dose of the TGFβ inhibitor is changed as compared to the level of said circulating latent TGFβ in the subject measured prior to the administration of the first dose of the TGFβ inhibitor.

200. The method of determining therapeutic efficacy according to embodiment 160 or embodiment 191 or any embodiment dependent thereon, which further comprises:
(vii) determining the level of circulating latent TGFβ in a sample obtained from the subject prior to administering the TGFβ inhibitor;
(viii) determining the level of circulating latent TGFβ in a sample obtained from the subject after the administration of the TGFβ inhibitor; and
(ix) determining whether the level determined in step (viii) is increased compared to the level determined in step (vii), such increase being indicative of therapeutic efficacy of the cancer treatment.

201. The method according to embodiment 200, wherein the level of circulating latent TGFβ is determined in a sample obtained from the subject and wherein the sample is a whole blood sample or a blood component.

202. The method according to embodiment 200, wherein the circulating latent TGFβ is circulating latent TGFβ1.

203. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has cancer that is resistant to a cancer therapy that does not comprise a TGFβ inhibitor.

204. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject is poorly responsive to a cancer therapy that does not comprise a TGFβ inhibitor.

205. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject is currently receiving or previously received a cancer therapy that does not comprise a TGFβ inhibitor.

206. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has cancer that is resistant to a cancer therapy that does not comprise a TGFβ inhibitor.

207. The method, the medical use, the cancer therapy agent for use, the TGFβ inhibitor for use, or the combination therapy for use according to any one of the preceding embodiments, wherein the TGFβ inhibitor is administered to the subject intravenously.

208. The method, the medical use, the cancer therapy agent for use, the TGFβ inhibitor for use, or the combination therapy for use according to any one of the preceding embodiments, wherein the TGFβ inhibitor is administered at a concentration of about 37.5 mg/kg, 30 mg/kg, 20 mg/kg, 10 mg/kg, 7 mg/kg, 6 mg/kg, 5 mg/kg, 4 mg/kg, 3 mg/kg, 2 mg/kg, 1 mg/kg, or less.

209. The method, the medical use, the cancer therapy agent for use, the TGFβ inhibitor for use, or the combination therapy for use according to any one of the preceding embodiments, wherein the TGFβ inhibitor is administered in an amount of about 3000 mg, 2400 mg, 1600 mg, 800 mg, 240 mg, 80 mg, or less.

210. The method, the medical use, the cancer therapy agent for use, the TGFβ inhibitor for use, or the combination therapy for use according to embodiment 204 or embodiment 205, wherein the TGFβ inhibitor is administered about every three weeks.

211. The method, the medical use, the cancer therapy agent for use, the TGFβ inhibitor for use, or the combination therapy for use according to any one of the preceding embodiments, wherein the cancer comprises an immune-excluded tumor.

212. The method, the medical use, the cancer therapy agent for use, the TGFβ inhibitor for use, or the combination therapy for use according to any one of the preceding embodiments, wherein the cancer is a myeloproliferative disorder, wherein optionally the myeloproliferative disorder is myelofibrosis.

213. The method, the medical use, the cancer therapy agent for use, the TGFβ inhibitor for use, or the combination therapy for use according to any one of the preceding embodiments, wherein the cancer is a highly metastatic cancer.

214. The method, the medical use, the cancer therapy agent for use, the TGFβ inhibitor for use, or the combination therapy for use according to any one of the preceding embodiments, wherein the cancer is colorectal cancer, lung cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, or thyroid cancer.

215. The method, the medical use, the cancer therapy agent for use, the TGFβ inhibitor for use, or the combination therapy for use according to any one of the preceding embodiments, wherein the subject is at risk of developing aortic stenosis.

216. The method, the medical use, the cancer therapy agent for use, the TGFβ inhibitor for use, or the combination therapy for use according to any one of the preceding embodiments, wherein the cancer is TGFβ1-positive.

217. The method, the medical use, the cancer therapy agent for use, the TGFβ inhibitor for use, or the combination therapy for use according to any one of the preceding embodiments, wherein the cancer co-expresses TGFβ1 and TGFβ3.

218. The method, the medical use, the cancer therapy agent for use, the TGFβ inhibitor for use, or the combination therapy for use according to any one of the preceding embodiments, wherein the tumor is a TGFβ1-dominant tumor.

219. A method for manufacturing a pharmaceutical composition comprising a TGFβ inhibitor, the method comprising the steps of:
i) providing a TGFβ inhibitor that meets the following criteria:
   a) the TGFβ inhibitor is a monoclonal antibody, an antigen-binding fragment thereof, or a multispecific construct that is capable of binding a TGFβ;
   b) the TGFβ inhibitor binds the TGFβ with a K_{D} of <1.0 nM, preferably K_{D}<500 pM, as measured by a SPR-based assay (e.g., Biacore) and inhibits TGFβ1;
   c) the TGFβ inhibitor is effective in vivo in a preclinical model at a dose that does not cause a toxicity associated with pan-inhibition of TGFβ when dosed with at least 10 times the minimum efficacious amount for at least 4 weeks in an animal model;
ii) carrying out an immune safety assessment comprising:
   a) a cytokine release assay (in vitro and/or in vivo); and/or,
   b) a platelet assay
iii) producing the TGFβ inhibitor at a scale of 250 L or larger; and,
iv) formulating the TGFβ inhibitor into a pharmaceutical composition with one or more excipients.

220. The method of embodiment 219, wherein the TGFβ is TGFβ1.

221. The method of embodiment 219, wherein the TGFβ is a proTGFβ complex, mature TGFβ growth factor, or a ligand-binding domain of a TGFβ receptor.

222. The method of embodiment 219, wherein the TGFβ inhibitor is effective in causing tumor growth regression, prolonged survival, and/or normalized gene expression of PAI-1, CCL2, FN-1, ACTA2, Col1a1, Col3a1, FN-1, CTGF, and/or TGFβ1.

223. The method of embodiment 222, wherein the tumor is a TGFβ1-dominant tumor, wherein optionally the tumor further expresses TGFβ3.

224. The method of embodiment 219 wherein the toxicity associated with pan-inhibition of TGFβ comprises one or more of a cardiovascular toxicity (e.g., a valvulopathy), epithelial hyperplasia, bleeding, and skin lesion.

225. The method of embodiment 219, wherein the immune safety assessment comprises an in vitro cytokine release assay.

226. The method of embodiment 219, wherein the scale of the production is at least 500 L, at least 1000 L, at least 2000 L.

227. The method of any one of embodiments 219 to 226, wherein the production comprises a eukaryotic cell culture, wherein optionally the eukaryotic cell culture is a mammalian cell culture, plant cell culture, or an insect cell culture.

228. The method of embodiment 227, wherein the mammalian cell culture comprises a CHO cell, MDCK cell, NS0 cell, Sp2/0 cell, BHK cell, Murine C127 cell, Vero cell, HEK293 cell, HT-1080 cell, or PER.C6 cell.

229. A method of treating a TGFβ-related disorder in a subject, the method comprising administering to the subject a therapeutically effective amount of a TGFβ inhibitor to treat the disorder, wherein the therapeutically effective amount is an amount sufficient to increase the level of circulating latent TGFβ after the administration.

230. A method of treating a TGFβ-related disorder in a subject, the method comprising administering a TGFβinhibitor and monitoring levels of circulating latent TGFβ after administration.

231. The method of embodiments 229 or 230, wherein the TGFβ-related disorder is a TGFβ1-related disorder.

232. The method of embodiment 231, wherein the TGFβ1-related disorder is a cancer.

233. The method of embodiment 231, wherein the TGFβ1-related disorder is an immune disorder

234. The method of any one of embodiments 229-233, wherein if the level of circulating latent TGFβ after the administration of the TGFβ inhibitor is increased, e.g., by at least 2-fold, 3-fold, 4-fold, 5-fold, 6-fold, 7-fold, 8-fold, 9-fold, 10-fold, or more, relative to the level prior to the administration, an additional dose of the TGFβinhibitor is administered.

235. The method of any one of embodiments 229-234, wherein the level of circulating latent TGFβ after the administration of the TGFβ inhibitor is increased to a maximum of at least 1000 μg/ml.

236. The method of any one of embodiments 229-235, wherein the level of circulating latent TGFβ after the administration of the TGFβ inhibitor is increased to a maximum of about 1000 μg/ml to about 8000 μg/ml.

237. The method of any one of embodiments 229-236, wherein the level of circulating latent TGFβ after the administration of the TGFβ inhibitor is increased to a maximum about 2000 μg/ml to about 6500 μg/ml.

238. The method of any one of 229-237, wherein the level of circulating latent TGFβ after the administration of the TGFβ inhibitor is increased by a minimum of about 1.5-fold.

239. The method of any one of embodiments 229-238, wherein the level of circulating latent TGFβ is measured about 8 to about 672 hours following administration of the TGFβ inhibitor.

240. The method of any one of embodiments 229-239, wherein the level of circulating latent TGFβ is measured about 24 hours to about 336 hours following administration of the TGFβ inhibitor.

241. The method of any one of embodiments 229-240, wherein the level of circulating latent TGFβ is measured about 72 hours to about 240 hours following administration of the TGFβ inhibitor.

242. The method of any one of embodiments 229-241, wherein the TGFβ inhibitor is administered at a dose of about 1 mg/kg to about 30 mg/kg.

243. The method of any one of embodiments 229-242, wherein the TGFβ inhibitor is administered at a dose of about 5 mg/kg to about 20 mg/kg.

244. The method of any one of embodiments 229-243, wherein the TGFβ inhibitor is administered at a dose of about 2 mg/kg to about 7 mg/kg.

245. The method of any one of embodiments 229-245, wherein the TGFβ inhibitor is administered about every three weeks.

246. A method of determining the efficacy of a cancer treatment in a subject, comprising determining the level of circulating latent TGFβ1 in a first sample from the subject, administering a dose of a TGFβ1 inhibitor to the subject, and determining the level of circulating latent TGFβ in a second sample from the subject after administration, wherein an increase of at least 2-fold, 3-fold, 4-fold, 5-fold, 6-fold, 7-fold, 8-fold, 9-fold, 10-fold, or more in circulating latent TGFβ levels between the first sample and the second sample indicates treatment efficacy.

247. A method of treating a subject with a solid cancer, comprising determining the level of circulating latent TGFβ1 in a first sample from the subject, administering to the subject a dose of a TGFβ1 inhibitor, and determining the level of circulating latent TGFβ in a second sample from the subject after administration.

248. The method of embodiment 247, wherein if the subject has a ratio of circulating latent TGFβ after the administration to before the administration of at least 1.2, an additional dose of the TGFβ inhibitor is administered.

249. The method of any one of embodiments 246-248, wherein the second sample is collected from the subject 24 hours to 56 days after the administration.

250. The method of any one of embodiments 229-249, wherein the TGFβ inhibitor is a TGFβ activation inhibitor, e.g., a TGFβ1-selective inhibitor.

251. The method of embodiment 2250, wherein the TGFβ inhibitor is Ab6.

252. The method of any one of embodiments 229-251, wherein the therapeutically effective amount of the TGFβ inhibitor is between 0.1 mg/kg to 30 mg/kg per dose.

253. The method of any one of embodiments 229-252, wherein the therapeutically effective amount of the TGFβ inhibitor is between 1 mg/kg and 10 mg/kg per dose.

254. The method of any one of embodiments 229-253, wherein the therapeutically effective amount of the TGFβ inhibitor is between 2 mg/kg and 7 mg/kg per dose.

255. The method of any one of embodiments 229-254, wherein the TGFβ inhibitor is dosed weekly, every 2 weeks, every 3 weeks, every 4 weeks, monthly, every 6 weeks, every 8 weeks, bimonthly, every 10 weeks, every

12 weeks, every 3 months, every 4 months, every 6 months, every 8 months, every 10 months, or once a year.

256. The method of any one of embodiments 229-255, wherein the TGFβ inhibitor is dosed about every 3 weeks.

257. The method of any one of embodiments 229-256, wherein the TGFβ inhibitor is administered intravenously or subcutaneously.

258. The method of any one of embodiments 229-257, wherein the latent TGFβ is latent TGFβ1.

259. The method of any one of embodiments 229-258, wherein the level of circulating latent TGFβ is measured in a blood sample.

260. The method of any one of embodiments 229-259, wherein the blood sample is a serum sample or a plasma sample.

261. The method of any one of embodiments 229-260, wherein the circulating latent TGFβ levels are measured by ELISA.

262. The method of any one of embodiments 229-261, further comprising determining the levels of circulating MDSCs in the subject prior to and after administration of the TGFβ inhibitor, wherein optionally the circulating MDSC levels are determined with the use of an antibody that binds LRRC33.

263. The method of embodiment 262, wherein a reduction in the levels of circulating MDSCs after the administration as compared to before the administration indicates therapeutic efficacy and, optionally, one or more additional treatments comprising the TGFβ inhibitor is administered.

264. The method of embodiment 262, wherein the circulating MDSCs are G-MDSCs.

265. The method of embodiment 264, wherein the G-MDSCs express one or more of CD11 b, CD33, CD15, LOX-1, CD66b, and HLA-DR^{lo/-}.

266. The method of any one of embodiments 243-246, wherein the circulating MDSC levels are determined from whole blood or a blood component collected from the subject.

267. The method of any one of embodiments 262-266, wherein administration of the TGFβ inhibitor reduces circulating MDSC levels by at least 10%, optionally by at least 15%, 20%, 25%, or more.

268. The method of any one of embodiments 262-267, wherein circulating latent TGFβ levels are increased by at least 50% and circulating MDSC levels are decreased by at least 15%, 20%, 25%, or more.

269. The method, the medical use, the cancer therapy agent for use, the TGFβ inhibitor for use, or the combination therapy for use according to any one of the preceding embodiments, wherein the TGFβ inhibitor inhibits TGFβ1 signaling.

270. The method, the medical use, the cancer therapy agent for use, the TGFβ inhibitor for use, or the combination therapy for use according to any one of the preceding embodiments, wherein the TGFβ inhibitor Inhibits TGFβ1 signaling but does not inhibit TGFβ3 signaling at a therapeutically effective dose.

271. The method, the medical use, the cancer therapy agent for use, the TGFβ inhibitor for use, or the combination therapy for use according to any one of the preceding embodiments, wherein the TGFβ inhibitor does not inhibit TGFβ2 signaling and TGFβ3 signaling at a therapeutically effective dose.

272. The method, the medical use, the cancer therapy agent for use, the TGFβ inhibitor for use, or the combination therapy for use according to any one of the preceding embodiments, wherein the TGFβ inhibitor does not bind to free TGFβ growth hormones.

273. The method, the medical use, the cancer therapy agent for use, the TGFβ inhibitor for use, or the combination therapy for use according to any one of the preceding embodiments, wherein the TGFβ inhibitor binds to pro- and/or -latent TGFβ1.

274. The method, the medical use, the cancer therapy agent for use, the TGFβ inhibitor for use, or the combination therapy for use according to any one of the preceding embodiments, wherein the TGFβ inhibitor binds to at least a portion of a Latency Lasso in TGFβ1.

275. The method, the medical use, the cancer therapy agent for use, the TGFβ inhibitor for use, or the combination therapy for use according to any one of the preceding embodiments, wherein the TGFβ inhibitor binds to at least a portion of a Finger-1 domain in TGFβ1.

276. The method, the medical use, the cancer therapy agent for use, the TGFβ inhibitor for use, or the combination therapy for use according to any one of the preceding embodiments, wherein the TGFβ inhibitor is a neutralizing antibody or a ligand trap.

277. The method, the medical use, the cancer therapy agent for use, the TGFβ inhibitor for use, or the combination therapy for use according to any one of the preceding embodiments, wherein the TGFβ inhibitor binds selectively to TGFβ1, optionally selectively to a pro- and/or latent-TGFβ1.

278. The method, the medical use, the cancer therapy agent for use, the TGFβ inhibitor for use, or the combination therapy for use according to any one of the preceding embodiments, wherein the TGFβ inhibitor is an isolated antibody or antigen-binding fragment thereof which is capable of specifically binding a proTGFβ1 complex at (i) a first binding region comprising at least a portion of Latency Lasso (SEQ ID NO: 1126); and ii) a second binding region comprising at least a portion of Finger-1 (SEQ ID NO: 1124).

279. The method, the medical use, the cancer therapy agent for use, the TGFβ inhibitor for use, or the combination therapy for use according to embodiment 278, wherein the first binding region further comprises an amino acid sequence of SEQ ID NO: 1134 or a portion thereof.

280. The method, the medical use, the cancer therapy agent for use, the TGFβ inhibitor for use, or the combination therapy for use according to embodiment 278, wherein the second binding region further comprises an amino acid sequence of SEQ ID NO: 1143 or a portion thereof.

281. The method, the medical use, the cancer therapy agent for use, the TGFβ inhibitor for use, or the combination therapy for use according to any one of the preceding embodiments, wherein the TGFβ inhibitor comprises an isolated antibody or antigen-binding fragment thereof, comprising three heavy chain complementarity determining regions comprising amino acid sequences of SEQ ID NO: 1001 (H-CDR1), SEQ ID NO: 1002 (H-CDR2), and SEQ ID NO: 1003 (H-CDR3), and three light chain complementarity determining regions comprising amino acid sequences of SEQ ID NO: 1004 (L-CDR1), SEQ ID NO: 1005 (L-CDR2), and SEQ ID NO: 1006 (L-CDR3), as defined by the IMTG numbering system.

282. The method, the medical use, the cancer therapy agent for use, the TGFβ inhibitor for use, or the combination therapy for use according to any one of the preceding embodiments, wherein the TGFβ inhibitor comprises an isolated antibody or antigen-binding fragment thereof, comprising three heavy chain complementarity determining regions (H-CDR1, H-CDR2, and H-CDR3) from a heavy chain variable region comprising an amino acid sequence of SEQ ID NO: 1007, and three light chain complementarity determining regions (L-CDR1, L-CDR2, and L-CDR3) from a light chain variable region comprising an amino acid sequence of SEQ ID NO: 1008.

283. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising three heavy chain complementarity determining regions (H-CDR1, H-CDR2, and H-CDR3) from a heavy chain variable region that is at least 90% identical to an amino acid sequence of SEQ ID NO: 1007, and three light chain complementarity determining regions (L-CDR1, L-CDR2, and L-CDR3) from a light chain variable region that is at least 90% identical to an amino acid sequence of SEQ ID NO: 1008.

284. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising a heavy chain variable region that is at least 90% identical to an amino acid sequence of SEQ ID NO: 1007 and a light chain variable region that is at least 90% identical to an amino acid sequence of SEQ ID NO: 1008.

285. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising a heavy chain variable region comprising an amino acid sequence of SEQ ID NO: 1007 and a light chain variable region comprising an amino acid sequence of SEQ ID NO: 1008.

286. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising a heavy chain comprising an amino acid sequence of SEQ ID NO: 1009 and a light chain comprising an amino acid sequence of SEQ ID NO: 1011.

287. The method, the medical use, the cancer therapy agent for use, the TGFβ inhibitor for use, or the combination therapy for use according to any of the preceding embodiments, wherein the TGFβ inhibitor cross-blocks and/or competes for binding to TGFβ1 with an antibody or antigen-binding fragment comprising a heavy chain variable domain of SEQ ID NO: 1007, and a light chain variable domain of SEQ ID NO: 1008.

288. The method, the medical use, the cancer therapy agent for use, the TGFβ inhibitor for use, or the combination therapy for use according to any one of the preceding embodiments, wherein the TGFβ inhibitor is a monoclonal antibody, optionally a fully human or humanized antibody, or an antigen binding fragment thereof.

289. The method, the medical use, the cancer therapy agent for use, the TGFβ inhibitor for use, or the combination therapy for use according to any one of the preceding embodiments, wherein the TGFβ inhibitor is present in a multispecific or bispecific construct.

290. The method, the medical use, the cancer therapy agent for use, the TGFβ inhibitor for use, or the combination therapy for use according to embodiment 289, wherein the multispecific or bispecific construct is also capable of binding to an immune cell-surface antigen,

291. The method, the medical use, the cancer therapy agent for use, the TGFβ inhibitor for use, or the combination therapy for use according to embodiment 290, wherein the immune cell-surface antigen is PD-1, PD-L1, CTLA4, or LAG3.

292. The method, the medical use, the cancer therapy agent for use, the TGFβ inhibitor for use, or the combination therapy for use according to embodiment 290 or 291, wherein the immune cell-surface antigen is PD-1 or PD-L1, optionally comprising an anti-PD-1 or anti-PD-L1 antibody or antigen binding fragment thereof.

293. The method, the medical use, the cancer therapy agent for use, the TGFβ inhibitor for use, or the combination therapy for use according to any of the preceding embodiments, wherein the TGFβ inhibitor comprises a human IgG₄ or IgG₁ constant region.

294. The method, the medical use, the cancer therapy agent for use, the TGFβ inhibitor for use, or the combination therapy for use according to any of the preceding embodiments, wherein the subject is a human patient and wherein the patient has a historically documented solid tumor that is metastatic or locally advanced, for which standard-of-care therapy does not exist, has failed in the patient, or is not tolerated by the patient, or for which the patient has been assessed as not suitable candidate or otherwise ineligible for the standard-of-care therapy.

295. The method, the medical use, the cancer therapy agent for use, the TGFβ inhibitor for use, or the combination therapy for use according to any of the preceding embodiments, wherein the subject is a human patient and wherein the patient has a history of primary anti-PD-(L)1 antibody nonresponse presenting either as progressive disease or stable disease (e.g., not improving, but also not worsening, clinically or radiographically) after at least 3 cycles of treatment with an anti-PD-(L)1 antibody therapy (optionally alone or in combination with chemotherapy) approved for that tumor type.

296. The method, the medical use, the cancer therapy agent for use, the TGFβ inhibitor for use, or the combination therapy for use according to any of the preceding embodiments, wherein the subject is a human patient and wherein the patient has received the most recent dose of anti-PD-(L)1 antibody therapy within 6 months of the administration of the TGFβ inhibitor.

297. The method, the medical use, the cancer therapy agent for use, the TGFβ inhibitor for use, or the combination therapy for use according to any of the preceding embodiments, wherein the subject is a human patient and wherein the patient has NSCLC and has genomic tumor aberrations for which a targeted therapy is available (wherein optionally the targeted therapy targets anaplastic lymphoma kinase and/or EGFR), wherein further optionally the patient has progressed on an approved therapy for these aberrations or did not tolerate an approved therapy for these aberrations, or was not considered suitable candidates or was otherwise ineligible for an approved therapy for these aberrations.

298. The method, the medical use, the cancer therapy agent for use, the TGFβ inhibitor for use, or the combination therapy for use according to any of the preceding embodiments, wherein the subject is a human patient and wherein the patient has measurable disease as determined by Response Evaluation Criteria in Solid Tumor (RECIST) v1.1.

299. The method, the medical use, the cancer therapy agent for use, the TGFβ inhibitor for use, or the combination therapy for use according to any of the preceding embodiments, wherein the subject is a human patient and wherein the patient has an Eastern Cooperative Oncology Group performance status (PS) 0-1.

300. The method, the medical use, the cancer therapy agent for use, the TGFβ inhibitor for use, or the combination therapy for use according to any of the preceding embodiments, wherein the subject is a human patient and wherein the patient has a predicted life expectancy of ≥3 months.

301. The method of embodiment 1, wherein the reduced circulating MDSCs are M-MDSCs.

302. The method of embodiment 1 or embodiment 2, wherein the M-MDSCs express one or more of CD11b+ CD33+ CD14+ CD15− and HLA-DR^{-/lo}.

303. The composition, composition for use, or method of any one of the preceding embodiments, wherein the TGFβ inhibitor is shown to cause no significant adverse events (e.g., dose-limiting toxicities) in a preclinical animal model when dosed at up to 100, 200, or 300 μg/kg weekly for 4 weeks, 8 weeks or up to 12 weeks, as assessed by standard toxicology analyses or according to the present disclosure.

304. The composition for use, the TGFβ inhibitor for use, or the method according to any one of the preceding embodiments, wherein selection of the composition or the TGFβ inhibitor comprises in vivo efficacy and safety criteria, wherein the safety criteria includes: i) lack of platelet aggregation, activation and/or binding when assessed under the condition according to the present disclosure, and, ii) lack of significant (e.g., within 2.5-fold of control) cytokine release, when assessed under the condition according to the present disclosure.

305. The composition for use, the TGFβ inhibitor for use, or the method according to any one of the preceding embodiments, wherein the pharmacodynamics of the TGFβ inhibitor are assessed by measuring circulatory latent TGFβ1 levels before and after the administration of the TGFβ1 inhibitor in blood (serum) samples collected from the subject.

306. A TGFβ inhibitor for use in the treatment of cancer comprising a solid tumor in a patient, wherein the treatment comprises administration of the TGFβ inhibitor in conjunction with a checkpoint inhibitor (CPI) to treat the cancer, wherein the solid tumor has an immune-infiltrated phenotype and is resistant or refractory to a CPI therapy.

307. The TGFβ1 inhibitor for use according to embodiment 306, wherein the TGFβ inhibitor is a TGFβ1 inhibitor.

308. The TGFβ inhibitor for use according to embodiment 307, wherein the TGFβ1 inhibitor is a TGFβ1-selective inhibitor.

309. The TGFβ inhibitor for use according to any one of embodiments 306 to 308, wherein the TGFβ inhibitor is capable of binding to a GARP-TGFβ1 complex.

310. The TGFβ inhibitor for use according to any one of embodiments 306 to 309, wherein the TGFβ inhibitor is capable of binding to a GARP-TGFβ1 complex and a LRRC33-TGFβ1 complex.

311. The TGFβ inhibitor for use according to any one of embodiments 306 to 310, wherein the TGFβ inhibitor is capable of binding to a GARP-TGFβ1 complex, a LRRC33-TGFβ1 complex, a LTBP1-TGFβ1 complex, and a LTBP3-TGFβ1 complex.

312. The TGFβ inhibitor for use according to any one of embodiments 309 to 311, wherein the TGFβinhibitor inhibits the release of mature TGFβ1 from the pro/latent complex.

313. The TGFβ inhibitor for use according to any one of embodiments 306 to 312, wherein the TGFβ inhibitor inhibits integrin-dependent activation of TGFβ1.

314. The TGFβ inhibitor for use according to any one of embodiments 306 to 313, wherein the TGFβ inhibitor is an antibody or antigen-binding fragment thereof.

315. The TGFβ inhibitor for use according to embodiment 314, wherein the antibody or antigen-binding fragment thereof comprises the CDRs of any one of Ab4, Ab5, Ab6, Ab21, Ab22, Ab23, Ab24, Ab25, Ab26, Ab27, Ab28, Ab29, Ab30, Ab31, Ab32, Ab33, and Ab34, or Ab46.

316. The TGFβ inhibitor for use according to embodiment 314, wherein the antibody or antigen-binding fragment thereof comprises the heavy and light chain variable regions of any one of Ab4, Ab5, Ab6, Ab21, Ab22, Ab23, Ab24, Ab25, Ab26, Ab27, Ab28, Ab29, Ab30, Ab31, Ab32, Ab33, and Ab34, or Ab46.

317. The TGFβ inhibitor for use according to embodiment 314, wherein the antibody is any one of Ab4, Ab5, Ab6, Ab21, Ab22, Ab23, Ab24, Ab25, Ab26, Ab27, Ab28, Ab29, Ab30, Ab31, Ab32, Ab33, and Ab34, or Ab46.

318. The TGFβ inhibitor for use according to embodiment 314, wherein the antibody is Ab6.

319. The TGFβ inhibitor for use according to any one of embodiments 306 to 318, wherein the solid tumor is a carcinoma.

320. The TGFβ inhibitor for use according to embodiment 319, wherein the carcinoma comprises cells that have undergone epithelial-to-mesenchymal transition (EMT).

321. The TGFβ inhibitor for use according to any one of embodiments 319 or 320, wherein the carcinoma is renal cell carcinoma (RCC).

322. The TGFβ inhibitor for use according to embodiment 321, wherein the renal cell carcinoma is clear cell renal cell carcinoma (ccRCC).

323. The TGFβ inhibitor for use according to any one of embodiments 306 to 322, wherein the cancer comprises infiltrated CD8+ cells that have reduced cytotoxic function, optionally wherein the CD8+ cells express reduced amounts of cytotoxic enzymes, such as perforin and/or granzyme B.

324. The TGFβ inhibitor for use according to any one of embodiments 306 to 323, wherein the CPI is a PD-1 antagonist, a PD-L1 antagonist, or a CTLA4 antagonist.

It will be readily apparent to those skilled in the art that other suitable modifications and adaptations of the composition and methods described herein may be made using suitable equivalents without departing from the scope of the disclosure or the embodiments disclosed herein. This disclosure is further illustrated by the following examples which should not be construed as limiting.

### EXAMPLES

### Example 1: Measurement of Latent TGFβ Activation

Inhibitory activity of Ab6 was measured as previously described in Martin et al., 2020. Briefly, LN229 cells (ATCC) were transfected with a plasmid encoding either human, rat, or cynomolgus macaque proTGFβ1. About 24 hours after cell transfection, Ab6 was added to the transfectants together with CAGA12 reporter cells (Promega, Madison, WI). Approximately 16-20 hours after setting up the co-culture, the assay was developed and luminescence read out on a plate reader. Dose-response activities were nonlinearly fit to a three-parameter log inhibitor vs. response model using Prism 8 and best-fit IC50 values calculated.

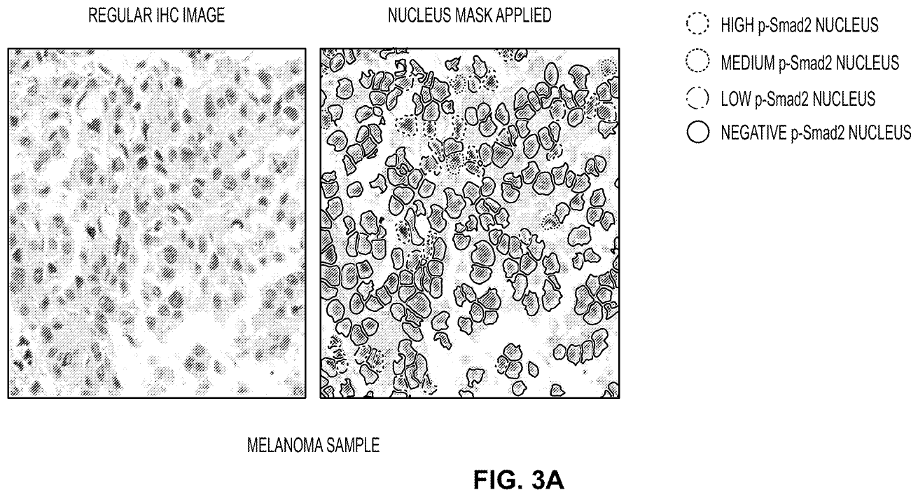

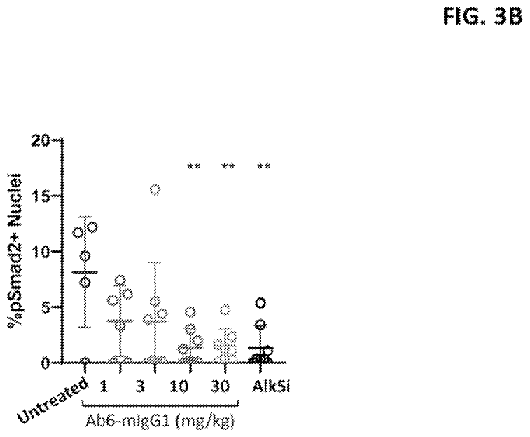

Modulation of TGFβ signaling in Ab6-treated tumors will be detected using a P-Smad 2 IHC assay, where decreased expression in P-Smad2 is indicative of TGFβ inhibition. As shown in FIG. 3A, a P-Smad2 IHC assay was developed using commercially available melanoma samples and a digital nuclear masking analysis. Nucleus mask is a digital image analysis parameter that enables visualization and measurement of IHC signal intensity of an individually marked cell or nucleus. The assay was established using the commercial melanoma samples to enable identification of a range of P-Smad2 nucleus staining intensity ranging from high (red), medium (orange), low (yellow) to negative (blue). Cells were sorted into categories (e.g., 0, 1+, 2+, 3+) based on chromogenic intensity relative to scanning parameters. Variables analyzed during assay development are shown in Table 13 below. Analysis was carried out using software developed by Flagship Biosciences.

TABLE 13
Primary Data Variables for P-Smad 2 IHC Assay

| Variable Name | Description |
|---|---|
| Total Number of Quantified Cells | Total number of cells quantified by IA in compartment |
| Percent Total Cells | Percent of Cells in compartment out of All Cells |
| Number of p-Smad2 positive Cells | Number of p-Smad2 positive cells in compartment |
| Number of p-Smad2 negative Cells | Number of p-Smad2 negative cells in compartment |
| Percent Positive Cells | Percent of p-Smad2 in the compartment identified |
| Compartment Area (mm²) | Area of compartment in mm² |
| Compartment Area (Percent) | Percent of the total tissue area occupied by compartmental area |
| % 0 p-Smad2 cells | Percent of cells at 0 intensity |
| % 1+ p-Smad2 cells | Percent of cells at 1+ intensity |
| % 2+ p-Smad2 cells | Percent of cells at 2+ intensity |
| % 3+ p-Smad2 cells | Percent of cells at 3+ intensity |
| Number of 0 p-Smad2 cells | Number of cells at 0 intensity |
| Number of 1+ p-Smad2 cells | Number of cells at 1+ intensity |
| Number of 2+ p-Smad2 cells | Number of cells at 2+ intensity |
| Number of 3+ p-Smad2 cells | Number of cells at 3+ intensity |
| H-Score | Standard scoring paradigm representing both the prevalence and |
| | intensity of biomarker expression. H-Score scale ranges from 0-300. |

pSmad2 phosphorylation was measured in MBT2 mice, which further supported the rationale for measuring tumor phosphorylated smad 2 (p-Smad2) as a biomarker for SRK-181-mediated TGFβ1 inhibition. As shown in FIG. 3B, p-Smad2 levels were analyzed in MBT-2 mice (bladder cancer) at day 10 following weekly dosing of Ab6-mIgG1 at 1 mg/kg, 3 mg/kg, 10 mg/kg, or 30 mg/kg. Ab6-mIgG1 treatment reduced tumor p-Smad2 phosphorylation, thereby supporting p-Smad2 as a pharmacodynamic biomarker.

Briefly, samples from MBT-2 mice were fixed, processed, embedded, and sectioned at 4 μm thickness. Slides were deparaffinized and stained using a Leica Bond Rx autostainer. Sections were blocked using a 1× diluted Animal Free Blocking solution from Cell Signaling Technologies (#15019). Staining was performed using a phospho-Smad2 rabbit monoclonal antibody from Cell Signaling Technologies (#3108, 138D4) at a final concentration of 0.174 ug/mL. Heat induced epitope retrieval was performed using a citrate-based solution for 20 minutes at 95° C. Polymer-Rabbit HRP was incubated as a post-primary for 25 minutes, procured from Cell Signaling Technologies (#8114S). Secondary fluorophores, spectral DAPI, and Tyramide Signal Amplification reagents were procured from Akoya Biosciences (NEL741001KT, FP1490). Sections were dried and cover-slipped with antifade mounting media (ThermoFisher, P36961), and imaged on a Zeiss AxioScan z1 with an additional Cy5 band to further separate autofluorescence signal.

Image quantification was performed using a Visiopharm analysis platform. Nuclei were segmented first on the basis of DAPI staining intensity and resulting objects were reclassified as pSmad2 strong, medium, or weakly positive. These classifications were thresholded by FITC intensity, with a background threshold of 15000 ABU and requiring at least 30% nuclear coverage. Segmented objects with area less than 23 um2 or greater than 230 um2 were discarded before pSmad2 classifications were applied. Strong, medium, and weak classifications were used for QC purposes, and total % pSmad2 positivity is reported as the sum of these groups, divided by the total number of cells, and multiplied by 100.

### Example 2: In Vivo Assessment of LRRC33 Expression on Circulating MDSC in MBT2 Syngeneic Bladder Cancer Mouse Model

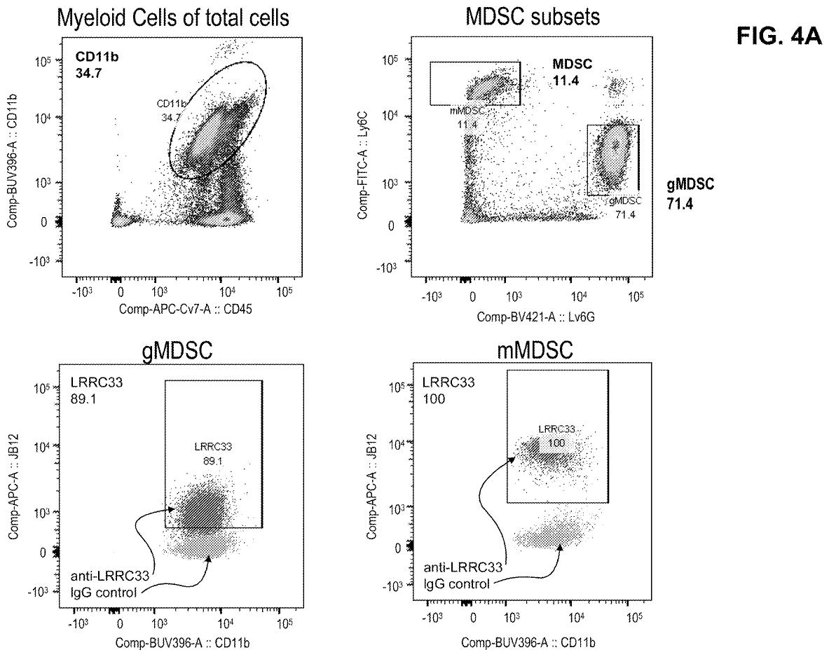

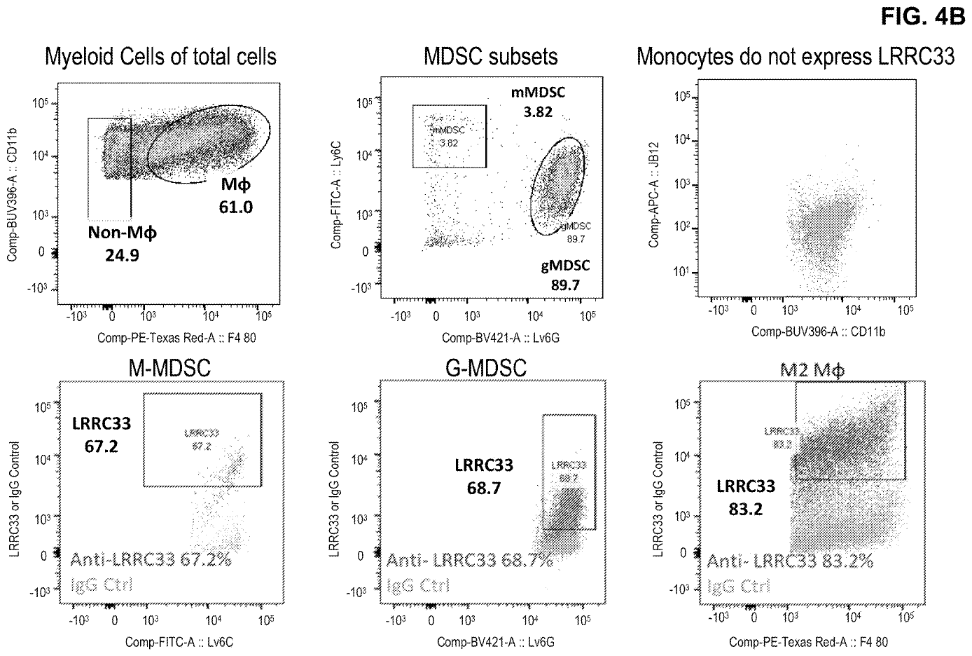

LRRC33 expression levels on circulating MDSCs were determined using FACS analysis using whole blood samples collected from mice bearing MBT2 tumors. M-MDSC and G-MDSC populations were identified on the basis of Ly6C and Ly6G expression, respectively, and then further characterized using a monoclonal antibody that binds cell-surface LRRC33, and an IgG control. Nearly all circulating MDSCs, including both G-MDSCs and M-MDSCs, appeared to express LRRC33. As shown in FIG. 4A, LRRC33 expression was detected on 89.1% of G-MDSCs and 100% of the M-MDSCs from whole blood of the tumor bearing mice. Consistent with previous observations, LRRC33 was also detected on intratumoral MDSCs, including G-MDSCs and M-MDSCs, albeit at varying levels (FIG. 4B). In contrast, LRRC33 expression was not detected on monocytes.

### Example 3: Phase 1 Clinical Study

A multi-center, open-label, Phase 1, first-in-human, dose escalation and dose expansion study (DRAGON; NCT04291079) is ongoing to evaluate the safety, tolerability, pharmacokinetics, pharmacodynamics, and efficacy of SRK-181 (a fully human, monoclonal antibody of IgG4 subtype, which specifically binds to the LLCs of TGFβ1, which is also referred to as Ab6 herein) administered alone and in combination with anti-PD-(L)1 therapy in adult patients with locally advanced or metastatic solid tumors. The study is divided into three treatment parts, Part A1, Part A2, and Part B, and a Long-Term Extension Phase (LTEP). Part A1 (dose escalation) determines the maximum tolerated dose (MTD) or maximum administered dose (MAD) of SRK-181 as a single agent and the recommended Phase 2 dose (RP2D) of SRK-181 as a single-agent. Part A2 (dose escalation) determines the MTD or MAD of SRK-181 in combination with anti-PD-(L)1 antibody therapy and the RP2D of SRK-181 in combination with anti-PD-(L)1 antibody therapy for use in Part B. In Part B (dose expansion), parallel cohorts of patients with Non-small cell lung cancer (NSCLC), Urothelial Carcinoma (UC), Cutaneous melanoma (MEL), renal cell carcinoma, or another advanced or metastatic solid tumor type that is not NSCLC, UC, or MEL, are enrolled to confirm the tolerability of the RP2D of SRK-181 (determined in Part A2) and to evaluate the anti-tumor activity of SRK-181 in combination with an anti-PD-(L)1 antibody therapy. Patients in Part A1 may continue treatment with SRK-181 as a single agent at the RP2D in the LTEP following 3 cycles of treatment with SRK-181 as a single agent in Part A1. Patients in Part A2 may continue treatment with SRK-181 at the RP2D in combination with anti-PD-(L)1 antibody therapy in the LTEP following 3 cycles of treatment with SRK-181 in combination with anti-PD-(L)1 antibody therapy in Part A2. Patients in Part B may continue treatment with SRK-181 in combination with anti-PD-(L)1 antibody therapy in the LTEP following 9 cycles of treatment with SRK-181 in combination with anti-PD-(L)1 antibody therapy in Part B.

In Part A1, patients with advanced solid tumors are administered 80 mg, 240 mg, 800 mg, 1600 mg, 2400 mg, or 3000 mg of SRK-181 via intravenous infusion once every three weeks, or 2000 mg every two weeks. In Part A2, patients who are non-responders to prior anti-PD-(L)1 therapy are administered a combination of an approved anti-PD-(L)1 therapy based on tumor type and SRK-181, where SRK-181 is administered intravenously at 240 mg,

800 mg, 1600 mg, or 2400 mg once every 3 weeks. In Part B, patients who are non-responders to prior anti-PD-(L)1 therapy are administered a combination of an anti-PD-(L)1 therapy and SRK-181 via intravenous infusion. Cohort A of Part B consists of patients with non-small cell lung cancer (NSCLC) who are administered SRK-181 in combination with pembrolizumab. Cohort B of Part B consists of patients with urothelial carcinoma (UC) who are administered SRK-181 in combination with pembrolizumab. Cohort C of Part B consists of patients with melanoma (MEL) who are administered SRK-181 in combination with pembrolizumab. Cohort D of Part B consists of patients with a cancer (e.g., solid tumor, e.g., ccRCC) that is not NSCLC, UC, or MEL, and the patients are administered SRK-181 in combination with an approved anti-PD-(L)1 therapy based on tumor type. Blood chemistry assays are performed as a safety assessment in Part B, including measuring blood levels of cytokines interleukin-1 beta (IL-1β), tumor necrosis factor alpha (TNBα), interleukin-6 (IL-6), interleukin-8 (IL-8), interferon gamma (IFNγ), and interleukin 10 (IL-10).

Primary outcome measures for the study include 1) evaluating the safety and tolerability of SRK-181 (Part A1) or in combination with an anti-PD-(L)1 therapy (Part A2) and 2) determining the MTD or MAD, RP2D, and dose limiting toxicities (DLTs). DLTs response as assessed in the first 21 days of the study. MTD, MAD, RP2D, and DLTs are assessed using RECIST v1.1 and do not include toxicities clearly related to disease progression and intercurrent illness. Safety endpoints include adverse events, clinical observations (e.g., vital signs, physical examination), laboratory tests, electrocardiogram, and echocardiogram.

Secondary outcome measures of the study include evaluating the pharmacokinetics of SRK-181 alone or in combination with an anti-PD-(L)1 therapy. Specific pharmacokinetic parameters include maximum drug concentration (Cₘₐₓ), time to Cₘₐₓ (Tₘₐₓ), last validated plasma concentration (Cₗₐₛₜ), time to Clast (Tₗₐₛₜ), and half-life (T_{1/2}). Time frame for evaluating pharmacokinetic parameters are cycles 1 and 3, where each cycle is 21 days. DLT evaluation period is 14 days for the q2w regimen. The anti-tumor activity of SRK-181 alone or in combination with an anti-PD-(L)1 therapy is also evaluated as a secondary outcome. Specific clinical response parameters include objective response, defined as a complete response (CR) or partial response (PR), as determined by RECIST v1.1 or iRECIST v1.1. RECIST v.1.1 is performed during screening, 6 weeks after the first dose, every 9 weeks for the next 6 months of treatment, and every 12 weeks thereafter. Central reads are also being conducted, with a comprehensive review of the central reads to be performed once completed across the cohorts. Other secondary outcome measures include evaluating anti-drug antibodies and biomarkers. Biomarker assessment includes evaluation of the immune landscape (e.g., identify immune-inflamed, immune-excluded, and immune desert tumors) and TGFβ pathway status within the tumor. Orthogonal biomarker strategies are also applied to assess multiple biologically related pathways, such assessments include as tumor-based multiplex immunohistochemistry (IHC), next-generation sequencing, and evaluation of blood-based biomarkers.

Key inclusion criteria for the study include patients who are 18 years of age, with a predicted life expectancy of ≥3 months and patients who have measurable disease per RECIST v1.1 at screening. Additionally, patients must have an Eastern Cooperative Oncology Group performance status (PS) 0-1. To meet eligibility for part A1, patients must have a histologically documented solid tumor that is metastatic or locally advanced, for which standard of care therapy does not exist, has failed in the patient, or is not tolerated by the patient, or for which the patient has been assessed by the Investigator as not being a suitable candidate or otherwise ineligible for the standard of care therapy. To meet eligibility for Part A2 and Part B, patients must have a history of primary anti-PD-(L)1 antibody nonresponse presenting (based upon the Investigator's assessment) either as progressive disease or stable disease (e.g., not improving, but also not worsening, clinically or radiographically) after at least 3 cycles of treatment with an anti-PD-(L)1 antibody therapy (alone or in combination with chemotherapy) approved for that tumor type. Additionally, patients must also have received their most recent dose of anti-PD-(L)1 antibody therapy within 6 months of enrollment (or 9 months for UC cohort) in order to meet eligibility for Part B.

Key exclusion criteria include patients who have ECOG performance status ≥2, concurrent anti-cancer treatment, a history of active metastatic CNS disease, an active or prior history of autoimmune disease, hypersensitive or presence of anti-drug antibodies to anti-PD-(L)1 antibody therapy, and/or concurrent second malignancy. Additional exclusion criteria include patients who have second malignancy at other sites with the exceptions of adequately treated in situ carcinoma, e.g., cervical carcinoma, non-melanoma skin cancer, bilateral synchronous discordance breast cancer, or indolent prostate cancer under observation. A past history of other malignancies are allowed as long as the patient has been free of recurrence for ≥2 years, or if the patient has been treated with curative intent within the past 2 years, and in the opinion of the Investigator, is unlikely to have a recurrence. Women who are pregnant or breastfeeding are also excluded. Exclusion criteria for Part A2 and Part B include patients who are receiving concurrent anticancer treatment, with the exception of an anti-PD-(L)1 antibody therapy for Part A2 or Part B, either approved or investigational, within 28 days prior to administration of SRK-181, patients who have received biologic therapy (except for anti-PD-(L)1 antibody therapy for Part A2 or Part B), <28 days prior to administration of SRK-181, pateints who have received systemic cytotoxic chemotherapy (except for in combination with anti-PD-(L)1 antibody therapy)<28 days prior to administration of SRK-181, patients who have received targeted small molecule therapy within 5 half-lives of the compound prior to administration of SRK-181, or pateints who have a history of intolerance or treatment discontinuation due to severe irAE or other adverse reaction from prior anti-PD-(L)1 antibody therapy.

Twenty patients have been dosed (14 patients in Part A1 and 6 patients in Part A2). Dose escalation phase of the study is on-going. Early results from Part A1 showed that escalation of SRK-181 from 80 mg to 2400 mg resulted in no DLTs. Similarly, escalation of SRK-181 from 240 mg to 800 mg, when dosed in combination with an anti-PD-(L)1 therapy, did not result in DLTs in Part A2. A dose of 3000 mg of SRK-181 is currently under evaluation in Part A2.

Early results are as follows.

As of June 2021, 25 patients have been enrolled in Part A with a median of 4 prior lines of therapies (range 1-9). The cancer types include colorectal, ovarian, prostate, and primary unknown in Part A1 and liver, melanoma, NSCLC, oropharynx, renal cell carcinoma (RCC) and uterine in Part A2. In Part A1, 15 patients were treated with SRK-181 monotherapy at doses of 80, 240, 800, 1600, 2400, 3000 mg Q3W, and no dose limiting toxicity (DLT) was observed. The last cohort (2000 mg Q2W) is still under evaluation. The most common treatment-related AEs (TRAE, >10%) of any grade were decreased appetite and fatigue (each: 13.3%, n=2). Six patients had best response of stable disease (SD) (2/colorectal cancer, 1/prostate cancer, and 3/ovarian cancer). Three ovarian cancer patients were stable ≥153 days with tumor regressions. In Part A2, 10 patients were treated with SRK-181 at doses of 240, 800 and 1600 mg Q3W+pembrolizumab. No DLT was observed up to 800 mg. Cohorts of 1600 mg Q3W is under evaluation. Additional two cohorts are planned. The most common TRAE (>10%) of any grade was rash maculo-papular (20%, n=2). One RECIST1.1 partial response (PR) was observed (800 mg) in an anti-PD-1 resistant RCC patient and 2 patients had best response of SD (1/oropharynx cancer, 1/liver cancer). The half-life of SRK-181 ranged from 3.9 to 19.3 days across the doses tested. It is therefore concluded that SRK-181 is well tolerated as monotherapy and in combination with anti-PD-(L)1 at dose levels tested to date. One PR was observed in a RCC patient who had previously failed anti-PD-1 treatment.

As of Sep. 7, 2021, 29 patients have been dosed as follows.

| Category | Part A1 | | Part A2 | |
|---|---|---|---|---|
| N | 19 | | 10 | |
| Age, median (range) | 66.0 | (41, 79) | 62.5 | (32, 75) |
| Gender (F/M) | 8/11 | | 1/9 | |
| Ethnicity | | | | |
| Hispanic or Latino | 0 | | 2 | |
| Not Hispanic or Latino | 18 | | 8 | |
| Not Reported | 1 | | 0 | |
| Race | | | | |
| White | 19 | | 10 | |
| Prior Lines of Therapy, median (range) | 4 | (1, 9) | 4 | (2, 6) |

Patient disposition was as follows.

| Category | Part A1 | Part A2 |
|---|---|---|
| Enrolled | 19 | 10 |
| On Study | 3 | 1 |
| Stopped Treatment | 16 | 9 |
| Reason for Completion/Discontinuation | | |
| Adverse Event* | 1 | 2 |
| Clinical Progression | 3 | 3 |
| Disease Progression based on RECIST v1.1 | 11 | 3 |
| Investigator Decision | 1 | 0 |
| Withdrawal of Consent | 0 | 1 |

*3 patients discontinued from the study due to AEs deemed unrelated to SRK-181 treatment.

As of Oct. 12, 2021, no DLTs were observed up to 3000 mg q3w and 2000 mg q2w (every 2 weeks) in Part A1; and up to 1600 mg q3w (every 3 weeks) in Part A2. The recommended Part B dose is 1500 mg q3w and/or 1000 mg q2w. In the mouse preclinical efficacy model, the observed Cave (average plasma concentration) for SRK-181 was about 80 μg/mL at 10 mg/kg. The recommended Part B dose of 1500 mg q3w has been chosen based on the ability to attain Ctrough (trough concentration) greater than or equal to about 80 μg/mL at the lower bound of the 90% Cl. The dose of 1000 mg q2w was chosen since it allows for equivalent Cave exposure to 1500 mg q3w, which would provide flexible dose frequency when combining with different anti-PD-(L)l in Part B.

Treatment-emergent AEs, all grades greater than or equal to 20% in Part A1 are shown below.

| | Dose (mg) | | | | | | | |
|---|---|---|---|---|---|---|---|---|
| | 80 | 240 | 800 | 1600 | 2400 | 3000 | 2000 | All |
| | (n = | (n = | (n = | (n = | (n = | (n = | (n = | (n = |
| | 1) | 1) | 3) | 4) | 3) | 3) | 4) | 19) |
| Decreased | 1 | 0 | 1 | 1 | 1 | 0 | 1 | 5 (26.3%) |
| appetite | | | | | | | | |
| Fatigue | 1 | 1 | 0 | 0 | 2 | 0 | 1 | 5 (26.3%) |
| Abdomi- | 0 | 0 | 0 | 1 | 1 | 1 | 1 | 4 (21.1%) |
| nal pain | | | | | | | | |
| ALT | 0 | 0 | 1 | 0 | 1 | 1 | 1 | 4 (21.1%) |
| increased | | | | | | | | |
| AST | 0 | 0 | 1 | 0 | 1 | 1 | 1 | 4 (21.1%) |
| increased | | | | | | | | |
| Back pain | 0 | 0 | 1 | 0 | 0 | 2 | 1 | 4 (21.1%) |
| Nausea | 1 | 0 | 0 | 1 | 0 | 0 | 2 | 4 (21.1%) |
| Vomiting | 0 | 0 | 0 | 0 | 1 | 1 | 2 | 4 (21.1%) |

All dose levels were administered q3w except 2000 mg, which was administered q2w. Clinical cutoff date: Sep. 7, 2021.

Treatment-emergent AEs related to SRK-181, all grades, greater than 10% for Part A1 are shown below.

| | Dose (mg) | | | | | | | |
|---|---|---|---|---|---|---|---|---|
| | 80 | 240 | 800 | 1600 | 2400 | 3000 | 2000 | All |
| | (n = | (n = | (n = | (n = | (n = | (n = | (n = | (n = |
| | 1) | 1) | 3) | 4) | 3) | 3) | 4) | 19) |
| Fatigue | 0 | 1 | 0 | 0 | 1 | 0 | 1 | 3 (15.8%) |
| Decreased | 1 | 0 | 1 | 0 | 0 | 0 | 0 | 2 (10.5%) |
| appetite | | | | | | | | |
| Nausea | 1 | 0 | 0 | 0 | 0 | 0 | 1 | 2 (10.5%) |

All dose levels were administered q3w except 2000 mg, which was administered q2w. Clinical cutoff date: Sep. 7, 2021.

Treatment-emergent AEs, all grades greater than or equal to 20% for Part A2 are shown below.

| | Dose (mg) | | | |
|---|---|---|---|---|
| | 240 | 800 | 1600 | All |
| | (n = 3) | (n = 3) | (n = 4) | (n = 10) |
| Dyspnoea | 0 | 1 | 2 | 3 (30%) |
| Blood creatinine increased | 0 | 1 | 1 | 2 (20%) |
| Hypokalaemia | 0 | 2 | 0 | 2 (20%) |
| Hypomagnesaemia | 1 | 1 | 0 | 2 (20%) |
| Rash maculo-papular | 1 | 1 | 0 | 2 (20%) |

All dose levels were administered q3w. Clinical cutoff date: Sep. 7, 2021.

Treatment-emergent AEs related to SRK-181/Anti-PD(L)1, all grades, greater than 10% for Part A2 are shown below.

| | Dose (mg) | | | |
|---|---|---|---|---|
| | 240 | 800 | 1600 | All |
| | (n = 3) | (n = 3) | (n = 4) | (n = 10) |
| Rash maculo-papular | 1 | 1 | 0 | 2 (20%) |

All dose levels were administered q3w. Clinical cutoff date: Sep. 7, 2021.

Treatment-related Grade 3 AEs were alanine aminotransferase increased (1 patient in Part A1); lipase increased, hypoxia and rash maculo-papular (1 patient each in Part A2); no Grade 4 or 5 treatment-related AEs occurred. Treatment-related SAE of elevated troponin I (1 patient) was observed in Part A1 (at 2000 mg a2w); no SRK-181 treatment-related SAEs were observed in Part A2.

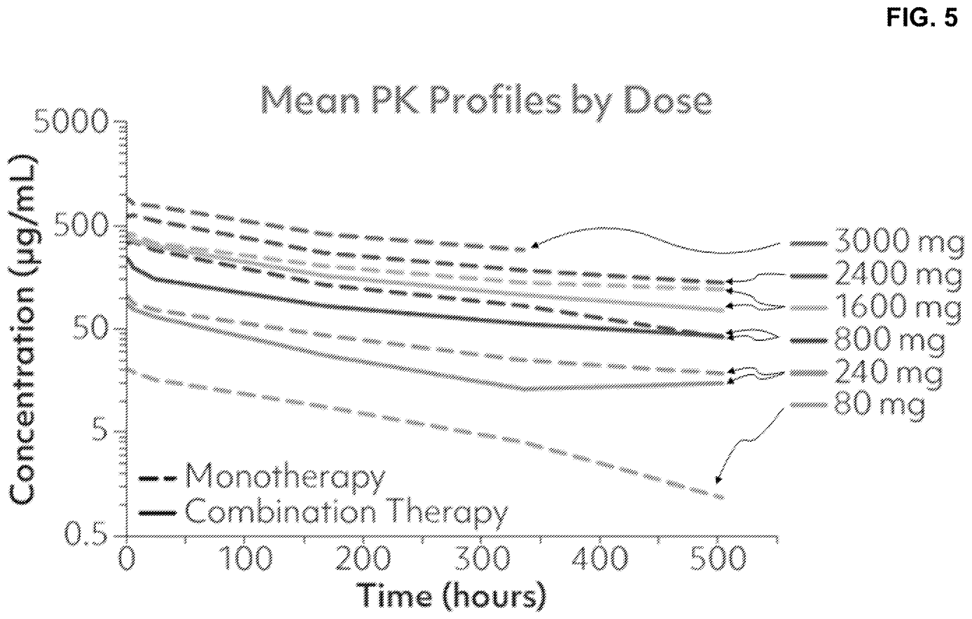

FIG. 5 provides the mean pharmacokinetic (PK) profiles by dose. SRK-181 displayed typical monoclonal antibody PK characteristics, with observed CLss (clearance at steady state) that ranged from 0.0164 to 0.0225 L/h and Vss (volume distribution at steady state) that ranged from 4.21 to 6.85 L. Based on a power model, dose-proportional PK was observed for SRK-181. The T1/2 of SRK-181 was 5.4 to 10.7 days.

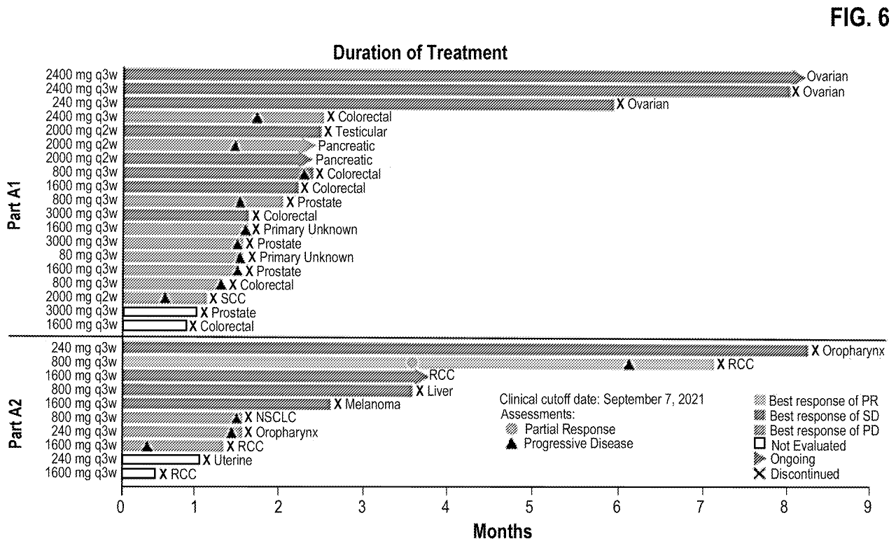

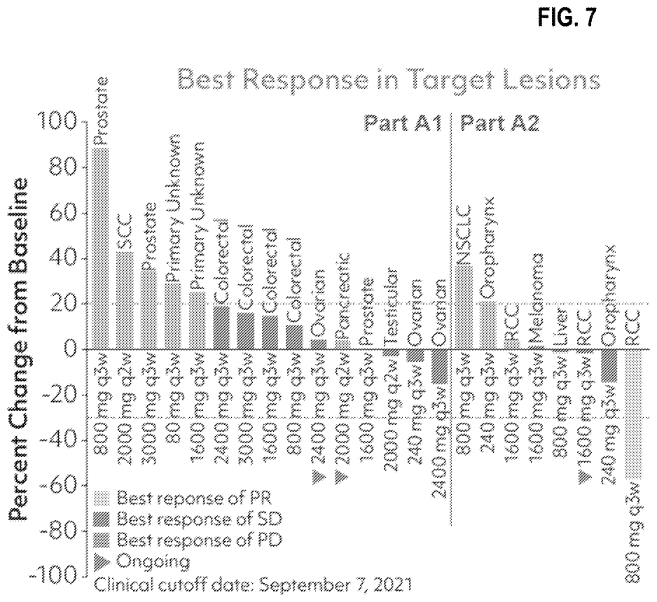

FIG. 6 provides the preliminary efficacy results based on the duration of treatment, and FIG. 7 depicts the best response in target lesions. Among 19 patients in Part A1, 8 patients had a best response of stable disease (SD) (3/ovarian, 3/colorectal, 1/pancreatic, 1/testicular), and 3 ovarian cancer patients were stable for greater than or equal to 153 days with tumor regressions. Among 10 patients in Part A2, at 800 mg q3w, one confirmed RECIST1.1 PR (partial response) was observed in a patient with anti-PD-1-resistant RCC. 4 patients had best response of SD (1/oropharynx, 1/liver, 1/melanoma, 1/RCC), and one oropharynx cancer patient was stable for 245 with tumor regressions.

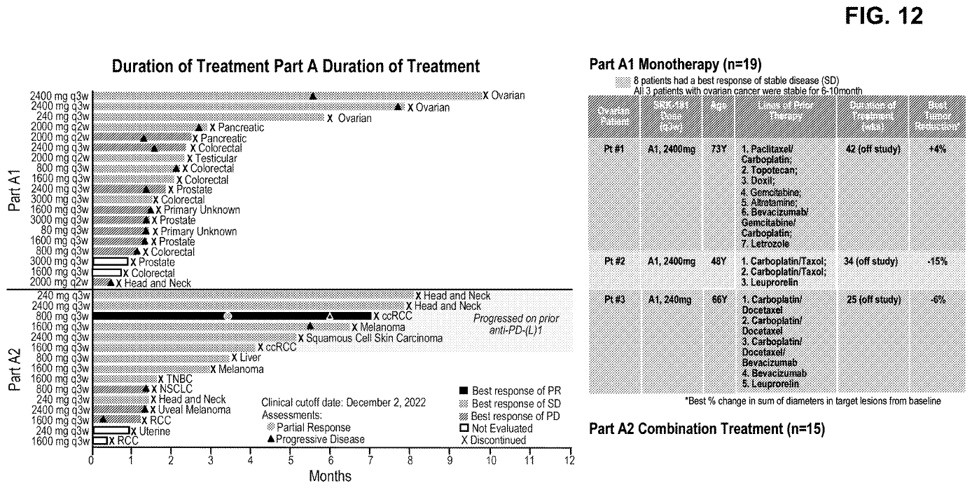

Thus, as of Sep. 7, 2021, SRK-181 has been well-tolerated both as a monotherapy and in combination with anti-PD-(L)1. The recommended Part B dose was determined to be 1500 mg q3w and/or 1000 mg q2w. No DLT was observed up to 3000 mg q2w and 2000 mg q2w with SRK-181 monotherapy. No DLT was observed up to 1600 mg q3w with SRK-181+pembrolizumab combination treatment. The next planned dose in Part A2 will be 2400 mg q3w. SRK-181 efficacy will be evaluated in Part B, which was initiated on Oct. 12, 2021. An updated evaluation of Parts A1 and A2 is reported in FIG. 12.

### Example 4: Prevalence of Tumor MDSC in Various Solid Tumors

A signal intensity filter for detecting cell surface markers of human MDSCs was established. Multiple markers were selected and developed to distinguish MDSC subtypes from other monocytes. Cell surface markers for distinguishing human MDSCs include CD11 b, CD33, CD66b, CD14, CD15, and HLA-DR. A chromogenic assay was performed to define the immunofluorescence dynamic range of each antibody used to detect the cell surface markers. Two types of signal intensity filters were used to identify MDSCs: 1) binary intensity selection, i.e., based on positive vs. negative signal, e.g., distinguishing CD14+ mMDSC from CD15+ gMDSC; and 2) categorical binning of signal intensities to define a range of signal intensities, e.g., to distinguish HLA-DR^{lowIo-neg}mMDSC from HLA-DR^{neg}gMDSC. Signal intensity filters were applied sequentially, and categorization was reviewed and confirmed by pathologists. Exemplary thresholds for detecting cell surface markers after application of signal intensity filters are shown in Tables 14A-C below.

TABLE 14A

| | HLADR | CD33 |
|---|---|---|
| Negative | <0.105 | <0.17 |
| Low | 0.105-0.125 | 0.17-0.18 |
| Med | 0.125-0.155 | 0.18-0.19 |
| High | >0.155 | >0.19 |

TABLE 14B

| | CD11b | CD14 | CD15 | CD66b |
|---|---|---|---|---|
| Negative | <0.145 | <0.24 | <0.19 | <0.19 |
| Positive | >0.145 | >0.24 | >0.19 | >0.19 |

TABLE 14C

| | CD11b | HLADR | CD14 | CD15 | CD33 | CD66b |
|---|---|---|---|---|---|---|
| gMDSC | + | − | − | + | +/low | + |
| mMDSC | + | −/low | + | − | +/high | − |
| classical | + | + | + | − | + | − |
| monocyte | | | | | | |
| neutrophil | + | −/med | − | + | +/med | + |
| m1 macrophage | + | +/high | − | − | + | − |
| m2 macrophage | + | + | − | − | + | − |

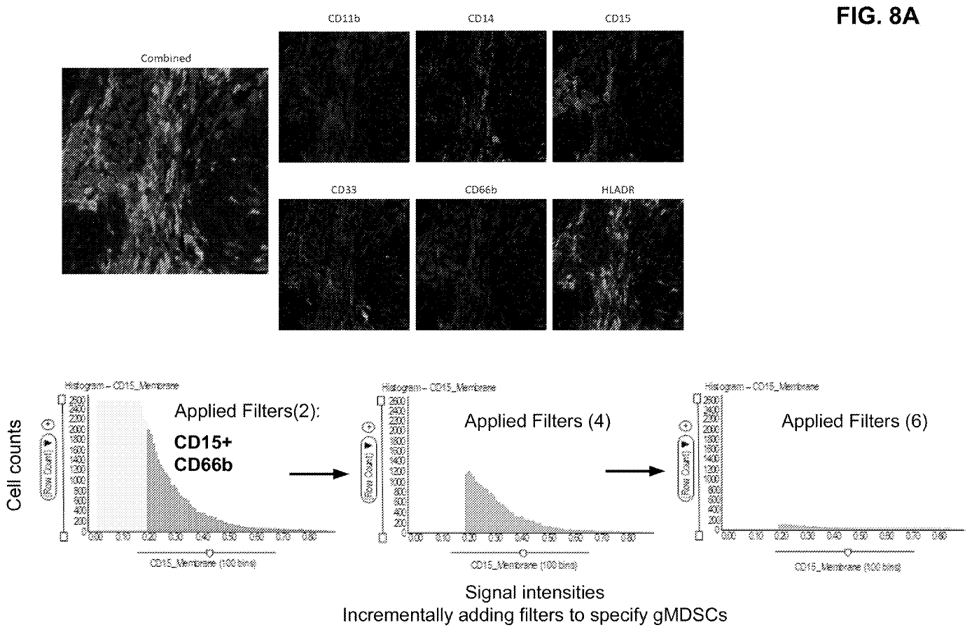

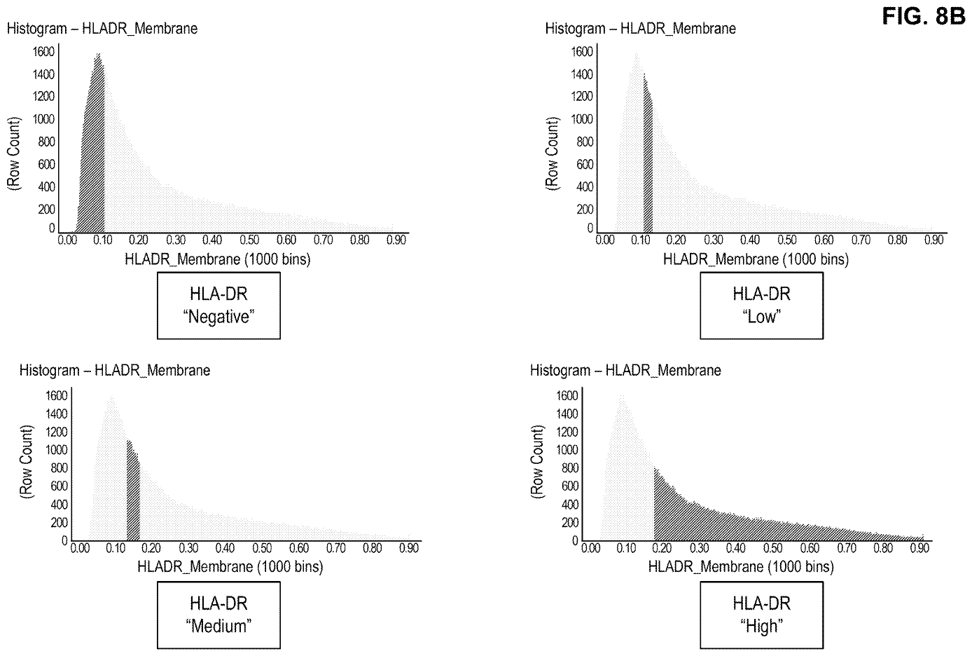

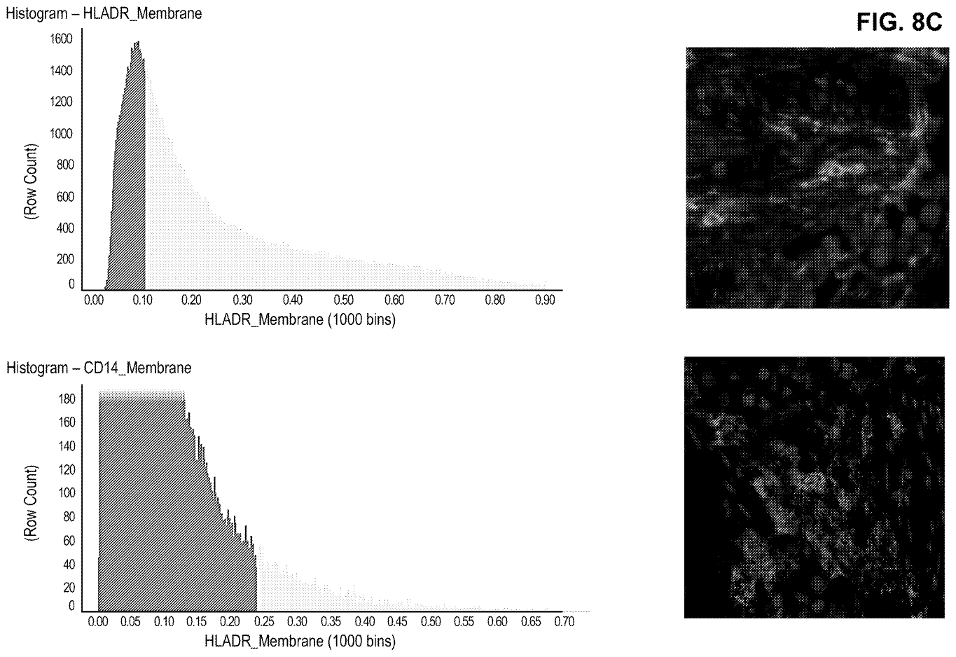

An exemplary analysis of signal intensity filtering of MDSC markers in bladder cancer is shown in FIG. 8A. An example of HLA-DR detection using threshold cutoffs is shown in FIG. 8B. All cells were plotted to illustrate threshold cuttoffs, where the x-axis shows staining intensity (binned columns) and the y-axis shows the number of cells. Visualization of signal intensities were normalized by channel in order to reduce background and bleed through (spectral unmixing). Because of this, threshold cutoffs varied from channel to channel. Examples of signal normalization are shown in the panels of FIG. 8C.

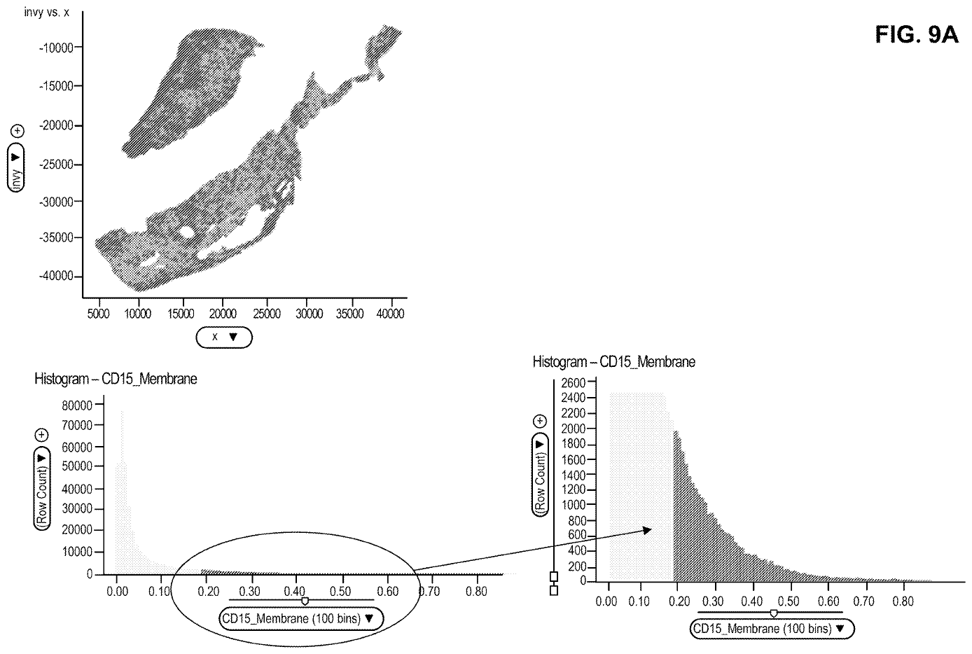

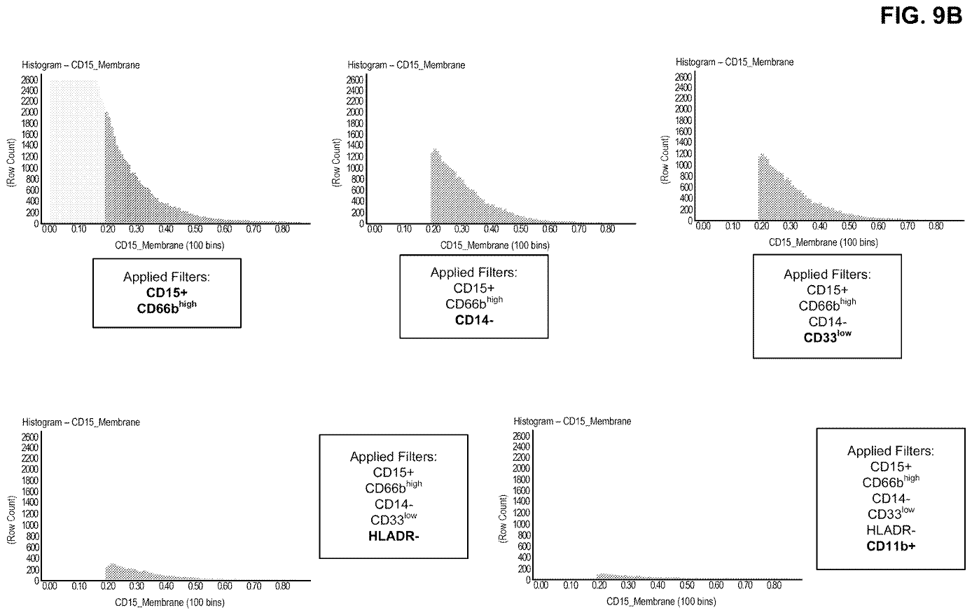

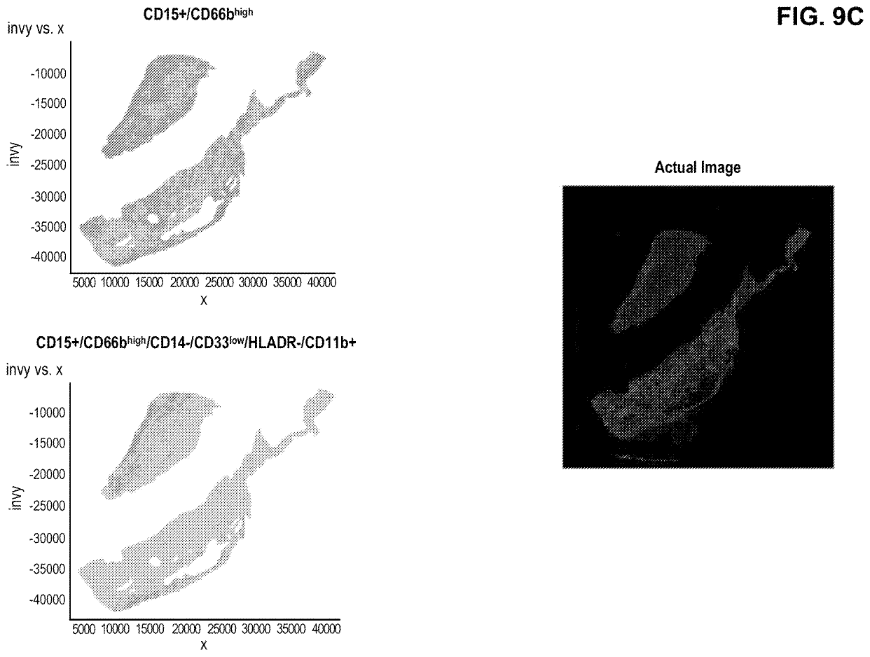

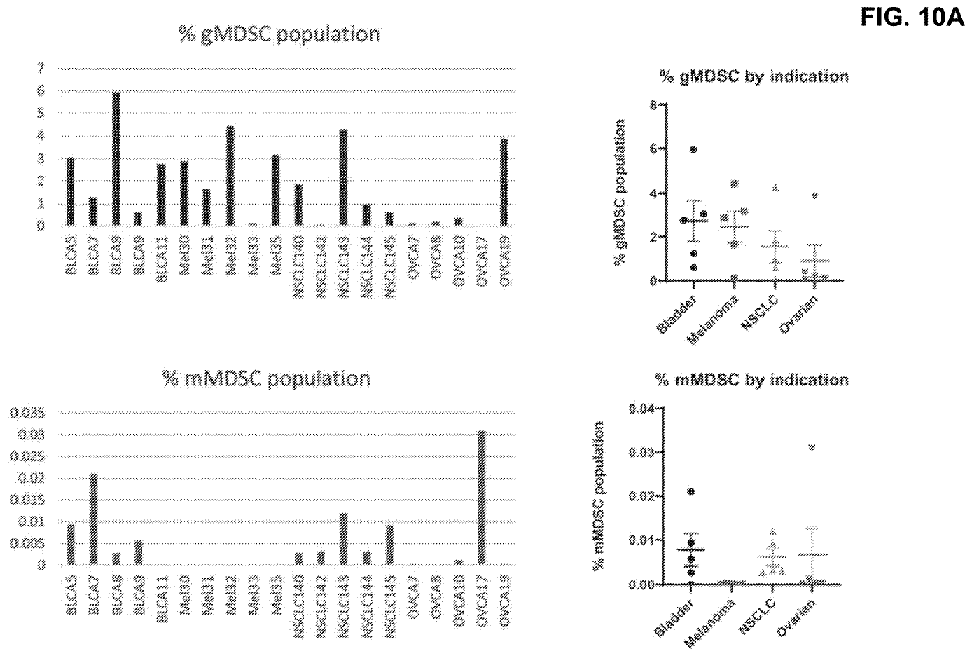

gMDSC populations in tumor samples were identified by first filtering for CD15 and CD66b positivity (FIG. 9A). This filtering distinguished gMDSCs from mMDSCs, which are CD15 and CD66b negative. Sequential application of signal filters was then applied to specify gMDSCs according to the markers and thresholds described above (FIG. 9B). A comparison of the original tumor image from a bladder cancer sample (right panel), without signal filtering, the image after applying CD15+/CD66b^{high} filtering (top left panel), and the image after applying CD15⁺/CD66b^{high}/CD14⁻/CD33^{low}/HLADR⁻/CD11b⁺ filtering (bottom left panel) is shown in FIG. 9C. A large portion of the gMDSC population appeared to be at the tumor edge, near the stromal compartment.

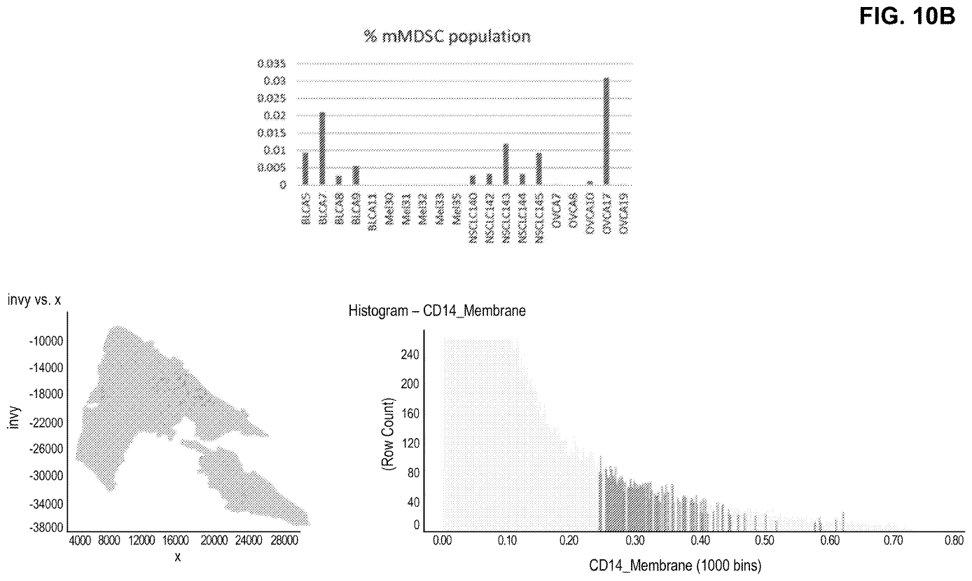

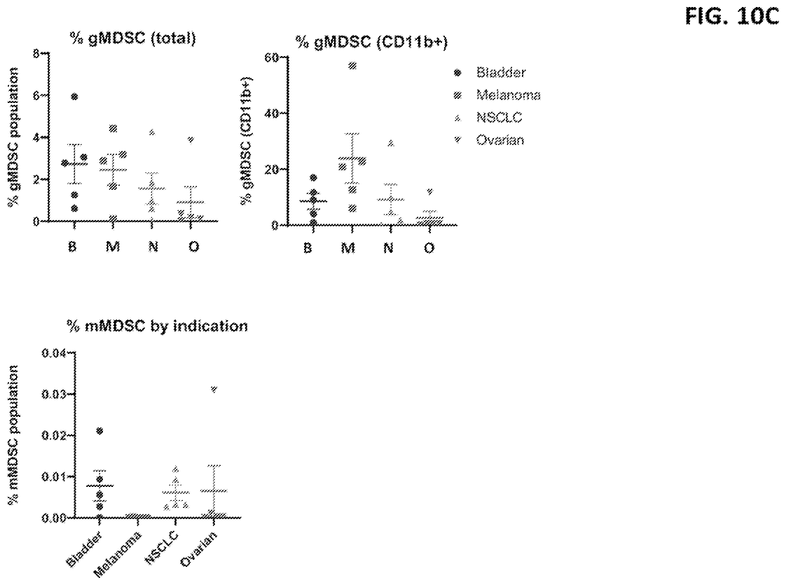

Using the same signal filtering described above, gMDSC and mMDSC populations were analyzed in samples from bladder cancer, melanoma, NSCLC, and ovarian cancer (FIGS. 10A-C). The samples had much higher gMDSC populations as compared to mMDSC populations. Ovarian cancer exhibited the lowest gMDSC levels and the highest mMDSC levels among the various indications examined. mMDSC levels were the lowest in melanoma samples.

### Example 5: Safety, Pharmacokinetics, Efficacy, and Biomarker Results of SRK-181 (a Latent TGFβ1 Inhibitor) from a Phase I Trial (DRAGON Trial)

SRK-181 is a fully human, IgG4 monoclonal antibody capable of selectively binding to latent transforming growth factor-beta 1 (TGFβ1), which has been associated with the primary resistance to immune checkpoint inhibitors (e.g., PD-1/PD-L1 [PD-(L)1] blockade). Compared to broad TGFβ inhibitors, SRK-181 was observed to have improved safety profile (no cardiotoxicities) in four-week GLP nonclinical toxicology studies. More specifically, weekly dosing of up to 200 mg/kg of SRK-181 in rats showed minimal toxicities with findings consistent with normal variations among rats and no test article-related toxicities. The NOAEL in rats for SRK-181 was 200 mg/kg QW, the highest dose evaluated. Similarly, the NOAEL in non-human primates was 300 mg/kg, the highest dose evaluated.

DRAGON trial (NCT04291079) is a multicenter ongoing Phi study with a 3+3 dose escalation design to evaluate SRK-181 in pts with advanced solid tumors at 80-3000 mg q3w and 2000 mg q2w (Part A1), and SRK-181+anti-PD-(L)1 in pts with no response to prior anti-PD-(L)1 therapy at 240-2400 mg q3w (Part A2). In Part B (expansion phase), SRK-181 (1500 mg q3w)+ anti-PD-(L)1 were administered in anti-PD-(L)1-resistant pts with non-small cell lung cancer (NSCLC), urothelial carcinoma (UC), melanoma (MEL); and clear cell renal cell carcinoma (ccRCC) and head and neck squamous cell carcinoma (HNSCC) pts with disease progression on the most recent prior anti-PD-1.

This study enrolled 19 pts in Part A1 and 15 in Part A2 with no DLT observed. The median prior lines of therapies was 4 (range 1-10). In Part A1, the most common treatment-related AEs (TRAEs, >10%) of any grade were fatigue (16%, n=3), decreased appetite and nausea (each: 11%, n=2). Best response of stable disease (SD) was observed in 8 pts. All 3 ovarian cancer pts were stable for 25 to 42 weeks. In Part A2, the TRAEs (>10%) of any grade were pruritus, rash and rash maculo-papular (each: 20%, n=3), diarrhea (13%, n=2). One confirmed RECIST1.1 partial response (cPR) was observed at 800 mg in a pt with anti-PD-1 resistant RCC who stayed on study for 30 weeks. Nine pts had best response of SD and 6 of them were stable >16 weeks. SRK-181 treatment resulted in increased levels of circulatory TGFβ1, which suggested target engagement. One patient with Head and Neck cancer achieved a 29.4% tumor reduction. As of Jan. 12, 2024, 72 pts (including 29% females, median age of 65) were enrolled in Part B. All patients received at least one anti-PD-1 therapy, with a median of 3 prior lines of therapy (range 1-9). All ccRCC and all but 2 HNSCC patients had a best response of SD or PD in prior anti-PD-1 treatment. All these patients had progressed on the most recent anti-PD-1 treatment. None of the MEL, UC, and NSCLC patients had a response to prior anti-PD-1 treatment. Biomarker analyses in Part B included immunohistochemistry and flow cytometry.

SRK-181 has been tolerated as monotherapy and in combination with anti-PD-(L)1. No DLT was observed up to 3000 mg q3w/2000 mg q2w as monotherapy and up to 2400 mg q3w as combination treatment. Early evidence of efficacy was observed with 3 cPR in pts in anti-PD-1 resistant ccRCC.

Preliminary Biomarker Data of SRK-181 from a Phase/Trial (DRAGON Trial)

A biomarker strategy to validate SRK-181's mechanism in patients and evaluate treatment response was based on the proof of concept for SRK-181 in preclinical models (as discussed above). SRK-181 (1500 mg q3w)+pembrolizumab was administered to anti-PD-1-resistant patients with urothelial carcinoma (UC), melanoma, non-small cell lung cancer (NSCLC), clear cell renal cell carcinoma (ccRCC), or head and neck cancer (no data available for head and neck patients). Biopsies were collected at baseline and between post-treatment day 21 and 45. Biopsies were formalin-fixed, paraffin-embedded, and stained using a chromogenic assay for CD8. CD8-stained sections were first analyzed in a primary compartmental analysis that quantified CD8+ T cells within tumor, tumor margin, and stromal compartments. CD8+-stained sections were then analyzed using tumor nest analysis. Tumor nests were defined as containing at least 250 cells and at least 500 μm². Nests were characterized by CD8+ T cell content using the following criteria: >5% CD8+ in tumor nest (Infiltrated); <5% CD8+ in tumor nest, >1% CD8+ in peripheral interface (Excluded); <5% CD8+ in tumor nest and <1% CD8+ in peripheral interface (Desert); % Tumor=nest cell #/total tumor compartment cell #×100. Blood for flow cytometry analysis of myeloid-derived suppressor cells (MDSCs) was originally collected at baseline and pre-infusion C2D1 (cycle 2 day 1). After protocol amendment, blood was collected every Cycle Day 1 pre-infusion and at end-of-cycle visit.

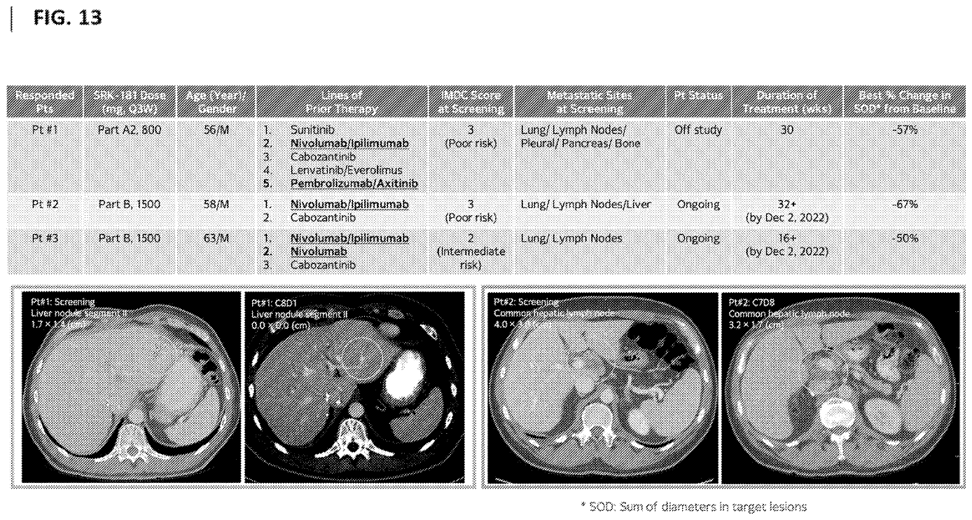

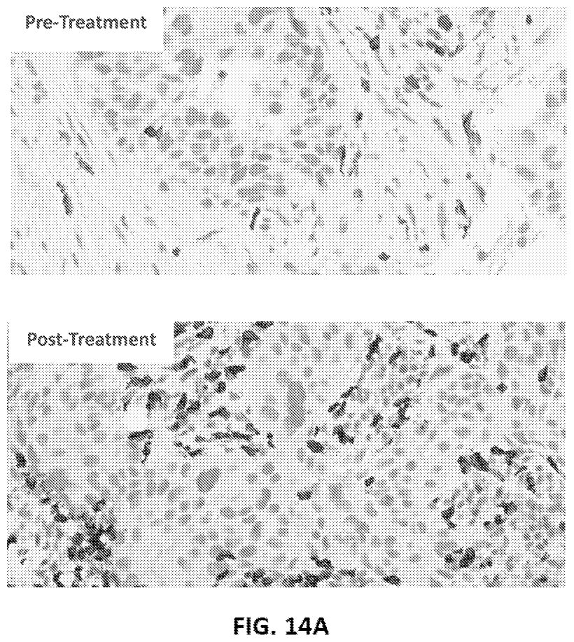

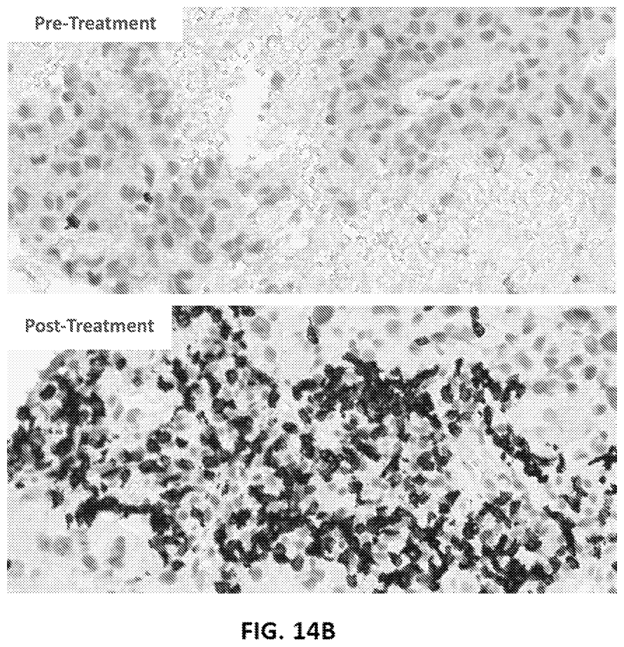

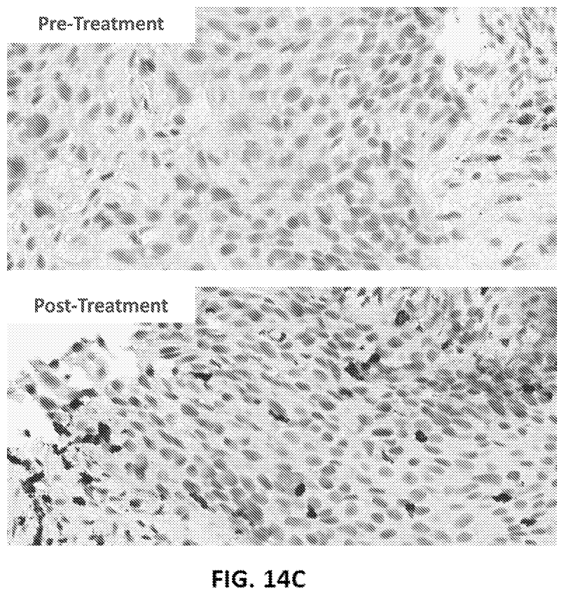

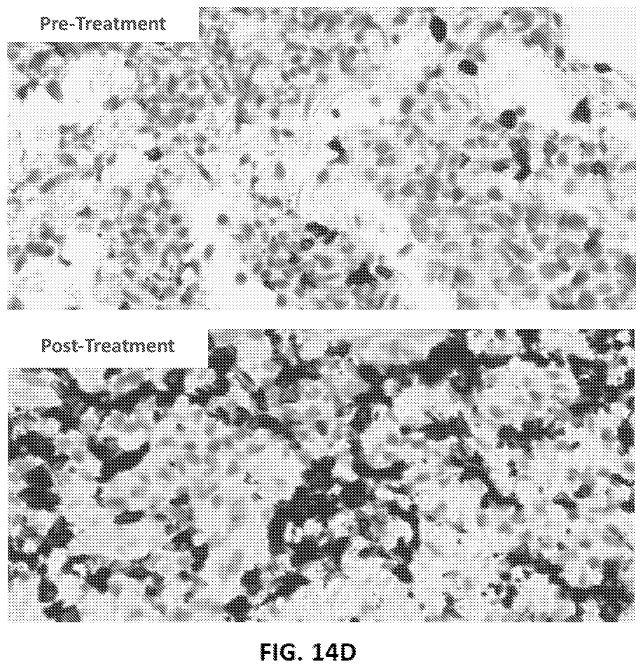

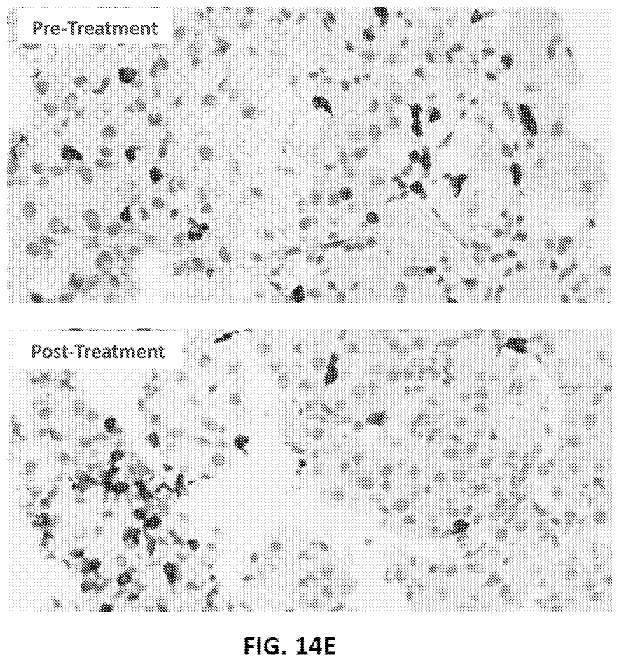

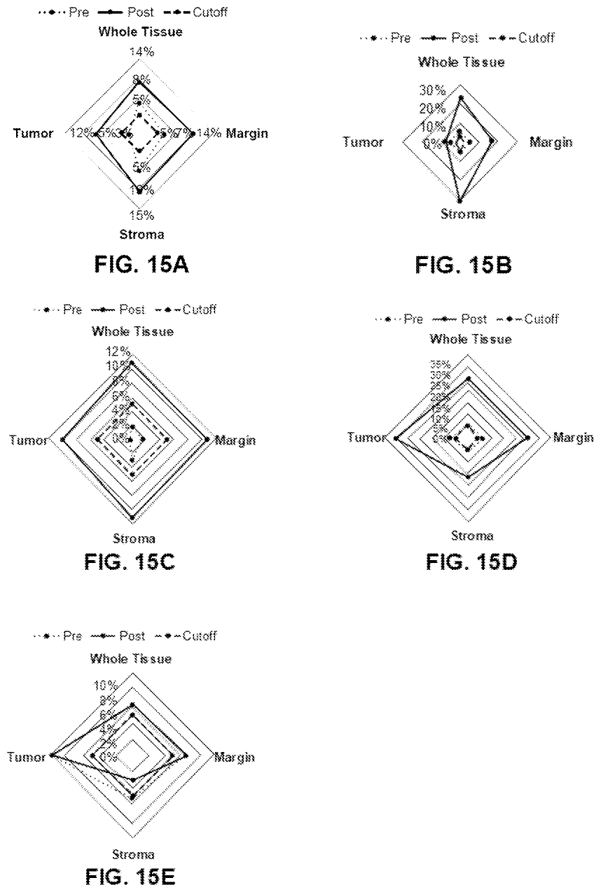

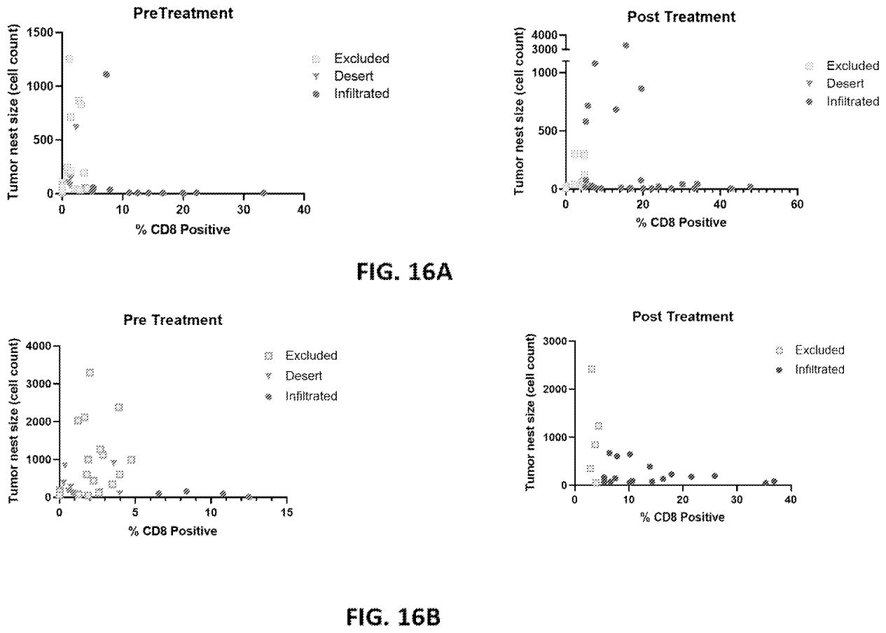

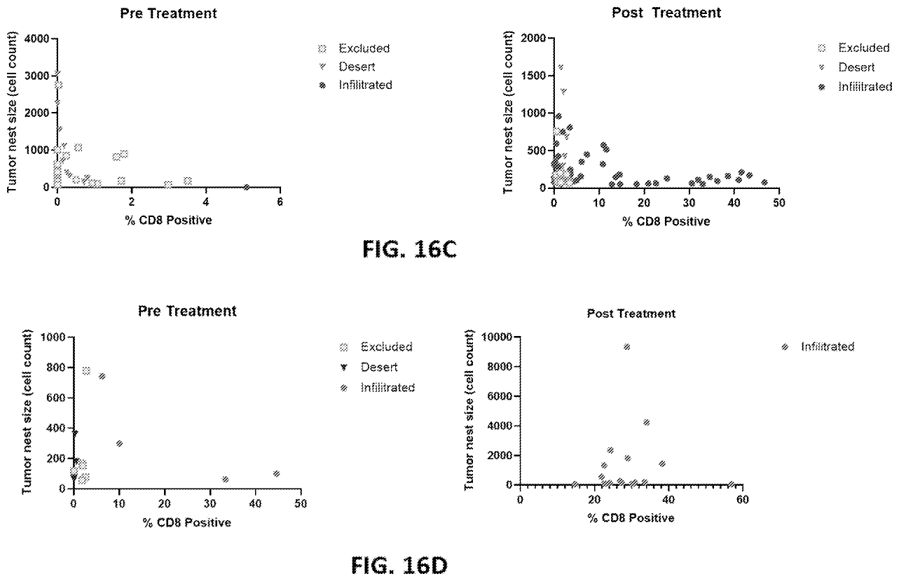

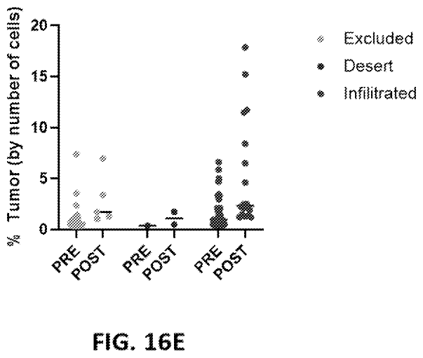

Representative pre- and post-treatment paired biopsies (CD8-stained) from UC, melanoma, NSCLC, and ccRCC patients are shown in FIGS. 14A-E. For each paired biopsy, corresponding primary compartmental analysis of % CD8+ T cells per compartment is shown in FIGS. 15A-E and tumor nest analysis is shown in FIGS. 16A-E. In the UC, melanoma, and NSCLC patients, an increase in CD8+ T cell infiltration was observed in the post-treatment biopsies, indicating that the SRK-181+pembrolizumab therapy results in more infiltrated tumor nests, overcoming an initially excluded or desert phenotype. Similar results were seen in the one other UC patient and the one other melanoma patient (data not provided). FIG. 13 reports data from ccRCC patients who achieved PR in response to a combination treatment of SRK-181 and an anti-PD-1, including two sets of images represent pre- and post-treatment collected from patient 1 and patient 2.

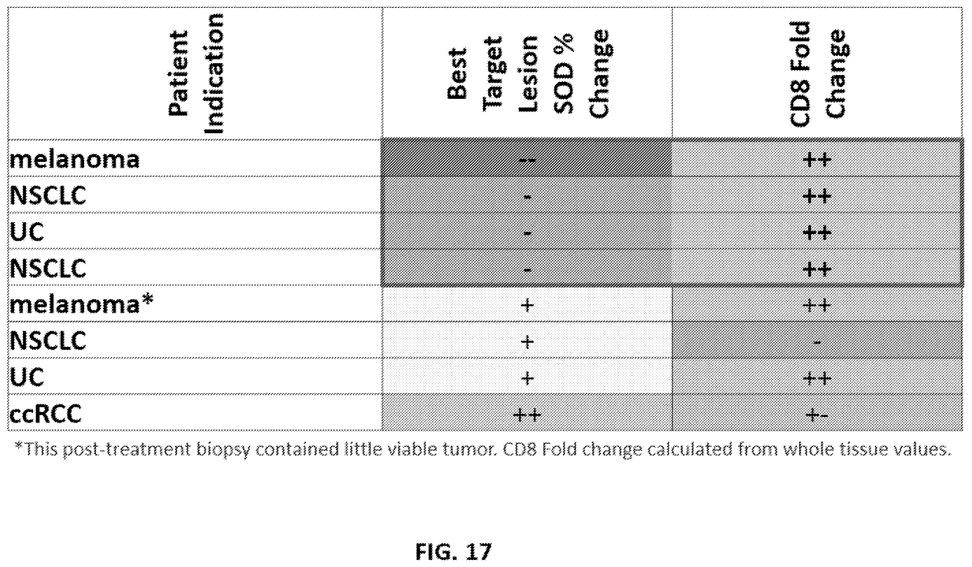

These data show that SRK-181 treatment leads to increased CD8+ T cell infiltration into tumors across multiple different tumor types: i.e., UC, melanoma, and NSCLC. This is consistent with the mechanism of action observed in preclinical studies (as shown in Martin et al. Science Translational Medicine (2020), 12(536): eaay8456). An increase in CD8+ T cell infiltration into the tumor compartments also correlated with tumor shrinkage (see FIG. 17). Indeed, in these tumor types, all patients that were observed to experience tumor shrinkage also had an infiltration of CD8+ T cells into the tumor compartments.

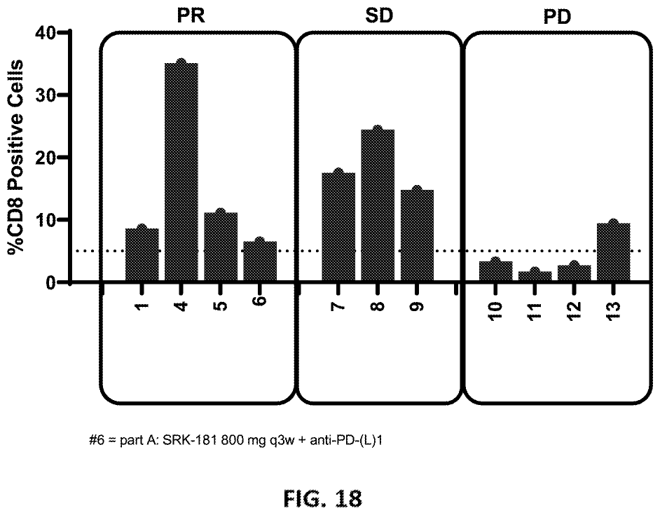

While the representative ccRCC biopsies did not show a significant increase in CD8+ T cell infiltration, the collection of paired biopsies from ccRCC patients was challenging since several post-treatment biopsies did not contain sufficient viable tumors for analysis. Additionally, in the majority of RCC patients (8/11), baseline (pre-treatment) CD8+ cell levels indicated that the ccRCC tumors already had an infiltrated phenotype, despite being non-responsive to anti-PD(L)1 therapy (as shown in FIG. 18). Nevertheless, as shown in FIG. 16E, there was a slight increase in CD8+ cell infiltration by tumor nest analysis in the representative ccRCC biopsies.

In addition to increasing CD8+ cell infiltration, preclinical studies also suggest that SRK-181 reduces MDSC levels within the tumor microenvironment and in circulation. MDSC level may correlate with clinical response, since MDSCs play a critical role in tumor development and resistance to immunotherapy due to their immune suppressive functions and promotion of tumor growth. MDSCs may also contribute to resistance to immunotherapy. Accordingly, circulating MDSC levels were regularly assessed in all patient groups. The level of gMDSC cells was assessed in patient blood samples using flow cytometry (MDSCV Assay, Labcorp, Burlington, NC). The level of mMDSC cells was also assessed in patient blood samples using flow cytometry (MDSCV Assay, Labcorp, Burlington, NC). Multiple sample points were collected on Day 21, but the other timepoints have only one sample because the protocol was amended later to add sample collections on the first day of each subsequent cycle.

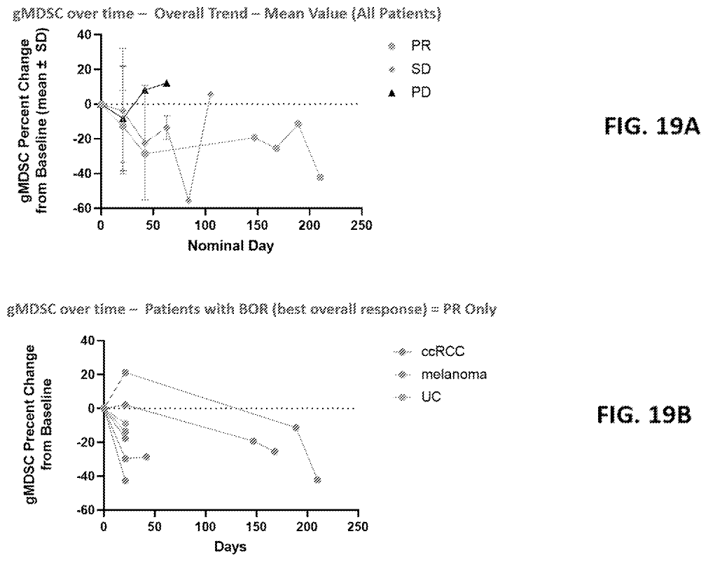

For patients with the best response (“PR” in FIGS. 19A and 19B), treatment with SRK-181+pembrolizumab decreased circulatory gMDSCs below baseline (FIGS. 19A and 19B). A decrease in circulatory mMDSCs was also observed. Partial Responses (PRs) have been observed in ccRCC, melanoma, and UC patients, and all PR patients also experienced a decrease in circulating gMDSC. Achieving PR was not associated with decreased circulatory mMDSC below baseline. For patients with the best response of PD, mMDSC levels increased above baseline.

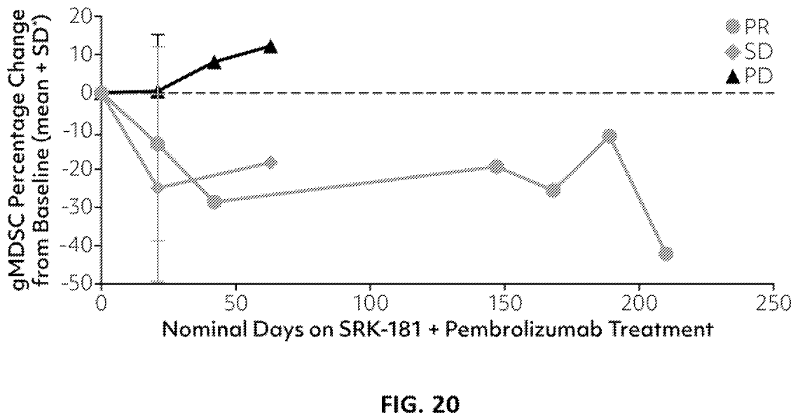

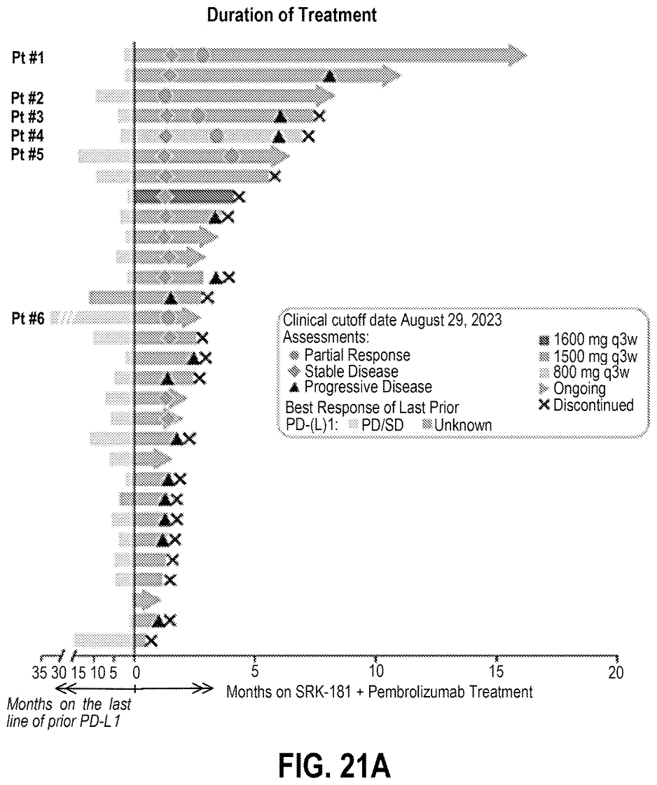

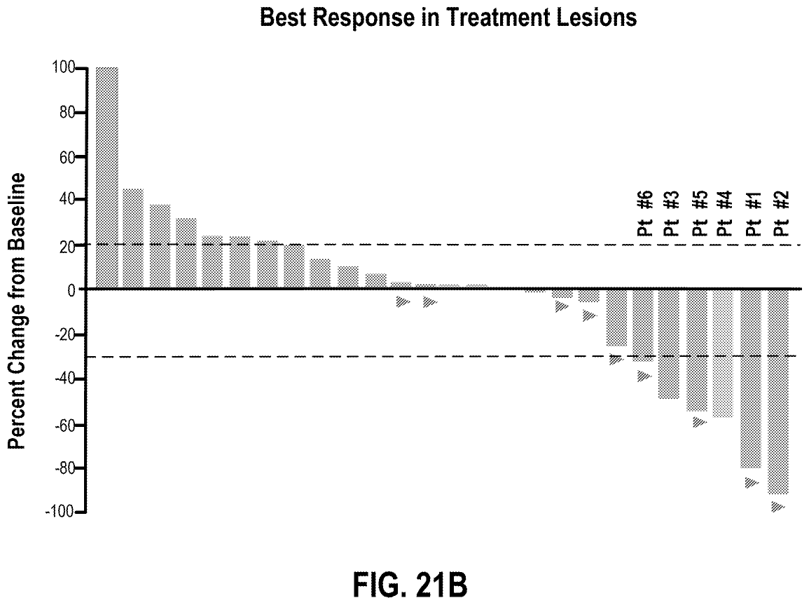

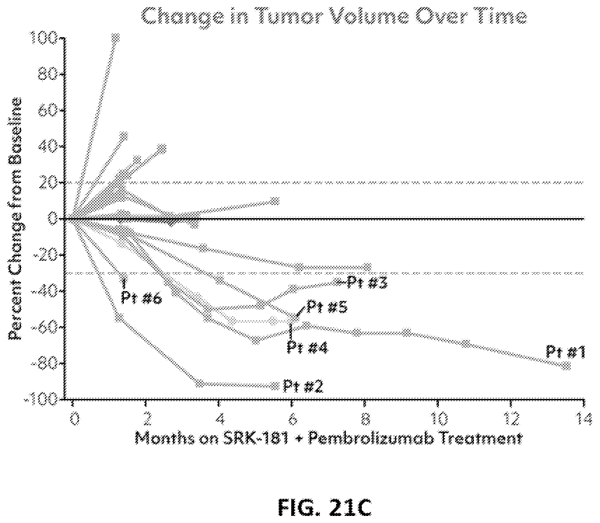

The correlation between suppression of gMDSCs and clinical response was especially clear in ccRCC patients (FIG. 20). Of the ccRCC cohort (n=28), 6 patients (range of 2-5 prior lines of therapy) had confirmed partial response (PR) based on RECIST 1.1 criteria by PI assessment (ORR=21.4%, disease control rate=57%). The 6 PR patients achieved a best tumor reduction of −33% to −93%, and remained on study for 2.8+ to 16.3+ months, and 4 PR patients remain on study. 10 patients had stable disease (SD). These patients remained on study for 2+ to 11.1+ months. Five SD patients are still on study. FIGS. 21A-C provides an overview of duration of treatment, best response in target lesions, and change in tumor volume over time for the 28 patients enrolled in the ccRCC cohort to date. Reductions in circulating gMDSC levels were found to correlate with better clinical responsiveness in ccRCC patients treated with SRK-181+pembrolizumab.

Overall, these data support the hypothesis that SRK-181 improves anti-tumor immunity in cancer patients, by increasing CD8+ T cell infiltration into solid tumors and/or by reducing the levels of immunosuppressive cells, such as MDSCs, especially gMDSCs. This improved anti-tumor immunity may improve outcomes in patients with tumors that are resistant or refractory to checkpoint inhibitor therapies, even in cancers that have an immune-infiltrated phenotype prior to treatment with SRK-181.

In Part B, the most common treatment-related AEs (TRAE, >10%) of any grade were rash (23.6%), pruritus (20.8%), fatigue (19.4%), and diarrhea (12.5%). Grade 3 TRAE (>5%) were rash (8.3%). Only 1 grade 4 TRAE (dermatitis exfoliative generalised) occurred. No grade 5 TRAE and no treatment-related SAE (>2% [1 pt]) was observed. Efficacy results are presented in the table below. Tumor measures were based on RECIST 1.1 criteria by PI assessment. Biomarker results showed decreased circulatory myeloid derived suppressor cell levels and increased CD8+ infiltration into tumors across multiple tumor types, indicating enhanced proinflammatory microenvironment. Data from Part B demonstrate that combination treatment of SRK-181 and pembrolizumab is associated with enhanced proinflammatory microenvironment with promising efficacy in anti-PD(L)-1 resistant patients across multiple tumor types with a manageable safety profile. The results support SRK-181 as a potential treatment to overcome immune checkpoint inhibitor associated resistance.

| | Enrolled pts (n) | | | | |
|---|---|---|---|---|---|
| | ccRCC | HNSCC | MEL | UC | NSCLC |
| | (N = 30) | (N = 9) | (N = 11) | (N = 11) | (N = 11) |
| Efficacy | 30 | 6 | 10 | 11 | 11 |
| Evaluable pts¹ | | | | | |
| (n) | | | | | |
| ORR | 20% | 33.3% | 20% | 9.1% | 0% |
| PR (n) | 6 | 2 | 2 | 1 | 0 |
| Tumor | 38.8%-98.2% | 35.9%-48% | 41%-56.3% | 39% | — |
| Reduction | | | | | |
| DoR² (months) | 0.7+~17.9+ | 0.1~1.4+ | 3.8+~7.1+ | 13.2+ | — |
| SD (n) | 11 | 1 | 5 | 4 | 3 |

¹Ongoing pts are pending first post-treatment radiographic evaluation are not efficacy evaluable
²DoR: duration of response

### Example 6: TGFβ1-Selective Antibodies Block Ascorbate-Mediated Release of TGFβ1 Growth Factor

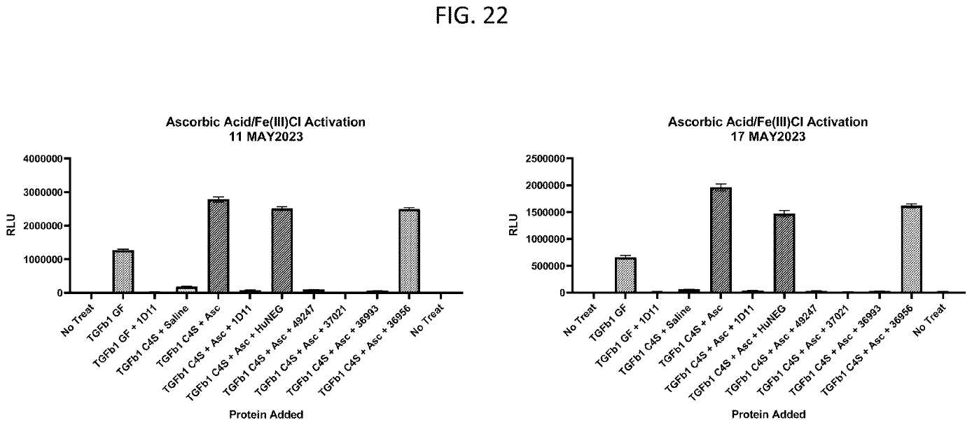

TGFβ1 C4S small latent complex was treated with 3 mM ascorbic acid, 20 pM Fe(III) chloride (Fe(III)CI), and 200 μM EDTA in Chelex-100 Saline at 37° C. for 2 h before being contacted with CAGA12 reporter cells. Data from the CAGA reporter assay showed that ascorbic acid/Fe(III)CI-mediated TGFβ1 growth factor release from SLC was blocked by TGFβ1-selective antibodies, including SRK-181 (also referred to as Ab 36993) and Ab46 (also referred to as Ab 37021) (FIG. 22).

### Example 7: TGFβ1-Selective Antibodies Block Kallikrein Cleavage of Latency-Associated Peptide (LAP)

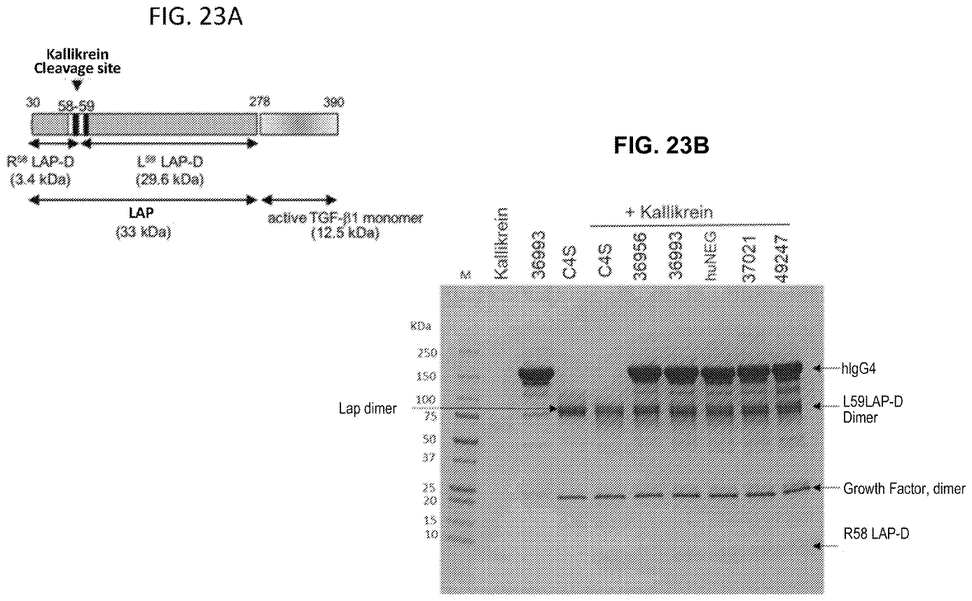

TGFβ1 C4S small latent complex was incubated with a TGFβ1-selective antibody at a weight ratio of 1:5 at room temperature overnight and then was digested with kallikerein at a weight ratio of 20:1 (C4S:kallikerein) at 37° C. for 2.5 h. The digested products were analyzed on a non-reduced gel as shown in FIGS. 23A and 23 B. The results showed that none of the tested TGFβ1-selective antibodies, including SRK-181 (also referred to as Ab 36993) and Ab46 (also referred to as Ab 37021), could block kallikerein cleavage of LAP.

### EQUIVALENTS

The various features and embodiments of the present disclosure, referred to in individual sections above apply, as appropriate, to other sections, mutatis mutandis. Consequently, features specified in one section may be combined with features specified in other sections, as appropriate.

Those skilled in the art will recognize, or be able to ascertain using no more than routine experimentation, many equivalents to the specific embodiments of the disclosure described herein. Such equivalents are intended to be encompassed by the following claims.

1. A TGFβ1 inhibitor for use in the treatment of cancer in a patient who has received prior therapy for the cancer, wherein the treatment comprises administering the TGFβ1 inhibitor in an amount sufficient to inhibit the growth of a solid tumor or reduce the volume of a solid tumor, wherein the cancer is renal cell carcinoma (RCC), head and neck squamous cell carcinoma (HNSCC), ovarian cancer, melanoma, testicular cancer, colorectal cancer, pancreatic cancer, squamous cell skin carcinoma, triple-negative breast cancer (TNBC), or liver cancer.

2. The TGFβ1 inhibitor for use according to claim 1, wherein the prior cancer therapy comprises checkpoint inhibitor therapy, chemotherapy, and/or radiation therapy.

3. The TGFβ1 inhibitor for use according to claim 1 or 2, wherein the prior cancer therapy comprises anti-PD-(L)1 (e.g., pembrolizumab, nivolumab, cemiplimab, atezolizumab, dostarlimab, durvalumab, avelumab), anti-CTLA4 (e.g., ipilimumab, tremelimumab), tyrosine kinase inhibitors (e.g., sunitinib, cabozantinib, imatinib, gefitinib, sorafenib, erlotinib, lapatinib, canertinib, semaxinib, vatalanib, leflunomide, etc.), phosphoinositide 3-kinase (PI3K) inhibitors, paclitaxel, carboplatin, topotecan, doxil, gemcitabine, altretamine, bevacizumab, letrozole, carboplatin/taxol, leuprorelin, carboplatin/docetaxel, and/or bevacizumab.

4. The TGFβ1 inhibitor for use according to any one of claims 1-3, wherein the patient has received multiple lines of prior cancer therapies aimed to treat the cancer.

5. The TGFβ1 inhibitor for use according to any one of claims 1-4, wherein the cancer was resistant or unresponsive to the prior cancer therapies.

6. The TGFβ1 inhibitor for use according to any one of claims 1-5, wherein the disease progressed during the prior therapies.

7. The TGFβ1 inhibitor for use according to any one of claims 1-6, wherein the patient experienced adverse events in response to the prior cancer therapies, leading to the discontinuation of the therapy or therapies.

8. The TGFβ1 inhibitor for use according to any one of claims 1-7, wherein the cancer is from RCC, HNSCC, ovarian carcinoma, TNBC, pancreatic adenocarcinoma, colorectal carcinoma, or squamous cell skin carcinoma.

9. The TGFβ1 inhibitor for use according to any one of claims 1-8, wherein the patient has a metastasis (i.e., a primary cancer has metastasized) at screening (prior to initiating the treatment of the TGFβ1 inhibitor), wherein optionally, the cancer has metastasized to multiple sites.

10. The TGFβ1 inhibitor for use according to any one of claims 1-9, wherein a therapeutically effective amount of the TGFβ1 inhibitor is administered to the patient, wherein optionally, the therapeutically effective amount is:
(i) an amount that achieves a stable disease (SD), e.g., no disease progression for 16 weeks or longer (e.g., 6 months, 7 months, 8 months, 9 months, 10 months, or longer) upon/during the treatment;
(ii) an amount that achieves a partial response (PR), e.g., 30% or greater tumor reduction. In some embodiments, tumor reduction is measured by percent change in sum of diameters (SOD) in target lesions from baseline, wherein further optionally, the therapeutically effective amount achieves 50% or greater reduction in SOD from baseline.

11. The TGFβ1 inhibitor for use according to any one of claims 1-10, wherein the TGFβ1 inhibitor comprises an agent aimed to reduce and capable of reducing the TGFβ1 signaling pathway, wherein optionally the agent is selected from:
(i) inhibitors of TGFβ1 activators, such as integrins that bind the RGD motif within the LAP domain of latent TGFβ1;
(ii) inhibitors of TGFβ1 activation, such as antibodies that bind latent TGFβ1 thereby inhibiting the release of the growth factor from the latent complex;
(iii) inhibitors of the mature (soluble) TGFβ1 ligand, such as neutralizing antibodies, ligand traps that incorporate ligand-binding modules of the TGFβ receptor(s), and nucleic acid-based inhibitors, e.g., siRNA and antisense oligonucleotides; and,
(iv) TGFβ receptor antagonists, such as Alk5 inhibitors.

12. The TGFβ1 inhibitor for use according to any one of claims 1-11, wherein the TGFβ1 inhibitor preferentially inhibits TGFβ1 over TGFβ2 and/or TGFβ3.

13. The TGFβ1 inhibitor for use according to any one of claims 1-11, wherein the TGFβ1 inhibitor preferentially inhibits TGFβ1 and TGFβ2 over TGFβ3.

14. The TGFβ1 inhibitor for use according to any one of claims 1-11, wherein TGFβ1 inhibitor is a TGFβ1-selective inhibitor, wherein optionally the TGFβ1-selective inhibitor is SRK-181.

15. The TGFβ1 inhibitor for use according to any one of claims 1-11, wherein the TGFβ1 inhibitor is or comprises: SRK-181 (by Scholar Rock), RG6440 (SOF10) (by Roche/Chugai), ABBV-151 (livmoniplimab) (by AbbVie), NIS793 (XOMA-089) (by Novartis), PLN-10195 (by Pliant), ES014 (by Elpiscience), Cotsiranib (STP705) (by Sirnaomics), Bintrafusp alpha (M7824), Dalutrafusp alpha (AGEN14423), BMS-986416 (AVID200), MK-2225 (by MERCK), PM8001 (by Biotheus), Vactosertib (by Medpacto), BCA101 (by Bicara), TU2218 (NCE401) (by TiUM), ATB-301 (by Autotelic Bio/Clinigen), AdAPT-001 (AIM-001) (by EpicentRx), CART-PSMA-TGF-bRDN (by Tmunity Therapeutics), HCW9218 (by HCW Biologics), SH3051 (by Sanhome), TST005 (by Transcenta), GS19 (GT90008) (by Kintor (Gensun)), BJ-005 (by BJ Bioscience), QLS31901 (by Qilu Pharmaceutical), TQB2858 (by Chia Tal-tianqing), Y101D (by YZY Biopharma), Charis 1000 (C1K) (by Ensol Biosciences), and/or Fresolimumab (GC1008).

16. The TGFβ1 inhibitor for use according to any one of claims 1-15, wherein the TGFβ1 inhibitor is selected from the antibodies and antigen-binding fragments thereof disclosed in the following publications, as well as those that compete or cross-compete for antigen binding (e.g., sharing overlapping epitopes) with such antibodies: WO 2020/104460, WO 2020/014473, WO 2019/163927, WO 2021/039945, WO 2015/015003, WO 2018/013939, WO 2021/142427, WO 2016/161410, WO 2019/075090, and WO 2020/160291.

17. A TGFβ1-selective inhibitor for use in the treatment of ovarian cancer in a patient, wherein the TGFβ1-selective inhibitor is an antibody that binds a latent TGFβ1 complex thereby inhibiting its activation, wherein the treatment comprises administration of the TGFβ1-selective inhibitor so as to achieve stable disease (SD) for 6 months or longer, wherein optionally the TGFβ1-selective inhibitor is SRK-181 dosed as monotherapy at 240-2400 mg Q3W or Q2W, and wherein the patient received prior cancer therapy, wherein further optionally the prior cancer therapy comprises Paclitaxel/Carboplatin, Topotecan, Doxil, Gemcitabine, Altretamine, Bevacizumab/Gemcitabine/Carboplatin, Letrozole, Carboplatin/Taxol, Leuprorelin, Carboplatin/Docetaxel, Carboplatin/Docetaxel/Bevacizumab, or any combinations thereof.

18. A TGFβ1-selective inhibitor for use in the treatment of RCC, HNSCC, melanoma, or squamous cell skin carcinoma, in a patient whose disease progressed in prior anti-PD-(L)1 therapy, wherein the TGFβ1-selective inhibitor is an antibody that binds a latent TGFβ1 complex thereby inhibiting its activation, wherein the treatment comprises administration of the TGFβ1-selective inhibitor in combination with an anti-PD-(L)1 therapy, wherein optionally the TGFβ1-selective inhibitor is SRK-181 dosed at 240-2400 mg Q3W or Q2W (e.g., 1500 mg Q3W or 1000 mg Q2W).

19. The TGFβ1 inhibitor for use according to any of one of claims 1-18, wherein the cancer comprises a solid tumor, wherein the solid tumor is a CD8+ T cell-infiltrated tumor.

20. The TGFβ1 inhibitor for use according to any one fo claim 1-19, wherein a tumor microenvironment (TME) of the CD8+ T cell-infiltrated tumor has a high Treg/CD8+ T cell ratio prior to the treatment.

21. The TGFβ1 inhibitor for use according to any one fo claim 1-20, wherein CD8+ T cells in the TME express low levels of a proinflammatory cytokine and/or a cytotoxic enzyme prior to the treatment.

22. The TGFβ1 inhibitor for use according to claim 21, wherein the proinflammatory cytokine comprises IFNγ and the cytotoxic enzyme comprises perforin and granzyme A/B.

23. The TGFβ1 inhibitor for use according to any one of claims 1-22, wherein the patient has elevated levels of circulating MDSCs, preferably gMDSCs, prior to treatment.

24. The TGFβ1 inhibitor for use according to any of one of claims 1-23, wherein the patient has an elevated number of platelets in the cancer prior to the treatment.

25. The TGFβ1 inhibitor for use according to any of one of claims 1-24, wherein TGFβ1 is over-expressed in the cancer prior to the treatment.

26. The TGFβ1 inhibitor for use according to any of one of claims 1-25, wherein an TGFβ1 activator is detected at an increased level in the cancer prior to the treatment.

27. The TGFβ1 inhibitor for use according to claim 26, wherein the TGFβ1 activator comprises an integrin capable of binding an RGD motif and comprising alpha-v, alpha-5, alpha-11, beta-6, and/or beta-8.

28. The TGFβ1 inhibitor for use according to claim 26, where the TGFβ1 activator comprises kallikrein, chemotrypsin, trypsin, elastase, plasmin, thrombin, zinc metalloprotease (MMP), and/or ADAM protease.

29. The TGFβ1 inhibitor for use according to any of one of claims 1-28, wherein an increase in a ROS marker is present and/or is expected to be present in the patient prior to and/or during the treatment, wherein the ROS marker comprises isoprostanes (IsoPs), malondialdehyde (MDA), nitrotyrosine, S-glutathionylation, myeloperoxidase (MPO), oxidized low-density lipoprotein (OxLDL), and/or antioxidant enzymes.

30. The TGFβ1 inhibitor for use according to any of one of claims 1-29, wherein an increased deposition of extracellular matrix (ECM) is detected in the cancer prior to the treatment, wherein the increased deposition of ECM is indicated by the level of an ECM marker comprising collagen, fibronectin, and/or fibrillin and/or by the level of a cancer-associated fibroblast (CAF) marker comprising actin alpha, platelet-derived growth factor receptor alpha (PDGFRα/CD140a), platelet-derived growth factor receptor beta (PDGFRβ/CD140b), fibroblast specific protein 1 (FSP-1/S100A4), fibroblast activation protein (FAP), and/or nicotinamide N-methyltransferase (NNMT).

31. The TGFβ1 inhibitor for use according to any of one of claims 1-30, wherein a decreased level of an epithelial marker and/or an increased level of a mesenchymal marker is detected in the cancer prior to the treatment, wherein the epithelial marker comprises E-cadherin, α-catenin, γ-catenin, and/or cytokeratin and the mesenchymal marker comprises fibronectin, vimentin, and/or N-cadherin.

32. The TGFβ1 inhibitor for use according to any of one of claims 1-31, wherein an immunosuppression marker is detected in the cancer prior to the treatment wherein the immunosuppression marker comprises a Treg marker and/or LRRC33, and the Treg marker comprises CD4, FOXP3, and/or CD25.

33. The TGFβ1 inhibitor for use according to any of one of claims 1-32, wherein the cancer is SMAD4-deficient.

34. The TGFβ1 inhibitor for use according to any of one of claims 1-33, wherein the cancer is methylthioadenosine phosphorylase (MTAP)-deficient.

35. The TGFβ1 inhibitor for use according to claim 34, wherein
(A) the cancer has a deletion in the 9p21 locus, and optionally further contains an additional deletion of a tumor suppressor gene;
(B) the cancer has reduced expression of MTAP; and/or
(C) a mutant MTAP with decreased activity is produced in the cancer.

36. The TGFβ1 inhibitor for use according to any of one of claims 1-35, wherein a low level of interferon gamma (IFNγ) is detected in the cancer prior to the treatment.

37. The TGFβ1 inhibitor for use according to any of one of claims 1-36, wherein the treatment comprises
(i) measuring a biomarker in the patient,
(ii) administering the TGFβ1 inhibitor in an amount sufficient to inhibit the growth of a solid tumor or reduce the volume of a solid tumor to the patient,
(iii) measuring the biomarker after administration of the TGFβ1 inhibitor; and
(iv) continuing to administer the treatment if a change in the biomarker is detected.

38. The TGFβ1 inhibitor for use according to claim 37, wherein the biomarker comprises
(A) a TGFβ1 transcript and/or a TGFβ1 protein,
(B) an integrin capable of binding an RGD motif and comprising alpha-v, alpha-5, alpha-11, beta-6, and/or beta-8,
(C) a protease capable of activating TGFβ1,
(D) a ROS marker,
(E) an ECM marker,
(F) a CAF marker,
(G) an epithelial marker,
(H) a mesenchymal marker,
(I) an immunosuppression marker,
(J) a SMAD4 gene, a SMAD4 transcript, and/or a SMAD4 protein,
(K) an MTAP gene, an MTAP transcript, and/or an MTAP protein,
(L) IFNγ,
(M) circulating MDSCs,
(N) platelets,
(O) a heightened Treg/CD8+ T cell ratio in the CD8+ T cell-infiltrated solid tumor, and/or
(P) CD8+ T cells expressing low levels of a proinflammatory cytokine and/or a cytotoxic enzyme in the CD8+ T cell-infiltrated solid tumor.

39. The TGFβ1 inhibitor for use according to claim 37, wherein the step (iii) of the treatment comprises detecting in the cancer a deletion in the 9p21 locus and optimally further detecting an additional deletion in a tumor suppressor gene.

40. A TGFβ1 inhibitor for use in the treatment of cancer in a patient, wherein the treatment comprises
(i) administering the TGFβ1 inhibitor in an amount sufficient to inhibit the growth of a solid tumor or reduce the volume of a solid tumor; and
(ii) administering a genotoxic therapy before and/or concurrently with administration of the TGFβ1 inhibitor, or administering a genotoxic therapy concurrently with and/or after administration of the TGFβ1 inhibitor.

41. The TGFβ1 inhibitor for use according to claim 40, wherein the genotoxic therapy administration starts at least one hour after the initiation of the TGFβ1 inhibitor administration.

42. The TGFβ1 inhibitor for use according to claim 40, wherein the genotoxic therapy administration starts at least one day after the initiation of the TGFβ1 inhibitor administration.

43. The TGFβ1 inhibitor for use according to claim 40, wherein the genotoxic therapy administration starts at least one week after the initiation of the TGFβ1 inhibitor administration.

44. The TGFβ1 inhibitor for use according to any one of claims 40-43, wherein the treatment further comprises continuing to administer the TGFβ1 inhibitor after the genotoxic therapy ends.

45. The TGFβ1 inhibitor for use according to any one of claims 40-44, wherein the genotoxic therapy is a chemotherapy and/or a radiation therapy.

46. The TGFβ1 inhibitor for use according to any one of claims 40-45, wherein the chemotherapy comprises administration of 5-FU, paclitaxel, cisplatin, and/or bleomycin to the patient.

47. The TGFβ1 inhibitor for use according to any one of claims 40-46, wherein the patient further receives a checkpoint inhibitor therapy.

48. The TGFβ1 inhibitor for use according to any one of claims 40-47, wherein the patient has received a checkpoint inhibitor therapy, chemotherapy, and/or radiation therapy of the cancer prior to receiving the TGFβ1 inhibitor.

49. The TGFβ1 inhibitor for use according to any one of claims 40-48, wherein the TGFβ1 inhibitor is a TGFβ1-selective inhibitor, and optionally the TGFβ1-selective inhibitor is SRK-181.

50. The TGFβ1 inhibitor for use according to any one of claims 40-49, wherein the cancer is RCC, HNSCC, ovarian cancer, melanoma, testicular cancer, colorectal cancer, pancreatic cancer, squamous cell skin carcinoma, TNBC, or liver cancer.

51. The TGFβ1 inhibitor for use according to any one of claims 1-50, wherein the cancer is ccRCC.

52. A TGFβ1 inhibitor for use in the treatment of a cancer in a subject, wherein the treatment comprises administration of a TGFβ1 inhibitor to the subject in an amount effective to treat the cancer, wherein CD8+ T cells have infiltrated a tumor microenvironment (TME) in the cancer, and wherein the cancer is resistant or refractory to a cancer therapy, wherein optionally the cancer therapy comprises a checkpoint inhibitor and/or a genotoxic agent therapy, wherein further optionally, the genotoxic agent therapy is radiation therapy or chemotherapy.

53. The TGFβ1 inhibitor for use according to claim 52, wherein the TME has a high Treg/CD8+ T cell ratio prior to the treatment.

54. The TGFβ1 inhibitor for use according to claim 52 or 53, wherein CD8+ T cells in the TME express low levels of a proinflammatory cytokine and/or a cytotoxic enzyme prior to the treatment.

55. The TGFβ1 inhibitor for use according to claim 54, wherein the proinflammatory cytokine comprises IFNγ and the cytotoxic enzyme comprises perforin and granzyme A/B.

56. The TGFβ1 inhibitor for use according to any one of claims 52-55, wherein the patient has elevated levels of circulating MDSCs, preferably gMDSCs, prior to treatment.

57. The TGFβ1 inhibitor for use according to any of one of claims 52-56, wherein the patient has an elevated number of platelets in the cancer prior to the treatment.

58. The TGFβ1 inhibitor for use according to any one of claims 52-57, wherein the subject is further treated with a cancer therapy, selected from checkpoint inhibitors and genotoxic agent therapies.

59. The TGFβ1 inhibitor for use according to any one of claim 52-58, wherein the cancer is undergoing or has undergone epithelial-to-mesenchymal transition (EMT).

60. The TGFβ1 inhibitor for use according to any one of claims 52-59, wherein the cancer comprises a greater number of Treg cells than non-cancerous/normal tissue.

61. The TGFβ1 inhibitor for use according to any one of claims 52-60, wherein a biological sample collected from the subject shows elevated levels of TGFB1 and/or TGFβ1 transcripts, an integrin capable of binding an RGD motif, a protease capable of activating TGFβ1, a ROS marker, a mesenchymal marker; a CAF marker; an immunosuppression marker, Treg cells, platelets, circulating MDSCs, and/or Treg/CD8+ T cell ratio.

62. The TGFβ1 inhibitor for use according to any one of claims 52-60, wherein a biological sample collected from the subject shows decreased levels of epithelial markers, SMAD4 transcripts and/or proteins, methylthioadenosine phosphorylase (MTAP) transcripts and/or proteins, a proinflammatory cytokine, and/or a cytotoxic enzyme.

63. The TGFβ1 inhibitor for use according to any one of claims 52-62, wherein the cancer is selected from renal cell carcinoma (RCC), head and neck squamous cell carcinoma (HNSCC), ovarian cancer, melanoma, testicular cancer, colorectal cancer, pancreatic cancer, squamous cell skin carcinoma, triple-negative breast cancer (TNBC), and liver cancer.

64. The TGFβ1 inhibitor for use according to any one of claims 52-63, wherein the cancer is ccRCC.

65. The TGFβ1 inhibitor for use according to any one of claims 52-64, wherein the TGFβ1 inhibitor is SRK-181.
